(19) United States
(12) Patent Application Publication
(10) Pub. No.: US 2021/0338725 A1
(43) Pub. Date: Nov. 4, 2021

(54) TREATMENT METHODS

(71) Applicant: Genocea Biosciences, Inc., Cambridge, MA (US)

(72) Inventors: Lisa K. McNeil, Watertown, MA (US); Victoria L. DeVault, Watertown, MA (US); James R. Perry, Lexington, MA (US); Hubert Lam, Quincy, MA (US); Adrienne V. Li, Cambridge, MA (US)

(21) Appl. No.: 17/221,569

(22) Filed: Apr. 2, 2021

Related U.S. Application Data
(60) Provisional application No. 63/111,434, filed on Nov. 9, 2020.
(60) Provisional application No. 63/023,708, filed on May 12, 2020.
(60) Provisional application No. 63/004,388, filed on Apr. 2, 2020.

(51) Int. Cl. A61K 35/17; A61K 9/00; A61P 35/00; C12N 5/0784; C12N 5/0783

(52) U.S. Cl. CPC A61K 35/17; A61K 9/0019; A61P 35/00; C12N 2502/1121; C12N 5/0636; C12N 2502/1114; C12N 5/0639

(57) ABSTRACT

Methods and compositions for identifying tumor antigens of human lymphocytes, and for treating subjects having cancer, are provided herein.

### CROSS REFERENCE TO RELATED APPLICATIONS

This application claims the benefit of U.S. Provisional Application No. 63/004,388, filed Apr. 2, 2020, U.S. Provisional Application No. 63/023,708, filed May 12, 2020, and U.S. Provisional Application No. 63/111,434, filed Nov. 9, 2020, the contents of each of which are hereby incorporated by reference herein in their entirety.

### SEQUENCE LISTING

The instant application contains a Sequence Listing which has been submitted electronically in ASCII format and is hereby incorporated by reference in its entirety. Said ASCII copy, created on Jun. 11, 2021, is named 2007781-0307_SL.txt and is 2,106 KB bytes in size.

### BACKGROUND

Checkpoint inhibitor and adoptive tumor infiltrating lymphocytes (TIL) transfer therapies have achieved clinical responses in cancer patients demonstrating the importance of tumor antigen T cell targeting to destroy tumors. Yet, only a fraction of patients benefit from treatment. Checkpoint inhibitors are prone to off-target toxicity and are most successful against tumors with high mutational burden. TIL therapies are limited to indications where bulk tumors are accessible and have high TIL content. They are also derived from non-specific expansion of T cells from a single tumor which limits tumor antigen targeting and makes treatment more prone to metastatic tumor escape. Other cell therapy approaches, in which T cells are engineered to express a chimeric antigen receptor (CAR-T) or antigen-specific T cell receptors (TCR) have also shown limited success but are generally restricted to a single antigen specificity and therefore also prone to tumor escape. There remains a need for additional therapeutic approaches to treat tumors.

### SUMMARY

One aspect of the disclosure includes a method of obtaining a plurality of lymphocytes selectively stimulated by one or more stimulatory antigens. In some embodiments, the stimulatory antigens are specific (personal) to a subject. In some embodiments, the stimulatory antigens are shared by a cohort of subjects. In some embodiments, the stimulatory antigens comprise both specific (personal) and shared stimulatory antigens. In some embodiments, the method comprises obtaining a sample of PBMCs (e.g., by apheresis) from a subject having a tumor or a cancer; isolating from the sample of PBMCs a population of lymphocytes (e.g., T cells) and a population of monocytes; differentiating the monocytes into dendritic cells; selecting one or more stimulatory antigens by: a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject; b) contacting the bacterial cells or beads with antigen presenting cells (APCs) (e.g., a first batch of the dendritic cells) from the subject, wherein the APCs internalize the bacterial cells or beads; c) contacting the APCs with a first batch of the population of lymphocytes from the subject, under conditions suitable for activation of lymphocytes by a polypeptide presented by one or more APCs; d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer, and/or (iii) one or more tumor antigens that inhibit and/or suppress level of expression and/or secretion of one or more immune mediators associated with at least one deleterious and/or non-beneficial response to cancer; co-culturing a second batch of the dendritic cells with (i) a second batch of the population of lymphocytes (e.g., T cells), and (ii) a plurality of overlapping peptides or nucleic acids, e.g., one or more plasmids, wherein the overlapping peptides comprise, or nucleic acids encode, all or part of the amino acid sequence of the one or more stimulatory antigens; and selecting or enriching from the culture a plurality of lymphocytes (e.g., T cells) selectively stimulated by the one or more stimulatory antigens.

In some embodiments, the method of obtaining a plurality of selectively stimulated lymphocytes comprises first selecting shared stimulatory antigens from a cohort of subjects having a cancer or tumor by obtaining a sample of PBMCs (e.g., by apheresis) from each subject; isolating from each sample of PBMCs a population of lymphocytes (e.g., T cells) and a population of monocytes; differentiating the monocytes into dendritic cells; selecting one or more stimulatory antigens by: a) obtaining, providing, or generating a library for each subject comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject; b) contacting the bacterial cells or beads with antigen presenting cells (APCs) (e.g., a first batch of the dendritic cells) from the subject, wherein the APCs internalize the bacterial cells or beads; c) contacting the APCs with a first batch of the population of lymphocytes from the subject, under conditions suitable for activation of lymphocytes by a polypeptide presented by one or more APCs; d) determining for each subject whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying for each subject one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; f) identifying tumor antigens shared across subjects of the cohort; and g) selecting as one or more shared stimulatory antigens, from among the shared tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer, and/or (iii) one or more tumor antigens that inhibit and/or suppress level of expression and/or secretion of one or more immune mediators associated with at least one deleterious and/or non-beneficial response to cancer. The method further comprises obtaining a sample of PBMCs (e.g., by apheresis) from a subject having a tumor or a cancer of the same class as the cohort used to identify and select shared stimulatory antigens; isolating from the subject's sample of PBMCs a population of lymphocytes (e.g., T cells) and a population of monocytes; differentiating the monocytes into dendritic cells; co-culturing the dendritic cells with (i) a population of lymphocytes (e.g., T cells), and (ii) a plurality of overlapping peptides or nucleic acids, e.g., one or more plasmids, wherein the overlapping peptides comprise, or nucleic acids encode, all or part of the amino acid sequence of the one or more shared stimulatory antigens; and selecting or enriching from the culture a plurality of lymphocytes (e.g., T cells) selectively stimulated by the one or more shared stimulatory antigens.

In some embodiments, the population of monocytes comprises CD14⁺ monocytes. In some embodiments, the population of lymphocytes (e.g., T cells) comprises CD4⁺ and/or CD8⁺ T cells.

In some embodiments, the population of lymphocytes (e.g., T cells) is selectively stimulated by one or more subject-specific (personal) antigens. In some embodiments, the population of lymphocytes (e.g., T cells) is selectively stimulated by one or more shared stimulatory antigens. In some embodiments, the population of lymphocytes (e.g., T cells) is selectively stimulated by one or more subject-specific (personal) antigens and one or more shared stimulatory antigens.

In some embodiments, the method further comprises expanding and/or restimulating the plurality of lymphocytes (e.g., T cells). In some embodiments, restimulating comprises contacting the plurality of lymphocytes (e.g., T cells) with the plurality of overlapping peptides or nucleic acids, e.g., one or more plasmids. In some embodiments, restimulating comprises contacting the plurality of lymphocytes (e.g., T cells) with monocyte-derived dendritic cells (MDDCs) pulsed with the plurality of overlapping peptides or nucleic acids, e.g., one or more plasmids. In some embodiments, the method further comprises selecting or enriching, from the plurality of lymphocytes (e.g., T cells), lymphocytes (e.g., T cells) selectively stimulated by the one or more stimulatory antigens.

In some embodiments, the method further comprises non-selectively expanding and/or stimulating the selected or enriched lymphocytes (e.g., T cells). In some embodiments, non-selectively expanding and/or stimulating comprises contacting the plurality of lymphocytes (e.g., T cells) with an anti-CD3 antibody, an anti-CD28 antibody, and/or an anti-CD2 antibody.

In some embodiments, the method further comprises formulating the plurality of lymphocytes (e.g., T cells) as a composition. In some embodiments, the composition comprises a diluent, human serum albumin, and/or DMSO. In some embodiments, the composition is cryo-preserved.

In another aspect, the disclosure features a method of manufacturing a pharmaceutical composition. In some embodiments, the method comprises obtaining a sample of PBMCs (e.g., by apheresis) from a subject having a tumor or a cancer; isolating from the sample of PBMCs a population of lymphocytes (e.g., T cells) and a population of monocytes; differentiating the monocytes into dendritic cells; selecting one or more stimulatory antigens by: a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject; b) contacting the bacterial cells or beads with antigen presenting cells (APCs) (e.g., a first batch of the dendritic cells), wherein the APCs internalize the bacterial cells or beads; c) contacting the APCs with a first batch of the population of lymphocytes, under conditions suitable for activation of lymphocytes by a polypeptide presented by one or more APCs; d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer, and/or (iii) one or more tumor antigens that inhibit and/or suppress level of expression and/or secretion of one or more immune mediators associated with at least one deleterious and/or non-beneficial response to cancer; co-culturing a second batch of the dendritic cells with (i) a second batch of the population of lymphocytes (e.g., T cells), and (ii) a plurality of overlapping peptides or nucleic acids, e.g., one or more plasmids, wherein the overlapping peptides comprise, or nucleic acids encode, all or part of the amino acid sequence of one or more stimulatory antigens; selecting or enriching from the culture a plurality of lymphocytes (e.g., T cells) selectively stimulated by the one or more stimulatory antigens; expanding the plurality of selectively stimulated lymphocytes (e.g., T cells); and formulating the expanded selectively stimulated lymphocytes (e.g., T cells) as a pharmaceutical composition.

In other embodiments, the method of manufacturing a pharmaceutical composition comprises first selecting shared stimulatory antigens from a cohort of subjects having a cancer or tumor by obtaining a sample of PBMCs (e.g., by apheresis) from each subject; isolating from each sample of PBMCs a population of lymphocytes (e.g., T cells) and a population of monocytes; differentiating the monocytes into dendritic cells; selecting one or more stimulatory antigens by: a) obtaining, providing, or generating a library for each subject comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject; b) contacting the bacterial cells or beads with antigen presenting cells (APCs) (e.g., a first batch of the dendritic cells) from the subject, wherein the APCs internalize the bacterial cells or beads; c) contacting the APCs with a first batch of the population of lymphocytes from the subject, under conditions suitable for activation of lymphocytes by a polypeptide presented by one or more APCs; d) determining for each subject whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying for each subject one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; f) identifying tumor antigens shared across subjects of the cohort; and g) selecting as one or more shared stimulatory antigens, from among the shared tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer, and/or (iii) one or more tumor antigens that inhibit and/or suppress level of expression and/or secretion of one or more immune mediators associated with at least one deleterious and/or non-beneficial response to cancer. The method further comprises obtaining a sample of PBMCs (e.g., by apheresis) from a subject having a tumor or a cancer of the same class as the cohort used to identify and select shared stimulatory antigens; isolating from the subject's sample of PBMCs a population of lymphocytes (e.g., T cells) and a population of monocytes; differentiating the monocytes into dendritic cells; co-culturing the dendritic cells with (i) a population of lymphocytes (e.g., T cells), and (ii) a plurality of overlapping peptides or nucleic acids, e.g., one or more plasmids, wherein the overlapping peptides comprise, or nucleic acids encode, all or part of the amino acid sequence of the one or more shared stimulatory antigens; selecting or enriching from the culture a plurality of lymphocytes (e.g., T cells) selectively stimulated by the one or more shared stimulatory antigens; expanding the plurality of selectively stimulated lymphocytes (e.g., T cells); and formulating the expanded selectively stimulated lymphocytes (e.g., T cells) as a pharmaceutical composition.

In some embodiments, the population of lymphocytes (e.g., T cells) is selectively stimulated by one or more subject-specific (personal) antigens. In some embodiments, the population of lymphocytes (e.g., T cells) is selectively stimulated by one or more shared stimulatory antigens. In some embodiments, the population of lymphocytes (e.g., T cells) is selectively stimulated by one or more subject-specific (personal) antigens and one or more shared stimulatory antigens.

In another aspect, the disclosure features a method of conferring an immune response to a subject having a tumor or a cancer. In some embodiments, the method comprises obtaining a sample of PBMCs (e.g., by apheresis) from the subject; isolating from the sample of PBMCs a population of lymphocytes (e.g., T cells) and a population of monocytes; differentiating the monocytes into dendritic cells; selecting one or more stimulatory antigens by: a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject; b) contacting the bacterial cells or beads with antigen presenting cells (APCs) (e.g., a first batch of the dendritic cells), wherein the APCs internalize the bacterial cells or beads; c) contacting the APCs with a first batch of the population of lymphocytes, under conditions suitable for activation of lymphocytes by a polypeptide presented by one or more APCs; d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer; co-culturing a second batch of the dendritic cells with (i) a second batch of the population of lymphocytes (e.g., T cells), and (ii) a plurality of overlapping peptides or nucleic acids, e.g., one or more plasmids, wherein the overlapping peptides comprise or nucleic acids encode, all or part of the amino acid sequence of one or more stimulatory antigens; selecting or enriching from the culture a plurality of lymphocytes (e.g., T cells) selectively stimulated by the one or more stimulatory antigens; expanding the plurality of selectively stimulated lymphocytes (e.g., T cells); and administering the expanded selectively stimulated lymphocytes (e.g., T cells) to the subject, thereby conferring an immune response to the tumor or cancer.

In other embodiments, the method of conferring an immune response comprises first selecting shared stimulatory antigens from a cohort of subjects having a cancer or tumor by obtaining a sample of PBMCs (e.g., by apheresis) from each subject; isolating from each sample of PBMCs a population of lymphocytes (e.g., T cells) and a population of monocytes; differentiating the monocytes into dendritic cells; selecting one or more stimulatory antigens by: a) obtaining, providing, or generating a library for each subject comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject; b) contacting the bacterial cells or beads with antigen presenting cells (APCs) (e.g., a first batch of the dendritic cells) from the subject, wherein the APCs internalize the bacterial cells or beads; c) contacting the APCs with a first batch of the population of lymphocytes from the subject, under conditions suitable for activation of lymphocytes by a polypeptide presented by one or more APCs; d) determining for each subject whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying for each subject one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; f) identifying tumor antigens shared across subjects of the cohort; and g) selecting as one or more shared stimulatory antigens, from among the shared tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer, and/or (iii) one or more tumor antigens that inhibit and/or suppress level of expression and/or secretion of one or more immune mediators associated with at least one deleterious and/or non-beneficial response to cancer. The method further comprises obtaining a sample of PBMCs (e.g., by apheresis) from a subject having a tumor or a cancer of the same class as the cohort used to identify and select shared stimulatory antigens; isolating from the subject's sample of PBMCs a population of lymphocytes (e.g., T cells) and a population of monocytes; differentiating the monocytes into dendritic cells; co-culturing the dendritic cells with (i) a population of lymphocytes (e.g., T cells), and (ii) a plurality of overlapping peptides or nucleic acids, e.g., one or more plasmids, wherein the overlapping peptides comprise, or nucleic acids encode, all or part of the amino acid sequence of the one or more shared stimulatory antigens; selecting or enriching from the culture a plurality of lymphocytes (e.g., T cells) selectively stimulated by the one or more shared stimulatory antigens; expanding the plurality of selectively stimulated lymphocytes (e.g., T cells); and administering the expanded selectively stimulated lymphocytes (e.g., T cells) to the subject, thereby conferring an immune response to the tumor or cancer.

In some embodiments, the population of lymphocytes (e.g., T cells) is selectively stimulated by one or more subject-specific (personal) antigens. In some embodiments, the population of lymphocytes (e.g., T cells) is selectively stimulated by one or more shared stimulatory antigens. In some embodiments, the population of lymphocytes (e.g., T cells) is selectively stimulated by one or more subject-specific (personal) antigens and one or more shared stimulatory antigens.

In another aspect, the disclosure features a method of treating a subject having a tumor or a cancer. In some embodiments, the method comprises obtaining a sample of PBMCs (e.g., by apheresis) from the subject; isolating from the sample of PBMCs a population of lymphocytes (e.g., T cells) and a population of monocytes; differentiating the monocytes into dendritic cells; selecting one or more stimulatory antigens by: a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject; b) contacting the bacterial cells or beads with antigen presenting cells (APCs) (e.g., a first batch of the dendritic cells), wherein the APCs internalize the bacterial cells or beads; c) contacting the APCs with a first batch of the population of lymphocytes, under conditions suitable for activation of lymphocytes by a polypeptide presented by one or more APCs; d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer; co-culturing a second batch of the dendritic cells with (i) a second batch of the population of lymphocytes (e.g., T cells), and (ii) a plurality of overlapping peptides or nucleic acids, e.g., one or more plasmids, wherein the overlapping peptides comprise, or nucleic acids encode, all or part of the amino acid sequence of one or more stimulatory antigens; selecting or enriching from the culture a plurality of lymphocytes (e.g., T cells) selectively stimulated by the one or more stimulatory antigens; expanding the plurality of selectively stimulated lymphocytes (e.g., T cells); and administering the expanded selectively stimulated lymphocytes (e.g., T cells) to the subject, thereby treating the tumor or cancer.

In other embodiments, the method of treating a subject comprises first selecting shared stimulatory antigens from a cohort of subjects having a cancer or tumor by obtaining a sample of PBMCs (e.g., by apheresis) from each subject; isolating from each sample of PBMCs a population of lymphocytes (e.g., T cells) and a population of monocytes; differentiating the monocytes into dendritic cells; selecting one or more stimulatory antigens by: a) obtaining, providing, or generating a library for each subject comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject; b) contacting the bacterial cells or beads with antigen presenting cells (APCs) (e.g., a first batch of the dendritic cells) from the subject, wherein the APCs internalize the bacterial cells or beads; c) contacting the APCs with a first batch of the population of lymphocytes from the subject, under conditions suitable for activation of lymphocytes by a polypeptide presented by one or more APCs; d) determining for each subject whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying for each subject one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; f) identifying tumor antigens shared across subjects of the cohort; and g) selecting as one or more shared stimulatory antigens, from among the shared tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer, and/or (iii) one or more tumor antigens that inhibit and/or suppress level of expression and/or secretion of one or more immune mediators associated with at least one deleterious and/or non-beneficial response to cancer. The method further comprises obtaining a sample of PBMCs (e.g., by apheresis) from a subject having a tumor or a cancer of the same class as the cohort used to identify and select shared stimulatory antigens; isolating from the subject's sample of PBMCs a population of lymphocytes (e.g., T cells) and a population of monocytes; differentiating the monocytes into dendritic cells; co-culturing the dendritic cells with (i) a population of lymphocytes (e.g., T cells), and (ii) a plurality of overlapping peptides or nucleic acids, e.g., one or more plasmids, wherein the overlapping peptides comprise, or nucleic acids encode, all or part of the amino acid sequence of the one or more shared stimulatory antigens; selecting or enriching from the culture a plurality of lymphocytes (e.g., T cells) selectively stimulated by the one or more shared stimulatory antigens; expanding the plurality of selectively stimulated lymphocytes (e.g., T cells); and administering the expanded selectively stimulated lymphocytes (e.g., T cells) to the subject, thereby treating the tumor or cancer.

In some embodiments, the population of lymphocytes (e.g., T cells) is selectively stimulated by one or more subject-specific (personal) antigens. In some embodiments, the population of lymphocytes (e.g., T cells) is selectively stimulated by one or more shared stimulatory antigens. In some embodiments, the population of lymphocytes (e.g., T cells) is selectively stimulated by one or more subject-specific (personal) antigens and one or more shared stimulatory antigens.

In some embodiments, the library comprises bacterial cells or beads comprising at least 1, 3, 5, 10, 15, 20, 25, 30, 50, 100, 150, 250, 500, 750, 1000 or more different heterologous polypeptides, or portions thereof.

In some embodiments, determining whether the one or more lymphocytes are activated by, or not responsive to, one or more polypeptides comprises measuring a level of one or more immune mediators. In some embodiments, the one or more immune mediators are selected from the group consisting of cytokines, soluble mediators, and cell surface markers expressed by the lymphocytes.

In some embodiments, the one or more immune mediators are cytokines. In some embodiments, the one or more cytokines are selected from the group consisting of TRAIL, IFN-gamma, IL-12p70, IL-2, TNF-alpha, MIP1-alpha, MIP1-beta, CXCL9, CXCL10, MCP1, RANTES, IL-1 beta, IL-4, IL-6, IL-8, IL-9, IL-10, IL-13, IL-15, CXCL11, IL-3, IL-5, IL-17, IL-18, IL-21, IL-22, IL-23A, IL-24, IL-27, IL-31, IL-32, TGF-beta, CSF, GM-CSF, TRANCE (also known as RANK L), MIP3-alpha, and fractalkine.

In some embodiments, the one or more immune mediators are soluble mediators. In some embodiments, the one or more soluble mediators are selected from the group consisting of granzyme A, granzyme B, granzyme K, sFas, sFasL, perforin, and granulysin.

In some embodiments, the one or more immune mediators are cell surface markers. In some embodiments, the one or more cell surface markers are selected from the group consisting of CD107a, CD107b, CD25, CD69, CD45RA, CD45RO, CD137 (4-1BB), CD44, CD62L, CD27, CCR7, CD154 (CD40L), KLRG-1, CD71, HLA-DR, CD122 (IL-2RB), CD28, IL7Ra (CD127), CD38, CD26, CD134 (OX-40), CTLA-4 (CD152), LAG-3, TIM-3 (CD366), CD39, PD1 (CD279), FoxP3, TIGIT, CD160, BTLA, 2B4 (CD244), CCR2, CCR5, CX3CR1, NKG2D, CD39, KLRD1, LGALS1 (encoding Galectin-1), and KLRG1.

In some embodiments, lymphocyte activation is determined by assessing a level of one or more expressed or secreted immune mediators that is at least 20%, 40%, 60%, 80%, 100%, 120%, 140%, 160%, 180%, or 200% higher or lower than a control level. In some embodiments, lymphocyte activation is determined by assessing a level of one or more expressed or secreted immune mediators that is at least one, two, or three standard deviations greater or lower than the mean of a control level. In some embodiments, lymphocyte activation is determined by assessing a level of one or more expressed or secreted immune mediators that is at least 1, 2, 3, 4 or 5 median absolute deviations (MADs) greater or lower than a median response level to a control. In some embodiments, lymphocyte activation is determined by molecular profiling of gene expression, e.g., real-time PCR, of immune mediators.

In some embodiments, lymphocyte non-responsiveness is determined by assessing a level of one or more expressed or secreted immune mediators that is within 5%, 10%, 15%, or 20% of a control level. In some embodiments, lymphocyte non-responsiveness is determined by assessing a level of one or more expressed or secreted immune mediators that is less than one or two standard deviation higher or lower than the mean of a control level. In some embodiments, lymphocyte non-responsiveness is determined by assessing a level of one or more expressed or secreted immune mediators that is less than one or two median absolute deviation (MAD) higher or lower than a median response level to a control. In some embodiments, lymphocyte non-responsiveness is determined by molecular profiling of gene expression, e.g., real-time PCR, of immune mediators.

In some embodiments, the one or more stimulatory antigens comprise (i) a tumor antigen described herein (e.g., comprising an amino acid sequence described herein), (ii) a polypeptide having an amino acid sequence at least 90%, 91%, 92%, 93%, 94%, 95%, 96%, 97%, 98%, 99% identical to the amino acid sequence of a tumor antigen described herein, (iii) a viral gene or portion thereof, and/or (iv) a polypeptide comprising the amino acid sequence of a tumor antigen described herein having at least one mutation, deletion, insertion, and/or translocation.

In some embodiments, the method further comprises producing the plurality of overlapping peptides or nucleic acids, e.g., one or more plasmids, wherein the overlapping peptides comprise, or nucleic acids encode, all or part of the amino acid sequence of one or more stimulatory antigens.

In some embodiments, the method further comprises administering the composition to the subject. In some embodiments, the composition is administered to the subject by intravenous infusion. In some embodiments, the subject suffers from refractory disease. In some embodiments, the subject suffers from advanced refractory disease. In some embodiments, the subject suffers from a solid tumor. In some embodiments, the subject suffers from melanoma, malignant melanoma, Merkel cell carcinoma (MCC), cutaneous squamous cell carcinoma (CSCC), non-small cell lung cancer (NSCLC), small cell lung cancer (SCLC), large cell lung cancer (LCLC), tracheobronchial cancer, pleomorphic carcinoma, squamous cell lung carcinoma (SqCLC), squamous cell carcinoma of the head and neck (SCCHN), nasopharyngeal carcinoma (NPC), urothelial carcinoma (bladder, ureter, urethra, or renal pelvis), renal cell carcinoma (RCC), or anal squamous cell carcinoma (ASCC). In some embodiments, the subject suffers from breast cancer, endometrial cancer, cervical cancer, ovarian cancer, pancreatic cancer, colorectal cancer, prostate cancer, bone cancer, chondrosarcoma, osteosarcoma, or thyroid cancer. In some embodiments, the method further comprises administering to the subject a cancer therapy or combination of cancer therapies, e.g., a therapeutic cancer vaccine, a chemotherapeutic agent, an immune stimulator, or an immune checkpoint therapy.

In another aspect, the disclosure features a method of manufacturing a pharmaceutical composition. In some embodiments, the method comprises obtaining a sample of PBMCs (e.g., by apheresis) from a subject having a tumor or a cancer; isolating from the sample of PBMCs a population of lymphocytes (e.g., T cells) and a population of CD14+ monocytes; separating the sample of lymphocytes into a first batch of lymphocytes and a second batch of lymphocytes; separating the sample of monocytes into a first batch of monocytes and a second batch of monocytes; cryopreserving the first and second batches of monocytes and the first and second batches of lymphocytes and storing each cryopreserved batch for a specified period of time; thawing the first batch of lymphocytes and/or the first batch of monocytes; differentiating the first batch of monocytes into a first batch of dendritic cells; selecting one or more stimulatory antigens by: a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject; b) contacting the bacterial cells or beads with the first batch of dendritic cells, wherein the dendritic cells internalize the bacterial cells or beads; c) contacting the first batch of dendritic cells with the first batch of lymphocytes, under conditions suitable for activation of lymphocytes by a polypeptide presented by one or more of the dendritic cells; d) determining whether one or more lymphocytes are activated by, or not responsive to, one or more polypeptides presented by one or more of the dendritic cells, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and f) selecting one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer; synthesizing a plurality of overlapping peptides, wherein the overlapping peptides comprise all or part of the amino acid sequence of one or more stimulatory antigens; thawing the second batch of lymphocytes and the second batch of monocytes; differentiating the second batch of monocytes into a second batch of dendritic cells; co-culturing the second batch of dendritic cells with (i) the second batch of lymphocytes (e.g., T cells), and (ii) the plurality of overlapping peptides, thereby selectively stimulating the second batch of lymphocytes; selecting or enriching from the co-culture a plurality of lymphocytes (e.g., T cells) selectively stimulated by the one or more stimulatory antigens and selectively expanding the lymphocytes in the presence of one or more cytokines; restimulating the selectively expanded selectively stimulated lymphocytes with the plurality of overlapping peptides and sorting the lymphocytes (e.g., T cells) that express CD137+, CD154+, or CD137+ and CD154+ cell surface markers; further expanding the plurality of selectively stimulated lymphocytes (e.g., T cells) by culturing the sorted lymphocytes in the presence of one or more cytokines and anti-CD3, anti-CD28, and/or anti-CD2 antibodies; formulating the further expanded selectively stimulated lymphocytes (e.g., T cells) as a pharmaceutical composition; and cryopreserving the pharmaceutical composition.

In another aspect, the disclosure features a method of manufacturing a pharmaceutical composition. In some embodiments, the method comprises obtaining a sample of PBMCs (e.g., by apheresis) from a subject having a tumor or a cancer; isolating from the sample of PBMCs a population of lymphocytes (e.g., T cells) and a population of CD14+ monocytes; separating the sample of lymphocytes into a first batch of lymphocytes and a second batch of lymphocytes; separating the sample of monocytes into a first batch of monocytes and a second batch of monocytes; differentiating the first batch of monocytes into a first batch of dendritic cells; selecting one or more stimulatory antigens by: a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject; b) contacting the bacterial cells or beads with the first batch of dendritic cells, wherein the dendritic cells internalize the bacterial cells or beads; c) contacting the first batch of dendritic cells with the first batch of lymphocytes, under conditions suitable for activation of lymphocytes by a polypeptide presented by one or more of the dendritic cells; d) determining whether one or more lymphocytes are activated by, or not responsive to, one or more polypeptides presented by one or more of the dendritic cells, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and f) selecting one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer; synthesizing a plurality of overlapping peptides, wherein the overlapping peptides comprise all or part of the amino acid sequence of one or more stimulatory antigens; differentiating the second batch of monocytes into a second batch of dendritic cells; co-culturing the second batch of dendritic cells with (i) the second batch of lymphocytes (e.g., T cells), and (ii) the plurality of overlapping peptides, thereby selectively stimulating the second batch of lymphocytes; selecting or enriching from the co-culture a plurality of lymphocytes (e.g., T cells) selectively stimulated by the one or more stimulatory antigens and selectively expanding the lymphocytes in the presence of one or more cytokines; restimulating the selectively expanded, selectively stimulated lymphocytes with the plurality of overlapping peptides and sorting the lymphocytes (e.g., T cells) that express CD137+, CD154+, or CD137+ and CD154+ cell surface markers; further expanding the plurality of selectively stimulated lymphocytes (e.g., T cells) by culturing the sorted lymphocytes in the presence of one or more cytokines and anti-CD3, anti-CD28, and/or anti-CD2 antibodies; and formulating the further expanded, selectively stimulated lymphocytes (e.g., T cells) as a pharmaceutical composition.

In some embodiments, the one or more cytokines comprise one or more cytokines selected from the group consisting of: IL-2, IL-7, IL-15, and IL-21. In some embodiments, the sorted lymphocytes are cultured in the presence of a superantigen or a mitogen, e.g., phorbol 12-myristate 13-acetate (PMA), ionomycin, phytohemagglutinin (PHA), or Concanavalin A (ConA).

In another aspect, the disclosure includes pharmaceutical compositions comprising a plurality of selectively stimulated lymphocyte, e.g., T cells, or expanded and selectively stimulated lymphocytes, e.g, T cells, obtained by any method of the disclosure.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 is a graph showing normalized CD8⁺ T cell response levels, measured by production of either IFN-gamma (panel A) or TNF-alpha (panel B), against different mutated tumor proteins.

FIG. 2 is a Venn diagram showing limited overlap between CD8⁺ T cell stimulatory and inhibitory antigens identified using methods of the disclosure and epitope prediction algorithms.

FIG. 3 is a diagram of exemplary methods used to rank stimulatory and inhibitory antigens of the disclosure. Three screens were run measuring IFN-gamma and TNF-alpha (panel A) and a ranked list was generated based on the three screens (panels B and C).

FIG. 4 is a diagram of exemplary methods to show effects of adoptive cell therapy on tumor progression, using T cells primed in vivo by vaccination with stimulatory or inhibitory antigens identified using methods of the disclosure.

FIG. 5 shows an exemplary overall workflow for ATLAS-based, patient-specific antigen identification and selection, followed by antigen-specific T cell stimulation and expansion, yielding an exemplary autologous adoptive cell therapy.

FIG. 6 shows workflow for an exemplary autologous adoptive cell therapy manufacturing process of the disclosure.

FIG. 7 shows workflow for the antigen identification and selection portion of an exemplary autologous adoptive cell therapy manufacturing process of the disclosure.

FIG. 8 is a graph showing antigen-specific T cell expansion when sorted CD4⁺ and/or CD8⁺ T cell are cultured with model peptide antigens (of viral origin) and monocyte-derived dendritic cells (MDDCs), total peripheral blood mononuclear cells (PBMCs), or T cell depleted-PBMCs. Fold expansion of viable cells after 10-day expansion is shown.

FIG. 9 shows phenotypic characteristics of immature and mature MDDCs. CD14⁺ monocytes were differentiated and matured using methods of the disclosure. Panel A is an image of differentiated immature dendritic cells (DCs) at Day 6, showing numerous dendrites, a hallmark of DCs. Panel B is an image of mature DCs at Day 8 (harvest). The cells have rounded up and are easily accessible for harvest. Panel C shows histograms for typical MDDC maturation markers at Day 8. Grey shaded: Isotype controls, Black lines: specific antibodies.

FIG. 10 is a graph showing T cell expansion of sorted CD4⁺, CD8⁺, or CD4⁺ and CD8⁺ T cells cultured in the presence of MDDCs pulsed with model peptide antigens (of viral origin). Co-culture of CD4⁺ and CD8⁺ T cells did not impact individual expansion of each T cell subset. Fold expansion of viable cells after 10-day expansion is depicted.

FIG. 11 is a set of graphs showing purity of sorted CD4⁺ and CD8⁺ T cells cultured in the presence of MDDCs pulsed with model peptide antigens (of viral origin). T cells were re-stimulated with model peptide antigens on Day 10 for 15 hours, then labeled with CD137/CD154 antibodies and magnetic beads. Labeled cells were separated on magnetic selection columns. Cells were stained for CD3 (T cell marker) and CD137/CD154 (activation markers) to visualize cell populations.

FIG. 12 is a graph comparing T cell expansion using two different non-specific stimuli. T cells were cultured for 12 days with low-dose IL-2 and either anti-CD3 and CD28 monoclonal antibodies (T Cell TransACT, Miltenyi Biotec) or anti-CD3, CD28, and CD2 monoclonal antibodies (ImmunoCult, StemCell Technologies). Fold expansion and percent viability of the two cultures were determined.

FIG. 13 shows T cell expansion and percentage of central memory phenotype under different culture conditions. T cells were cultured with anti-CD3 and CD28 antibody and various combinations of cytokines for 10 days. Panel A lists cytokine combinations for different media. Panel B shows fold expansion of the cultures. Panel C shows the memory phenotype of CD4⁺ T cells characterized by CD45RA and CCR7 expression. A central memory phenotype (CM) is identified as CCR7 high and CD45RA low; effector memory (EM) phenotype is CD45RA low and CCR7 low; effector memory re-expressing CD45RA (TEMRA) is CD45RA high and CCR7 negative. Panel D shows the memory phenotype of CD8⁺ T cells characterized as in Panel C.

FIG. 14, Panel A shows the number of T cells present prior to T cell expansion (pre-expansion cells) and post-expansion from co-culture with peptide antigens and monocyte-derived dendritic cells, followed by sorting for activation markers and non-specific expansion using anti-CD3/CD28 microbeads (exemplary process yielding exemplary autologous adoptive cell therapy product GEN-011). Data are combined responses from eight development runs using T cells (and stimulatory tumor antigens) from three cancer patients or T cells (and model antigens of viral origin) from five healthy donors. Panel B shows the proportion of T cells and CD4⁺ or CD8⁺ T cell subsets post-expansion. Panel C shows memory phenotypes (central or effector) of the antigen-specific T cells. A central memory phenotype (CM) is identified as CCR7 high and CD45RA low; effector memory (EM) phenotype is identified as CD45RA low and CCR7 low; effector memory re-expressing CD45RA (TEMRA) phenotype is identified as CD45RA high and CCR7 negative.

FIG. 15 shows the number of cytokine-secreting cells in response to antigens prior to T cell expansion (pre-expansion cells) and post-expansion from co-culture with peptide antigens and monocyte-derived dendritic cells followed by sorting for activation markers and non-specific expansion using anti-CD3/CD28 microbeads (exemplary process yielding exemplary autologous adoptive cell therapy product GEN-011). Data are combined responses from five development runs using T cells (and stimulatory tumor antigens) from three cancer patients or T cells (and model antigens of viral origin) from two healthy donors. Data are shown as the cytokine spot forming units (SFU) per million T cells as detected by a dual-analyte FluoroSpot assay measuring IFN-gamma and TNF-alpha.

FIG. 16 shows results for the exemplary autologous adoptive cell therapy compositions compared to publicly reported Tumor Infiltrating Lymphocyte (TIL) data. Panel A left bar shows the proportion of T cells with activation marker (CD137/CD154) up-regulation after specific antigen re-stimulation of exemplary cell therapy compositions. The right bar shows the reported up-regulation of activation markers in TIL compositions. Panel B left bar shows IFN-gamma levels in supernatants after specific antigen re-stimulation of exemplary cell therapy compositions, as detected by ELISA assays. These are compared to TIL compositions stimulated non-specifically with anti-CD3 and -28 antibodies (right bar). Squares represent cancer patient cell therapy compositions re-stimulated with stimulatory tumor antigens, circles represent healthy donor cell therapy compositions re-stimulated with model antigens of viral origin.

FIG. 17 shows phenotypic characterization by flow cytometry of an exemplary autologous adoptive cell therapy composition. Panel A shows the proportion of CD4⁺ and CD8⁺ T cell subsets present after stimulation and expansion. Panel B shows phenotypes of the non-T cell subset (<0.6% of total). Panel C shows memory phenotypes (central or effector) of the antigen-specific T cells. A central memory phenotype (CM) is identified as CCR7 high and CD45RA low; effector memory (EM) phenotype is identified as CD45RA low and CCR7 low; effector memory re-expressing CD45RA (TEMRA) phenotype is identified CD45RA high and CCR7 negative.

FIG. 18 shows antigen specificity, as measured by a T cell activation assay, of an exemplary autologous adoptive cell therapy composition. Cells were stimulated for 15 hours with DMSO as negative control, or with a pool of overlapping peptides corresponding to a patient's ATLAS-identified and selected antigens.

FIG. 19 shows that an exemplary autologous adoptive cell therapy composition induces dose-dependent killing of the exemplary patient's stimulated autologous APC, compared to unstimulated autologous APC.

FIG. 20 shows robust polyfunctional cytokine responses in an exemplary autologous adoptive cell therapy composition.

FIG. 21 shows results of TCRβ sequencing of pre-expansion PBMCs compared to exemplary autologous adoptive cell therapy compositions. Panel A shows representative tree maps of the top 25 TCRs for each sample; each spot represents a unique V-J-CDR3 and size represents frequency. (NB. Spots are not comparable across squares.) The upper box shows representative results for pre-expansion PBMCs from one cancer patient. The lower box represents the final TCRβ results in the patient's corresponding GEN-011 drug product, DEV4. Panel B shows the frequency of the top 25 CDR3s in GEN-011 drug products indicated on the x axis (dark gray bars), plotted relative to their pre-expansion frequencies (light gray bars).

FIG. 22 shows T cell characteristics of exemplary autologous adoptive cell therapy compositions at the antigen-specific expansion phase, during which T cells were cultured in the presence of either fresh MDDCs (Fresh) or cryopreserved MDDCs (Frozen) pulsed with model overlapping peptide antigens. Panel A shows glucose levels (solid lines, indicating mmol/L of supernatant in the Fresh and Frozen arms of the study) and lactate levels (dashed lines, indicating mmol/L of supernatant in the Fresh and Frozen arms) expressed by antigen-specifically expanded T cells over days 12 to 17 in culture. Panel B shows the fold-expansion of T cells in response to specific antigens in the Fresh and Frozen arms, on days 17 and 18 in culture. Panel C shows the percent viability of antigen-specifically expanded T cells in the Fresh and Frozen arms, from days 12 to 18 in culture.

FIG. 23 shows T cell characteristics of exemplary autologous adoptive cell therapy compositions at the rapid, non-specific expansion phase, following the antigen-specific expansion phase shown in FIG. 22. Panel A shows glucose levels (solid lines; indicating mmol/L of supernatant in the Fresh and Frozen arms of the study) and lactate levels (dashed lines, indicating mmol/L of supernatant in the Fresh and Frozen arms) expressed by non-specifically expanded T cells over days 24 to 29 in culture. Panel B shows the percent viability of non-specifically expanded T cells in the Fresh and Frozen arms on days 18 and 29 in culture.

### DEFINITIONS

Activate or Stimulate: As used herein, a peptide presented by an antigen presenting cell (APC) “activates”, or equivalently, “stimulates” a lymphocyte if lymphocyte activity is detectably modulated after exposure to the peptide presented by the APC under conditions that permit antigen-specific recognition to occur. Any indicator of lymphocyte activity can be evaluated to determine whether a lymphocyte is activated or stimulated, e.g., T cell proliferation, phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be systemic or local. In some embodiments, administration may be enteral or parenteral. In some embodiments, administration may be by injection (e.g., intramuscular, intravenous, or subcutaneous injection). In some embodiments, injection may involve bolus injection, drip, perfusion, or infusion (e.g., intravenous infusion). In some embodiments administration may be topical. Those skilled in the art will be aware of appropriate administration routes for use with particular therapies described herein, for example from among those listed on www.fda.gov, which include auricular (otic), buccal, conjunctival, cutaneous, dental, endocervical, endosinusial, endotracheal, enteral, epidural, extra-amniotic, extracorporeal, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracorporus cavernosum, intradermal, intranodal, intradiscal, intraductal, intraduodenal, intradural, intraepidermal, intraesophageal, intragastic, intragingival, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, oropharyngeal, parenteral, percutaneous, periarticular, peridural, perineural, periodontal, rectal, respiratory (e.g., inhalation), retrobulbar, soft tissue, subarachnoid, subconjunctival, subcutaneous, sublingual, submucosal, topical, transdermal, transmucosal, transplacental, transtracheal, ureteral, urethral, or vaginal. In some embodiments, administration may involve electro-osmosis, hemodialysis, infiltration, iontophoresis, irrigation, and/or occlusive dressing. In some embodiments, administration may involve dosing that is intermittent (e.g., a plurality of doses separated in time) and/or periodic (e.g., individual doses separated by a common period of time) dosing. In some embodiments, administration may involve continuous dosing.

Adoptive cell therapy: As used herein, “adoptive cell therapy” or “ACT” involves the transfer of cells (e.g., immune cells) into a subject (e.g., a subject having cancer). In some embodiments, ACT is a treatment approach that involves the use of lymphocytes with anti-tumor activity, the in vitro expansion of these cells to suitable numbers, and their infusion into a subject having cancer.

Antigen: The term “antigen”, as used herein, refers to a molecule (e.g., a polypeptide) that elicits a specific immune response. Antigen-specific immunological responses, also known as adaptive immune responses, are mediated by lymphocytes (e.g., T cells, B cells, NK cells) that express antigen receptors (e.g., T cell receptors, B cell receptors). In certain embodiments, an antigen is a T cell antigen, and elicits a cellular immune response. In certain embodiments, an antigen is a B cell antigen, and elicits a humoral (i.e., antibody) response. In certain embodiments, an antigen is both a T cell antigen and a B cell antigen. As used herein, the term “antigen” encompasses both a full-length polypeptide as well as a portion or immunogenic fragment of the polypeptide, and a peptide epitope within the polypeptides (e.g., a peptide epitope bound by a Major Histocompatibility Complex/Human Leukocyte Antigen (MHC/HLA) molecule (e.g., MHC/HLA class I, or MHC/HLA class II)).

Antigen presenting cell: An “antigen presenting cell” or “APC” refers to a cell that presents peptides on MHC/HLA class I and/or MHC/HLA class II molecules for recognition by T cells. APC include both professional APC (e.g., dendritic cells, macrophages, B cells), which have the ability to stimulate naïve lymphocytes, and non-professional APC (e.g., fibroblasts, epithelial cells, endothelial cells, glial cells). In certain embodiments, APC are able to internalize (e.g., endocytose) members of a library (e.g., cells of a library of bacterial cells) that express heterologous polypeptides as candidate antigens.

Autolysin polypeptide: An “autolysin polypeptide” is a polypeptide that facilitates or mediates autolysis of a cell (e.g., a bacterial cell) that has been internalized by a eukaryotic cell. In some embodiments, an autolysin polypeptide is a bacterial autolysin polypeptide. Autolysin polypeptides include, and are not limited to, polypeptides whose sequences are disclosed in GenBank® under Acc. Nos. NP_388823.1, NP_266427.1, and P0AGC3.1.

Cancer: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T cell lymphoma, Hodgkin lymphoma, mycosis fungoides, Sezary syndrome, AIDS-related lymphoma, follicular lymphoma, diffuse large B-cell lymphoma), melanoma, Merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

Cytolysin polypeptide: A “cytolysin polypeptide” is a polypeptide that has the ability to form pores in a membrane of a eukaryotic cell. A cytolysin polypeptide, when expressed in host cell (e.g., a bacterial cell) that has been internalized by a eukaryotic cell, facilitates release of host cell components (e.g., host cell macromolecules, such as host cell polypeptides) into the cytosol of the internalizing cell. In some embodiments, a cytolysin polypeptide is bacterial cytolysin polypeptide. In some embodiments, a cytolysin polypeptide is a cytoplasmic cytolysin polypeptide. Cytolysin polypeptides include, and are not limited to, polypeptides whose sequences are disclosed in U.S. Pat. No. 6,004,815, and in GenBank® under Acc. Nos. NP_463733.1, NP_979614, NP_834769, YP_084586, YP_895748, YP_694620, YP_012823, NP_346351, YP_597752, BAB41212.2, NP_561079.1, YP_001198769, and NP_359331.1.

Cytoplasmic cytolysin polypeptide: A “cytoplasmic cytolysin polypeptide” is a cytolysin polypeptide that has the ability to form pores in a membrane of a eukaryotic cell, and that is expressed as a cytoplasmic polypeptide in a bacterial cell. A cytoplasmic cytolysin polypeptide is not significantly secreted by a bacterial cell. Cytoplasmic cytolysin polypeptides can be provided by a variety of means. In some embodiments, a cytoplasmic cytolysin polypeptide is provided as a nucleic acid encoding the cytoplasmic cytolysin polypeptide. In some embodiments, a cytoplasmic cytolysin polypeptide is provided attached to a bead. In some embodiments, a cytoplasmic cytolysin polypeptide has a sequence that is altered relative to the sequence of a secreted cytolysin polypeptide (e.g., altered by deletion or alteration of a signal sequence to render it nonfunctional). In some embodiments, a cytoplasmic cytolysin polypeptide is cytoplasmic because it is expressed in a secretion-incompetent cell. In some embodiments, a cytoplasmic cytolysin polypeptide is cytoplasmic because it is expressed in a cell that does not recognize and mediate secretion of a signal sequence linked to the cytolysin polypeptide. In some embodiments, a cytoplasmic cytolysin polypeptide is a bacterial cytolysin polypeptide.

Heterologous: The term “heterologous”, as used herein to refer to genes or polypeptides, refers to a gene or polypeptide that does not naturally occur in the organism in which it is present and/or being expressed, and/or that has been introduced into the organism by the hand of man. In some embodiments, a heterologous polypeptide is a tumor antigen described herein.

Immune mediator: As used herein, the term “immune mediator” refers to any molecule that affects the cells and processes involved in immune responses. Immune mediators include cytokines, chemokines, soluble proteins, and cell surface markers.

Improve, increase, inhibit, stimulate, suppress, or reduce: As used herein, the terms “improve”, “increase”, “inhibit”, “stimulate”, “suppress”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. The effect of a particular agent or treatment may be direct or indirect. In some embodiments, an appropriate reference measurement may be or may comprise a measurement in a comparable system known or expected to respond in a particular way, in presence of the relevant agent or treatment. In some embodiments, a peptide presented by an antigen presenting cell (APC) “stimulates” or is “stimulatory” to a lymphocyte if the lymphocyte is activated to a phenotype associated with beneficial responses, after exposure to the peptide presented by the APC under conditions that permit antigen-specific recognition to occur, as observed by, e.g., T cell proliferation, phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers, relative to a control. In some embodiments, a peptide presented by an antigen presenting cell “suppresses”, “inhibits” or is “inhibitory” to a lymphocyte if the lymphocyte is activated to a phenotype associated with deleterious or non-beneficial responses, after exposure to the peptide presented by the APC under conditions that permit antigen-specific recognition to occur, as observed by, e.g., phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers, relative to a control.

Inhibitory Antigen: An “inhibitory antigen” or “inhibitory tumor antigen” is an antigen that elicits an immune response with the potential to inhibit, suppress, impair and/or reduce immune control of a tumor or cancer in a subject. In some embodiments, an inhibitory antigen promotes tumor growth, enables tumor growth, ameliorates tumor growth, activates tumor growth, accelerates tumor growth, and/or increases and/or enables tumor metastasis. In some embodiments, an inhibitory antigen stimulates one or more lymphocyte responses that are deleterious or non-beneficial to a subject; and/or inhibits and/or suppresses one or more lymphocyte responses that are beneficial to a subject. In some embodiments, an inhibitory antigen is the target of one or more lymphocyte responses that are deleterious or non-beneficial to a subject; and/or inhibits and/or suppresses one or more lymphocyte responses that are beneficial to a subject.

Invasin polypeptide: An “invasin polypeptide” is a polypeptide that facilitates or mediates uptake of a cell (e.g., a bacterial cell) by a eukaryotic cell. Expression of an invasin polypeptide in a noninvasive bacterial cell confers on the cell the ability to enter a eukaryotic cell. In some embodiments, an invasin polypeptide is a bacterial invasin polypeptide. In some embodiments, an invasin polypeptide is a Yersinia invasin polypeptide (e.g., a Yersinia invasin polypeptide comprising a sequence disclosed in GenBank® under Acc. No. YP_070195.1).

Listeriolysin O (LLO): The terms “listeriolysin O” or “LLO” refer to a listeriolysin O polypeptide of Listeria monocytogenes and truncated forms thereof that retain pore-forming ability (e.g., cytoplasmic forms of LLO, including truncated forms lacking a signal sequence). In some embodiments, an LLO is a cytoplasmic LLO. Exemplary LLO sequences are shown in Table 1, below.

Polypeptide: The term “polypeptide”, as used herein, generally has its art-recognized meaning of a polymer of at least three amino acids. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) and immunogenic fragments of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides.

Primary cells: As used herein, “primary cells” refers to cells from an organism that have not been immortalized in vitro. In some embodiments, primary cells are cells taken directly from a subject (e.g., a human). In some embodiments, primary cells are progeny of cells taken from a subject (e.g., cells that have been passaged in vitro). Primary cells include cells that have been stimulated to proliferate in culture.

Response: As used herein, in the context of a subject (a patient or experimental organism), “response”, “responsive”, or “responsiveness” refers to an alteration in a subject's condition that occurs as a result of, or correlates with, treatment. In certain embodiments, a response is a beneficial response. In certain embodiments, a beneficial response can include stabilization of a subject's condition (e.g., prevention or delay of deterioration expected or typically observed to occur absent the treatment), amelioration (e.g., reduction in frequency and/or intensity) of one or more symptoms of the condition, and/or improvement in the prospects for cure of the condition, etc. In certain embodiments, for a subject who has cancer, a beneficial response can include: the subject has a positive clinical response to cancer therapy or a combination of therapies; the subject has a spontaneous response to a cancer; the subject is in partial or complete remission from cancer; the subject has cleared a cancer; the subject has not had a relapse, recurrence or metastasis of a cancer; the subject has a positive cancer prognosis; the subject has not experienced toxic responses or side effects to a cancer therapy or combination of therapies. In certain embodiments, for a subject who had cancer, the beneficial responses occurred in the past, or are ongoing.

In certain embodiments, a response is a deleterious or non-beneficial response. In certain embodiments, a deleterious or non-beneficial response can include deterioration of a subject's condition, lack of amelioration (e.g., no reduction in frequency and/or intensity) of one or more symptoms of the condition, and/or degradation in the prospects for cure of the condition, etc. In certain embodiments, for a subject who has cancer, a deleterious or non-beneficial response can include: the subject has a negative clinical response to cancer therapy or a combination of therapies; the subject is not in remission from cancer; the subject has not cleared a cancer; the subject has had a relapse, recurrence or metastasis of a cancer; the subject has a negative cancer prognosis; the subject has experienced toxic responses or side effects to a cancer therapy or combination of therapies. In certain embodiments, for a subject who had cancer, the deleterious or non-beneficial responses occurred in the past, or are ongoing.

As used herein, in the context of a cell, organ, tissue, or cell component, e.g., a lymphocyte, “response”, “responsive”, or “responsiveness” refers to an alteration in cellular activity that occurs as a result of, or correlates with, administration of or exposure to an agent, e.g. a tumor antigen. In certain embodiments, a beneficial response can include increased expression and/or secretion of immune mediators associated with positive clinical responses or outcomes in a subject. In certain embodiments, a beneficial response can include decreased expression and/or secretion of immune mediators associated with negative clinical response or outcomes in a subject. In certain embodiments, a deleterious or non-beneficial response can include increased expression and/or secretion of immune mediators associated with negative clinical responses or outcomes in a subject. In certain embodiments, a deleterious or non-beneficial response can include decreased expression and/or secretion of immune mediators associated with positive clinical responses or outcomes in a subject. In certain embodiments, a response is a clinical response. In certain embodiments, a response is a cellular response. In certain embodiments, a response is a direct response. In certain embodiments, a response is an indirect response. In certain embodiments, “non-response”, “non-responsive”, or “non-responsiveness” mean minimal response or no detectable response. In certain embodiments, a “minimal response” includes no detectable response. In certain embodiments, presence, extent, and/or nature of response can be measured and/or characterized according to particular criteria. In certain embodiments, such criteria can include clinical criteria and/or objective criteria. In certain embodiments, techniques for assessing response can include, but are not limited to, clinical examination, positron emission tomography, chest X-ray, CT scan, MM, ultrasound, endoscopy, laparoscopy, presence or level of a particular marker in a sample, cytology, and/or histology. Where a response of interest is a response of a tumor to a therapy, ones skilled in the art will be aware of a variety of established techniques for assessing such response, including, for example, for determining tumor burden, tumor size, tumor stage, etc. Methods and guidelines for assessing response to treatment are discussed in Therasse et al., J. Natl. Cancer Inst., 2000, 92(3):205-216; and Seymour et al., Lancet Oncol., 2017, 18:e143-52. The exact response criteria can be selected in any appropriate manner, provided that when comparing groups of tumors, patients or experimental organism, and/or cells, organs, tissues, or cell components, the groups to be compared are assessed based on the same or comparable criteria for determining response rate. One of ordinary skill in the art will be able to select appropriate criteria.

Stimulatory Antigen: A “stimulatory antigen” or “stimulatory tumor antigen” is an antigen that elicits an immune response with the potential to enhance, improve, increase and/or stimulate immune control of a tumor or cancer in a subject. In some embodiments, a stimulatory antigen is the target of an immune response that reduces, kills, shrinks, resorbs, and/or eradicates tumor growth; does not promote, enable, ameliorate, activate, and/or accelerate tumor growth; decreases tumor metastasis, and/or decelerates tumor growth. In some embodiments, a stimulatory antigen inhibits and/or suppresses one or more lymphocyte responses that are deleterious or non-beneficial to a subject; and/or stimulates one or more lymphocyte responses that are beneficial to a subject.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

### DETAILED DESCRIPTION

Neoantigens are emerging as attractive targets for personalized cancer immunotherapy. Unlike tumor-associated antigens (TAAs) that are recognized as self, neoantigens can contain non-synonymous mutations that may be identified as foreign to the immune system and are not subject to central tolerance.

Recent advances in immune checkpoint inhibitor therapies such as ipilimumab, nivolumab, and pembrolizumab for cancer immunotherapy have resulted in dramatic efficacy in subjects suffering from NSCLC, among other indications. Nivolumab and pembroluzimab have been approved by the Food and Drug Administration (FDA) and European Medicines Agency (EMA) for use in patients with advanced NSCLC who have previously been treated with chemotherapy. They have solidified the importance of T cell responses in control of tumors. Neoantigens, potential cancer rejection antigens that are entirely absent from the normal human genome, are postulated to be relevant to tumor control; however, attempts to define them and their role in tumor clearance has been hindered by the paucity of available tools to define them in a biologically relevant and unbiased way (Schumacher and Schreiber, 2015 Science 348:69-74, Gilchuk et al., 2015 Curr Opin Immunol 34:43-51)

Taking non-small cell lung carcinoma (NSCLC) as an example, whole exome sequencing of NSCLC tumors from patients treated with pembrolizumab showed that higher non-synonymous mutation burden in tumors was associated with improved objective response, durable clinical benefit, and progression-free survival (Rizvi et al., (2015) Science 348(6230): 124-8). In this study, the median non-synonymous mutational burden of the discovery cohort was 209 and of the validation cohort was 200. However, simply because a mutation was identified by sequencing, does not mean that the epitope it creates can be recognized by a T cell or serves as a protective antigen for T cell responses (Gilchuk et al., 2015 Curr Opin Immunol 34:43-51), making the use of the word neoantigen somewhat of a misnomer. With 200 or more potential targets of T cells in NSCLC, it is not feasible to test every predicted epitope to determine which of the mutations serve as neoantigens, and which neoantigens are associated with clinical evidence of tumor control. Recently, a study by McGranahan et al., showed that clonal neoantigen burden and overall survival in primary lung adenocarcinomas are related. However, even enriching for clonal neoantigens results in potential antigen targets ranging from 50 to approximately 400 (McGranahan et al., 2016 Science 351:1463-69). Similar findings have been described for melanoma patients who have responded to ipilimumab therapy (Snyder et al., 2015 NEJM; Van Allen et al., 2015 Science) and in patients with mismatch-repair deficient colorectal cancer who were treated with pembrolizumab (Le et al., 2015 NEJM).

Adoptive T cell therapies (ACT) enriched for neoantigen targeting with tumor infiltrating lymphocytes (TILs) have demonstrated clinical responses in metastatic cancer with limited off-target toxicity (Tran, E., Robbins, P. F. & Rosenberg, S. A. ‘Final common pathway’ of human cancer immunotherapy: targeting random somatic mutations. Nat Immunol 18, 255-262, doi:10.1038/ni.3682 (2017); Zacharakis, N. et al. Immune recognition of somatic mutations leading to complete durable regression in metastatic breast cancer. Nat Med 24, 724-730, doi:10.1038/s41591-018-0040-8 (2018)). While adoptive TIL therapy has produced durable tumor regression in some patients, the majority do not benefit. Furthermore, tumor infiltrating lymphocyte (TIL) therapy is limited to large, resectable tumors with high TIL content.

ATLAS is the only existing platform for rapid, high-throughput quantification of pre-existing, antigen-specific CD4⁺ and CD8⁺ T cell responses without the use of algorithms or in silico downselection criteria, and has previously yielded antigens with clinical efficacy when administered as a vaccine (Bernstein, D. I. et al. Therapeutic Vaccine for Genital Herpes Simplex Virus-2 Infection: Findings From a Randomized Trial. J Infect Dis 215, 856-864, doi:10.1093/infdis/jix004 (2017)). In cancer, ATLAS enables comprehensive screening of a tumor mutanome by using a patient's own autologous immune cells, specifically professional and/or non-professional antigen presenting cells (APCs), e.g., monocyte-derived dendritic cells (MDDC), and sorted CD8⁺ and CD4⁺ T cells. By utilizing autologous APCs and T cells, ATLAS is agnostic to HLA type and assesses pre-existing T cell responses to any given mutation (Nogueira, C., Kaufmann, J. K., Lam, H. & Flechtner, J. B. Improving Cancer Immunotherapies through Empirical Neoantigen Selection. Trends Cancer 4, 97-100, doi:10.1016/j.trecan.2017.12.003 (2018)). Patient antigen presenting cells, e.g., MDDC, are pulsed with an ordered array of Escherichia coli expressing patient-specific mutations as short polypeptides, with or without co-expressed listeriolysin O (cLLO) facilitating HLA class I or class II presentation, respectively. CD8⁺ or CD4⁺ T cells are subsequently added, and after an overnight incubation, antigens are differentially characterized as stimulatory or inhibitory by significant up- or down-regulation of T cell cytokine secretion relative to control responses; thus, the ATLAS assay allows for identification and characterization of desired, as well as potentially unwanted, antigen-specific T cell responses.

The systems and methods described herein improve upon ACT by using ATLAS to identify and select neoantigens or other tumor specific antigens that elicit stimulatory T cell responses from peripheral blood of a patient, and specifically stimulating and expanding these T cells for infusion back to the patient. This personalized ACT is able to target a broad array of tumor antigens, including but not limited to neoantigens, limit metastatic tumor escape, balance tumor antigen-specific CD4⁺ and CD8⁺ T cell content, and broaden indication selection.

The present disclosure provides, in part, methods and systems for the rapid identification of tumor antigens (e.g., tumor specific antigens (TSAs, or neoantigens), tumor associated antigens (TAAs), or cancer/testis antigens (CTAs)) that elicit T cell responses and particularly that elicit human T cell responses, as well as polypeptides that are potential tumor antigens. For purposes of this disclosure, “tumor antigens” includes both tumor antigens and potential tumor antigens. As described herein, methods of the present disclosure identified stimulatory tumor antigens that were not identified by known algorithms. Further, methods of the present disclosure identified suppressive and/or inhibitory tumor antigens that are not identifiable by known algorithms. Methods of the present disclosure also identified polypeptides that are potential tumor antigens, i.e., polypeptides that activate T cells of non-cancerous subjects, but not T cells of subjects suffering from cancer. The present disclosure also provides methods of selecting or deselecting tumor antigens and potential tumor antigens, methods of using the selected or deselected tumor antigens and potential tumor antigens, immunogenic compositions comprising or excluding the selected tumor antigens and potential tumor antigens, and methods of manufacturing immunogenic compositions.

### Library Generation

A library is a collection of members (e.g., cells or non-cellular particles, such as virus particles, liposomes, or beads (e.g., beads coated with polypeptides, such as in vitro translated polypeptides, e.g., affinity beads, e.g., antibody coated beads, or NTA-Ni beads bound to polypeptides of interest). According to the present disclosure, members of a library include (e.g., internally express or carry) polypeptides of interest described herein. In some embodiments, members of a library are cells that internally express polypeptides of interest described herein. In some embodiments, members of a library which are particles carry, and/or are bound to, polypeptides of interest. Use of a library in an assay system allows simultaneous evaluation in vitro of cellular responses to multiple candidate antigens. According to the present disclosure, a library is designed to be internalized by human antigen presenting cells so that peptides from library members, including peptides from internally expressed polypeptides of interest, are presented on HLA molecules of the antigen presenting cells for recognition by T cells.

Libraries can be used in assays that detect peptides presented by HLA class I and HLA class II molecules. Polypeptides expressed by the internalized library members are digested in intracellular endocytic compartments (e.g., phagosomes, endosomes, lysosomes) of the human cells and presented on HLA class II molecules, which are recognized by human CD4⁺ T cells. In some embodiments, library members include a cytolysin polypeptide, in addition to a polypeptide of interest. In some embodiments, library members include an invasin polypeptide, in addition to the polypeptide of interest. In some embodiments, library members include an autolysin polypeptide, in addition to the polypeptide of interest. In some embodiments, library members are provided with cells that express a cytolysin polypeptide (i.e., the cytolysin and polypeptide of interest are not expressed in the same cell, and an antigen presenting cell is exposed to members that include the cytolysin and members that include the polypeptide of interest, such that the antigen presenting cell internalizes both, and such that the cytolysin facilitates delivery of polypeptides of interest to the HLA class I pathway of the antigen presenting cell). A cytolysin polypeptide can be constitutively expressed in a cell, or it can be under the control of an inducible expression system (e.g., an inducible promoter). In some embodiments, a cytolysin is expressed under the control of an inducible promoter to minimize cytotoxicity to the cell that expresses the cytolysin.

Once internalized by a human cell, a cytolysin polypeptide perforates intracellular compartments in the human cell, allowing polypeptides expressed by the library members to gain access to the cytosol of the human cell. Polypeptides released into the cytosol are presented on HLA class I molecules, which are recognized by CD8⁺ T cells.

A library can include any type of cell or particle that can be internalized by and deliver a polypeptide of interest (and a cytolysin polypeptide, in applications where a cytolysin polypeptide is desirable) to, antigen presenting cells for use in methods described herein. Although the term “cell” is used throughout the present specification to refer to a library member, it is understood that, in some embodiments, the library member is a non-cellular particle, such as a virus particle, liposome, or bead. In some embodiments, members of the library include polynucleotides that encode the polypeptide of interest (and cytolysin polypeptide), and can be induced to express the polypeptide of interest (and cytolysin polypeptide) prior to, and/or during internalization by antigen presenting cells.

In some embodiments, the cytolysin polypeptide is heterologous to the library cell in which it is expressed, and facilitates delivery of polypeptides expressed by the library cell into the cytosol of a human cell that has internalized the library cell. Cytolysin polypeptides include bacterial cytolysin polypeptides, such as listeriolysin O (LLO), streptolysin O (SLO), and perfringolysin O (PFO). Additional cytolysin polypeptides are described in U.S. Pat. No. 6,004,815. In certain embodiments, library members express LLO. In some embodiments, a cytolysin polypeptide is not significantly secreted by the library cell (e.g., less than 20%, 10%, 5%, or 1% of the cytolysin polypeptide produced by the cell is secreted). For example, the cytolysin polypeptide is a cytoplasmic cytolysin polypeptide, such as a cytoplasmic LLO polypeptide (e.g., a form of LLO which lacks the N-terminal signal sequence, as described in Higgins et al., Mol. Microbial. 31(6):1631-1641, 1999). Exemplary cytolysin polypeptide sequences are shown in Table 1. The listeriolysin O (Δ3-25) sequence shown in the second row of Table 1 has a deletion of residues 3-25, relative to the LLO sequence in shown in the first row of Table 1, and is a cytoplasmic LLO polypeptide. In some embodiments, a cytolysin is expressed constitutively in a library host cell. In other embodiments, a cytolysin is expressed under the control of an inducible promoter. Cytolysin polypeptides can be expressed from the same vector, or from a different vector, as the polypeptide of interest in a library cell.

TABLE 1
Exemplary Cytolysin Polypeptides

| | Polypeptide | |
|---|---|---|
| Polypeptide Name | Accession No. | |
| (species) | GI No. | Polypeptide Sequence |
| listeriolysin O | NP_463733.1 | MKKIMLVFITLILVSLPIAQQTEAKDASAFNKENSISSMAPPASP |
| (Listeria | GI: 16802248 | PASPKTPIEKKHADEIDKYIQGLDYNKNNVLVYHGDAVTNVPPRK |
| monocytogenes) | | GYKDGNEYIVVEKKKKSINQNNADIQVVNAISSLTYPGALVKANS |
| | | ELVENQPDVLPVKRDSLTLSIDLPGMTNQDNKIVVKNATKSNVNN |
| | | AVNTLVERWNEKYAQAYPNVSAKIDYDDEMAYSESQLIAKFGTAF |
| | | KAVNNSLNVNFGAISEGKMQEEVISFKQIYYNVNVNEPTRPSRFF |
| | | GKAVTKEQLQALGVNAENPPAYISSVAYGRQVYLKLSTNSHSTKV |
| | | KAAFDAAVSGKSVSGDVELTNIIKNSSFKAVIYGGSAKDEVQIID |
| | | GNLGDLRDILKKGATFNRETPGVPIAYTTNFLKDNELAVIKNNSE |
| | | YIETTSKAYTDGKINIDHSGGYVAQFNISWDEVNYDPEGNEIVQH |
| | | KNWSENNKSKLAHFTSSIYLPGNARNINVYAKECTGLAWEWWRTV |
| | | IDDRNLPLVKNRNISIWGTTLYPKYSNKVDNPIE (SEQ ID |
| | | NO: 1) |
| listeriolysin O | | MKDASAFNKENSISSMAPPASPPASPKTPIEKKHADEIDKYIQGL |
| (Δ3-25) | | DYNKNNVLVYHGDAVTNVPPRKGYKDGNEYIVVEKKKKSINQNNA |
| | | DIQVVNAISSLTYPGALVKANSELVENQPDVLPVKRDSLTLSIDL |
| | | PGMTNQDNKIVVKNATKSNVNNAVNTLVERWNEKYAQAYPNVSAK |
| | | IDYDDEMAYSESQLIAKEGTAFKAVNNSLNVNFGAISEGKMQEEV |
| | | ISFKQIYYNVNVNEPTRPSRFFGKAVTKEQLQALGVNAENPPAYI |
| | | SSVAYGRQVYLKLSTNSHSTKVKAAFDAAVSGKSVSGDVELTNII |
| | | KNSSFKAVIYGGSAKDEVQIIDGNLGDLRDILKKGATFNRETPGV |
| | | PIAYTTNFLKDNELAVIKNNSEYIETTSKAYIDGKINIDHSGGYV |
| | | AQFNISWDEVNYDPEGNEIVQHKNWSENNKSKLAHFTSSIYLPGN |
| | | ARNINVYAKECTGLAWEWWRTVIDDRNLPLVKNRNISIWGTTLYP |
| | | KYSNKVDNPIE(SEQ ID NO: 2) |
| streptolysin O | BAB41212.2 | MSNKKTFKKYSRVAGLLTAALIIGNLVTANAESNKQNTASTETTT |
| (Streptococcus | GI: 71061060 | TSEQPKPESSELTIEKAGQKMDDMLNSNDMIKLAPKEMPLESAEK |
| pyogenes) | | EEKKSEDKKKSEEDHTEEINDKIYSLNYNELEVLAKNGETIENFV |
| | | PKEGVKKADKFIVIERKKKNINTTPVDISIIDSVTDRTYPAALQL |
| | | ANKGFTENKPDAVVTKRNPQKIHIDLPGMGDKATVEVNDPTYANV |
| | | STAIDNLVNQWHDNYSGGNTLPARTQYTESMVYSKSQIEAALNVN |
| | | SKILDGTLGIDFKSISKGEKKVMIAAYKQIFYTVSANLPNNPADV |
| | | FDKSVTFKDLQRKGVSNEAPPLFVSNVAYGRTVFVKLETSSKSND |
| | | VEAAFSAALKGTDVKTNGKYSDILENSSFTAVVLGGDAAEHNKVV |
| | | TKDFDVIRNVIKDNATFSRKNPAYPISYTSVFLKNNKIAGVNNRT |
| | | EYVETTSTEYTSGKINLSHQGAYVAQYEILWDEINYDDKGKEVIT |
| | | KRRWDNNWYSKTSPFSTVIPLGANSRNIRIMARECTGLAWEWWRK |
| | | VIDERDVKLSKEINVNISGSTLSPYGSITYK (SEQ ID NO: 3) |
| perfringolysin O | NP_561079.1 | MIRFKKTKLIASIAMALCLFSQPVISFSKDITDKNQSIDSGISSL |
| (Clostridium | GI: 18309145 | SYNRNEVLASNGDKIESFVPKEGKKTGNKFIVVERQKRSLTTSPV |
| perfringens) | | DISIIDSVNDRTYPGALQLADKAFVENRPTILMVKRKPININIDL |
| | | PGLKGENSIKVDDPTYGKVSGAIDELVSKWNEKYSSTHTLPARTQ |
| | | YSESMVYSKSQISSALNVNAKVLENSLGVDFNAVANNEKKVMILA |
| | | YKQIFYTVSADLPKNPSDLFDDSVTFNDLKQKGVSNEAPPLMVSN |
| | | VAYGRTIYVKLETTSSSKDVQAAFKALIKNTDIKNSQQYKDIYEN |
| | | SSFTAVVLGGDAQEHNKVVTKDFDEIRKVIKDNATFSTKNPAYPI |
| | | SYTSVFLKDNSVAAVHNKTDYIETTSTEYSKGKINLDHSGAYVAQ |
| | | FEVAWDEVSYDKEGNEVLTHKTWDGNYQDKTAHYSTVIPLEANAR |
| | | NIRIKARECTGLAWEWWRDVISEYDVPLTNNINVSIWGTTLYPGS |
| | | SITYN (SEQ ID NO: 4) |
| Pneumolysin | NP_359331.1 | MANKAVNDFILAMNYDKKKLLTHQGESIENRFIKEGNQLPDEFVV |
| (Streptococcus | GI: 933687 | IERKKRSLSTNTSDISVTATNDSRLYPGALLVVDETLLENNPTLL |
| pneumoniae) | | AVDRAPMTYSIDLPGLASSDSFLQVEDPSNSSVRGAVNDLLAKWH |
| | | QDYGQVNNVPARMQYEKITAHSMEQLKVKFGSDFEKTGNSLDIDF |
| | | NSVHSGEKQIQIVNFKQIYYTVSVDAVKNPGDVFQDTVTVEDLKQ |
| | | RGISAERPLVYISSVAYGRQVYLKLETTSKSDEVEAAFEALIKGV |
| | | KVAPQTEWKQILDNTEVKAVILGGDPSSGARVVTGKVDMVEDLIQ |
| | | EGSRFTADHPGLPISYTTSFLRDNVVATFQNSTDYVETKVTAYRN |
| | | GDLLLDHSGAYVAQYYITWDELSYDHQGKEVLTPKAWDRNGQDLT |
| | | AHFTTSIPLKGNVRNLSVKIRECTGLAWEWWRTVYEKTDLPLVRK |
| | | RTISIWGTTLYPQVEDKVEND (SEQ ID NO: 5) |

In some embodiments, a library member (e.g., a library member which is a bacterial cell) includes an invasin that facilitates uptake by the antigen presenting cell. In some embodiments, a library member includes an autolysin that facilitates autolysis of the library member within the antigen presenting cell. In some embodiments, a library member includes both an invasin and an autolysin. In some embodiments, a library member which is an E. coli cell includes an invasin and/or an autolysin. In various embodiments, library cells that express an invasin and/or autolysin are used in methods that also employ non-professional antigen presenting cells or antigen presenting cells that are from cell lines. Isberg et al. (Cell, 1987, 50:769-778), Sizemore et al. (Science, 1995, 270:299-302) and Courvalin et al. (C.R. Acad. Sci. Paris, 1995, 318:1207-12) describe expression of an invasin to effect endocytosis of bacteria by target cells. Autolysins are described by Cao et al., Infect. Immun. 1998, 66(6): 2984-2986; Margot et al., J. Bacteriol. 1998, 180(3):749-752; Buist et al., Appl. Environ. Microbiol., 1997, 63(7):2722-2728; Yamanaka et al., FEMS Microbiol. Lett., 1997, 150(2): 269-275; Romero et al., FEMS Microbiol. Lett., 1993, 108(1):87-92; Betzner and Keck, Mol. Gen. Genet., 1989, 219(3): 489-491; Lubitz et al., J. Bacteriol., 1984, 159(1):385-387; and Tomasz et al., J. Bacteriol., 1988, 170(12): 5931-5934. In some embodiments, an autolysin has a feature that permits delayed lysis, e.g., the autolysin is temperature-sensitive or time-sensitive (see, e.g., Chang et al., 1995, J. Bact. 177, 3283-3294; Raab et al., 1985, J. Mol. Biol. 19, 95-105; Gerds et al., 1995, Mol. Microbiol. 17, 205-210). Useful cytolysins also include addiction (poison/antidote) autolysins, (see, e.g., Magnuson R, et al., 1996, J. Biol. Chem. 271(31), 18705-18710; Smith A S, et al., 1997, Mol. Microbiol. 26(5), 961-970).

In some embodiments, members of the library include bacterial cells. In certain embodiments, the library includes non-pathogenic, non-virulent bacterial cells. Examples of bacteria for use as library members include E. coli, mycobacteria, Listeria monocytogenes, Shigella flexneri, Bacillus subtilis, or Salmonella.

In some embodiments, members of the library include eukaryotic cells (e.g., yeast cells). In some embodiments, members of the library include viruses (e.g., bacteriophages). In some embodiments, members of the library include liposomes. Methods for preparing liposomes that include a cytolysin and other agents are described in Kyung-Dall et al., U.S. Pat. No. 5,643,599. In some embodiments, members of the library include beads. Methods for preparing libraries comprised of beads are described, e.g., in Lam et al., Nature 354: 82-84, 1991, U.S. Pat. Nos. 5,510,240 and 7,262,269, and references cited therein.

In certain embodiments, a library is constructed by cloning polynucleotides encoding polypeptides of interest, or portions thereof, into vectors that express the polypeptides of interest in cells of the library. The polynucleotides can be synthetically synthesized. The polynucleotides can be cloned by designing primers that amplify the polynucleotides. Primers can be designed using available software, such as Primer3Plus (available the following URL: bioinformatics.nl/cgi-bin/primer3plus/primer3plus.cgi; see Rozen and Skaletsky, In: Krawetz S, Misener S (eds) Bioinformatics Methods and Protocols: Methods in Molecular Biology. Humana Press, Totowa, N.J., pp. 365-386, 2000). Other methods for designing primers are known to those of skill in the art. In some embodiments, primers are constructed so as to produce polypeptides that are truncated, and/or lack hydrophobic regions (e.g., signal sequences or transmembrane regions) to promote efficient expression. The location of predicted signal sequences and predicted signal sequence cleavage sites in a given open reading frame (ORF) sequence can be determined using available software, see, e.g., Dyrløv et al., J. Mol. Biol., 340:783-795, 2004, and the following URL: cbs.dtu.dk/services/SignalP/). For example, if a signal sequence is predicted to occur at the N-terminal 20 amino acids of a given polypeptide sequence, a primer is designed to anneal to a coding sequence downstream of the nucleotides encoding the N-terminal 20 amino acids, such that the amplified sequence encodes a product lacking this signal sequence.

Primers can also be designed to include sequences that facilitate subsequent cloning steps. ORFs can be amplified directly from genomic DNA (e.g., genomic DNA of a tumor cell), or from polynucleotides produced by reverse transcription (RT-PCR) of mRNAs expressed by the tumor cell. RT-PCR of mRNA is useful, e.g., when the genomic sequence of interest contains intronic regions. PCR-amplified ORFs are cloned into an appropriate vector, and size, sequence, and expression of ORFs can be verified prior to use in immunological assays.

In some embodiments, a polynucleotide encoding a polypeptide of interest is linked to a sequence encoding a tag (e.g., an N-terminal or C-terminal epitope tag) or a reporter protein (e.g., a fluorescent protein). Epitope tags and reporter proteins facilitate purification of expressed polypeptides, and can allow one to verify that a given polypeptide is properly expressed in a library host cell, e.g., prior to using the cell in a screen. Useful epitope tags include, for example, a polyhistidine (His) tag, a V5 epitope tag from the P and V protein of paramyxovirus, a hemagglutinin (HA) tag, a myc tag, and others. In some embodiments, a polynucleotide encoding a polypeptide of interest is fused to a sequence encoding a tag which is a known antigenic epitope (e.g., an MHC/HLA class I- and/or MHC/HLA class II-restricted T cell epitope of a model antigen such as an ovalbumin), and which can be used to verify that a polypeptide of interest is expressed and that the polypeptide-tag fusion protein is processed and presented in antigen presentation assays. In some embodiments a tag includes a T cell epitope of a murine T cell (e.g., a murine T cell line). In some embodiments, a polynucleotide encoding a polypeptide of interest is linked to a tag that facilitates purification and a tag that is a known antigenic epitope. Useful reporter proteins include naturally occurring fluorescent proteins and their derivatives, for example, Green Fluorescent Protein (Aequorea Victoria) and Neon Green (Branchiostoma lanceolatum). Panels of synthetically derived fluorescent and chromogenic proteins are also available from commercial sources.

Polynucleotides encoding a polypeptide of interest are cloned into an expression vector for introduction into library host cells. Various vector systems are available to facilitate cloning and manipulation of polynucleotides, such as the Gateway® Cloning system (Invitrogen). As is known to those of skill in the art, expression vectors include elements that drive production of polypeptides of interest encoded by a polynucleotide in library host cells (e.g., promoter and other regulatory elements). In some embodiments, polypeptide expression is controlled by an inducible element (e.g., an inducible promoter, e.g., an IPTG- or arabinose-inducible promoter, or an IPTG-inducible phage T7 RNA polymerase system, a lactose (lac) promoter, a tryptophan (trp) promoter, a tac promoter, a trc promoter, a phage lambda promoter, an alkaline phosphatase (phoA) promoter, to give just a few examples; see Cantrell, Meth. in Mol. Biol., 235:257-276, Humana Press, Casali and Preston, Eds.). In some embodiments, polypeptides are expressed as cytoplasmic polypeptides. In some embodiments, the vector used for polypeptide expression is a vector that has a high copy number in a library host cell. In some embodiments, the vector used for expression has a copy number that is more than 25, 50, 75, 100, 150, 200, or 250 copies per cell. In some embodiments, the vector used for expression has a ColE1 origin of replication. Useful vectors for polypeptide expression in bacteria include pET vectors (Novagen), Gateway® pDEST vectors (Invitrogen), pGEX vectors (Amersham Biosciences), pPRO vectors (BD Biosciences), pBAD vectors (Invitrogen), pLEX vectors (Invitrogen), pMAL™ vectors (New England BioLabs), pGEMEX vectors (Promega), and pQE vectors (Qiagen). Vector systems for producing phage libraries are known and include Novagen T7Select® vectors, and New England Biolabs Ph.D.™ Peptide Display Cloning System.

In some embodiments, library host cells express (either constitutively, or when induced, depending on the selected expression system) a polypeptide of interest to at least 10%, 20%, 30%, 40%, 50%, 60%, or 70% of the total cellular protein. In some embodiments, the level a polypeptide available in or on a library member (e.g., cell, virus particle, liposome, bead) is such that antigen presenting cells exposed to a sufficient quantity of the library members are presented on MHC/HLA molecules polypeptide epitopes at a density that is comparable to the density presented by antigen presenting cells pulsed with purified peptides.

Methods for efficient, large-scale production of libraries are available. For example, site-specific recombinases or rare-cutting restriction enzymes can be used to transfer polynucleotides between expression vectors in the proper orientation and reading frame (Walhout et al., Meth. Enzymol. 328:575-592, 2000; Marsischky et al., Genome Res. 14:2020-202, 2004; Blommel et al., Protein Expr. Purif. 47:562-570, 2006).

For production of liposome libraries, expressed polypeptides (e.g., purified or partially purified polypeptides) can be entrapped in liposomal membranes, e.g., as described in Wassef et al., U.S. Pat. No. 4,863,874; Wheatley et al., U.S. Pat. No. 4,921,757; Huang et al., U.S. Pat. No. 4,925,661; or Martin et al., U.S. Pat. No. 5,225,212.

A library can be designed to include full-length polypeptides and/or portions of polypeptides. Expression of full-length polypeptides maximizes epitopes available for presentation by a human antigen presenting cell, thereby increasing the likelihood of identifying an antigen. However, in some embodiments, it is useful to express portions of polypeptides, or polypeptides that are otherwise altered, to achieve efficient expression. For example, in some embodiments, polynucleotides encoding polypeptides that are large (e.g., greater than 1,000 amino acids), that have extended hydrophobic regions, signal peptides, transmembrane domains, or domains that cause cellular toxicity, are modified (e.g., by C-terminal truncation, N-terminal truncation, or internal deletion) to reduce cytotoxicity and permit efficient expression a library cell, which in turn facilitates presentation of the encoded polypeptides on human cells. Other types of modifications, such as point mutations or codon optimization, may also be used to enhance expression.

The number of polypeptides included in a library can be varied. For example, in some embodiments, a library can be designed to express polypeptides from at least 5%, 10%, 15%, 20%, 25%, 35%, 40%, 45%, 50%, 55%, 60%, 70%, 75%, 80%, 85%, 90%, 95%, 97%, 98%, 99%, or more, of ORFs in a target cell (e.g., tumor cell). In some embodiments, a library expresses at least 10, 15, 20, 25, 30, 40, 50, 75, 100, 150, 200, 250, 300, 350, 400, 450, 500, 550, 600, 650, 700, 750, 800, 850, 900, 950, 1000, 2500, 5000, 10,000, or more different polypeptides of interest, each of which may represent a polypeptide encoded by a single full-length polynucleotide or portion thereof.

In some embodiments, assays may focus on identifying antigens that are secreted polypeptides, cell surface-expressed polypeptides, or virulence determinants, e.g., to identify antigens that are likely to be targets of both humoral and cell mediated immune responses.

In addition to polypeptides of interest, libraries can include tags or reporter proteins that allow one to easily purify, analyze, or evaluate MHC/HLA presentation, of the polypeptide of interest. In some embodiments, polypeptides expressed by a library include C-terminal tags that include both an MHC/HLA class I and an MHC/HLA class II-restricted T cell epitope from a model antigen, such as chicken ovalbumin (OVA). Library protein expression and MHC/HLA presentation is validated using these epitopes. In some embodiments, the epitopes are OVA₂₄₇₋₂₆₅ and OVA₂₅₈₋₂₆₅ respectfully, corresponding to positions in the amino acid sequence found in GenBank® under Acc. No. NP_990483. Expression and presentation of linked ORFs can be verified with antigen presentation assays using T cell hybridomas (e.g., B3Z T hybridoma cells, which are H2-K^{b} restricted, and KZO T hybridoma cells, which are H2-A^{k} restricted) that specifically recognize these epitopes.

Sets of library members (e.g., bacterial cells) can be provided on an array (e.g., on a solid support, such as a 96-well plate) and separated such that members in each location express a different polypeptide of interest, or a different set of polypeptides of interest.

Methods of using library members for identifying T cell antigens are described in detail below. In addition to these methods, library members also have utility in assays to identify B cell antigens. For example, lysate prepared from library members that include polypeptides of interest can be used to screen a sample comprising antibodies (e.g., a serum sample) from a subject (e.g., a subject who has been exposed to an infectious agent of interest, a subject who has cancer, and/or a control subject), to determine whether antibodies present in the subject react with the polypeptide of interest. Suitable methods for evaluating antibody reactivity are known and include, e.g., ELISA assays.

### Polypeptides of Interest

In some embodiments, methods and compositions described herein can be used to identify and/or detect immune responses to a polypeptide of interest. In some embodiments, a polypeptide of interest is encoded by an ORF from a target tumor cell, and members of a library include (e.g., internally express or carry) ORFs from a target tumor cell. In some such embodiments, a library can be used in methods described herein to assess immune responses to one or more polypeptides of interest encoded by one or more ORFs. In some embodiments, methods of the disclosure identify one or more polypeptides of interest as stimulatory antigens (e.g., that stimulate an immune response, e.g., a T cell response, e.g., expression and/or secretion of one or more immune mediators). In some embodiments, methods of the disclosure identify one or more polypeptides of interest as antigens or potential antigens that have minimal or no effect on an immune response (e.g., expression and/or secretion of one or more immune mediators). In some embodiments, methods of the disclosure identify one or more polypeptides of interest as inhibitory and/or suppressive antigens (e.g., that inhibit, suppress, down-regulate, impair, and/or prevent an immune response, e.g., a T cell response, e.g., expression and/or secretion of one or more immune mediators). In some embodiments, methods of the disclosure identify one or more polypeptides of interest as tumor antigens or potential tumor antigens, e.g., tumor specific antigens (TSAs, or neoantigens), tumor associated antigens (TAAs), or cancer/testis antigens (CTAs).

In some embodiments, a polypeptide of interest is a putative tumor antigen, and methods and compositions described herein can be used to identify and/or detect immune responses to one or more putative tumor antigens. For example, members of a library include (e.g., internally express or carry) putative tumor antigens (e.g., a polypeptide previously identified (e.g., by a third party) as a tumor antigen, e.g., identified as a tumor antigen using a method other than a method of the present disclosure). In some embodiments, a putative tumor antigen is a tumor antigen described herein. In some such embodiments, such libraries can be used to assess whether and/or the extent to which such putative tumor antigen mediates an immune response. In some embodiments, methods of the disclosure identify one or more putative tumor antigens as stimulatory antigens. In some embodiments, methods of the disclosure identify one or more putative tumor antigens as antigens that have minimal or no effect on an immune response. In some embodiments, methods of the disclosure identify one or more putative tumor antigens as inhibitory and/or suppressive antigens.

In some embodiments, a polypeptide of interest is a pre-selected tumor antigen, and methods and compositions described herein can be used to identify and/or detect immune responses to one or more pre-selected tumor antigens. For example, in some embodiments, members of a library include (e.g., internally express or carry) one or more polypeptides identified as tumor antigens using a method of the present disclosure and/or using a method other than a method of the present disclosure. In some such embodiments, such libraries can be used to assess whether and/or the extent to which such tumor antigens mediate an immune response by an immune cell from one or more subjects (e.g., a subject who has cancer and/or a control subject) to obtain one or more response profiles described herein. In some embodiments, methods of the disclosure identify one or more pre-selected tumor antigens as stimulatory antigens for one or more subjects. In some embodiments, methods of the disclosure identify one or more pre-selected tumor antigens as antigens that have minimal or no effect on an immune response for one or more subjects. In some embodiments, methods of the disclosure identify one or more pre-selected tumor antigens as inhibitory and/or suppressive antigens for one or more subjects.

In some embodiments, a polypeptide of interest is a known tumor antigen, and methods and compositions described herein can be used to identify and/or detect immune responses to one or more known tumor antigens. For example, in some embodiments, members of a library include (e.g., internally express or carry) one or more polypeptides identified as a tumor antigen using a method of the present disclosure and/or using a method other than a method of the present disclosure. In some such embodiments, such libraries can be used to assess whether and/or the extent to which such tumor antigens mediate an immune response by an immune cell from one or more subjects (e.g., a subject who has cancer and/or a control subject) to obtain one or more response profiles described herein. In some embodiments, methods of the disclosure identify one or more known tumor antigens as stimulatory antigens for one or more subjects. In some embodiments, methods of the disclosure identify one or more known tumor antigens as antigens that have minimal or no effect on an immune response for one or more subjects. In some embodiments, methods of the disclosure identify one or more known tumor antigens as inhibitory and/or suppressive antigens for one or more subjects.

In some embodiments, a polypeptide of interest is a potential tumor antigen, and methods and compositions described herein can be used to identify and/or detect immune responses to one or more potential tumor antigens. For example, in some embodiments, members of a library include (e.g., internally express or carry) one or more polypeptides identified as being of interest, e.g., encoding mutations associated with a tumor, using a method of the present disclosure and/or using a method other than a method of the present disclosure. In some such embodiments, such libraries can be used to assess whether and/or the extent to which such polypeptides mediate an immune response by an immune cell from one or more subjects (e.g., a subject who has cancer and/or a control subject) to obtain one or more response profiles described herein. In some embodiments, methods of the disclosure identify one or more polypeptides as stimulatory antigens for one or more subjects. In some embodiments, methods of the disclosure identify one or more polypeptides as antigens that have minimal or no effect on an immune response for one or more subjects. In some embodiments, methods of the disclosure identify one or more polypeptides as inhibitory and/or suppressive antigens for one or more subjects.

### Tumor Antigens

Polypeptides of interest used in methods and systems described herein include tumor antigens and potential tumor antigens, e.g., tumor specific antigens (TSAs, or neoantigens), tumor associated antigens (TAAs), and/or cancer/testis antigens (CTAs). Exemplary tumor antigens include, e.g., MART-1/MelanA (MART-I or MLANA), gp100 (Pmel 17 or SILV), tyrosinase, TRP-1, TRP-2, MAGE-1, MAGE-3 (also known as HIPS), BAGE, GAGE-1, GAGE-2, p15, Calcitonin, Calretinin, Carcinoembryonic antigen (CEA), Chromogranin, Cytokeratin, Desmin, Epithelial membrane protein (EMA), Factor VIII, Glial fibrillary acidic protein (GFAP), Gross cystic disease fluid protein (GCDFP-15), HMB-45, Human chorionic gonadotropin (hCG), inhibin, lymphocyte marker, MART-1 (Melan-A), Myo D1, muscle-specific actin (MSA), neurofilament, neuron-specific enolase (NSE), placental alkaline phosphatase (PLAP), prostate-specific antigen, PTPRC (CD45), S100 protein, smooth muscle actin (SMA), synaptophysin, thyroglobulin, thyroid transcription factor-1, Tumor M2-PK, vimentin, p53, Ras, HER-2/neu, BCR-ABL, E2A-PRL, H4-RET, IGH-IGK, MYL-RAR, Epstein Barr virus antigens (e.g., EBNA1), human papillomavirus (HPV) antigen E6 or E7 (HPV_E6 or HPV_E7), TSP-180, MAGE-4, MAGE-5, MAGE-6, RAGE, NY-ESO-1 (also known as CTAG1B), erbB, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein (AFP), beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, MUC16, IL13Ra2, FRa, VEGFR2, Lewis Y, FAP, EphA2, CEACAM5, EGFR, CA6, CA9, GPNMB, EGP1, FOLR1, endothelial receptor, STEAP1, SLC44A4, Nectin-4, AGS-16, guanalyl cyclase C, MUC-1, CFC1B, integrin alpha 3 chain (of a3b1, a laminin receptor chain), TPS, CD19, CD20, CD22, CD30, CD31, CD72, CD180, CD171 (L1CAM), CD123, CD133, CD138, CD37, CD70, CD79a, CD79b, CD56, CD74, CD166, CD71, CD34, CD99, CD117, CD80, CD28, CD13, CD15, CD25, CD10, CLL-1/CLEC12A, ROR1, Glypican 3 (GPC3), Mesothelin, CD33/IL3Ra, c-Met, PSCA, PSMA, Glycolipid F77, EGFRvIII, BCMA, GD-2, PSAP, prostein (also known as P501S), PSMA, Survivin (also known as BIRC5), and MAGE-A3, MAGEA2, MAGEA4, MAGEA6, MAGEA9, MAGEA10, MAGEA12, BIRC5, CDH3, CEACAM3, CGB_isoform2, ELK4, ERBB2, HPSE1, HPSE2, KRAS_isoform1, KRAS_isoform2, MUC1, SMAD4, TERT.2, TERT.3, TGFBR2, EGAG9_isoform1, TP53, CGB_isoform1, IMPDH2, LCK, angiopoietin-1 (Ang1) (also known as ANGPT1), XIAP (also known as BIRC4), galectin-3 (also known as LGALS3), VEGF-A (also known as VEGF), ATP6S1 (also known as ATP6AP1), MAGE-A1, cIAP-1 (also known as BIRC2), macrophage migration inhibitory factor (MIF), galectin-9 (also known as LGALS9), progranulin PGRN (also known as granulin), OGFR, MLIAP (also known as BIRC7), TBX4 (also known as ICPPS, SPS or T-Box4), secretory leukocyte protein inhibitor (Slpi) (also known as antileukoproteinase), Ang2 (also known as ANGPT2), galectin-1 (also known as LGALS1), TRP-2 (also known as DCT), hTERT (telomerase reverse transcriptase) tyrosinase-related protein 1 (TRP-1, TYRP1), NOR-90/UBF-2 (also known as UBTF), LGMN, SPA17, PRTN3, TRRAP_1, TRRAP_2, TRRAP_3, TRRAP_4, MAGEC2, PRAME, SOX10, RAC1, HRAS, GAGE4, AR, CYP1B1, MMP8, TYR, PDGFRB, KLK3, PAX3, PAX5, ST3GAL5, PLAC1, RhoC, MYCN, REG3A, CSAG2, CTAG2-1a, CTAG2-1b, PAGE4, BRAF, GRM3, ERBB4, KIT, MAPK1, MFI2, SART3, ST8SIA1, WDR46, AKAP-4, RGS5, FOSL1, PRM2, ACRBP, CTCFL, CSPG4, CCNB1, MSLN, WT1, SSX2, KDR, ANKRD30A, MAGED1, MAP3K9, XAGE1B, PREX2, CD276, TEK, AIM1, ALK, FOLH1, GRIN2A MAP3K5 and one or more isoforms of any preceding tumor antigens. Exemplary tumor antigens are provided in the accompanying list of sequences. In some embodiments, a tumor antigen comprises a variant of an amino acid sequence provided in the accompanying list of sequences (e.g., a sequence that is at least about 85%, 90%, 95%, 96%, 97% 98%, 99% identical to an amino acid sequence provided in the accompanying list of sequences and/or a sequence that includes a mutation, deletion, and/or insertion of at least one amino acid (e.g., 1, 2, 3, 4, 5, 6, 7, 8, 9, 10, or more amino acids) relative to an amino acid sequence provided in the accompanying list of sequences).

Tumor specific antigens (TSAs, or neoantigens) are tumor antigens that are not encoded in normal host genome (see, e.g., Yarchoan et al., Nat. Rev. Cancer. 2017 Feb. 24. doi: 10.1038/nrc.2016.154; Gubin et al., J. Clin. Invest. 125:3413-3421 (2015)). In some embodiments, TSAs arise from somatic mutations and/or other genetic alterations. In some embodiments, TSAs arise from missense or in-frame mutations. In some embodiments, TSAs arise from frame-shift mutations or loss-of-stop-codon mutations. In some embodiments, TSAs arise from insertion or deletion mutations. In some embodiments, TSAs arise from duplication or repeat expansion mutations. In some embodiments, TSAs arise from splice variants or improper splicing. In some embodiments, TSAs arise from gene fusions. In some embodiments, TSAs arise from translocations. In some embodiments, TSAs arise from post-translational peptide splicing (i.e., are not encoded). In some embodiments, TSAs include oncogenic viral proteins. For example, as with Merkel cell carcinoma (MCC) associated with the Merkel cell polyomavirus (MCPyV) and cancers of the cervix, oropharynx and other sites associated with the human papillomavirus (HPV), TSAs include proteins encoded by viral open reading frames. For purposes of this disclosure, the terms “mutation” and “mutations” encompass all mutations and genetic alterations that may give rise to an antigen, i.e. encoded in the genome or otherwise present, in a cancer or tumor cell of a subject, but not in a normal or non-cancerous cell of the same subject. In some embodiments, TSAs are specific (personal) to a subject. In some embodiments, TSAs are shared by more than one subject, e.g., less than 1%, 1-3%, 1-5%, 1-10%, or more of subjects suffering from a cancer. In some embodiments, TSAs are shared by a cohort of subjects suffering from a cancer. In some embodiments, TSAs shared by more than one subject or by a cohort of subjects may be known or pre-selected.

In some embodiments, a TSA is encoded by an open reading frame from a virus e.g, an oncovirus. For example, a library can be designed to express polypeptides from one of the following viruses: an immunodeficiency virus (e.g., a human immunodeficiency virus (HIV), e.g., HIV-1, HIV-2), a hepatitis virus (e.g., hepatitis B virus (HBV), hepatitis C virus (HCV), hepatitis A virus, non-A and non-B hepatitis virus), a herpes virus (e.g., herpes simplex virus type I (HSV-1), HSV-2, Varicella-zoster virus, Epstein Barr virus, human cytomegalovirus, human herpesvirus 6 (HHV-6), HHV-7, HHV-8, Kaposi's sarcoma-associated herpesvirus), a poxvirus (e.g., variola, vaccinia, monkeypox, Molluscum contagiosum virus), an influenza virus, a human papilloma virus (HPV), Merkel cell polyoma virus, human T-lymphotropic virus 1, adenovirus, rhinovirus, coronavirus, respiratory syncytial virus, rabies virus, coxsackie virus, human T cell leukemia virus (types I, II and III), parainfluenza virus, paramyxovirus, poliovirus, rotavirus, rhinovirus, rubella virus, measles virus, mumps virus, adenovirus, yellow fever virus, Norwalk virus, West Nile virus, a Dengue virus, Severe Acute Respiratory Syndrome Coronavirus (SARS-CoV), bunyavirus, Ebola virus, Marburg virus, Eastern equine encephalitis virus, Venezuelan equine encephalitis virus, Japanese encephalitis virus, St. Louis encephalitis virus, Junin virus, Lassa virus, and Lymphocytic choriomeningitis virus. Libraries for other viruses can also be produced and used according to methods described herein.

Tumor specific antigens are known in the art, any of which can be used in methods described herein. In some embodiments, gene sequences encoding polypeptides that are potential or putative neoantigens are determined by sequencing the genome and/or exome of tumor tissue and healthy tissue from a subject having cancer using next generation sequencing technologies. In some embodiments, genes that are selected based on their frequency of mutation and ability to encode a potential or putative neoantigen are sequenced using next-generation sequencing technology. Next-generation sequencing applies to genome sequencing, genome resequencing, transcriptome profiling (RNA-Seq), DNA-protein interactions (ChIP-sequencing), and epigenome characterization (de Magalhaes et al., (2010) Ageing Research Reviews 9 (3): 315-323; Hall N (2007) J. Exp. Biol. 209 (Pt 9): 1518-1525; Church, (2006) Sci. Am. 294 (1): 46-54; ten Bosch et al., (2008) Journal of Molecular Diagnostics 10 (6): 484-492; Tucker T et al., (2009) The American Journal of Human Genetics 85 (2): 142-154). Next-generation sequencing can be used to rapidly reveal the presence of discrete mutations such as coding mutations in individual tumors, e.g., single amino acid changes (e.g., missense mutations, in-frame mutations) or novel stretches of amino acids generated by frame-shift insertions, deletions, gene fusions, read-through mutations in stop codons, duplication or repeat expansion mutations, and translation of splice variants or improperly spliced introns, and translocations (e.g., “neoORFs”).

Another method for identifying potential or putative neoantigens is direct protein sequencing. Protein sequencing of enzymatic digests using multidimensional MS techniques (MSn) including tandem mass spectrometry (MS/MS) can also be used to identify neoantigens. Such proteomic approaches can be used for rapid, highly automated analysis (see, e.g., Gevaert et al., Electrophoresis 21:1145-1154 (2000)). High-throughput methods for de novo sequencing of unknown proteins can also be used to analyze the proteome of a subject's tumor to identify expressed potential or putative neoantigens. For example, meta shotgun protein sequencing may be used to identify expressed potential or putative neoantigens (see e.g., Guthals et al., (2012) Molecular and Cellular Proteomics 11(10):1084-96).

Potential or putative neoantigens may also be identified using MHC/HLA multimers to identify neoantigen-specific T cell responses. For example, high-throughput analysis of neoantigen-specific T cell responses in patient samples may be performed using MHC/HLA tetramer-based screening techniques (see e.g., Hombrink et al., (2011) PLoS One; 6(8): e22523; Hadrup et al., (2009) Nature Methods, 6(7):520-26; van Rooij et al., (2013) Journal of Clinical Oncology, 31:1-4; and Heemskerk et al., (2013) EMBO Journal, 32(2):194-203).

In some embodiments, one or more known or pre-selected tumor specific antigens, or one or more potential or putative tumor specific antigens identified using one of these methods, can be included in a library described herein.

Tumor associated antigens (TAAs) include proteins encoded in a normal genome (see, e.g., Ward et al., Adv. Immunol. 130:25-74 (2016)). In some embodiments, TAAs are either normal differentiation antigens or aberrantly expressed normal proteins. Overexpressed normal proteins that possess growth/survival-promoting functions, such as Wilms tumor 1 (WT1) (Ohminami et al., Blood 95:286-293 (2000)) or Her2/neu (Kawashima et al., Cancer Res. 59:431-435 (1999)), are TAAs that directly participate in the oncogenic process. Post-translational modifications, such as phosphorylation, of proteins may also lead to formation of TAAs (Doyle, J. Biol. Chem. 281:32676-32683 (2006); Cobbold, Sci. Transl. Med. 5:203ra125 (2013)). TAAs are generally shared by more than one subject, e.g., less than 1%, 1-3%, 1-5%, 1-10%, 1-20%, or more of subjects suffering from a cancer. In some embodiments, TAAs are known or pre-selected tumor antigens. In some embodiments, with respect to an individual subject, TAAs are potential or putative tumor antigens. Cancer/testis antigens (CTAs) are expressed by various tumor types and by reproductive tissues (for example, testes, fetal ovaries and trophoblasts) but have limited or no detectable expression in other normal tissues in the adult and are generally not presented on normal reproductive cells, because these tissues do not express HLA class I molecules (see, e.g., Coulie et al., Nat. Rev. Cancer 14:135-146 (2014); Simpson et al., Nat. Rev. Cancer 5:615-625 (2005); Scanlan et al., Immunol. Rev. 188:22-32 (2002)).

### Library Screens

### Human Cells for Antigen Presentation

The present disclosure provides, inter alia, compositions and methods for identifying tumor antigens recognized by human immune cells. Human antigen presenting cells express ligands for antigen receptors and other immune activation molecules on human lymphocytes. Given differences in HLA peptide binding specificities and antigen processing enzymes between species, antigens processed and presented by human cells are more likely to be physiologically relevant human antigens in vivo than antigens identified in non-human systems. Accordingly, methods of identifying these antigens employ human cells to present candidate tumor antigen polypeptides. Any human cell that internalizes library members and presents polypeptides expressed by the library members on HLA molecules can be used as an antigen presenting cell according to the present disclosure. In some embodiments, human cells used for antigen presentation are primary human cells. The cells can include peripheral blood mononuclear cells (PBMC) of a human. In some embodiments, peripheral blood cells are separated into subsets (e.g., subsets comprising dendritic cells, macrophages, monocytes, B cells, or combinations thereof) prior to use in an antigen presentation assay. In some embodiments, a subset of cells that expresses HLA class II is selected from peripheral blood. In one example, a cell population including dendritic cells is isolated from peripheral blood. In some embodiments, a subset of dendritic cells is isolated (e.g., plasmacytoid, myeloid, or a subset thereof). Human dendritic cell markers include CD1c, CD1a, CD303, CD304, CD141, and CD209. Cells can be selected based on expression of one or more of these markers (e.g., cells that express CD303, CD1c, and CD141).

Dendritic cells can be isolated by positive selection from peripheral blood using commercially available kits (e.g., from Miltenyi Biotec Inc.). In some embodiments, the dendritic cells are expanded ex vivo prior to use in an assay. Dendritic cells can also be produced by culturing peripheral blood cells under conditions that promote differentiation of monocyte precursors into dendritic cells in vitro. These conditions typically include culturing the cells in the presence of cytokines such as GM-CSF and IL-4 (see, e.g., Inaba et al., Isolation of dendritic cells, Curr. Protoc. Immunol. May; Chapter 3: Unit 3.7, 2001). Procedures for in vitro expansion of hematopoietic stem and progenitor cells (e.g., taken from bone marrow or peripheral blood), and differentiation of these cells into dendritic cells in vitro, is described in U.S. Pat. No. 5,199,942, and U.S. Pat. Pub. 20030077263. Briefly, CD34⁺ hematopoietic stem and progenitor cells are isolated from peripheral blood or bone marrow and expanded in vitro in culture conditions that include one or more of Flt3-L, IL-1, IL-3, and c-kit ligand.

In some embodiments, immortalized cells that express human MHC molecules (e.g., human cells, or non-human cells that are engineered to express HLA molecules) are used for antigen presentation. For example, assays can employ COS cells transfected with HLA molecules or HeLa cells.

In some embodiments, both the antigen presenting cells and immune cells used in the method are derived from the same subject (e.g., autologous T cells and APC are used). In these embodiments, it can be advantageous to sequentially isolate subsets of cells from peripheral blood of the subject, to maximize the yield of cells available for assays. For example, one can first isolate CD4⁺ and CD8⁺ T cell subsets from the peripheral blood. Next, dendritic cells (DC) are isolated from the T cell-depleted cell population. The remaining T- and DC-depleted cells are used to supplement the DC in assays, or are used alone as antigen presenting cells. In some embodiments, DC are used with T- and DC-depleted cells in an assay, at a ratio of 1:2, 1:3, 1:4, or 1:5. In some embodiments, the antigen presenting cells and immune cells used in the method are derived from different subjects (e.g., heterologous T cells and APC are used).

Antigen presenting cells can be isolated from sources other than peripheral blood. For example, antigen presenting cells can be taken from a mucosal tissue (e.g., nose, mouth, bronchial tissue, tracheal tissue, the gastrointestinal tract, the genital tract (e.g., vaginal tissue), or associated lymphoid tissue), peritoneal cavity, lymph nodes, spleen, bone marrow, thymus, lung, liver, kidney, neuronal tissue, endocrine tissue, or other tissue, for use in screening assays. In some embodiments, cells are taken from a tissue that is the site of an active immune response (e.g., an ulcer, sore, or abscess). Cells may be isolated from tissue removed surgically, via lavage, or other means.

Antigen presenting cells useful in methods described herein are not limited to “professional” antigen presenting cells. In some embodiments, non-professional antigen presenting cells can be utilized effectively in the practice of methods of the present disclosure. Non-professional antigen presenting cells include fibroblasts, epithelial cells, endothelial cells, neuronal/glial cells, lymphoid or myeloid cells that are not professional antigen presenting cells (e.g., T cells, neutrophils), muscle cells, liver cells, and other types of cells.

Antigen presenting cells are cultured with library members that express a polypeptide of interest (and, if desired, a cytolysin polypeptide) under conditions in which the antigen presenting cells internalize, process and present polypeptides expressed by the library members on MHC/HLA molecules. In some embodiments, library members are killed or inactivated prior to culture with the antigen presenting cells. Cells or viruses can be inactivated by any appropriate agent (e.g., fixation with organic solvents, irradiation, freezing). In some embodiments, the library members are cells that express ORFs linked to a tag (e.g., a tag which comprises one or more known T cell epitopes) or reporter protein, expression of which has been verified prior to the culturing.

In some embodiments, antigen presenting cells are incubated with library members at 37° C. for between 30 minutes and 5 hours (e.g., for 45 min. to 1.5 hours). After the incubation, the antigen presenting cells can be washed to remove library members that have not been internalized. In certain embodiments, the antigen presenting cells are non-adherent, and washing requires centrifugation of the cells. The washed antigen presenting cells can be incubated at 37° C. for an additional period of time (e.g., 30 min. to 2 hours) prior to exposure to lymphocytes, to allow antigen processing. In some embodiments, it is desirable to fix and kill the antigen presenting cells prior to exposure to lymphocytes (e.g., by treating the cells with 1% paraformaldehyde).

The antigen presenting cell and library member numbers can be varied, so long as the library members provide quantities of polypeptides of interest sufficient for presentation on MHC/HLA molecules. In some embodiments, antigen presenting cells are provided in an array, and are contacted with sets of library cells, each set expressing a different polypeptide of interest. In certain embodiments, each location in the array includes 1×10³-1×10⁶ antigen presenting cells, and the cells are contacted with 1×10³-1×10⁸ library cells which are bacterial cells.

In any of the embodiments described herein, antigen presenting cells can be freshly isolated, maintained in culture, and/or thawed from frozen storage prior to incubation with library cells, or after incubation with library cells.

### Human Lymphocytes

In methods of the present disclosure, human lymphocytes are tested for antigen-specific reactivity to antigen presenting cells, e.g., antigen presenting cells that have been incubated with libraries expressing polypeptides of interest as described above. The methods of the present disclosure permit rapid identification of human antigens using pools of lymphocytes isolated from an individual, or progeny of the cells. The detection of antigen-specific responses does not rely on laborious procedures to isolate individual T cell clones. In some embodiments, the human lymphocytes are primary lymphocytes. In some embodiments, human lymphocytes are NKT cells, gamma-delta T cells, or NK cells. Just as antigen presenting cells may be separated into subsets prior to use in antigen presentation assays, a population of lymphocytes having a specific marker or other feature can be used. In some embodiments, a population of T lymphocytes is isolated. In some embodiments, a population of CD4⁺ T cells is isolated. In some embodiments, a population of CD8⁺ T cells is isolated. CD8⁺ T cells recognize peptide antigens presented in the context of MHC/HLA class I molecules. Thus, in some embodiments, the CD8⁺ T cells are used with antigen presenting cells that have been exposed to library host cells that co-express a cytolysin polypeptide, in addition to a polypeptide of interest. T cell subsets that express other cell surface markers may also be isolated, e.g., to provide cells having a particular phenotype. These include CLA (for skin-homing T cells), CD25, CD30, CD69, CD154 (for activated T cells), CD45RO (for memory T cells), CD294 (for Th2 cells), γ/δ TCR-expressing cells, CD3 and CD56 (for NK T cells). Other subsets can also be selected.

Lymphocytes can be isolated, and separated, by any means known in the art (e.g., using antibody-based methods such as those that employ magnetic bead separation, panning, or flow cytometry). Reagents to identify and isolate human lymphocytes and subsets thereof are well known and commercially available.

Lymphocytes for use in methods described herein can be isolated from peripheral blood mononuclear cells, or from other tissues in a human. In some embodiments, lymphocytes are taken from tumors, lymph nodes, a mucosal tissue (e.g., nose, mouth, bronchial tissue, tracheal tissue, the gastrointestinal tract, the genital tract (e.g., vaginal tissue), or associated lymphoid tissue), peritoneal cavity, spleen, thymus, lung, liver, kidney, neuronal tissue, endocrine tissue, peritoneal cavity, bone marrow, or other tissues. In some embodiments, cells are taken from a tissue that is the site of an active immune response (e.g., an ulcer, sore, or abscess). Cells may be isolated from tissue removed surgically, via lavage, or other means.

Lymphocytes taken from an individual can be maintained in culture or frozen until use in antigen presentation assays. In some embodiments, freshly isolated lymphocytes can be stimulated in vitro by antigen presenting cells exposed to library cells as described above. In some embodiments, these lymphocytes exhibit detectable stimulation without the need for prior non-antigen specific expansion. However, primary lymphocytes also elicit detectable antigen-specific responses when first stimulated non-specifically in vitro. Thus, in some embodiments, lymphocytes are stimulated to proliferate in vitro in a non-antigen specific manner, prior to use in an antigen presentation assay. Lymphocytes can also be stimulated in an antigen-specific manner prior to use in an antigen presentation assay. In some embodiments, cells are stimulated to proliferate by a library (e.g., prior to use in an antigen presentation assay that employs the library). Expanding cells in vitro provides greater numbers of cells for use in assays. Primary T cells can be stimulated to expand, e.g., by exposure to a polyclonal T cell mitogen, such as phytohemagglutinin or concanavalin, by treatment with antibodies that stimulate proliferation, or by treatment with particles coated with the antibodies. In some embodiments, T cells are expanded by treatment with anti-CD2, anti-CD3, and anti-CD28 antibodies. In some embodiments, T cells are expanded by treatment with interleukin-2 (IL-2). In some embodiments, lymphocytes are thawed from frozen storage and expanded (e.g., stimulated to proliferate, e.g., in a non-antigen specific manner or in an antigen-specific manner) prior to contacting with antigen presenting cells. In some embodiments, lymphocytes are thawed from frozen storage and are not expanded prior to contacting with antigen presenting cells. In some embodiments, lymphocytes are freshly isolated and expanded (e.g., stimulated to proliferate, e.g., in a non-antigen specific manner or in an antigen-specific manner) prior to contacting with antigen presenting cells.

### Antigen Presentation Assays

In antigen presentation assays, T cells are cultured with antigen presenting cells prepared according to the methods described above, under conditions that permit T cell recognition of peptides presented by MHC/HLA molecules on the antigen presenting cells. In some embodiments, T cells are incubated with antigen presenting cells at 37° C. for between 12-48 hours (e.g., for 24 hours). In some embodiments, T cells are incubated with antigen presenting cells at 37° C. for 3, 4, 5, 6, 7, or 8 days. Numbers of antigen presenting cells and T cells can be varied. In some embodiments, the ratio of T cells to antigen presenting cells in a given assay is 1:10, 1:5, 1:2, 1:1, 2:1, 5:1, 10:1, 20:1, 25:1, 30:1, 32:1, 35:1 or 40:1. In some embodiments, antigen presenting cells are provided in an array (e.g., in a 96-well plate), wherein cells in each location of the array have been contacted with sets of library cells, each set including a different polypeptide of interest. In certain embodiments, each location in the array includes 1×10³-1×10⁶ antigen presenting cells, and the cells are contacted with 1×10³-1×10⁶ T cells.

After T cells have been incubated with antigen presenting cells, cultures are assayed for activation. Lymphocyte activation can be detected by any means known in the art, e.g., T cell proliferation, phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers. In some embodiments, culture supernatants are harvested and assayed for increased and/or decreased expression and/or secretion of one or more polypeptides associated with activation, e.g., a cytokine, soluble mediator, cell surface marker, or other immune mediator. In some embodiments, the one or more cytokines are selected from TRAIL, IFN-gamma, IL-12p70, IL-2, TNF-alpha, MIP1-alpha, MIP1-beta, CXCL9, CXCL10, MCP1, RANTES, IL-1 beta, IL-4, IL-6, IL-8, IL-9, IL-10, IL-13, IL-15, CXCL11, IL-3, IL-5, IL-17, IL-18, IL-21, IL-22, IL-23A, IL-24, IL-27, IL-31, IL-32, TGF-beta, CSF, GM-CSF, TRANCE (also known as RANKL), MIP3-alpha, and fractalkine. In some embodiments, the one or more soluble mediators are selected from granzyme A, granzyme B, granzyme K, sFas, sFasL, perforin, and granulysin. In some embodiments, the one or more cell surface markers are selected from CD107a, CD107b, CD25 (IL-2RA), CD69, CD45RA, CD45RO, CD137 (4-1BB), CD44, CD62L, CD27, CCR7, CD154 (CD40L), KLRG-1, CD71, HLA-DR, CD122 (IL-2RB), CD28, IL7Ra (CD127), CD38, CD26, CD134 (OX-40), CTLA-4 (CD152), LAG-3, TIM-3 (CD366), CD39, PD1 (CD279), FoxP3, TIGIT, CD160, BTLA, 2B4 (CD244), CCR2, CCR5, CX3CR1, NKG2D, CD39, KLRD1, LGALS1 (encoding Galectin-1), and KLRG1. Cytokine secretion in culture supernatants can be detected, e.g., by ELISA, bead array, e.g., with a Luminex® analyzer. Cytokine production can also be assayed by RT-PCR of mRNA isolated from the T cells, or by ELISPOT analysis of cytokines released by the T cells. In some embodiments, proliferation of T cells in the cultures is determined (e.g., by detecting ³H thymidine incorporation). In some embodiments, target cell lysis is determined (e.g., by detecting T cell dependent lysis of antigen presenting cells labeled with Na₂⁵¹CrO₄). Target cell lysis assays are typically performed with CD8⁺ T cells. Protocols for these detection methods are known. See, e.g., Current Protocols In Immunology, John E. Coligan et al. (eds), Wiley and Sons, New York, N.Y., 2007. One of skill in the art understands that appropriate controls are used in these detection methods, e.g., to adjust for non-antigen specific background activation, to confirm the presenting capacity of antigen presenting cells, and to confirm the viability of lymphocytes.

In some embodiments, antigen presenting cells and lymphocytes used in the method are from the same individual. In some embodiments, antigen presenting cells and lymphocytes used in the method are from different individuals.

In some embodiments, antigen presentation assays are repeated using lymphocytes from the same individual that have undergone one or more previous rounds of exposure to antigen presenting cells, e.g., to enhance detection of responses, or to enhance weak initial responses. In some embodiments, antigen presentation assays are repeated using antigen presenting cells from the same individual that have undergone one or more previous rounds of exposure to a library, e.g., to enhance detection of responses, or to enhance weak initial responses. In some embodiments, antigen presentation assays are repeated using lymphocytes from the same individual that have undergone one or more previous rounds of exposure to antigen presenting cells, and antigen presenting cells from the same individual that have undergone one or more previous rounds of exposure to a library, e.g., to enhance detection of responses, or to enhance weak initial responses. In some embodiments, antigen presentation assays are repeated using antigen presenting cells and lymphocytes from different individuals, e.g., to identify antigens recognized by multiple individuals, or compare reactivities that differ between individuals.

### Methods of Identifying Tumor Antigens

One advantage of methods described herein is their ability to identify clinically relevant human antigens. Humans that have cancer may have lymphocytes that specifically recognize tumor antigens, which are the product of an adaptive immune response arising from prior exposure. In some embodiments, these cells are present at a higher frequency than cells from an individual who does not have cancer, and/or the cells are readily reactivated when re-exposed to the proper antigenic stimulus (e.g., the cells are “memory” cells). Thus, humans that have or have had cancer are particularly useful donors of cells for identifying antigens in vitro. The individual may be one who has recovered from cancer. In some embodiments, the individual has been recently diagnosed with cancer (e.g., the individual was diagnosed less than one year, three months, two months, one month, or two weeks, prior to isolation of lymphocytes and/or antigen presenting cells from the individual). In some embodiments, the individual was first diagnosed with cancer more than three months, six months, or one year prior to isolation of lymphocytes and/or antigen presenting cells.

In some embodiments, lymphocytes are screened against antigen presenting cells that have been contacted with a library of cells whose members express or carry polypeptides of interest, and the lymphocytes are from an individual who has not been diagnosed with cancer. In some embodiments, such lymphocytes are used to determine background (i.e., non-antigen-specific) reactivities. In some embodiments, such lymphocytes are used to identify antigens, reactivity to which exists in non-cancer individuals.

Cells from multiple donors (e.g., multiple subjects who have cancer) can be collected and assayed in methods described herein. In some embodiments, cells from multiple donors are assayed in order to determine if a given tumor antigen is reactive in a broad portion of the population, or to identify multiple tumor antigens that can be later combined to produce an immunogenic composition that will be effective in a broad portion of the population.

Antigen presentation assays are useful in the context of both infectious and non-infectious diseases. The methods described herein are applicable to any context in which a rapid evaluation of human cellular immunity is beneficial. In some embodiments, antigenic reactivity to polypeptides that are differentially expressed by neoplastic cells (e.g., tumor cells) is evaluated. Sets of nucleic acids differentially expressed by neoplastic cells have been identified using established techniques such as subtractive hybridization. Methods described herein can be used to identify antigens that were functional in a subject in which an anti-tumor immune response occurred. In other embodiments, methods are used to evaluate whether a subject has lymphocytes that react to a tumor antigen or set of tumor antigens.

In some embodiments, antigen presentation assays are used to examine reactivity to autoantigens in cells of an individual, e.g., an individual predisposed to, or suffering from, an autoimmune condition. Such methods can be used to provide diagnostic or prognostic indicators of the individual's disease state, or to identify autoantigens. For these assays, in some embodiments, libraries that include an array of human polypeptides are prepared. In some embodiments, libraries that include polypeptides from infectious agents which are suspected of eliciting cross-reactive responses to autoantigens are prepared. For examples of antigens from infectious agents thought to elicit cross-reactive autoimmune responses, see Barzilai et al., Curr Opin Rheumatol., 19(6):636-43, 2007; Ayada et al., Ann NY Acad Sci., 1108:594-602, 2007; Drouin et al., Mol Immunol., 45(1):180-9, 2008; and Bach, J Autoimmun., 25 Supp1:74-80, 2005.

As discussed, the present disclosure includes methods in which polypeptides of interest are included in a library (e.g., expressed in library cells or carried in or on particles or beads). After members of the library are internalized by antigen presenting cells, the polypeptides of interest are proteolytically processed within the antigen presenting cells, and peptide fragments of the polypeptides are presented on MHC/HLA molecules expressed in the antigen presenting cells. The identity of the polypeptide that stimulates a human lymphocyte in an assay described herein can be determined from examination of the set of library cells that were provided to the antigen presenting cells that produced the stimulation. In some embodiments, it is useful to map the epitope within the polypeptide that is bound by MHC/HLA molecules to produce the observed stimulation. This epitope, or the longer polypeptide from which it is derived (both of which are referred to as an “antigen” herein) can form the basis for an immunogenic composition, or for an antigenic stimulus in future antigen presentation assays.

Methods for identifying peptides bound by MHC/HLA molecules are known. In some embodiments, epitopes are identified by generating deletion mutants of the polypeptide of interest and testing these for the ability to stimulate lymphocytes. Deletions that lose the ability to stimulate lymphocytes, when processed and presented by antigen presenting cells, have lost the peptide epitope. In some embodiments, epitopes are identified by synthesizing peptides corresponding to portions of the polypeptide of interest and testing the peptides for the ability to stimulate lymphocytes (e.g., in antigen presentation assays in which antigen presenting cells are pulsed with the peptides). Other methods for identifying MHC/HLA-bound peptides involve lysis of the antigen presenting cells that include the antigenic peptide, affinity purification of the MHC/HLA molecules from cell lysates, and subsequent elution and analysis of peptides from the MHC/HLA (Falk, K. et al., Nature 351:290, 1991, and U.S. Pat. No. 5,989,565).

In other embodiments, it is useful to identify the clonal T cell receptors that have been expanded in response to the antigen. Clonal T cell receptors are identified by DNA sequencing of the T cell receptor repertoire (Howie et al, 2015 Sci Trans Med 7:301). TCRs of known specificity and function, can be transfected into other cell types and used in functional studies or for novel immunotherapies.

In other embodiments, it is useful to identify and isolate T cells responsive to a tumor antigen in a subject. The isolated T cells can be expanded ex vivo and administered to a subject for cancer therapy or prophylaxis.

### Methods of Identifying Immune Responses of a Subject

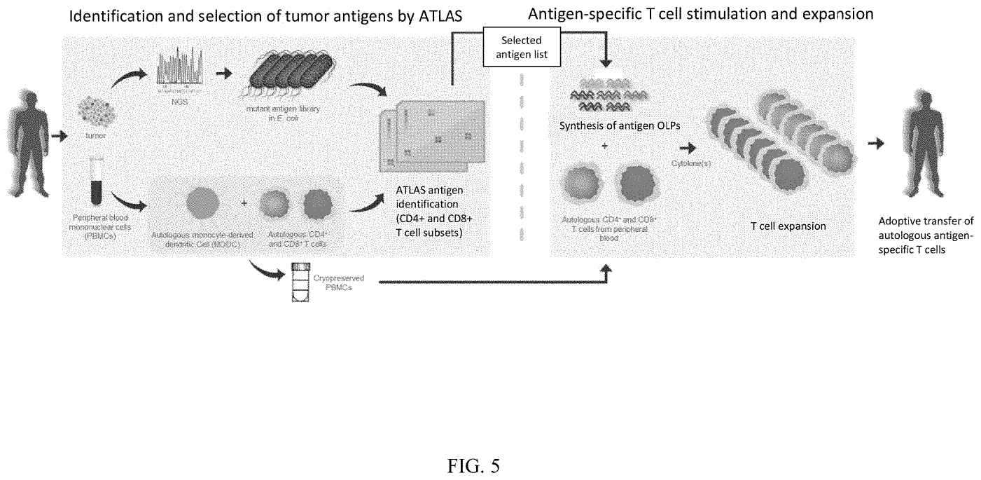

The disclosure provides methods of identifying one or more immune responses of a subject. One exemplary method of identifying tumor antigens is depicted schematically in the left portion of FIG. 5. In some embodiments, one or more immune responses of a subject are determined by a) providing a library described herein that includes a panel of tumor antigens (e.g., known tumor antigens, tumor antigens described herein, or tumor antigens, potential tumor antigens, and/or other polypeptides of interest identified using a method described herein); b) contacting the library with antigen presenting cells from the subject; c) contacting the antigen presenting cells with lymphocytes from the subject; and d) determining whether one or more lymphocytes are stimulated by, inhibited and/or suppressed by, activated by, or non-responsive to one or more tumor antigens presented by one or more antigen presenting cells. In some embodiments, the library includes about 1, 3, 5, 10, 15, 20, 25, 30, 40, 50, 60, 70, 80, 90, 100, or more tumor antigens.

In some embodiments, lymphocyte stimulation, non-stimulation, inhibition and/or suppression, activation, and/or non-responsiveness is determined by assessing levels of one or more expressed or secreted cytokines or other immune mediators described herein. In some embodiments, levels of one or more expressed or secreted cytokines that is at least 20%, 40%, 60%, 80%, 100%, 120%, 140%, 160%, 180%, 200% or more, higher than a control level indicates lymphocyte stimulation. In some embodiments, a level of one or more expressed or secreted cytokines that is at least 1, 2, 3, 4, or 5 standard deviations greater than the mean of a control level indicates lymphocyte stimulation. In some embodiments, a level of one or more expressed or secreted cytokines that is at least 1, 2, 3, 4 or 5 median absolute deviations (MADs) greater than a median response level to a control indicates lymphocyte stimulation. In some embodiments, a control is a negative control, for example, a clone expressing Neon Green (NG).

In some embodiments, a level of one or more expressed or secreted cytokines that is at least 20%, 40%, 60%, 80%, 100%, 120%, 140%, 160%, 180%, 200% or more, lower than a control level indicates lymphocyte inhibition and/or suppression. In some embodiments, a level of one or more expressed or secreted cytokines that is at least 1, 2, 3, 4 or 5 standard deviations lower than the mean of a control level indicates lymphocyte inhibition and/or suppression. In some embodiments, a level of one or more expressed or secreted cytokines that is at least 1, 2, 3, 4 or 5 median absolute deviations (MADs) lower than a median response level to a control indicates lymphocyte inhibition and/or suppression. In some embodiments, a control is a negative control, for example, a clone expressing Neon Green (NG).

In some embodiments, levels of one or more expressed or secreted cytokines that is at least 20%, 40%, 60%, 80%, 100%, 120%, 140%, 160%, 180%, 200% or more, higher or lower than a control level indicates lymphocyte activation. In some embodiments, a level of one or more expressed or secreted cytokines that is at least 1, 2, 3, 4 or 5 standard deviations greater or lower than the mean of a control level indicates lymphocyte activation. In some embodiments, a level of one or more expressed or secreted cytokines that is at least 1, 2, 3, 4 or 5 median absolute deviations (MADs) greater or lower than a median response level to a control indicates lymphocyte activation. In some embodiments, a control is a negative control, for example, a clone expressing Neon Green (NG).

In some embodiments, a level of one or more expressed or secreted cytokines that is within about 20%, 15%, 10%, 5%, or less, of a control level indicates lymphocyte non-responsiveness or non-stimulation. In some embodiments, a level of one or more expressed or secreted cytokines that is less than 1 or 2 standard deviations higher or lower than the mean of a control level indicates lymphocyte non-responsiveness or non-stimulation. In some embodiments, a level of one or more expressed or secreted cytokines that is less than 1 or 2 median absolute deviations (MADs) higher or lower than a median response level to a control indicates lymphocyte non-responsiveness or non-stimulation.

In some embodiments, lymphocyte stimulation, non-stimulation, inhibition and/or suppression, activation, and/or non-responsiveness is determined by molecular profiling of gene expression, e.g., real-time PCR, of one or more cytokines or other immune mediators described herein.

In some embodiments, a subject response profile can include a quantification, identification, and/or representation of a panel of different cytokines or genes encoding different cytokines, (e.g., 1, 2, 3, 4, 5, 6, 7, 8, 9, 10, 12, 14, 16, 18, 20, or more cytokines) and of the total number of tumor antigens (e.g., of all or a portion of different tumor antigens from the library) that stimulate, do not stimulate, inhibit and/or suppress, activate, or have no or minimal effect on production, expression or secretion of each member of the panel of cytokines or genes.

### Methods of Selecting Tumor Antigens; Methods of Inducing or Inhibiting an Immune Response in a Subject

In general, immune responses can be usefully defined in terms of their integrated, functional end-effects. Dhabar et al. (2014) have proposed that immune responses can be categorized as being immunoprotective, immunopathological, and immunoregulatory/inhibitory. While these categories provide useful constructs with which to organize ideas, an overall in vivo immune response is likely to consist of several types of responses with varying amounts of dominance from each category. Immunoprotective or beneficial responses are defined as responses that promote efficient wound healing, eliminate infections and cancer, and mediate vaccine-induced immunological memory. These responses are associated with cytokines and mediators such as IFN-gamma, IL-12, IL-2, granzyme B, CD107, etc. Immunopathological or deleterious responses are defined as those that are directed against self (autoimmune disease like multiple sclerosis, arthritis, and lupus) or innocuous antigens (asthma, allergies) and responses involving chronic, non-resolving inflammation. These responses can also be associated with molecules that are implicated in immunoprotective responses, but also include immune mediators such as TNF-alpha, IL-10, IL-13, IL-17, IL-4, IgE, histamine, etc. Immunoregulatory responses are defined as those that involve immune cells and factors that regulate (mostly down-regulate) the function of other immune cells. Recent studies suggest that there is an arm of the immune system that functions to inhibit immune responses. For example, regulatory CD4⁺CD25+FoxP3⁺ T cells, IL-10, and TGF-beta, among others have been shown to have immunoregulatory/inhibitory functions. The physiological function of these factors is to keep pro-inflammatory, allergic, and autoimmune responses in check, but they may also suppress anti-tumor immunity and be indicative of negative prognosis for cancer. In the context of tumors, the expression of co-stimulatory molecules often decreases, and the expression of co-inhibitory ligands increases. MHC/HLA molecules are often down-regulated on tumor cells, favoring their escape. The tumor micro-environment, including stromal cells, tumor associated immune cells, and other cell types, produce many inhibitory factors, such as, IL-10, TGF-β, and IDO. Inhibitory immune cells, including Tregs, Tr1 cells, immature DCs (iDCs), pDCs, and MDSC can be found in the tumor micro-environment. (Y Li UT GSBS Thesis 2016). Examples of mediators and their immune effects are shown in Table 2.

TABLE 2
Immune Mediators

| | | | Beneficial | | | Deleterious | | |
|---|---|---|---|---|---|---|---|---|
| | | | Outcomes | | | Outcomes | | |
| Cytokine | Function | Secreted by | Cancer | ID | AI | Cancer | ID | AI |
| TRAIL | Induces apoptosis of | Most cells | X | X | ? | X | ? | ? |
| | tumor cells, induces | | | | | | | |
| | immune suppressor | | | | | | | |
| | cells | | | | | | | |
| IFN- | Critical for innate | T cells, | X | X | ? | X | ? | X |
| gamma | and adaptive immunity | NK cells, | | | | | | |
| | to pathogens, inhibits | NKT cells | | | | | | |
| | viral replication, | | | | | | | |
| | increases MHC Class | | | | | | | |
| | I expression | | | | | | | |
| IL-12 | Th1 differentiation; | DCs, macro- | X | X | ? | X | ? | X |
| | stimulates T cell | phages, | | | | | | |
| | growth, induces | neutron- | | | | | | |
| | IFN-gamma/TNF-alpha | phils | | | | | | |
| | secretion from T cells, | | | | | | | |
| | enhances CTLs | | | | | | | |
| IL-2 | T cell proliferation, | T cells, APCs | X | X | X | ? | ? | ? |
| | differentiation into | | | | | | | |
| | effector and memory | | | | | | | |
| | T cells and | | | | | | | |
| | regulatory T cells | | | | | | | |
| TNF- | Induces fevers, | Macro- | X | X | ? | X | ? | X |
| alpha | apoptosis, | phages, | | | | | | |
| | inflammation, | APCs | | | | | | |
| | inhibits viral | | | | | | | |
| | replication | | | | | | | |
| MIP-1 | Chemotactic/pro- | Macro- | X | X | ? | ? | ? | X |
| alpha | inflammatory | phages, DCs, | | | | | | |
| | effects, activates | T cells | | | | | | |
| | granulocytes, | | | | | | | |
| | induces secretion of | | | | | | | |
| | IL-1/IL6/TNF-alpha | | | | | | | |
| MIP-1 | Chemotactic/pro- | Macro- | X | X | ? | ? | ? | X |
| beta | inflammatory | phages, DCs, | | | | | | |
| | effects, activates | T cells | | | | | | |
| | granulocytes, induces | | | | | | | |
| | secretion of | | | | | | | |
| | IL-1/IL6/TNF-alpha | | | | | | | |
| CXCL9 | T cell | APCs | X | X | ? | X | ? | X |
| | chemoattractant, | | | | | | | |
| | induced by IFN-gamma | | | | | | | |
| CXCL10 | Chemoattractant for | APCs | X | X | ? | ? | ? | X |
| | T cells, macrophages, | | | | | | | |
| | NK and DCs, promotes | | | | | | | |
| | T cell adhesion to | | | | | | | |
| | endothelial cells | | | | | | | |
| MCP-1 | Recruits monocytes, | most cells | X | X | ? | X | ? | X |
| | memory T cells and | | | | | | | |
| | DCS | | | | | | | |
| RANTES | Recruits T cells, | T cells | X | X | ? | ? | ? | X |
| | eosinophils, | | | | | | | |
| | basophils, induces | | | | | | | |
| | proliferation/ | | | | | | | |
| | activation of NK | | | | | | | |
| | cells, T cell | | | | | | | |
| | activation marker | | | | | | | |
| CXCL11 | Chemoattractant for | APCs | X | X | ? | ? | ? | X |
| | activated T cells | | | | | | | |
| IL-3 | Stimulates | T cells, APCs | X | X | ? | ? | ? | ? |
| | proliferation of | | | | | | | |
| | myeloid cells, | | | | | | | |
| | induces growth of | | | | | | | |
| | T cells | | | | | | | |
| IL-17 | Produced by Th17 | T cells | X | X | ? | X | ? | X |
| I | cells, induces | | | | | | | |
| | production of IL6, | | | | | | | |
| | GCSF, GMCSF, IL1b, | | | | | | | |
| | TGF-beta, TNF-alpha, | | | | | | | |
| | chemokines | | | | | | | |
| IL-18 | Pro-inflammatory, | Macro- | X | X | ? | X | ? | X |
| | induces cell-mediated | phages | | | | | | |
| | immunity, production | | | | | | | |
| | of IFN-gamma | | | | | | | |
| IL-21 | Induces proliferation, | CD4 T cells | X | X | X | X | ? | ? |
| | upregulated in | | | | | | | |
| | Th2/Th17 TFh | | | | | | | |
| IL-22 | Cell-mediated | NK cells, | X | X | ? | X | ? | X |
| | immunity, pro- | T cells | | | | | | |
| | inflammatory | | | | | | | |
| IL-23 | Pro-inflammatory | APCs | X | X | ? | X | ? | X |
| IL-24 | Controls survival | Monocytes | X | X | ? | ? | ? | X |
| | and proliferation | macro- | | | | | | |
| | | phages, | | | | | | |
| | | Th2 cells | | | | | | |
| IL-27 | Induces differentiation | APCs, T cells | X | X | X | X | ? | X |
| | of T cells, upregulates | | | | | | | |
| | IL-10, can be pro-or | | | | | | | |
| | anti-inflammatory; | | | | | | | |
| | promotes Th1/Tr1, | | | | | | | |
| | inhibits Th2/Th17/ | | | | | | | |
| | regulatory T cells | | | | | | | |
| IL-32 | Pro-inflammatory, | T cells, | X | X | ? | X | ? | X |
| | increases secretion | NK cells | | | | | | |
| | of inflammatory | | | | | | | |
| | cytokines and | | | | | | | |
| | chemokines | | | | | | | |
| CSF | Induces myeloid cells | APCs | X | X | X | ? | ? | ? |
| | to proliferate and | | | | | | | |
| | differentiate | | | | | | | |
| GM-CSF | Promotes macrophage | T cells, | X | X | ? | ? | ? | X |
| | and Eosinophil | macro- | | | | | | |
| | proliferation and | phages | | | | | | |
| | maturation, growth | | | | | | | |
| | factor | | | | | | | |
| TRANCE | Helps DC maturation/ | T cells | ? | X | ? | X | ? | ? |
| | survival, T cell | | | | | | | |
| | activation marker, | | | | | | | |
| | anti-apoptotic, | | | | | | | |
| | stimulates osteoclast | | | | | | | |
| | activity | | | | | | | |
| MIP-3 | Chemotactic for T | | X | X | ? | ? | ? | X |
| alpha | cells, DCs | | | | | | | |
| fractalkine | Chemotactic for T | Endothelial | X | X | ? | ? | ? | X |
| | cells and monocytes | cells | | | | | | |
| IL-4 | Stimulates B cells, | Th2 cells, | ? | X | ? | X | X | X |
| | Th2 proliferation, | basophils | | | | | | |
| | plasma cell | | | | | | | |
| | differentiation, IgE, | | | | | | | |
| | upregulates MHC | | | | | | | |
| | Class II expression, | | | | | | | |
| | decreases IFN- | | | | | | | |
| | gamma production | | | | | | | |
| IL-10 | Downregulates Th1 | Monocytes | X | ? | X | X | X | X |
| | cytokines/MHC Class | Th2 cells, | | | | | | |
| | II expression/Co- | regulatory | | | | | | |
| | stimulatory molecule | T cells | | | | | | |
| | expression | | | | | | | |
| IL-5 | Stimulates B cells, | Th2 cells, | ? | X | ? | X | X | X |
| | Ig secretion, eosinophil | mast cells | | | | | | |
| | activation | | | | | | | |
| IL-13 | Similar to IL4, induces | Th2 cells, | ? | X | ? | X | X | X |
| | IgE production, Th2 | NK cells, | | | | | | |
| | cytokine | mast cells, | | | | | | |
| | | eosinophils, | | | | | | |
| | | basophils | | | | | | |
| TGF-beta | Inhibits T cell | regulatory | ? | ? | X | X | X | ? |
| | proliferation, | T cells | | | | | | |
| | activity, function; | | | | | | | |
| | blocks effects of | | | | | | | |
| | pro-inflammatory | | | | | | | |
| | cytokines | | | | | | | |
| IL-1 beta | Induces fevers, pro- | Macro- | X | X | ? | X | ? | X |
| | inflammatory | phages | | | | | | |
| IL-6 | Pro-inflammatory, | T cells, | ? | X | ? | X | X | X |
| | drives osteoclast | macro- | | | | | | |
| | formation, drives | phages | | | | | | |
| | Th17 | | | | | | | |
| IL-8 | Recruits neutrophils | Macro- | ? | X | ? | X | ? | X |
| | to site of infection | phages, | | | | | | |
| | | epithelial | | | | | | |
| | | cells | | | | | | |
| IL-31 | Cell-mediated immunity, | Th2 cells, | X | X | ? | X | ? | X |
| | pro-inflammatory | macro- | | | | | | |
| | | phages, DCs | | | | | | |
| IL-15 | T cell proliferation | T cells, | X | X | X | ? | ? | ? |
| | and survival | NK cells | | | | | | |
| IL-9 | Th2 proliferation, | T cells, | ? | ? | X | X | X | ? |
| | cytokine secretion | neutrophils, | | | | | | |
| | | mast cells | | | | | | |

ID = Infectious disease
IA = Autoimmune disease

The disclosure provides methods and systems for identifying and selecting (or deselecting) tumor antigens (e.g., stimulatory and/or inhibitory antigens). In some embodiments, a stimulatory antigen is a tumor antigen (e.g., a tumor antigen described herein) that stimulates one or more lymphocyte responses that are beneficial to the subject. In some embodiments, a stimulatory antigen is a tumor antigen (e.g., a tumor antigen described herein) that inhibits and/or suppresses one or more lymphocyte responses that are deleterious or non-beneficial to the subject. Examples of immune responses that may lead to beneficial anti-tumor responses (e.g., that may enhance immune control of a tumor) include but are not limited to 1) cytotoxic CD8⁺ T cells which can effectively kill cancer cells and release the mediators perforin and/or granzymes to drive tumor cell death; and 2) CD4⁺ Th1 T cells which play an important role in host defense and can secrete IL-2, IFN-gamma and TNF-alpha. These are induced by IL-12, IL-2, and IFN-gamma among other cytokines.

In some embodiments, an inhibitory antigen is a tumor antigen (e.g., a tumor antigen described herein) that stimulates one or more lymphocyte responses that are deleterious or non-beneficial to the subject. In some embodiments, an inhibitory antigen is a tumor antigen (e.g., a tumor antigen described herein) that inhibits and/or suppresses one or more lymphocyte responses that are beneficial to the subject. Examples of immune responses that may lead to deleterious or non-beneficial anti-tumor responses (e.g., that may impair or reduce control of a tumor) include but are not limited to 1) T regulatory cells which are a population of T cells that can suppress an immune response and secrete immunosuppressive cytokines such as TGF-beta and IL-10 and express the molecules CD25 and FoxP3; and 2) Th2 cells which target responses against allergens but are not productive against cancer. These are induced by increased IL-4 and IL-10 and can secrete IL-4, IL-5, IL-6, IL-9 and IL-13.

Additionally or alternatively, tumor antigens may be identified and/or selected (or de-selected) based on association with desirable or beneficial responses, e.g., clinical responses. Additionally or alternatively, tumor antigens may be identified and/or selected (or de-selected) based on association with undesirable, deleterious or non-beneficial responses, e.g., clinical responses. Tumor antigens may be identified and/or selected (or de-selected) based on a combination of the preceding methods, applied in any order.

Responses whereby tumor antigens or immunogenic fragments thereof (i) stimulate lymphocyte responses that are beneficial to the subject, (ii) stimulate expression of cytokines that are beneficial to the subject, (iii) inhibit and/or suppress lymphocyte responses that are deleterious or non-beneficial to the subject, or (iv) inhibit and/or suppress expression of cytokines that are deleterious or non-beneficial to the subject, are termed “beneficial responses”.

In some embodiments, a selected tumor antigen stimulates one or more lymphocyte responses that are beneficial to the subject. In some embodiments, a selected tumor antigen inhibits and/or suppresses one or more lymphocyte responses that are deleterious or non-beneficial to the subject.

In some embodiments, a selected tumor antigen increases expression and/or secretion of cytokines that are beneficial to the subject. In some embodiments, a selected tumor antigen inhibits and/or suppresses expression of cytokines that are deleterious or non-beneficial to the subject.

In some embodiments, administration of one or more selected tumor antigens to the subject elicits an immune response of the subject. In some embodiments, administration of one or more selected tumor antigens to the subject elicits a beneficial immune response of the subject. In some embodiments, administration of one or more selected tumor antigens to the subject elicits a beneficial response of the subject. In some embodiments, administration of one or more selected tumor antigens to the subject improves clinical response of the subject to a cancer therapy.

Responses whereby tumor antigens or immunogenic fragments thereof (i) stimulate lymphocyte responses that are deleterious or not beneficial to the subject, (ii) stimulate expression of cytokines that are deleterious or not beneficial to the subject, (iii) inhibit and/or suppress lymphocyte responses that are beneficial to the subject, or (iv) inhibit and/or suppress expression of cytokines that are beneficial to the subject, are termed “deleterious or non-beneficial responses”.

In some embodiments, one or more tumor antigens are selected (or de-selected) based on association with desirable or beneficial immune responses. In some embodiments, one or more tumor antigens are selected (or de-selected) based on association with undesirable, deleterious, or non-beneficial immune responses.

In some embodiments, a selected tumor antigen stimulates one or more lymphocyte responses that are deleterious or non-beneficial to the subject. In some embodiments, a selected tumor antigen inhibits and/or suppresses one or more lymphocyte responses that are beneficial to the subject.

In some embodiments, a selected tumor antigen increases expression and/or secretion of cytokines that are deleterious or non-beneficial to the subject. In some embodiments, a selected tumor antigen inhibits and/or suppresses expression of cytokines that are beneficial to the subject.

In some embodiments, the one or more tumor antigens are de-selected by the methods herein.

In some embodiments, the one or more selected tumor antigens are excluded from administration to a subject.

### Methods of Selecting Potential Tumor Antigens

In well-established tumors, activation of endogenous anti-tumor T cell responses is often insufficient to result in complete tumor regression. Moreover, T cells that have been educated in the context of the tumor micro-environment sometimes are sub-optimally activated, have low avidity, and ultimately fail to recognize the tumor cells that express antigen. In addition, tumors are complex and comprise numerous cell types with varying degrees of expression of mutated genes, making it difficult to generate polyclonal T cell responses that are adequate to control tumor growth. As a result, researchers in the field have proposed that it is important in cancer subjects to identify the mutations that are “potential tumor antigens” in addition to those that are confirmed in the cancer subject to be recognized by their T cells.

There are currently no reliable methods of identifying potential tumor antigens in a comprehensive way. Computational methods have been developed in an attempt to predict what is an antigen, however there are many limitations to these approaches. First, modeling epitope prediction and presentation needs to take into account the greater than 12,000 HLA alleles encoding MHC molecules, with each subject expressing as many as 14 of them, all with different epitope affinities. Second, the vast majority of predicted epitopes fail to be found presented by tumors when they are evaluated using mass spectrometry. Third, the predictive algorithms do not take into account T cell recognition of the antigen, and the majority of predicted epitopes are incapable of eliciting T cell responses even when they are present. Finally, the second subset of T cells, the CD4+ T cell subset, is often overlooked; the majority of in silico tools focus on MHC/HLA class I binders. The tools for predicting MHC/HLA class II epitopes are under-developed and more variable.

The present disclosure provides methods to a) identify polypeptides that are potential tumor antigens in antigen presentation assays of the disclosure, and b) select polypeptides on the basis of their antigenic potential. The methods are performed without making predictions about what could be a target of T cell responses or presented by MHC/HLA, and without the need for deconvolution. The methods can be expanded to explore antigenic potential in healthy subjects who share the same HLA alleles as a subject, to identify those potential tumor antigens that would be most suitable to include in an immunogenic composition or vaccine formulation. The methods ensure that the potential tumor antigen is processed and presented in the context of subject HLA molecules, and that T cells can respond to the potential tumor antigen if they are exposed to the potential tumor antigen under the right conditions (e.g., in the context of a vaccine with a strong danger signal from an adjuvant or delivery system).

The preceding methods for selection of tumor antigens may be applied to selection of potential tumor antigens, e.g., polypeptides encoding one or more mutations present or expressed in a cancer or tumor cell of a subject, and any other tumor antigens described herein.

### Methods of Making T Cell Compositions for Autologous Adoptive Cell Therapy

### Overall Rationale

Certain methods of the disclosure are directed to stimulating and expanding antigen-specific T cells of a cancer patient to make a highly effective, personalized or non-personalized, autologous adoptive T cell therapy. The autologous adoptive T cell therapy increases the likelihood of tumor eradication and has the potential to limit metastatic tumor escape. Identification and selection of tumor-specific antigens to stimulate and expand the cancer patient's T cells ex vivo is achieved using ATLAS, an immune response profiling method that enables comprehensive screening of a tumor mutanome. The tumor-specific antigens used to stimulate and expand the T cells ex vivo may be patient-specific (personal), or may be shared by a cohort of patients, or may comprise both patient-specific (personal) and shared antigens.

Nearly two decades of experience exploring various autologous antigen-specific adoptive cell therapies in clinical trials (tumor infiltrating lymphocytes [TILs], tumor-associated antigen-specific T cells [TAA-specific T cells], bispecific antibody-[BiAb-] activated T cells [ATC], etc.), support their safety and efficacy. Advantages provided by autologous adoptive cell therapy methods of the disclosure over these comparable adoptive cell therapies include: i) the ATLAS method permits selection of tumor antigens that elicit robust T cell responses and are not expressed on normal cells, thus addressing tumor heterogeneity and potentially reducing toxicities associated with targeting normal tissues; ii) the ATLAS method has revealed inhibitory, potentially tumor-promoting responses to antigens that will be avoided in autologous adoptive cell therapy methods of the disclosure; and iii) isolated T cells reactive for patient- or patient cohort-specific antigens are selected for ex vivo T cell expansion, thus enriching autologous adoptive cell therapy methods of the disclosure for T cells that are specific for the patient's tumor.

### Tumor Antigens as Targets for Adoptive Cellular Therapy

During the process of oncogenesis, cancers acquire thousands of diverse somatic mutations, some of which interfere with cell regulation and help to drive cell proliferation and resistance to cancer treatments. These mutations often alter amino acid coding sequences or intron splicing, causing tumors to express mutant proteins that are not expressed by healthy, normal cells. In some cases, cancers arise from oncovirus-driven mutations. In humans, abnormal (mutant or foreign) protein sequences are processed into short peptides and presented on the cell surface in the context of human leukocyte antigen (HLA) for recognition by T cells as foreign antigens. Tumor-specific antigens resulting from genetic mutations, or alternatively from post-translational peptide fusion events, are called neoantigens. Indeed, early studies showed that patient T cells are reactive against specific neoantigens from a patient's tumor, and in the case of oncovirus-driven cancers, against viral antigens as well. Additionally, when a patient's tumor-infiltrating lymphocytes (TILs), which were reactive to mutated forms of at least two neoantigens from a patient's melanoma tumor, were expanded ex vivo and adoptively transferred back to the patient, complete tumor regression was observed. The neoantigen-specific T cells were found at high levels in the tumor up to 1 month after transfer. Although such findings support the importance of neoantigens in the naturally occurring anti-tumor T cell response, not all somatic mutations will result in high immunogenicity, specificity, or expression on a patient's tumor cells. Accordingly, the methods of the disclosure provide the benefit of pre-screening a patient's or a patient cohort's T cell responses to their identified somatic mutations in order to select stimulatory tumor antigens, including but not limited to neoantigens, that have induced putatively beneficial T cell responses, which can be subsequently optimized and amplified for protective anti-tumor immune responses.

### Limitations of Existing Antigen Selection Technologies

Current methods applied across academic and industry clinical programs to select antigens for personalized drug development rely largely on the use of software algorithms. The most broadly applied software tools attempt to identify neoepitopes through prediction of MHC/HLA binding affinity for short peptide sequences. Other tools are available that attempt to predict how mutant proteins will be processed into shorter peptides through proteasomal cleavage. In addition, there are tools to predict which peptides will be transported effectively into the endoplasmic reticulum and are therefore more likely to be effectively loaded into HLA molecules for presentation to T cells.

Currently, however, there are numerous challenges with each of these software tools, with the accuracy of these predictions relying on the quality of the biological data available to train the models. For example, the diversity of HLA class I molecules across individuals is such that it is impossible to accurately predict binding affinities for every allele. In addition, HLA class II antigen presentation is more promiscuous, and algorithms have fallen short in their ability to predict epitopes for recognition by CD4⁺ T cells. Finally, peptide binding into MHC/HLA is only one aspect of antigenicity, and the ability to identify T cells that recognize a given antigen is missing from current peptide algorithm or peptide elution approaches.

### Rationale for ATLAS as the Antigen Identification and Selection Method

The ATLAS method, as further described in WO 2018/175505, allows rapid, high-throughput identification of pre-existing, antigen-specific T cell responses without the use of in silico down-selection criteria. ATLAS eliminates many of the aforementioned challenges associated with the use of prediction tools for tumor antigen selection by providing the following advantages: i) it empirically identifies tumor antigens using the patient's own T cells and professional and/or non-professional antigen presenting cells, e.g., monocyte-derived dendritic cells (MDDCs), instead of computer-based predictions that require validation; ii) it comprehensively covers each patient's own HLA specificities; iii) it separately identifies tumor antigens for both CD4⁺ and CD8⁺ T cell subsets; and iv) it facilitates tumor antigen selection based on biologically relevant T cell responses.

To date, the ATLAS method has been used to profile T cell responses for multiple proteomic libraries, ranging from a few dozen to over 2,000 expressed genes from HSV-2, Streptococcus pneumoniae, Chlamydia trachomatis, Plasmodium falciparum, human papilloma virus, and Epstein-Barr virus. In oncology, ATLAS has also been used to screen putative neoantigens, oncoviral antigens, melanoma tumor-associated antigens, colorectal cancer-associated antigens, and lung tumor-associated antigens. In all cases, ATLAS has enabled comprehensive screening of potential tumor antigens using autologous cells and identified targets of pre-existing stimulatory as well as potentially unwanted (i.e., inhibitory) antigen-specific T cell responses. In the personalized setting, the method provides greater confidence that each patient has generated T cell responses to the selected therapeutic tumor antigen target, and that those tumor antigens have been expressed in the patient's tumor. Applicant seeks to augment these responses with an autologous adoptive cell therapy (GEN-011). In further support of this rationale, preliminary findings from a Phase 1/2a clinical trial of a targeted personalized cancer vaccine (GEN-009) showed that ATLAS screening successfully yielded stimulatory antigens, which when administered as a vaccine formulation, resulted in increased immune responses to the majority of those tumor antigens. ATLAS screening at the same time allowed exclusion of inhibitory antigens. Accordingly, in some embodiments, the ATLAS method for patient-specific or patient cohort-specific T cell antigen identification and selection provides the means to prioritize stimulatory antigens and corresponding peptide pools used to stimulate and expand the patient's autologous T cells.

### Summary of Methods of Making T Cell Compositions for Autologous Adoptive Cell Therapy

In some embodiments, the present disclosure provides methods for making an antigen-specific autologous adoptive cell therapy. In some embodiments, the autologous adoptive cell therapy is useful for treatment of patients with solid or liquid tumors. In some embodiments, the solid tumors include, but are not limited to, melanoma, malignant melanoma (MM), Merkel cell carcinoma (MCC), cutaneous squamous cell carcinoma (CSCC), non-small cell lung cancer (NSCLC), small cell lung cancer (SCLC), large cell lung cancer (LCLC), tracheobronchial cancer, pleomorphic carcinoma, squamous cell lung carcinoma (SqCLC), squamous cell carcinoma of the head and neck (SCCHN), nasopharyngeal carcinoma (NPC), urothelial carcinoma (bladder, ureter, urethra, or renal pelvis), renal cell carcinoma (RCC), or anal squamous cell carcinoma (ASCC). In some embodiments, the solid tumors include, but are not limited to, breast cancer, endometrial cancer, cervical cancer, ovarian cancer, pancreatic cancer, colorectal cancer, prostate cancer, chondrosarcoma, osteosarcoma, or thyroid cancer. In some embodiments, the autologous adoptive cell therapy is custom-manufactured for each individual cancer patient.

An exemplary workflow for making an antigen-specific autologous adoptive cell therapy of the disclosure is shown in FIG. 5 and further described in Example 4. Briefly, next-generation sequencing (NGS) is used to identify genomic variants or fusion events (or viral sequences) within a patient's tumor that are specific to the tumor cells and not found within the patient's germline DNA and/or RNA sequences. These mutations are subsequently screened using Applicant's ATLAS method for rapid, high-throughput identification and selection of antigens that elicit specific T cell responses. Overlapping peptides corresponding to the ATLAS-selected antigens, e.g. stimulatory antigens, are then synthesized and pooled (OLP pool). CD14⁺ monocytes and T cells are sorted from peripheral blood of a patient. The CD14⁺ monocytes are differentiated and matured into monocyte-derived dendritic cells (MDDCs). The MDDCs, OLP pool, and T cells are cultured together. After multiple days of antigen-specific stimulation and expansion, the cells are re-stimulated. Antigen-specific T cells are isolated based on upregulation of one or both of the T cell activation markers, CD137 and CD154. The enriched antigen-specific T cells are then rapidly and non-specifically expanded with anti-CD3/CD28 antibodies.

### Methods of Obtaining T Cells

In certain embodiments of the disclosure, a source of T cells can first be obtained, e.g., from a subject. Non-limiting examples of subjects include humans, dogs, cats, mice, rats, and transgenic species thereof. As described herein, T cells or PBMCs enriched for or depleted of a certain population of T cells can be administered to a subject. Thus, the T cells will have an immunocompatibility relationship to a recipient subject, and any such relationship is contemplated for use according to the present disclosure.

For example, the T cells can be syngeneic to a recipient subject. The term “syngeneic” refers to the state of deriving from, originating in, or being members of the same species that are genetically identical, particularly with respect to antigens or immunological reactions. These include identical twins having matching MHC/HLA types.

T cells can be “autologous” if the transferred cells are obtained from and transplanted to the same subject.

T cells can be “matched allogeneic” if the transferred cells are obtained from and transplanted to different members of the same species, yet have sufficiently matched major histocompatibility complex (MHC/HLA) antigens to avoid an adverse immunogenic response. Determining the degree of MHC/HLA mismatch may be accomplished according to standard tests known and used in the art (see, e.g., Mickelson and Petersdorf (1999) Hematopoietic Cell Transplantation, Thomas, E. D. et al. eds., pg 28-37, Blackwell Scientific, Malden, Mass.; Vaughn, Method. Mol. Biol. MHC Protocol. 210:45-60 (2002); Morishima et al., Blood 99:4200-4206 (2002)).

T cells can be “mismatched allogeneic”, which refers to deriving from, originating in, or being members of the same species having non-identical MHC/HLA antigens (i.e., proteins) as typically determined by standard assays used in the art, such as serological or molecular analysis of a defined number of MHC/HLA antigens, sufficient to elicit adverse immunogenic responses. A “partial mismatch” refers to partial match of the MHC/HLA antigens tested between members, typically between a donor and recipient. For instance, a “half mismatch” (haplo-mismatch) refers to 50% of the MHC/HLA antigens tested as showing different MHC/HLA antigen type between two members. A “full” or “complete” mismatch refers to all MHC/HLA antigens tested as being different between two members.

T cells can be “xenogeneic”, which refers to deriving from, originating in, or being members of different species, e.g., human and rodent, human and swine, human and chimpanzee, etc. Further, T cells can be “transgenic”, e.g., engineered to express a T cell receptor specific for a stimulatory antigen, or to relieve checkpoint inhibition.

T cells can be obtained from a number of sources, including peripheral blood mononuclear cells (PBMCs), bone marrow, lymph node tissue, spleen tissue, thymic tissue, tumor issue, and umbilical cord. In certain embodiments, any number of T cell lines available in the art, may be used. In certain embodiments, T cells are obtained from a unit of blood collected from a subject using any number of techniques known to the skilled artisan, such as Ficoll separation. For example, cells from the circulating blood of a subject can be obtained by apheresis or leukapheresis. The apheresis product typically contains lymphocytes, including T cells, monocytes, granulocytes, B cells, other nucleated white blood cells, red blood cells, and platelets. In some embodiments, the cells collected by apheresis can be washed to remove the plasma fraction and to place the cells in an appropriate buffer or medium for subsequent processing steps.

In another method, T cells are isolated from peripheral blood by lysing red blood cells and depleting monocytes, for example, by centrifugation through a PERCOLL™ gradient or adherence to plastic. Alternatively, T cells can be isolated from blood harvested from umbilical cord. Alternatively, T cells can be isolated from tumor tissue by enzymatic digestion and/or mechanical disruption.

A plurality of T cells of interest (e.g., T cells that mediate an immune response to a stimulatory antigen that enhances immune control of a tumor or cancer) can then be obtained or isolated (e.g., sorted) from an initial source, e.g., a sample of PBMCs. In one embodiment, fluorescence activated cell sorting (FACS) or magnetic activated cell sorting (MACS), is used to sort, analyze, and/or isolate T cells of interest. For example, cells having a cellular marker or other specific marker of interest can be tagged with an antibody, or a mixture of antibodies, that bind one or more of the cellular markers. Each antibody directed to a different marker can be conjugated to a detectable molecule, e.g., a fluorescent dye that may be distinguished from other fluorescent dyes coupled to other antibodies. A stream of tagged or “stained” cells can be passed through a light source that excites the fluorochrome and the emission spectrum from the cells detected to determine the presence of a particular labeled antibody. By concurrent detection of different fluorochromes (multicolor fluorescence cell sorting), cells displaying different sets of cell markers can be identified and isolated from other cells in the population. Other FACS and MACs parameters, including, e.g., side scatter (SSC), forward scatter (FSC), and vital dye staining (e.g., with propidium iodide) allow selection of cells based on size and viability. FACS and MACS sorting and analysis are well-known in the art and described in, for example, U.S. Pat. Nos. 5,137,809; 5,750,397; 5,840,580; 6,465,249; Miltenyi, et al., Cytometry 11:231-238 (1990). General guidance on fluorescence activated cell sorting is described in, for example, Shapiro (2003) Practical Flow Cytometry, 4th Ed., Wiley-Liss (2003) and Ormerod (2000) Flow Cytometry: A Practical Approach, 3rd Ed., Oxford University Press.

Another method of isolating T cells of interest involves a solid or insoluble substrate to which is bound antibodies or ligands that interact with specific cell surface markers. In immunoadsorption techniques, cells can be contacted with the substrate (e.g., column of beads, flasks, magnetic particles, etc.) containing the antibodies and any unbound cells removed. Immunoadsorption techniques can be scaled up to deal directly with the large numbers of cells in a clinical harvest. Suitable substrates include, e.g., plastic, cellulose, dextran, polyacrylamide, agarose, and others known in the art (e.g., Pharmacia Sepharose 6 MB macrobeads). When a solid substrate comprising magnetic or paramagnetic beads is used, cells bound to the beads can be readily isolated by a magnetic separator (see, e.g., Kato et al., Cytometry 14:384-92 (1993)). Affinity chromatographic cell separations can involve passing a suspension of cells over a support bearing a selective ligand immobilized to its surface. The ligand interacts with its specific target molecule on the cell and is captured on the matrix. The bound cell is released by the addition of an elution agent to the running buffer of the column and the free cell is washed through the column and harvested as a homogeneous population. As apparent to the skilled artisan, adsorption techniques may use nonspecific adsorption.

FACS, MACS, and most batch-wise immunoadsorption techniques can be adapted to both positive and negative selection procedures (see, e.g., U.S. Pat. No. 5,877,299). In positive selection, the desired cells are labeled with antibodies and removed away from the remaining unlabeled/unwanted cells. In negative selection, the unwanted cells are labeled and removed. Another type of negative selection that may be employed is use of antibody/complement treatment or immunotoxins to remove unwanted cells.

In some embodiments, a population of cells can be obtained (e.g., using a sorting method described herein) and used in methods of the disclosure that comprises more than about 40%, 45%, 50%, 55%, 60%, 65%, 70%, 75%, 80%, 85%, 90%, 95%, 96%, 97%, 98%, 99%, or more (e.g., about 65% to about 90%, about 65% to about 95%, about 80% to about 90%, about 80% to about 95%, about 85% to about 90%, about 85% to about 95%, or about 90% to about 95%), cells of interest (e.g., T cells that mediate an immune response to at least one stimulatory antigen). In some embodiments, a population of cells (e.g., a depleted cell population described herein) can be obtained (e.g., using a sorting method described herein) and used in methods of the disclosure that comprises less than about 40%, 35%, 30%, 25%, 20%, 15%, 10%, 5%, 4%, 3%, 2%, 1%, or less (e.g., about 5% to about 10%, about 4% to about 10%, about 3% to about 10%, about 2% to about 10%, about 1% to about 10%, about 1% to about 5%, or about 2% to about 5%), or lack any detectable, cells of interest (e.g., T cells that mediate an immune response to at least one stimulatory antigen).

The obtained populations of cells can be used directly in a method of the disclosure, or can be frozen for use at a later date using a known method. For example, cells can be frozen using a freezing medium comprising 5-10% DMSO, 10-90% serum albumin, and 50-90% culture medium, or using a commercially available medium such as CS10 (STEMCELL Technologies). Other additives useful for preserving cells include, e.g., disaccharides such as trehalose (Scheinkonig et al., Bone Marrow Transplant. 34:531-536 (2004)), a plasma volume expander (such as hetastarch), and/or isotonic buffer solutions (such as phosphate-buffered saline). Compositions and methods for cryopreservation are well-known in the art (see, e.g., Broxmeyer et al., Proc. Natl. Acad. Sci. U.S.A. 100:645-650 (2003)).

Methods of Stimulating and/or Expanding Antigen-Specific T Cells

In some embodiments, methods include culturing T cells with an effective amount of an agent or a combination of agents for a certain period of time in order to stimulate and/or expand the T cells. In some embodiments the T cells may be cultured with an effective amount of an agent or combination of agents for e.g., at least 6, 12, 18, 24, 30, 36, 42, 48, or more hours. In some embodiments, the T cells may be cultured with an effective amount of an agent or combination of agents for e.g., at least 1, 2, 3, 4, 5, 6, 7, 8, 9, 10, 14, 21 or more days. In some embodiments, the expansion step is performed for no more than 5, 4, 3, 2, or 1 day.

Once the T cells are stimulated and/or expanded, they can then be re-administered to the subject. For example, a cellular therapeutic comprising the stimulated and/or expanded T cells can be administered to the subject. To determine that the T cell populations are stimulated and/or expanded, T cells may be assayed using antigen presentation assays and/or assayed for certain cell markers expressed on the T cells as previously described.

In another embodiment, T cells that are responsive to a stimulatory antigen may be isolated from PBMCs from a subject. T cells responsive to a stimulatory antigen may be isolated from the PBMCs using a particular combination of reagents and culture medium in the presence of the stimulatory antigen. For example, tetramers, bi-specific cytokine capture reagents, and antibodies could be used. The isolated T cells may be further stimulated and/or expanded using an effective amount of an agent or a combination of agents. In another embodiment, stimulated and/or expanded T cells may be pooled with PBMCs from which they were isolated from and/or may be pooled with additional unexpanded or expanded T cells prior to administration to the subject. In some embodiments, the T cells may be expanded ex vivo and then administered to the subject. In some embodiments, the T cells may be concurrently stimulated and expanded ex vivo, then administered to the subject.

In other embodiments, PBMCs are obtained from a cancer patient and the T cells present in the PBMCs that are responsive to an inhibitory antigen are identified. The T cells identified may then be depleted ex vivo. T cells in the remaining fraction of PBMCs (i.e., depleted of T cells responsive to an inhibitory antigen) may be stimulated with one or more stimulatory antigens and may optionally be expanded non-specifically. PBMCs including the stimulated T cells may then be administered back to the cancer patient.

In some embodiments, autologous or HLA matched allogenenic PBMCs are stimulated with one or more stimulatory antigens and/or expanded, and such PBMCs are administered to the subject in order to induce one or more beneficial immune responses. In some embodiments, a T cell receptor from T cells specific for stimulatory antigens are isolated and transduced into new T cells from the same subject or an HLA-matched allogeneic individual to elicit a beneficial response.

### Production of Tumor Antigens

A tumor antigen (e.g., a tumor antigen described herein) or peptides spanning a tumor antigen suitable for use in any method or composition of the disclosure may be produced by any available means, such as recombinantly or synthetically (see, e.g., Jaradat Amino Acids 50:39-68 (2018); Behrendt et al., J. Pept. Sci. 22:4-27 (2016)). For example, a tumor antigen or peptides spanning a tumor antigen may be recombinantly produced by utilizing a host cell system engineered to express a tumor antigen- or peptide-encoding nucleic acid. Alternatively or additionally, a tumor antigen may be produced by activating endogenous genes. Alternatively or additionally, a tumor antigen or peptides spanning a tumor antigen may be partially or fully prepared by chemical synthesis. Alternatively or additionally, a tumor antigen or peptides spanning a tumor antigen may be produced by coupled in vitro transcription and translation. Alternatively or additionally, a tumor antigen or peptides spanning a tumor antigen may be delivered as one or more plasmids or other form of nucleic acids.

Where proteins or peptides are recombinantly produced, any expression system can be used. To give but a few examples, known expression systems include, for example, E. coli, egg, baculovirus, plant, yeast, or mammalian cells.

In some embodiments, recombinant tumor antigen or peptides suitable for the present invention are produced in mammalian cells. Non-limiting examples of mammalian cells that may be used in accordance with the present invention include BALB/c mouse myeloma line (NSO/l, ECACC No: 85110503); human retinoblasts (PER.C6, CruCell, Leiden, The Netherlands); monkey kidney CV1 line transformed by SV40 (COS-7, ATCC CRL 1651); human embryonic kidney line (HEK293 or 293 cells subcloned for growth in suspension culture, Graham et al., J. Gen Virol., 36:59, 1977); human fibrosarcoma cell line (e.g., HT1080); baby hamster kidney cells (BHK21, ATCC CCL 10); Chinese hamster ovary cells+/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

In some embodiments, the present invention provides recombinant tumor antigen or peptides produced from human cells. In some embodiments, the present invention provides recombinant tumor antigen or peptides produced from CHO cells or HT1080 cells.

Typically, cells that are engineered to express a recombinant tumor antigen or peptides may comprise a transgene that encodes a recombinant tumor antigen or peptides described herein. It should be appreciated that the nucleic acids encoding recombinant tumor antigen or peptides may contain regulatory sequences, gene control sequences, promoters, non-coding sequences and/or other appropriate sequences for expressing the recombinant tumor antigen. Typically, the coding region is operably linked with one or more of these nucleic acid components.

The coding region of a transgene may include one or more silent mutations to optimize codon usage for a particular cell type. For example, the codons of a tumor antigen transgene may be optimized for expression in a vertebrate cell. In some embodiments, the codons of a tumor antigen transgene may be optimized for expression in a mammalian cell. In some embodiments, the codons of a tumor antigen transgene may be optimized for expression in a human cell.

Once a recombinant cell line has been produced, a tumor antigen or polypeptides described herein may be isolated from it. The isolation may be accomplished, for example, by affinity purification techniques or by physical separation techniques (e.g., a size column).

Alternatively or additionally, a tumor antigen or polypeptides described herein may be partially or fully prepared by chemical synthesis. These methods may include chemical synthesis such as solid phase and/or solution phase polypeptide synthesis. See for example, the methodology as described in Bruckdorfer, T. et al. (Curr. Pharm. Biotechnol. 5, 29-43 (2004)).

### Cytokines

In some embodiments, an agent used for activating and/or expanding a lymphocyte may be a cytokine, or a cocktail comprising two or more cytokines. In some embodiments, activation and/or expansion drives a lymphocyte towards a Th1 phenotype (e.g., increases the number and/or proportion of Th1 cells, e.g., cells expressing one or more Th1-associated cytokine, relative to a control). In some embodiments, the agent used for activating and/or expanding a lymphocyte may be a Th1-associated cytokine, or a cocktail comprising two or more Th1-associated cytokines (e.g., IL-2, IL-7, IL-12, IL-15, IL-21, IL-12p40, IFN-gamma). In some embodiments, activation and/or expansion drives a lymphocyte towards a Th2 phenotype (e.g., increases the number and/or proportion of Th2 cells, e.g., cells expressing one or more Th2-associated cytokine, relative to a control). In some embodiments, the agent used for activating and/or expanding a lymphocyte may be a Th2-associated cytokine, or a cocktail comprising two or more Th2-associated cytokines (e.g., IL-4, IL-5, IL-13). In some embodiments, activation and/or expansion drives a lymphocyte towards a Th17 phenotype (e.g., increases the number and/or proportion of Th17 cells, e.g., cells expressing one or more Th17-associated cytokine, relative to a control). In some embodiments, the agent used for activating and/or expanding a lymphocyte may be a Th17-associated cytokine, or a cocktail comprising two or more Th17-associated cytokines (e.g., TGFβ, IL-6, IL-1β, IL-21, IL-23). In some embodiments, activation and/or expansion drive a T cell towards a Tc1, Tc2, or Tc17 phenotype (e.g., increases the number and/or proportion of Tc1, Tc2, or Tc17 cells, e.g., cells expressing one or more Tc 1-, Tc2-, or Tc17-associated cytokines, relative to a control). In some embodiments, the agent used for activating and/or expanding a lymphocyte may be a Tc1, Tc2, or Tc17-associated cytokine, or a cocktail comprising two or more Tc1-, Tc2-, or Tc17-associated cytokines (e.g., IL-12, IL-2; IL-4; TGFβ, IL-6, IL-21).

In some embodiments, an agent used for concurrently activating and expanding a lymphocyte may be a cytokine, or a cocktail comprising two or more cytokines. In some embodiments, concurrent activation and expansion drives a lymphocyte towards a Th1 phenotype (e.g., increases the number and/or proportion of Th1 cells, e.g., cells expressing one or more Th1-associated cytokine, relative to a control). In some embodiments, the agent used for concurrently activating and expanding a lymphocyte may be a Th1-associated cytokine, or a cocktail comprising two or more Th-1 cytokines (e.g., IL-2, IL-7, IL-12, IL-15, IL-21, IL-12p40, IFN-gamma). In some embodiments, concurrent activation and expansion drives a lymphocyte towards a Th2 phenotype (e.g., increases the number and/or proportion of Th2 cells, e.g., cells expressing one or more Th2-associated cytokine, relative to a control). In some embodiments, the agent used for concurrently activating and expanding a lymphocyte may be a Th2-associated cytokine, or a cocktail comprising two or more Th2-associated cytokines (e.g., IL-4, IL-5, IL-13). In some embodiments, concurrent activation and expansion drives a lymphocyte towards a Th17 phenotype (e.g., increases the number and/or proportion of Th17 cells, e.g., cells expressing one or more Th17-associated cytokine, relative to a control). In some embodiments, the agent used for concurrently activating and expanding a lymphocyte may be a Th17-associated cytokine, or a cocktail comprising two or more Th17-associated cytokines (e.g., TGFβ, IL-6, IL-1β, IL-21, IL-23). In some embodiments, concurrent activation and expansion drive a T cell towards a Tc1, Tc2, or Tc17 phenotype (e.g., increases the number and/or proportion of Tc1, Tc2, or Tc17 cells, e.g., cells expressing one or more Tc1-, Tc2-, or Tc17-associated cytokines, relative to a control). In some embodiments, the agent used for activating and/or expanding a lymphocyte may be a Tc1, Tc2, or Tc17-associated cytokine, or a cocktail comprising two or more Tc1-, Tc2-, or Tc17-associated cytokines (e.g., IL-12, IL-2; IL-4; TGF-beta, IL-6, IL-21).

### Chemotherapeutic Agents

In some embodiments, an agent used for activating and/or expanding a lymphocyte may include a chemotherapeutic agent. A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/anti-tumor antibiotics, topoisomerase inhibitors, antibodies, photosensitizers, and kinase inhibitors. Non-limiting examples of chemotherapeutic agents include erlotinib (TARCEVA®, Genentech/OSI Pharm.), docetaxel (TAXOTER®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine,dichloroplatinum(II), CAS No. 15663-27-1), carboplatin (CAS No. 41575-94-4), paclitaxel (TAXOL®, Bristol-Myers Squibb Oncology, Princeton, N.J.), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), and doxorubicin (ADRIAMYCIN®), Akti-1/2, HPPD, and rapamycin.

Additional examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gammalI, calicheamicin omegaI1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside (Ara-C); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

### Methods of Non-Specifically Stimulating T Cells

Methods of the disclosure can include a step of non-specifically activating a population of cells (e.g., an obtained population of T cells described herein). For example, a population of T cells can be non-specifically activated by contacting with an activation agent. Agents that non-specifically activate T cells are known in the art, and any of such agents can be used in a non-specific activation step. Exemplary, non-limiting activating agents include an anti-CD3 antibody, anti-Tac antibody, anti-CD28 antibody, anti-CD2 antibody, and/or phytohemagglutinin (PHA). In some embodiments, a population of T cells is activated by contacting with an anti-CD3 antibody and with an anti-CD28 antibody. For example, a population of T cells can be contacted with beads that include anti-CD3 antibody and anti-CD28 antibody. Such beads are known in the art and commercially available from, e.g., ThermoFisher Scientific.

The non-specific activation step can be performed for, e.g., at least 2, 4, 6, 8, 10, 12, 14, 16, 18, 20, 22, 24, 28, 32, 36, 40, 48, or more hours, at least 1, 2, 3, 4, 5, 6, 7, 8, 9, 10, 12 or more days, or at least 1, 2, 3, 4, or more weeks.

### Methods of Expanding T Cells

Methods of the disclosure can include a step of expanding a population of T cells (e.g., an obtained population of T cells described herein). For example, before or after an activation step described herein, a population of T cells can be expanded by culturing in a suitable cell culture medium that lacks an activation agent. Alternatively, a population of T cells can be activated and expanded concurrently (i.e., in the presence of one or more activation agents described herein). Additionally or alternatively, the expansion step can include culturing a population of T cells in a culture medium comprising, but not limited to, IL-2, IL-7, IL-15, IL-21, IL-12p40, and/or IFN-gamma. In some embodiments, the expansion step can include culturing a population of T cells comprising combinations of two or more of such cytokines.

In some embodiments, T cells are expanded in an antigen-specific manner (e.g., by contacting T cells with one or more specific antigen and with one or more other mediators (not including anti-CD3). In some cases, multiple antigens are combined. In some embodiments, T cells are expanded in a non-specific manner (e.g., not in the presence of an antigen).

The expansion step can be performed, e.g., for at least 6, 12, 18, 24, 30, 36, 42, 48, or more hours, or 1, 2, 3, 4, or more weeks. In some embodiments, the expansion step is performed for at least 1, 2, 3, 4, 5, 6, 7, 8, 9, 10, 11, 12, 14, 18, 21 or more days. In some embodiments, the expansion step is performed for no more than 5, 4, 3, 2, or 1 day.

The expansion step can be performed until the number of cells in the population reaches at least about 10⁴, 10⁵, 10⁶, 10⁷, 10⁸, 10⁹, 10¹⁰, or more cells.

### General Cell Culture Methods

Sorted T cells can be cultured under conditions generally appropriate for T cell culture. Conditions can include an appropriate culture medium that can contain factors for proliferation and viability, including serum (e.g., fetal bovine or human serum), interleukin-2 (IL-2), insulin, IFN-gamma, IL-4, IL-7, GM-CSF, IL-10, IL-15, TGF-beta, TNF-alpha or any other additives for the growth of cells as known to the skilled artisan. Other additives for the growth of cells include, but are not limited to, L-glutamine, surfactant, plasmanate, and reducing agents such as N-acetyl-cysteine and 2-mercaptoethanol. Exemplary media that can be used to culture T cells include RPMI 1640, DMEM, MEM, α-MEM, F-12, X-Vivo 1, X-Vivo 5, X-Vivo 15, X-Vivo 20, and Optimizer. Media can contain or be supplemented with amino acids, sodium pyruvate, and vitamins, either serum-free or supplemented with an appropriate amount of serum (or plasma) or a defined set of hormones, and/or an amount of cytokine(s) sufficient for the growth and expansion of T cells. T cells can be maintained under conditions to support growth, e.g., at an appropriate temperature (e.g., 37° C.) and atmosphere (e.g., air plus 5% CO₂), as known to those in the art.

### Methods of Administering T Cells

Once a population of T cells is isolated, stimulated and/or expanded, various methods of administering T cells to a subject may be used and are described herein. In some embodiments, the method effectively treats cancer in the subject.

A population of stimulated and/or expanded T cells and/or a depleted cell population described herein can be formulated into a cellular therapeutic. In some embodiments, a cellular therapeutic further includes a pharmaceutically acceptable carrier, diluent, and/or excipient. Pharmaceutically acceptable carriers described herein, for example, vehicles, adjuvants, excipients, and diluents, are well-known and readily available to those skilled in the art. Preferably, the pharmaceutically acceptable carrier is chemically inert to the active agent(s), e.g., a cellular therapeutic, and does not elicit any detrimental side effects or toxicity under the conditions of use.

A cellular therapeutic can be formulated for administration by any suitable route, such as, for example, intravenous, intratumoral, intraarterial, intramuscular, intraperitoneal, intrathecal, epidural, and/or subcutaneous administration routes. Preferably, the cellular therapeutic is formulated for a parenteral route of administration. In some embodiments, a cellular therapeutic is administered to a subject via an infusion (e.g., intravenous infusion).

A cellular therapeutic suitable for parenteral administration can be an aqueous or nonaqueous, isotonic sterile injection solution, which can contain anti-oxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

Dosage administered to a subject, particularly a human, will vary with the particular embodiment, the cellular therapeutic employed, the method of administration, and the particular site and subject being treated. However, a dose should be sufficient to provide a therapeutic response, e.g., immune response. A clinician skilled in the art can determine the therapeutically effective amount of a cellular therapeutic to be administered to a human or other subject in order to treat or prevent a particular medical condition. The precise amount of the cellular therapeutic required to be therapeutically effective will depend upon numerous factors, e.g., such as the specific activity of the cellular therapeutic, and the route of administration, in addition to many subject-specific considerations, which are within those of skill in the art.

Any suitable number of cells described herein can be administered to a subject. While a single therapeutic cell described herein is capable of expanding and providing a therapeutic benefit, in some embodiments, 10² or more, e.g., 10³ or more, 10⁴ or more, 10⁵ or more, or 10⁸ or more, therapeutic cells are administered as a cellular therapeutic. Alternatively, or additionally 10¹² or less, e.g., 10¹¹ or less, 10⁹ or less, 10⁷ or less, or 10⁵ or less, therapeutic cells described herein are administered to a subject as a cellular therapeutic. In some embodiments, 10²-10⁵, 10⁴-10⁷, 10³-10⁹, or 10⁵-10¹⁰ therapeutic cells described herein are administered as a cellular therapeutic.

A dose of a cellular therapeutic described herein can be administered to a mammal at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of a cellular therapeutic may be administered in a single daily dose, or the total daily dosage may be administered in two, three, four, or more divided doses administered daily, as needed. In some embodiments, a cellular therapeutic is administered via infusion. In some embodiments, a cellular therapeutic is administered in combination with checkpoint blockade, one or more cytokines such as IL-2 OR IL-7 (coincident, prior or after), or after in vivo ablation therapies such as fludarabine and cyclophosphamide.

### Methods of Measuring Change in Lymphocyte Responses

The stimulation of an immune response or of a lymphocyte may be determined by measuring the change in lymphocyte response to one or more antigens.

In some embodiments, lymphocyte response may be measured at a cellular level. In some embodiments, lymphocyte response may be measured by performing assays to measure the level of certain immune mediators. Assays may include but are not limited to the antigen presentation assays described previously. Immune mediators measured may be known immune mediators and immune mediators described herein, for example, cytokines. An exemplary assay to measure lymphocyte responses may be an assay that uses an enzyme-linked immunosorbent assay (ELISA) technique, such as an ELISPOT assay. Assays may also include analysis of upregulation of cell surface molecules such as co-stimulatory molecules (i.e. CD28, LFA-1, CD137 [4-1BB], CD154 [CD40L]), effector memory markers (i.e. CD45RO, CD62L), or HLA molecules by flow cytometry. Assays may also include evaluation of beneficial genes via gene chip analyses, or evaluation of gene expression by molecular profiling, e.g., real-time PCR.

At a cellular level, stimulation of immune responses or of a lymphocyte may be determined by the percent change in cytokine secretion in response to an identified antigen compared to a control level where the antigen is not presented, for example, by more than 5%, 6%, 7%, 8%, 9%, 10%, 20%. A control level may be without presentation of an antigen or without the addition of a composition to induce stimulation of an immune response. Stimulation of an immune response may be determined by a change in levels of immune mediators in response to an antigen presented alone compared to an antigen presented in combination with an adjuvant. Stimulation of an immune response may be determined by a change in levels of one or more immune mediators over time, for example, by more than 5%, 6%, 7%, 8%, 9%, 10%, or 20%. In some embodiments, stimulation of an immune response or of a lymphocyte may be determined by a change in the levels of different immune mediators produced by a lymphocyte, or the change in the predominant type of immune mediator produced by a lymphocyte, in response to the presentation of an antigen. For example, the change in expression and/or secretion of IL-10 to IFN-gamma may indicate stimulation of an immunostimulatory response.

At the tissue level, an immune response may be measured by the pathology of a tissue in a subject. In some embodiments, RECIST criteria (http://recist.eortc.org/publications/) can be used to determine if the tumors shrink, grow, or stay the same. In some embodiments, pathologies characterizing tumors as may be used to characterize an immune response over time and can include tumor size, altered expression of genetic markers, invasion of adjacent organs and/or lymph nodes by tumor cells. In some embodiments, immune response may be evidenced by the size of a tumor, using a metric such as tumor area and/or volume. Tumor area and/or volume may be measured over time and immune response may be indicated by the change in size and/or growth kinetics of the tumor. In some embodiments, a change in tumor size or rate of growth in a subject immunized with an immunogenic composition may be compared to the change in tumor size or rate of growth in an un-immunized control subject. In some embodiments, infiltration of the tumors with immune cells can be monitored with multi-parameter immunohistochemistry, T cell receptor sequencing, or evaluation of enriched tumor infiltrating lymphocytes using conventional immunoassays. Stimulation of immune responses or of lymphocytes can be determined by an increase in tumor infiltration by T cells.

Stimulation of immune responses or of lymphocytes at a tissue level may be determined by a change in the growth of a tumor over time in a subject immunized with antigen compared to a control, for example, by more than 5%, 6%, 7%, 8%, 9%, 10%, or 20%. Stimulation of lymphocytes at a tissue level may be demonstrated by a difference in tumor area or volume in a subject treated with antigen compared to a control for example that is more than %, 6%, 7%, 8%, 9%, 10%, or 20%. A control level may be without presentation of an antigen or without the addition of a composition to induce stimulation of an immune response.

Stimulation of immune responses or of lymphocytes at a tissue or systemic level may be determined by evaluation of the diversity, clonality, persistence, and other features of the T cell receptor (TCR) repertoire via TCR sequencing.

T cell receptors (“TCRs”) are complexes of several polypeptides that are able to bind an antigen when expressed on the surface of a cell, such as a T lymphocyte. The α and β chains, or subunits, form a dimer that is independently capable of antigen binding. The α and β subunits typically comprise a constant domain and a variable domain.

A T cell receptor includes a complex of polypeptides comprising a T cell receptor α subunit and a T cell receptor β subunit. The α and β subunits may be native, full-length polypeptides, or may be modified in some way, provided that the T cell receptor retains the ability to bind antigen. For example, the α and β subunits may be amino acid sequence variants, including substitution, addition and deletion mutants. They may also be chimeric subunits that comprise, for example, the variable regions from one organism and the constant regions from a different organism.

T cells play the role of central organizer of the immune response by recognizing antigens through T cell receptors (TCR). The specificity of a T cell depends on the sequence of its T cell receptor. The genetic template for this receptor is created during T cell development in the thymus by the V(D)J DNA rearrangement process, which imparts a unique antigen specificity upon each TCR. The TCR plays an essential role in T cell function, development and survival.

In some embodiments, T cells derived from non-specific, heterogeneous populations can be converted into T cells capable of responding to protein antigens and tumor tissues. In some embodiments, an antigen-specific T cell is characterized by the ability of the TCR of a T cell to recognize at least one antigen (e.g., a tumor antigen). Antigen-specific T cells can include e.g., cytotoxic T cells, assisted T cells, natural killer T cells, gamma delta T cells, regulatory T cells and memory T cells or more, but may be preferably memory T cells.

In some embodiments, after successful stimulation of immune responses or of lymphocytes, the diversity of the TCR repertoire or the clonality of the TCR repertoire may increase. In other cases, the persistence of a TCR clonotype may indicate T cell engraftment and establishment of a long-term immune response.

### Methods of Measuring Immune Control of Tumors

Whether an immune response impairs or enhances immune control of a tumor or cancer cell can be measured and/or characterized according to particular criteria. In certain embodiments, such criteria can include clinical criteria and/or objective criteria. In certain embodiments, techniques for assessing response can include, but are not limited to, clinical examination, positron emission tomography, chest X-ray, CT scan, MM, ultrasound, endoscopy, laparoscopy, presence or level of a particular marker in a sample, cytology, and/or histology. A positive response, a negative response, and/or no response, of a tumor can be assessed by ones skilled in the art using a variety of established techniques for assessing such response, including, for example, for determining one or more of tumor burden, tumor size, tumor stage, etc. Methods and guidelines for assessing response to treatment are discussed in Therasse et al., J. Natl. Cancer Inst., 2000, 92(3):205-216; and Seymour et al., Lancet Oncol., 2017, 18:e143-52.

In some embodiments, enhanced immune control of a tumor or cancer results in a measured decrease in tumor burden, tumor size, and/or tumor stage. In some embodiments, impaired immune control of a tumor or cancer does not result in a measured decrease in tumor burden, tumor size, or tumor stage. In some embodiments, impaired immune control of a tumor or cancer results in a measured increase in tumor burden, tumor size, or tumor stage.

### Cancer and Cancer Therapy

The present disclosure provides methods and systems related to subjects having or diagnosed with cancer, such as a tumor. In some embodiments, the subject has (or had) a positive clinical response to a cancer therapy or combination of therapies. In some embodiments, the subject had a spontaneous response to a cancer. In some embodiments, the subject is in partial or complete remission from cancer. In some embodiments, the subject has cleared a cancer. In some embodiments, the subject has not had a relapse, recurrence or metastasis of a cancer. In some embodiments, the subject has a positive cancer prognosis. In some embodiments, the subject has not experienced toxic responses or side effects to a cancer therapy or combination of therapies. In some embodiments, the subject has (or had) a negative clinical response to a cancer therapy or combination of therapies. In some embodiments, the subject has not cleared a cancer. In some embodiments, the subject has had a relapse, recurrence or metastasis of a cancer. In some embodiments, the subject has a negative cancer prognosis. In some embodiments, the subject has experienced toxic responses or side effects to a cancer therapy or combination of therapies.

In some embodiments, after treatment with a cellular therapeutic described herein, one or more immune responses of the subject adapts. For example, successful cancer therapy leads to a reduced level of one or more tumor antigens to which an immune response is raised.

In some embodiments, a tumor is or comprises a hematologic malignancy, including but not limited to, acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, Langerhans cell histiocytosis, multiple myeloma, or myeloproliferative neoplasms.

In some embodiments, a tumor is or comprises a solid tumor, including but not limited to breast carcinoma, a squamous cell carcinoma, a colon cancer, a head and neck cancer, ovarian cancer, a lung cancer, mesothelioma, a genitourinary cancer, a bladder cancer, a rectal cancer, a gastric cancer, a thyroid cancer, a bone cancer, a chondrosarcoma, an osteosarcoma, a pancreatic cancer, a cervical cancer, an endometrial cancer, a pancreatic cancer, a skin cancer, or an esophageal cancer.

In some particular embodiments, a tumor is or comprises an advanced tumor, and/or a refractory tumor. In some embodiments, a tumor is characterized as advanced when certain pathologies are observed in a tumor (e.g., in a tissue sample, such as a biopsy sample, obtained from a tumor) and/or when cancer patients with such tumors are typically considered not to be candidates for conventional chemotherapy. In some embodiments, pathologies characterizing tumors as advanced can include tumor size, altered expression of genetic markers, invasion of adjacent organs and/or lymph nodes by tumor cells. In some embodiments, a tumor is characterized as refractory when patients having such a tumor are resistant to one or more known therapeutic modalities (e.g., one or more conventional chemotherapy regimens) and/or when a particular patient has demonstrated resistance (e.g., lack of responsiveness) to one or more such known therapeutic modalities.

In some embodiments, a cellular therapeutic described herein can be administered in combination with a cancer therapy. The present disclosure is not limited to any specific cancer therapy, and any known or developed cancer therapy is encompassed by the present disclosure. Known cancer therapies include, e.g., administration of therapeutic cancer vaccines, chemotherapeutic agents, radiation therapy, surgical excision, chemotherapy following surgical excision of tumor, adjuvant therapy, localized hypothermia or hyperthermia, anti-tumor antibodies, immune stimulators, and anti-angiogenic agents. In some embodiments, cancer and/or adjuvant therapy includes a TLR agonist (e.g., CpG, Poly I:C, etc., see, e.g., Wittig et al., Crit. Rev. Oncol. Hematol. 94:31-44 (2015); Huen et al., Curr. Opin. Oncol. 26:237-44 (2014); Kaczanowska et al., J. Leukoc. Biol. 93:847-863 (2013)), a STING agonist (see, e.g., US20160362441; US20140329889; Fu et al., Sci. Transl. Med. 7:283ra52 (2015); and WO2014189805), a non-specific stimulus of innate immunity, and/or dendritic cells, or administration of GM-CSF, Interleukin-12, Interleukin-7, Flt-3, or other cytokines. In some embodiments, the cancer therapy is or comprises oncolytic virus therapy, e.g., talimogene leherparepvec (see, e.g., Fukuhara et al., Cancer Sci. 107:1373-1379 (2016)). In some embodiments, the cancer therapy is or comprises bi-specific antibody therapy (e.g., Choi et al., 2011 Expert Opin Biol Ther; Huehls et al., 2015, Immunol and Cell Biol). In some embodiments, the cancer therapy is or comprises cellular therapy such as chimeric antigen receptor T (CAR-T) cells, TCR-transduced T cells, dendritic cells, tumor infiltrating lymphocytes (TIL), or natural killer (NK) cells (e.g., as reviewed in Sharpe and Mount, 2015, Dis Model Mech 8:337-50).

Anti-tumor antibody therapies (i.e., therapeutic regimens that involve administration of one or more anti-tumor antibody agents) are rapidly becoming the standard of care for treatment of many tumors. Antibody agents have been designed or selected to bind to tumor antigens, particularly those expressed on tumor cell surfaces. Various review articles have been published that describe useful anti-tumor antibody agents (see, for example, Adler et al., Hematol. Oncol. Clin. North Am. 26:447-81 (2012); Li et al., Drug Discov. Ther. 7:178-84 (2013); Scott et al., Cancer Immun. 12:14 (2012); and Sliwkowski et al., Science 341:1192-1198 (2013)). The below Table 3 presents a non-comprehensive list of certain human antigens targeted by known, available antibody agents.

Certain cancer indications for which the antibody agents have been proposed to be useful:

TABLE 3

| Human | Antibody (commercial | |
|---|---|---|
| Antigen | or scientific name) | Cancer indication |
| CD2 | Siplizumab | Non-Hodgkin's Lymphoma |
| CD3 | UCHT1 | Peripheral or Cutaneous T-cell Lymphoma |
| CD4 | HuMax-CD4 | |
| CD19 | SAR3419, MEDI-551 | Diffuse Large B-cell Lymphoma |
| CD19 and CD3 or | Bispecific antibodies such as | Non-Hodgkin's Lymphoma |
| CD22 | Blinatumomab, DT2219ARL | |
| CD20 | Rituximab, Veltuzumab, | B cell malignancies (Non-Hodgkin's |
| | Tositumomab, Ofatumumab, | lymphoma, Chronic lymphocytic leukemia) |
| | Ibritumomab, Obinutuzumab, | |
| CD22 (SIGLEC2) | Inotuzumab, tetraxetan, CAT- | Chemotherapy-resistant hairy cell leukemia, |
| | 8015, DCDT2980S, Bectumomab | Hodgkin's lymphoma |
| CD30 | Brentuximab vedotin | |
| CD33 | Gemtuzumab ozogamicin | Acute myeloid leukemia |
| | (Mylotarg) | |
| CD37 | 16 | Chronic lymphocytic leukemia |
| CD38 | mumab | Multiple myeloma, hematological tumors |
| CD40 | mumab | Non-Hodgkin's lymphoma |
| CD52 | Alemtuzumab (Campath) | Chronic lymphocytic leukemia |
| CD56 (NCAM1) | Lorvotuzumab | Small Cell Lung Cancer |
| CD66e (CEA) | Labetuzumab | Breast, colon and lung tumors |
| CD70 | SGN-75 | Non-Hodgkin's lymphoma |
| CD74 | Milatuzumab | Non-Hodgkin's lymphoma |
| CD138 (SYND1) | BT062 | Multiple Myeloma |
| CD152 (CTLA-4) | Ipilimumab | Metastatic melanoma |
| CD221 (IGF1R) | AVE1642, IMC-A12, MK-0646, | Glioma, lung, breast, head and neck, |
| | R150, CP 751871 | prostate and thyroid cancer |
| CD254 (RANKL) | Denosumab | Breast and prostate carcinoma |
| CD261 (TRAILR1) | Mapatumumab | |
| CD262 (TRAILR2) | HGS-ETR2, CS-1008 | Colon, lung and pancreas tumors and |
| | | haematological malignancies |
| CD326 (Epcam) | Edrecolomab, 17-1A, IGN101, | Colon and rectal cancer, malignant ascites, |
| | Catumaxomab, Adecatumumab | epithelial tumors (breast, colon, lung) |
| CD309 (VEGFR2) | IM-2C6, CDP791 | Epithelium-derived solid tumors |
| CD319 (SLAMF7) | HuLuc63 | Multiple myeloma |
| CD340 (HER2) | Trastuzumab, Pertuzumab, Ado- | Breast cancer |
| | trastuzumab emtansine | |
| CAIX (CA9) | cG250 | Renal cell carcinoma |
| EGFR (c-erbB) | Cetuximab, Panitumumab, | Solid tumors including glioma, lung, breast, |
| | nimotuzumab and 806 | colon, and head and neck tumors |
| EPHA3 (HEK) | KB004, IIIA4 | Lung, kidney and colon tumors, melanoma, |
| | | glioma and haematological malignancies |
| Episialin | Epitumomab | Epithelial ovarian tumors |
| FAP | Sibrotuzumab and F19 | Colon, breast, lung, pancreas, and head and |
| | | neck tumors |
| HLA-DR beta | Apolizumab | Chronic lymphocytic leukemia, non- |
| | | Hodkin's lymphoma |
| FOLR-1 | Farletuzumab | Ovarian tumors |
| 5T4 | Anatumomab | Non-small cell lung cancer |
| GD3/GD2 | 3F8, ch14.18, KW-2871 | Neuroectodermal and epithelial tumors |
| gpA33 | huA33 | Colorectal carcinoma |
| GPNMB | Glembatumumab | Breast cancer |
| HER3 (ERBB3) | MM-121 | Breast, colon, lung, ovarian, and prostate |
| | | tumors |
| Integrin αVβ3 | Etaracizumab | Tumor vasculature |
| Integrin α5β1 | Volociximab | Tumor vasculature |
| Lewis-Y antigen | hu3S193, IgN311 | Breast, colon, lung and prostate tumors |
| MET (HGFR) | AMG 102, METMAB, SCH900105 | Breast, ovary and lung tumors |
| Mucin-1/CanAg | Pemtumomab, oregovomab, | Breast, colon, lung and ovarian tumors |
| | Cantuzumab | |
| PSMA | ADC, J591 | Prostate Cancer |
| Phosphatidylserine | Bavituximab | Solid tumors |
| TAG-72 | Minretumomab | Breast, colon and lung tumors |
| Tenascin | 81C6 | Glioma, breast and prostate tumours |
| VEGF | Bevacizumab | Tumor vasculature |
| PD-L1 | Avelumab | Non-small cell lung cancer, MCC |
| CD274 | Durvalumab | Non-small cell lung cancer |
| IDO enzyme | IDO inhibitors | Multiple |

In some embodiments, a cancer therapy is or comprises immune checkpoint blockade therapy (see, e.g., Martin-Liberal et al., Cancer Treat. Rev. 54:74-86 (2017); Menon et al., Cancers (Basel) 8:106 (2016)), or immune suppression blockade therapy. Certain cancer cells thrive by taking advantage of immune checkpoint pathways as a major mechanism of immune resistance, particularly with respect to T cells that are specific for tumor antigens. For example, certain cancer cells may overexpress one or more immune checkpoint proteins responsible for inhibiting a cytotoxic T cell response. Thus, immune checkpoint blockade therapy may be administered to overcome the inhibitory signals and permit and/or augment an immune attack against cancer cells. Immune checkpoint blockade therapy may facilitate immune cell responses against cancer cells by decreasing, inhibiting, or abrogating signaling by negative immune response regulators (e.g., CTLA-4). In some embodiments, a cancer therapy or may stimulate or enhance signaling of positive regulators of immune response (e.g., CD28).

Examples of immune checkpoint blockade and immune suppression blockade therapy include e.g., agents targeting one or more of A2AR, B7-H4, BTLA, CTLA-4, CD28, CD40, CD137, GITR, IDO, KIR, LAG-3, PD-1, PD-L1, OX40, TIM-3, TIGIT and VISTA. Specific examples of immune checkpoint blockade agents include the following monoclonal antibodies: ipilimumab (targets CTLA-4); tremelimumab (targets CTLA-4); atezolizumab (targets PD-L1); pembrolizumab (targets PD-1); nivolumab (targets PD-1); avelumab; durvalumab; and cemiplimab.

Specific examples of immune suppression blockade agents include: Vista (B7-H5, v-domain Ig suppressor of T cell activation) inhibitors; Lag-3 (lymphocyte-activation gene 3, CD223) inhibitors; IDO (indolemamine-pyrrole-2,3-dioxygenase-1,2) inhibitors; KIR receptor family (killer cell immunoglobulin-like receptor) inhibitors; CD47 inhibitors; and Tigit (T cell immunoreceptor with Ig and ITIM domain) inhibitors.

In some embodiments, a cancer therapy is or comprises immune activation or immune stimulator therapy. Specific, non-limiting examples of immune activators or immune stimulators include: IL-2, IL-7, IL-15, CD40 agonists; GITR (glucocorticoid-induced TNF-R-related protein, CD357) agonists; OX40 (CD134) agonists; 4-1BB (CD137) agonists; CD3 agonists; ICOS (inducible T cell stimulator); CD278 agonists; IL-2 (interleukin 2) agonists; and interferon agonists.

In some embodiment, a cancer therapy is or comprises a therapeutic cancer vaccine. Various therapeutic cancer vaccines in development have been described in, e.g., Sahin and Tureci (2018) Science 359(6382):1355-1360 Personalized vaccines for cancer immunotherapy; Ma et al., (2020) Scand J Immunol 2020; 00:e12875 Development of tumour peptide vaccines: from universalization to personalization; Hu et al., (2018) Nat Rev Immunol 18(3); 168-182 Towards personalized, tumour-specific, therapeutic vaccines for cancer. In some embodiments, cancer therapy is or comprises a combination of one or more immune checkpoint blockade agents, immune suppression blockade agents, and/or immune activators, or a combination of one or more immune checkpoint blockade agents, immune suppression blockade agents, and/or immune activators, and other cancer therapies such as therapeutic cancer vaccines.

Methods described herein can include preparing and/or providing a report, such as in electronic, web-based, or paper form. The report can include one or more outputs from a method described herein, e.g., a subject response described herein. In some embodiments, a report is generated, such as in paper or electronic form, which identifies the presence or absence of one or more tumor antigens (e.g., one or more stimulatory and/or inhibitory and/or suppressive tumor antigens, or tumor antigens to which lymphocytes are not responsive, described herein) for a cancer patient, and optionally, a recommended course of cancer therapy. In some embodiments, the report includes an identifier for the cancer patient. In one embodiment, the report is in web-based form.

In some embodiments, additionally or alternatively, a report includes information on prognosis, resistance, or potential or suggested therapeutic options. The report can include information on the likely effectiveness of a therapeutic option, the acceptability of a therapeutic option, or the advisability of applying the therapeutic option to a cancer patient, e.g., identified in the report. For example, the report can include information, or a recommendation, on the administration of a cancer therapy, e.g., the administration of a pre-selected dosage or in a pre-selected treatment regimen, e.g., in combination with one or more alternative cancer therapies, to the patient. The report can be delivered, e.g., to an entity described herein, within 7, 14, 21, 30, or 45 days from performing a method described herein. In some embodiments, the report is a personalized cancer treatment report.

In some embodiments, a report is generated to memorialize each time a cancer subject is tested using a method described herein. The cancer subject can be reevaluated at intervals, such as every month, every two months, every six months or every year, or more or less frequently, to monitor the subject for responsiveness to a cancer therapy and/or for an improvement in one or more cancer symptoms, e.g., described herein. In some embodiments, the report can record at least the treatment history of the cancer subject.

In one embodiment, the method further includes providing a report to another party. The other party can be, for example, the cancer subject, a caregiver, a physician, an oncologist, a hospital, clinic, third-party payor, insurance company or a government office.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

Methods for identifying and selecting antigens that stimulate and inhibit the immune response in a tumor environment are detailed below. In addition to identification and selection of stimulatory or inhibitory antigens, methods of making autologous adoptive cell therapies using the selected antigens are also demonstrated.

### Example 1. Identification of Stimulatory and Inhibitory Antigens Using mATLAS Screens Methods

A cohort of C57BL/6J mice bearing B16F10 tumors were euthanized and their tumors and spleens harvested. DNA obtained from pooled tumors was sequenced and analyzed for non-synonymous mutations. Over 1600 such mutations were identified, and these were synthesized as 399 bp DNA fragments centered upon the base pair change and transformed individually into E. coli bacteria expressing cLLO to build a candidate tumor antigen library. Splenocytes frozen from pooled spleens of the tumor-bearing mice were thawed, and CD8⁺ T cells were sorted using a negative selection bead kit. These were subsequently expanded with CD3/CD28 beads and IL-2 for 7 days followed by 1 day of rest after removal of beads and cytokine. Mouse APCs (RAW309 Cr.1 macrophage cell line) were cultured overnight, washed with PBS, then co-cultured with the bacterial library for 2 hours, washed with PBS, and then cultured with the non-specifically expanded and rested CD8⁺ T cells overnight. Harvested supernatant from the co-culture was tested for IFN-gamma and TNF-alpha by a custom mouse 384-well Meso Scale Discovery (MSD) electrochemiluminescence assay.

Results

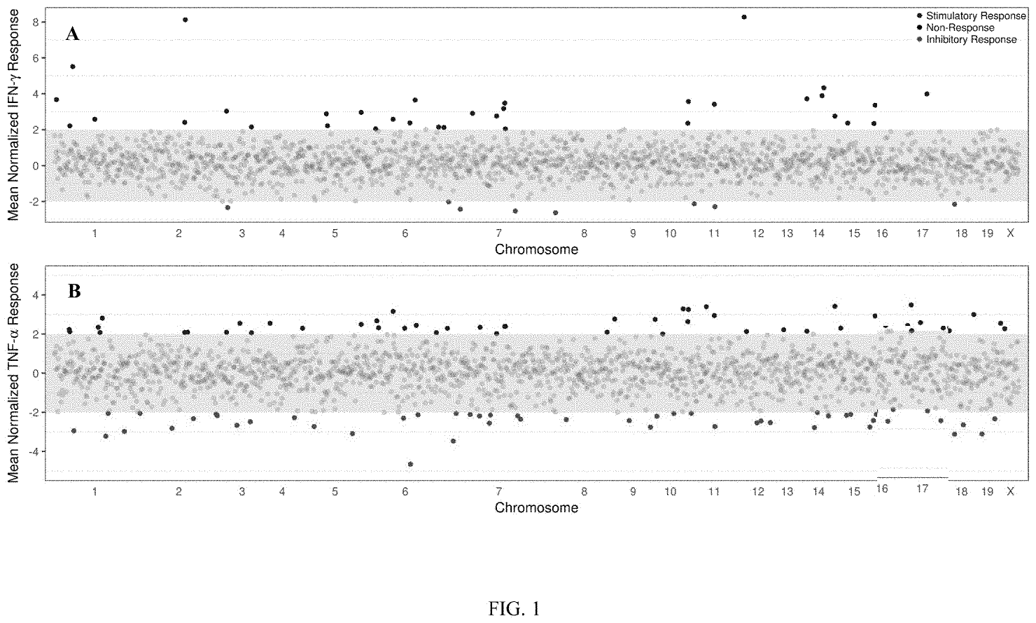

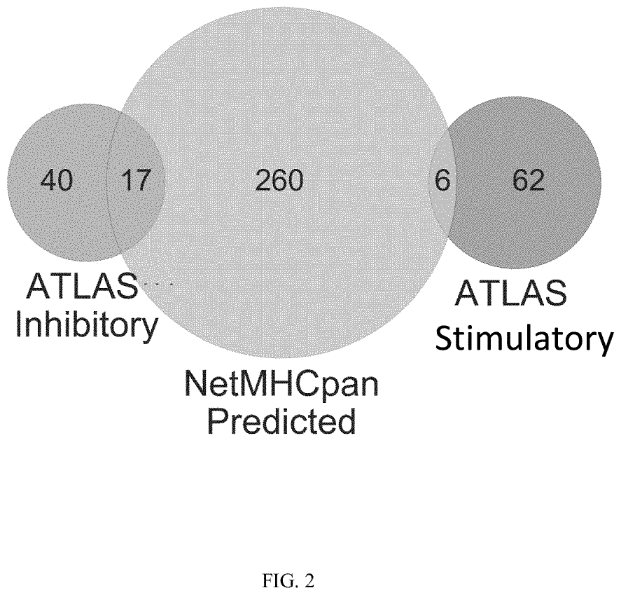

Sixty-eight antigens were identified as stimulatory (exceeding a statistical threshold above the negative control, a 399 bp fragment of the mouse actin gene) and 57 antigens were identified as inhibitory (reduced beyond a statistical threshold below the negative control), for either IFN-gamma, TNF-alpha, or both (FIG. 1). Only 2% (6 of 283) of NetMHCpan (Nielsen et al., PLoS One. 2007 Aug. 29; 2(8):e796) predicted binding antigens were empirically identified by mATLAS as stimulatory antigens. 6% (17 of 283) of NetMHCpan predicted antigens were identified by mATLAS as inhibitory antigens (FIG. 2).

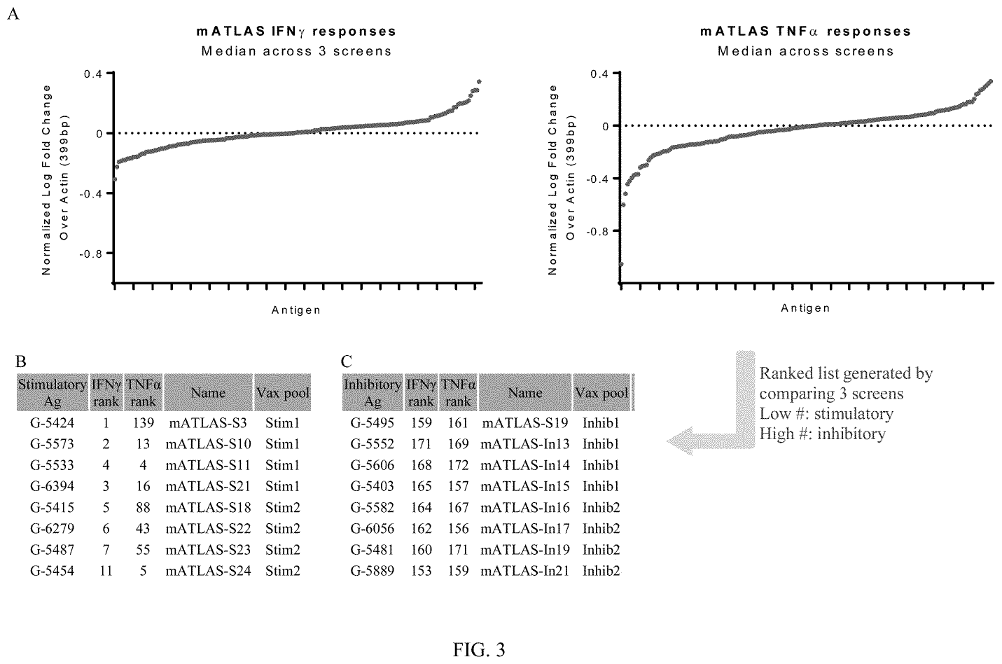

The top 50 stimulatory and 50 inhibitory antigens, and approximately 50 antigens closest to the negative control (non-responses), were used in two additional repeat mATLAS screens with increased replicates. Each antigen was ranked by its IFN-gamma signal across all 3 screens, as well as a separate rank for its TNF-alpha signal across all 3 screens. The top 10 ranked antigens (stimulatory) and 8 of the bottom 10 ranked antigens (inhibitory) were each synthesized as 27mer synthetic long peptides (SLPs) for use in mouse vaccination, as well as four 15mer overlapping peptides (OLPs) for use in ex vivo assays (FIG. 3 panels A-C).

### Example 2. Personalized Antigen-Specific T Cells for Adoptive Cell Therapy (ACT)

Aim 1: Methods to Expand ATLAS-Identified Antigen-Specific T Cells from Mouse Splenocytes:
Milestones:
   1. Identify a rapid method to expand beneficial antigen-specific T cells and confirm specificity by ELISpot.
   2. Demonstrate establishment and maintenance of a Th1 effector memory phenotype after expansion using flow cytometry.
Methods: The goal of this aim was to define optimal conditions for antigen-specific T cell expansion in mice. These methods were subsequently used to demonstrate preclinical proof of concept for an ATLAS-based ACT therapy in a B16F10 mouse tumor efficacy model (Aim 2). Published studies have previously shown the feasibility of in vitro antigen-specific T cell expansion by peptide stimulation in mice with corresponding anti-tumor efficacy when delivered by ACT [Starobinets H et al., (2018). Ex vivo ATLAS-identification of neoantigens for personalized cancer immunotherapy in mouse melanoma. American Association for Cancer Research Annual Meeting; Li et al., 2016].
   Milestone 1: To determine the optimal conditions for in vitro expansion of antigen-specific murine T cells, a combination of factors were tested. The top 8 stimulatory and inhibitory antigens identified according to Example 1 were synthesized as overlapping peptides (OLPs) 15 amino acids in length (overlapping by 11aa), spanning a 27 amino acid sequence centered upon each antigen mutation. Splenic T cells derived from B16F10 tumor-bearing mice were sorted by negative bead selection and seeded into culture with mouse APCs that had been pulsed with OLPs spanning stimulatory and/or inhibitory antigens. Published literature in mouse models demonstrate that combinations of various cytokines greatly influence the expansion and phenotype of in vitro expanded T cells [Li et al., 2016; Zoon et al., 2015]. Factors including cytokine addition (e.g., IL-2, IL-7, IL-15, IL-21), and OLP concentration were tested to maximize T cell proliferation and potential to shift inhibitory T cell responses to stimulatory responses. If sufficient beneficial antigen-specific T cells were not generated through this process, antigen-specific T cells were sorted by an activation marker such as CD137 followed by anti-CD3/CD28 non-specific expansion. T cell expansion was monitored through cell number and viability. Antigen-specific responses were assessed by ELISpot assay, meso-scale discovery (MSD), and flow cytometry.
Goal: Maximize beneficial antigen-specific T cell expansion for ACT therapy in mice (˜10⁵-10⁶ total antigen-specific T cells to up to 16 ATLAS-defined stimulatory and/or inhibitory antigens).
   Milestone 2: For successful ACT therapy, it is well established that the phenotype of transferred T cells is important. To ensure the quality of expanded antigen-specific T cells for ACT, markers of T cell activation (e.g., IFN-gamma, TNF-alpha, CD44, CD69) and T cell memory (CD44, CD62L) were assessed. Concurrent with Milestone 1, flow cytometry analysis was used to analyze expanded T cell populations to guide optimal T cell expansion conditions.
Goal: Develop mouse T cell expansion conditions for T cell activation and memory while selecting against a T cell exhaustion phenotype.

### Aim 2: Efficacy of Expanded Antigen-Specific T Cells in the B16F10 Melanoma Model

Milestones:
   1. Demonstrate efficacy of ATLAS-defined ACT across in vivo studies.
   2. Explore the efficacy of ATLAS-defined ACT in combination with checkpoint inhibition.
Preliminary data: Using a vaccine modality, ATLAS-identified stimulatory antigen candidates demonstrated significant T cell responses as well as anti-tumor efficacy against B16F10 tumor challenge in initial studies [PCT/US2019/053672, filed Sep. 27, 2019]. Strikingly, therapeutic immunization with inhibitory antigen peptides led to a marked and significant increase in tumor growth kinetics. These preliminary data demonstrated the ability of the ATLAS platform to identify and characterize desirable as well as potentially unwanted antigen-specific T cell responses in an aggressive in vivo mouse tumor model. The advantages of ATLAS antigen selection were applied in the proposed ACT therapy by selectively expanding T cells that are likely to enhance immune control of tumors and filtering out T cells that are likely to impair immune control of tumors.
Research Methods: In vivo studies were carried out to demonstrate preclinical proof of concept for ATLAS-derived T cell therapy in C57BL/6 mice using the B16F10 cell line, a highly aggressive melanoma model. Previous studies have demonstrated the feasibility of effective ACT in tumor-bearing mice as a monotherapy or in combination with checkpoint inhibitors [Mahvi D A et al. Ctla-4 blockade plus adoptive T-cell transfer promotes optimal melanoma immunity in mice. J Immunother 2015; 38:54-61. 10.1097/CJI.0000000000000064]. This study improves on existing methods through enrichment of antigen-specific T cells that target tumors for destruction.
   Milestone 1: C57BL/6 mice 6-8 weeks of age were prospectively divided into groups containing negative controls or expanded antigen-specific T cells (Aim 1) at different T cell doses (10⁵-10⁶ cells). B16F10 melanoma cells (1×10⁵ tumor cells/mouse) were injected subcutaneously to the anterior right flank. Seven days after tumor implantation, antigen-specific T cells derived as per Aim 1 were adoptively transferred intravenously to tumor-bearing mice. Efficacy was monitored kinetically using tumor measurements, flow cytometry and/or ELISpot analysis of local and systemic T cell responses.
Goal: Demonstrate more rapid tumor clearance after antigen-specific ACT compared with transfer of non-specifically expanded T cells.
   Milestone 2: Checkpoint inhibitor administration was assessed for potential synergy with the proposed ACT. ACT in combination with checkpoint inhibition has demonstrated remarkable clinical responses in some patients [Zacharakis et al., 2018]. In this study, anti-PD1 antibodies were intraperitoneally administered in the presence or absence of ATLAS-derived ACT therapy. As in Milestone 1, efficacy was monitored kinetically using tumor measurements, flow cytometry and/or ELISpot analysis of local and systemic T cell responses.
Goal: Demonstrate effect of checkpoint blockade therapy in combination with antigen-specific ACT
   Aim 3: Expansion of ATLAS-Identified Antigen-Specific Human T Cells from Peripheral Blood Mononuclear Cells
Milestones:
   1. Determine a process to expand human antigen-specific CD4⁺ and CD8⁺ T cells.
   2. Develop antigen-specific CD4⁺ and CD8⁺ T cell isolation methods.
   3. Develop methods to maintain antigen-specific CD4⁺ and CD8⁺ T cells of desirable phenotype, or re-educate to desirable phenotype.
   4. Develop a process to rapidly and non-specifically expand the antigen-specific T cells of desirable phenotype.
Preliminary data: Using ATLAS, virus-specific stimulatory antigens have been identified from human leukapheresis samples. These were used to develop methods as apheresis products from healthy human donors are readily available.
Research Methods: The goal of this aim is to develop methods for antigen-specific expansion of human T cells obtained from leukapheresis using peptides, cytokine cocktails (IL-2, IL-7, IL-15 and/or IL-21), and other agents.

As frequency of antigen-specific T cells in the blood is low, the expansion took place in several phases. The first phase specifically expanded T cells using overlapping peptides (15mers overlapping by 11 amino acids) of antigens combined with cytokines to induce proliferation. Antigen-specific cells were then sorted by T cell activation markers, and exposed to appropriate media and agents to maintain a desirable phenotype, or re-educated to a desirable phenotype. In the final phase, the enriched antigen-specific T cells of desirable phenotype underwent a rapid, non-specific expansion protocol to generate >10⁹ antigen-specific T cells suitable for administration to a patient [Gerdemann et al., 2012; Huarte et al., 2009; Wolf et al., 2014; Yee et al., 2002].

As described in preliminary data, immunodominant ATLAS-identified antigens from a range of viruses were used to expand T cells from healthy-donor PBMCs. Each milestone below was defined to optimize each phase of the T cell expansion processes in healthy donors and was subsequently verified using whole blood from cancer patients and antigen-specific T cells. Nearly 20 years ago, several groups observed that tumor-reactive T cells can be detected in the peripheral blood and these cells can be isolated and expanded while maintaining anti-tumor activity. With recent advances such as engineered CAR-T cell and TIL-based therapies for cancer, a method to identify antigens using the ATLAS platform and develop antigen-specific T cell therapy with peptides is feasible. However, unlike CAR-T cells which need an actionable target on all tumor cells and TIL therapies which often generate T cells of a single specificity and subset, Applicant's approach generated CD4⁺ and CD8⁺ T cells of broad specificities, increasing the likelihood of tumor eradication and the potential to limit metastatic tumor escape.
Milestone 1: To determine the basic conditions for antigen-specific T cell expansion, three factors were assessed: 1) antigen presenting cells (APCs), 2) CD4⁺ and CD8⁺ co-culture and 3) pooled or individual antigen stimulation using a single defined T cell medium and peptide concentration. The use of professional APCs such as dendritic cells to present peptides was compared to direct stimulation of peripheral blood mononuclear cells (PBMCs). While professional APCs are optimal for antigen presentation, use of minimally manipulated PBMCs was more practical and less complex than sorting and deriving dendritic cells from CD14⁺ monocytes. The presence of multiple APC subtypes, including non-professional APCs, in PBMCs (e.g., B cells, monocytes and macrophages) made this approach feasible. Once the source of APCs was defined, antigen-specific CD4⁺ and CD8⁺ T cells were expanded in co-culture, or alternatively cultured independently. (CD4+ T cells expand more rapidly than CD8⁺ T cells and as a result may dominate the culture if grown together). Optimal cytokine requirements for proliferation and survival were determined for T cell subsets. To address concerns that peptide pooling induces antigen competition, antigen pooling was compared to single antigen stimulation. In addition, comparisons of stimulatory and inhibitory peptides, separately or combined, were performed, with the goal of re-educating inhibitory T cells to respond in a beneficial way (i.e. immune control of tumors).
Once initial conditions for expansion were determined, multiple parameters were evaluated to determine maximal expansion: 1) T cell expansion media, 2) cytokine and other agent combinations to induce proliferation, preferably maintaining a naïve or central memory phenotype, or inducing a desirable activated effector phenotype, 3) peptide concentration, and 4) starting cell concentration. To monitor the effectiveness of T cell expansions, cell numbers and viability were assessed throughout the expansion culture. Antigen-specific responses were monitored by cytokine secretion in response to antigen stimulation and by a flow cytometry-based panel of activation and exhaustion markers to identify the phenotype of the T cells.
Goal: Identification of culture conditions that yield an increase in the number of beneficial antigen-specific CD4⁺ and CD8⁺ T cells that maintain a naïve or central memory phenotype, or a desirable activated effector phenotype (i.e., that enhances immune control of tumors), without pushing T cells to exhaustion.
Milestone 2: The objective of this milestone was to determine a suitable strategy for isolation of expanded antigen-specific T cells developed under Milestone 1. Expanded T cells are sorted using an antigen-specific activation marker. Activation markers are expressed on T cells after antigen recognition. Antibodies were used to label the activation markers 4-1BB (CD137), IL-2R (CD25) and CD40L (CD154) on pooled or individual CD4⁺ and CD8⁺ T cell subsets and capture activated cells using Miltenyi microbead reagents and magnetic columns. The purity of antigen-specific T cell populations before and after isolation was assessed by ELISpot or intracellular cytokine staining assays. A purity of >80% antigen-specificity is desired. If activation markers did not isolate T cells sufficiently, alternative approaches such as additional activation markers, use of IFN-gamma cytokine capture systems, or flow cytometry-based sorting methods, were used. For some purposes, it was desirable to isolate under Milestone 2 only T cells responsive to inhibitory antigens. T cells responsive to inhibitory antigens may be discarded at this stage.
Goal: ≥80% purity of beneficial antigen-specific T cells.
Milestone 3: The objective of this milestone was to develop methods to maintain antigen-specific CD4+ and CD8+ T cells of desirable phenotype (i.e., that enhances immune control of tumors), or re-educate from an undesirable phenotype (i.e., that impairs immune control of tumors), to a desirable phenotype (i.e., that enhances immune control of tumors). Isolated T cells from Milestone 2 were incubated with cytokines and other agents to determine stability or plasticity of phenotype. Combinations were optimized to 1) maintain a desirable activated effector phenotype, and 2) re-educate from an undesirable phenotype to a desirable activated effector phenotype. Isolated T cells responsive to inhibitory antigens were re-educated either in the presence of, or separately from, T cells responsive to stimulatory antigens. Separately re-educated T cells may be recombined with T cells responsive to stimulatory antigens prior to non-specific expansion below. In some instances, only T cells responsive to stimulatory antigens were non-specifically expanded.
Milestone 4: The objective of this milestone was to develop a rapid non-specific expansion process of isolated antigen-specific T cells of Milestone 3 to achieve a cell number of up to 10×10⁹ antigen-specific cells. T cells were added to G-Rex closed culture flasks and activated with either CD3/CD28 magnetic beads or CD3/CD28/CD2 soluble antibodies to promote non-specific expansion of T cells. The effect of growth media, activator concentration, pro-proliferative and pro-survival cytokine combinations (IL-2, IL-7, IL-15 and IL-21) and the addition of irradiated PBMCs to the culture was tested. Cells were assessed for growth rate, viability and T cell phenotype by flow cytometry, including memory, activation and exhaustion markers.
Goal: Define conditions that achieve maximal antigen-specific T cell proliferation while maintaining a desirable activated effector or central memory phenotype (i.e., that enhances immune control of tumors), and retain viability >70%.

### Example 3. Adoptive Transfer of In Vivo-Primed, Antigen-Specific T Cells in the BJ 6F10 Mouse Melanoma Model

ATLAS methods were extended to adoptive cell therapy by selectively expanding in vivo, through vaccination with vaccines comprising ATLAS-identified antigens, T cells that are likely to enhance immune control of tumors, or conversely, to impair immune control of tumors.

### Goals:

1. Demonstrate anti-tumor efficacy of adoptive cell therapy using T cells enriched from tumor-bearing mice that have been immunized with a vaccine containing a protective ATLAS-identified antigen into naïve tumor-bearing mice
2. Explore pro-tumor effect of adoptive cell therapy using T cells enriched from tumor bearing mice that have been immunized with a vaccine containing a deleterious ATLAS-identified antigen into naïve tumor-bearing mice

### Preliminary Data:

A vaccine comprising ATLAS-identified stimulatory antigens elicited significant T cell responses and showed anti-tumor efficacy against B16F10 tumor challenge in mouse studies [PCT/US2019/053672, filed Sep. 27, 2019]. Strikingly, therapeutic immunization with inhibitory antigen peptides led to a marked and significant increase in tumor growth kinetics. These data demonstrate the ability of the ATLAS platform to identify and characterize desirable, as well as potentially unwanted, antigen-specific T cell responses in an aggressive in vivo mouse tumor model.

### Design:

In vivo studies were carried out to demonstrate pre-clinical proof of concept for ATLAS-derived T cell therapy in C57BL/6 mice using the B16F10 cell line, a highly aggressive melanoma model. Previous studies had demonstrated the feasibility of effective adoptive cell therapy in tumor-bearing mice as a monotherapy or in combination with checkpoint inhibitors [Mahvi D A et al. Ctla-4 blockade plus adoptive T-cell transfer promotes optimal melanoma immunity in mice. J Immunother 2015; 38:54-61. 10.1097/CJI.0000000000000064].

Briefly, C57BL/6 Thy1.2 mice 6-8 weeks of age were injected subcutaneously in the anterior right flank with B16F10 melanoma cells (1×10⁵ tumor cells/mouse). Three days after tumor implantation, mice were immunized with a protective vaccine comprising 2 previously known efficacious B16F10 antigens (M30+Trp2) or a deleterious vaccine comprising an inhibitory antigen identified in Example 1 together with the 2 previously known B16F10 antigens (Mmp9FS+M30+Trp2) formulated with a triple adjuvant combination (CpG, 3D-PHAD, synthetic saponin) or adjuvant alone, and then boosted twice more on days 10 and 17. On Day 20, mice were euthanized, and their draining lymph nodes and spleens harvested and pooled within groups. For each group, T cells were sorted using magnetic beads (CD3+), and then further separated into the CD4⁺ and CD8⁺ T cell subsets. In parallel, on day 14, a new group of C57BL/6 Thy1.1 mice 6-8 weeks of age were injected subcutaneously in the anterior right flank with B16F10 melanoma cells (1×10⁵ tumor cells/mouse). On D20 (D6 post tumor implantation into the 2^{nd} group of mice), the Thy1.1 mice were randomized into nine groups, and the CD3⁺, CD4⁺ or CD8⁺ T cells sorted from the immunized Thy1.2 mice were adoptively transferred intravenously. Efficacy was monitored kinetically using tumor measurements, flow cytometry and/or ELISpot analysis of local and systemic T cell responses.

### Results:

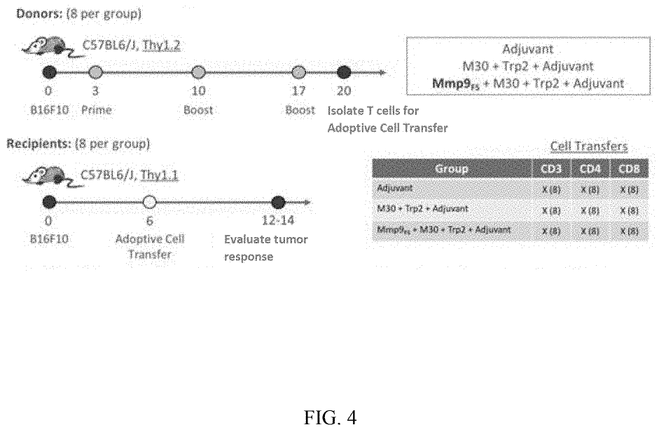

FIG. 4, Panel A is a diagram of methods to show effects of adoptive cell therapy on tumor progression, using T cell primed in vivo primed by vaccination with stimulatory or inhibitory antigens, followed by isolation of T cells and adoptive transfer to B16F10 tumor-bearing mice.

### Example 4. Methods of Making T Cell Compositions for Autologous Adoptive Cell Therapy (GEN-011)

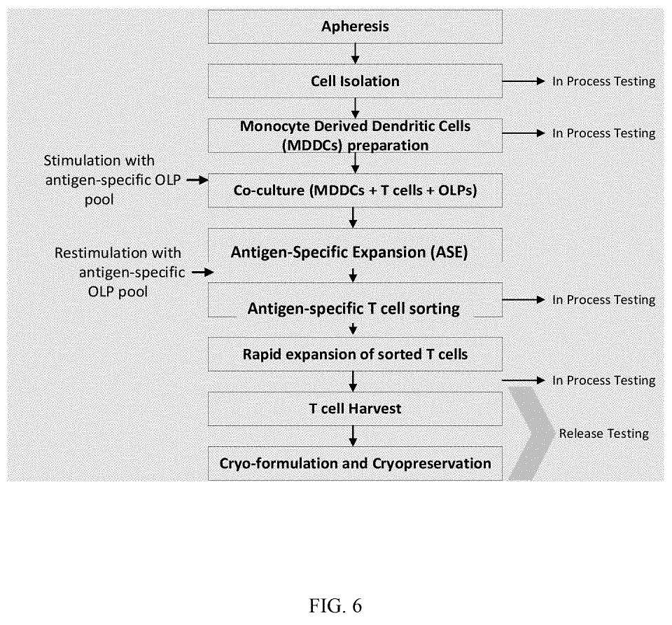

FIG. 6 summarizes the manufacturing process for autologous adoptive cell therapy GEN-011 drug substance and drug product. Initial process development was performed using healthy donors and model viral antigens. Subsequently, process development activities were performed using autologous cells and antigens specific to cancer patients.

Key manufacturing steps in the GEN-011 process include: i) acquisition and processing of a patient apheresis unit to enrich CD14⁺ monocytes and T cells; ii) differentiation of monocytes into mature dendritic cells (MDDCs); iii) antigen-specific stimulation and expansion (ASE) of isolated T cells; iv) sorting to isolate the antigen-specifically stimulated and expanded T cells; v) rapid non-specific expansion of the sorted T cells; vi) harvesting, washing, and cryoformulation of the rapidly expanded T cells; and vii) cryopreservation, yielding the autologous adoptive cell therapy GEN-011 drug product. Each of these steps is briefly described in the following sections.

### Processing of Patient Apheresis Material and Isolation of Monocytes and T Cell Populations

Starting material for the GEN-011 manufacturing process consisted of a freshly collected apheresis unit isolated from each patient enrolled in the GEN-011 clinical trial. The fresh apheresis material was shipped overnight to the GMP manufacturing site and processed in a GMP suite using aseptic procedures. On the day of receipt, the apheresis material was processed for CD14⁺ monocyte isolation using CD14 microbeads and an automated cell separation instrument (CliniMACS Plus, Miltenyi Biotec, or equivalent instrument). CD14⁺ cells were further washed, cryoformulated in cryobags, and cryopreserved using controlled rate freezing and stored at <−150° C. Next, CD45RO⁺ T cells were isolated from the CD14⁻ cell sub-population using anti-CD45RO biotinylated antibody followed by anti-biotin microbeads (Miltenyi Biotec) and sorting on the automated cell separation instrument. Alternatively, CD4⁺ and CD8⁺ T cells were isolated using CD4/CD8 microbeads (Miltenyi Biotec) and sorting on the automated cell separation instrument. The T cell populations were further washed and cryopreserved. A fraction of cryopreserved monocytes and T cells were shipped at <−150° C. via Cryoport to Applicant's premises. ATLAS screening was performed to identify the patient's antigen-specific T cell responses and select stimulatory antigens (see section below, ATLAS Identification and Selection of Antigens; Generation of OLP Pools). Remaining cryopreserved monocytes and T cells were stored at <−150° C. at the GMP facility until initiation of the MDDC preparation.

Differentiation of Monocytes into Monocyte-Derived Dendritic Cells (MDDCs)

CD14⁺ monocytes were differentiated and subsequently matured into monocyte-derived dendritic cells (MDDCs) using the ImmunoCult Dendritic Cell Culture Kit (StemCell Technologies). The kit contains: (i) serum-free and animal component-free dendritic cell growth medium; (ii) Differentiation Supplement optimized for the in vivo culture and differentiation of human monocytes into immature DCs; and (iii) Maturation Supplement to support maturation of immature DCs to mature DCs. The kit allows for high yields of mature DCs expressing high levels of HLA-DR and the co-stimulatory molecules, CD83 and CD86.

CD14⁺ monocytes, cryopreserved in cryobags, were thawed, washed to remove cryoprotectant, and then resuspended in the dendritic cell growth medium and Differentiation Supplement under aseptic culture conditions. Cells were cultured in a 37° C. incubator containing 5% CO₂ for 6±2 days. Thereafter, the Maturation Supplement was added, and the cells were cultured for 2±1 more days. The mature MDDCs were harvested, and medium exchange was performed prior to co-culture with T cells for antigen-specific stimulation and expansion.

### ATLAS Identification and Selection of Antigens; Generation of OLP Pools

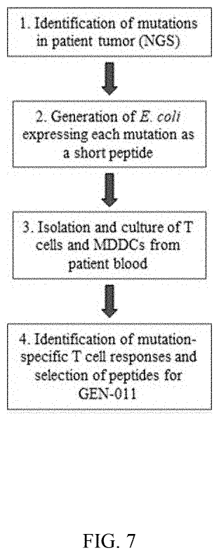

ATLAS screening was performed to identify each patient's antigen-specific T cell responses and select stimulatory tumor antigens according to the methods of WO 2018/175505, the contents of which are incorporated herein by reference in their entirety. An overview of the ATLAS method is provided in FIG. 7.

Briefly, next-generation sequencing (NGS) enabled the identification of genomic variant, fusion events, or viral sequences within a patient's tumor that are specific to the tumor cells and not found within the patient's germline DNA or RNA sequences. These mutations were subsequently screened using Applicant's ATLAS method, which allows rapid, high-throughput identification of pre-existing antigen-specific T cell responses to each tumor-specific mutation, without the use of in silico down-selection criteria. With the ATLAS method, a full complement of putative polypeptide antigens is expressed as individual clones in bacterial hosts (Escherichia coli [E. coli]), which are co-cultured with autologous professional and/or non-professional antigen presenting cells (APCs), or cells derived from the patient's blood monocytes (monocyte-derived dendritic cells (MDDCs)). As the MDDCs ingest and process the E. coli-enclosed polypeptide library, they present peptide epitopes in the context of HLA class I or II molecules that can be recognized by T cells derived from the same patient. If recognition events occur, a readout of T cell activation (or inhibition) can be measured by the secretion of cytokines such as IFN-gamma. Antigens that elicit T cell activation (or inhibition) were then selected (or de-selected) for further uses.

The stimulatory tumor antigens identified and selected by ATLAS methods for each patient were synthesized using solid phase peptide synthesis as 4 overlapping peptides (OLPs) of 15 amino acids in length, with an 11 amino acid overlap (see Table 4). The OLPs were pooled into an OLP pool unique to the patient. Each OLP pool consisted of up to 120 individual OLPs. The OLPs are not contained in the final autologous adoptive cell therapy GEN-011 drug product, but were used for ex vivo peptide-mediated stimulation and expansion of the patient's autologous T cells. In multiple studies of peptide mixes with various lengths, OLPs of 15mers overlapping by 11 amino acids were found to efficiently stimulate both CD4⁺ and CD8⁺ T cell responses.

TABLE 4
An example of the 4 OLPs (overlapping peptides), with each OLP being
a 15mer in length, derived from a single ATLAS-identified and selected
tumor antigen. Underline indicates mutation position.

| OLP | Amino acid Sequence |
|---|---|
| OLP-1-1 | aa1-aa2-aa3-aa4-aa5-aa6-aa7-aa8-aa9-aa10-aa11-aa12-aa13-aa14-aa15 |
| OLP-1-2 | aa5-aa6-aa7-aa8-aa9-aa10-aa11-aa12-aa13-aa14-aa15-aa16-aa17-aa18-aa19 |
| OLP-1-3 | aa9-aa10-aa11-aa12-aa13-aa14-aa15-aa16-aa17-aa18-aa19-aa20-aa21-aa22-aa23 |
| OLP-1-4 | aa13-aa14-aa15-aa16-aa17-aa18-aa19-aa20-aa21-aa22-aa23-aa24-aa25-aa26-aa27 |

### Antigen-Specific Stimulation and Expansion (ASE) of the Isolated T Cells

The cryopreserved T cells were thawed, the cryoprotectant was removed via medium exchange, and the cells were resuspended in serum-free T cell culture medium (OpTmizer, containing Immune Cell SR; Gibco). Cells were co-cultured with the above-prepared MDDCs and the same patient-specific OLP pool at 2 μg/mL per OLP (range: 0.5-10 μg/mL) in a G-Rex or equivalent tissue culture vessel for up to 10±2 days in a 37° C. incubator containing 5% CO₂. A MDDC to T cell ratio of 1:8±4 during the antigen-specific stimulation and expansion culture period was employed. The medium was supplemented every 2-3 days with combinations of cytokines, including but not limited to 12.5 IU/mL IL-2 (range: 8-20 IU/mL), 5 ng/mL IL-7 (range: 1-20 ng/mL), 1 ng/mL IL-15 (range: 1-20 ng/mL), and 1 ng/mL IL-21 (range: 1-20 ng/mL).

### Sorting of Cultured Cells to Isolate Antigen-Specifically Stimulated Cells

Upon completion of ASE, partial medium exchange was carried out and fresh serum-free T cell culture medium and the same patient-specific OLP pool (target: 6 μg/mL each peptide; range: 1-12 μg/mL) was added for re-stimulation. The culture was incubated with anti-CD154-biotin antibody (BioLegend) for antigen surface trapping and maximum recovery, followed by anti-CD137-biotin antibody, and then finally, anti-biotin microbeads (Miltneyi Biotec), to facilitate selection and isolation of antigen-specific cells expressing the CD154 and/or CD137 activation markers using an automated cell separation instrument. (Alternatively, the culture was incubated with anti-CD137-biotin antibody only, then anti-biotin microbeads for selection and isolation of antigen-specific cells expressing the CD137 marker.) CD137 is more highly expressed on CD8⁺ T cells while CD154 is mainly expressed on CD4⁺ T cells. T cell isolation based on antibodies to both activation markers ensures that both CD4⁺ and CD8⁺ T cells are highly enriched; however, selection using only anti-CD137-biotin antibody yields sufficient quantities of both CD4⁺ and CD8⁺ T cells. The isolated CD154⁺ and/or CD137⁺ antigen-specifically stimulated cells were resuspended in serum-free T cell culture medium to initiate further expansion.

### Rapid Non-Specific Stimulation and Expansion

The isolated antigen-specific, CD154⁺ and/or CD137⁺ T cells were transferred to the rapid non-specific stimulation and expansion phase (REP) of the process, with a duration of 10±4 days. At this stage, the isolated cells were non-specifically stimulated and expanded using either anti-CD3 and CD28 monoclonal antibodies (T Cell TransACT, Miltenyi Biotec) or anti-CD3, CD28, and CD2 monoclonal antibodies (ImmunoCult, StemCell Technologies), and then seeded into a G-Rex or equivalent tissue culture vessel and cultured in a 37° C. incubator containing 5% CO₂. The tissue culture medium was comprised of serum-free T cell culture medium, supplemented with 20 ng/mL IL-7 (range: 1-30 ng/mL), 15 ng/mL IL-15 (range: 1-20 ng/mL), and 30 ng/mL IL-21 (range: 1-40 ng/mL).

### Harvest, Wash, Formulation, and Cryopreservation of Expanded Cells

Following completion of the rapid non-specific stimulation and expansion phase, the T cells were harvested from culture, washed from the culture medium, and resuspended in diluent (Plasmalyte, Baxter) to constitute the GEN-011 drug substance. A dilution factor protocol was employed to formulate the drug substance into Human Serum Albumin (HSA; Octapharma) and cryoprotectant (CryoStor CS10, BioLife Solutions).

The final formulation of the GEN-011 drug product was a mixture of diluent, HSA and cryoprotectant (˜5% DMSO final). All the components of the final formulation were manufactured to cGMP standard. The final formulated GEN-011 drug product was filled in cryobags and stored viably frozen at <−150° C. until use.

### Example 5: Starting Material: Processing of Apheresis Product for Cell Isolation

Starting material for the GEN-011 manufacturing process consists of a freshly collected patient's apheresis. To mimic patient material collection, a healthy donor's apheresis product was collected and shipped overnight to a Contract Development and Manufacturing Organization (CDMO). The apheresis product was split into two fractions to evaluate the impact of any shipping-related delays. One fraction was processed the next day (˜24 h) and the second fraction was processed at 2 days (˜48 h) after apheresis for isolation of monocytes and T cells using magnetic bead-coupled antibodies and a CliniMACS automated cell separation instrument. Sorted cells were immediately cryopreserved. Sorted CD14⁺ monocyte cells were washed, cryoformulated and cryopreserved. Two separate strategies for T cell enrichment were explored: i) positive selection with a combination of CD4⁺ and CD8⁺ microbeads; or ii) the preferential enrichment of memory T cells by positive selection with CD45RO microbeads. Data in Table 5 show the representative results from CD14⁺ monocytes and CD4⁺/CD8⁺ microbeads (“CD3⁺ cells” in the table) processed and frozen both 24- and 48-hours post-apheresis. Based on comparable yields and viability, cell processing and freezing can occur up to 48 h post apheresis.

TABLE 5
Impact of apheresis hold time for isolation of
CD14⁺ monocytes and T cell populations (pre-expansion)

| | Time (hrs post | |
|---|---|---|
| | apheresis | |
| Apheresis cell #s | 0 | 1.33e10 |
| Apheresis % | | 99.4 |
| viability | | |

| | | Starting | CD14+ cells | CD3+ cells |
|---|---|---|---|---|
| | | apheresis | (post CD14 | (post CD4/8 |
| | | cell #s | sort) | sort) |
| Cell #s (yield) | 24 | 5.13e9 | 5.8e8 | 2.3e9 |
| % viability | | | 96.9 | 96.1 |
| % purity | | | 93 | 93 |
| Cell #s (yield) | 48 | 6.08e9 | 7.4e8 | 1.06e9 |
| % viability | | | 97.1 | 98.3 |
| % purity | | | 93 | 96 |

### Example 6: MDDCs Improve Antigen-Specific Stimulation and Expansion of T Cells

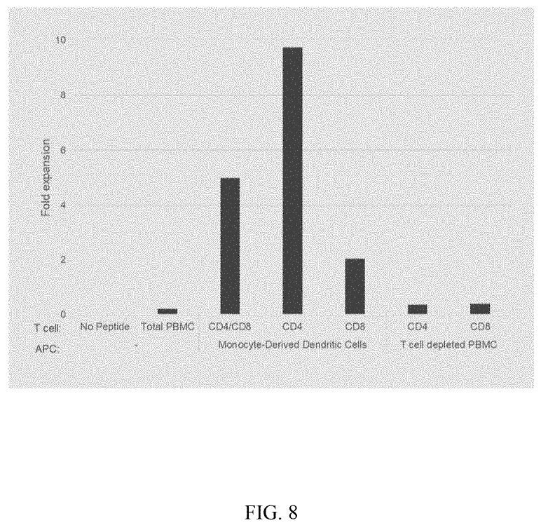

The use of professional APCs, called MDDCs, to present peptides to T cells was compared with cross-presentation by T cell-depleted PBMCs. Minimally manipulated PBMCs may be more practical for manufacturing than deriving dendritic cells from CD14⁺ monocytes; however, professional APCs are useful for: i) antigen presentation as they are up to 1000-fold more efficient in stimulating resting T cells; ii) providing optimal co-stimulatory signals for full T cell stimulation; and iii) secretion of IL-12, which polarizes T cells towards a beneficial Th1 phenotype and increases anti-tumor immune responses. Experiments showed that culturing sorted CD4⁺ and/or CD8⁺ T cells with model peptide antigens (of viral origin) and MDDCs drove significant (10-fold) antigen-specific stimulation and expansion of T cells, compared to culturing with total PBMCs (<1-fold) or T cell-depleted PBMCs (<1-fold) (FIG. 8). Fold expansion of viable cells after 10-day expansion is shown.

### Example 7: Differentiation and Maturation of MDDCs

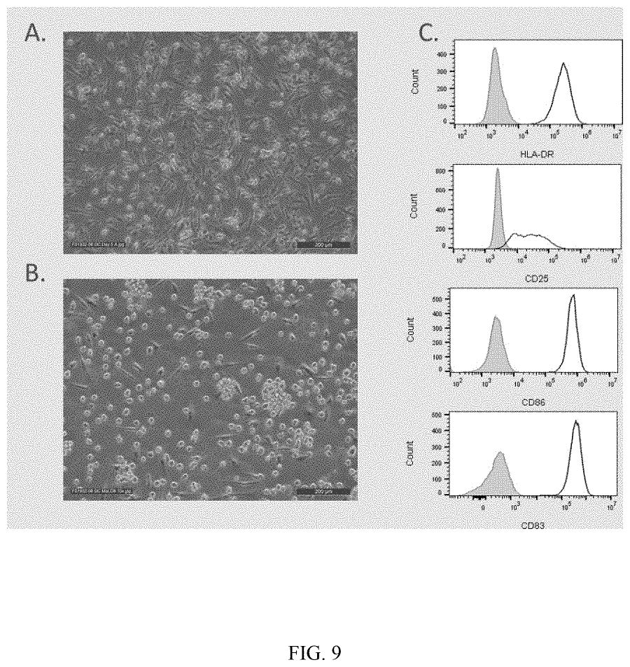

CD14⁺ monocytes were differentiated and matured in vitro into MDDCs, using the ImmunoCult Dendritic Cell Culture kit (StemCell Technologies). This kit has been optimized for high yields and MDDC viability. Harvested MDDCs were mature, as shown by upregulation in expression of typical maturation markers, HLA-DR, CD25, CD86 and CD83 (FIG. 9).

Panel A is an image of differentiated immature dendritic cells (DCs) at Day 6, showing numerous dendrites, a hallmark of DCs. Panel B is an image of mature DCs at Day 8 (harvest). The cells have rounded up and are easily accessible for harvest. Panel C shows histograms of typical MDDC maturation markers at Day 8. Grey shaded: Isotype controls, Black lines: specific antibodies.

### Example 8: Co-Culture of MDDCs with T Cells

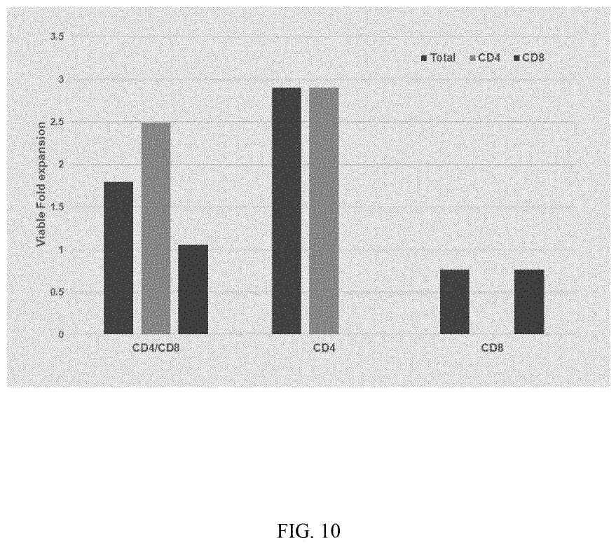

The impact of CD4⁺ and CD8⁺ T cell co-culture on the individual expansion of each T cell subset was examined. T cells were expanded for 10 days in the presence of MDDCs pulsed with model peptide antigens (of viral origin) as separate CD4⁺ or CD8⁺ T cell cultures, or in a combined CD4⁺/CD8⁺ T cell culture. Similar fold-expansion was observed from the CD4⁺/CD8⁺ T cell co-culture as compared to individual CD4⁺ or CD8⁺ T cell cultures (FIG. 10), indicating that the presence of one subset does not significantly impact the growth of the other and supports T cell co-culture. Fold expansion of viable cells after 10-day expansion is depicted.

### Example 9: High Enrichment of Activated CD137⁺ CD154⁺ T Cells after OLP Re-Stimulation

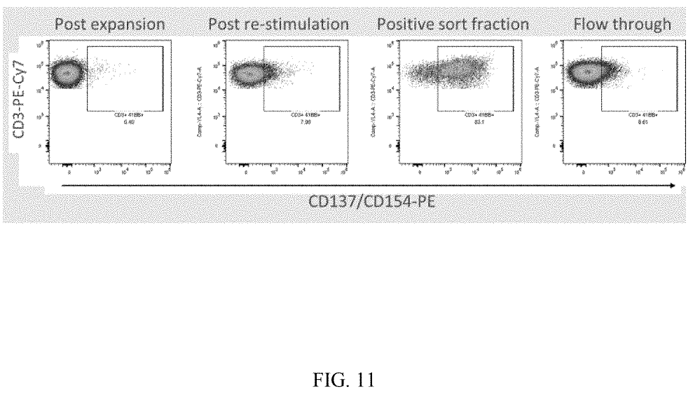

To examine the optimal re-stimulation conditions after the 10-day antigen-specific stimulation and expansion for magnetic bead sorting, optimization studies were performed. Efficient enrichment of activated T cells was achieved after overnight peptide re-stimulation with simultaneous CD137/CD154 sorting. As shown in FIG. 11, the sorted population had a purity of >80% and few activated cells were found in the flow through (negative sort fraction). Sorted CD4⁺ and CD8⁺ T cells were cultured in the presence of MDDCs pulsed with model peptide antigens (of viral origin) for 10 days. T cells were re-stimulated with model peptide antigens on Day 10 for 15 hours, then labeled with CD137/CD154 antibodies and magnetic beads. Labeled cells were separated on magnetic selection columns. Cells were stained for CD3 (T cell marker) and CD137/CD154 (activation markers) to visualize cell populations.

### Example 10: Rapid Non-Specific Stimulation and Expansion of Sorted T Cells

T cells were cultured for 12 days with low-dose IL-2 and either anti-CD3 and CD28 monoclonal antibodies (T Cell TransACT, Miltenyi Biotec) or anti-CD3, CD28, and CD2 monoclonal antibodies (ImmunoCult, StemCell Technologies). Fold expansion and percent viability of the two cultures were determined.

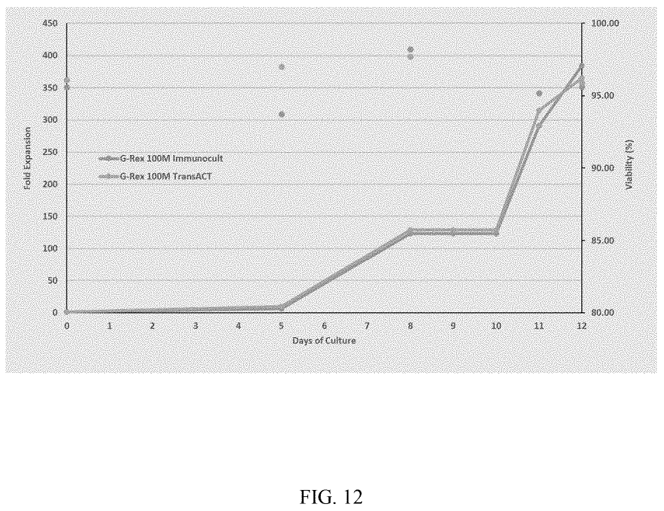

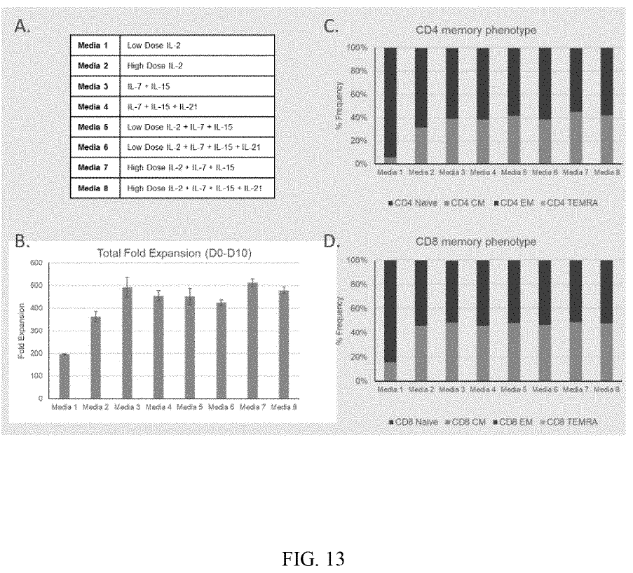

In order to increase the number of antigen-specific T cells to >500 million cells, rapid expansion protocols were tested. T cells were cultured for 12 days with low-dose IL-2 and two different non-specific stimuli: either anti-CD3 and CD28 monoclonal antibodies (T Cell TransACT, Miltenyi Biotec), or anti-CD3, CD28, and CD2 monoclonal antibodies (ImmunoCult, StemCell Technologies) for 12 days. Fold expansion and percent viability of the two cultures were determined. Culturing T cells in GRex 100M flasks generated greater than 350-fold expansion with high (>90%) viability after 12 days (FIG. 12), irrespective of the source of the antibodies or delivery technology (ImmunoCult or T Cell TransACT). The addition of different combinations of cytokines (IL-2, IL-7, IL-15 and IL-21) were also tested (FIG. 13, Panel A) to determine the conditions that result in increased expansion of central memory T cells without inducing increased exhausted or terminal effector memory T cells. Media containing low and high-dose IL-2 alone resulted in significantly lower expansion than the other cytokine-containing media (FIG. 13, Panel B). Low-dose IL-2 resulted in a lower frequency of central memory CD4⁺ and CD8⁺ T cells than the other media (FIG. 13, Panels C and D, respectively, for CD4⁺ and CD8⁺). A central memory phenotype (CM) is identified as CCR7 high and CD45RA low; effector memory (EM) phenotype is CD45RA low and CCR7 low; effector memory re-expressing CD45RA (TEMRA) is CD45RA high and CCR7 negative. Medium containing IL-7/IL-15/IL-21 (Medium 4) yielded the highest mean fold expansions and acceptable memory phenotype and therefore was selected for the rapid expansion protocol yielding exemplary autologous cell therapy compositions (GEN-011).

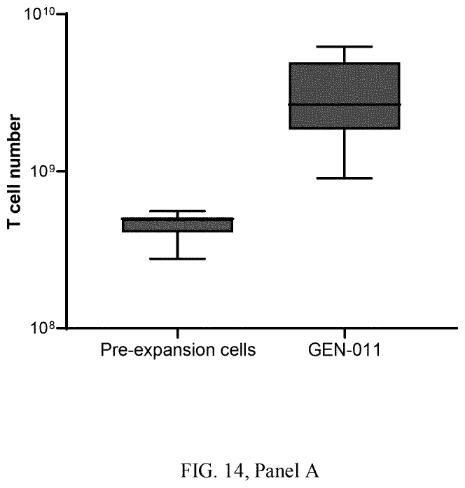

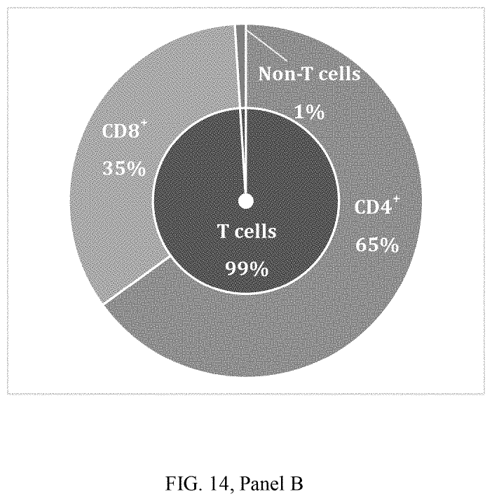

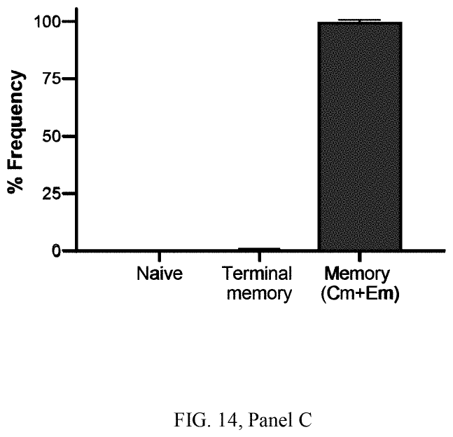

Across multiple development runs initiated with healthy donor material (Example 10), cancer patient material (Example 11), and additional cancer patient material, final expanded autologous adoptive cell therapy compositions comprised an average of 3.3 billion T cells (FIG. 14, Panel A). The exemplary cell therapy compositions were comprised of 99% T cells, 65% of which were CD4⁺ and 35% of which were CD8⁺, and 1% other cell types (FIG. 14, Panel B). Close to 100% of the T cells were of memory phenotype (combined CM and EM phenotypes; FIG. 14, Panel C).

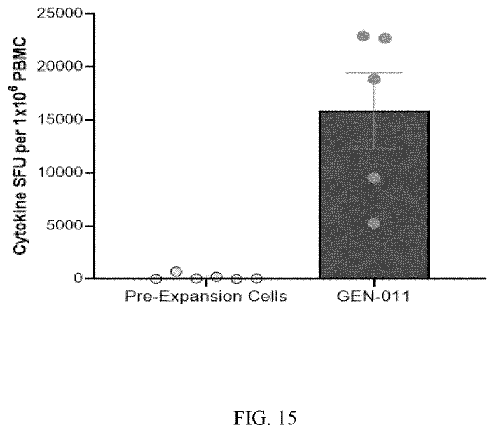

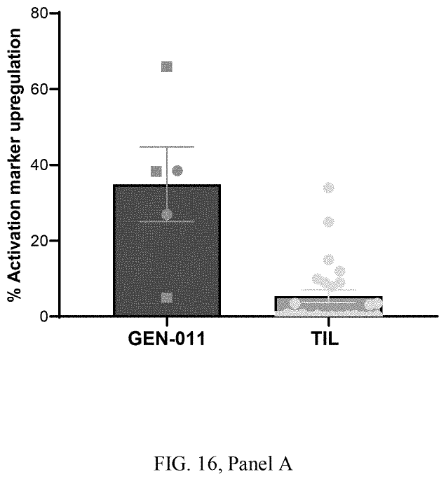

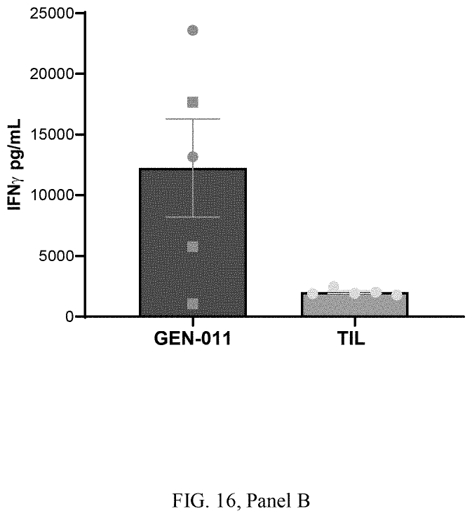

The exemplary autologous adoptive cell therapy compositions were specific for a mean of 89% of the intended antigen targets, i.e., stimulatory tumor antigens for cancer patient material, and model peptide antigens of viral origin for healthy donor material. An average of 16,000 cells per million secreted IFN-gamma and/or TNF-alpha in response to stimulation (dual analyte FluoroSpot assay, FIG. 15). Compared to reported values for Tumor Infiltrating Lymphocyte (TIL) compositions (Chandran S C et al. (2017), Lancet Oncol 18:792-802; Stevanovic S et al. (2015), J Clin Oncol 33:1543-1550; Ritthipichai K et al. (2017), poster, SITC Annual Meeting), T cells of the exemplary cell therapy compositions re-stimulated with specific antigens expressed activation markers (CD154, CD137) and secreted IFN-gamma approximately 6- to 7-fold more than TIL compositions (FIG. 16. Panels A and B, respectively).

### Example 11. In Vitro Characterization of Exemplary Autologous Adoptive Cell Therapy (GEN-011) Derived from an Individual Cancer Patient's T Cells

### Design:

A cancer patient “D” enrolled in Applicant's tumor antigen peptide vaccine clinical trial (NCT03633110) progressed during manufacturing of the investigational vaccine (GEN-009) and was not vaccinated. With the patient's consent, PBMCs and the tumor biopsy that had been collected prior to clinical progression were used for manufacture and non-clinical testing of an exemplary autologous adoptive cell therapy (GEN-011). The tumor was sequenced and screened using the ATLAS method for antigen identification and selection (as described in WO 2018/175505, the contents of which are incorporated herein by reference in their entirety), identifying 28 stimulatory tumor antigens (neoantigens).

The 28 stimulatory tumor antigens were used to make an exemplary autologous adoptive cell therapy according to the methods of Example 4. Briefly, an OLP pool spanning the 28 ATLAS-selected stimulatory antigens was synthesized, with 111 out of 112 peptides successfully manufactured. CD14⁺ monocytes were sorted from the patient's PBMCs and differentiated and matured into MDDCs. The MDDCs were pulsed with the manufactured OLP pool and added to the enriched CD45RO⁺ T cells for a 10-day antigen-specific stimulation and expansion. On Day 10, the antigen-specific stimulated and expanded T cells were re-stimulated with the OLP pool and magnetically sorted based on CD137⁺/CD154⁺ expression. Next, the enriched, antigen-specific re-stimulated T cells were expanded rapidly and non-specifically for 6 days with anti-CD3/CD28 antibodies and cytokines, yielding an exemplary autologous adoptive cell therapy (GEN-011).

### Results:

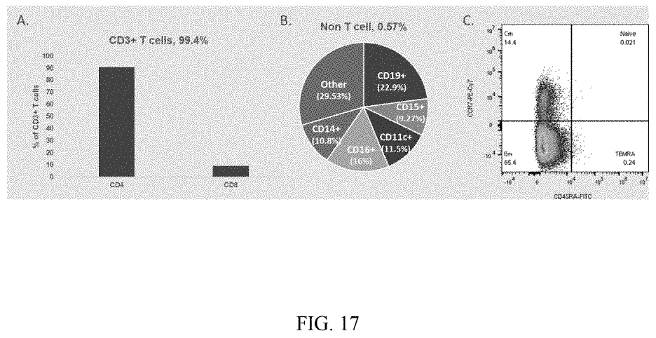

Over 5 billion cells were harvested and cryopreserved, following the final 6-day rapid non-specific expansion step. Flow cytometry for CD3, CD4 and CD8 markers was used to determine the proportions of CD4⁺ and CD8⁺ T cell subsets of the exemplary autologous adoptive cell therapy. The harvested cells were 99.4% T cells (CD3⁺) and >80% viable (FIG. 17, Panel A). The non-T cell population (CD3⁻, <0.6% of the total) was further characterized and comprised a mixture of CD19⁺ B cells, CD16⁺ NK cells, CD14⁺ monocytes, CD11 dendritic cells, and CD15⁺ granulocytes (FIG. 17, Panel B). The memory phenotype of the T cells was characterized by CD45RA and CCR7 expression. A central memory (CM) phenotype is identified as CD45RA low and CCR7 high; effector memory (EM) phenotype is CD45RA low and CCR7 low; effector memory re-expressing CD45RA (TEMRA) phenotype is identified as CD45RA high and CCR7 negative. T cells of the exemplary autologous adoptive cell therapy were predominately of effector memory (85%) and central memory (14%) T cell phenotypes (FIG. 17, Panel C).

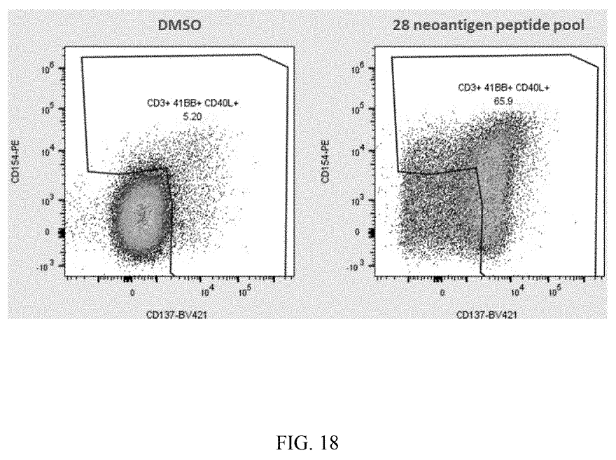

As shown by staining for expression of the activation markers CD154 and CD137, T cells of the exemplary autologous adoptive cell therapy were greater than 65% specific for the OLP pool spanning the 28 ATLAS-selected stimulatory antigens (FIG. 18, Panel B compared to Panel A control T cells stimulated with vehicle control DMSO). In addition, the T cells did not express inhibitory markers such as LAG-3, TIM-3 or PD-1 and proliferated after stimulation with the antigen-specific OLP pool, indicating that the cells are not exhausted and capable of further proliferation.

### Example 12. Killing Effect of Exemplary Autologous Adoptive Cell Therapy (GEN-011) on Tumor Antigen-Expressing Cells

### Design:

Effector T cells from the exemplary autologous adoptive cell therapy (GEN-011) described in Example 11 were co-cultured with target cells comprised of the exemplary patient's autologous antigen presenting cells (APC), which had been isolated from PBMCs and pulsed with either the patient's OLP pool spanning 28 ATLAS-selected stimulatory tumor antigens, or vehicle control (DMSO). Cytotoxicity was quantitated based on luminescence using the Promega CytoTox Glow kit and the formula: % Cytotoxicity=(Luminescence above background)/(Maximal killing lysed control)×100, where background killing was determined in cultures with T cells alone or lymphocyte-depleted PBMCs alone.

### Results:

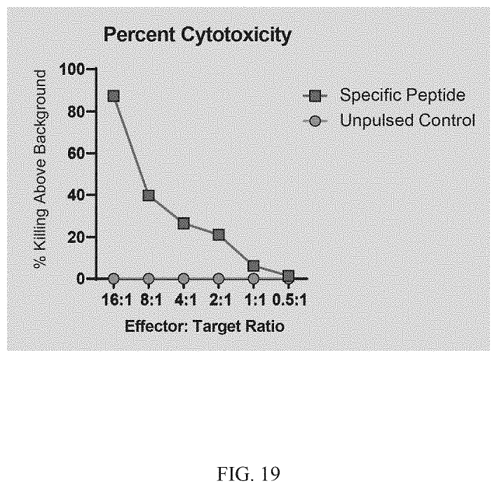

Effector T cells from the exemplary autologous adoptive cell therapy induced dose-dependent killing of the exemplary patient's autologous APC that had been pulsed with the patient's OLP pool spanning 28 ATLAS-selected stimulatory antigens, compared to control APC (FIG. 19). These results show that the exemplary autologous adoptive cell therapy effectively targets and induces T cell-mediated cytotoxicity directed at antigen-expressing cells.

### Example 13. A Phase 1 Study to Evaluate the Safety, Proliferation and Persistence of GEN-011, an Autologous Adoptive Cell Therapy Targeting Tumor Antigens in Solid Tumors

### Objectives:

### Primary Objective:

To evaluate the safety and toxicities of GEN-011 given in 2 dosing regimens.

Secondary Objectives:
To determine the proliferation and persistence of GEN-011 cells in patients using antigen-specific T cell responses and sequencing of the T cell receptor (TCR) beta chain.
To assess clinical response per RECIST and iRECIST criteria, including best response, duration of response, progression free survival (PFS), and survival for those who do not progress up to 2 years post initial dose of GEN-011.

### Exploratory Objectives:

To assess immune cell phenotypes, epitope spreading, and residual or new mutations in residual or recurrent tumor.
To assess immune cell infiltration of the tumor including quantitation, phenotype, and proximity to tumor cells.

### Study Design Overview:

GEN-011 is an investigational, personalized tumor antigen adoptive cell therapy (ACT) that is being developed by Applicant for the treatment of adult patients with solid tumors. A proprietary method developed by Applicant called ATLAS (Antigen Lead Acquisition System) is used to identify true immunogenic tumor antigens from each patient's tumor that are recognized by their own CD4⁺ and/or CD8⁺ T cells. ATLAS-identified tumor antigens are used to stimulate and select autologous T cells derived from peripheral blood mononuclear cells (PBMCs) collected by apheresis to generate an adoptive cell product ex vivo.

This is an open-label, multicenter, first-in-human Phase 1 study of GEN-011 in patients with the following tumor types:
Melanoma
Non-small cell lung cancer (NSCLC)
Squamous cell carcinoma of the head and neck (SCCHN)
Urothelial carcinoma (bladder, ureter, urethra, or renal pelvis)
Renal cell carcinoma (RCC)
Small cell lung cancer (SCLC)
Cutaneous squamous cell carcinoma (CSCC)
Anal squamous cell carcinoma (ASCC)

Patients are enrolled into one of 2 cohorts of 6 DLT-evaluable patients each. One cohort receives a multiple low dose (MLD) regimen of GEN-011 (Schedule 1) to be given without lymphodepletion, and a second cohort receives a single high dose (SHD) regimen of GEN-011 (Schedule 2) after lymphodepletion. All GEN-011 doses are followed by a course of interleukin-2 (IL-2) as costimulatory therapy.

MLD Cohort (Schedule 1):

Patients receive up to 5 intravenous (IV) doses of GEN-011 at 0.2×10⁹ cells given at 4-week intervals until all available cells are depleted (or 5 doses maximum). Each dose of GEN-011 is followed by IL-2 given subcutaneously (SC) at a dose of 125,000 IU/kg/day (with a maximum of 9-10 doses over 2 weeks).

SHD Cohort (Schedule 2):

Patients initially receive a pre-conditioning non-myeloablative lymphodepleting regimen of fludarabine (25 mg/m²) IV and cyclophosphamide (250 mg/m²) IV daily for 3 consecutive days on Days −5, −4, and −3, followed by a single IV dose of GEN-011 on Day 1 at the maximum available cell yield (targeted dose of 1×10⁹ cells; maximum of 3×10⁹ cells). Patients then receive IL-2 given as 6 doses of 600,000 IU/kg IV (daily as tolerated).

Cohort Expansion:

An additional 6 patients are enrolled to either one or both cohorts to confirm the safety, immunologic results, and clinical activity of each regimen.

Each patient's study participation consists of the following periods:

Screening Period:

After providing informed consent, in order to generate the personalized cell product, a tumor sample from the most recent biopsy is collected along with a saliva sample (saliva samples are not collected for SCCHN patients) for exome sequencing to identify the individual tumor-specific mutations. Cells are obtained via leukapheresis for the ATLAS process (to identify and select stimulatory tumor antigens and to subsequently manufacture the patient-specific tumor antigen peptides to be used in the GEN-011 production process) and for cell product manufacture. The entire manufacture process takes approximately 12-15 weeks. During this period, the patient is followed for management of their disease as per standard of care (SOC) and may receive appropriate intervention to manage their disease as indicated. The leukapheresis for product manufacture is performed distant in time from and prior to any immune-suppressive or marrow toxic therapy, such that adequate functional cell numbers are accessible.

Treatment Period:

Six patients are initially enrolled into each cohort. The first 2 patients are enrolled into the MLD Cohort before the SHD Cohort is opened, and dosing is staggered by at least 2 weeks for the first 3 patients enrolled into each cohort. Upon availability of GEN-011 and discontinuation of the patient's SOC treatment, GEN-011 is administered on Day 1 by IV infusion without prophylactic therapy for infusion reactions. If reactions occur, the infusion is stopped, and appropriate symptomatic therapy is given. The GEN-011 infusion is restarted once the reaction is improved to Grade 1 or less. Established management protocols for cytokine release syndrome, neurotoxicity and tumor lysis syndrome are followed. Patients enrolled in the MLD Cohort receive an additional 4 doses of GEN-011 administered 4 weeks apart. Patients enrolled in the SHD Cohort receive a lymphodepletion regimen prior to dosing with GEN-011; both cohorts receive a standard regimen of IL-2 for proliferative stimulation after GEN-011 dosing. Any patients initially assigned to the SHD Cohort whose GEN-011 drug product does not meet the minimum dosing criteria for that cohort is re-assigned to the MLD Cohort. Follow-up evaluations are performed per the Schedule of Assessments, and all patients return at Day 141 for end of treatment (EOT) evaluations.

Post Treatment Period:

Patients return at Days 183, 366 (Week 52), 548 (Week 78), and 732 (Week 104) for follow-up evaluations. All patients who are alive, not lost to follow-up, and/or who have not withdrawn consent are followed for safety and disease outcome for 2 years after their initial dose of GEN-011 (which is also approximately 22 months after the last dose of GEN-011 for the MLD Cohort). Disease assessments and PBMC samples continue to be collected per the Schedule of Assessments until disease progression, initiation of another systemic anticancer therapy, or study closure for a minimum of 2 years.

### Statistical Methods:

Safety Analyses:

No formal statistical analyses are performed on safety data. All recorded AEs are listed and tabulated by system organ class and preferred term. The incidence of AEs is tabulated and reviewed for severity and relationship to GEN-011. Vital signs and clinical laboratory test results are listed and summarized.

Cell Therapy Proliferation/Persistence (CTPP) Analyses:

GEN-011 CTPP analyses are descriptive.

Clinical Activity Analyses:

Clinical activity analyses are descriptive; correlative analyses are descriptive, although statistical tests are used as appropriate to compare changes before and after dosing or between tumor types, but no formal hypothesis testing is planned. Subgroup analysis of various immunologic parameters, as well as rate of response and time to event endpoints, based on demographic and baseline disease characteristics are performed as exploratory analyses, as appropriate.

### Example 14. Polyfunctional Mix of Cells Demonstrated by Single-Cell Cytokine Response Profiles of Exemplary Autologous Adoptive Cell Therapy (GEN-011)

An exemplary autologous adoptive cell therapy (GEN-011) was prepared according to Example 4, using apheresis material from a healthy donor and model peptide antigens (of viral origin). Briefly, antigen-specific T cells were manufactured with the donor's CD4⁺/CD8⁺ cells after presentation of antigens on autologous monocytes derived dendritic cells (MDDCs). After 9 days of antigen-specific cell expansion (37° C. with 5% CO₂), the cells were mixed and divided into two cultures. In one culture, cells were re-stimulated with antigens (2 micromoles/mL final concentration) for 16 hours at 37° C. with 5% CO₂, and incubated with anti-CD154-biotin mAb to capture antigen-specific CD40L-expressing cells. The next day, both CD40L- and 4-1BB-expressing cells were captured by addition of anti-CD154-biotin and anti-CD137-biotin mAbs. In the second culture, cells were re-stimulated with antigens (2 micromoles/mL final concentration) for 16 hours at 37° C. with 5% CO₂. The next day, the cell culture was incubated with anti-CD137-biotin mAb to capture antigen-specific 4-1BB-expressing cells. The antigen-specific cells expressing both CD40L and 4-1BB or only 4-1BB were selected by CliniMACS sort (Miltenyi); positive cells were rapidly expanded for 10 days in the presence of anti-CD3 and -CD28 mAbs and cytokines IL-7, IL-15 and IL-21 in a G-Rex tissue culture vessel. The cell culture supernatant was monitored for glucose and lactate measurement to determine cell growth. Cultures were harvested and GEN-011 drug product was prepared with the addition of cryoformulation buffer and cryostored at <−150° C.

Single-cell secreted cytokine profiles of GEN-011 drug product were analyzed using IsoCode Chips (Isoplexis). The GEN-011 drug product obtained from the second culture (expressing 4-1BB only; designated DEV7) was thawed and re-stimulated by culturing with autologous antigen presenting cells (APCs) and model peptide antigens (of viral origin), anti-CD3 and -CD28 mAbs as positive control, and DMSO as negative control (DMSO). After 20 hrs of co-culture, T cells were enriched and loaded into IsoCode Chips containing 12,000 microchambers pre-patterned with a 32-plex antibody array. Cytokine secretion from 1000-2000 single T cells per sample was detected by a fluorescence ELISA-based assay.

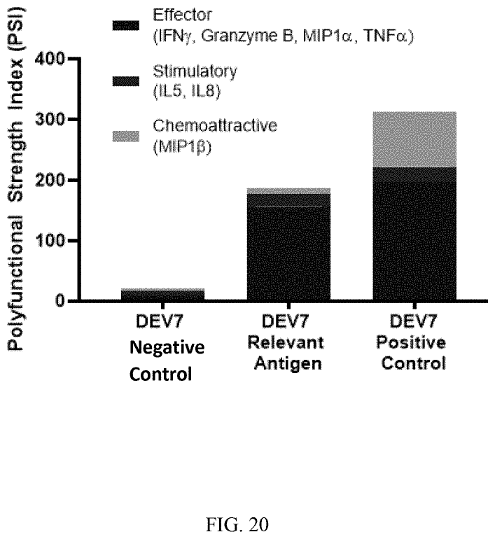

FIG. 20 shows results of single-cell secreted cytokine profiles, grouped according to T cell phenotype for each sample. Effector phenotype cells express IFN-gamma, granzyme B, MIP1-alpha, and TNF-alpha; stimulatory phenotype cells express IL5 and IL8; chemo-attractive phenotype cells express MIP1-beta. Samples are shown on the x axis: DEV7 Relevant Antigen was re-stimulated with the model peptide antigens (of viral origin) used to stimulate the T cells in the first instance, DEV7 Negative Control was re-stimulated with DMSO, and DEV7 Positive Control was re-stimulated with anti-CD3 and -CD28 mAbs. The y axis shows Polyfunctional Strength Index (PSI), defined as the percentage of polyfunctional cells in the sample, multiplied by the intensities of the secreted cytokines. These results show that the exemplary GEN-011 drug product exhibits robust polyfunctional responses.

### Example 15. Enrichment of Specific T Cell Receptors (TCRs) in Exemplary Autologous Adoptive Cell Therapy (GEN-011)

Exemplary autologous adoptive cell therapies (GEN-011) were prepared according to Example 4, using apheresis material from cancer patients and their individual, patient-specific antigens identified by ATLAS screening (drug products denoted DEV1, DEV3, and DEV4) and apheresis material from a healthy donor and model peptide antigens of viral origin (drug product denoted DEV6). Briefly, antigen-specific T cells were manufactured with each donor's CD4⁺/CD8⁺ cells after presentation of antigens on autologous monocytes derived dendritic cells (MDDCs). After 9 days of antigen-specific cell expansion (37° C. with 5% CO₂), the cells were re-stimulated with antigens (2 micromoles/mL final concentration) at 37° C. with 5% CO₂, and incubated with anti-CD154-biotin mAb to capture antigen-specific CD40L-expressing cells. The next day, both CD40L- and 4-1BB-expressing cells were captured by addition of anti-CD154-biotin and anti-CD137-biotin mAbs. Antigen-specific cells expressing both CD40L and 4-1BB were selected by CliniMACS sort (Miltenyi); positive cells were rapidly expanded for 10 days in the presence of anti-CD3 and -CD28 mAbs and cytokines IL-7, IL-15 and IL-21 in a G-Rex tissue culture vessel. The cell culture supernatant was monitored for glucose and lactate measurement to determine cell growth. Cultures were harvested and GEN-011 drug product was prepared with the addition of cryoformulation buffer and cryostored at <−150° C.

For each donor, TCRβ sequencing was performed on PBMCs isolated from apheresis material (pre-expansion), and on exemplary GEN-011 drug product to determine T cell receptor (TCR) diversity. Sequencing was performed by iRepertoire using standard methods.

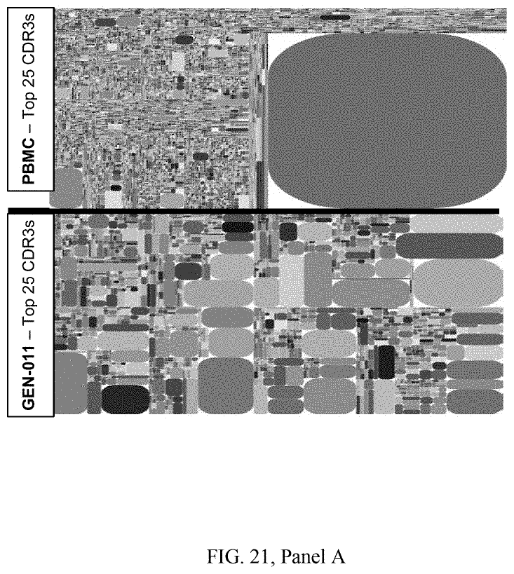

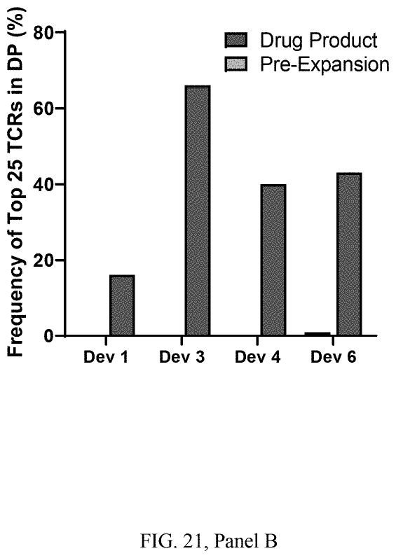

FIG. 21 shows results of TCRβ sequencing. Panel A shows representative tree maps of the top 25 TCRs for each sample; each spot represents a unique V-J-CDR3 and size represents frequency. (NB. Spots are not comparable across squares.) The upper box shows representative results for pre-expansion PBMCs from one cancer patient. The lower box represents the final TCRβ results in the patient's corresponding GEN-011 drug product, DEV4. Comparison of the number of spots and their sizes in the PBMC sample relative to the corresponding GEN-011 drug product demonstrates enrichment of specific TCRs. For DEV4, highly diverse, low abundance TCRs (with a single, strikingly high abundance species) in PBMCs give way to an enriched and balanced mix of less diverse, moderately abundant TCRs. Panel B shows the frequency of the top 25 CDR3s in GEN-011 drug products indicated on the x axis (dark gray bars), plotted relative to their pre-expansion frequencies (light gray bars). DEV1, DEV3, and DEV4 are derived from cancer patients; DEV6 is derived from a healthy donor. The dominant TCRs in GEN-011 drug products are presumed to be antigen-specific.

### Example 16. Characterization of T Cells in Exemplary Autologous Adoptive Cell Therapy Using Freshly-Prepared or Cryopreserved MDDCs

Exemplary autologous adoptive cell therapy compositions were prepared according to Example 4, using apheresis material from a healthy donor and model peptide antigens (derived from Influenza A nucleoprotein) in order to compare antigen-specific T cell expansion methods based on freshly-prepared and cryopreserved mature MDDCs.

Briefly, CD14⁺ monocytes were isolated from apheresis material using CD14 microbeads and an automated cell separation instrument (CliniMACS Plus, or equivalent instrument). CD4⁺ and CD8⁺ T cells were isolated from the cell population using CD4/CD8 microbeads (Miltenyi Biotec). The CD14⁺ monocytes were differentiated and matured in vitro into MDDCs using the ImmunoCult Dendritic Cell Culture Kit (StemCell Technologies). Mature MDDCs were cryopreserved using Sepax C Pro and CryoMed at 2.5×10⁶ cells per ml, and maintained for 3 weeks at −150° C.

Next, the isolated CD4⁺ and CD8⁺ T cells were antigen-specifically expanded in the presence of either the thawed, cryopreserved MDDCs or in the presence of freshly-prepared MDDCs, and in each case pulsed with the model overlapping peptide antigens. After 10 days of co-culture (at 37° C. with 5% CO₂), the cells were re-stimulated with the model peptide antigens and magnetically sorted based on CD137⁺ expression. The antigen-specific cells expressing CD137 were then rapidly and non-specifically expanded for 10 days in the presence of anti-CD3 and -CD28 mAbs and cytokines IL-7, IL-15 and IL-21, yielding exemplary autologous adoptive cell therapy compositions.

The cell culture supernatants were monitored for glucose and lactate to determine cell growth during both the antigen-specific expansion and the rapid expansion phases. Cell viability and fold-expansion were also measured during both the antigen-specific expansion and the rapid expansion phases.

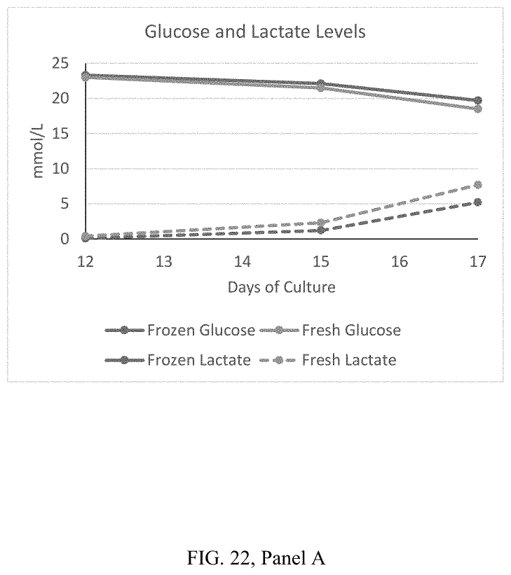

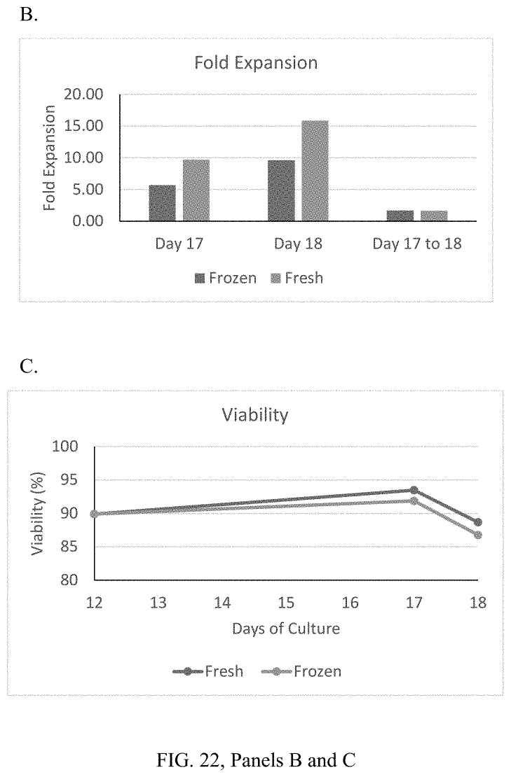

FIG. 22 shows T cell characteristics of exemplary autologous adoptive cell therapy compositions at the antigen-specific expansion phase, during which T cells were cultured in the presence of either fresh MDDCs (Fresh) or cryopreserved MDDCs (Frozen) pulsed with model overlapping peptide antigens. Panel A shows glucose levels (solid lines, indicating mmol/L of supernatant in the Fresh and Frozen arms of the study) and lactate levels (dashed lines, indicating mmol/L of supernatant in the Fresh and Frozen arms) expressed by antigen-specifically expanded T cells over days 12 to 17 in culture. Panel B shows the fold-expansion of T cells in response to specific antigens in the Fresh and Frozen arms, on days 17 and 18 in culture. Panel C shows the percent viability of antigen-specifically expanded T cells in the Fresh and Frozen arms, from days 12 to 18 in culture.

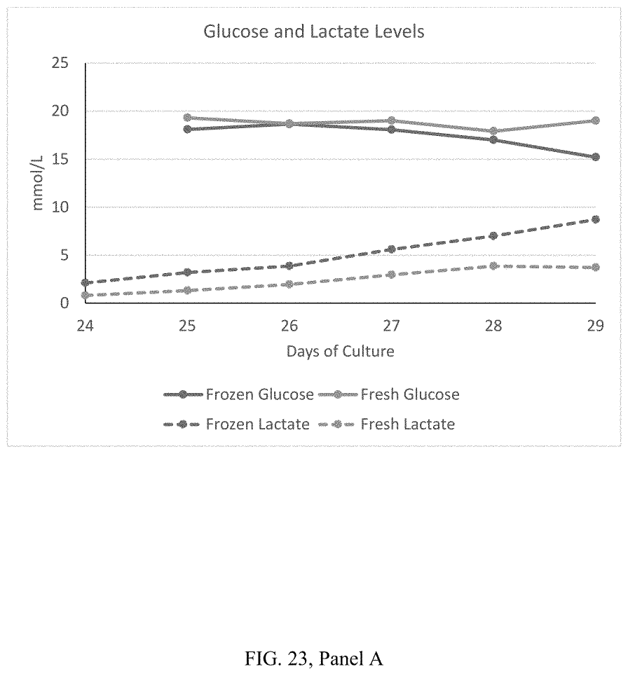

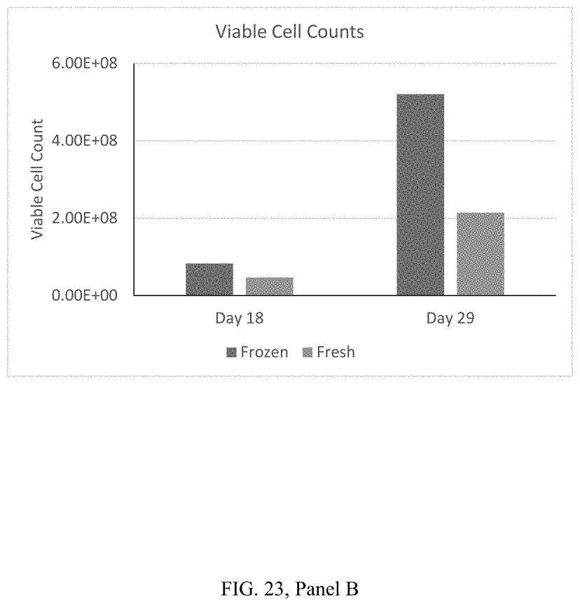

FIG. 23 shows T cell characteristics of exemplary autologous adoptive cell therapy compositions at the rapid, non-specific expansion phase, following the antigen-specific expansion phase shown in FIG. 22. Panel A shows glucose levels (solid lines; indicating mmol/L of supernatant in the Fresh and Frozen arms of the study) and lactate levels (dashed lines, indicating mmol/L of supernatant in the Fresh and Frozen arms) expressed by non-specifically expanded T cells over days 24 to 29 in culture. Panel B shows the percent viability of non-specifically expanded T cells in the Fresh and Frozen arms on days 18 and 29 in culture.

These results indicate that antigen-specific expansion of sorted T cells may be performed using either freshly-prepared or cryopreserved mature MDDCs.

LISTING OF SEQUENCES

Heparanase isoform 1, preproprotein, NP_001092010.1, NP_006656.2 (SEQ ID NO:
6)

| | |
|---|---|
| 1 | mllrskpalp pplmllllgp lgplspgalp rpaqaqdvvd ldfftqeplh lvspsflsvt |
| 61 | idanlatdpr flillgspkl rtlarglspa ylrfggtktd flifdpkkes tfeersywqs |
| 121 | qvnqdickyg sippdveekl rlewpyqeql llrehyqkkf knstysrssv dvlytfancs |
| 181 | gldlifglna llrtadlqwn ssnaqllldy csskgynisw elgnepnsfl kkadifings |
| 241 | qlgedfiqlh kllrkstfkn aklygpdvgq prrktakmlk sflkaggevi dsvtwhhyyl |
| 301 | ngrtatkedf lnpdvldifi ssvqkvfqvv estrpgkkvw lgetssaygg gapllsdtfa |
| 361 | agfmwldklg lsarmgievv mrqvffgagn yhlvdenfdp lpdywlsllf kklvgtkvlm |
| 421 | asvqgskrrk lrvylhctnt dnprykegdl tlyainlhnv tkylrlpypf snkqvdkyll |
| 481 | rplgphglls ksvqlngltl kmvddqtlpp lmekplrpgs slglpafsys ffvirnakva |
| 541 | aci |

Heparanase isoform 2, preproprotein, NP_001159970.1 (SEQ ID NO: 7)

| | |
|---|---|
| 1 | mllrskpalp pplmllllgp lgplspgalp rpaqaqdvvd ldfftqeplh lvspsflsvt |
| 61 | idanlatdpr flillgspkl rtlarglspa ylrfggtktd flifdpkkes tfeersywqs |
| 121 | qvngdickyg sippdveekl rlewpyqeql llrehyqkkf knstysrssv dvlytfancs |
| 181 | gldlifglna llrtadlqwn ssnaqllldy csskgynisw elgnepnsfl kkadifings |
| 241 | qlgedfiqlh kllrkstfkn aklygpdvgq prrktakmlk sflkaggevi dsvtwhhyyl |
| 301 | ngrtatkedf lnpdvldifi ssvqkvfqdy wlsllfkklv gtkvlmasvq gskrrklrvy |
| 361 | lhctntdnpr ykegdltlya inlhnvtkyl rlpypfsnkq vdkyllrplg phgllsksvq |
| 421 | lngltlkmvd dqtlpplmek plrpgsslgl pafsysffvi rnakvaaci |

SMAD family member 4 , mothers against decapentaplegic homolog 4, NP_005350.1
(SEQ ID NO: 8)

| | |
|---|---|
| 1 | mdnmsitntp tsndaclsiv hslmchrqgg esetfakrai eslvkklkek kdeldslita |
| 61 | ittngahpsk cvtiqrtldg rlqvagrkgf phviyarlwr wpdlhknelk hvkycqyafd |
| 121 | lkcdsvcvnp yhyervvspg idlsgltlqs napssmmvkd eyvhdfegqp slsteghsiq |
| 181 | tiqhppsnra stetystpal lapsesnats tanfpnipva stsqpasilg gshsegllqi |
| 241 | asgpqpgqqq ngftgqpaty hhnstttwtg srtapytpnl phhqnghlqh hppmpphpgh |
| 301 | ywpvhnelaf qppisnhpap eywcsiayfe mdvqvgetfk vpsscpivtv dgyvdpsggd |
| 361 | rfclgqlsnv hrteaierar lhigkgvqle ckgegdvwvr clsdhavfvq syyldreagr |
| 421 | apgdavhkiy psayikvfdl rqchrqmqqg aataqaaaaa qaaavagnip gpgsvggiap |
| 481 | aislsaaagi gvddlrrlci lrmsfvkgwg pdyprqsike tpcwieihlh ralqlldevl |
| 541 | htmpiadpqp ld |

Cadherin 3, isoform 1 preproprotein, NP_001784.2 (SEQ ID NO: 9)

| | |
|---|---|
| 1 | mglprgplas llllqvcwlq caaseperav freaevtlea ggaeqepgqa lgkvfmgcpg |
| 61 | qepalfstdn ddftvrnget vqerrslker nplkifpskr ilrrhkrdwv vapisvpeng |
| 121 | kgpfpqrlnq lksnkdrdtk ifysitgpga dsppegvfav eketgwllln kpldreeiak |
| 181 | yelfghavse ngasvedpmn isiivtdqnd hkpkftqdtf rgsvlegvlp gtsvmqvtat |
| 241 | deddaiytyn gvvaysihsq epkdphdlmf tihrstgtis vissgldrek vpeytltiqa |
| 301 | tdmdgdgstt tavavveild andnapmfdp qkyeahvpen avghevqrlt vtdldapnsp |
| 361 | awratylimg gddgdhftit thpesnqgil ttrkgldfea knqhtlyvev tneapfvlkl |
| 421 | ptstativvh vedvneapvf vppskvvevq egiptgepvc vytaedpdke nqkisyrilr |
| 481 | dpagwlamdp dsgqvtavgt ldredeqfvr nniyevmvla mdngsppttg tgtllltlid |
| 541 | vndhgpvpep rqiticnqsp vrqvlnitdk dlsphtspfq aqltddsdiy wtaevneegd |
| 601 | tvvlslkkfl kqdtydvhls lsdhgnkeql tviratvcdc hghvetcpgp wkggfilpvl |
| 661 | gavlallfll lvllllvrkk rkikeplllp eddtrdnvfy ygeegggeed qdyditqlhr |
| 721 | glearpevvl rndvaptiip tpmyrprpan pdeignfiie nlkaantdpt appydtllvf |
| 781 | dyegsgsdaa slssltssas dqdqdydyln ewgsrfkkla dmygggedd |

Cadherin 3, isoform 2 precursor, NP_001304124.1 (SEQ ID NO: 10)

| | |
|---|---|
| 1 | mglprgplas llllqvcwlq caaseperav freaevtlea ggaeqepgqa lgkvfmgcpg |
| 61 | qepalfstdn ddftvrnget vqerrslker nplkifpskr ilrrhkrdwv vapisvpeng |
| 121 | kgpfpqrlnq lksnkdrdtk ifysitgpga dsppegvfav eketgwllln kpldreeiak |
| 181 | yelfghavse ngasvedpmn isiivtdqnd hkpkftqdtf rgsvlegvlp gtsvmqvtat |
| 241 | deddaiytyn gvvaysihsq epkdphdlmf tihrstgtis vissgldrek vpeytltiqa |
| 301 | tdmdgdgstt tavavveild andnapmfdp qkyeahvpen avghevqrlt vtdldapnsp |
| 361 | awratylimg gddgdhftit thpesnqgil ttrkgldfea knqhtlyvev tneapfvlkl |
| 421 | ptstativvh vedvneapvf vppskvvevq egiptgepvc vytaedpdke nqkisyrilr |
| 481 | dpagwlamdp dsgqvtavgt ldredeqfvr nniyevmvla mdngsppttg tgtllltlid |
| 541 | vndhgpvpep rgiticnqsp vrqvlnitdk dlsphtspfq aqltddsdiy wtaevneegd |
| 601 | tvvlslkkfl kqdtydvhls lsdhgnkeql tviratvcdc hghvetcpgp wkggfilpvl |
| 661 | gavlallfll lvllllvrkk rkikeplllp eddtrdnvfy ygeegggeed qdyditqlhr |
| 721 | glearpevvl rndvaptiip tpmyrprpan pdeignfiie grgergsqrg ngglqlargr |
| 781 | trrs |

Cadherin 3, isoform 3, NP_001304125.1 (SEQ ID NO: 11)

| | |
|---|---|
| 1 | mgcpgqepal fstdnddftv rngetvqerr slkernplki fpskrilrrh krdwvvapis |
| 61 | vpengkgpfp qrlnqlksnk drdtkifysi tgpgadsppe gvfaveketg wlllnkpldr |
| 121 | eeiakyelfg haysengasv edpmnisiiv tdqndhkpkf tqdtfrgsvl egvlpgtsvm |
| 181 | qvtatdedda iytyngvvay sihsqepkdp hdlmftihrs tgtisvissg ldrekvpeyt |
| 241 | ltiqatdmdg dgstttavav veildandna pmfdpqkyea hvpenavghe vqrltvtdld |
| 301 | apnspawrat ylimggddgd hftitthpes nqgilttrkg ldfeaknqht lyvevtneap |
| 361 | fvlklptsta tivvhvedvn eapvfvppsk vvevqegipt gepvcvytae dpdkenqkis |
| 421 | yrilrdpagw lamdpdsgqv tavgtldred eqfvrnniye vmvlamdngs ppttgtgtll |
| 481 | ltlidvndhg pvpeprqiti cnqspvrqvl nitdkdlsph tspfqaqltd dsdiywtaev |
| 541 | neegdtvvls lkkflkqdty dvhlslsdhg nkeqltvira tvcdchghve tcpgpwkggf |
| 601 | ilpvlgavla llflllvlll lvrkkrkike plllpeddtr dnvfyygeeg ggeedqdydi |
| 661 | tqlhrglear pevvlrndva ptiiptpmyr prpanpdeig nfiienlkaa ntdptappyd |
| 721 | tllvfdyegs gsdaaslssl tssasdqdqd ydylnewgsr fkkladmygg gedd |

Chorionic gonadotropin beta subunit 3, precursor, NP_000728.1 (SEQ ID NO: 12)

| | |
|---|---|
| 1 | memfqgllll lllsmggtwa skeplrprcr pinatlavek egcpvqtvn tticagycpt |
| 61 | mtrvlqgvlp alpqvvcnyr dvrfesirlp gcprgvnpvv syavalscqc alcrrsttdc |
| 121 | ggpkdhpltc ddprfqdsss skapppslps psrlpgpsdt pilpq |

Chorionic gonadotropin beta subunit 5, precursor, NP_149032.1 (SEQ ID NO: 13)

| | |
|---|---|
| 1 | memfqgllll lllsmggtwa skeplrprcr pinatlavek egcpvcitvn tticagycpt |
| 61 | mtrvlqgvlp alpqvvcnyr dvrfesirlp gcprgvnpvv syavalscqc alcrrsttdc |
| 121 | ggpkdhpltc ddprfqdsss skapppslps psrlpgpsdt pilpq |

Cytochrome c oxidase assembly factor 1 homolog, isoform a, NP_001308126.1,
NP_001308127.1, NP_001308128.1, NP_001308129.1, NP_001337853.1,
NP_001337854.1, NP_001337855.1, NP_001337856.1, NP_060694.2 (SEQ ID NO: 14)

| | |
|---|---|
| 1 | mmwqkyagsr rsmplgaril fhgvfyaggf aivyyliqkf hsralyykla veqlqshpea |
| 61 | qealgppini hylklidren fvdivdaklk ipvsgskseg llyvhssrgg pfqrwhldev |
| 121 | flelkdgqqi pvfklsgeng devkke |

Cytochrome c oxidase assembly factor 1 homolog, isoform b, NP_001308130.1
(SEQ ID NO: 15)

| | |
|---|---|
| 1 | mplgarilfh gvfyaggfai vyyliqkfhs ralyyklave qlqshpeaqe algppinihy |
| 61 | lklidrenfv divdaklkip vsgsksegll yvhssrggpf qrwhldevfl elkdgqqipv |
| 121 | fklsgengde vkke |

Cytochrome c oxidase assembly factor 1 homolog, isoform c, NP_001308131.1,
NP_001308132.1, NP_001308133.1, NP_001308134.1 (SEQ ID NO: 16)

| | |
|---|---|
| 1 | mmwqkyagsr rsmplgaril fhgvfyaggf aivyyliqsk ypasrlrpdl llacscssir |
| 61 | gnt |

Cytochrome c oxidase assembly factor 1 homolog, isoform d, NP_001337857.1
(SEQ ID NO: 17)

| | |
|---|---|
| 1 | mqeaggqclw eqgsfstvcs mpgalplcit sfkfhsraly yklaveqlqs hpeaqealgp |
| 61 | pinihylkli drenfvdivd aklkipvsgs ksegllyvhs srggpfqrwh ldevflelkd |
| 121 | gqqipvfkls gengdevkke |

Estrogen receptor binding site associated, antigen, 9, NP_001265867.1,
NP_004206.1, NP_936056.1, NP_001308129.1, (SEQ ID NO: 18)

| | |
|---|---|
| 1 | maitqfrlfk fctclatvfs flkrlicrsg rgrklsgdqi tlpttvdyss vpkqtdveew |
| 61 | tswdedapts vkieggngnv atqqnsleql epdyfkdmtp tirktqkivi kkreplnfgi |
| 121 | pdgstgfssr laatqdlpfi hqsselgdld twqentnawe eeedaawqae evlrqqklad |
| 181 | rekraaeqqr kkmekeaqrl mkkeqnkigv kls |

ETS transcription factor, isoform a, NP_001964.2 (SEQ ID NO: 19)

| | |
|---|---|
| 1 | mdsaitlwqf llqllqkpqn khmicwtsnd gqfkllqaee varlwgirkn kpnmnydkls |
| 61 | ralryyyvkn iikkvngqkf vykfvsypei lnmdpmtvgr iegdceslnf sevsssskdv |
| 121 | enggkdkppq pgaktssrnd yihsglyssf tlnslnssnv klfklikten paeklaekks |
| 181 | pqeptpsvik fvttpskkpp vepvaatisi gpsispssee tiqaletivs pklpsleapt |
| 241 | sasnvmtafa ttppissipp lqepprtpsp plsshpdidt didsvasqpm elpenlslep |
| 301 | kdqdsvllek dkvnnssrsk kpkglelapt lvitssdpsp lgilspslpt asltpaffsq |
| 361 | tpiiltpspl lssihfwstl spvaplspar lqgantlfqf psvinshgpf tlsgldgpst |
| 421 | pgpfspdlqk t |

ETS transcription factor, isoform b, NP_068567.1 (SEQ ID NO: 20)

| | |
|---|---|
| 1 | mdsaitlwqf llqllqkpqn khmicwtsnd gqfkllqaee varlwgirkn kpnmnydkls |
| 61 | ralryyyvkn iikkvngqkf vykfvsypei lnmdpmtvgr iegdceslnf sevsssskdv |
| 121 | enggkdkppq pgaktssrnd yihsglyssf tlnslnssnv klfklikten paeklaekks |
| 181 | pqeptpsvik fvttpskkpp vepvaatisi gpsispssee tiqaletivs pklpsleapt |
| 241 | sasnvmtafa ttppissipp lqepprtpsp plsshpdidt didsvasqpm elpenlslep |
| 301 | kdqdsvllek dkvnnssrsk kpkglelapt lvitssdpsp lgilspslpt asltpaffsq |
| 361 | vacslfmvsp llsficpfkq iqnlytqvcf lllrfvlerl cvtvm |

Receptor tyrosine-protein kinase erbB-2, isoform a precursor, NP_004439.2
(SEQ ID NO: 21)

| | |
|---|---|
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dpinnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvqlvtql mpygclldhv renrgrlgsq dllnwcmqia kgmsyledvr |
| 841 | lvhrdlaarn vlvkspnhvk itdfglarll dideteyhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedlgpaspl dstfyrslle dddmgdlvda |
| 1021 | eeylvpqqgf fcpdpapgag gmvhhrhrss strsgggdlt lglepseeea prsplapseg |
| 1081 | agsdvfdgdl gmgaakglqs lpthdpsplq rysedptvpl psetdgyvap ltcspqpeyv |
| 1141 | nqpdvrpqpp spregplpaa rpagatlerp ktlspgkngv vkdvfafgga venpeyltpq |
| 1201 | ggaapqphpp pafspafdnl yywdqdpper gappstfkgt ptaenpeylg ldvpv |

Receptor tyrosine-protein kinase erbB-2, isoform b, NP_001005862.1 (SEQ ID
NO: 22)

| | |
|---|---|
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctivcplhnq evtaedgtqr |
| 301 | cekcskpcar vcyglgmehl revravtsan iqefagckki fgslaflpes fdgdpasnta |
| 361 | plqpeqlqvf etleeitgyl yisawpdslp dlsvfqnlqv irgrilhnga ysltlqglgi |
| 421 | swlglrslre lgsglalihh nthlcfvhtv pwdqlfrnph qallhtanrp edecvgegla |
| 481 | chqlcarghc wgpgptqcvn csqflrgqec veecrvlqgl preyvnarhc lpchpecqpq |
| 541 | ngsvtcfgpe adqcvacahy kdppfcvarc psgvkpdlsy mpiwkfpdee gacqpcpinc |
| 601 | thscvdlddk gcpaeqrasp ltsiisavvg illvvvlgvv fgilikrrqq kirkytmrrl |
| 661 | lqetelvepl tpsgampnqa qmrilketel rkvkvlgsga fgtvykgiwi pdgenvkipv |
| 721 | aikvlrents pkankeilde ayvmagvgsp yvsrllgicl tstvqlvtql mpygclldhv |
| 781 | renrgrlgsq dllnwcmqia kgmsyledvr lvhrdlaarn vlvkspnhvk itdfglarll |
| 841 | dideteyhad ggkvpikwma lesilrrrft hqsdvwsygv tvwelmtfga kpydgipare |
| 901 | ipdllekger lpqppictid vymimvkcwm idsecrprfr elvsefsrma rdpqrfvviq |
| 961 | nedlgpaspl dstfyrslle dddmgdlvda eeylvpqqgf fcpdpapgag gmvhhrhrss |
| 1021 | strsgggdlt lglepseeea prsplapseg agsdvfdgdl gmgaakglqs lpthdpsplq |
| 1081 | rysedptvpl psetdgyvap ltcspqpeyv nqpdvrpqpp spregplpaa rpagatlerp |
| 1141 | ktlspgkngv vkdvfafgga venpeyltpq ggaapqphpp pafspafdnl yywdqdpper |
| 1201 | gappstfkgt ptaenpeylg ldvpv |

Receptor tyrosine-protein kinase erbB-2, isoform c, NP_001276865.1 (SEQ ID
NO: 23)

| | |
|---|---|
| 1 | mprgswkpqv ctgtdmklrl paspethldm lrhlyqgcqv vqgnleltyl ptnaslsflq |
| 61 | diqevqgyvl iahnqvrqvp lqrlrivrgt qlfednyala vldngdpinn ttpvtgaspg |
| 121 | glrelqlrsl teilkggvli qrnpqlcyqd tilwkdifhk nnqlaltlid tnrsrachpc |
| 181 | spmckgsrcw gessedcqsl trtvcaggca rckgplptdc cheqcaagct gpkhsdclac |
| 241 | lhfnhsgice lhcpalvtyn tdtfesmpnp egrytfgasc vtacpynyls tdvgsctlvc |
| 301 | plhnqevtae dgtqrcekcs kpcarvcygl gmehlrevra vtsaniqefa gckkifgsla |
| 361 | flpesfdgdp asntaplqpe qlqvfetlee itgylyisaw pdslpdlsvf qnlqvirgri |
| 421 | lhngaysltl qglgiswlgl rslrelgsgl alihhnthlc fvhtvpwdql frnphqallh |
| 481 | tanrpedecv geglachqlc arghcwgpgp tqcvncsqfl rgqecveecr vlqglpreyv |
| 541 | narhclpchp ecqpqngsvt cfgpeadqcv acahykdppf cvarcpsgvk pdlsympiwk |
| 601 | fpdeegacqp cpincthscv dlddkgcpae qraspltsii savvgillvv vlgvvfgili |
| 661 | krrqqkirky tmrrllqete lvepltpsga mpnqaqmril ketelrkvkv lgsgafgtvy |
| 721 | kgiwipdgen vkipvaikvl rentspkank eildeayvma gvgspyvsrl lgicltstvq |
| 781 | lvtqlmpygc lldhvrenrg rlgsqdllnw cmqiakgmsy ledvrlvhrd laarnvlvks |
| 841 | pnhvkitdfg larlldidet eyhadggkvp ikwmalesil rrrfthqsdv wsygvtvwel |
| 901 | mtfgakpydg ipareipdll ekgerlpqpp ictidvymim vkcwmidsec rprfrelvse |
| 961 | fsrmardpqr fvviqnedlg paspldstfy rslledddmg dlvdaeeylv pqqgffcpdp |
| 1021 | apgaggmvhh rhrssstrsg ggdltlglep seeeaprspl apsegagsdv fdgdlgmgaa |
| 1081 | kglqslpthd psplqrysed ptvplpsetd gyvapltcsp qpeyvnqpdv rpqppspreg |
| 1141 | plpaarpaga tlerpktlsp gkngvvkdvf afggavenpe yltpqggaap qphpppafsp |
| 1201 | afdnlyywdq dppergapps tfkgtptaen peylgldvpv |

Receptor tyrosine-protein kinase erbB-2, isoform d precursor, NP_001276866.1
(SEQ ID NO: 24)

| | |
|---|---|
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvqlvtql mpygclldhv renrgrlgsq dllnwcmqia kgmsyledvr |
| 841 | lvhrdlaarn vlvkspnhvk itdfglarll dideteyhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedlgpaspl dstfyrslle dddmgdlvda |
| 1021 | eeylvpqqgf fcpdpapgag gmvhhrhrss strnm |

Receptor tyrosine-protein kinase erbB-2, isoform e, NP_001276867.1 (SEQ ID
NO: 25)

| | |
|---|---|
| 1 | mklrlpaspe thldmlrhly qgcqvvqgnl eltylptnas lsflqdiqev qgyvliahnq |
| 61 | vrqvplqrlr ivrgtqlfed nyalavldng dpinnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctlvcplhnq evtaedgtqr |
| 301 | cekcskpcar vcyglgmehl revravtsan iqefagckki fgslaflpes fdgdpasnta |
| 361 | plqpeqlqvf etleeitgyl yisawpdslp dlsvfqnlqv irgrilhnga ysltlqglgi |
| 421 | swlglrslre lgsglalihh nthlcfvhtv pwdqlfrnph qallhtanrp edecvgegla |
| 481 | chqlcarghc wgpgptqcvn csqflrgqec veecrvlqgl preyvnarhc lpchpecqpq |
| 541 | ngsvtcfgpe adqcvacahy kdppfcvarc psgvkpdlsy mpiwkfpdee gacqpcpinc |
| 601 | ths |

Inosine monophosphate dehydrogenase 2 , NP_000875.2 (SEQ ID NO: 26)

| | |
|---|---|
| 1 | madylisggt syvpddglta qqlfncgdgl tyndflilpg yidftadqvd ltsaltkkit |
| 61 | lktplvsspm dtvteagmai amaltggigf ihhnctpefq anevrkvkky eqgfitdpvv |
| 121 | lspkdrvrdv feakarhgfc gipitdtgrm gsrlvgiiss rdidflkeee hdcfleeimt |
| 181 | kredlvvapa gitlkeanei lqrskkgklp ivneddelva iiartdlkkn rdyplaskda |
| 241 | kkqllcgaai gtheddkyrl dllaqagvdv vvldssqgns ifqinmikyi kdkypnlqvi |
| 301 | ggnvvtaaqa knlidagvda lrvgmgsgsi citqevlacg rpqatavykv seyarrfgvp |
| 361 | viadggiqnv ghiakalalg astvmmgsll aatteapgey ffsdgirlkk yrgmgsldam |
| 421 | dkhlssqnry fseadkikva qgvsgavqdk gsihkfvpyl iagiqhscqd igaksltqvr |
| 481 | ammysgelkf ekrtssaqve ggvhslhsye krlf |

KRAS proto-oncogene, GTPase, isoform a, NP_203524.1 (SEQ ID NO: 27)

| | |
|---|---|
| 1 | mteyklvvvg aggvgksalt iqliqnhfvd eydptiedsy rkqvvidget clldildtag |
| 61 | qeeysamrdq ymrtgegflc vfainntksf edihhyreqi krvkdsedvp mv1vgnkcdl |
| 121 | psrtvdtkqa qdlarsygip fietsaktrq rvedafytiv reirqyrlkk iskeektpgc |
| 181 | vkikkciim |

KRAS proto-oncogene, GTPase, isoform b, NP_004976.2 (SEQ ID NO: 28)

| | |
|---|---|
| 1 | mteyklvvvg aggvgksalt iqliqnhfvd eydptiedsy rkqvvidget clldildtag |
| 61 | qeeysamrdq ymrtgegflc vfainntksf edihhyreqi krvkdsedvp mvlvgnkcdl |
| 121 | psrtvdtkqa qdlarsygip fietsaktrq gvddafytiv reirkhkekm skdgkkkkkk |
| 181 | sktkcvim |

Transforming growth factor beta receptor 2, isoform A precursor,
NP_001020018.1 (SEQ ID NO: 29)

| | |
|---|---|
| 1 | mgrgllrglw plhivlwtri astipphvqk sdvemeaqkd eiicpscnrt ahplrhinnd |
| 61 | mivtdnngav kfpqlckfcd vrfstcdnqk scmsncsits icekpqevcv avwrkndeni |
| 121 | tletvchdpk lpyhdfiled aaspkcimke kkkpgetffm cscssdecnd niifseeynt |
| 181 | snpdlllvif qvtgisllpp lgvaisviii fycyrvnrqq klsstwetgk trklmefseh |
| 241 | caiileddrs disstcanni nhntellpie ldtlvgkgrf aevykaklkq ntseqfetva |
| 301 | vkifpyeeya swktekdifs dinlkhenil qfltaeerkt elgkqywlit afhakgnlqe |
| 361 | yltrhviswe dlrklgssla rgiahlhsdh tpcgrpkmpi vhrdlkssni lvkndltccl |
| 421 | cdfglslrld ptlsvddlan sgqvgtarym apevlesrmn lenvesfkqt dvysmalvlw |
| 481 | emtsrcnavg evkdyeppfg skvrehpcve smkdnvlrdr grpeipsfwl nhqgiqmvce |
| 541 | tltecwdhdp earltaqcva erfselehld rlsgrscsee kipedgslnt tk |

Transforming growth factor beta receptor 2, isoform B precursor, NP_003233.4
(SEQ ID NO: 30)

| | |
|---|---|
| 1 | mgrgllrglw plhivlwtri astipphvqk svnndmivtd nngavkfpql ckfcdvrfst |
| 61 | cdnqkscmsn csitsicekp qevcvavwrk ndenitletv chdpklpyhd filedaaspk |
| 121 | cimkekkkpg etffmcscss decndniifs eeyntsnpdl llvifqvtgi sllpplgvai |
| 181 | sviiifycyr vnrqqklsst wetgktrklm efsehcaiil eddrsdisst canninhnte |
| 241 | llpieldtlv gkgrfaevyk aklkqntseq fetvavkifp yeeyaswkte kdifsdinlk |
| 301 | henilqflta eerktelgkq ywlitafhak gnlqeyltrh viswedlrkl gsslargiah |
| 361 | lhsdhtpcgr pkmpivhrdl kssnilvknd ltcclcdfgl slrldptlsv ddlansgqvg |
| 421 | tarymapevl esrmnlenve sfkqtdvysm alvlwemtsr cnavgevkdy eppfgskvre |
| 481 | hpcvesmkdn vlrdrgrpei psfwlnhqgi qmvcetltec wdhdpearlt aqcvaerfse |
| 541 | lehldrlsgr scseekiped gslnttk |

Actinin alpha 4, isoform 1, NP_004915.2 (SEQ ID NO: 31)

| | |
|---|---|
| 1 | mvdyhaanqs yqygpssagn gaggggsmgd ymaqeddwdr dllldpawek qqrktftawc |
| 61 | nshlrkagtq ienidedfrd glklmlllev isgerlpkpe rgkmrvhkin nvnkaldfia |
| 121 | skgvklvsig aeeivdgnak mtlgmiwtii lrfaiqdisv eetsakegll lwcqrktapy |
| 181 | knvnvqnfhi swkdglafna lihrhrpeli eydklrkddp vtnlnnafev aekyldipkm |
| 241 | ldaedivnta rpdekaimty vssfyhafsg aqkaetaanr ickvlavnqe nehlmedyek |
| 301 | lasdllewir rtipwledrv pqktiqemqq kledfrdyrr vhkppkvqek cqleinfntl |
| 361 | qtklrlsnrp afmpsegkmv sdinngwqhl eqaekgyeew llneirrler ldhlaekfrq |
| 421 | kasiheawtd gkeamlkhrd yetatlsdik alirkheafe sdlaahqdrv eqiaaiaqel |
| 481 | neldyydshn vntrcqkicd qwdalgslth srrealekte kqleaidqlh leyakraapf |
| 541 | nnwmesamed lqdmfivhti eeieglisah dqfkstlpda drereailai hkeaqriaes |
| 601 | nhiklsgsnp yttvtpqiin skwekvqqlv pkrdhallee qskqqsnehl rrqfasqanv |
| 661 | vgpwiqtkme eigrisiemn gtledqlshl kqyersivdy kpnldlleqq hqliqealif |
| 721 | dnkhtnytme hirvgweqll ttiartinev enqiltrdak gisqeqmqef rasfnhfdkd |
| 781 | hggalgpeef kaclislgyd vendrqgeae fnrimslvdp nhsglvtfqa fidfmsrett |
| 841 | dtdtadqvia sfkvlagdkn fitaeelrre lppdqaeyci armapyqgpd avpgaldyks |
| 901 | fstalygesd l |

Actinin alpha 4, isoform 2, NP_001308962.1 (SEQ ID NO: 32)

| | |
|---|---|
| 1 | mvdyhaanqs yqygpssagn gaggggsmgd ymaqeddwdr dllldpawek qqrktftawc |
| 61 | nshlrkagtq ienidedfrd glklmlllev isgerlpkpe rgkmrvhkin nvnkaldfia |
| 121 | skgvklvsig aeeivdgnak mtlgmiwtii lrfaiqdisv eetsakegll lwcqrktapy |
| 181 | knvnvqnfhi swkdglafna lihrhrpeli eydklrkddp vtnlnnafev aekyldipkm |
| 241 | ldaedivgtl rpdekaimty vscfyhafsg aqkaetaanr ickvlavnqe nehlmedyek |
| 301 | lasdllewir rtipwledrv pqktiqemqq kledfrdyrr vhkppkvqek cqleinfntl |
| 361 | qtklrlsnrp afmpsegkmv sdinngwqhl eqaekgyeew llneirrler ldhlaekfrq |
| 421 | kasiheawtd gkeamlkhrd yetatlsdik alirkheafe sdlaahqdrv eqiaaiaqel |
| 481 | neldyydshn vntrcqkicd qwdalgslth srrealekte kqleaidqlh leyakraapf |
| 541 | nnwmesamed lqdmfivhti eeieglisah dqfkstlpda drereailai hkeaqriaes |
| 601 | nhiklsgsnp yttvtpqiin skwekvqqlv pkrdhallee qskqqsnehl rrqfasqanv |
| 661 | vgpwiqtkme eigrisiemn gtledqlshl kqyersivdy kpnldlleqq hgliqealif |
| 721 | dnkhtnytme hirvgweqll ttiartinev enqiltrdak gisqeqmqef rasfnhfdkk |
| 781 | qtgsmdsddf rallistgys lgeaefnrim slvdpnhsgl vtfqafidfm srettdtdta |
| 841 | dqviasfkvl agdknfitae elrrelppdq aeyciarmap yqgpdavpga ldyksfstal |
| 901 | ygesdl |

Activin A receptor type 1, NP_001096.1, NP_001104537.1, NP_001334592.1,
NP_001334593.1, NP_001334594.1, NP_001334595.1, NP_001334596.1 (SEQ ID NO:
33)

| | |
|---|---|
| 1 | mvdgvmilpv limialpsps medekpkvnp klymcvcegl scgnedhceg qqcfsslsin |
| 61 | dgfhvyqkgc fqvyeqgkmt cktppspgqa veccqgdwcn rnitaqlptk gksfpgtqnf |
| 121 | hlevgliils vvfavcllac llgvalrkfk rrnqerlnpr dveygtiegl ittnvgdstl |
| 181 | adlldhscts gsgsglpflv qrtvarqitl lecvgkgryg evwrgswqge nvavkifssr |
| 241 | dekswfrete lyntvmlrhe nilgfiasdm tsrhsstqlw lithyhemgs lydylqlttl |
| 301 | dtvsclrivl siasglahlh ieifgtqgkp aiahrdlksk nilvkkngqc ciadlglavm |
| 361 | hsqstnqldv gnnprvgtkr ymapevldet iqvdcfdsyk rvdiwafglv lwevarrmvs |
| 421 | ngivedykpp fydvvpndps fedmrkvvcv dqqrpnipnr wfsdptltsl aklmkecwyq |
| 481 | npsarltalr ikktltkidn sldklktdc |

Alcohol dehydrogenase 1C (class I), gamma polypeptide, NP_000660.1 (SEQ ID
NO: 34)

| | |
|---|---|
| 1 | mstagkvikc kaavlwelkk pfsieeveva ppkahevrik mvaagicrsd ehvvsgnlvt |
| 61 | plpvilghea agivesvgeg vttvkpgdkv iplftpqcgk cricknpesn yclkndlgnp |
| 121 | rgtlqdgtrr ftcsgkpihh fvgvstfsqy tvvdenavak idaasplekv cligcgfstg |
| 181 | ygsavkvakv tpgstcavfg lggvglsvvm gckaagaari iavdinkdkf akakelgate |
| 241 | cinpqdykkp iqevlkemtd ggvdfsfevi grldtmmasl lccheacgts vivgvppdsq |
| 301 | nlsinpmlll tgrtwkgaif ggfkskesvp klvadfmakk fsldalitni lpfekinegf |
| 361 | dllrsgksir tvltf |

Adenosine A2a receptor, NP_000666.2, NP_001265426.1, NP_001265427.1,
NP_001265428.1, NP_ 001265429.1 (SEQ ID NO: 35)

| | |
|---|---|
| 1 | mpimgssvyi tvelaiavla ilgnvlvowa vwlnsnlqnv tnyfvvslaa adiavgvlai |
| 61 | pfaitistgf caachgclfi acfvlvltqs sifsllaiai dryiairipl rynglvtgtr |
| 121 | akgiiaicwv lsfaigltpm lgwnncgqpk egknhsqgcg egqvaclfed vvpmnymvyf |
| 181 | nffacvlvpl llmlgvylri flaarrqlkg mesqplpger arstlqkevh aakslaiivg |
| 241 | lfalcwlplh iincftffcp dcshaplwlm ylaivlshtn svvnpfiyay rirefrqtfr |
| 301 | kiirshvlrq qepfkaagts arvlaahgsd geqvslrlng hppgvwangs aphperrpng |
| 361 | yalglvsggs aqesqgntgl pdvellshel kgvcpeppgl ddplaqdgag vs |

Rho guanine nucleotide exchange factor 16, NP_055263.2 (SEQ ID NO: 36)

| | |
|---|---|
| 1 | maqrhsdssl eekllghrfh selrldaggn pasglpmvrg sprvrddaaf qpqvpappqp |
| 61 | rppgheepwp ivlstespaa lklgtqqlip kslavaskak tparhqsfga avlsreaarr |
| 121 | dpkllpapsf slddmdvdkd pggmlrrnlr nqsyraamkg lgkpggqgda iqlspklqal |
| 181 | aeepsqphtr spaknkktlg rkrghkgsfk ddpqlyqeiq erglntsqes dddildesss |
| 241 | pegtqkvdat ivvksyrpaq vtwsqlpevv elgildqlst eerkrqeamf eiltsefsyq |
| 301 | hslsilveef lqskelratv tqmehhhlfs nildvlgasq rffedleqrh kaqvlvedis |
| 361 | dileehaekh fhpyiaycsn evyqqrtlqk lissnaafre alreierrpa cgglpmlsfl |
| 421 | ilpmqrvtrl pllmdtlclk tqghseryka asralkaisk lvrqcnegah rmermeqmyt |
| 481 | lhtqldfskv kslplisasr wllkrgelfl veetglfrki asrptcylfl fndvlvvtkk |
| 541 | kseesymvqd yaqmnhiqve kiepselplp gggnrsssvp hpfqvtllrn segrqeqlll |
| 601 | ssdsasdrar wivalthser qwqglsskgd lpqveitkaf fakqadevtl qqadvvlvlq |
| 661 | qedgwlyger lrdgetgwfp edfarfitsr vavegnvrrm erlrvetdv |

B-cell linker, isoform 1, NP_037446.1 (SEQ ID NO: 37)

| | |
|---|---|
| 1 | mdklnkitvp asqklrqlqk mvhdiknneg gimnkikklk vkappsvprr dyasespade |
| 61 | eeqwsddfds dyenpdehsd semyvmpaee naddsyeppp veqetrpvhp alpfargeyi |
| 121 | dnrssqrhsp pfsktlpskp swpsekarlt stlpaltalq kpqvppkpkg lledeadyvv |
| 181 | pvedndenyi hptesssppp ekapmvnrst kpnsstpasp pgtasgrnsg awetkspppa |
| 241 | apsplpragk kpttplkttp vasqqnassv ceekpipaer hrgsshrqea vqspvfppaq |
| 301 | kqihqkpipl prfteggnpt vdgplpsfss nstiseqeag vlckpwyaga cdrksaeeal |
| 361 | hrsnkdgsfl irkssghdsk qpytlvvffn krvynipvrf ieatkqyalg rkkngeeyfg |
| 421 | svaeiirnhq hsplvlidsq nntkdstrlk yavkvs |

B-cell linker, isoform 2, NP_001107566.1 (SEQ ID NO: 38)

| | |
|---|---|
| 1 | mdklnkitvp asqklrqlqk mvhdiknneg gimnkikklk vkappsvprr dyasespade |
| 61 | eeqwsddfds dyenpdehsd semyvmpaee naddsyeppp veqetrpvhp alpfargeyi |
| 121 | dnrssqrhsp pfsktlpskp swpsekarlt stlpaltalq kpqvppkpkg lledeadyvv |
| 181 | pvedndenyi hptesssppp ekgrnsgawe tkspppaaps plpragkkpt tplkttpvas |
| 241 | qqnassvcee kpipaerhrg sshrqeavqs pvfppaqkqi hqkpiplprf teggnptvdg |
| 301 | plpsfssnst iseqeagvlc kpwyagacdr ksaeealhrs nkdgsflirk ssghdskqpy |
| 361 | tlvvffnkrv ynipvrfiea tkqyalgrkk ngeeyfgsva eiirnhqhsp lvlidsqnnt |
| 421 | kdstrlkyav kvs |

B-cell linker, isoform 3, NP_001245369.1 (SEQ ID NO: 39)

| | |
|---|---|
| 1 | mdklnkitvp asqklrqlqk mvhdiknneg gimnkikklk vkappsvprr dyasespade |
| 61 | eeqwsddfds dyenpdehsd semyvmpaee naddsyeppp veqetrpvhp alpfargeyi |
| 121 | dnrssqrhsp pfsktlpskp swpsekarlt stlpaltalq kpqvppkpkg lledeadyvv |
| 181 | pvedndenyi hptesssppp ekapmvnrst kpnsstpasp pgtasgrnsg awetkspppa |
| 241 | apsplpragk kpttplkttp vasqqnassv ceekpipaer hrgsshrqea vqspvfppaq |
| 301 | kqihqkpipl prfteggnpt vdgplpsfss nstiseqeag vlckpwyaga cdrksaeeal |
| 361 | hrsnkyfgsv aeiirnhqhs plvlidsqnn tkdstrlkya vkvs |

B-cell linker, isoform 4, NP_001245370.1 (SEQ ID NO: 40)

| | |
|---|---|
| 1 | mdklnkitvp asqklrqlqk mvhdiknneg gimnkikklk vkappsvprr dyasespade |
| 61 | eeqwsddfds dyenpdehsd semyvmpaee naddsyeppp veqetrpvhp alpfargeyi |
| 121 | dnrssqrhsp pfsktlpskp swpsekarlt stlpaltalq kpqvppkpkg lledeadyvv |
| 181 | pvedndenyi hptesssppp ekgrnsgawe tkspppaaps plpragkkpt tplkttpvas |
| 241 | qqnassvcee kpipaerhrg sshrqeavqs pvfppaqkqi hqkpiplprf teggnptvdg |
| 301 | plpsfssnst iseqeagvlc kpwyagacdr ksaeealhrs nkyfgsvaei irnhqhsplv |
| 361 | lidsqnntkd strlkyavkv s |

B-cell linker, isoform 5, NP_001245371.1 (SEQ ID NO: 41)

| | |
|---|---|
| 1 | mdklnkitvp asqklrqlqk mvhdiknneg gimnkikklk vkappsvprr dyasespade |
| 61 | eeqwsddfds dyenpdehsd semyvmpaee naddsyeppp veqetrpvhp alpfargtas |
| 121 | grnsgawetk spppaapspl pragkkpttp lkttpvasqq nassvceekp ipaerhrgss |
| 181 | hrqeavqspv fppaqkqihq kpiplprfte ggnptvdgpl psfssnstis eqeagvlckp |
| 241 | wyagacdrks aeealhrsnk yfgsvaeiir nhqhsplvli dsqnntkdst rlkyavkvs |

Basonuclin 1, isoform a, NP_001708.3 (SEQ ID NO: 42)

| | |
|---|---|
| 1 | mrrrppsrgg rgaararetr rqprhrsgrr maeaisctln cscqsfkpgk inhrqcdqck |
| 61 | hgwvahalsk lrippmypts qveivqsnvv fdisslmlyg tqaipvrlki lldrlfsvlk |
| 121 | qdevlqilha ldwtlqdyir gyvlqdasgk vldhwsimts eeevatlqqf lrfgetksiv |
| 181 | elmaiqekee qsiiippsta nvdirafies cshrssslpt pvdkgnpssi hpfenlisnm |
| 241 | tfmlpfqffn plppaligsl peqymleqgh dqsqdpkqev hgpfpdssfl tssstpfqve |
| 301 | kdqclncpda itkkedsthl sdsssynivt kfertqlspe akvkpernsl gtkkgrvfct |
| 361 | acektfydkg tlkihynavh lkikhkctie gcnmvfsslr srnrhsanpn prlhmpmnrn |
| 421 | nrdkdlrnsl nlassenykc pgftvtspdc rpppsypgsg edskgqpafp nigqngvlfp |
| 481 | nlktvqpvlp fyrspatpae vantpgilps lpllsssipe qlisnempfd alpkkksrks |
| 541 | smpikiekea veianekrhn lssdedmplq vvsedeqeac spqshrvsee qhvqsgglgk |
| 601 | pfpegerpch resviessga isqtpeqath nsereteqtp alimvpreve dgghehyftp |
| 661 | gmepqvpfsd ymelqqrlla gglfsalsnr gmafpcleds kelehvgqha larqieenrf |
| 721 | qcdickktfk nacsvkihhk nmhvkemhtc tvegcnatfp srrsrdrhss nlnlhqkals |
| 781 | qealessedh fraayllkdv akeayqdvaf tqqasqtsvi fkgtsrmgsl vypitqvhsa |
| 841 | slesynsgpl segtildlst tssmksesss hsswdsdgvs eegtvlmeds dgncegsslv |
| 901 | pgedeypicv lmekadqsla slpsglpitc hlcqktysnk gtfrahyktv hlrqlhkckv |
| 961 | pgcntmfssv rsrnrhsqnp nlhkslassp shlq |

Basonuclin 1, isoform b, NP_001288135.1 (SEQ ID NO: 43)

| | |
|---|---|
| 1 | mrcrnmffsf kaslcgcgaa tapsltaisc tlncscqsfk pgkinhrqcd qckhgwvaha |
| q | lsklrippmy ptsqveivqs nvvfdisslm lygtqaipvr lkilldrlfs vlkqdevlqi |
| 121 | lhaldwtlqd yirgyvlqda sgkvldhwsi mtseeevatl qqflrfgetk sivelmaiqe |
| 181 | keeqsiiipp stanvdiraf iescshrsss lptpvdkgnp ssihpfenli snmtfmlpfq |
| 241 | ffnplppali gslpeqymle qghdqsqdpk qevhgpfpds sfltssstpf qvekdqclnc |
| 301 | pdaitkkeds thlsdsssyn ivtkfertql speakvkper nslgtkkgrv fctacektfy |
| 361 | dkgtlkihyn avhlkikhkc tiegcnmvfs slrsrnrhsa npnprlhmpm nrnnrdkdlr |
| 421 | nslnlassen ykcpgftvts pdcrpppsyp gsgedskgqp afpnigqngv lfpnlktvqp |
| 481 | vlpfyrspat paevantpgi lpslpllsss ipeqlisnem pfdalpkkks rkssmpikie |
| 541 | keaveianek rhnlssdedm plqvvsedeq eacspqshrv seeqhvgsgg lgkpfpeger |
| 601 | pchresvies sgaisqtpeq athnserete qtpalimvpr evedgghehy ftpgmepqvp |
| 661 | fsdymelqqr llagglfsal snrgmafpcl edskelehvg qhalarqiee nrfqcdickk |
| 721 | tfknacsvki hhknmhvkem htctvegcna tfpsrrsrdr hssnlnlhqk alsqealess |
| 781 | edhfraayll kdvakeayqd vaftqqasqt svifkgtsrm gslvypitqv hsaslesyns |
| 841 | gplsegtild lsttssmkse ssshsswdsd gvseegtvlm edsdgncegs slvpgedeyp |
| 901 | icvlmekadq slaslpsglp itchlcqkty snkgtfrahy ktvhlrqlhk ckvpgcntmf |
| 961 | ssvrsrnrhs qnpnlhksla sspshlq |

BPI fold containing family A member 1, precursor, NP_001230122.1,
NP_057667.1, NP_570913.1 (SEQ ID NO: 44)

| | |
|---|---|
| 1 | mfqtgglivf ygllaqtmaq fgglpvpldq tlplnvnpal plsptglags ltnalsngll |
| 61 | sggllgilen lplldilkpg ggtsggllgg llgkvtsvip glnniidikv tdpqllelgl |
| 121 | vqspdghrly vtiplgiklq vntplvgasl lrlavkldit aeilavrdkq erihlvlgdc |
| 181 | thspgslqis lldglgplpi qglldsltgi lnkvlpelvq gnvcplvnev lrglditlvh |
| 241 | divnmlihgl qfvikv |

Calcium voltage-gated channel auxiliary subunit beta 3, isoform 1,
NP_000716.2 (SEQ ID NO: 45)

| | |
|---|---|
| 1 | myddsyvpgf edseagsads ytsrpsldsd vsleedresa rrevesqaqq qlerakhkpv |
| 61 | afavrtnvsy cgvldeecpv qgsgvnfeak dflhikekys ndwwigrlvk eggdiafips |
| 121 | pqrlesirlk qeqkarrsgn psslsdignr rspppslakq kqkqaehvpp ydvvpsmrpv |
| 181 | vlvgpslkgy evtdmmqkal fdflkhrfdg risitrvtad lslakrsvin npgkrtiier |
| 241 | ssarssiaev qseierifel akslqlvvld adtinhpaql aktslapiiv fvkvsspkvl |
| 301 | qrlirsrgks qmkhltvqmm aydklvqcpp esfdvilden qledacehla eylevywrat |
| 361 | hhpapgpgll gppsaipglq nqqllgerge ehsplerdsl mpsdeasess rqawtgssqr |
| 421 | ssrhleedya dayqdlyqph rqhtsglpsa nghdpqdrll aqdsehnhsd rnwqrnrpwp |
| 481 | kdsy |

Calcium voltage-gated channel auxiliary subunit beta 3, isoform 2,
NP_001193844.1 (SEQ ID NO: 46)

| | |
|---|---|
| 1 | myddsyvpgf edseagsads ytsrpsldsd vsleedresa rrevesqaqq qlerakkysn |
| 61 | dwwigrlvke ggdiafipsp qrlesirlkq eqkarrsgnp sslsdignrr spppslakqk |
| 121 | qkqaehvppy dvvpsmrpvv lvgpslkgye vtdmmqkalf dflkhrfdgr isitrvtadl |
| 181 | slakrsvlnn pgkrtiiers sarssiaevq seierifela kslqlvvlda dtinhpaqla |
| 241 | ktslapiivf vkvsspkvlq rlirsrgksq mkhltvqmma ydklvqcppe sfdvildenq |
| 301 | ledacehlae ylevywrath hpapgpgllg ppsaipglqn qqllgergee hsplerdslm |
| 361 | psdeasessr qawtgssqrs srhleedyad ayqdlyqphr qhtsglpsan ghdpqdrlla |
| 421 | qdsehnhsdr nwqrnrpwpk dsy |

Calcium voltage-gated channel auxiliary subunit beta 3, isoform 3,
NP_001193845.1 (SEQ ID NO: 47)

| | |
|---|---|
| 1 | msfsdssatf llnegsadsy tsrpsldsdv sleedresar revesqaqqq lerakhkpva |
| 61 | favrtnvsyc gvldeecpvq gsgvnfeakd flhikekysn dwwigrlvke ggdiafipsp |
| 121 | qrlesirlkq eqkarrsgnp sslsdignrr spppslakqk qkqaehvppy dvvpsmrpvv |
| 181 | lvgpslkgye vtdmmqkalf dflkhrfdgr isitrvtadl slakrsvlnn pgkrtiiers |
| 241 | sarssiaevq seierifela kslqlvvlda dtinhpaqla ktslapiivf vkvsspkvlq |
| 301 | rlirsrgksq mkhltvqmma ydklvqcppe sfdvildenq ledacehlae ylevywrath |
| 361 | hpapgpgllg ppsaipglqn qqllgergee hsplerdslm psdeasessr qawtgssqrs |
| 421 | srhleedyad ayqdlyqphr qhtsglpsan ghdpqdrlla qdsehnhsdr nwqrnrpwpk |
| 481 | dsy |

Calcium voltage-gated channel auxiliary subunit beta 3, isoform 4,
NP_001193846.1 (SEQ ID NO: 48)

| | |
|---|---|
| 1 | megsadsyts rpsldsdvsl eedresarre vesqaqqqle rakhkpvafa vrtnvsycgv |
| 61 | ldeecpvggs gvnfeakdfl hikekysndw wigrlvkegg diafipspqr lesirlkqeq |
| 121 | karrsgnpss lsdignrrsp ppslakqkqk qaehvppydv vpsmrpvvlv gpslkgyevt |
| 181 | dmmqkalfdf lkhrfdgris itrvtadlsl akrsvlnnpg krtiierssa rssiaevqse |
| 241 | ierifelaks lqlvvldadt inhpaqlakt slapiivfvk vsspkvlqrl irsrgksqmk |
| 301 | hltvqmmayd klvqcppesf dvildenqle dacehlaeyl evywrathhp apgpgllgpp |
| 361 | saipglqnqq llgergeehs plerdslmps deasessrqa wtgssqrssr hleedyaday |
| 421 | qdlyqphrqh tsglpsangh dpqdrllaqd sehnhsdrnw qrnrpwpkds y |

Caspase 3, preproprotein, NP_ 001341706.1, NP_001341707.1, NP_004346.3,
NP_116786.1 (SEQ ID NO: 49)

| | |
|---|---|
| 1 | mentensvds ksiknlepki ihgsesmdsg isldnsykmd ypemglciii nnknfhkstg |
| 61 | mtsrsgtdvd aanlretfrn lkyevrnknd ltreeivelm rdvskedhsk rssfvcvlls |
| 121 | hgeegiifgt ngpvdlkkit nffrgdrcrs ltgkpklfii qacrgteldc gietdsgvdd |
| 181 | dmachkipve adflyaysta pgyyswrnsk dgswfiqslc amlkqyadkl efmhiltrvn |
| 241 | rkvatefesf sfdatfhakk qipcivsmlt kelyfyh |

Caspase 3, isoform b, NP_001341708.1, NP001341709.1 (SEQ ID NO: 50)

| | |
|---|---|
| 1 | mdsgisldns ykmdypemgl ciiinnknfh kstgmtsrsg tdvdaanlre tfrnlkyevr |
| 61 | nkndltreei velmrdvske dhskrssfvc vllshgeegi ifgtngpvdl kkitnffrgd |
| 121 | rcrsltgkpk lfiiqacrgt eldcgietds gvdddmachk ipveadflya ystapgyysw |
| 181 | rnskdgswfi qslcamlkqy adklefmhil trvnrkvate fesfsfdatf hakkqipciv |
| 241 | smltkelyfy h |

Caspase 3, isoform c, NP_001341710.1, NP001341711.1 (SEQ ID NO: 51)

| | |
|---|---|
| 1 | mentensvds ksiknlepki ihgsesmdsg isldnsykmd ypemglciii nnknfhkstg |
| 61 | mtsrsgtdvd aanlretfrn lkyevrnknd ltreeivelm rdvskedhsk rssfvcvlls |
| 121 | hgeegiifgt ngpvdlkkit nffrgdrcrs ltgkpklfii qviilgeiqr mapgsssrfv |
| 181 | pc |

Caspase 3, isoform d, NP_001341712.1 (SEQ ID NO: 52)

| | |
|---|---|
| 1 | msdalikvsm entensvdsk siknlepkii hgsesmdsgi sldnsykmdy pemglciiin |
| 61 | nknfhkstgm tsrsgtdvda anlretfrnl kyevrnkndl treeivelmr dvskedhskr |
| 121 | ssfvcvllsh geegiifgtn gpvdlkkitn ffrgdrcrsl tgkpklfiiq viilgeiqrm |
| 181 | apgsssrfvp c |

Caspase 3, isoform e, NP_001341713.1 (SEQ ID NO: 53)

| | |
|---|---|
| 1 | mdsgisldns ykmdypemgl ciiinnknfh kstgmtsrsg tdvdaanlre tfrnlkyevr |
| 61 | nkndltreei velmrdvske dhskrssfvc vllshgeegi ifgtngpvdl kkitnffrgd |
| 121 | rcrsltgkpk lfiiqviilg eiqrmapgss srfvpc |

Caveolin 1, isoform alpha, NP_001744.2 (SEQ ID NO: 54)

| | |
|---|---|
| 1 | msggkyvdse ghlytvpire qgniykpnnk amadelsekq vydahtkeid lvnrdpkhln |
| 61 | ddvvkidfed viaepegths fdgiwkasft tftvtkywfy rllsalfgip maliwgiyfa |
| 121 | ilsflhiwav vpciksflie iqcisrvysi yvhtvcdplf eavgkifsnv rinlqkei |

Caveolin 1, isoform beta, NP_001166366.1, NP_001166367.1, NP_001166368.1 (SEQ
ID NO: 55)

| | |
|---|---|
| 1 | madelsekqv ydahtkeidl vnrdpkhlnd dvvkidfedv iaepegthsf dgiwkasftt |
| 61 | ftvtkywfyr llsalfgipm aliwgiyfai lsflhiwavv pciksfliei qcisrvysiy |
| 121 | vhtvcdplfe avgkifsnvr inlqkei |

Cadherin 1, isoform 1 preproprotein, NP_004351.1 (SEQ ID NO: 56)

| | |
|---|---|
| 1 | mgpwsrslsa lllllqvssw lcqepepchp gfdaesytft vprrhlergr vlgrvnfedc |
| 61 | tgrqrtayfs ldtrfkvgtd gvitvkrplr fhnpqihflv yawdstyrkf stkvtlntvg |
| 121 | hhhrppphqa sysgiqaell tfpnsspglr rqkrdwvipp iscpenekgp fpknlvqiks |
| 181 | nkdkegkvfy sitgqgadtp pvgvfiiere tgwlkvtepl dreriatytl fshayssngn |
| 241 | avedpmeili tvtdqndnkp eftqevfkgs vmegalpgts vmevtatdad ddvntynaai |
| 301 | aytilsqdpe lpdknmftin rntgvisvvt tgldresfpt ytlvvqaadl qgeglsttat |
| 361 | avitvtdtnd nppifnptty kgqvpenean vvittlkvtd adapntpawe avytilnddg |
| 421 | gqfvvttnpv nndgilktak gldfeakqqy ilhvavtnvv pfevslttst atvtvdvldv |
| 481 | neapifvppe krvevsedfg vgqeitsyta qepdtfmeqk ityriwrdta nwleinpdtg |
| 541 | aistraeldr edfehvknst ytaliiatdn gspvatgtgt lllilsdvnd napipeprti |
| 601 | ffcernpkpq viniidadlp pntspftael thgasanwti qyndptqesi ilkpkmalev |
| 661 | gdykinlklm dnqnkdqvtt levsvcdceg aagvcrkaqp veaglqipai lgilggilal |
| 721 | lililllllf lrrravvkep llppeddtrd nvyyydeegg geedqdfdls qlhrgldarp |
| 781 | evtrndvapt lmsvprylpr panpdeignf idenlkaadt dptappydsl lvfdyegsgs |
| 841 | eaaslsslns sesdkdqdyd ylnewgnrfk kladmyggge dd |

Cadherin 1, isoform 2 precursor, NP_001304113.1 (SEQ ID NO: 57)

| | |
|---|---|
| 1 | mgpwsrslsa lllllqvssw lcqepepchp gfdaesytft vprrhlergr vlgrvnfedc |
| 61 | tgrqrtayfs ldtrfkvgtd gvitvkrplr fhnpqihflv yawdstyrkf stkvtlntvg |
| 121 | hhhrppphqa sysgiqaell tfpnsspglr rqkrdwvipp iscpenekgp fpknlvqiks |
| 181 | nkdkegkvfy sitgqgadtp pvgvfiiere tgwlkvtepl dreriatytl fshavssngn |
| 241 | avedpmeili tvtdqndnkp eftqevfkgs vmegalpgts vmevtatdad ddvntynaai |
| 301 | aytilsqdpe lpdknmftin rntgvisvvt tgldresfpt ytlvvqaadl qgeglsttat |
| 361 | avitvtdtnd nppifnpttg ldfeakqqyi lhvavtnvvp fevslttsta tvtvdvldvn |
| 421 | eapifvppek rvevsedfgv gqeitsytaq epdtfmeqki tyriwrdtan wleinpdtga |
| 481 | istraeldre dfehvknsty taliiatdng spvatgtgtl llilsdvndn apipeprtif |
| 541 | fcernpkpqv iniidadlpp ntspftaelt hgasanwtiq yndptqesii lkpkmalevg |
| 601 | dykinlklmd nqnkdqvttl evsvcdcega agvcrkaqpv eaglqipail gilggilall |
| 661 | ililllllf1 rrravvkepl lppeddtrdn vyyydeeggg eedqdfdlsq lhrgldarpe |
| 721 | vtrndvaptl msvprylprp anpdeignfi denlkaadtd ptappydsll vfdyegsgse |
| 781 | aaslsslnss esdkdqdydy lnewgnrfkk ladmyggged d |

Cadherin 1, isoform 3, NP_001304114.1 (SEQ ID NO: 58)

| | |
|---|---|
| 1 | meqkityriw rdtanwlein pdtgaistra eldredfehv knstytalii atdngspvat |
| 61 | gtgtlllils dvndnapipe prtiffcern pkpqviniid adlppntspf taelthgasa |
| 121 | nwtiqyndpt qesiilkpkm alevgdykin lklmdnqnkd qvttlevsvc dcegaagvcr |
| 181 | kaqpveaglq ipailgilgg ilallilill lllflrrrav vkepllpped dtrdnvyyyd |
| 241 | eegggeedqd fdlsqlhrgl darpevtrnd vaptlmsvpr ylprpanpde ignfidenlk |
| 301 | aadtdptapp ydsllvfdye gsgseaasls slnssesdkd qdydylnewg nrfkkladmy |
| 361 | gggedd |

Cadherin 1, isoform 4, NP_001304115.1 (SEQ ID NO: 59)

| | |
|---|---|
| 1 | malevgdyki nlklmdnqnk dqvttlevsv cdcegaagvc rkaqpveagl qipailgilg |
| 61 | gilallilil llllflrrra vvkepllppe ddtrdnvyyy deegggeedq dfdlsqlhrg |
| 121 | ldarpevtrn dvaptlmsvp rylprpanpd eignfidenl kaadtdptap pydsllvfdy |
| 181 | egsgseaasl sslnssesdk dqdydylnew gnrfkkladm ygggedd |

Cytochrome c oxidase subunit 8C, NP_892016.1 (SEQ ID NO: 60)

| | |
|---|---|
| 1 | mpllrgrcpa rrhyrrlall glqpaprfah sgpprqrpls aaemavglvv ffttfltpaa |
| 61 | yvlgnlkqfr rn |

Carnitine palmitoyltransferase 1A, isoform 1, NP_001867.2 (SEQ ID NO: 61)

| | |
|---|---|
| 1 | maeahqavaf qftvtpdgid lrlshealrq iylsglhswk kkfirfkngi itgvypasps |
| 61 | swlivvvgvm ttmyakidps lgiiakinrt letancmssq tknvvsgvlf gtglwvaliv |
| 121 | tmryslkvll syhgwmfteh gkmsratkiw mgmvkifsgr kpmlysfqts lprlpvpavk |
| 181 | dtvnrylqsv rplmkeedfk rmtalaqdfa vglgprlqwy lklkswwatn yvsdwweeyi |
| 241 | ylrgrgplmv nsnyyamdll yilpthiqaa ragnaihail lyrrkldree ikpirllgst |
| 301 | iplcsaqwer mfntsripge etdtiqhmrd skhivvyhrg ryfkvwlyhd grllkpreme |
| 361 | qqmqrildnt sepqpgearl aaltagdrvp warcrqayfg rgknkqslda vekaaffvtl |
| 421 | deteegyrse dpdtsmdsya ksllhgrcyd rwfdksftfv vfkngkmgln aehswadapi |
| 481 | vahlweyvms idslqlgyae dghckgdinp nipyptrlqw dipgecqevi etslntanll |
| 541 | andvdfhsfp fvafgkgiik kcrtspdafv qlalqlahyk dmgkfcltye asmtrlfreg |
| 601 | rtetvrsctt escdfvramv dpaqtveqrl klfklasekh qhmyrlamtg sgidrhlfcl |
| 661 | yvvskylave spflkevlse pwrlstsqtp qqqvelfdle nnpeyvssgg gfgpvaddgy |
| 721 | gvsyilvgen linfhisskf scpetdshrf grhlkeamtd iitlfglssn skk |

Carnitine palmitoyltransferase 1A, isoform 2, NP_001027017.1 (SEQ ID NO: 62)

| | |
|---|---|
| 1 | maeahqavaf qftvtpdgid lrlshealrq iylsglhswk kkfirfkngi itgvypasps |
| 61 | swlivvvgvm ttmyakidps lgiiakinrt letancmssq tknvvsgvlf gtglwvaliv |
| 121 | tmryslkvll syhgwmfteh gkmsratkiw mgmvkifsgr kpmlysfqts lprlpvpavk |
| 181 | dtvnrylqsv rplmkeedfk rmtalaqdfa vglgprlqwy lklkswwatn yvsdwweeyi |
| 241 | ylrgrgplmv nsnyyamdll yilpthiqaa ragnaihail lyrrkldree ikpirllgst |
| 301 | iplcsaqwer mfntsripge etdtiqhmrd skhivvyhrg ryfkvwlyhd grllkpreme |
| 361 | qqmqrildnt sepqpgearl aaltagdrvp warcrqayfg rgknkqslda vekaaffvtl |
| 421 | deteegyrse dpdtsmdsya ksllhgrcyd rwfdksftfv vfkngkmgln aehswadapi |
| 481 | vahlweyvms idslqlgyae dghckgdinp nipyptrlqw dipgecqevi etslntanll |
| 541 | andvdfhsfp fvafgkgiik kcrtspdafv qlalqlahyk dmgkfcltye asmtrlfreg |
| 601 | rtetvrsctt escdfvramv dpaqtveqrl klfklasekh qhmyrlamtg sgidrhlfcl |
| 661 | yvvskylave spflkevlse pwrlstsqtp qqqvelfdle nnpeyvssgg gfgpvaddgy |
| 721 | gvsyilvgen linfhisskf scpetgiisq gpssdt |

Cancer/testis antigen 1A, NP_640343.1 (SEQ ID NO: 63)

| | |
|---|---|
| 1 | mqaegrgtgg stgdadgpgg pgipdgpggn aggpgeagat ggrgprgaga arasgpggga |
| 61 | prgphggaas glngccrcga rgpesrllef ylampfatpm eaelarrsla qdapplpvpg |
| 121 | vllkeftvsg niltirltaa dhrqlqlsis sclqqlsllm witqcflpvf laqppsgqrr |

C-X-C motif chemokine ligand 13, NP_006410.1 (SEQ ID NO: 64)

| | |
|---|---|
| 1 | mkfistslll mllvsslspv qgvlevyyts lrcrcvqess vfiprrfidr iqilprgngc |
| 61 | prkeiivwkk nksivcvdpq aewiqrmmev lrkrssstlp vpvfkrkip |

Diacylglycerol kinase eta, isoform 1, NP_001191433.1, NP_690874.2 (SEQ ID NO:
65)

| | |
|---|---|
| 1 | magaggqhhp pgaaggaaag agaavtsaaa sagpgedssd seaeqegpqk lirkvstsgq |
| 61 | irtktsikeg qllkqtssfq rwkkryfklr grtlyyakds kslifdevdl sdasvaeast |
| 121 | knannsftii tpfrrlmlca enrkemedwi sslksvqtre pyevaqfnve hfsgmhnwya |
| 181 | csharptfcn vcreslsgvt shglscevck fkahkrcavr atnnckwttl asigkdiied |
| 241 | edgvamphqw legnlpvsak cavcdktcgs vlrlqdwkcl wcktmvhtac kdlyhpicp1 |
| 301 | gqckvsiipp ialnstdsdg fcratfsfcv spllvfvnsk sgdnqgvkfl rrfkqllnpa |
| 361 | qvfdlmnggp hlglrlfqkf dnfrilvcgg dgsvgwvlse idklnlnkqc qlgvlplgtg |
| 421 | ndlarvlgwg gsydddtqlp qilekleras tkmldrwsim tyelklppka sllpgppeas |
| 481 | eefymtiyed svathltkil nsdehavvis saktlcetvk dfvakvekty dktlenavva |
| 541 | davaskcsvl nekleqllqa lhtdsqaapv lpglsplive edavesssee slgeskeqlg |
| 601 | ddvtkpssqk avkpreimlr anslkkavrq vieeagkvmd dptvhpcepa nqssdydste |
| 661 | tdeskeeakd dgakesitvk taprspdara syghsqtdsv pgpavaaske nlpvintrii |
| 721 | cpglraglaa siagssiink mllanidpfg atpfidpdld svdgysekcv mnnyfgigld |
| 781 | akislefnnk reehpekcrs rtknlmwygv lgtrellqrs yknleqrvql ecdgqyiplp |
| 841 | slqgiavini psyaggtnfw ggtkeddifa apsfddkile vvaifdsmqm aysrviklqh |
| 901 | hriaqcrtvk itifgdegvp vqvdgeawvq ppgiikivhk nraqmltrdr afestlkswe |
| 961 | dkqkcdsgkp vlrthlyihh aidlateevs qmqlcsqaae elitricdaa tihcllegel |
| 1021 | ahavnacsha lnkanprcpe sltrdtatei ainvkalyne tesllvgrvp lqlespheer |
| 1081 | vsnalhsvev elqklteipw lyyilhpned eeppmdctkr nnrstvfriv pkfkkekvqk |
| 1141 | qktssqpgsg dtesgscean spgn |

Diacylglycerol kinase eta, isoform 2, NP_821077.1 (SEQ ID NO: 66)

| | |
|---|---|
| 1 | magaggqhhp pgaaggaaag agaavtsaaa sagpgedssd seaeqegpqk lirkvstsgq |
| 61 | irtktsikeg qllkqtssfq rwkkryfklr grtlyyakds kslifdevdl sdasvaeast |
| 121 | knannsftii tpfrrlmlca enrkemedwi sslksvqtre pyevaqfnve hfsgmhnwya |
| 181 | csharptfcn vcreslsgvt shglscevck fkahkrcavr atnnckwttl asigkdiied |
| 241 | edgvamphqw legnlpvsak cavcdktcgs vlrlqdwkcl wcktmvhtac kdlyhpicp1 |
| 301 | gqckvsiipp ialnstdsdg fcratfsfcv spllvfvnsk sgdnqgvkfl rrfkqllnpa |
| 361 | qvfdlmnggp hlglrlfqkf dnfrilvcgg dgsvgwvlse idklnlnkqc qlgvlplgtg |
| 421 | ndlarvlgwg gsydddtqlp qilekleras tkmldrwsim tyelklppka sllpgppeas |
| 481 | eefymtiyed svathltkil nsdehavvis saktlcetvk dfvakvekty dktlenavva |
| 541 | davaskcsvl nekleqllqa lhtdsqaapv lpglsplive edavesssee slgeskeqlg |
| 601 | ddvtkpssqk avkpreimlr anslkkavrq vieeagkvmd dptvhpcepa nqssdydste |
| 661 | tdeskeeakd dgakesitvk taprspdara syghsqtdsv pgpavaaske nlpvintrii |
| 721 | cpglraglaa siagssiink mllanidpfg atpfidpdld svdgysekcv mnnyfgigld |
| 781 | akislefnnk reehpekcrs rtknlmwygv lgtrellqrs yknleqrvql ecdgqyiplp |
| 841 | slqgiavini psyaggtnfw ggtkeddifa apsfddkile vvaifdsmqm aysrviklqh |
| 901 | hriaqcrtvk itifgdegvp vqvdgeawvq ppgiikivhk nraqmltrdr afestlkswe |
| 961 | dkqkcdsgkp vlrthlyihh aidlateevs qmqlcsqaae elitricdaa tihclleqel |
| 1021 | ahavnacsha lnkanprcpe sltrdtatei ainvkalyne tesllvgrvp lqlespheer |
| 1081 | vsnalhsvev elqklteipw lyyilhpned eeppmdctkr nnrstvfriv pkfkkekvqk |
| 1141 | qktssqpvqk wgteevaawl dllnlgeykd ifirhdirga ellhlerrdl kdlgipkvgh |
| 1201 | vkrilqgike lgrstpqsev |

Diacylglycerol kinase eta, isoform 3, NP_001191434.1 (SEQ ID NO: 67)

| | |
|---|---|
| 1 | mlcaenrkem edwisslksv qtrepyevaq fnvehfsgmh nwyacsharp tfcnvcresl |
| 61 | sgvtshglsc evckfkahkr cavratnnck wttlasigkd iiededgvam phqwlegnlp |
| 121 | vsakcavcdk tcgsvlrlqd wkclwcktmv htackdlyhp icplgqckvs iippialnst |
| 181 | dsdgfcratf sfcvspllvf vnsksgdnqg vkflrrfkql lnpaqvfdlm nggphlglrl |
| 241 | fqkfdnfril vcggdgsvgw vlseidklnl nkqcqlgvlp lgtgndlarv lgwggsyddd |
| 301 | tqlpqilekl erastkmldr wsimtyelkl ppkasllpgp peaseefymt iyedsvathl |
| 361 | tkilnsdeha vvissaktlc etvkdfvakv ektydktlen avvadavask csvlnekleq |
| 421 | llqalhtdsq aapvlpglsp liveedaves sseeslgesk eqlgddvtkp ssqkavkpre |
| 481 | imlranslkk avrqvieeag kvmddptvhp cepanqssdy dstetdeske eakddgakes |
| 541 | itvktaprsp darasyghsq tdsvpgpava askenlpvln triicpglra glaasiagss |
| 601 | iinkmllani dpfgatpfid pdldsvdgys ekcvmnnyfg igldakisle fnnkreehpe |
| 661 | kcrsrtknlm wygvlgtrel lqrsyknleq rvqlecdgqy iplpslqgia vlnipsyagg |
| 721 | tnfwggtked difaapsfdd kilevvaifd smqmavsrvi klqhhriaqc rtvkitifgd |
| 781 | egvpvqvdge awvqppgiik ivhknraqml trdrafestl kswedkqkcd sgkpvlrthl |
| 841 | yihhaidlat eevsqmqlcs qaaeelitri cdaatihcll eqelahavna cshalnkanp |
| 901 | rcpesltrdt ateiainvka lynetesllv grvplqlesp heervsnalh svevelqklt |
| 961 | eipwlyyilh pnedeeppmd ctkrnnrstv frivpkfkke kvqkqktssq pvqkwgteev |
| 1021 | aawldllnlg eykdifirhd irgaellhle rrdlkntvge krdtkengkh mdlgipkvgh |
| 1081 | vkrilqgike lgrstpqsev |

Diacylglycerol kinase eta, isoform 4, NP_001191435.1 (SEQ ID NO: 68)

| | |
|---|---|
| 1 | mlcaenrkem edwisslksv qtrepyevaq fnvehfsgmh nwyacsharp tfcnvcresl |
| 61 | sgvtshglsc evckfkahkr cavratnnck wttlasigkd iiededgvam phqwlegnlp |
| 121 | vsakcavcdk tcgsvlrlqd wkclwcktmv htackdlyhp icplgqckvs iippialnst |
| 181 | dsdgfcratf sfcvspllvf vnsksgdnqg vkflrrfkql lnpaqvfdlm nggphlglrl |
| 241 | fqkfdnfril vcggdgsvgw vlseidklnl nkqcqlgvlp lgtgndlarv lgwggsyddd |
| 301 | tqlpqilekl erastkmldr wsimtyelkl ppkasllpgp peaseefymt iyedsvathl |
| 361 | tkilnsdeha vvissaktlc etvkdfvakv ektydktlen avvadavask csvlnekleq |
| 421 | llqalhtdsq aapvlpglsp liveedaves sseeslgesk eqlgddvtkp ssqkavkpre |
| 481 | imlranslkk avrqvieeag kvmddptvhp cepanqssdy dstetdeske eakddgakes |
| 541 | itvktaprsp darasyghsq tdsvpgpava askenlpvln triicpglra glaasiagss |
| 601 | iinkmllani dpfgatpfid pdldsvdgys ekcvmnnyfg igldakisle fnnkreehpe |
| 661 | kcrsrtknlm wygvlgtrel lqrsyknleq rvqlecdgqy iplpslqgia vinipsyagg |
| 721 | tnfwggtked difaapsfdd kilevvaifd smqmavsrvi klqhhriaqc rtvkitifgd |
| 781 | egvpvqvdge awvqppgiik ivhknraqml trdrafestl kswedkqkcd sgkpvlrthl |
| 841 | yihhaidlat eevsgmqlcs qaaeelitri cdaatihcll eqelahavna cshalnkanp |
| 901 | rcpesltrdt ateiainvka lynetesllv grvplqlesp heervsnalh svevelqklt |
| 961 | eipwlyyilh pnedeeppmd ctkrnnrstv frivpkfkke kvqkqktssq pvqkwgteev |
| 1021 | aawldllnlg eykdifirhd irgaellhle rrdlkdlgip kvghvkrilq gikelgrstp |
| 1081 | qsev |

Diacylglycerol kinase eta, isoform 5, NP_001284358.1 (SEQ ID NO: 69)

| | |
|---|---|
| 1 | mwnisqgctt gtpaptpdpp svtcaervfl esppmacpak vhtackdlyh picplgqckv |
| 61 | siippialns tdsdgfcrat fsfcvspllv fvnsksgdnq gvkflrrfkq llnpaqvfdl |
| 121 | mnggphlglr lfqkfdnfri lvcggdgsvg wvlseidkln lnkqcqlgvl plgtgndlar |
| 181 | vlgwggsydd dtqlpqilek lerastkmld rwsimtyelk lppkasllpg ppeaseefym |
| 241 | tiyedsvath ltkilnsdeh avvissaktl cetvkdfvak vektydktle navvadavas |
| 301 | kcsvlnekle qllqalhtds qaapvlpgls pliveedave ssseeslges keqlgddvtk |
| 361 | pssqkavkpr eimlranslk kavrqvieea gkvmddptvh pcepanqssd ydstetdesk |
| 421 | eeakddgake sitvktaprs pdarasyghs qtdsvpgpav aaskenlpvl ntriicpglr |
| 481 | aglaasiags siinkmllan idpfgatpfi dpdldsvdgy sekcvmnnyf gigldakisl |
| 541 | efnnkreehp ekcrsrtknl mwygvlgtre llqrsyknle qrvqlecdgq yiplpslqgi |
| 601 | avinipsyag gtnfwggtke ddifaapsfd dkilevvaif dsmqmavsrv iklqhhriaq |
| 661 | crtvkitifg degvpvqvdg eawvqppgii kivhknraqm ltrdrafest lkswedkqkc |
| 721 | dsgkpvlrth lyihhaidla teevsqmqlc sqaaeelitr icdaatihcl leqelahavn |
| 781 | acshalnkan prcpesltrd tateiainvk alynetesll vgrvplqles pheervsnal |
| 841 | hsvevelqkl teipwlyyil hpnedeeppm dctkrnnrst vfrivpkfkk ekvqkqktss |
| 901 | qpgsgdtesg sceanspgn |

Eukaryotic translation elongation factor 2, NP_001952.1 (SEQ ID NO: 70)

| | |
|---|---|
| 1 | mvnftvdqir aimdkkanir nmsviahvdh gkstltdslv ckagiiasar agetrftdtr |
| 61 | kdeqerciti kstaislfye lsendlnfik qskdgagfli nlidspghvd fssevtaalr |
| 121 | vtdgalvvvd cvsgvcvqte tvlrqaiaer ikpvlmmnkm drallelqle peelyqtfqr |
| 181 | ivenvnviis tygegesgpm gnimidpvlg tvgfgsglhg waftlkqfae myvakfaakg |
| 241 | egglgpaera kkvedmmkkl wgdryfdpan gkfsksatsp egkklprtfc qlildpifkv |
| 301 | fdaimnfkke etakliekld ikldsedkdk egkpllkavm rrwlpagdal lqmitihlps |
| 361 | pvtaqkyrce llyegppdde aamgikscdp kgplmmyisk mvptsdkgrf yafgrvfsgl |
| 421 | vstglkvrim gpnytpgkke dlylkpiqrt ilmmgryvep iedvpcgniv glvgvdqflv |
| 481 | ktgtittfeh ahnmrvmkfs vspvvrvave aknpadlpkl veglkrlaks dpmvqciiee |
| 541 | sgehiiagag elhleiclkd leedhacipi kksdpvvsyr etvseesnvl clskspnkhn |
| 601 | rlymkarpfp dglaedidkg evsarqelkq rarylaekye wdvaearkiw cfgpdgtgpn |
| 661 | iltditkgvq ylneikdsvv agfqwatkeg alceenmrgv rfdvhdvtlh adaihrgggq |
| 721 | iiptarrcly asvltaqprl mepiylveiq cpeqvvggiy gvlnrkrghv feesqvagtp |
| 781 | mfvvkaylpv nesfgftadl rsntggqafp qcvfdhwqil pgdpfdnssr psqvvaetrk |
| 841 | rkglkegipa ldnfldkl |

Eukaryotic translation initiation factor 5A, isoform A, NP_001137232.1 (SEQ
ID NO: 71)

| | |
|---|---|
| 1 | mcgtggtdsk trrpphrasf lkrleskplk maddldfetg dagasatfpm qcsalrkngf |
| 61 | vvlkgrpcki vemstsktgk hghakvhlvg idiftgkkye dicpsthnmd vpnikrndfq |
| 121 | ligiqdgyls llqdsgevre dlrlpegdlg keieqkydcg eeilitvlsa mteeaavaik |
| 181 | amak |

Eukaryotic translation initiation factor 5A, isoform B, NP_001137233.1,
NP_001137234.1, NP_001961.1 (SEQ ID NO: 72)

| | |
|---|---|
| 1 | maddldfetg dagasatfpm qcsalrkngf vvlkgrpcki vemstsktgk hghakvhlvg |
| 61 | idiftgkkye dicpsthnmd vpnikrndfq ligiqdgyls llqdsgevre dlrlpegdlg |
| 121 | keieqkydcg eeilitvlsa mteeaavaik amak |

Fibronectin 1, isoform 1 precursor, NP_997647.1 (SEQ ID NO: 73)

| | |
|---|---|
| 1 | mlrgpgpgll llavqclgta vpstgasksk rqaqqmvqpq spvaysqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipevpql tdlsfvditd ssiglrwtpl nsstiigyri tvvaagegip ifedfvdssv |
| 1321 | gyytvtglep gidydisvit linggesapt tltqqtavpp ptdlrftnig pdtmrvtwap |
| 1381 | ppsidltnfl vryspvknee dvaelsisps dnavvltnll pgteyvvsys svyeqhestp |
| 1441 | lrgrqktgld sptgidfsdi tansftvhwi apratitgyr irhhpehfsg rpredrvphs |
| 1501 | rnsitltnit pgteyvvsiv alngreespl ligqqstvsd vprdlevvaa tptslliswd |
| 1561 | apavtvryyr itygetggns pvqeftvpgs kstatisglk pgvdytitvy avtgrgdspa |
| 1621 | sskpisinyr teidkpsqmq vtdvqdnsis vkwlpssspv tgyrvtttpk ngpgptktkt |
| 1681 | agpdqtemti eglqptveyv vsvyaqnpsg esqplvqtav tnidrpkgla ftdvdvdsik |
| 1741 | iawespqgqv sryrvtyssp edgihelfpa pdgeedtael qglrpgseyt vsvvalhddm |
| 1801 | esqpligtqs taipaptdlk ftqvtptsls aqwtppnvql tgyrvrvtpk ektgpmkein |
| 1861 | lapdsssvvv sglmvatkye vsvyalkdtl tsrpaqgvvt tlenvspprr arvtdatett |
| 1921 | itiswrtkte titgfqvdav pangqtpiqr tikpdvrsyt itglqpgtdy kiylytlndn |
| 1981 | arsspvvida staidapsnl rflattpnsl lvswqpprar itgyiikyek pgspprevvp |
| 2041 | rprpgvteat itglepgtey tiyvialknn qksepligrk ktdelpqlvt lphpnlhgpe |
| 2101 | ildvpstvqk tpfvthpgyd tgngiqlpgt sgqgpsvgqq mifeehgfrr ttppttatpi |
| 2161 | rhrprpyppn vgeeiqighi predvdyhly phgpglnpna stgqealsqt tiswapfqdt |
| 2221 | seyiischpv gtdeeplqfr vpgtstsatl tgltrgatyn iivealkdqq rhkvreevvt |
| 2281 | vgnsvnegln qptddscfdp ytvshyavgd ewermsesgf kllcqclgfg sghfrcdssr |
| 2341 | wchdngvnyk igekwdrqge ngqmmsctcl gngkgefkcd pheatcyddg ktyhvgeqwq |
| 2401 | keylgaicsc tcfggqrgwr cdncrrpgge pspegttgqs ynqysqryhq rtntnvncpi |
| 2461 | ecfmpldvqa dredsre |

Fibronectin 1, isoform 3 precursor, NP_002017.1 (SEQ ID NO: 74)

| | |
|---|---|
| 1 | mlrgpgpgll llavqclgta vpstgasksk rqaqqmvqpq spvaysqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsyss vyeqhestpl rgrqktglds ptgidfsdit ansftvhwia |
| 1381 | pratitgyri rhhpehfsgr predrvphsr nsitltnitp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt nidrpkglaf tdvdvdsiki awespqgqvs ryrvtysspe dgihelfpap |
| 1681 | dgeedtaelq glrpgseytv svvalhddme sqpligtqst aipaptdlkf tqvtptslsa |
| 1741 | qwtppnvqlt gyrvrvtpke ktgpmkeinl apdsssvvvs glmvatkyev svyalkdtlt |
| 1801 | srpaqgvvtt lenvspprra rvtdatetti tiswrtktet itgfqvdavp angqtpiqrt |
| 1861 | ikpdvrsyti tglqpgtdyk iylytlndna rsspvvidas taidapsnlr flattpnsll |
| 1921 | vswqpprari tgyiikyekp gspprevvpr prpgvteati tglepgteyt iyvialknnq |
| 1981 | ksepligrkk tdelpqlvtl phpnlhgpei ldvpstvqkt pfvthpgydt gngiqlpgts |
| 2041 | gqqpsvgqqm ifeehgfrrt tppttatpir hrprpyppnv ggealsqtti swapfqdtse |
| 2101 | yiischpvgt deeplqfrvp gtstsatltg ltrgatynii vealkdqqrh kvreevvtvg |
| 2161 | nsvneglnqp tddscfdpyt vshyavgdew ermsesgfkl lcqclgfgsg hfrcdssrwc |
| 2221 | hdngvnykig ekwdrqgeng qmmsctclgn gkgefkcdph eatcyddgkt yhvgeqwqke |
| 2281 | ylgaicsctc fggqrgwrcd ncrrpggeps pegttgqsyn gysqryhqrt ntnvncpiec |
| 2341 | fmpldvqadr edsre |

Fibronectin 1, isoform 4 precursor, NP_997643.1 (SEQ ID NO: 75)

| | |
|---|---|
| 1 | mlrgpgpgll llavqclgta vpstgasksk rqaqqmvqpq spvaysqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsyss vyeqhestpl rgrqktglds ptgidfsdit ansftvhwia |
| 1381 | pratitgyri rhhpehfsgr predrvphsr nsitltnitp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt nidrpkglaf tdvdvdsiki awespqgqvs ryrvtysspe dgihelfpap |
| 1681 | dgeedtaelq glrpgseytv svvalhddme sqpligtqst aipaptdlkf tqvtptslsa |
| 1741 | qwtppnvqlt gyrvrvtpke ktgpmkeinl apdsssvvvs glmvatkyev svyalkdtlt |
| 1801 | srpaqgvvtt lenvspprra rvtdatetti tiswrtktet itgfqvdavp angqtpiqrt |
| 1861 | ikpdvrsyti tglqpgtdyk iylytlndna rsspvvidas taidapsnlr flattpnsll |
| 1921 | vswqpprari tgyiikyekp gspprevvpr prpgvteati tglepgteyt iyvialknnq |
| 1981 | ksepligrkk tvqktpfvth pgydtgngiq lpgtsgqqps vgqqmifeeh gfrrttpptt |
| 2041 | atpirhrprp yppnvgqeal sqttiswapf qdtseyiisc hpvgtdeepl qfrvpgtsts |
| 2101 | atltgltrga tyniivealk dqqrhkvree vvtvgnsvne glnqptddsc fdpytvshya |
| 2161 | vgdewermse sgfkllcqcl gfgsghfrcd ssrwchdngv nykigekwdr qgengqmmsc |
| 2221 | tclgngkgef kcdpheatcy ddgktyhvge qwqkeylgai csctcfggqr gwrcdncrrp |
| 2281 | ggepspegtt ggsynqysqr yhqrtntnvn cpiecfmpld vqadredsre |

Fibronectin 1, isoform 5 precursor, NP_997641.1 (SEQ ID NO: 76)

| | |
|---|---|
| 1 | mlrgpgpgll llavqclgta vpstgasksk rqaqqmvqpq spvaysqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsyss vyeqhestpl rgrqktglds ptgidfsdit ansftvhwia |
| 1381 | pratitgyri rhhpehfsgr predrvphsr nsitltnitp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyagnpsge |
| 1621 | sqplvqtavt tipaptdlkf tqvtptslsa qwtppnvqlt gyrvrvtpke ktgpmkeinl |
| 1681 | apdsssvvvs glmvatkyev svyalkdtlt srpaqgvvtt lenvspprra rvtdatetti |
| 1741 | tiswrtktet itgfqvdavp angqtpiqrt ikpdvrsyti tglqpgtdyk iylytlndna |
| 1801 | rsspvvidas taidapsnlr flattpnsll vswqpprari tgyiikyekp gspprevvpr |
| 1861 | prpgvteati tglepgteyt iyvialknnq ksepligrkk tdelpqlvtl phpnlhgpei |
| 1921 | ldvpstvqkt pfvthpgydt gngiqlpgts gqqpsvgqqm ifeehgfrrt tppttatpir |
| 1981 | hrprpyppnv geeiqighip redvdyhlyp hgpglnpnas tgqealsqtt iswapfqdts |
| 2041 | eyiischpvg tdeeplqfrv pgtstsatlt gltrgatyni ivealkdqqr hkvreevvtv |
| 2101 | gnsvneglnq ptddscfdpy tvshyavgde wermsesgfk llcqclgfgs ghfrcdssrw |
| 2161 | chdngvnyki gekwdrqgen gqmmsctclg ngkgefkcdp heatcyddgk tyhvgeqwqk |
| 2221 | eylgaicsct cfggqrgwrc dncrrpggep spegttgqsy nqysqryhqr tntnvncpie |
| 2281 | cfmpldvqad redsre |

Fibronectin 1, isoform 6 precursor, NP_997639.1 (SEQ ID NO: 77)

| | |
|---|---|
| 1 | mlrgpgpgll llavqclgta vpstgasksk rqaqqmvqpq spvaysqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsyss vyeqhestpl rgrqktglds ptgidfsdit ansftvhwia |
| 1381 | pratitgyri rhhpehfsgr predrvphsr nsitltnitp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt tipaptdlkf tqvtptslsa qwtppnvqlt gyrvrvtpke ktgpmkeinl |
| 1681 | apdsssvvvs glmvatkyev svyalkdtlt srpaqgvvtt lenvspprra rvtdatetti |
| 1741 | tiswrtktet itgfqvdavp angqtpiqrt ikpdvrsyti tglqpgtdyk iylytlndna |
| 1801 | rsspvvidas taidapsnlr flattpnsll vswqpprari tgyiikyekp gspprevvpr |
| 1861 | prpgvteati tglepgteyt iyvialknnq ksepligrkk tgqealsqtt iswapfqdts |
| 1921 | eyiischpvg tdeeplqfrv pgtstsatlt gltrgatyni ivealkdqqr hkvreevvtv |
| 1981 | gnsvneglnq ptddscfdpy tvshyavgde wermsesgfk llcqclgfgs ghfrcdssrw |
| 2041 | chdngvnyki gekwdrqgen gqmmsctclg ngkgefkcdp heatcyddgk tyhvgeqwqk |
| 2101 | eylgaicsct cfggqrgwrc dncrrpggep spegttgqsy nqysqryhqr tntnvncpie |
| 2161 | cfmpldvqad redsre |

Fibronectin 1, isoform 7 precursor, NP_473375.2 (SEQ ID NO: 78)

| | |
|---|---|
| 1 | mlrgpgpgll llavqclgta vpstgasksk rqaqqmvqpq spvaysgskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpvsi pprnlgy |

Fibronectin 1, isoform 8 precursor, NP_001293058.1 (SEQ ID NO: 79)

| | |
|---|---|
| 1 | mlrgpgpgll llavqclgta vpstgasksk rqaqqmvqpq spvaysqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipevpql tdlsfvditd ssiglrwtpl nsstiigyri tvvaagegip ifedfvdssv |
| 1321 | gyytvtglep gidydisvit linggesapt tltqqtavpp ptdlrftnig pdtmrvtwap |
| 1381 | ppsidltnfl vryspvknee dvaelsisps dnavvltnll pgteyvvsys svyeqhestp |
| 1441 | lrgrqktgld sptgidfsdi tansftvhwi apratitgyr irhhpehfsg rpredrvphs |
| 1501 | rnsitltnit pgteyvvsiv alngreespl ligqqstvsd vprdlevvaa tptslliswd |
| 1561 | apavtvryyr itygetggns pvqeftvpgs kstatisglk pgvdytitvy avtgrgdspa |
| 1621 | sskpisinyr teidkpsqmq vtdvqdnsis vkwlpssspv tgyrvtttpk ngpgptktkt |
| 1681 | agpdqtemti eglqptveyv vsvyaqnpsg esqplvqtav tnidrpkgla ftdvdvdsik |
| 1741 | iawespqgqv sryrvtyssp edgihelfpa pdgeedtael qglrpgseyt vsvvalhddm |
| 1801 | esqpligtqs taipaptdlk ftqvtptsls aqwtppnvql tgyrvrvtpk ektgpmkein |
| 1861 | lapdsssvvv sglmvatkye vsvyalkdtl tsrpaqgvvt tlenvspprr arvtdatett |
| 1921 | itiswrtkte titgfqvdav pangqtpiqr tikpdvrsyt itglqpgtdy kiylytlndn |
| 1981 | arsspvvida staidapsnl rflattpnsl lvswqpprar itgyiikyek pgspprevvp |
| 2041 | rprpgvteat itglepgtey tiyvialknn qksepligrk ktdelpqlvt lphpnlhgpe |
| 2101 | ildvpstvqk tpfvthpgyd tgngiqlpgt sgqgpsvgqq mifeehgfrr ttppttatpi |
| 2161 | rhrprpyppn vgqealsqtt iswapfqdts eyiischpvg tdeeplqfrv pgtstsatlt |
| 2221 | gltrgatyni ivealkdqqr hkvreevvtv gnsvneglnq ptddscfdpy tvshyavgde |
| 2281 | wermsesgfk llcqclgfgs ghfrcdssrw chdngvnyki gekwdrqgen gqmmsctclg |
| 2341 | ngkgefkcdp heatcyddgk tyhvgeqwqk eylgaicsct cfggqrgwrc dncrrpggep |
| 2401 | spegttgqsy nqysqryhqr tntnvncpie cfmpldvqad redsre |

Fibronectin 1, isoform 9 precursor, NP_001293059.1 (SEQ ID NO: 80)

| | |
|---|---|
| 1 | mlrgpgpgll llavqclgta vpstgasksk rqaqqmvqpq spvaysqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipevpql tdlsfvditd ssiglrwtpl nsstiigyri tvvaagegip ifedfvdssv |
| 1321 | gyytvtglep gidydisvit linggesapt tltqqtavpp ptdlrftnig pdtmrvtwap |
| 1381 | ppsidltnfl vryspvknee dvaelsisps dnavvltnll pgteyvvsys svyeqhestp |
| 1441 | lrgrqktgld sptgidfsdi tansftvhwi apratitgyr irhhpehfsg rpredrvphs |
| 1501 | rnsitltnit pgteyvvsiv alngreespl ligqqstvsd vprdlevvaa tptslliswd |
| 1561 | apavtvryyr itygetggns pvqeftvpgs kstatisglk pgvdytitvy avtgrgdspa |
| 1621 | sskpisinyr teidkpsqmq vtdvqdnsis vkwlpssspv tgyrvtttpk ngpgptktkt |
| 1681 | agpdqtemti eglqptveyv vsvyaqnpsg esqplvqtav ttipaptdlk ftqvtptsls |
| 1741 | aqwtppnvql tgyrvrvtpk ektgpmkein lapdsssvvv sglmvatkye vsvyalkdtl |
| 1801 | tsrpaqgvvt tlenvspprr arvtdatett itiswrtkte titgfqvdav pangqtpiqr |
| 1861 | tikpdvrsyt itglqpgtdy kiylytlndn arsspvvida staidapsnl rflattpnsl |
| 1921 | lvswqpprar itgyiikyek pgspprevvp rprpgvteat itglepgtey tiyvialknn |
| 1981 | qksepligrk ktgqealsqt tiswapfqdt seyiischpv gtdeeplqfr vpgtstsatl |
| 2041 | tgltrgatyn iivealkdqq rhkvreevvt vgnsvnegln qptddscfdp ytvshyavgd |
| 2101 | ewermsesgf kllcqclgfg sghfrcdssr wchdngvnyk igekwdrqge ngqmmsctcl |
| 2161 | gngkgefkcd pheatcyddg ktyhvgeqwq keylgaicsc tcfggqrgwr cdncrrpgge |
| 2221 | pspegttgqs ynqysqryhq rtntnvncpi ecfmpldvqa dredsre |

Fibronectin 1, isoform 10 precursor, NP_001293060.1 (SEQ ID NO: 81)

| | |
|---|---|
| 1 | mlrgpgpgll llavqclgta vpstgasksk rqaqqmvqpq spvavsqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsyss vyeqhestpl rgrqktglds ptgidfsdit ansftvhwia |
| 1381 | pratitgyri rhhpehfsgr predrvphsr nsitltnltp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt tipaptdlkf tqvtptslsa qwtppnvqlt gyrvrvtpke ktgpmkeinl |
| 1681 | apdsssvvvs glmvatkyev svyalkdtlt srpaqgvvtt lenvspprra rvtdatetti |
| 1741 | tiswrtktet itgfqvdavp angqtpiqrt ikpdvrsyti tglqpgtdyk iylytlndna |
| 1801 | rsspvvidas taidapsnlr flattpnsll vswqpprari tgyiikyekp gspprevvpr |
| 1861 | prpgvteati tglepgteyt iyvialknnq ksepligrkk tdelpqlvtl phpnlhgpei |
| 1921 | ldvpstvqkt pfvthpgydt gngiqlpgts gqqpsvgqqm ifeehgfrrt tppttatpir |
| 1981 | hrprpyppnv ggealsqtti swapfqdtse yiischpvgt deeplqfrvp gtstsatltg |
| 2041 | ltrgatynii vealkdqqrh kvreevvtvg nsvneglnqp tddscfdpyt vshyavgdew |
| 2101 | ermsesgfkl lcgclgfgsg hfrcdssrwc hdngvnykig ekwdrqgeng qmmsctclgn |
| 2161 | gkgefkcdph eatcyddgkt yhvgeqwqke ylgaicsctc fggqrgwrcd ncrrpggeps |
| 2221 | pegttgqsyn gysqryhqrt ntnvncpiec fmpldvqadr edsre |

Fibronectin 1, isoform 11 precursor, NP_001293061.1 (SEQ ID NO: 82)

| | |
|---|---|
| 1 | mlrgpgpgll llavqclgta vpstgasksk rqaqqmvqpq spvaysqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsvsk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsyss vyeqhestpl rgrqktglds ptgidfsdit ansftvhwia |
| 1381 | pratitgyri rhhpehfsgr predrvphsr nsitltnitp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt tipaptdlkf tqvtptslsa qwtppnvqlt gyrvrvtpke ktgpmkeinl |
| 1681 | apdsssvvvs glmvatkyev svyalkdtlt srpaqgvvtt lenvspprra rvtdatetti |
| 1741 | tiswrtktet itgfqvdavp angqtpiqrt ikpdvrsyti tglqpgtdyk iylytlndna |
| 1801 | rsspvvidas taidapsnlr flattpnsll vswqpprari tgyiikyekp gspprevvpr |
| 1861 | prpgvteati tglepgteyt iyvialknnq ksepligrkk tvqktpfvth pgydtgngiq |
| 1921 | lpgtsgqqps vgqqmifeeh gfrrttpptt atpirhrprp yppnvggeal sqttiswapf |
| 1981 | qdtseyiisc hpvgtdeepl qfrvpgtsts atltgltrga tyniivealk dqqrhkvree |
| 2041 | vvtvgnsvne glnqptddsc fdpytvshya vgdewermse sgfkllcgcl gfgsghfrcd |
| 2101 | ssrwchdngv nykigekwdr qgengqmmsc tclgngkgef kcdpheatcy ddgktyhvge |
| 2161 | qwgkeylgai csctcfggqr gwrcdncrrp ggepspegtt gqsynqysqr yhqrtntnvn |
| 2221 | cpiecfmpld vqadredsre |

Major histocompatibility complex, class II, DR beta 1, precursor,
NP_001230894.1 (SEQ ID NO: 83)

| | |
|---|---|
| 1 | mvclrlpggs cmavltvtlm vlssplalag dtrprfleys tsechffngt ervryldryf |
| 61 | hnqeenvrfd sdvgefravt elgrpdaeyw nsqkdlleqk rgrvdnycrh nygvvesftv |
| 121 | qrrvhpkvtv ypsktqplqh hnllvcsvsg fypgsievrw frngqeektg vvstglihng |
| 181 | dwtfqtlvml etvprsgevy tcqvehpsvt spltvewrar sesaqskmls gvggfvlgll |
| 241 | flgaglfiyf rnqkghsglq prgfls |

Major histocompatibility complex, class II, DR beta 1, precursor,
NP_001346122.1 (SEQ ID NO: 84)

| | |
|---|---|
| 1 | mvclklpggs cmaaltvtlm vlssplalag dtqprflwqg kykchffngt ervqflerlf |
| 61 | ynqeefvrfd sdvgeyravt elgrpvaesw nsqkdiledr rgqvdtvcrh nygvgesftv |
| 121 | qrrvhpevtv ypaktqplqh hnllvcsvsg fypgsievrw frngqeekag vvstgliqng |
| 181 | dwtfqtlvml etvprsgevy tcqvehpsvm spltvewrar sesaqskmls gvggfvlgll |
| 241 | flgaglfiyf rnqkghsglq ptgfls |

Major histocompatibility complex, class II, DR beta 1, precursor,
NP_001346123.1 (SEQ ID NO: 85)

| | |
|---|---|
| 1 | mvclkfpggs cmaaltvtlm vlssplalag dtrprfleqv khechffngt ervrfldryf |
| 61 | yhqeeyvrfd sdvgeyravt elgrpdaeyw nsqkdlleqr raevdtycrh nygvvesftv |
| 121 | qrrvypevtv ypaktqplqh hnllvcsvng fypgsievrw frngqeektg vvstgliqng |
| 181 | dwtfqtlvml etvprsgevy tcqvehpslt spltvewrar sesaqskmls gvggfvlgll |
| 241 | flgaglfiyf rnqkghsglq ptgfls |

Major histocompatibility complex, class II, DR beta 1, precursor, NP_002115.2
(SEQ ID NO: 86)

| | |
|---|---|
| 1 | mvclklpggs cmtaltvtlm vlssplalsg dtrprflwqp krechffngt ervrfldryf |
| 61 | ynqeesvrfd sdvgefravt elgrpdaeyw nsqkdileqa raavdtycrh nygvvesftv |
| 121 | qrrvqpkvtv ypsktqplqh hnllvcsysg fypgsievrw flngqeekag mvstgliqng |
| 181 | dwtfqtlvml etvprsgevy tcqvehpsvt spltvewrar sesaqskmls gvggfvlgll |
| 241 | flgaglfiyf rnqkghsglq ptgfls |

Major histocompatibility complex, class II, DR beta 5, precursor, NP_002116.2
(SEQ ID NO: 87)

| | |
|---|---|
| 1 | mvclklpggs ymakltvtlm vlssplalag dtrprflqqd kyechffngt ervrflhrdi |
| 61 | ynqeedlrfd sdvgeyravt elgrpdaeyw nsqkdfledr raavdtycrh nygvgesftv |
| 121 | qrrvepkvtv ypartqtlqh hnllvcsvng fypgsievrw frnsqeekag vvstgliqng |
| 181 | dwtfqtlvml etvprsgevy tcqvehpsvt spltvewraq sesagskmls gvggfvlgll |
| 241 | flgaglfiyf knqkghsglh ptglvs |

Hydroxysteroid 17-beta dehydrogenase 3, NP_000188.1 (SEQ ID NO: 88)

| | |
|---|---|
| 1 | mgdvleqffi ltgllvclac lakcvrfsrc vllnywkvlp ksflrsmgqw avitgagdgi |
| 61 | gkaysfelak rglnvvlisr tlekleaiat eierttgrsv kiiqadftkd diyehikekl |
| 121 | agleigilvn nvgmlpnllp shflnapdei qslihcnits vvkmtqlilk hmesrqkgli |
| 181 | lnissgialf pwplysmysa skafvcafsk alqeeykake viiqvltpya vstamtkyln |
| 241 | tnvitktade fvkeslnyvt iggetcgcla heilagflsl ipawafysga fqrlllthyv |
| 301 | aylklntkvr |

Insulin degrading enzyme, isoform 1, NP_004960.2 (SEQ ID NO: 89)

| | |
|---|---|
| 1 | mryrlawllh palpstfrsv lgarlppper lcgfqkktys kmnnpaikri gnhitksped |
| 61 | kreyrglela ngikvllisd pttdkssaal dvhigslsdp pniaglshfc ehmlflgtkk |
| 121 | ypkeneysqf lsehagssna ftsgehtnyy fdvshehleg aldrfaqffl cplfdesckd |
| 181 | revnavdseh eknvmndawr lfqlekatgn pkhpfskfgt gnkytletrp nqegidvrqe |
| 241 | llkfhsayys snlmavcvlg reslddltnl vvklfseven knvplpefpe hpfqeehlkg |
| 301 | lykivpikdi rnlyvtfpip dlqkyyksnp ghylghligh egpgsllsel kskgwvntiv |
| 361 | ggqkegargf mffiinvdlt eegllhvedi ilhmfqyiqk lraegpqewv fqeckdlnav |
| 421 | afrfkdkerp rgytskiagi lhyypleevl taeylleefr pdliemvldk lrpenvrvai |
| 481 | vsksfegktd rteewygtqy kqeaipdevi kkwqnadlng kfklptknef iptnfeilpl |
| 541 | ekeatpypal ikdtamsklw fkqddkfflp kaclnfeffs pfayvdplhc nmaylylell |
| 601 | kdslneyaya aelaglsydl qntiygmyls vkgyndkqpi llkkiiekma tfeidekrfe |
| 661 | iikeaymrsl nnfraeqphq hamyylrllm tevawtkdel kealddvtlp rlkafipqll |
| 721 | srlhieallh gnitkqaalg imqmvedtli ehahtkpllp sqlvryrevq lpdrgwfvyq |
| 781 | qrnevhnncg ieiyyqtdmq stsenmflel fcqiisepcf ntlrtkeqlg yivfsgprra |
| 841 | ngiqglrfii qsekpphyle srveaflitm eksiedmtee afqkhiqala irrldkpkkl |
| 901 | saecakywge iisqqynfdr dntevaylkt ltkediikfy kemlavdapr rhkvsvhvla |
| 961 | remdscpvvg efpcqndinl sqapalpqpe viqnmtefkr glplfplvkp hinfmaakl |

Insulin degrading enzyme, isoform 2, NP_001159418.1 (SEQ ID NO: 90)

| | |
|---|---|
| 1 | msklwfkqdd kfflpkacln feffspfayv dplhcnmayl ylellkdsln eyayaaelag |
| 61 | lsydlqntiy gmylsvkgyn dkqpillkki iekmatfeid ekrfeiikea ymrslnnfra |
| 121 | eqphqhamyy lrllmtevaw tkdelkeald dvtlprlkaf ipqllsrlhi eallhgnitk |
| 181 | qaalgimqmv edtliehaht kpllpsqlvr yrevqlpdrg wfvyqqrnev hnncgieiyy |
| 241 | qtdmqstsen mflelfcqii sepcfntlrt keqlgyivfs gprrangiqg lrfiiqsekp |
| 301 | phylesrvea flitmeksie dmteeafqkh iqalairrld kpkklsaeca kywgeiisqq |
| 361 | ynfdrdntev aylktltked iikfykemla vdaprrhkvs vhvlaremds cpvvgefpcq |
| 421 | ndinlsqapa lpqpeviqnm tefkrglplf plvkphinfm aakl |

Insulin degrading enzyme, isoform 3, NP_001309722.1 (SEQ ID NO: 91)

| | |
|---|---|
| 1 | mryrlawllh palpstfrsv lgarlppper lcgfqkktys kmnnpaikri gnhitksped |
| 61 | kreyrglela ngikvllisd pttdkssaal dvhigslsdp pniaglshfc ehmlflgtkk |
| 121 | ypkeneysqf lsehagssna ftsgehtnyy fdvshehleg aldrfaqffl cplfdesckd |
| 181 | revnavdseh eknvmndawr lfqlekatgn pkhpfskfgt gnkytletrp nqegidvrqe |
| 241 | llkfhsayys snlmavcvlg reslddltnl vvklfseven knvplpefpe hpfqeehlkq |
| 301 | lykivpikdi rnlyvtfpip dlqkyyksnp ghylghligh egpgsllsel kskgwvntlv |
| 361 | ggqkegargf mffiinvdlt eegllhvedi ilhmfqyiqk lraegpqewv fqeckdlnav |
| 421 | afrfkdkerp rgytskiagi lhyypleevl taeylleefr pdliemvldk lrpenvrvai |
| 481 | vsksfegktd rteewygtqy kqeaipdevi kkwqnadlng kfklptknef iptnfeilpl |
| 541 | ekeatpypal ikdtamsklw fkqddkfflp kaclnfeffs ryiyadplhc nmtylfirll |
| 601 | kddlkeytya arlsglsygi asgmnaills vkgyndkqpi llkkiiekma tfeidekrfe |
| 661 | iikeaymrsl nnfraeqphq hamyylrllm tevawtkdel kealddvtlp rlkafipqll |
| 721 | srlhieallh gnitkqaalg imqmvedtli ehahtkpllp sqlvryrevq lpdrgwfvyq |
| 781 | qrnevhnncg ieiyyqtdmq stsenmflel fcqiisepcf ntlrtkeqlg yivfsgprra |
| 841 | ngiqglrfii qsekpphyle srveaflitm eksiedmtee afqkhiqala irrldkpkkl |
| 901 | saecakywge iisqqynfdr dntevaylkt ltkediikfy kemlavdapr rhkvsvhvla |
| 961 | remdscpvvg efpcqndinl sqapalpqpe viqnmtefkr glplfplvkp hinfmaakl |

Insulin degrading enzyme, isoform 4, NP_001309723.1 (SEQ ID NO: 92)

| | |
|---|---|
| 1 | mryrlawllh palpstfrsv lgarlppper lcgfqkktys kmnnpaikri gnhitksped |
| 61 | kreyrglela ngikvllisd pttdkssaal dvhigslsdp pniaglshfc ehmlflgtkk |
| 121 | ypkeneysqf lsehagssna ftsgehtnyy fdvshehleg aldrfaqffl cplfdesckd |
| 181 | revnavdseh eknvmndawr lfqlekatgn pkhpfskfgt greslddltn lvvklfseve |
| 241 | nknvplpefp ehpfqeehlk qlykivpikd irnlyvtfpi pdlqkyyksn pghylghlig |
| 301 | hegpgsllse lkskgwvntl vggqkegarg fmffiinvdl teegllhved iilhmfqyiq |
| 361 | klraegpqew vfqeckdlna vafrfkdker prgytskiag ilhyypleev ltaeylleef |
| 421 | rpdliemvld klrpenvrva ivsksfegkt drteewygtq ykqeaipdev ikkwqnadln |
| 481 | gkfklptkne fiptnfeilp lekeatpypa likdtamskl wfkqddkffl pkaclnfeff |
| 541 | spfayvdplh cnmaylylel lkdslneyay aaelaglsyd lqntiygmyl svkgyndkqp |
| 601 | illkkiiekm atfeidekrf eiikeaymrs lnnfraeqph qhamyylrll mtevawtkde |
| 661 | lkealddvtl prlkafipql lsrlhieall hgnitkqaal gimqmvedtl iehahtkpll |
| 721 | psqlvryrev qlpdrgwfvy qqrnevhnnc gieiyyqtdm qstsenmfle lfcqiisepc |
| 781 | fntlrtkeql gyivfsgprr angiqglrfi iqsekpphyl esrveaflit meksiedmte |
| 841 | eafqkhiqal airrldkpkk lsaecakywg eiisqqynfd rdntevaylk tltkediikf |
| 901 | ykemlavdap rrhkvsvhvl aremdscpvv gefpcqndin lsqapalpqp eviqnmtefk |
| 961 | rglplfplvk phinfmaakl |

Insulin degrading enzyme, isoform 5, NP_001309724.1, NP_001309725.1 (SEQ ID
NO: 93)

| | |
|---|---|
| 1 | mnnpaikrig nhitkspedk reyrglelan gikvllisdp ttdkssaald vhigslsdpp |
| 61 | niaglshfce hmlflgtkky pkeneysqfl sehagssnaf tsgehtnyyf dvshehlega |
| 121 | ldrfaqfflc plfdesckdr evnavdsehe knvmndawrl fqlekatgnp khpfskfgtg |
| 181 | nkytletrpn qegidvrqel lkfhsayyss nlmavcvlgr eslddltnlv vklfsevenk |
| 241 | nvplpefpeh pfqeehlkql ykivpikdir nlyvtfpipd lqkyyksnpg hylghlighe |
| 301 | gpgsllselk skgwvntlvg gqkegargfm ffiinvdlte egllhvedii lhmfqyiqkl |
| 361 | raegpqewvf qeckdlnava frfkdkerpr gytskiagil hyypleevlt aeylleefrp |
| 421 | dliemvldkl rpenvrvaiv sksfegktdr teewygtqyk qeaipdevik kwqnadlngk |
| 481 | fklptknefi ptnfeilple keatpypali kdtamsklwf kqddkfflpk aclnfeffsp |
| 541 | fayvdplhcn maylylellk dslneyayaa elaglsydlq ntiygmylsv kgyndkqpil |
| 601 | lkkiiekmat feidekrfei ikeaymrsln nfraeqphqh amyylrllmt evawtkdelk |
| 661 | ealddvtlpr lkafipqlls rlhieallhg nitkqaalgi mqmvedtlie hahtkpllps |
| 721 | qlvryrevql pdrgwfvyqq rnevhnncgi eiyyqtdmqs tsenmflelf cqiisepcfn |
| 781 | tlrtkeqlgy ivfsgprran gigqlrfiiq sekpphyles rveaflitme ksiedmteea |
| 841 | fqkhiqalai rrldkpkkls aecakywgei isqqynfdrd ntevaylktl tkediikfyk |
| 901 | emlavdaprr hkvsvhvlar emdscpvvge fpcqndinls qapalpqpev iqnmtefkrg |
| 961 | lplfplvkph infmaakl |

Insulin degrading enzyme, isoform 6, NP_001309726.1 (SEQ ID NO: 94)

| | |
|---|---|
| 1 | msklwfkqdd kfflpkacln feffsryiya dplhcnmtyl firllkddlk eytyaarlsg |
| 61 | lsygiasgmn aillsvkgyn dkqpillkki iekmatfeid ekrfeiikea ymrslnnfra |
| 121 | eqphqhamyy lrllmtevaw tkdelkeald dvtlprlkaf ipqllsrlhi eallhgnitk |
| 181 | qaalgimqmv edtliehaht kpllpsqlvr yrevqlpdrg wfvyqqrnev hnncgieiyy |
| 241 | qtdmqstsen mflelfcqii sepcfntlrt keqlgyivfs gprrangiqg lrfiiqsekp |
| 301 | phylesrvea flitmeksie dmteeafqkh iqalairrld kpkklsaeca kywgeiisqq |
| 361 | ynfdrdntev aylktltked iikfykemla vdaprrhkvs vhvlaremds cpvvgefpcq |
| 421 | ndinlsqapa lpqpeviqnm tefkrglplf plvkphinfm aakl |

Indoleamine 2,3-dioxygenase 1, NP_002155.1 (SEQ ID NO: 95)

| | |
|---|---|
| 1 | mahamenswt iskeyhidee vgfalpnpqe nlpdfyndwm fiakhlpdli esgqlrerve |
| 61 | klnmlsidhl tdhksqrlar lvlgcitmay vwgkghgdvr kvlprniavp ycqlskklel |
| 121 | ppilvyadcv lanwkkkdpn kpltyenmdv lfsfrdgdcs kgfflvsllv eiaaasaikv |
| 181 | iptvfkamqm qerdtllkal leiascleka lqvfhqihdh vnpkaffsvl riylsgwkgn |
| 241 | pqlsdglvye gfwedpkefa ggsagqssvf qcfdvllgiq qtaggghaaq flqdmrrymp |
| 301 | pahrnflcsl esnpsvrefv lskgdaglre aydacvkalv slrsyhlqiv tkyilipasq |
| 361 | qpkenktsed pskleakgtg gtdlmnflkt vrstteksll keg |

Insulin like growth factor binding protein 5, precursor, NP_000590.1 (SEQ ID
NO: 96)

| | |
|---|---|
| 1 | mvlltavlll laayagpaqs lgsfvhcepc dekalsmcpp splgcelvke pgcgccmtca |
| 61 | laegqscgvy tercaqglrc lprqdeekpl hallhgrgvc lneksyreqv kierdsrehe |
| 121 | epttsemaee tyspkifrpk htriselkae avkkdrrkkl tqskfvggae ntahpriisa |
| 181 | pemrqeseqg porrhmeasl qelkasprmv pravylpncd rkgfykrkqc kpsrgrkrgi |
| 241 | cwcvdkygmk lpgmeyvdgd fqchtfdssn ve |

Insulin like growth factor binding protein 7, isoform 1 precursor,
NP_001544.1 (SEQ ID NO: 97)

| | |
|---|---|
| 1 | merpslrall lgaaglllll lplssssssd tcgpcepasc pplpplgcll getrdacgcc |
| 61 | pmcargegep cggggagrgy capgmecvks rkrrkgkaga aaggpgvsgv cvcksrypvc |
| 121 | gsdgttypsg cqlraasqra esrgekaitq vskgtceqgp sivtppkdiw nvtgaqvyls |
| 181 | cevigiptpv liwnkvkrgh ygvqrtellp gdrdnlaiqt rggpekhevt gwvlvsplsk |
| 241 | edageyecha snsqgqasas akitvvdalh eipvkkgega el |

Insulin like growth factor binding protein 7, isoform 2 precursor,
NP_001240764.1 (SEQ ID NO: 98)

| | |
|---|---|
| 1 | merpslrall lgaaglllll lplssssssd tcgpcepasc pplpplgcll getrdacgcc |
| 61 | pmcargegep cggggagrgy capgmecvks rkrrkgkaga aaggpgvsgv cvcksrypvc |
| 121 | gsdgttypsg cqlraasqra esrgekaitq vskgtceqgp sivtppkdiw nvtgaqvyls |
| 181 | cevigiptpv liwnkvkrgh ygvqrtellp gdrdnlaiqt rggpekhevt gwvlvsplsk |
| 241 | edageyecha snsqgqasas akitvvdalh eipvkkgtq |

Potassium two pore domain channel subfamily K member 1, NP_002236.1 (SEQ ID
NO: 99)

| | |
|---|---|
| 1 | mlqslagssc vrlverhrsa wcfgflvlgy llylvfgavv fssvelpyed llrqelrklk |
| 61 | rrfleehecl seqqleqflg rvleasnygv svlsnasgnw nwdftsalff astvlsttgy |
| 121 | ghtvplsdgg kafciiysvi gipftllflt avvqritvhv trrpvlyfhi rwgfskqvva |
| 181 | ivhavllgfv tvscfffipa avfsvleddw nflesfyfcf islstiglgd yvpgegynqk |
| 241 | frelykigit cylllgliam lvvletfcel helkkfrkmf yvkkdkdedq vhiiehdqls |
| 301 | fssitdqaag mkedqkqnep fvatqssacv dgpanh |

Lysosomal associated membrane protein 3, precursor, NP_055213.2 (SEQ ID NO:
100)

| | |
|---|---|
| 1 | mprqlsaaaa lfaslavilh dgsqmrakaf petrdysqpt aaatvqdikk pvqqpakqap |
| 61 | hqtlaarfmd ghitfqtaat vkiptttpat tkntattspi tytlvttqat pnnshtappv |
| 121 | tevtvgpsla pyslpptitp pahttgtsss tvshttgntt qpsnqttlpa tlsialhkst |
| 181 | tgqkpvqpth apgttaaahn ttrtaapast vpgptlapqp ssvktgiyqv lngsrlcika |
| 241 | emgiqlivqd kesvfsprry fnidpnatqa sgncgtrksn lllnfqggfv nitftkdees |
| 301 | yyisevgayl tvsdpetiyq gikhavvmfq tavghsfkcv seqslqlsah lqvkttdvql |
| 361 | qafdfeddhf gnvdecssdy tivlpvigai vvglclmgmg vykirlrcqs sgyqri |

MAGE family member B2, NP_002355.2 (SEQ ID NO: 101)

| | |
|---|---|
| 1 | mprgqksklr arekrrkard etrglnvpqv teaeeeeapc csssvsggaa ssspaagipq |
| 61 | epqrapttaa aaaagvsstk skkgakshqg eknasssqas tstkspsedp ltrksgslvq |
| 121 | fllykykikk svtkgemlki vgkrfrehfp eilkkasegl svvfglelnk vnpnghtytf |
| 181 | idkvdltdee sllsswdfpr rkllmpllgv iflngnsate eeiweflnml gvydgeehsv |
| 241 | fgepwklitk dlvqekyley kqvpssdppr fqflwgpray aetskmkvle flakvngttp |
| 301 | cafpthyeea lkdeekagv |

Mitogen-activated protein kinase 13, NP_002745.1 (SEQ ID NO: 102)

| | |
|---|---|
| 1 | mslirkkgfy kqdvnktawe lpktyvspth vgsgaygsvc saidkrsgek vaikklsrpf |
| 61 | qseifakray rellllkhmq henviglldv ftpasslrnf ydfylvmpfm qtdlqkimgm |
| 121 | efseekiqyl vyqmlkglky ihsagvvhrd lkpgnlavne dcelkildfg larhadaemt |
| 181 | gyvvtrwyra pevilswmhy nqtvdiwsvg cimaemltgk tlfkgkdyld qltqilkvtg |
| 241 | vpgtefvqkl ndkaaksyiq slpqtprkdf tqlfpraspq aadllekmle ldvdkrltaa |
| 301 | qalthpffep frdpeeetea qqpfddsleh ekltvdewkq hiykeivnfs piarkdsrrr |
| 361 | sgmkl |

Macrophage receptor with collagenous structure, NP_006761.1 (SEQ ID NO: 103)

| | |
|---|---|
| 1 | mrnkkilked ellsetqqaa fhqiamepfe invpkpkrrn gvnfslavvv iylilltaga |
| 61 | gllvvqvinl qarlrvlemy flndtlaaed spsfsllqsa hpgehlaqga srlqvlqaql |
| 121 | twvrvshehl lqrvdnftqn pgmfrikgeq gapglqghkg amgmpgapgp pgppaekgak |
| 181 | gamgrdgatg psgpqgppgv kgeaglqgpq gapgkqgatg tpgpqgekgs kgdggligpk |
| 241 | getgtkgekg dlglpgskgd rgmkgdagvm gppgaqgskg dfgrpgppgl agfpgakgdq |
| 301 | gqpglqgvpg ppgavghpga kgepgsagsp graglpgspg spgatglkgs kgdtglqgqq |
| 361 | grkgesgvpg pagvkgeqgs pglagpkgap ggagqkgdqg vkgssgeqgv kgekgergen |
| 421 | svsvrivgss nrgraevyys gtwgticdde wqnsdaivfc rmlgyskgra lykvgagtgq |
| 481 | iwldnvqcrg testlwsctk nswghhdcsh eedagvecsv |

Malic enzyme 1, NADP-dependent malic enzyme, NP_002386.1 (SEQ ID NO: 104)

| | |
|---|---|
| 1 | mepeaprrrh thqrgylltr nphlnkdlaf tleerqqlni hgllppsfns qeiqvlrvvk |
| 61 | nfehlnsdfd rylllmdlqd rneklfyrvl tsdiekfmpi vytptvglac qqyslvfrkp |
| 121 | rglfitihdr ghiasvinaw pedvikaivv tdgerilglg dlgcngmgip vgklalytac |
| 181 | ggmnpqeclp vildvgtene ellkdplyig lrqrrvrgse yddfldefme aysskygmnc |
| 241 | liqfedfanv nafrllnkyr nqyctfnddi qgtasvavag llaalritkn klsdqtilfq |
| 301 | gageaalgia hlivmaleke glpkekaikk iwlvdskgli vkgrasltqe kekfahehee |
| 361 | mknleaivqe ikptaligva aiggafseqi lkdmaafner piifalsnpt skaecsaeqc |
| 421 | ykitkgraif asgspfdpvt lpngqtlypg qgnnsyvfpg valgvvacgl rqitdniflt |
| 481 | taeviaqqvs dkhleegrly ppintirdvs lkiaekivkd ayqektatvy pepqnkeafv |
| 541 | rsqmystdyd qilpdcyswp eevqkiqtkv dq |

Migration and invasion inhibitory protein, NP_068752.2 (SEQ ID NO: 105)

| | |
|---|---|
| 1 | mveaeelaql rllnlellrq lwvggdavrr svaraasess lessssynse tpstpetsst |
| 61 | slstscprgr ssvwgppdac rgdlrdvars gvaslppakc qhqeslgrpr phsapslgts |
| 121 | slrdpepsgr lgdpgpqeaq tprsilaqqs klskprvtfs eesavpkrsw rlrpylgydw |
| 181 | iagsldtsss itsgpeaffs klqefretnk eecicshpep qlpglressg sgveedhecv |
| 241 | ycyrvnrrlf pvpvdpgtpc rlcrtprdqq gpgtlaqpah vrvsiplsil epphryhihr |
| 301 | rksfdasdtl alprhcllgw difppkseks saprnldlws svsaeaqhqk lsgtsspfhp |
| 361 | aspmqmlppt ptwsvpqvpr phvprqkp |

Matrix metallopeptidase 12, macrophage metalloelastase preproprotein,
NP_002417.2 (SEQ ID NO: 106)

| | |
|---|---|
| 1 | mkfllilllq atasgalpin sstsleknnv lfgerylekf ygleinklpv tkmkysgnlm |
| 61 | kekiqemqhf lglkvtgqld tstlemmhap rcgvpdvhhf rempggpvwr khyityrinn |
| 121 | ytpdmnredv dyairkafqv wsnvtplkfs kintgmadil vvfargahgd fhafdgkggi |
| 181 | lahafgpgsg iggdahfded efwtthsggt nlfltavhei ghslglghss dpkavmfpty |
| 241 | kyvdintfrl saddirgiqs lygdpkenqr lpnpdnsepa lcdpnlsfda vttvgnkiff |
| 301 | fkdrffwlkv serpktsvnl isslwptlps gieaayeiea rnqvflfkdd kywlisnlrp |
| 361 | epnypksihs fgfpnfvkki daavfnprfy rtyffvdnqy wryderrqmm dpgypklitk |
| 421 | nfqgigpkid avfysknkyy yffqgsnqfe ydfllqritk tlksnswfgc |

Matrix metallopeptidase 7, matrilysin preproprotein, NP_002414.1 (SEQ ID NO:
107)

| | |
|---|---|
| 1 | mrltvlcavc llpgslalpl pqeaggmsel qwegaqdylk rfylydsetk nansleaklk |
| 61 | emqkffglpi tgmlnsrvie imqkprcgvp dvaeyslfpn spkwtskvvt yrivsytrdl |
| 121 | phitvdrlvs kalnmwgkei plhfrkvvwg tadimigfar gahgdsypfd gpgntlahaf |
| 181 | apgtglggda hfdederwtd gsslginfly aathelghsl gmghssdpna vmyptygngd |
| 241 | pqnfklsqdd ikgiqklygk rsnsrkk |

Myelin protein zero like 1, myelin protein zero-like protein 1 isoform a
precursor, NP_003944.1 (SEQ ID NO: 108)

| | |
|---|---|
| 1 | maasagagav iaapdsrrwl wsvlaaalgl ltagvsalev ytpkeifvan gtqgkltckf |
| 61 | kststtgglt syswsfqpeg adttvsffhy sqgqvylgny ppfkdriswa gdldkkdasi |
| 121 | nienmqfihn gtyicdvknp pdivvqpghi rlyvvekenl pvfpvwvvvg ivtavvlglt |
| 181 | llismilavl yrrknskrdy tgcstsesls pvkqaprksp sdteglvksl psgshqgpvi |
| 241 | yaqldhsggh hsdkinkses vvyadirkn |

Myelin protein zero like 1, myelin protein zero-like protein 1 isoform b
precursor, NP_078845.3 (SEQ ID NO: 109)

| | |
|---|---|
| 1 | maasagagav iaapdsrrwl wsvlaaalgl ltagvsalev ytpkeifvan gtqgkltckf |
| 61 | kststtgglt syswsfqpeg adttvsffhy sqgqvylgny ppfkdriswa gdldkkdasi |
| 121 | nienmqfihn gtyicdvknp pdivvqpghi rlyvvekenl pvfpvwvvvg ivtavvlglt |
| 181 | llismilavl yrrknskrdy tgaqsymhs |

Myelin protein zero like 1, myelin protein zero-like protein 1 isoform c
precursor, NP_001139663.1 (SEQ ID NO: 110)

| | |
|---|---|
| 1 | maasagagav iaapdsrrwl wsvlaaalgl ltagvsalev ytpkeifvan gtqgkltckf |
| 61 | kststtgglt syswsfqpeg adttvsgpvi yaqldhsggh hsdkinkses vvyadirkn |

Macrophage scavenger receptor 1, macrophage scavenger receptor types I and II
isoform type 1, NP_619729.1 (SEQ ID NO: lll)

| | |
|---|---|
| 1 | meqwdhfhnq qedtdscses vkfdarsmta llppnpknsp slqeklksfk aalialyllv |
| 61 | favlipligi vaaqllkwet kncsysstna nditqsltgk gndseeemrf qevfmehmsn |
| 121 | mekriqhild meanlmdteh fqnfsmttdq rfndillqls tlfssvqghg naideisksl |
| 181 | islnttlldl qlnienlngk iqentfkqqe eiskleervy nvsaeimamk eeqvhleqei |
| 241 | kgevkvlnni tndlrlkdwe hsqtlrnitl iqgppgppge kgdrgptges gprgfpgpig |
| 301 | ppglkgdrga igfpgsrglp gyagrpgnsg pkgqkgekgs gntltpftkv rlvggsgphe |
| 361 | grveilhsgq wgticddrwe vrvgqvvcrs lgypgvqavh kaahfgqgtg piwlnevfcf |
| 421 | gressieeck irqwgtracs hsedagvtct l |

Macrophage scavenger receptor 1, macrophage scavenger receptor types I and II
isoform type 2, NP_002436.1 (SEQ ID NO: 112)

| | |
|---|---|
| 1 | meqwdhfhnq qedtdscses vkfdarsmta llppnpknsp slqeklksfk aalialyllv |
| 61 | favlipligi vaaqllkwet kncsvsstna nditqsltgk gndseeemrf qevfmehmsn |
| 121 | mekriqhild meanlmdteh fqnfsmttdq rfndillqls tlfssvqghg naideisksl |
| 181 | islnttlldl qlnienlngk iqentfkqqe eiskleervy nvsaeimamk eeqvhleqei |
| 241 | kgevkvlnni tndlrlkdwe hsqtlrnitl iqgppgppge kgdrgptges gprgfpgpig |
| 301 | ppglkgdrga igfpgsrglp gyagrpgnsg pkgqkgekgs gntlrpvqlt dhiragps |

Macrophage scavenger receptor 1, macrophage scavenger receptor types I and II
isoform type 3, NP_619730.1 (SEQ ID NO: 113)

| | |
|---|---|
| 1 | meqwdhfhnq qedtdscses vkfdarsmta llppnpknsp slqeklksfk aalialyllv |
| 61 | favlipligi vaaqllkwet kncsysstna nditqsltgk gndseeemrf qevfmehmsn |
| 121 | mekriqhild meanlmdteh fqnfsmttdq rfndillqls tlfssvqghg naideisksl |
| 181 | islnttlldl qlnienlngk iqentfkqqe eiskleervy nvsaeimamk eeqvhleqei |
| 241 | kgevkvlnni tndlrlkdwe hsqtlrnitl iqgppgppge kgdrgptges gprgfpgpig |
| 301 | ppglkgdrga igfpgsrglp gyagrpgnsg pkgqkgekgs gntlstgpiw lnevfcfgre |
| 361 | ssieeckirq wgtracshse dagvtctl |

Myoneurin, isoform A, NP_001172047.1, NP_061127.1 (SEQ ID NO: 114)

| | |
|---|---|
| 1 | mqyshhcehl lerinkqrea gflcdctivi gefqfkahrn vlasfseyfg aiyrstsenn |
| 61 | vfldqsqvka dgfqkllefi ytgtlnldsw nvkeihqaad ylkveevvtk ckikmedfaf |
| 121 | ianpssteis sitgnielnq qtclltlrdy nnreksevst dliganpkqg alakkssqtk |
| 181 | kkkkafnspk tgqnktvqyp sdilenasve lfldanklpt pvveqvaqin dnseleltsv |
| 241 | ventfpaqdi vhtvtvkrkr gksqpncalk ehsmsniasv kspyeaensg eeldqryska |
| 301 | kpmcntcgkv fseasslrrh mrihkgvkpy vchlcgkaft qcnqlkthvr thtgekpykc |
| 361 | elcdkgfaqk cqlvfhsrmh hgeekpykcd vcnlqfatss nlkiharkhs gekpyvcdrc |
| 421 | gqrfaqastl tyhvrrhtge kpyvcdtcgk afayssslit hsrkhtgekp yicgicgksf |
| 481 | issgelnkhf rshtgerpfi celcgnsytd iknlkkhktk vhsgadktld ssaedhtlse |
| 541 | qdsiqkspls etmdvkpsdm tlplalplgt edhhmllpvt dtqsptsdtl lrstvngyse |
| 601 | pqliflqqly |

Myoneurin, isoform B, NP_001172048.1 (SEQ ID NO: 115)

| | |
|---|---|
| 1 | mqyshhcehl lerinkqrea gflcdctivi gefqfkahrn vlasfseyfg aiyrstsenn |
| 61 | vfldqsqvka dgfqkllefi ytgtlnldsw nvkeihqaad ylkveevvtk ckikmedfaf |
| 121 | ianpssteis sitgnielnq qtclltlrdy nnreksevst dliqanpkqg alakkssqtk |
| 181 | kkkkafnspk tgqnktvqyp sdilenasve lfldanklpt pvveqvaqin dnseleltsv |
| 241 | ventfpaqdi vhtvtvkrkr gksqpncalk ehsmsniasv kspyeaensg eeldqryska |
| 301 | kpmcntcgkv fseasslrrh mrihkgvkpy vchlcgkaft qcnqlkthvr thtgekpykc |
| 361 | elcdkgfaqk cqlvfhsrmh hgeekpykcd vcnlqfatss nlkiharkhs gekpyvcdrc |
| 421 | gqrfaqastl tyhvrrhtge kpyvcdtcgk afayssslit hsrkhtgekp yicgicgksf |
| 481 | issgelnkhf rshtgadktl dssaedhtls eqdsiqkspl setmdvkpsd mtlplalplg |
| 541 | tedhhmllpv tdtqsptsdt llrstvngys epqliflqql y |

N-acetylglucosamine kinase, isoform 1, NP_060037.3 (SEQ ID NO: 116)

| | |
|---|---|
| 1 | mrtrtgsqla arevtgsgav prqlegrrcq agrdanggts sdgsssmaai yggvegggtr |
| 61 | sevllvsedg kilaeadgls tnhwligtdk cverinemvn rakrkagvdp lvplrslgls |
| 121 | lsggdqedag rilieelrdr fpylsesyli ttdaagsiat atpdggvvli sgtgsncrli |
| 181 | npdgsesgcg gwghmmgdeg saywiahqav kivfdsidnl eaaphdigyv kqamfhyfqv |
| 241 | pdrlgilthl yrdfdkcrfa gfcrkiaega qqgdplsryi frkagemlgr hivavlpeid |
| 301 | pvlfqgkigl pilcvgsvwk swellkegfl laltqgreiq aqnffssftl mklrhssalg |
| 361 | gaslgarhig hllpmdysan aiafysytfs |

N-acetylglucosamine kinase, isoform 2, NP_001317354.1, NP_001317355.1 (SEQ ID
NO: 117)

| | |
|---|---|
| 1 | mvnrakrkag vdplvplrsl glslsggdqe dagrilieel rdrfpylses ylittdaags |
| 61 | iatatpdggv vlisgtgsnc rlinpdgses gcggwghmmg degsaywiah qavkivfdsi |
| 121 | dnleaaphdi gyvkqamfhy fqvpdrlgil thlyrdfdkc rfagfcrkia egaqqgdpls |
| 181 | ryifrkagem lgrhivavlp eidpvlfqgk iglpilcvgs vwkswellke gfllaltqgr |
| 241 | eiqaqnffss ftlmklrhss alggaslgar highllpmdy sanaiafysy tfs |

Napsin A aspartic peptidase, preproprotein, NP_004842.1 (SEQ ID NO: 118)

| | |
|---|---|
| 1 | mspppllqpl llllpllnve psgatlirip lhrvqpgrri lnllrgwrep aelpklgaps |
| 61 | pgdkpifvpl snyrdvqyfg eiglgtppqn ftvafdtgss nlwvpsrrch ffsvpcwlhh |
| 121 | rfdpkasssf qangtkfaiq ygtgrvdgil sedkltiggi kgasvifgea lwepslvfaf |
| 181 | ahfdgilglg fpilsvegvr ppmdvlveqg lldkpvfsfy lnrdpeepdg gelvlggsdp |
| 241 | ahyippltfv pvtvpaywqi hmervkvgpg ltlcakgcaa ildtgtslit gpteeiralh |
| 301 | aaiggiplla geyiilcsei pklpavsfll ggvwfnitah dyviqttrng vrlclsgfqa |
| 361 | ldvpppagpf wilgdvflgt yvavfdrgdm kssarvglar artrgadlgw getaqaqfpg |

Nuclear transcription factor Y subunit gamma, isoform 1, NP_001136060.1 (SEQ
ID NO: 119)

| | |
|---|---|
| 1 | msteggfggt sssdaqqslq sfwprvmeei rnitvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvqgq qqgqqttsst ttiqpgqiii |
| 181 | aqpqqgqttp vtmqvgegqq vqivqaqpqg qaqqaqsgtg qtmqvmqqii tntgeiqqip |
| 241 | vqlnagqlqy irlaqpvsgt qvvqgqiqtl atnaqqgqrn asqgkprrcl ketlqitqte |
| 301 | vqqgqqqfsq ftdgqqlyqi qqvtmpagqd laqpmfiqsa nqpsdgqapq vtgd |

Nuclear transcription factor Y subunit gamma, isoform 2, NP_055038.2 (SEQ ID
NO: 120)

| | |
|---|---|
| 1 | msteggfggt sssdaqqslq sfwprvmeei rnltvkdfry qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvggq qqgqqttsst ttiqpgqiii |
| 181 | aqpqqgqttp vtmqvgeggq qgivqaqpqg qaqqaqsgtg qtmqvmqqii tntgeiqqip |
| 241 | vqlnagqlqy irlaqpvsgt qvvqgqiqtl atnaqqitqt evqqgqqqfs qftdgqqlyq |
| 301 | iqqvtmpagq dlaqpmfiqs anqpsdgqap qvtgd |

Nuclear transcription factor Y subunit gamma, isoform 3, NP_001136059.1 (SEQ
ID NO: 121)

| | |
|---|---|
| 1 | msteggfggt sssdaqqslq sfwprvmeei rnltvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvggq qqgqqttsst ttiqpgqiii |
| 181 | aqpqqgqttp vtmqvgegqq vqivqaqpqg qaqqaqsgtg qtmqvmqqii tntgeiqqip |
| 241 | vqlnagqlqy irlaqpvsgt qvvqgqiqtl atnaqqitqt evqqgqqqfs qftdgqlyqi |
| 301 | qqvtmpagqd laqpmfiqsa nqpsdgqapq vtgd |

Nuclear transcription factor Y subunit gamma, isoform 4, NP_001136061.1 (SEQ
ID NO: 122)

| | |
|---|---|
| 1 | msteggfggt sssdaqqslq sfwprvmeei rnltvkdfrv qelplarikk imkldedvkr |
| 61 | ndiamaitkf dqfdflidiv prdelkppkr qeevrqsvtp aepvqyyftl aqqptavqvq |
| 121 | gqqqgqqtts stttiqpgqi iiaqpqqgqt tpvtmqvgeg qqvqivqaqp qgqaqqaqsg |
| 181 | tgqtmqvmqq iitntgeigq ipvqlnagql gyirlaqpvs gtqvvqgqiq tlatnaqqit |
| 241 | qtevqqgqqq fsqftdgqql yqiqqvtmpa gqdlaqpmfi qsanqpsdgq apqvtgd |

Nuclear transcription factor Y subunit gamma, isoform 5, NP_001136062.1 (SEQ
ID NO: 123)

| | |
|---|---|
| 1 | msteggfggt sssdaqqslq sfwprvmeei rnltvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvggq qqgqqttsst ttiqpgqiii |
| 181 | aqpqqgqtmq vmqqiitntg eiqqipvqln agqlqyirla qpvsgtqvvg gqiqtlatna |
| 241 | qgitqtevqq gqqqfsqftd gqqlyqiqqv tmpagqdlaq pmfiqsanqp sdgqapqvtg |
| 301 | d |

Nuclear transcription factor Y subunit gamma, isoform 6, NP_001295043.1 (SEQ
ID NO: 124)

| | |
|---|---|
| 1 | msteggfggt sssdaqqslq sfwprvmeei rnltvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvggq qqgqqttsst ttiqpgqiii |
| 181 | aqpqqgqttp vtmqvgegqq vqivqaqpqg qaqqaqsgtg qtmqvmqqii tntgeiqqip |
| 241 | vqlnagqlqy irlaqpvsgt qvvqgqiqtl atnaqqgqrn asqgkprrcl ketlqitqte |
| 301 | vqqgqqqfsq ftdgqrnsvg qarvseltge aeprevkatg nstpctsslp tthppshrag |
| 361 | ascvccsqpq qsstspppsd alqwvvvevs gtpnqlethr elhaplpgmt slsplhpsqq |
| 421 | lyqiqqvtmp agqdlaqpmf iqsanqpsdg qapqvtgd |

Nuclear transcription factor Y subunit gamma, isoform 7, NP_001295044.1 (SEQ
ID NO: 125)

| | |
|---|---|
| 1 | msteggfggt sssdaqqslq sfwprvmeei rnltvkdfrv qelplarikk imkldedvkm |
| 61 | isaeapvlfa kaaqifitel tlrawihted nkrrtlqrnd iamaitkfdq fdflidivpr |
| 121 | delkppkrqe evrqsvtpae pvqyyftlaq qptavqvggq qqgqqttsst ttiqpgqiii |
| 181 | aqpqqgqttp vtmqvgeggq vqivqaqpqg qaqqaqsgtg qtmqvmqqii tntgeiqqip |
| 241 | vqlnagqlqy irlaqpvsgt qvvqgqiqtl atnaqqitqt evqqgqqqfs qftdgqrnsv |
| 301 | qqarvseltg eaeprevkat gnstpctssl ptthppshra gascvccsqp qqsstsppps |
| 361 | dalqwvvvev sgtpnqleth relhaplpgm tslsplhpsq qlyqiqqvtm pagqdlaqpm |
| 421 | fiqsanqpsd gqapqvtgd |

NFKB repressing factor, isoform 1, NP_001166958.1 (SEQ ID NO: 126)

| | |
|---|---|
| 1 | mgfmlplifr ysprlmekil qmaegidige mpsydlvlsk pskgqkrhls tcdgqnppkk |
| 61 | qagskfharp rfepvhfvas sskderqedp ygpqtkevne qthfasmprd iyqdytqdsf |
| 121 | siqdgnsqyc dssgfiltkd qpvtanmyfd sgnpapstts qqansqstpe pspsqtfpes |
| 181 | vvaekqyfie kltatiwknl snpemtsgsd kinytymltr ciqacktnpe yiyaplkeip |
| 241 | padipknkkl ltdgyacevr cqniylttgy agskngsrdr atelavkllq krievrvvrr |
| 301 | kfkhtfgedl vvcqigmssy efppalkppe dlvvlgkdas gqpifnasak hwtnfviten |
| 361 | andaigilnn sasfnkmsie ykyemmpnrt wrcrvflqdh claegygtkk tskhaaadea |
| 421 | lkilqktqpt ypsvkssqch tgssprgsgk kkdikdlvvy enssnpvctl ndtaqfnrmt |
| 481 | veyvyermtg lrwkckvile seviaeavgv kktvkyeaag eavktlkktq ptvinnlkkg |
| 541 | avedvisrne iqgrsaeeay kqqikednig nqllrkmgwt ggglgksgeg irepisvkeq |
| 601 | hkreglgldv ervnkiakrd ieqiirnyar seshtdltfs reltnderkq ihqiaqkygl |
| 661 | kskshgvghd rylvvgrkrr kedlldqlkq egqvghyelv mpqan |

NFKB repressing factor, isoform 2, NP_001166959.1, NP_060014.2 (SEQ ID NO:
127)

| | |
|---|---|
| 1 | mekilqmaeg idigempsyd lvlskpskgq krhlstcdgq nppkkqagsk fharprfepv |
| 61 | hfvassskde rqedpygpqt kevneqthfa smprdiyqdy tqdsfsiqdg nsqycdssgf |
| 121 | iltkdqpvta nmyfdsgnpa psttsqqans qstpepspsq tfpesvvaek qyfiekltat |
| 181 | iwknlsnpem tsgsdkinyt ymltrciqac ktnpeyiyap lkeippadip knkklltdgy |
| 241 | acevrcqniy lttgyagskn gsrdratela vkllqkriev rvvrrkfkht fgedlvvcqi |
| 301 | gmssyefppa lkppedlvvl gkdasgqpif nasakhwtnf vitenandai gilnnsasfn |
| 361 | kmsieykyem mpnrtwrcrv flqdhclaeg ygtkktskha aadealkilq ktqptypsvk |
| 421 | ssqchtgssp rgsgkkkdik dlvvyenssn pvctlndtaq fnrmtveyvy ermtglrwkc |
| 481 | kvilesevia eavgvkktvk yeaageavkt lkktqptvin nlkkgavedv isrneiqgrs |
| 541 | aeeaykqqik ednignqllr kmgwtggglg ksgegirepi svkeqhkreg lgldvervnk |
| 601 | iakrdieqii rnyarsesht dltfsreltn derkqihqia qkyglksksh gvghdrylvv |
| 661 | grkrrkedll dqlkqegqvg hyelvmpqan |

Plasminogen activator, urokinase, urokinase-type plasminogen activator
isoform 1 preproprotein, NP_002649.1 (SEQ ID NO: 128)

| | |
|---|---|
| 1 | mrallarlll cvlvvsdskg snelhqvpsn cdclnggtcv snkyfsnihw cncpkkfggq |
| 61 | hceidksktc yegnghfyrg kastdtmgrp clpwnsatvl qqtyhahrsd alqlglgkhn |
| 121 | ycrnpdnrrr pwcyvqvglk plvqecmvhd cadgkkpssp peelkfqcgq ktlrprfkii |
| 181 | ggefttienq pwfaaiyrrh rggsvtyvcg gslispcwvi sathcfidyp kkedyivylg |
| 241 | rsrinsntqg emkfevenli lhkdysadtl ahhndiallk irskegrcaq psrtiqticl |
| 301 | psmyndpqfg tsceitgfgk enstdylype qlkmtvvkli shrecqqphy ygsevttkml |
| 361 | caadpqwktd scqgdsggpl vcslqgrmtl tgivswgrgc alkdkpgvyt rvshflpwir |
| 421 | shtkeengla l |

Plasminogen activator, urokinase, urokinase-type plasminogen activator
isoform 2, NP_001138503.1 (SEQ ID NO: 129)

| | |
|---|---|
| 1 | mvfhlrtrye qancdclngg tcvsnkyfsn ihwcncpkkf ggqhceidks ktcyegnghf |
| 61 | yrgkastdtm grpclpwnsa tvlqqtyhah rsdalqlglg khnycrnpdn rrrpwcyvqv |
| 121 | glkplvqecm vhdcadgkkp ssppeelkfq cgqktlrprf kiiggeftti enqpwfaaiy |
| 181 | rrhrggsvty vcggslispc wvisathcfi dypkkedyiv ylgrsrinsn tqgemkfeve |
| 241 | nlilhkdysa dtlahhndia llkirskegr caqpsrtiqt iclpsmyndp qfgtsceitg |
| 301 | fgkenstdyl ypeqlkmtvv klishrecqq phyygsevtt kmlcaadpqw ktdscqgdsg |
| 361 | gplvcslqgr mtltgivswg rgcalkdkpg vytrvshflp wirshtkeen glal |

Plasminogen activator, urokinase, urokinase-type plasminogen activator
isoform 3, NP_001306120.1 (SEQ ID NO: 130)

| | |
|---|---|
| 1 | mgrpclpwns atvlqqtyha hrsdalqlgl gkhnycrnpd nrrrpwcyvq vglkplvqec |
| 61 | mvhdcadgkk pssppeelkf qcgqktlrpr fkiiggeftt ienqpwfaai yrrhrggsvt |
| 121 | yvcggslisp cwvisathcf idypkkedyi vylgrsrlns ntqgemkfev enlilhkdys |
| 181 | adtlahhndi allkirskeg rcaqpsrtiq ticlpsmynd pqfgtsceit gfgkenstdy |
| 241 | lypeqlkmtv vklishrecq qphyygsevt tkmlcaadpq wktdscqgds ggplvcslqg |
| 301 | rmtltgivsw grgcalkdkp gvytrvshfl pwirshtkee nglal |

Receptor tyrosine kinase like orphan receptor 1, inactive tyrosine-protein
kinase transmembrane receptor ROR1 isoform 1 precursor, NP_005003.2 (SEQ ID
NO: 131)

| | |
|---|---|
| 1 | mhrprrrgtr ppllallaal llaargaaaq etelsvsael vptsswniss elnkdsyltl |
| 61 | depmnnitts lgqtaelhck vsgnppptir wfkndapvvq eprrlsfrst iygsrlrirn |
| 121 | ldttdtgyfq cvatngkevv sstgvlfvkf gppptaspgy sdeyeedgfc qpyrgiacar |
| 181 | fignrtvyme slhmqgeien qitaaftmig tsshlsdkcs qfaipslchy afpycdetss |
| 241 | vpkprdlcrd eceilenvlc qteyifarsn pmilmrlklp ncedlpqpes peaancirig |
| 301 | ipmadpinkn hkcynstgvd yrgtvsvtks grqcqpwnsq yphthtftal rfpelngghs |
| 361 | ycrnpgnqke apwcftlden fksdlcdipa cdskdskekn kmeilyilvp svaiplaial |
| 421 | lffficvcrn nqksssapvq rqpkhvrgqn vemsmlnayk pkskakelpl savrfmeelg |
| 481 | ecafgkiykg hlylpgmdha qlvaiktlkd ynnpqqwtef qqeaslmael hhpnivcllg |
| 541 | avtqeqpvcm lfeyinqgdl heflimrsph sdvgcssded gtvkssldhg dflhiaiqia |
| 601 | agmeylsshf fvhkdlaarn iligeqlhvk isdlglsrei ysadyyrvqs ksllpirwmp |
| 661 | peaimygkfs sdsdiwsfgv vlweifsfgl qpyygfsnqe viemvrkrql lpcsedcppr |
| 721 | myslmtecwn eipsrrprfk dihvrlrswe glsshtsstt psggnattqt tslsaspvsn |
| 781 | lsnprypnym fpsqgitpqg qiagfigppi pqnqrfipin gypippgyaa fpaahygptg |
| 841 | pprviqhcpp pksrspssas gststghvts lpssgsnqea nipllphmsi pnhpggmgit |
| 901 | vfgnksqkpy kidskqasll gdanihghte smisael |

Receptor tyrosine kinase like orphan receptor 1, inactive tyrosine-protein
kinase transmembrane receptor ROR1 isoform 2 precursor, NP_001077061.1 (SEQ
ID NO: 132)

| | |
|---|---|
| 1 | mhrprrrgtr ppllallaal llaargaaaq etelsvsael vptsswniss elnkdsyltl |
| 61 | depmnnitts lgqtaelhck vsgnppptir wfkndapvvq eprrlsfrst iygsrlrirn |
| 121 | ldttdtgyfq cvatngkevv sstgvlfvkf gppptaspgy sdeyeedgfc qpyrgiacar |
| 181 | fignrtvyme slhmqgeien qitaaftmig tsshlsdkcs qfaipslchy afpycdetss |
| 241 | vpkprdlcrd eceilenvlc qteyifarsn pmilmrlklp ncedlpqpes peaancirig |
| 301 | ipmadpinkn hkcynstgvd yrgtvsvtks grqcqpwnsq yphthtftal rfpelngghs |
| 361 | yornpgngke apwcftlden fksdlcdipa cgk |

Runt related transcription factor 1, runt-related transcription factor 1
isoform AML1a, NP_001116079.1 (SEQ ID NO: 133)

| | |
|---|---|
| 1 | mripvdasts rrftppstal spgkmsealp lgapdagaal agklrsgdrs mvevladhpg |
| 61 | elvrtdspnf lcsvlpthwr cnktlpiafk vvalgdvpdg tlvtvmagnd enysaelrna |
| 121 | taamknqvar fndlrfvgrs grgksftlti tvftnppqva tyhraikitv dgpreprrhr |
| 181 | qklddqtkpg slsfserlse leqlrrtamr vsphhpaptp npraslnhst afnpqpqsqm |
| 241 | qeedtapwrc |

Runt related transcription factor 1, runt-related transcription factor 1
isoform AML1b, NP_001001890.1 (SEQ ID NO: 134)

| | |
|---|---|
| 1 | mripvdasts rrftppstal spgkmsealp lgapdagaal agklrsgdrs mvevladhpg |
| 61 | elvrtdspnf lcsvlpthwr cnktlpiafk vvalgdvpdg tivtvmagnd enysaelrna |
| 121 | taamknqvar fndlrfvgrs grgksftlti tvftnppqva tyhraikitv dgpreprrhr |
| 181 | qklddqtkpg slsfserlse leqlrrtamr vsphhpaptp npraslnhst afnpqpqsqm |
| 241 | qdtrqiqpsp pwsydqsyqy lgsiaspsvh patpispgra sgmttlsael ssrlstapdl |
| 301 | tafsdprqfp alpsisdprm hypgaftysp tpvtsgigig msamgsatry htylpppypg |
| 361 | ssqaqggpfq asspsyhlyy gasagsyqfs mvggersppr ilppctnast gsallnpslp |
| 421 | nqsdvveaeg shsnsptnma psarleeavw rpy |

Runt related transcription factor 1, runt-related transcription factor 1
isoform AML1c, NP_001745.2 (SEQ ID NO: 135)

| | |
|---|---|
| 1 | masdsifesf psypqcfmre cilgmnpsrd vhdastsrrf tppstalspg kmsealplga |
| 61 | pdagaalagk lrsgdrsmve vladhpgelv rtdspnflcs vlpthwrcnk tlpiafkvva |
| 121 | lgdvpdgtlv tvmagndeny saelrnataa mknqvarfnd lrfvgrsgrg ksftltitvf |
| 181 | tnppqvatyh raikitvdgp reprrhrqkl ddqtkpgsls fserlseleq lrrtamrvsp |
| 241 | hhpaptpnpr aslnhstafn pqpqsqmqdt rqiqpsppws ydqsyqylgs iaspsvhpat |
| 301 | pispgrasgm ttlsaelssr lstapdltaf sdprqfpalp sisdprmhyp gaftysptpv |
| 361 | tsgigigmsa mgsatryhty lpppypgssq aqggpfqass psyhlyygas agsyqfsmvg |
| 421 | gerspprilp pctnastgsa llnpslpnqs dvveaegshs nsptnmapsa rleeavwrpy |

Surfactant protein A1, pulmonary surfactant-associated protein Al isoform 1
precursor, NP_001158116.1, NP_001158119.1, NP_005402.3 (SEQ ID NO: 136)

| | |
|---|---|
| 1 | mwlcplalnl ilmaasgavc evkdvcvgsp gipgtpgshg lpgrdgrdgl kgdpgppgpm |
| 61 | gppgempcpp gndglpgapg ipgecgekge pgergppglp ahldeelqat lhdfrhqilq |
| 121 | trgalslqgs imtvgekvfs sngqsitfda iqeacaragg riavprnpee neaiasfvkk |
| 181 | yntyayvglt egpspgdfry sdgtpvnytn wyrgepagrg keqcvemytd gqwndrncly |
| 241 | srlticef |

Surfactant protein A1, pulmonary surfactant-associated protein Al isoform 2
precursor, NP_001087239.2 (SEQ ID NO: 137)

| | |
|---|---|
| 1 | mrpcqvpgaa tgpramwlcp lalnlilmaa sgavcevkdv cvgspgipgt pgshglpgrd |
| 61 | grdglkgdpg ppgpmgppge mpcppgndgl pgapgipgec gekgepgerg ppglpahlde |
| 121 | elqatlhdfr hqilqtrgal slqgsimtvg ekvfssngqs itfdaiqeac araggriavp |
| 181 | rnpeeneaia sfvkkyntya yvgltegpsp gdfrysdgtp vnytnwyrge pagrgkeqcv |
| 241 | emytdgqwnd rnclysrlti cef |

Surfactant protein A1, pulmonary surfactant-associated protein Al isoform 3
precursor, NP_001158117.1 (SEQ ID NO: 138)

| | |
|---|---|
| 1 | mrpcqvpgaa tgpramwlcp lalnlilmaa sgavcevkdv cvgtpgipge cgekgepger |
| 61 | gppglpahld eelqatlhdf rhqilqtrga lslqgsimtv gekvfssngq sitfdaiqea |
| 121 | caraggriav prnpeeneai asfvkkynty ayvgltegps pgdfrysdgt pvnytnwyrg |
| 181 | epagrgkeqc vemytdgqwn drnclysrlt icef |

Surfactant protein A1, pulmonary surfactant-associated protein Al isoform 4
precursor, NP_001158118.1 (SEQ ID NO: 139)

| | |
|---|---|
| 1 | mwlcplalnl ilmaasgavc evkdvcvgtp gipgecgekg epgergppgl pahldeelqa |
| 61 | tlhdfrhqil qtrgalslqg simtvgekvf ssngqsitfd aiqeacarag griavprnpe |
| 121 | eneaiasfvk kyntyayvgl tegpspgdfr ysdgtpvnyt nwyrgepagr gkeqcvemyt |
| 181 | dgqwndrncl ysrlticef |

Surfactant protein A2, pulmonary surfactant-associated protein A2 isoform 1
precursor, NP_001092138.1, NP_001307742.1 (SEQ ID NO: 140)

| | |
|---|---|
| 1 | mwlcplaltl ilmaasgaac evkdvcvgsp gipgtpgshg lpgrdgrdgv kgdpgppgpm |
| 61 | gppgetpcpp gnnglpgapg vpgergekge agergppglp ahldeelqat lhdfrhqilq |
| 121 | trgalslqgs imtvgekvfs sngqsitfda iqeacaragg riavprnpee neaiasfvkk |
| 181 | yntyayvglt egpspgdfry sdgtpvnytn wyrgepagrg keqcvemytd gqwndrncly |
| 241 | srlticef |

Surfactant protein A2, pulmonary surfactant-associated protein A2 isoform 2
precursor, NP_001307743.1 (SEQ ID NO: 141)

| | |
|---|---|
| 1 | mpgaatgpra mwlcplaltl ilmaasgaac evkdvcvgsp gipgtpgshg lpgrdgrdgv |
| 61 | kgdpgppgpm gppgetpcpp gnnglpgapg vpgergekge agergppglp ahldeelqat |
| 121 | lhdfrhqilq trgalslqgs imtvgekvfs sngqsitfda iqeacaragg riavprnpee |
| 181 | neaiasfvkk yntyayvglt egpspgdfry sdgtpvnytn wyrgepagrg keqcvemytd |
| 241 | gqwndrncly srlticef |

Surfactant protein B, pulmonary surfactant-associated protein B precursor,
NP_000533.3, NP_942140.2 (SEQ ID NO: 142)

| | |
|---|---|
| 1 | mhqagypgcr gamaeshllq wlllllptic gpgtaawtts slacaqgpef wcgsleqalq |
| 61 | cralghclqe vwghvgaddl cqecedivhi lnkmakeaif qdtmrkfleq ecnvlplkll |
| 121 | mpqcnqvldd yfplvidyfq nqtdsngicm hlglcksrqp epeqepgmsd plpkplrdpl |
| 181 | pdplldklvl pvlpgalqar pgphtqdlse qqfpiplpyc wlcralikri qamipkgala |
| 241 | vavaqvcrvv plvaggicqc laerysvill dtllgrmlpq lvcrlvlrcs mddsagprsp |
| 301 | tgewlprdse chlcmsvttq agnsseqaip qamlqacvgs wldrekckqf veqhtpqllt |
| 361 | lvprgwdaht tcqalgvcgt mssplqcihs pdl |

Surfactant protein C, pulmonary surfactant-associated protein C isoform 1
precursor, NP_001165881.1, NP_003009.2 (SEQ ID NO: 143)

| | |
|---|---|
| 1 | mdvgskevlm esppdysaap rgrfgipccp vhlkrllivv vvvvlivvvi vgallmglhm |
| 61 | sqkhtemvle msigapeaqq rlalsehlvt tatfsigstg lvvydyqqll iaykpapgtc |
| 121 | cyimkiapes ipslealtrk vhnfqmecsl qakpavptsk lgqaegrdag sapsggdpaf |
| 181 | lgmaysticg evplyyi |

Surfactant protein C, pulmonary surfactant-associated protein C isoform 2
precursor, NP_001165828.1, NP_001304707.1, NP_001304709.1 (SEQ ID NO: 144)

| | |
|---|---|
| 1 | mdvgskevlm esppdysaap rgrfgipccp vhlkrllivv vvvvlivvvi vgallmglhm |
| 61 | sqkhtemvle msigapeaqq rlalsehlvt tatfsigstg lvvydyqqll iaykpapgtc |
| 121 | cyimkiapes ipslealtrk vhnfqakpav ptsklgqaeg rdagsapsgg dpaflgmavs |
| 181 | ticgevplyy i |

Surfactant protein C, pulmonary surfactant-associated protein C isoform 3
precursor, NP_001304708.1 (SEQ ID NO: 145)

| | |
|---|---|
| 1 | mdvgskevlm esppvlemsi gapeaqqrla lsehlvttat fsigstglvv ydyqqlliay |
| 61 | kpapgtccyi mkiapesips lealtrkvhn fqmecslqak pavptsklgq aegrdagsap |
| 121 | sggdpaflgm aystlcgevp lyyi |

Surfactant protein D, pulmonary surfactant-associated protein D precursor,
NP_003010.4 (SEQ ID NO: 146)

| | |
|---|---|
| 1 | mllfllsalv lltqplgyle aemktyshrt mpsactlvmc ssvesglpgr dgrdgregpr |
| 61 | gekgdpglpg aagqagmpgq agpvgpkgdn gsvgepgpkg dtgpsgppgp pgvpgpagre |
| 121 | gplgkqgnig pqgkpgpkge agpkgevgap gmqgsagarg lagpkgergv pgergvpgnt |
| 181 | gaagsagamg pqgspgargp pglkgdkgip gdkgakgesg lpdvaslrqq vealqgqvqh |
| 241 | lqaafsqykk velfpngqsv gekifktagf vkpfteaqll ctqaggqlas prsaaenaal |
| 301 | qqlvvaknea aflsmtdskt egkftyptge slvysnwapg epnddggsed cveiftngkw |
| 361 | ndracgekrl vvcef |

Solute carrier family 2 member 5, solute carrier family 2, facilitated
glucose transporter member 5 isoform 1, NP_001315548.1, NP_003030.1 (SEQ ID
NO: 147)

| | |
|---|---|
| 1 | meqqdqsmke grltlvlala tliaafgssf qygynvaavn spallmqqfy netyygrtge |
| 61 | fmedfpltll wsvtvsmfpf ggfigsllvg plvnkfgrkg allfnnifsi vpailmgcsr |
| 121 | vatsfeliii srllvgicag vssnvvpmyl gelapknlrg algvvpqlfi tvgilvaqif |
| 181 | glrnllanvd gwpillgltg vpaalqllll pffpespryl liqkkdeaaa kkalqtlrgw |
| 241 | dsvdrevaei rqedeaekaa gfisvlklfr mrslrwqlls iivlmggqql sgvnaiyyya |
| 301 | dqiylsagvp eehvqyvtag tgavnvvmtf cavfvvellg rrlllllgfs icliaccvlt |
| 361 | aalalqdtvs wmpyisivcv isyvighalg pspipallit eiflqssrps afmvggsvhw |
| 421 | lsnftvglif pfiqeglgpy sfivfavicl lttiyifliv petkaktfie inqiftkmnk |
| 481 | vsevypekee lkelppvtse q |

Solute carrier family 2 member 5, solute carrier family 2, facilitated
glucose transporter member 5 isoform 2, NP_001129057.1 (SEQ ID NO: 148)

| | |
|---|---|
| 1 | meqqdqsmke grltlvlala tliaafgssf qygynvaavn spallmqqfy netyygrtge |
| 61 | fmedfpltll wsvtvsmfpf ggfigsllvg plvnkfgrkg allfnnifsi vpailmgcsr |
| 121 | vatsfeliii srllvgicag vssnvvpmyl gelapknlrg algvvpqlfi tvgilvaqif |
| 181 | glrnllanvd gefrtsrehp hpftttlgpl lvfqshhhrt glsadwsllt gwmslggpsc |
| 241 | pept |

Solute carrier family 2 member 5, solute carrier family 2, facilitated
glucose transporter member 5 isoform 3 NP_001315549.1 (SEQ ID NO: 149)

| | |
|---|---|
| 1 | mgttwllstp qhwtgefmed fpltllwsvt vsmfpfggfi gsllvgplvn kfgrkgallf |
| 61 | nnifsivpai lmgcsrvats feliiisrll vgicagvssn vvpmylgela pknlrgalgv |
| 121 | vpqlfitvgi lvaqifglrn llanvdgwpi llgltgvpaa lqllllpffp esprylliqk |
| 181 | kdeaaakkal qtlrgwdsvd revaeirqed eaekaagfis vlklfrmrsl rwqllsiivl |
| 241 | mggqqlsgvn aiyyyadqiy lsagvpeehv qyvtagtgav nvvmtfcavf vvellgrrll |
| 301 | lllgfsicli accvltaala lqdtvswmpy isivcvisyv ighalgpspi palliteifl |
| 361 | qssrpsafmv ggsvhwlsnf tvglifpfiq eglgpysfiv faviclltti yiflivpetk |
| 421 | aktfieinqi ftkmnkvsev ypekeelkel ppvtseq |

Solute carrier family 2 member 5, solute carrier family 2, facilitated
glucose transporter member 5 isoform 4, NP_001315550.1 (SEQ ID NO: 150)

| | |
|---|---|
| 1 | mylgelapkn lrgalgvvpq lfitvgilva qifglrnlla nvdgwpillg ltgvpaalql |
| 61 | lllpffpesp rylliqkkde aaakkalqtl rgwdsvdrev aeirqedeae kaagfisvlk |
| 121 | lfrmrslrwq llsiivlmgg qqlsgvnaiy yyadqiylsa gvpeehvqyv tagtgavnvv |
| 181 | mtfcavfvve llgrrlllll gfsicliacc vltaalalqd tvswmpyisi vcvisyvigh |
| 241 | algpspipal liteiflqss rpsafmvggs vhwlsnftvg lifpfiqegl gpysfivfav |
| 301 | icllttiyif livpetkakt fieinqiftk mnkvsevype keelkelppv tseq |

Sperm associated antigen 9, C-Jun-amino-terminal kinase-interacting protein 4
isoform 1, NP_001124000.1 (SEQ ID NO: 151)

| | |
|---|---|
| 1 | meledgvvyq eepggsgavm servsglags iyreferlig rydeevvkel mplvvavlen |
| 61 | ldsvfaqdqe hqvelellrd dneqlitqye rekalrkhae ekfiefedsq eqekkdlqtr |
| 121 | veslesqtrq lelkaknyad qisrleerea elkkeynalh qrhtemihny mehlertklh |
| 181 | qlsgsdqles tahsrirker pislgifplp agdglltpda qkggetpgse qwkfqelsqp |
| 241 | rshtslkvsn spepqkaveq edelsdvsqg gskattpast ansdvatipt dtplkeeneg |
| 301 | fvkvtdapnk seiskhievq vaqetrnvst gsaeneekse vqaiiestpe ldmdkdlsgy |
| 361 | kgsstptkgi enkafdrnte slfeelssag sgligdvdeg adllgmgrev enlilentql |
| 421 | letknalniv kndliakvde ltcekdvlqg eleavkqakl kleeknrele eelrkaraea |
| 481 | edarqkakdd ddsdiptaqr krftrvemar vlmernqyke rlmelqeavr wtemirasre |
| 541 | npamqekkrs siwqffsrlf ssssnttkkp eppvnlkyna ptshvtpsvk krsstlsqlp |
| 601 | gdkskafdfl seeteaslas rreqkreqyr qvkahvqked grvqafgwsl pqkykqvtng |
| 661 | qgenkmknlp vpvylrplde kdtsmklwca vgvnlsggkt rdggsvvgas vfykdvagld |
| 721 | tegskqrsas qssldkldqe lkeqqkelkn qeelsslvwi ctsthsatkv liidavqpgn |
| 781 | ildsftvcns hvlciasvpg aretdypage dlsesgqvdk aslcgsmtsn ssaetdsllg |
| 841 | gitvvgcsae gvtgaatsps tngaspvmdk ppemeaense vdenvptaee ateategnag |
| 901 | saedtvdisq tgvytehvft dplgvqiped lspvyqssnd sdaykdqisv lpneqdlvre |
| 961 | eaqkmssllp tmwlgaqngc lyvhssvaqw rkclhsiklk dsilsivhvk givlvaladg |
| 1021 | tlaifhrgvd gqwdlsnyhl ldlgrphhsi rcmtvvhdkv wcgyrnkiyv vqpkamkiek |
| 1081 | sfdahprkes qvrqlawvgd gvwvsirlds tlrlyhahty qhlqdvdiep yvskmlgtgk |
| 1141 | lgfsfvrita lmvscnrlwv gtgngviisi pltetnktsg vpgnrpgsvi rvygdensdk |
| 1201 | vtpgtfipyc smahaqlcfh ghrdavkffv avpgqvispq ssssgtdltg dkagpsaqep |
| 1261 | gsqtplksml visggegyid frmgdegges ellgedlple psvtkaersh livwqvmygn |
| 1321 | e |

Sperm associated antigen 9, C-Jun-amino-terminal kinase-interacting protein 4
isoform 2, NP_001123999.1 (SEQ ID NO: 152)

| | |
|---|---|
| 1 | meledgvvyq eepggsgavm servsglags iyreferlig rydeevvkel mplvvavlen |
| 61 | ldsvfaqdqe hqvelellrd dneqlitqye rekalrkhae ekfiefedsq eqekkdlqtr |
| 121 | veslesqtrq lelkaknyad qisrleerea elkkeynalh qrhtemihny mehlertklh |
| 181 | qlsgsdqles tahsrirker pislgifplp agdglltpda qkggetpgse qwkfqelsqp |
| 241 | rshtslkdel sdvsqggska ttpastansd vatiptdtpl keenegfvkv tdapnkseis |
| 301 | khievqvaqe trnvstgsae neeksevqai iestpeldmd kdlsgykgss tptkgienka |
| 361 | fdrnteslfe elssagsgli gdvdegadll gmgrevenli lentqlletk nalnivkndl |
| 421 | iakvdeltce kdvlqgelea vkqaklklee knreleeelr karaeaedar qkakddddsd |
| 481 | iptaqrkrft rvemarvlme rnqykerlme lqeavrwtem irasrenpam qekkrssiwq |
| 541 | fvptrfsrlf ssssnttkkp eppvnlkyna ptshvtpsvk krsstlsqlp gdkskafdfl |
| 601 | seeteaslas rreqkreqyr qvkahvqked grvqafgwsl pqkykqvtng qgenkmknlp |
| 661 | vpvylrplde kdtsmklwca vgvnlsggkt rdggsvvgas vfykdvagld tegskqrsas |
| 721 | qssldkldqe lkeqqkelkn qeelsslvwi ctsthsatkv liidavqpgn ildsftvcns |
| 781 | hvlciasvpg aretdypage dlsesgqvdk aslcgsmtsn ssaetdsllg gitvvgcsae |
| 841 | gvtgaatsps tngaspvmdk ppemeaense vdenvptaee ateategnag saedtvdisq |
| 901 | tgvytehvft dplgvqiped lspvyqssnd sdaykdqisv lpneqdlvre eaqkmssllp |
| 961 | tmwlgaqngc lyvhssvaqw rkclhsiklk dsilsivhvk givlvaladg tlaifhrgvd |
| 1021 | gqwdlsnyhl ldlgrphhsi rcmtvvhdkv wcgyrnkiyv vqpkamkiek sfdahprkes |
| 1081 | qvrqlawvgd gvwvsirlds tlrlyhahty qhlqdvdiep yvskmlgtgk lgfsfvrita |
| 1141 | lmvscnrlwv gtgngviisi pltetnktsg vpgnrpgsvi rvygdensdk vtpgtfipyc |
| 1201 | smahaqlcfh ghrdavkffv avpgqvispq ssssgtdltg dkagpsaqep gsqtplksml |
| 1261 | visggegyid frmgdegges ellgedlple psvtkaersh livwqvmygn e |

Sperm associated antigen 9, C-Jun-amino-terminal kinase-interacting protein 4
isoform 3, NP_003962.3 (SEQ ID NO: 153)

| | |
|---|---|
| 1 | meledgvvyq eepggsgavm servsglags iyreferlig rydeevvkel mplvvavlen |
| 61 | ldsvfaqdqe hqvelellrd dneqlitqye rekalrkhae ekfiefedsq eqekkdlqtr |
| 121 | veslesqtrq lelkaknyad qisrleerea elkkeynalh qrhtemihny mehlertklh |
| 181 | qlsgsdqles tahsrirker pislgifplp agdglltpda qkggetpgse qwkfqelsqp |
| 241 | rshtslkdel sdvsqggska ttpastansd vatiptdtpl keenegfvkv tdapnkseis |
| 301 | khievqvaqe trnvstgsae neeksevqai iestpeldmd kdlsgykgss tptkgienka |
| 361 | fdrnteslfe elssagsgli gdvdegadll gmgrevenli lentqlletk nalnivkndl |
| 421 | iakvdeltce kdvlqgelea vkqaklklee knreleeelr karaeaedar qkakddddsd |
| 481 | iptaqrkrft rvemarvlme rnqykerlme lqeavrwtem irasrenpam qekkrssiwq |
| 541 | ffsrlfssss nttkkpeppv nlkynaptsh vtpsvkkrss tlsqlpgdks kafdflseet |
| 601 | easlasrreq kreqyrqvka hvqkedgrvq afgwslpqky kqvtngqgen kmknlpvpvy |
| 661 | lrpldekdts mklwcavgvn lsggktrdgg svvgasvfyk dvagldtegs kqrsasqssl |
| 721 | dkldqelkeq qkelknqeel sslvwictst hsatkvliid avqpgnilds ftvcnshvlc |
| 781 | iasvpgaret dypagedlse sgqvdkaslc gsmtsnssae tdsllggitv vgcsaegvtg |
| 841 | aatspstnga spvmdkppem eaensevden vptaeeatea tegnagsaed tvdisqtgvy |
| 901 | tehvftdplg vqipedlspv yqssndsday kqgisvlpne qdlvreeaqk mssllptmwl |
| 961 | gaqngclyvh ssvaqwrkcl hsiklkdsil sivhvkgivl valadgtlai fhrgvdgqwd |
| 1021 | lsnyhlldlg rphhsircmt vvhdkvwcgy rnkiyvvqpk amkieksfda hprkesqvrq |
| 1081 | lawvgdgvwv sirldstlrl yhahtyghlq dvdiepyvsk mlgtgklgfs fvritalmvs |
| 1141 | cnrlwvgtgn gviisiplte tnktsgvpgn rpgsvirvyg densdkvtpg tfipycsmah |
| 1201 | aqlcfhghrd avkffvavpg qvispqssss gtdltgdkag psaqepgsqt plksmlvisg |
| 1261 | gegyidfrmg deggesellg edlplepsvt kaershlivw qvmygne |

Sperm associated antigen 9, C-Jun-amino-terminal kinase-interacting protein 4
isoform 4, NP_001238900.1 (SEQ ID NO: 154)

| | |
|---|---|
| 1 | mspgcmllfv fgfvggavvi nsailvslsv lllvhfsist gvpaltqnlp rilrkerpis |
| 61 | lgifplpagd glltpdaqkg getpgseqwk fqelsqprsh tslkdelsdv sqggskattp |
| 121 | astansdvat iptdtplkee negfvkvtda pnkseiskhi evqvaqetrn vstgsaenee |
| 181 | ksevqaiies tpeldmdkdl sgykgsstpt kgienkafdr nteslfeels sagsgligdv |
| 241 | degadllgmg revenlilen tqlletknal nivkndliak vdeltcekdv lqgeleavkg |
| 301 | aklkleeknr eleeelrkar aeaedarqka kddddsdipt aqrkrftrve marvlmernq |
| 361 | ykerlmelqe avrwtemira srenpamgek krssiwqffs rlfssssntt kkpeppvnlk |
| 421 | ynaptshvtp svkkrsstls qlpgdkskaf dflseeteas lasrreqkre qyrqvkahvq |
| 481 | kedgrvqafg wslpqkykqv tngqgenkmk nlpvpvylrp ldekdtsmkl wcavgvnlsg |
| 541 | gktrdggsvv gasvfykdva gldtegskqr sasqssldkl dqelkeqqke lknqeelssl |
| 601 | vwictsthsa tkvliidavq pgnildsftv cnshvlcias vpgaretdyp agedlsesgq |
| 661 | vdkaslcgsm tsnssaetds llggitvvgc saegvtgaat spstngaspv mdkppemeae |
| 721 | nsevdenvpt aeeateateg nagsaedtvd isqtgvyteh vftdplgvqi pedlspvyqs |
| 781 | sndsdaykdq isvlpneqdl vreeaqkmss llptmwlgaq ngclyvhssv aqwrkclhsi |
| 841 | klkdsilsiv hvkgivlval adgtlaifhr gvdgqwdlsn yhlldlgrph hsircmtvvh |
| 901 | dkvwcgyrnk iyvvqpkamk ieksfdahpr kesqvrqlaw vgdgvwvsir ldstlrlyha |
| 961 | htyqhlqdvd iepyvskmlg tgklgfsfvr italmvscnr lwvgtgngvi isipltetvi |
| 1021 | lhqgrllglr anktsgvpgn rpgsvirvyg densdkvtpg tfipycsmah aqlcfhghrd |
| 1081 | avkffvavpg qvispqssss gtdltgdkag psaqepgsqt plksmlvisg gegyidfrmg |
| 1141 | deggesellg edlplepsvt kaershlivw qvmygne |

SGT1 homolog, MIS12 kinetochore complex assembly cochaperone, protein SGT1
homolog isoform A, NP_006695.1 (SEQ ID NO: 155)

| | |
|---|---|
| 1 | maaaaagtat sqrffqsfsd alidedpqaa leeltkaleq kpddaqyycq raychillgn |
| 61 | ycvavadakk slelnpnnst amlrkgicey heknyaaale tftegqklds adanfsvwik |
| 121 | rcqeaqngse sevwthqski kydwyqtesq vvitlmiknv qkndvnvefs ekelsalvkl |
| 181 | psgedynlkl ellhpiipeq stfkvlstki eiklkkpeav rweklegqgd vptpkqfvad |
| 241 | vknlypsssp ytrnwdklvg eikeeeknek legdaalnrl fqqiysdgsd evkramnksf |
| 301 | mesggtvlst nwsdvgkrkv einppddmew kky |

SGT1 homolog, MIS12 kinetochore complex assembly cochaperone, protein SGT1
homolog isoform B, NP_001124384.1 (SEQ ID NO: 156)

| | |
|---|---|
| 1 | maaaaagtat sqrffqsfsd alidedpqaa leeltkaleq kpddaqyycq raychillgn |
| 61 | ycvavadakk slelnpnnst amlrkgicey heknyaaale tftegqkldi etgfhrvgqa |
| 121 | glqlltssdp paldsqsagi tgadanfsvw ikrcqeaqng sesevwthqs kikydwyqte |
| 181 | sqvvitlmik nvqkndvnve fsekelsalv klpsgedynl klellhpiip eqstfkvlst |
| 241 | kieiklkkpe avrweklegq gdvptpkqfv advknlypss spytrnwdkl vgeikeeekn |
| 301 | eklegdaaln rlfqqiysdg sdevkramnk sfmesggtvl stnwsdvgkr kveinppddm |
| 361 | ewkky |

SGT1 homolog, MIS12 kinetochore complex assembly cochaperone, protein SGT1
homolog isoform C, NP_001307760.1 (SEQ ID NO: 157)

| | |
|---|---|
| 1 | mlsqkevava dakkslelnp nnstamlrkg iceyheknya aaletftegq kldsadanfs |
| 61 | vwikrcqeaq ngsesevwth qskikydwyq tesqvvitlm iknvqkndvn vefsekelsa |
| 121 | lvklpsgedy nlklellhpi ipeqstfkvl stkieiklkk peavrwekle gqgdvptpkq |
| 181 | fvadvknlyp ssspytrnwd klvgeikeee kneklegdaa lnrlfgqiys dgsdevkram |
| 241 | nksfmesggt vlstnwsdvg krkveinppd dmewkky |

Sulfotransferase family 1C member 2, sulfotransferase 1C2 isoform a,
NP_001047.1 (SEQ ID NO: 158)

| | |
|---|---|
| 1 | maltsdlgkq iklkevegtl lqpatvdnws qiqsfeakpd dllictypka gttwiqeivd |
| 61 | mieqngdvek cqraiiqhrh pfiewarppq psgvekakam psprilkthl stqllppsfw |
| 121 | ennckflyva rnakdcmvsy yhfqrmnhml pdpgtweeyf etfingkvvw gswfdhvkgw |
| 181 | wemkdrhqil flfyedikrd pkheirkvmq fmgkkvdetv ldkivqetsf ekmkenpmtn |
| 241 | rstvsksild qsissfmrkg tvgdwknhft vaqnerfdei yrrkmegtsi nfcmel |

Sulfotransferase family 1C member 2, sulfotransferase 1C2 isoform b,
NP_789795.1 (SEQ ID NO: 159)

| | |
|---|---|
| 1 | maltsdlgkq iklkevegtl lqpatvdnws qiqsfeakpd dllictypka gttwiqeivd |
| 61 | mieqngdvek cqraiiqhrh pfiewarppq psetgfhhva qaglkllsss nppastsqsa |
| 121 | kitdllppsf wennckflyv arnakdcmvs yyhfqrmnhm lpdpgtweey fetfingkvv |
| 181 | wgswfdhvkg wwemkdrhqi lflfyedikr dpkheirkvm qfmgkkvdet vldkivqets |
| 241 | fekmkenpmt nrstvsksil dqsissfmrk gtvgdwknhf tvaqnerfde iyrrkmegts |
| 301 | infcmel |

Transmembrane protein 52B, isoform 1, NP_694567.1 (SEQ ID NO: 160)

| | |
|---|---|
| 1 | mswrpqpcci sscclttdwv hlwyiwllvv igallllcgl tslcfrcccl srqqngedgg |
| 61 | pppcevtvia fdhdstlqst itslqsvfgp aarrilavah shsslgqlps sldtlpgyee |
| 121 | alhmsrftva mcgqkapdlp pvpeekqlpp tekestrivd swn |

Transmembrane protein 52B, isoform 2 precursor, NP_001073283.1 (SEQ ID NO:
161)

| | |
|---|---|
| 1 | mgvrvhvvaa sallyfills gtrceencgn pehclttdwv hlwyiwllvv igallllcgl |
| 61 | tslcfrcccl srqqngedgg pppcevtvia fdhdstlqst itslqsvfgp aarrilavah |
| 121 | shsslgqlps sldtlpgyee alhmsrftva mcgqkapdlp pvpeekqlpp tekestrivd |
| 181 | swn |

Exportin 7, NP_055839.3 (SEQ ID NO: 162)

| | |
|---|---|
| 1 | madhvqslaq lenlckqlye ttdtttrlqa ekalveftns pdclskcqll lergsssysq |
| 61 | llaatcltkl vsrtnnplpl eqridirnyv lnylatrpkl atfvtqaliq lyaritklgw |
| 121 | fdcqkddyvf rnaitdvtrf lqdsveycii gvtilsqltn einqadtthp ltkhrkiass |
| 181 | frdsslfdif tlscnllkqa sgknlnlnde sqhgllmqll klthnclnfd figtstdess |
| 241 | ddlctvqipt swrsafldss tlqlffdlyh sippsfsplv lsclvqiasv rrslfnnaer |
| 301 | akflshlvdg vkrilenpqs lsdpnnyhef crllarlksn yqlgelvkve nypevirlia |
| 361 | nftvtslqhw efapnsvhyl lslwqrlaas vpyvkateph mletytpevt kayitsrles |
| 421 | vhiilrdgle dpledtglvq qqldqlstig rceyektcal lvqlfdqsaq syqellqsas |
| 481 | aspmdiavqe grltwlvyii gaviggrvsf astdeqdamd gelvcrvlql mnitdsrlaq |
| 541 | agneklelam lsffeqfrki yigdqvqkss klyrrlsevl glndetmvls vfigkiitnl |
| 601 | kywgrcepit sktlqllndl sigyssvrkl vklsavqfml nnhtsehfsf lginnqsnlt |
| 661 | dmrcrttfyt algrllmvdl gededqyeqf mlpltaafea vaqmfstnsf neqeakrtlv |
| 721 | glvrdlrgia fafnaktsfm mlfewiypsy mpilqraiel wyhdpacttp vlklmaelvh |
| 781 | nrsqrlqfdv sspngillfr etskmitmyg nriltlgevp kdqvyalklk gisicfsmlk |
| 841 | aalsgsyvnf gvfrlygdda ldnalqtfik lllsiphsdl ldypklsqsy ysllevltqd |
| 901 | hmnfiaslep hvimyilssi segltaldtm vctgccscld hivtylfkql srstkkrttp |
| 961 | lnqesdrflh imqqhpemiq qmlstvinii ifedcrnqws msrpllglil lnekyfsdlr |
| 1021 | nsivnsqppe kqqamhlcfe nlmegiernl ltknrdrftq nlsafrrevn dsmknstygv |
| 1081 | nsndmms |

YES proto-oncogene 1, Src family tyrosine kinase, tyrosine-protein kinase
Yes, NP_005424.1 (SEQ ID NO: 163)

| | |
|---|---|
| 1 | mgcikskenk spaikyrpen tpepvstsvs hygaepttvs pcpsssakgt avnfsslsmt |
| 61 | pfggssgvtp fggasssfsv vpssypaglt ggvtifvaly dyearttedl sfkkgerfqi |
| 121 | inntegdwwe arsiatgkng yipsnyvapa dsiqaeewyf gkmgrkdaer lllnpgngrg |
| 181 | iflvresett kgayslsird wdeirgdnvk hykirkldng gyyittraqf dtlqklvkhy |
| 241 | tehadglchk lttvcptvkp qtqqlakdaw eipreslrle vklgqgcfge vwmgtwngtt |
| 301 | kvaiktlkpg tmmpeaflqe aqimkklrhd klvplyavvs eepiyivtef mskgslldfl |
| 361 | kegdgkylkl pglvdmaaqi adgmayierm nyihrdlraa nilvgenlvc kiadfglarl |
| 421 | iedneytarq gakfpikwta peaalygrft iksdvwsfgi lqtelvtkgr vpypgmvnre |
| 481 | vleqvergyr mpcpqgcpes lhelmnlcwk kdpderptfe yiqsfledyf tatepqyqpg |
| 541 | enl |

Coiled-coil domain containing 80, coiled-coil domain-containing 80 precursor,
NP_955805.1, NP_955806.1 (SEQ ID NO: 164)

| | |
|---|---|
| 1 | mtwrmgprft mllamwlvcg sephphatir gshggrkvpl vspdssrpar flrhtgrsrg |
| 61 | ierstleepn lqplqrrrsv pvlrlarpte pparsdinga avrpeqrpaa rgspremird |
| 121 | egssarsrml rfpsgssspn ilasfagknr vwvisaphas egyyrlmmsl lkddvycela |
| 181 | erhiqqivlf hqageeggkv rritsegqil eqpldpslip klmsflklek gkfgmvllkk |
| 241 | tlqveerypy pvrleamyev idqgpirrie kirqkgfvqk ckasgvegqv vaegndgggg |
| 301 | agrpslgsek kkedprraqv pptresrvkv lrklaatapa lpqppstpra ttlppapatt |
| 361 | vtrstsravt vaarpmttta fpttqrpwtp spshrppttt evitarrpsv senlyppsrk |
| 421 | dqhrerpqtt rrpskatsle sftnapptti sepstraagp grfrdnrmdr rehghrdpnv |
| 481 | vpgppkpake kppkkkaqdk ilsneyeeky dlsrptasql edelqvgnvp lkkakeskkh |
| 541 | eklekpekek kkkmknenad kllksekqmk ksekkskqek ekskkkkggk teqdgyqkpt |
| 601 | nkhftqspkk svadllgsfe gkrrlllita pkaennmyvq qrdeylesfc kmatrkisvi |
| 661 | tifgpvnnst mkidhfqldn ekpmrvvdde dlvdqrlise lrkeygmtyn dffmvltdvd |
| 721 | lrvkqyyevp itmksvfdli dtfqsrikdm ekqkkegivc kedkkqslen flsrfrwrrr |
| 781 | llvisapnde dwaysqqlsa lsgqacnfgl rhitilkllg vgeevggvle lfpingssvv |
| 841 | eredvpahlv kdirnyfqvs peyfsmllvg kdgnvkswyp spmwsmvivy dlidsmqlrr |
| 901 | qemaiqqslg mrcpedeyag ygyhsyhqgy qdgyqddyrh hesyhhgypy |

Acrosin-binding protein precursor NP_115878.2 (SEQ ID NO: 165)

| | |
|---|---|
| 1 | mrkpaagflp sllkvlllpl apaaaqdstq astpgsplsp teyerffall tptwkaettc |
| 61 | rlrathgcrn ptivqldqye nhglvpdgav csnlpyaswf esfcqfthyr csnhvyyakr |
| 121 | vlcsqpvsil spntlkeiea saevspttmt spisphftvt erqtfqpwpe rlsnnveell |
| 181 | qsslslggqe qapehkqeqg vehrqeptqe hkqeegqkqe eqeeeqeeeg kqeegqgtke |
| 241 | greaysqlqt dsepkfhses lssnpssfap rvrevestpm imeniqelir saqeidemne |
| 301 | iydensywrn qnpgsllqlp hteallvlcy siventciit ptakawkyme eeilgfgksv |
| 361 | cdslgrrhms tcalcdfcsl kleqchseas lqrqqcdtsh ktpfvsplla sqslsignqv |
| 421 | gspesgrfyg ldlygglhmd fwcarlatkg cedvrvsgwl qteflsfqdg dfptkicdtd |
| 481 | yiqypnycsf ksqqclmrnr nrkvsrmrcl qnetysalsp gksedvvlrw sqefstltlg |
| 541 | qfg |

Alpha-fetoprotein, isoform 1 NP_001125.1 (SEQ ID NO: 166)

| | |
|---|---|
| 1 | mkwvesifli fllnftesrt lhrneygias ildsyqctae isladlatif faqfvqeaty |
| 61 | kevskmvkda ltaiekptgd eqssgclenq lpafleelch ekeilekygh sdccsqseeg |
| 121 | rhncflahkk ptpasiplfq vpepvtscea yeedretfmn kfiyeiarrh pflyaptill |
| 181 | waarydkiip scckaenave cfqtkaatvt kelresslln qhacavmknf gtrtfqaitv |
| 241 | tklsqkftkv nfteiqklvl dvahvhehcc rgdvldclqd gekimsyics qqdtlsnkit |
| 301 | eccklttler gqciihaend ekpeglspnl nrflgdrdfn qfssgeknif lasfvheysr |
| 361 | rhpqlaysvi lrvakgyqel lekcfqtenp lecqdkgeee lqkyiqesqa lakrscglfq |
| 421 | klgeyylqna flvaytkkap qltsselmai trkmaataat ccqlsedkll acgegaadii |
| 481 | ighlcirhem tpvnpgvgqc ctssyanrrp cfsslvvdet yvppafsddk fifhkdlcqa |
| 541 | qgvalqtmkq eflinlvkqk pqiteeqlea viadfsglle kccqgqeqev cfaeegqkli |
| 601 | sktraalgv |

Alpha-fetoprotein, isoform 2 NP_001341646.1 (SEQ ID NO: 167)

| | |
|---|---|
| 1 | mnkfiyeiar rhpflyapti llwaarydki ipscckaena vecfqtkaat vtkelressl |
| 61 | lnqhacavmk nfgtrtfqai tvtklsqkft kvnfteiqkl vldvahvheh ccrgdvldcl |
| 121 | qdgerimsyi csqqdtlsnk iteccklttl ergqciihae ndekpeglsp nlnrflgdrd |
| 181 | fnqfssgekn iflasfvhey srrhpqlavs vilrvakgyq ellekcfqte nplecqdkge |
| 241 | eelqkyiqes qalakrscgl fqklgeyylq naflvaytkk apqltsselm aitrkmaata |
| 301 | atccqlsedk llacgegaad iiighlcirh emtpvnpgvg qcctssyanr rpcfsslvvd |
| 361 | etyvppafsd dkfifhkdlc qaqgvalqtm kqeflinlvk qkpqiteeql eaviadfsgl |
| 421 | lekccqgqeq evcfaeegqk lisktraalg v |

Absent in melanoma 1 protein NP_001615.2 (SEQ ID NO: 168)

| | |
|---|---|
| 1 | mplsppaqgd pgepspcrpp kkhttfhlwr skkkqqpapp dcgvfvphpl papagearal |
| 61 | dvvdgkyvvr dsqefplhcg esqffhttse algslllesg ifkksraqpp ednrrkpvlg |
| 121 | klgtlftagr rrnsrngles ptrsnakpls pkdvvaspkl peresersrs qssqlkqtdt |
| 181 | seegsprenp reaegelpes ggpaappdae lsprwsssaa avavqqchen dspqleplea |
| 241 | egepfpdatt takqlhsspg nssrqenaet parspgedas pgagheqeaf lgvrgapgsp |
| 301 | tqerpagglg eapngapsvc aeegslgprn arsqppkgas dlpgeppaeg aahtassaqa |
| 361 | dctarpkgha hpakvltldi ylsktegaqv depvvitpra edcgdwddme krssgrrsgr |
| 421 | rrgsqkstds pgadaelpes aarddavfdd evapnaasdn asaekkvksp raaldggvas |
| 481 | aaspeskpsp gtkgqlrges drskqpppas sptkrkgrsr aleavpappa sgprapakes |
| 541 | ppkrvpdpsp vtkgtaaesg eeaaraipre lpvksssllp eikpehkrgp lpnhfngrae |
| 601 | ggrsrelgra agapgasdad glkprnhfgv grstvttkvt lpakpkhvel nlktpknlds |
| 661 | lgnehnpfsq pvhkgntatk islfenkrtn ssprhtdirg qrntpasskt fvgraklnla |
| 721 | kkakemeqpe kkvmpnspqn gvlvketaie tkvtvseeei lpatrgmngd ssenqalgpq |
| 781 | pnqddkadvq tdagclsepv asalipvkdh kllekedsea adskslvlen vtdtaqdipt |
| 841 | tvdtkdlppt ampkpqhtfs dsqspaessp gps1s1sapa pgdvpkdtcv qspissfpct |
| 901 | dlkvsenhkg cvlpvsrqnn ekmpllelgg ettpplster speavgsecp srvlvqvrsf |
| 961 | vlpvestqdv ssqvipesse vrevqlptch snepevvsva scappqeevl gnehshctae |
| 1021 | laaksgpqvi ppasektlpi qaqsqgsrtp lmaessptns pssgnhlatp qrpdqtvtng |
| 1081 | qdspasllni sagsddsvfd sssdmekfte iikqmdsavc mpmkrkkarm pnspaphfam |
| 1141 | ppihedhlek vfdpkvftfg lgkkkesqpe mspalhlmqn ldtksklrpk rasaeqsvlf |
| 1201 | kslhtntngn seplvmpein dkenrdvtng gikrsrleks alfssllssl pqdkifspsv |
| 1261 | tsvntmttaf stsqngslsq ssysqptteg appcglnkeq snllpdnslk vfnfnsssts |
| 1321 | hsslkspshm ekypqkektk edldsrsnlh lpetkfsels klknddmeka nhiesviksn |
| 1381 | lpncansdtd fmglfkssry dpsisfsgms lsdtmtlrgs vqnklnprpg kvviysepdv |
| 1441 | sekcievfsd iqdcsswsls pvilikvvrg cwilyeqpnf eghsipleeg elelsglwgi |
| 1501 | edilerheea esdkpvvigs irhvvqdyry shidlftepe glgilssyfd dteemqgfgv |
| 1561 | mqktcsmkvh wgtwliyeep gfqgvpfile pgeypdlsfw dteeayigsm rplkmggrkv |
| 1621 | efptdpkvvv yekpffegkc veletgmcsf vmeggeteea tgddhlpfts vgsmkvlrgi |
| 1681 | wvayekpgft ghqylleege yrdwkawggy ngelqslrpi lgdfsnahmi myseknfgsk |
| 1741 | gssidvlgiv anlketgygv ktqsinvlsg vwvayenpdf tgeqyildkg fytsfedwgg |
| 1801 | knckissvqp icldsftgpr rrnqihlfse pqfqghsqsf eettsqidds fstkscrvsg |
| 1861 | gswvvydgen ftgnqyvlee ghypclsamg cppgatfksl rfidvefsep tiilferedf |
| 1921 | kgkkielnae tvnlrslgfn tqirsvqvig giwvtyeygs yrgrqfllsp aevpnwyefs |
| 1981 | gcrqigslrp fvqkriyfrl rnkatglfms tngnledlkl lriqvmedvg addqiwiyqe |
| 2041 | gcikcriaed ccltivgslv tsgsklglal dqnadsqfws lksdgriysk lkpnlvldik |
| 2101 | ggtqydqnhi ilntvskekf tqvweamvly t |

A-kinase anchoring protein 4, isoform 1 NP_003877.2 (SEQ ID NO: 169)

| | |
|---|---|
| 1 | mmaysdttmm sddidwlrsh rgvckvdlyn pegqqdqdrk vicfvdvstl nvedkdykda |
| 61 | assssegnln lgsleekeii vikdtekkdq sktegsvclf kqapsdpvsv lnwllsdlqk |
| 121 | yalgfqhals pststckhkv gdtegeyhra ssencysvya dqvnidylmn rpqnlrlemt |
| 181 | aakntnnnqs psappakpps tqravispdg ecsiddlsfy vnrlsslviq mahkeikekl |
| 241 | egkskclhhs icpspgnker isprtpaski asemayeave ltaaemrgtg eesreggqks |
| 301 | flyselsnks ksgdkqmsqr eskefadsis kglmvyanqv asdmmvslmk tlkvhssgkp |
| 361 | ipasvvlkrv llrhtkeivs dlidscmknl hnitgvlmtd sdfvsavkrn lfnqwkqnat |
| 421 | dimeamlkrl vsaligeeke tksqslsyas lkagshdpkc rnqslefstm kaemkerdkg |
| 481 | kmksdpcksl tsaekvgehi lkegltiwnq kqgnsckvat kacsnkdekg ekinastdsl |
| 541 | akdlivsalk liqyhltqqt kgkdtceedc pgstmgymaq stqyekcggg qsakalsvkq |
| 601 | leshrapgps tcqkenqhld sqkmdmsniv lmliqkllne npfkcedpce genkcsepra |
| 661 | skaasmsnrs dkaeeqcqeh qeldctsgmk qangqfidkl vesvmklcli makysndgaa |
| 721 | laeleeqaas ankpnfrgtr cihsgampqn yqdslghevi vnnqcstnsl qkqlqavlqw |
| 781 | iaasqfnvpm lyfmgdkdgq leklpqvsak aaekgysvgg llqevmkfak erqpdeavgk |
| 841 | varkqlldwl lanl |

A-kinase anchoring protein 4, isoform 2 NP_647450.1 (SEQ ID NO: 170)

| | |
|---|---|
| 1 | msddidwlrs hrgvckvdly npegqqdqdr kvicfvdvst lnvedkdykd aassssegnl |
| 61 | nlgsleekei ivikdtekkd qsktegsvcl fkqapsdpvs vinwllsdlq kyalgfqhal |
| 121 | spststckhk vgdtegeyhr assencysvy adqvnidylm nrpqnlrlem taakntnnnq |
| 181 | spsappakpp stqravispd gecsiddlsf yvnrlsslvi qmahkeikek legkskclhh |
| 241 | sicpspgnke risprtpask iasemayeav eltaaemrgt geesreggqk sflyselsnk |
| 301 | sksgdkqmsq reskefadsi skglmvyanq vasdmmvslm ktlkvhssgk pipasvvlkr |
| 361 | vllrhtkeiv sdlidscmkn lhnitgvlmt dsdfvsavkr nlfnqwkqna tdimeamlkr |
| 421 | lvsaligeek etksqslsya slkagshdpk crnqslefst mkaemkerdk gkmksdpcks |
| 481 | ltsaekvgeh ilkegltiwn qkqgnsckva tkacsnkdek gekinastds lakdlivsal |
| 541 | kliqyhltqq tkgkdtceed cpgstmgyma qstqyekcgg gqsakalsvk qleshrapgp |
| 601 | stcqkenqhl dsqkmdmsni vlmliqklln enpfkcedpc egenkcsepr askaasmsnr |
| 661 | sdkaeeqcqe hqeldctsgm kqangqfidk lvesvmklcl imakysndga alaeleeqaa |
| 721 | sankpnfrgt rcihsgampq nyqdslghev ivnnqcstns lqkqlqavlq wiaasqfnvp |
| 781 | mlyfmgdkdg qleklpqvsa kaaekgysvg gllqevmkfa kerqpdeavg kvarkqlldw |
| 841 | llanl |

ALK tryrosine kinase receptor, isoform 1 NP_004295.2 (SEQ ID NO: 171)

| | |
|---|---|
| 1 | mgaigllwll plllstaavg sgmgtgqrag spaagpplqp replsysrlq rkslavdfvv |
| 61 | pslfrvyard lllppsssel kagrpeargs laldcapllr llgpapgvsw tagspapaea |
| 121 | rtlsrvlkgg svrklrrakq lvlelgeeai legcvgppge aavgllqfnl selfswwirq |
| 181 | gegrlrirlm pekkasevgr egrlsaaira sqprllfqif gtghsslesp tnmpspspdy |
| 241 | ftwnitwimk dsfpflshrs ryglecsfdf pceleysppl hdlrnqswsw rripseeasq |
| 301 | mdlldgpgae rskemprgsf lllntsadsk htilspwmrs ssehctlavs vhrhlqpsgr |
| 361 | yiaqllphne aareillmpt pgkhgwtvlq grigrpdnpf rvaleyissg nrslsavdff |
| 421 | alkncsegts pgskmalqss ftcwngtvlq lgqacdfhqd caqgedesqm crklpvgfyc |
| 481 | nfedgfcgwt qgtlsphtpq wqvrtlkdar fqdhqdhall lsttdvpase satvtsatfp |
| 541 | apiksspcel rmswlirgvl rgnvslvlve nktgkeqgrm vwhvaayegl slwqwmvlpl |
| 601 | ldvsdrfwlq mvawwgqgsr aivafdnisi sldcyltisg edkilqntap ksrnlfernp |
| 661 | nkelkpgens prqtpifdpt vhwlfttcga sgphgptqaq cnnayqnsnl svevgsegpl |
| 721 | kgiqiwkvpa tdtysisgyg aaggkggknt mmrshgvsvl gifnlekddm lyilvgqqge |
| 781 | dacpstnqli qkvcigennv ieeeirvnrs vhewaggggg gggatyvfkm kdgvpvplii |
| 841 | aaggggrayg aktdtfhper lennssvlgl ngnsgaaggg ggwndntsll wagkslqega |
| 901 | tgghscpqam kkwgwetrgg fggggggcss ggggggyigg naasnndpem dgedgvsfis |
| 961 | plgilytpal kvmeghgevn ikhylncshc evdechmdpe shkvicfcdh gtvlaedgvs |
| 1021 | civsptpeph lplslilsvv tsalvaalvl afsgimivyr rkhqelqamq melqspeykl |
| 1081 | sklrtstimt dynpnycfag ktssisdlke vprknitlir glghgafgev yegqvsgmpn |
| 1141 | dpsplqvavk tlpevcseqd eldflmeali iskfnhqniv rcigvslqsl prfillelma |
| 1201 | ggdlksflre trprpsqpss lamldllhva rdiacgcqyl eenhfihrdi aarnclltcp |
| 1261 | gpgrvakigd fgmardiyra syyrkggcam lpvkwmppea fmegiftskt dtwsfgvllw |
| 1321 | eifslgympy psksnqevle fvtsggrmdp pkncpgpvyr imtqcwqhqp edrpnfaiil |
| 1381 | erieyctqdp dvintalpie ygplveeeek vpvrpkdpeg vppllvsqqa kreeerspaa |
| 1441 | ppplpttssg kaakkptaae isvrvprgpa vegghvnmaf sqsnppselh kvhgsrnkpt |
| 1501 | slwnptygsw ftekptkknn piakkephdr gnlglegsct vppnvatgrl pgasllleps |
| 1561 | sltanmkevp lfrlrhfpcg nvnygyqqqg lpleaatapg aghyedtilk sknsmnqpgp |

ALK tyrosin kinese receptor, isoform 2 NP_001340694.1 (SEQ ID NO: 172)

| | |
|---|---|
| 1 | mqmelqspey klsklrtsti mtdynpnycf agktssisdl kevprknitl irglghgafg |
| 61 | evyegqvsgm pndpsplqva vktlpevcse qdeldflmea liiskfnhqn ivrcigvslq |
| 121 | slprfillel maggdlksfl retrprpsqp sslamldllh vardiacgcq yleenhfihr |
| 181 | diaarncllt cpgpgrvaki gdfgmardiy rasyyrkggc amlpvkwmpp eafmegifts |
| 241 | ktdtwsfgvl lweifslgym pypsksnqev lefvtsggrm dppkncpgpv yrimtqcwqh |
| 301 | qpedrpnfai ilerieyctq dpdvintalp ieygplveee ekvpvrpkdp egvppllvsq |
| 361 | qakreeersp aappplptts sgkaakkpta aeisvrvprg pavegghvnm afsqsnppse |
| 421 | lhkvhgsrnk ptslwnptyg swftekptkk nnpiakkeph drgnlglegs ctvppnvatg |
| 481 | rlpgasllle pssltanmke vplfrlrhfp cgnvnygyqq qglpleaata pgaghyedti |
| 541 | lksknsmnqp gp |

Angiopoietin-2, isoform a NP_001138.1 (SEQ ID NO: 173)

| | |
|---|---|
| 1 | mwqivfftls cdlvlaaayn nfrksmdsig kkqyqvqhgs csytfllpem dncrsssspy |
| 61 | vsnavqrdap leyddsvqrl qvlenimenn tqwlmkleny iqdnmkkemv eiqqnavqnq |
| 121 | tavmieigtn llnqtaeqtr kltdveaqvl nqttrlelql lehslstnkl ekqildqtse |
| 181 | inklqdknsf lekkvlamed khiiqlqsik eekdqlqvlv skqnsiieel ekkivtatvn |
| 241 | nsvlqkqqhd lmetvnnllt mmstsnsakd ptvakeeqis frdcaevfks ghttngiytl |
| 301 | tfpnsteeik aycdmeaggg gwtiiqrred gsvdfqrtwk eykvgfgnps geywlgnefv |
| 361 | sqltnqqryv lkihlkdweg neayslyehf ylsseelnyr ihlkgltgta gkissisqpg |
| 421 | ndfstkdgdn dkcickcsqm ltggwwfdac gpsnlngmyy pqrqntnkfn gikwyywkgs |
| 481 | gyslkattmm irpadf |

Angiopoietin-2, isoform b NP_001112359.1 (SEQ ID NO: 174)

| | |
|---|---|
| 1 | mwqivfftls cdlvlaaayn nfrksmdsig kkqyqvqhgs csytfllpem dncrsssspy |
| 61 | vsnavqrdap leyddsvqrl qvlenimenn tqwlmkleny iqdnmkkemv eiqqnavqnq |
| 121 | tavmieigtn llnqtaeqtr kltdveaqvl nqttrlelql lehslstnkl ekqildqtse |
| 181 | inklqdknsf lekkvlamed khiiqlqsik eekdqlqvlv skqnsiieel ekkivtatvn |
| 241 | nsvlqkqqhd lmetvnnllt mmstsnskdp tvakeeqisf rdcaevfksg httngiytlt |
| 301 | fpnsteeika ycdmeagggg wtiiqrredg svdfqrtwke ykvgfgnpsg eywlgnefvs |
| 361 | qltnqqryvl kihlkdwegn eayslyehfy lsseelnyri hlkgltgtag kissisqpgn |
| 421 | dfstkdgdnd kcickcsqml tggwwfdacg psnlngmyyp qrqntnkfng ikwyywkgsg |
| 481 | yslkattmmi rpadf |

Angiopoietin-2, isoform c NP_001112360.1 (SEQ ID NO: 175)

| | |
|---|---|
| 1 | mwqivfftls cdlvlaaayn nfrksmdsig kkqyqvqhgs csytfllpem dncrsssspy |
| 61 | vsnavqrdap leyddsvqrl qvlenimenn tqwlmkvlnq ttrlelqlle hslstnklek |
| 121 | qildqtsein klqdknsfle kkvlamedkh iiqlqsikee kdqlqvlvsk qnsiieelek |
| 181 | kivtatvnns vlqkqqhdlm etvnnlltmm stsnsakdpt vakeeqisfr dcaevfksgh |
| 241 | ttngiytltf pnsteeikay cdmeaggggw tiiqrredgs vdfqrtwkey kvgfgnpsge |
| 301 | ywlgnefvsq ltnqqryvlk ihlkdwegne ayslyehfyl sseelnyrih lkgltgtagk |
| 361 | issisqpgnd fstkdgdndk cickcsqmlt ggwwfdacgp snlngmyypq rqntnkfngi |
| 421 | kwyywkgsgy slkattmmir padf |

Angiopoietin-1, isoform 1 precursor NP_001137.2 (SEQ ID NO: 176)

| | |
|---|---|
| 1 | mtvflsfafl aailthigcs nqrrspensg rrynriqhgq caytfilpeh dgncresttd |
| 61 | qyntnalqrd aphvepdfss qklqhlehvm enytqwlqkl enyivenmks emaqiqqnav |
| 121 | qnhtatmlei gtsllsqtae qtrkltdvet qvlnqtsrle iqllenslst yklekqllqq |
| 181 | tneilkihek nsllehkile megkhkeeld tlkeekenlq glvtrqtyii qelekqlnra |
| 241 | ttnnsvlqkq qlelmdtvhn lvnlctkegv llkggkreee kpfrdcadvy qagfnksgiy |
| 301 | tiyinnmpep kkvfcnmdvn gggwtviqhr edgsldfqrg wkeykmgfgn psgeywlgne |
| 361 | fifaitsqrq ymlrielmdw egnraysqyd rfhignekqn yrlylkghtg tagkqsslil |
| 421 | hgadfstkda dndncmckca lmltggwwfd acgpsnlngm fytagqnhgk lngikwhyfk |
| 481 | gpsyslrstt mmirpldf |

Angiopoietin-1, isoform 2 precursor NP_001186788.1 (SEQ ID NO: 177)

| | |
|---|---|
| 1 | mtvflsfafl aailthigcs nqrrspensg rrynriqhgq caytfilpeh dgncresttd |
| 61 | qyntnalqrd aphvepdfss qklqhlehvm enytqwlqkl enyivenmks emaqiqqnav |
| 121 | qnhtatmlei gtsllsqtae qtrkltdvet qvlnqtsrle iqllenslst yklekqllqq |
| 181 | tneilkihek nsllehkile megkhkeeld tlkeekenlq glvtrqtyii qelekqlnra |
| 241 | ttnnsvlqkq qlelmdtvhn lvnlctkevl lkggkreeek pfrdcadvyq agfnksgiyt |
| 301 | iyinnmpepk kvfcnmdvng ggwtviqhre dgsldfqrgw keykmgfgnp sgeywlgnef |
| 361 | ifaitsqrqy mlrielmdwe gnraysqydr fhignekqny rlylkghtgt agkqsslilh |
| 421 | gadfstkdad ndncmckcal mltggwwfda cgpsnlngmf ytagqnhgkl ngikwhyfkg |
| 481 | psyslrsttm mirpldf |

Angiopoietin-1, isoform 3 precursor NP_001300980.1 (SEQ ID NO: 178)

| | |
|---|---|
| 1 | megkhkeeld tlkeekenlq glvtrqtyii qelekqlnra ttnnsvlqkq qlelmdtvhn |
| 61 | lvnlctkegv llkggkreee kpfrdcadvy qagfnksgiy tiyinnmpep kkvfcnmdvn |
| 121 | gggwtviqhr edgsldfqrg wkeykmgfgn psgeywlgne fifaitsqrq ymlrielmdw |
| 181 | egnraysqyd rfhignekqn yrlylkghtg tagkqsslil hgadfstkda dndncmckca |
| 241 | lmltggwwfd acgpsnlngm fytagqnhgk lngikwhyfk gpsyslrstt mmirpldf |

Ankyrin repeat domain-containing protein 30A NP_443723.2 (SEQ ID NO: 179)

| | |
|---|---|
| 1 | mtkrkktinl niqdaqkrta lhwacvnghe evvtflvdrk cqldvldgeh rtplmkalqc |
| 61 | hqeacanili dsgadinlvd vygntalhya vyseilsvva kllshgavie vhnkasltpl |
| 121 | llsitkrseq ivefllikna nanavnkykc talmlavchg sseivgmllq qnvdvfaadi |
| 181 | cgvtaehyav tcgfhhiheq imeyirklsk nhqntnpegt sagtpdeaap laertpdtae |
| 241 | slvektpdea aplvertpdt aeslvektpd eaaslvegts dkiqclekat sgkfeqsaee |
| 301 | tpreitspak etsekftwpa kgrprkiawe kkedtpreim spaketsekf twaakgrprk |
| 361 | iawekketpv ktgcvarvts nktkvlekgr skmiacptke sstkasandq rfpseskqee |
| 421 | deeyscdsrs lfessakiqv cipesiyqkv meinreveep pkkpsafkpa iemqnsvpnk |
| 481 | afelkneqtl radpmfppes kqkdyeensw dseslcetvs qkdvclpkat hqkeidking |
| 541 | kleespnkdg llkatcgmkv siptkalelk dmqtfkaepp gkpsafepat emqksvpnka |
| 601 | lelkneqtlr adeilpsesk qkdyeenswd teslcetvsq kdvclpkaah qkeidkingk |
| 661 | legspvkdgl lkancgmkvs iptkalelmd mqtfkaeppe kpsafepaie mqksvpnkal |
| 721 | elkneqtlra deilpseskq kdyeesswds eslcetvsqk dvclpkathq keidkingkl |
| 781 | eespdndgfl kapermkvsi ptkalelmdm qtfkaeppek psafepaiem qksvpnkale |
| 841 | lkneqtlrad qmfpseskqk kveenswdse slretvsqkd vcvpkathqk emdkisgkle |
| 901 | dstslskild tvhscerare lqkdhceqrt gkmeqmkkkf cvlkkklsea keiksqlenq |
| 961 | kvkweqelcs vrltlnqeee krrnadilne kireelgrie eqhrkelevk qqleqalriq |
| 1021 | dielksvesn lnqvshthen enyllhencm lkkeiamlkl eiatlkhqyq ekenkyfedi |
| 1081 | kilkeknael qmtlklkees ltkrasqysg qlkvliaent mltsklkekq dkeileaeie |
| 1141 | shhprlasav qdhdqivtsr ksqepafhia gdaclqrkmn vdvsstiynn evlhqplsea |
| 1201 | qrkskslkin lnyagdalre ntivsehaqr dgretqcqmk eaehmyqneq dnvnkhteqq |
| 1261 | esldqklfql qsknmwlqqq lvhahkkadn kskitidihf lerkmqhhll kekneeifny |
| 1321 | nnhlknriyq yekekaeten s |

Androgen receptor, isoform 1 NP_000035.2 (SEQ ID NO: 180)

| | |
|---|---|
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe avsegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgar klkklgnlkl qeegeasstt spteettqkl |
| 661 | tvshiegyec qpiflnvlea iepgvvcagh dnnqpdsfaa llsslnelge rqlvhvvkwa |
| 721 | kalpgfrnlh vddqmaviqy swmglmvfam gwrsftnvns rmlyfapdlv fneyrmhksr |
| 781 | mysqcvrmrh lsqefgwlqi tpqeflcmka lllfsiipvd glknqkffde lrmnyikeld |
| 841 | riiackrknp tscsrrfyql tklldsvqpi arelhqftfd llikshmvsv dfpemmaeii |
| 901 | svqvpkilsg kvkpiyfhtq |

Androgen receptor, isoform 2 NP_001011645.1 (SEQ ID NO: 181)

| | |
|---|---|
| 1 | milwlhslet ardhvlpidy yfppqktcli cgdeasgchy galtcgsckv ffkraaegkq |
| 61 | kylcasrndc tidkfrrknc pscrlrkcye agmtlgarkl kklgnlklqe egeassttsp |
| 121 | teettqkltv shiegyecqp iflnvleaie pgvvcaghdn nqpdsfaall sslnelgerq |
| 181 | lvhvvkwaka lpgfrnlhvd dqmaviqysw mglmvfamgw rsftnvnsrm lyfapdlvfn |
| 241 | eyrmhksrmy sqcvrmrhls qefgwlqitp qeflcmkall lfsiipvdgl knqkffdelr |
| 301 | mnyikeldri iackrknpts csrrfyqltk lldsvqpiar elhqftfdll ikshmvsvdf |
| 361 | pemmaeiisv qvpkilsgkv kpiyfhtq |

Androgen receptor, isoform 3 NP_001334990.1 (SEQ ID NO: 182)

| | |
|---|---|
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe aysegsssgr areasgapts skdnylggts tisdnakelc |
| 241 | kaysysmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgek frvgnckhlk mtrp |

Androgen receptor, isoform 4 NP_001334992.1 (SEQ ID NO: 183)

| | |
|---|---|
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe aysegsssgr areasgapts skdnylggts tisdnakelc |
| 241 | kaysysmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgaa vvvserilrv fgvsewlp |

Androgen receptor, isoform 5 NP_001334993.1 (SEQ ID NO: 184)

| | |
|---|---|
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe aysegsssgr areasgapts skdnylggts tisdnakelc |
| 241 | kaysysmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrn |
| 541 | trrkrlwkli irsinscics pretevpvrq qk |

ATPase H+ transporting accessory protein 1 NP_001174.2 (SEQ ID NO: 185)

| | |
|---|---|
| 1 | mmaamatarv rmgprcaqal wrmpwlpvfl slaaaaaaaa aeqqvplvlw ssdrdlwapa |
| 61 | adtheghits dlqlstyldp alelgprnvl lflqdklsie dftayggvfg nkqdsafsnl |
| 121 | enaldlapss lvlpavdwya vstlttylqe klgasplhvd latlrelkln aslpalllir |
| 181 | lpytassglm aprevltgnd evigqvlstl ksedvpytaa ltavrpsrva rdvavvaggl |
| 241 | grqllqkqpv spvihppvsy ndtaprilfw aqnfsvaykd qwedltpltf gvqelnitgs |
| 301 | fwndsfarls ltyerlfgtt vtfkfilanr lypvsarhwf tmerlevhsn gsvayfnasq |
| 361 | vtgpsiysfh ceyvsslskk gsllvartqp spwqmmlqdf qiqafnvmge qfsyasdcas |
| 421 | ffspgiwmgl ltslfmlfif tyglhmilsl ktmdrfddhk gptisltqiv |

B melanoma antigen 1 precursor NP_001178.1 (SEQ ID NO: 186)

| | |
|---|---|
| 1 | maaravflal saqllqarlm keespvvswr lepedgtalc fif |

BCR/ABL fusion protein el4ab NG 050673.1 (SEQ ID NO: 187)

| | |
|---|---|
| 1 | gcacctgcag ggagggcagg cagctagcct gaaggctgat ccccccttcc tgttagcact |
| 61 | tttgatggga ctagtggact ttggttcaga aggaagagct atgcttgtta gggcctcttg |
| 121 | tctcctccca ggagtggaca aggtgggtta ggagcagttt ctccctgagt ggctgctgct |
| 181 | gggtggttga ggagatgcac ggcttctgtt cctagtcaca aggctgcagc agacgctcct |
| 241 | cagatgctct gtgccttgga tctggcccca ctcccgtcct cccagccctc ctctcctcca |
| 301 | gctacctgcc agccggcact tttggtcaag ctgttttgca ttcactgttg cacatatgct |
| 361 | cagtcacaca cacagcatac gctatgcaca tgtgtccaca cacaccccac ccacatccca |
| 421 | catcaccccg accccctctg ctgtccttgg aaccttatta cacttcgagt cactggtttg |
| 481 | cctgtattgt gaaaccagct ggatcctgag atccccaaga cagaaatcat gatgagtatg |
| 541 | tttttggccc atgacactgg cttaccttgt gccaggcaga tggcagccac acagtgtcca |
| 601 | ccggatggtt gattttgaag cagagttagc ttgtcacctg cctccctttc ccgggacaac |
| 661 | agaagctgac ctctttgatc tcttgcgcag atgatgagtc tccggggctc tatgggtttc |
| 721 | tgaatgtcat cgtccactca gccactggat ttaagcagag ttcaagtaag tactggtttg |
| 781 | gggaggaggg ttgcagcggc cgagccaggg tctccaccca ggaaggactc atcgggcagg |
| 841 | gtgtggggaa acagggaggt tgttcagatg accacgggac acctttgacc ctggccgctg |
| 901 | tggagtgttt gtgctggttg atgccttctg ggtgtggaat tgtttttccc ggagtggcct |
| 961 | ctgccctctc ccctagcctg tctcagatcc tgggagctgg tgagctgccc cctgcaggtg |
| 1021 | gatcgagtaa ttgcaggggt ttggcaagga ctttgacaga catccccagg ggtgcccggg |
| 1081 | agtgtggggt ccaagccagg agggctgtca gcagtgcacc ttcaccccac agcagagcag |
| 1141 | atttggctgc tctgtcgagc tggatggata ctactttttt tttcctttcc ctctaagtgg |
| 1201 | gggtctcccc cagctactgg agctgtcaga acagtgaagg ctggtaacac atgagttgca |
| 1261 | ctgtgtaagt ttctcgaggc cgggcgcagt ggctcatgcc tgtaatccca gcactttggg |
| 1321 | aggctgaggc aggtggatcg cttgagctca ggagttggag accagcctga ccaacatggt |
| 1381 | gaaaccctgt gtctactaaa aatacaaaga ttagccgggc taggcagtgg gcacctgtaa |
| 1441 | tcacaactgc ttgggaggct gagggaagag aatcgcttga acccaggagg cggaggttgc |
| 1501 | agtgagccga gcttgtgcca ctgcattcca gcctgggcga cagagcaaga ctccgcctca |
| 1561 | aaaaaaaaaa aaaaaagttc ctagaaacag caaaatgtgg agacagaaag cttaccaggg |
| 1621 | attgttgggg aatggggttg ggagagagga ctaactgcag atgaacccaa gggggacttt |
| 1681 | ttaggtgaga gcagtgtcgt gaaaagactg tggtgctgtt tgcgctcaca tttacatttc |
| 1741 | ctaaaattct ttaaacccta cacttggaat ggatgaatta catgacatgc agattgcacc |
| 1801 | ttcataacat aatctttctc ctgggcccct gtctctggct gcctcataaa cgctggtgtt |
| 1861 | tccctcgtgg gcctccctgc atccctgcat ctcctcccgg gtcctgtctg tgagcaatac |
| 1921 | agcgtgacac cctacgctgc cccgtggtcc cgggcttgtc tctccttgcc tccctgttac |
| 1981 | ctttctttct atctcttcct tgccccgtgc actcaacctt gcatccccaa accaaaccta |
| 2041 | ttattcatgg accccaaact tgttcctctt atgtcctgtc cctttgaggg gcaccaccat |
| 2101 | ccacccgcat ggccaagcca gaaaccgtgg tctgctctcc ctccgttaaa tgccattctc |
| 2161 | catcagtgag gcttcttagt catctctggc tgcctggcca ggccctggct gtggcctcct |
| 2221 | ccctggtctt tgtagctctg gatatccctg cagaaagggt ccccactacc aggcctctcc |
| 2281 | atccccagtc tcaggtagtt tttctaaaat gcaaacccca ccctgcaact taccgcccac |
| 2341 | agcccagccc actcttctcc aggcctcgcc tccctccctt ccccctgcac cccacgactt |
| 2401 | ctccagcact gagctgcttc ctgtgcccca cagtggcctg gagtcccctt tgccttaact |
| 2461 | ctttgcccca tagtacagcg gggtctgctc tgattgtagg ggcttcccac atcccccagg |
| 2521 | atggctgccc tctgctgtgg catcactgtg taacaatggc gtgtacacct ctctgtcccc |
| 2581 | accagtgcag ggcccttctc atcgtagggg ctttagctgg ggtttgtgga tcgactgagt |
| 2641 | gaacgaatgt tgtgggaagt cccgtttccc agccgcaccc agggaaattc cacagagcgg |
| 2701 | gcaggggcat cgcatgaggt gctggtgttc acgccagacc acaattaggt gtttaatttt |
| 2761 | taaaaagaaa gttacaacct ttttttttta tttttatttt ttctgattct gcaaataaca |
| 2821 | cctgctctta cagaccatgt gggtgatgtg gaaaagacct gtgaccttct ccatgtccac |
| 2881 | ttctccccac agatctgtac tgcaccctgg aggtggattc ctttgggtat tttgtgaata |
| 2941 | aagcaaagac gcgcgtctac agggacacag ctgagcca |

Serine/threonine-protein kinase B-raf, isoform 1 NP_004324.2 (SEQ ID NO: 188)

| | |
|---|---|
| 1 | maalsggggg gaepgqalfn gdmepeagag agaaassaad paipeevwni kqmikltqeh |
| 61 | iealldkfgg ehnppsiyle ayeeytskld alqqreqqll eslgngtdfs vsssasmdtv |
| 121 | tsssssslsv lpsslsvfqn ptdvarsnpk spqkpivrvf lpnkqrtvvp arcgvtvrds |
| 181 | lkkalmmrgl ipeccavyri qdgekkpigw dtdiswltge elhvevlenv pltthnfvrk |
| 241 | tfftlafcdf crkllfqgfr cqtcgykfhq rcstevplmc vnydqldllf vskffehhpi |
| 301 | pqeeaslaet altsgsspsa pasdsigpqi ltspspsksi pipqpfrpad edhrnqfgqr |
| 361 | drsssapnvh intiepvnid dlirdqgfrg dggsttglsa tppaslpgsl tnvkalqksp |
| 421 | gpqrerksss ssedrnrmkt lgrrdssddw eipdgqitvg qrigsgsfgt vykgkwhgdv |
| 481 | avkmlnvtap tpqqlqafkn evgvlrktrh vnillfmgys tkpqlaivtq wcegsslyhh |
| 541 | lhiietkfem iklidiarqt aqgmdylhak siihrdlksn niflhedltv kigdfglatv |
| 601 | ksrwsgshqf eqlsgsilwm apevirmqdk npysfqsdvy afgivlyelm tgqlpysnin |
| 661 | nrdqiifmvg rgylspdlsk vrsncpkamk rlmaeclkkk rderplfpqi lasiellars |
| 721 | lpkihrsase pslnragfqt edfslyacas pktpiqaggy gafpvh |

Serine/threonine-protein kinase B-raf, isoform 2 NP_001341538.1 (SEQ ID NO:
189)

| | |
|---|---|
| 1 | maalsggggg gaepgqalfn gdmepeagag agaaassaad paipeevwni kqmikltqeh |
| 61 | iealldkfgg ehnppsiyle ayeeytskld alqqreqqll eslgngtdfs vsssasmdtv |
| 121 | tsssssslsv lpsslsvfqn ptdvarsnpk spqkpivrvf lpnkqrtvvp arcgvtvrds |
| 181 | lkkalmmrgl ipeccavyri qdgekkpigw dtdiswltge elhvevlenv pltthnfvrk |
| 241 | tfftlafcdf crkllfqgfr cqtcgykfhq rcstevplmc vnydqldllf vskffehhpi |
| 301 | pqeeaslaet altsgsspsa pasdsigpqi ltspspsksi pipqpfrpad edhrnqfgqr |
| 361 | drsssapnvh intiepvnid dlirdqgfrg dggsttglsa tppaslpgsl tnvkalqksp |
| 421 | gpqrerksss ssedrnrmkt lgrrdssddw eipdgqitvg grigsgsfgt vykgkwhgdv |
| 481 | avkmlnvtap tpqqlqafkn evgvlrktrh vnillfmgys tkpqlaivtq wcegsslyhh |
| 541 | lhiietkfem iklidiarqt aqgmdylhak siihrdlksn niflhedltv kigdfglatv |
| 601 | ksrwsgshqf eqlsgsilwm apevirmqdk npysfqsdvy afgivlyelm tgqlpysnin |
| 661 | nrdqiifmvg rgylspdlsk vrsncpkamk rlmaeclkkk rderplfpqi lasiellars |
| 721 | lpkihrsase pslnragfqt edfslyacas pktpiqaggy gefaafk |

Carbonic anhydrase 9 precursor NP_001207.2 (SEQ ID NO: 190)

| | |
|---|---|
| 1 | maplcpspwl pllipapapg ltvqlllsll llvpvhpqrl prmqedsplg ggssgeddpl |
| 61 | geedlpseed spreedppge edlpgeedlp geedlpevkp kseeegslkl edlptveapg |
| 121 | dpqepqnnah rdkegddqsh wryggdppwp rvspacagrf qspvdirpql aafcpalrpl |
| 181 | ellgfqlppl pelrlrnngh svqltlppgl emalgpgrey ralqlhlhwg aagrpgseht |
| 241 | veghrfpaei hvvhlstafa rvdealgrpg glavlaafle egpeensaye qllsrleeia |
| 301 | eegsetqvpg ldisallpsd fsryfqyegs lttppcaqgv iwtvfnqtvm lsakqlhtls |
| 361 | dtlwgpgdsr lqlnfratqp lngrvieasf pagvdsspra aepvqlnscl aagdilalvf |
| 421 | gllfavtsva flvqmrrqhr rgtkggvsyr paevaetga |

G/mitotic-speqfic cyclin-B1, isoform 1 NP_114172.1 (SEQ ID NO: 191)

| | |
|---|---|
| 1 | malrvtrnsk inaenkakin magakrvpta paatskpglr prtalgdign kvseqlqakm |
| 61 | pmkkeakpsa tgkvidkklp kplekvpmlv pvpvsepvpe pepepepepv keeklspepi |
| 121 | lvdtaspspm etsgcapaee dlcqafsdvi lavndvdaed gadpnlcsey vkdiyaylrq |
| 181 | leeeqavrpk yllgrevtgn mrailidwlv qvqmkfrllq etmymtvsii drfmqnncvp |
| 241 | kkmlqlvgvt amfiaskyee myppeigdfa fvtdntytkh qirqmemkil ralnfglgrp |
| 301 | lplhflrras kigevdveqh tlakylmelt mldydmvhfp psqiaagafc lalkildnge |
| 361 | wtptlqhyls yteesllpvm qhlaknvvmv nqgltkhmtv knkyatskha kistlpqlns |
| 421 | alvqdlakav akv |

G/mitotic-speqfic cyclin-B1, isoform 2 NP_001341773.1 (SEQ ID NO: 192)

| | |
|---|---|
| 1 | malrvtrnsk inaenkakin magakrvpta paatskpglr prtalgdign kvseqlqakm |
| 61 | pmkkeakpsa tgkvidkklp kplekvpmlv pvpvsepvpe pepepepepv keeklspepi |
| 121 | lvdtaspspm etsgcapaee dlcqafsdvi lavndvdaed gadpnlcsey vkdiyaylrq |
| 181 | leeeqavrpk yllgrevtgn mrailidwlv qvqmkfrllq etmymtvsii drfmqnncvp |
| 241 | kkmlqlvgvt amfiaskyee myppeigdfa fvtdntytkh qirqmemkil ralnfglgrp |
| 301 | lplhflrras kigevdveqh tlakylmelt mldydmvhfp psqiaagafc lalkildnge |
| 361 | wtvknkyats khakistlpq lnsalvqdla kavakv |

G/mitotic-speqfic cyclin-B1, isoform 3 NP_001341774.1 (SEQ ID NO: 193)

| | |
|---|---|
| 1 | malrvtrnsk inaenkakin magakrvpta paatskpglr prtalgdign kvseqlqakm |
| 61 | pmkkeakpsa tgkvidkklp kplekvpmlv pvpvsepvpe pepepepepv keeklspepi |
| 121 | lvdtaspspm etsgcapaee dlcqafsdvi lavndvdaed gadpnlcsey vkdiyaylrq |
| 181 | lenncvpkkm lqlvgvtamf iaskyeemyp peigdfafvt dntytkhqir qmemkilral |
| 241 | nfglgrplpl hflrraskig evdveqhtla kylmeltmld ydmvhfppsq iaagafclal |
| 301 | kildngewtp tlqhylsyte esllpvmqhl aknvvmvnqg ltkhmtvknk yatskhakis |
| 361 | tlpqlnsalv qdlakavakv |

CD276, isoform a precursor NP_001019907.1 (SEQ ID NO: 194)

| | |
|---|---|
| 1 | mlrrrgspgm gvhvgaalga lwfcltgale vqvpedpvva lvgtdatlcc sfspepgfsl |
| 61 | aqlnliwqlt dtkqlvhsfa egqdqgsaya nrtalfpdll aqgnaslrlq rvrvadegsf |
| 121 | tcfvsirdfg saayslqvaa pyskpsmtle pnkdlrpgdt vtitcssyqg ypeaevfwqd |
| 181 | gqgvpltgnv ttsqmaneqg lfdvhsilrv vlgangtysc lvrnpvlqqd ahssvtitpq |
| 241 | rsptgavevq vpedpvvalv gtdatlrcsf spepgfslaq lnliwqltdt kqlvhsfteg |
| 301 | rdqgsayanr talfpdllaq gnaslrlqrv rvadegsftc fvsirdfgsa avslqvaapy |
| 361 | skpsmtlepn kdlrpgdtvt itcssyrgyp eaevfwqdgq gvpltgnvtt sqmaneqglf |
| 421 | dvhsvlrvvl gangtysclv rnpvlqqdah gsvtitgqpm tfppealwvt vglsvclial |
| 481 | lvalafvcwr kikqsceeen agaedqdgeg egsktalqpl khsdskeddg qeia |

CD276, isoform b precursor NP_001316557.1, NP_079516.1 (SEQ ID NO: 195)

| | |
|---|---|
| 1 | mlrrrgspgm gvhvgaalga lwfcltgale vqvpedpvva lvgtdatlcc sfspepgfsl |
| 61 | aqlnliwqlt dtkqlvhsfa egqdqgsaya nrtalfpdll aqgnaslrlq rvrvadegsf |
| 121 | tcfvsirdfg saayslqvaa pyskpsmtle pnkdlrpgdt vtitcssyrg ypeaevfwqd |
| 181 | gqgvpltgnv ttsqmaneqg lfdvhsvlrv vlgangtysc lvrnpvlqqd ahgsvtitgq |
| 241 | pmtfppealw vtvglsvcli allvalafvc wrkikqscee enagaedqdg egegsktalq |
| 301 | plkhsdsked dgqeia |

CD276, isoform c NP_001316558.1 (SEQ ID NO: 196)

| | |
|---|---|
| 1 | mtlepnkdlr pgdtvtitcs syqgypeaev fwqdgqgvpl tgnvttsqma neqglfdvhs |
| 61 | ilrvvlgang tysclvrnpv lqqdahssvt itpqrsptga vevqvpedpv valvgtdatl |
| 121 | rcsfspepgf slaqlnliwq ltdtkqlvhs ftegrdqgsa yanrtalfpd llaqgnaslr |
| 181 | lqrvrvadeg sftcfvsird fgsaayslqv aapyskpsmt lepnkdlrpg dtvtitcssy |
| 241 | rgypeaevfw qdgqgvpltg nvttsqmane qglfdvhsvl rvvlgangty sclvrnpvlq |
| 301 | qdahgsvtit gqpmtfppea lwvtvglsvc liallvalaf vcwrkikqsc eeenagaedq |
| 361 | dgegegskta lqplkhsdsk eddgqeia |

Carcinoembryonic antigen-related cell adhesion molecule 3, isoform 1
precursor NP_001806.2 (SEQ ID NO: 197)

| | |
|---|---|
| 1 | mgppsasphr ecipwqglll tasllnfwnp pttaklties mplsvaegke vlllvhnlpq |
| 61 | hlfgyswykg ervdgnsliv gyvigtqqat pgaaysgret iytnaslliq nvtqndigfy |
| 121 | tlqviksdlv neeatgqfhv yqenapglpv gavagivtgv lvgvalvaal vcflllaktg |
| 181 | rtsiqrdlke qqpqalapgr gpshssafsm splstaqapl pnprtaasiy eellkhdtni |
| 241 | ycrmdhkaev as |

Carcinoembryonic antigen-related cell adhesion molecule 3, isoform 2
precursor NP_001264092.1 (SEQ ID NO: 198)

| | |
|---|---|
| 1 | mgppsasphr ecipwqglll tasllnfwnp pttaklties mplsvaegke vlllvhnlpq |
| 61 | hlfgyswykg ervdgnsliv gyvigtqqat pgaaysgret iytnaslliq nvtqndigfy |
| 121 | tlqviksdlv neeatgqfhv yqenapglpv gavagivtgv lvgvalvaal vcflllaktg |
| 181 | rpwslpqlcl ldvpslhcpg pptqpqdssf hl |

Carcinoembryonic antigen-related cell adhesion molecule 5, isoform 1
preprotein NP_001278413.1, NP_004354.3 (SEQ ID NO: 199)

| | |
|---|---|
| 1 | mespsapphr wcipwqrlll taslltfwnp pttaklties tpfnvaegke vlllvhnlpq |
| 61 | hlfgyswykg ervdgnrqii gyvigtqqat pgpaysgrei iypnaslliq niiqndtgfy |
| 121 | tlhviksdlv neeatgqfrv ypelpkpsis snnskpvedk davaftcepe tqdatylwwv |
| 181 | nnqslpvspr lqlsngnrtl tlfnvtrndt asykcetqnp vsarrsdsvi lnvlygpdap |
| 241 | tisplntsyr sgenlnlsch aasnppaqys wfvngtfqqs tqelfipnit vnnsgsytcq |
| 301 | ahnsdtglnr ttvttitvya eppkpfitsn nsnpvededa valtcepeiq nttylwwvnn |
| 361 | qslpvsprlq lsndnrtltl lsvtrndvgp yecgiqnels vdhsdpviln vlygpddpti |
| 421 | spsytyyrpg vnlslschaa snppaqyswl idgniqghtq elfisnitek nsglytcqan |
| 481 | nsasghsrtt vktitvsael pkpsissnns kpvedkdava ftcepeaqnt tylwwvngqs |
| 541 | lpvsprlqls ngnrtltlfn vtrndarayv cgiqnsysan rsdpvtldvl ygpdtpiisp |
| 601 | pdssylsgan lnlschsasn pspqyswrin gipqqhtqvl fiakitpnnn gtyacfvsnl |
| 661 | atgrnnsivk sitvsasgts pglsagatvg imigvlvgva li |

Carcinoembryonic antigen-related cell adhesion molecule 5, isoform 2
preprotein NP_001295327.1 (SEQ ID NO: 200)

| | |
|---|---|
| 1 | mespsapphr wcipwqrlll taslltfwnp pttaklties tpfnvaegke vlllvhnlpq |
| 61 | hlfgyswykg ervdgnrqii gyvigtqqat pgpaysgrei iypnaslliq niiqndtgfy |
| 121 | tlhviksdlv neeatgqfrv ypelpkpsis snnskpvedk davaftcepe tqdatylwwv |
| 181 | nnqslpvspr lqlsngnrtl tlfnvtrndt asykcetqnp vsarrsdsvi lnvlygpdap |
| 241 | tisplntsyr sgenlnlsch aasnppaqys wfvngtfqqs tqelfipnit vnnsgsytcq |
| 301 | ahnsdtglnr ttvttitvye ppkpfitsnn snpvededav altcepeiqn ttylwwvnnq |
| 361 | slpvsprlql sndnrtltll svtrndvgpy ecgiqnelsv dhsdpvilnv lygpddptis |
| 421 | psytyyrpgv nlslschaas nppaqyswli dgniqqhtqe lfisnitekn sglytcqann |
| 481 | sasghsrttv ktitvsaelp kpsissnnsk pvedkdavaf tcepeaqntt ylwwvngqsl |
| 541 | pvsprlqlsn gnrtltlfnv trndarayvc giqnsysanr sdpvtldvly gpdtpiispp |
| 601 | dssylsganl nlschsasnp spqyswring ipqqhtqvlf iakitpnnng tyacfvsnla |
| 661 | tgrnnsivks itvsasgtsp glsagatvgi migvlvgval i |

Baculoviral IAP repeat containing 2, isoform 1 NP_001157.1, NP_001243092.1
(SEQ ID NO: 201)

| | |
|---|---|
| 1 | mhktasqrlf pgpsyqniks imedstilsd wtnsnkqkmk ydfscelyrm stystfpagv |
| 61 | pvserslara gfyytgvndk vkcfccglml dnwklgdspi qkhkqlypsc sfiqnlvsas |
| 121 | lgstskntsp mrnsfahsls ptlehsslfs gsysslspnp lnsravedis ssrtnpysya |
| 181 | msteearflt yhmwpltfls pselaragfy yigpgdrvac facggklsnw epkddamseh |
| 241 | rrhfpncpfl ensletlrfs isnlsmqtha armrtfmywp ssvpvqpeql asagfyyvgr |
| 301 | nddvkcfccd gglrcwesgd dpwvehakwf prceflirmk gqefvdeiqg ryphlleqll |
| 361 | stsdttgeen adppiihfgp gesssedavm mntpvvksal emgfnrdlvk qtvqskiltt |
| 421 | genyktvndi vsallnaede kreeekekqa eemasddlsl irknrmalfq qltcvlpild |
| 481 | nllkanvink qehdiikqkt qiplqareli dtilvkgnaa anifknclke idstlyknlf |
| 541 | vdknmkyipt edvsglslee qlrrlqeert ckvcmdkevs vvfipcghlv vcqecapslr |
| 601 | kcpicrgiik gtvrtfls |

Baculoviral IAP repeat containing 2, isoform 2 NP_001243095.1 (SEQ ID NO:
202)

| | |
|---|---|
| 1 | mstystfpag vpvserslar agfyytgvnd kvkcfccglm ldnwklgdsp iqkhkqlyps |
| 61 | csfiqnlvsa slgstsknts pmrnsfahsl sptlehsslf sgsysslspn pinsravedi |
| 121 | sssrtnpysy amsteearfl tyhmwpltfl spselaragf yyigpgdrva cfacggklsn |
| 181 | wepkddamse hrrhfpncpf lensletlrf sisnlsmqth aarmrtfmyw pssvpvqpeq |
| 241 | lasagfyyvg rnddvkcfcc dgglrcwesg ddpwvehakw fprceflirm kgqefvdeiq |
| 301 | gryphlleql lstsdttgee nadppiihfg pgesssedav mmntpvvksa lemgfnrdlv |
| 361 | kqtvqskilt tgenyktvnd ivsallnaed ekreeekekq aeemasddls lirknrmalf |
| 421 | qqltcvlpil dnllkanvin kqehdiikqk tqiplqarel idtilvkgna aanifknclk |
| 481 | eidstlyknl fvdknmkyip tedvsglsle eqlrrlqeer tckvcmdkev svvfipcghl |
| 541 | vvcqecapsl rkcpicrgii kgtvrtfls |

Chondrosarcoma-associated gene 2/3 protein, isoform X1 XP 006724920.1 (SEQ ID
NO: 203)

| | |
|---|---|
| 1 | mwmgliqlve gvkrkdqgfl ekefyhktni kmrceflacw paftvlgeaw rdqvdwsrll |
| 61 | rdtglvkmsr kprassplsn nhpptpkrrg sgrhpinpgp ealskfprqp grekgpikev |
| 121 | pgtkgsp |

Chondrosarcoma-associated gene 2/3 protein, isoform X2 XP 016885512.1 (SEQ ID
NO: 204)

| | |
|---|---|
| 1 | mwmgliqlve gvkrkdqgfl ekefyhktni kmrceflacw paftvlgeaw rdqvdwsrll |
| 61 | rdtglvkmsr kprassplsn nhpptpkrfp rqpgrekgpi kevpgtkgsp |

Chondroitin sulfate proteoglycan 4 precursor NP_001888.2 (SEQ ID NO: 205)

| | |
|---|---|
| 1 | mqsgprpplp apglalaltl tmlarlasaa sffgenhlev pvataltdid lqlqfstsqp |
| 61 | eallllaagp adhlllqlys grlqvrlvlg qeelrlqtpa etllsdsiph tvvltvvegw |
| 121 | atlsvdgfln assavpgapl evpyglfvgg tgtlglpylr gtsrplrgcl haatlngrsl |
| 181 | lrpltpdvhe gcaeefsasd dvalgfsgph slaafpawgt qdegtleftl ttqsrqapla |
| 241 | fqaggrrgdf iyvdifeghl ravvekgqgt vllhnsvpva dgqphevsvh inahrleisv |
| 301 | dqypthtsnr gvlsyleprg slllggldae asrhlqehrl gltpeatnas llgcmedlsv |
| 361 | ngqrrglrea lltrnmaagc rleeeeyedd ayghyeafst lapeawpame lpepcvpepg |
| 421 | lppvfanftq lltisplvva eggtawlewr hvqptldlme aelrksqvlf svtrgarhge |
| 481 | leldipgaqa rkmftlldvv nrkarfihdg sedtsdqlvl evsvtarvpm psclrrgqty |
| 541 | llpiqvnpvn dpphiifphg slmvilehtq kplgpevfqa ydpdsacegl tfqvlgtssg |
| 601 | lpverrdqpg epatefscre leagslvyvh rggpaqdltf rvsdglqasp patlkvvair |
| 661 | paiqihrstg lrlaqgsamp ilpanlsvet navgqdvsvl frvtgalqfg elqkqgaggv |
| 721 | egaewwatqa fhqrdveqgr vrylstdpqh haydtvenla levqvgqeil snlsfpvtiq |
| 781 | ratvwmlrle plhtqntqqe tlttahleat leeagpsppt fhyevvqapr kgnlqlqgtr |
| 841 | lsdgqgftqd diqagrvtyg ataraseave dtfrfrvtap pyfsplytfp ihiggdpdap |
| 901 | vltnvllvvp eggegvlsad hlfvkslnsa sylyevmerp rhgrlawrgt qdkttmvtsf |
| 961 | tnedllrgrl vyqhddsett eddipfvatr qgessgdmaw eevrgvfrva iqpvndhapv |
| 1021 | qtisrifhva rggrrllttd dvafsdadsg fadaqlvltr kdllfgsiva vdeptrpiyr |
| 1081 | ftqedlrkrr vlfvhsgadr gwiqlqvsdg qhqatallev qasepylrva ngsslvvpqg |
| 1141 | gqgtidtavl hldtnldirs gdevhyhvta gprwgqlvra gqpatafsqq dlldgavlys |
| 1201 | hngslsprdt mafsveagpv htdatlqvti alegplaplk lvrhkkiyvf qgeaaeirrd |
| 1261 | qleaaqeavp padivfsvks ppsagylvmv srgaladepp sldpvqsfsq eavdtgrvly |
| 1321 | lhsrpeawsd afsldvasgl gaplegvlve levlpaaipl eaqnfsvpeg gsltlappll |
| 1381 | rvsgpyfptl lglslqvlep pqhgalqked gpqartlsaf swrmveeqli ryvhdgsetl |
| 1441 | tdsfvlmana semdrqshpv aftvtvlpvn dqppilttnt glqmwegata pipaealrst |
| 1501 | dgdsgsedlv ytieqpsngr vvlrgapgte vrsftqaqld gglvlfshrg tldggfrfrl |
| 1561 | sdgehtspgh ffrvtaqkqv llslkgsqtl tvcpgsvqpl ssqtlrasss agtdpqllly |
| 1621 | rvvrgpqlgr lfhaqqdstg ealvnftqae vyagnilyeh emppepfwea hdtlelqlss |
| 1681 | ppardvaatl avavsfeaac pqrpshlwkn kglwvpegqr aritvaalda snllasvpsp |
| 1741 | qrsehdvlfq vtqfpsrgql lvseeplhag qphflqsqla agqlvyahgg ggtqqdgfhf |
| 1801 | rahlqgpaga svagpqtsea faitvrdvne rppqpqasvp lrltrgsrap israqlsvvd |
| 1861 | pdsapgeiey evqraphngf lslvggglgp vtrftqadvd sgrlafvang ssvagifqls |
| 1921 | msdgaspplp mslavdilps aievqlrapl evpqalgrss lsqqqlrvvs dreepeaayr |
| 1981 | liqgpqyghl lvggrptsaf sqfqidggev vfaftnfsss hdhfrvlala rgvnasavvn |
| 2041 | vtvrallhvw aggpwpqgat lrldptvlda gelanrtgsv prfrllegpr hgrvvrvpra |
| 2101 | rtepggsqlv eqftqqdled grlglevgrp egrapgpagd sltlelwaqg vppavasldf |
| 2161 | atepynaarp ysvallsvpe aarteagkpe sstptgepgp masspepava kggflsflea |
| 2221 | nmfsviipmc lvllllalil pllfylrkrn ktgkhdvqvl takprnglag dtetfrkvep |
| 2281 | gqaipltavp gqgpppggqp dpellqfcrt pnpalkngqy wv |

Cancer/testis antigen 2 isoform LAGE-1a NP_758965.2 (SEQ ID NO: 206)

| | |
|---|---|
| 1 | mqaegrgtgg stgdadgpgg pgipdgpggn aggpgeagat ggrgprgaga arasgprgga |
| 61 | prgphggaas aqdgrcpcga rrpdsrllel hitmpfsspm eaelvrrils rdaaplprpg |
| 121 | avlkdftvsg nllfirltaa dhrqlqlsis sclqqlsllm witqcflpvf laqapsgqrr |

Cancer/testis antigen 2 isoform LAGE-1b NP_066274.2 (SEQ ID NO: 207)

| | |
|---|---|
| 1 | mqaegrgtgg stgdadgpgg pgipdgpggn aggpgeagat ggrgprgaga arasgprgga |
| 61 | prgphggaas aqdgrcpcga rrpdsrllel hitmpfsspm eaelvrrils rdaaplprpg |
| 121 | avlkdftvsg nllfmsvrdq dregagrmry vgwglgsasp egqkardlrt pkhkvseqrp |
| 181 | gtpgppppeg aqgdgcrgva fnvmfsaphi |

Transcriptional repressor CTCFL, isoform 1 NP_001255969.1, NP_001255970.1,
NP_542185.2 (SEQ ID NO: 208)

| | |
|---|---|
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwin lhrhsekcgs geaksaasgk grrtrkrkqt ilkeatkgqk |
| 601 | eaakgwkeaa ngdeaaaeea sttkgeqfpg emfpvacret tarvkeevde gvtcemllnt |
| 661 | mdk |

Transcriptional repressor CTCFL, isoform 2 NP_001255971.1 (SEQ ID NO: 209)

| | |
|---|---|
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwin lhrhsekcgs geaksaasgk grrtrkrkqt ilkeatkgqk |
| 601 | eaakgwkeaa ngdaaaeeas ttkgeqfpge mfpvacrett arvkeevdeg vtcemllntm |
| 661 | dk |

Transcriptional repressor CTCFL, isoform 3 NP_001255972.1 (SEQ ID NO: 210)

| | |
|---|---|
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwin lhrhsekcgs geaksaasgk grrtrkrkqt ilkeatkgqk |
| 601 | eaakgwkeaa ngdeaaaeea sttkgeqfpg emfpvacret tarvkeevde gvtcemllnt |
| 661 | mdnsagctgr mmlvsawllg rpqetynqgr rrrgsrrvtw |

Transcriptional repressor CTCFL, isoform 4 NP_001255973.1 (SEQ ID NO: 211)

| | |
|---|---|
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwin lhrhsekcgs geaksaasgk grrtrkrkqt ilkeatkgqk |
| 601 | eaakgwkeaa ngdgvisahr nlcllgssds hasysgagit darhhawliv llflvemgfy |
| 661 | hvshs |

Transcriptional repressor CTCFL, isoform 5 NP_001255974.1 (SEQ ID NO: 212)

| | |
|---|---|
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwil wvgnsevael ggpgsgpllr lqsgcppglh hpkaglgped |
| 601 | plpgqlrhtt agtglssllq gplcraa |

Transcriptional repressor CTCFL, isoform 6 NP_001255975.1 (SEQ ID NO: 213)

| | |
|---|---|
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgvhmrnl haysaaelkc rycsavfher |
| 421 | yaliqhqkth knekrfkckh csyackqerh mtahirthtg ekpftclscn kcfrqkqlln |
| 481 | ahfrkyhdan fiptvykcsk cgkgfsrwin lhrhsekcgs geaksaasgk grrtrkrkqt |
| 541 | ilkeatkgqk eaakgwkeaa ngdeaaaeea sttkgeqfpg emfpvacret tarvkeevde |
| 601 | gvtcemllnt mdk |

Transcriptional repressor CTCFL, isoform 7 NP_001255976.1 (SEQ ID NO: 214)

| | |
|---|---|
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveasklkrh |
| 361 | vrshtgerpf qccqcsyasr dtyklkrhmr thsgekpyec hichtrftqs gtmkihilqk |
| 421 | hgenvpkyqc phcatiiark sdlrvhmrnl haysaaelkc rycsavfher yaliqhqkth |
| 481 | knekrfkckh csyackqerh mtahirthtg ekpftclscn kcfrqkqlln ahfrkyhdan |
| 541 | fiptvykcsk cgkgfsrwit skwsglkpqt fit |

Transcriptional repressor CTCFL, isoform 8 NP_001255977.1 (SEQ ID NO: 215)

| | |
|---|---|
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya sveerhmtah |
| 361 | irthtgekpf tclscnkcfr qkqllnahfr kyhdanfipt vykcskcgkg fsrwilwvgn |
| 421 | sevaelggpg sgpllrlqsg cppglhhpka glgpedplpg qlrhttagtg lssllqgplc |
| 481 | raa |

Transcriptional repressor CTCFL, isoform 9 NP_001255978.1 (SEQ ID NO: 216)

| | |
|---|---|
| 1 | msgdersdei vltvsnsnve eqedqotagq adaekakstk nqrktkgakg tfhcdvcmft |
| 61 | ssrmssfnrh mkthtsekph lchlclktfr tvtllrnhvn thtgtrpykc ndcnmafvts |
| 121 | gelvrhrryk hthekpfkcs mckyasveas klkrhvrsht gerpfqccqc syasrdtykl |
| 181 | krhmrthsge kpyechicht rftqsgtmki hilqkhgenv pkyqcphcat iiarksdlrv |
| 241 | hmrnlhaysa aelkcrycsa vfheryaliq hqkthknekr fkckhcsyac kqerhmtahi |
| 301 | rthtgekpft clscnkcfrq kqllnahfrk yhdanfiptv ykcskcgkgf srwinlhrhs |
| 361 | ekcgsgeaks aasgkgrrtr krkqtilkea tkgqkeaakg wkeaangdgv isahrnlcll |
| 421 | gssdshasvs gagitdarhh awlivllflv emgfyhvshs |

Transcriptional repressor CTCFL, isoform 10 NP_001255979.1 (SEQ ID NO: 217)

| | |
|---|---|
| 1 | msgdersdei vltvsnsnve eqedqptagq adaekakstk nqrktkgakg tfhcdvcmft |
| 61 | ssrmssfnrh mkthtsekph lchlclktfr tvtllrnhvn thtgtrpykc ndcnmafvts |
| 121 | gelvrhrryk hthekpfkcs mckyasveas klkrhvrsht gerpfqccqc syasrdtykl |
| 181 | krhmrthsge kpyechicht rftqsgtmki hilqkhgenv pkyqcphcat iiarksdlrv |
| 241 | hmrnlhaysa aelkcrycsa vfheryaliq hqkthknekr fkckhcsyac kqerhmtahi |
| 301 | rthtgekpft clscnkcfrq kqllnahfrk yhdanfiptv ykcskcgkgf srwilwvgns |
| 361 | evaelggpgs gpllrlqsgc ppglhhpkag lgpedplpgq lrhttagtgl ssllqgplcr |
| 421 | aa |

Transcriptional repressor CTCFL, isoform 11 NP_001255980.1, NP_001255981.1
(SEQ ID NO: 218)

| | |
|---|---|
| 1 | maateisvls eqftkikele lmpekglkee ekdgvcrekd hrspseleae rtsgafqdsv |
| 61 | leeevelvla pseesekyil tlqtvhftse avelqdmsll siqqqegvqv vvqqpgpgll |
| 121 | wleegprqsl qqcvaisiqq elyspqemev lqfhaleenv mvasedskla vslaettgli |
| 181 | kleeeqeknq llaertkeql ffvetmsgde rsdeivltvs nsnveeqedq ptagqadaek |
| 241 | akstknqrkt kgakgtfhcd vcmftssrms sfnrhmktht sekphlchlc lktfrtvtll |
| 301 | rnhvnthtgt rpykcndcnm afvtsgelvr hrrykhthek pfkcsmckya svevkpfldl |
| 361 | klhgilveaa vqvtpsvtns ricykqafyy sykiyagnnm hsll |

Transcriptional repressor CTCFL, isoform 12 NP_001255983.1 (SEQ ID NO: 219)

| | |
|---|---|
| 1 | mftssrmssf nrhmkthtse kphlchlclk tfrtvtllrn hvnthtgtrp ykcndcnmaf |
| 61 | vtsgelvrhr rykhthekpf kcsmckyasv easklkrhvr shtgerpfqc cqcsyasrdt |
| 121 | yklkrhmrth sgekpyechi chtrftqsgt mkihilqkhg envpkyqcph catiiarksd |
| 181 | lrvhmrnlha ysaaelkcry csavfherya liqhqkthkn ekrfkckhcs yackqerhmt |
| 241 | ahirthtgek pftclscnkc frqkqllnah frkyhdanfi ptvykcskcg kgfsrwinlh |
| 301 | rhsekcgsge aksaasgkgr rtrkrkqtil keatkgqkea akgwkeaang dgvisahrnl |
| 361 | cllgssdsha sysgagitda rhhawlivll flvemgfyhv shs |

Transcriptional repressor CTCFL, isoform 13 NP_001255984.1 (SEQ ID NO: 220)

| | |
|---|---|
| 1 | mftssrmssf nrhmkthtse kphlchlclk tfrtvtllrn hvnthtgtrp ykcndcnmaf |
| 61 | vtsgelvrhr rykhthekpf kcsmckyasv easklkrhvr shtgerpfqc cqcsyasrdt |
| 121 | yklkrhmrth sgekpyechi chtrftqsgt mkihilqkhg envpkyqcph catiiarksd |
| 181 | lrvhmrnlha ysaaelkcry csavfherya liqhqkthkn ekrfkckhcs yackqerhmt |
| 241 | ahirthtgek pftclscnkc frqkqllnah frkyhdanfi ptvykcskcg kgfsrwvly |

Cytochrome P450 1B1 NP_000095.2 (SEQ ID NO: 221)

| | |
|---|---|
| 1 | mgtslspndp wpinplsiqq ttlllllsvl atvhvgqrll rqrrrqlrsa ppgpfawpli |
| 61 | gnaaavgqaa hlsfarlarr ygdvfqirlg scpivvlnge raihqalvqq gsafadrpaf |
| 121 | asfrvvsggr smafghyseh wkvqrraahs mmrnfftrqp rsrqvleghv lsearelval |
| 181 | lvrgsadgaf ldprpltvva vanvmsavcf gcryshddpe frellshnee fgrtvgagsl |
| 241 | vdvmpwlqyf pnpvrtvfre feqlnrnfsn fildkflrhc eslrpgaapr dmmdafilsa |
| 301 | ekkaagdshg ggarldlenv patitdifga sqdtlstalq wllllftryp dvqtrvqael |
| 361 | dqvvgrdrlp cmgdqpnlpy vlaflyeamr fssfvpvtip hattantsvl gyhipkdtvv |
| 421 | fvnqwsvnhd plkwpnpenf dparfldkdg linkdltsrv mifsvgkrrc igeelskmql |
| 481 | flfisilahq cdfranpnep akmnfsyglt ikpksfkvnv tlresmelld savqnlqake |
| 541 | tcq |

Epidermal growth factor receptor, isoform a precursor NP_005219.2 (SEQ ID NO:
222)

| | |
|---|---|
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs rgrecvdkcn |
| 541 | llegeprefv enseciqchp eclpqamnit ctgrgpdnci qcahyidgph cvktcpagvm |
| 601 | genntlvwky adaghvchlc hpnctygctg pglegcptng pkipsiatgm vgalllllvv |
| 661 | algiglfmrr rhivrkrtlr rllqerelve pltpsgeapn qallrilket efkkikvlgs |
| 721 | gafgtvykgl wipegekvki pvaikelrea tspkankeil deayvmasvd nphvcrllgi |
| 781 | cltstvqlit qlmpfgclld yvrehkdnig sqyllnwcvq iakgmnyled rrlvhrdlaa |
| 841 | rnvlvktpqh vkitdfglak llgaeekeyh aeggkvpikw malesilhri ythqsdvwsy |
| 901 | gvtvwelmtf gskpydgipa seissilekg erlpqppict idvymimvkc wmidadsrpk |
| 961 | freliiefsk mardpqrylv iqgdermhlp sptdsnfyra lmdeedmddv vdadeylipq |
| 1021 | qgffsspsts rtpllsslsa tsnnstvaci drnglqscpi kedsflqrys sdptgalted |
| 1081 | siddtflpvp eyinqsvpkr pagsvqnpvy hnqplnpaps rdphyqdphs tavgnpeyln |
| 1141 | tvqptcvnst fdspahwaqk gshqisldnp dyqqdffpke akpngifkgs taenaeylry |
| 1201 | apqssefiga |

Epidermal growth factor receptor, isoform b precursor NP_958439.1 (SEQ ID NO:
223)

| | |
|---|---|
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs rgrecvdkcn |
| 541 | llegeprefv enseciqchp eclpqamnit ctgrgpdnci qcahyidgph cvktcpagvm |
| 601 | genntlvwky adaghvchlc hpnctygs |

Epidermal growth factor receptor, isoform c precursor NP_958440.1 (SEQ ID NO:
224)

| | |
|---|---|
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gporkvcngi gigefkdsls inatnikhfk |
| 361 | nctsisgdlh ilpvafrgds fthtppldpq eldilktvke itgls |

Epidermal growth factor receptor, isoform d precursor NP_958441.1 (SEQ ID NO:
225)

| | |
|---|---|
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs rgrecvdkcn |
| 541 | llegeprefv enseciqchp eclpqamnit ctgrgpdnci qcahyidgph cvktcpagvm |
| 601 | genntlvwky adaghvchlc hpnctygpgn eslkamlfcl fklsscnqsn dgsyshqsgs |
| 661 | paaqesclgw ipsllpsefq lgwggcshlh awpsasviit assch |

Epidermal growth factor receptor, isoform e precursor NP_001333826.1 (SEQ ID
NO: 226)

| | |
|---|---|
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qgqkcdpscp ngscwgagee ncqkltkiic aqqcsgrcrg |
| 181 | kspsdcchnq caagctgpre sdclvcrkfr deatckdtcp plmlynptty qmdvnpegky |
| 241 | sfgatcvkkc prnyvvtdhg scvracgads yemeedgvrk ckkcegperk vcngigigef |
| 301 | kdslsinatn ikhfknctsi sgdlhilpva frgdsfthtp pldpqeldil ktvkeitgfl |
| 361 | liqawpenrt dlhafenlei irgrtkqhgq fslavvslni tslglrslke isdgdviisg |
| 421 | nknlcyanti nwkklfgtsg qktkiisnrg ensckatgqv chalcspegc wgpeprdcvs |
| 481 | crnvsrgrec vdkcnllege prefvensec iqchpeclpq amnitctgrg pdnciqcahy |
| 541 | idgphcvktc pagvmgennt lvwkyadagh vchlchpnct ygctgpgleg cptngpkips |
| 601 | iatgmvgall lllvvalgig lfmrrrhivr krtlrrllqe relvepltps geapnqallr |
| 661 | ilketefkki kvlgsgafgt vykglwipeg ekvkipvaik elreatspka nkeildeayv |
| 721 | masvdnphvc rllgicltst vqlitqlmpf gclldyvreh kdnigsqyll nwcvqiakgm |
| 781 | nyledrrlvh rdlaarnvlv ktpqhvkitd fglakllgae ekeyhaeggk vpikwmales |
| 841 | ilhriythqs dvwsygvtvw elmtfgskpy dgipaseiss ilekgerlpq ppictidvym |
| 901 | imvkcwmida dsrpkfreli iefskmardp qrylviqgde rmhlpsptds nfyralmdee |
| 961 | dmddvvdade ylipqqgffs spstsrtpll sslsatsnns tvacidrngl qscpikedsf |
| 1021 | lqryssdptg altedsiddt flpvpgewlv wkqscsstss thsaaaslqc psqvlppasp |
| 1081 | egetvadlqt q |

Epidermal growth factor receptor, isoform f precursor NP_001333827.1 (SEQ ID
NO: 227)

| | |
|---|---|
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs rgrecvdkcn |
| 541 | llegeprefv enseciqchp eclpqamnit ctgrgpdnci qcahyidgph cvktcpagvm |
| 601 | genntlvwky adaghvchlc hpnctygctg pglegcptng pkipsiatgm vgalllllvv |
| 661 | algiglfmrr rhivrkrtlr rllqerelve pltpsgeapn qallrilket efkkikvlgs |
| 721 | gafgtvykgl wipegekvki pvaikelrea tspkankeil deayvmasvd nphvcrllgi |
| 781 | cltstvqlit qlmpfgclld yvrehkdnig sqyllnwcvq iakgmnyled rrlvhrdlaa |
| 841 | rnvlvktpqh vkitdfglak llgaeekeyh aeggkvpikw malesilhri ythqsdvwsy |
| 901 | gvtvwelmtf gskpydgipa seissilekg erlpqppict idvymimvkc wmidadsrpk |
| 961 | freliiefsk mardpqrylv iqgdermhlp sptdsnfyra lmdeedmddv vdadeylipq |
| 1021 | qgffsspsts rtpllsslsa tsnnstvaci drnglqscpi kedsflqrys sdptgalted |
| 1081 | siddtflpvp gewlvwkqsc sstssthsaa aslqcpsqvl ppaspegetv adlqtq |

Epidermal growth factor receptor, isoform g precursor NP_001333828.1 (SEQ ID
NO: 228)

| | |
|---|---|
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qgqkcdpscp ngscwgagee ncqkltkiic aqqcsgrcrg |
| 181 | kspsdcchnq caagctgpre sdclvcrkfr deatckdtcp plmlynptty qmdvnpegky |
| 241 | sfgatcvkkc prnyvvtdhg scvracgads yemeedgvrk ckkcegperk vcngigigef |
| 301 | kdslsinatn ikhfknctsi sgdlhilpva frgdsfthtp pldpqeldil ktvkeitgfl |
| 361 | liqawpenrt dlhafenlei irgrtkqhgq fslavvslni tslglrslke isdgdviisg |
| 421 | nknlcyanti nwkklfgtsg qktkiisnrg ensckatgqv chalcspegc wgpeprdcvs |
| 481 | crnvsrgrec vdkcnllege prefvensec iqchpeclpq amnitctgrg pdnciqcahy |
| 541 | idgphcvktc pagvmgennt lvwkyadagh vchlchpnct ygctgpgleg cptngpkips |
| 601 | iatgmvgall lllvvalgig lfmrrrhivr krtlrrllqe relvepltps geapnqallr |
| 661 | ilketefkki kvlgsgafgt vykglwipeg ekvkipvaik elreatspka nkeildeayv |
| 721 | masvdnphvc rllgicltst vqlitqlmpf gclldyvreh kdnigsqyll nwcvqiakgm |
| 781 | nyledrrlvh rdlaarnvlv ktpqhvkitd fglakllgae ekeyhaeggk vpikwmales |
| 841 | ilhriythqs dvwsygvtvw elmtfgskpy dgipaseiss ilekgerlpq ppictidvym |
| 901 | imvkcwmida dsrpkfreli iefskmardp qrylviqgde rmhlpsptds nfyralmdee |
| 961 | dmddvvdade ylipqqgffs spstsrtpll sslsatsnns tvacidrngl qscpikedsf |
| 1021 | lqryssdptg altedsiddt flpvpeyinq svpkrpagsv qnpvyhnqpl npapsrdphy |
| 1081 | qdphstavgn peylntvqpt cvnstfdspa hwaqkgshqi sldnpdyqqd ffpkeakpng |
| 1141 | ifkgstaena eylrvapqss efiga |

Epidermal growth factor receptor, isoform h NP_001333829.1 (SEQ ID NO: 229)

| | |
|---|---|
| 1 | mfnncevvlg nleityvqrn ydlsflktiq evagyvlial ntveriplen lqiirgnmyy |
| 61 | ensyalavls nydanktglk elpmrnlqei lhgavrfsnn palcnvesiq wrdivssdfl |
| 121 | snmsmdfqnh lgscqkcdps cpngscwgag eencqkltki icaqqcsgrc rgkspsdcch |
| 181 | nqcaagctgp resdclvcrk frdeatckdt cpplmlynpt tyqmdvnpeg kysfgatcvk |
| 241 | kcprnyvvtd hgscvracga dsyemeedgv rkckkcegpc rkvcngigig efkdslsina |
| 301 | tnikhfknct sisgdlhilp vafrgdsfth tppldpqeld ilktvkeitg flliqawpen |
| 361 | rtdlhafenl eiirgrtkqh gqfslavvsl nitslglrsl keisdgdvii sgnknlcyan |
| 421 | tinwkklfgt sgqktkiisn rgensckatg qvchalcspe gcwgpeprdc vscrnvsrgr |
| 481 | ecvdkcnlle geprefvens eciqchpecl pqamnitctg rgpdncigca hyidgphcvk |
| 541 | tcpagvmgen ntivwkyada ghvchlchpn ctygctgpgl egcptngpki psiatgmvga |
| 601 | lllllvvalg iglfmrrrhi vrkrtlrrll qerelveplt psgeapnqal lrilketefk |
| 661 | kikvlgsgaf gtvykglwip egekvkipva ikelreatsp kankeildea yvmasvdnph |
| 721 | vcrllgiclt stvqlitqlm pfgclldyvr ehkdnigsqy llnwcvqiak gmnyledrrl |
| 781 | vhrdlaarnv lvktpqhvki tdfglakllg aeekeyhaeg gkvpikwmal esilhriyth |
| 841 | qsdvwsygvt vwelmtfgsk pydgipasei ssilekgerl pqppictidv ymimvkcwmi |
| 901 | dadsrpkfre liiefskmar dpqrylviqg dermhlpspt dsnfyralmd eedmddvvda |
| 961 | deylipqqgf fsspstsrtp llsslsatsn nstvacidrn glqscpiked sflqryssdp |
| 1021 | tgaltedsid dtflpvpeyi nqsvpkrpag svqnpvyhnq pinpapsrdp hyqdphstav |
| 1081 | gnpeylntvq ptcvnstfds pahwaqkgsh qisldnpdyq qdffpkeakp ngifkgstae |
| 1141 | naeylrvapq ssefiga |

Epidermal growth factor receptor, isoform i precursor NP_001333870.1 (SEQ ID
NO: 230)

| | |
|---|---|
| 1 | mrpsgtagaa llallaalcp asraleekkg nyvvtdhgsc vracgadsye meedgvrkck |
| 61 | kcegperkvc ngigigefkd slsinatnik hfknctsisg dlhilpvafr gdsfthtppl |
| 121 | dpqeldilkt vkeitgflli qawpenrtdl hafenleiir grtkqhgqfs lavvslnits |
| 181 | lglrslkeis dgdviisgnk nlcyantinw kklfgtsgqk tkiisnrgen sckatgqvch |
| 241 | alcspegcwg peprdcvscr nvsrgrecvd kcnllegepr efvenseciq chpeclpqam |
| 301 | nitctgrgpd nciqcahyid gphcvktcpa gvmgenntlv wkyadaghvc hlchpnctyg |
| 361 | ctgpglegcp tngpkipsia tgmvgallll lvvalgiglf mrrrhivrkr tlrrllqere |
| 421 | lvepltpsge apnqallril ketefkkikv lgsgafgtvy kglwipegek vkipvaikel |
| 481 | reatspkank eildeayvma svdnphvcrl lgicltstvq litqlmpfgc lldyvrehkd |
| 541 | nigsqyllnw cvqiakgmny ledrrlvhrd laarnvlvkt pqhvkitdfg lakllgaeek |
| 601 | eyhaeggkvp ikwmalesil hriythqsdv wsygvtvwel mtfgskpydg ipaseissil |
| 661 | ekgerlpqpp ictidvymim vkcwmidads rpkfreliie fskmardpqr ylviqgderm |
| 721 | hlpsptdsnf yralmdeedm ddvvdadeyl ipqqgffssp stsrtpllss lsatsnnstv |
| 781 | acidrnglqs cpikedsflq ryssdptgal tedsiddtfl pvpeyinqsv pkrpagsvqn |
| 841 | pvyhnqplnp apsrdphyqd phstavgnpe ylntvqptcv nstfdspahw aqkgshqisl |
| 901 | dnpdyqqdff pkeakpngif kgstaenaey lrvapqssef iga |

Epithelial cell adhesion molecule NP_002345.2 (SEQ ID NO: 231)

| | |
|---|---|
| 1 | mappqvlafg lllaaatatf aaaqeecvce nyklavncfv nnnrqcqcts vgaqntvics |
| 61 | klaakclvmk aemngsklgr rakpegalqn ndglydpdcd esglfkakqc ngtsmcwcvn |
| 121 | tagvrrtdkd teitcservr tywiiielkh karekpydsk slrtalqkei ttryqldpkf |
| 181 | itsilyennv itidlvqnss qktqndvdia dvayyfekdv kgeslfhskk mdltvngeql |
| 241 | dldpgqtliy yvdekapefs mqglkagvia vivvvviavv agivvlvisr kkrmakyeka |
| 301 | eikemgemhr elna |

Ephrin type-A receptor 2, isoform 1 precursor NP_004422.2 (SEQ ID NO: 232)

| | |
|---|---|
| 1 | melqaaracf allwgcalaa aaaaqgkevv lldfaaagge lgwlthpygk gwdlmqnimn |
| 61 | dmpiymysvc nvmsgdqdnw lrtnwvyrge aerifielkf tvrdcnsfpg gasscketfn |
| 121 | lyyaesdldy gtnfqkrlft kidtiapdei tvssdfearh vklnveersv gpltrkgfyl |
| 181 | afqdigacva llsvrvyykk cpellqglah fpetiagsda pslatvagtc vdhavvppgg |
| 241 | eeprmhcavd gewlvpigqc lcqagyekve dacqacspgf fkfeasespc lecpehtlps |
| 301 | pegatscece egffrapqdp asmpctrpps aphyltavgm gakvelrwtp pqdsggredi |
| 361 | vysvtceqcw pesgecgpce asvrysepph gltrtsvtvs dlephmnytf tvearngvsg |
| 421 | lvtsrsfrta sysinqtepp kvrlegrstt slsyswsipp pqqsrvwkye vtyrkkgdsn |
| 481 | synvrrtegf svtlddlapd ttylvqvqal tqegqgagsk vhefqtlspe gsgnlavigg |
| 541 | vavgvvlllv lagvgffihr rrknqrarqs pedvyfskse qlkplktyvd phtyedpnqa |
| 601 | vlkftteihp scvtrqkvig agefgevykg mlktssgkke vpvaiktlka gytekqrvdf |
| 661 | lgeagimgqf shhniirleg viskykpmmi iteymengal dkflrekdge fsvlqlvgml |
| 721 | rgiaagmkyl anmnyvhrdl aarnilvnsn lvckvsdfgl srvleddpea tyttsggkip |
| 781 | irwtapeais yrkftsasdv wsfgivmwev mtygerpywe lsnhevmkai ndgfrlptpm |
| 841 | dcpsaiyqlm mqcwqqerar rpkfadivsi ldklirapds lktladfdpr vsirlpstsg |
| 901 | segvpfrtvs ewlesikmqq ytehfmaagy taiekvvqmt nddikrigvr lpghqkriay |
| 961 | sllglkdqvn tvgipi |

Ephrin type-A receptor 2, isoform 2 NP_001316019.1 (SEQ ID NO: 233)

| | |
|---|---|
| 1 | mqnimndmpi ymysvcnvms gdqdnwlrtn wvyrgeaeri fielkftvrd cnsfpggass |
| 61 | cketfnlyya esdldygtnf qkrlftkidt iapdeitvss dfearhvkln veersvgplt |
| 121 | rkgfylafqd igacvallsv rvyykkcpel lqglahfpet iagsdapsla tvagtcvdha |
| 181 | vvppggeepr mhcavdgewl vpigqclcqa gyekvedacq acspgffkfe asespclecp |
| 241 | ehtlpspega tsceceegff rapqdpasmp ctrppsaphy ltavgmgakv elrwtppqds |
| 301 | ggredivysv tceqcwpesg ecgpceasvr ysepphgltr tsvtvsdlep hmnytftvea |
| 361 | rngvsglvts rsfrtasvsi nqteppkvrl egrsttslsv swsipppqqs rvwkyevtyr |
| 421 | kkgdsnsynv rrtegfsvtl ddlapdttyl vqvqaltqeg qgagskvhef qtlspegsgn |
| 481 | laviggvavg vvlllvlagv gffihrrrkn qrarqspedv yfskseqlkp lktyvdphty |
| 541 | edpnqavlkf tteihpscvt rqkvigagef gevykgmlkt ssgkkevpva iktlkagyte |
| 601 | kqrvdflgea gimgqfshhn iirlegvisk ykpmmiitey mengaldkfl rekdgefsvl |
| 661 | qlvgmlrgia agmkylanmn yvhrdlaarn ilvnsnlvck vsdfglsrvl eddpeatytt |
| 721 | sggkipirwt apeaisyrkf tsasdvwsfg ivmwevmtyg erpywelsnh evmkaindgf |
| 781 | rlptpmdcps aiyqlmmqcw qqerarrpkf adivsildkl irapdslktl adfdprvsir |
| 841 | lpstsgsegv pfrtvsewle sikmqqyteh fmaagytaie kvvqmtnddi krigvrlpgh |
| 901 | qkriaysllg lkdqvntvgi pi |

Receptor-tyrosine-protein kinase erbB-2, isoform a precursor NP_004439.2 (SEQ
ID NO: 234)

| | |
|---|---|
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dpinnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvqlvtql mpygclldhv renrgrlgsq dllnwcmqia kgmsyledvr |
| 841 | lvhrdlaarn vlvkspnhvk itdfglarll dideteyhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedlgpaspl dstfyrslle dddmgdlvda |
| 1021 | eeylvpqqgf fcpdpapgag gmvhhrhrss strsgggdlt lglepseeea prsplapseg |
| 1081 | agsdvfdgdl gmgaakglqs lpthdpsplq rysedptvpl psetdgyvap ltcspqpeyv |
| 1141 | nqpdvrpqpp spregplpaa rpagatlerp ktlspgkngv vkdvfafgga venpeyltpq |
| 1201 | ggaapqphpp pafspafdnl yywdqdpper gappstfkgt ptaenpeylg ldvpv |

Receptor-tyrosine-protein kinase erbB-2, isoform b NP_001005862.1 (SEQ ID NO:
235)

| | |
|---|---|
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctlvcplhnq evtaedgtqr |
| 301 | cekcskpcar vcyglgmehl revravtsan igefagckki fgslaflpes fdgdpasnta |
| 361 | plqpeqlqvf etleeitgyl yisawpdslp dlsvfqnlqv irgrilhnga ysltlqglgi |
| 421 | swlglrslre lgsglalihh nthlcfvhtv pwdqlfrnph qallhtanrp edecvgegla |
| 481 | chqlcarghc wgpgptqcvn csqflrggec veecrvlqgl preyvnarhc lpchpecqpq |
| 541 | ngsvtcfgpe adqcvacahy kdppfcvarc psgvkpdlsy mpiwkfpdee gacqpcpinc |
| 601 | thscvdlddk gcpaeqrasp ltsiisavvg illvvvlgvv fgilikrrqq kirkytmrrl |
| 661 | lqetelvepl tpsgampnqa qmrilketel rkvkvlgsga fgtvykgiwi pdgenvkipv |
| 721 | aikvlrents pkankeilde ayvmagvgsp yvsrllgicl tstvqlvtql mpygclldhv |
| 781 | renrgrlgsq dllnwcmqia kgmsyledvr lvhrdlaarn vlvkspnhvk itdfglarll |
| 841 | dideteyhad ggkvpikwma lesilrrrft hqsdvwsygv tvwelmtfga kpydgipare |
| 901 | ipdllekger lpqppictid vymimvkcwm idsecrprfr elvsefsrma rdpqrfvviq |
| 961 | nedlgpaspl dstfyrslle dddmgdlvda eeylvpqqgf fcpdpapgag gmvhhrhrss |
| 1021 | strsgggdlt lglepseeea prsplapseg agsdvfdgdl gmgaakglqs lpthdpsplq |
| 1081 | rysedptvpl psetdgyvap ltcspqpeyv nqpdvrpqpp spregplpaa rpagatlerp |
| 1141 | ktlspgkngv vkdvfafgga venpeyltpq ggaapqphpp pafspafdnl yywdqdpper |
| 1201 | gappstfkgt ptaenpeylg ldvpv |

Receptor-tyrosine-protein kinase erbB-2, isoform c NP_001276865.1 (SEQ ID NO:
236)

| | |
|---|---|
| 1 | mprgswkpqv ctgtdmklrl paspethldm lrhlyqgcqv vqgnleltyl ptnaslsflq |
| 61 | diqevqgyvl iahnqvrqvp lqrlrivrgt qlfednyala vldngdpinn ttpvtgaspg |
| 121 | glrelqlrsl teilkggvli qrnpqlcyqd tilwkdifhk nnqlaltlid tnrsrachpc |
| 181 | spmckgsrcw gessedcqsl trtvcaggca rckgplptdc cheqcaagct gpkhsdclac |
| 241 | lhfnhsgice lhcpalvtyn tdtfesmpnp egrytfgasc vtacpynyls tdvgsctlvc |
| 301 | plhnqevtae dgtqrcekcs kpcarvcygl gmehlrevra vtsaniqefa gckkifgsla |
| 361 | flpesfdgdp asntaplqpe qlqvfetlee itgylyisaw pdslpdlsvf qnlqvirgri |
| 421 | lhngaysltl qglgiswlgl rslrelgsgl alihhnthlc fvhtvpwdql frnphqallh |
| 481 | tanrpedecv geglachqlc arghcwgpgp tqcvncsqfl rgqecveecr vlqglpreyv |
| 541 | narhclpchp ecqpqngsvt cfgpeadqcv acahykdppf cvarcpsgvk pdlsympiwk |
| 601 | fpdeegacqp cpincthscv dlddkgcpae qraspltsii savvgillvv vlgvvfgili |
| 661 | krrqqkirky tmrrllqete lvepltpsga mpnqaqmril ketelrkvkv lgsgafgtvy |
| 721 | kgiwipdgen vkipvaikvl rentspkank eildeayvma gvgspyvsrl lgicltstvq |
| 781 | lvtqlmpygc lldhvrenrg rlgsqdllnw cmqiakgmsy ledvrlvhrd laarnvlvks |
| 841 | pnhvkitdfg larlldidet eyhadggkvp ikwmalesil rrrfthqsdv wsygvtvwel |
| 901 | mtfgakpydg ipareipdll ekgerlpqpp ictidvymim vkcwmidsec rprfrelvse |
| 961 | fsrmardpqr fvviqnedlg paspldstfy rslledddmg dlvdaeeylv pqqgffcpdp |
| 1021 | apgaggmvhh rhrssstrsg ggdltlglep seeeaprspl apsegagsdv fdgdlgmgaa |
| 1081 | kglqslpthd psplqrysed ptvplpsetd gyvapltcsp qpeyvnqpdv rpqppspreg |
| 1141 | plpaarpaga tlerpktlsp gkngvvkdvf afggavenpe yltpqggaap qphpppafsp |
| 1201 | afdnlyywdq dppergapps tfkgtptaen peylgldvpv |

Receptor-tyrosine-protein kinase erbB-2, isoform d NP_001276866.1 (SEQ ID NO:
237)

| | |
|---|---|
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dpinnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvqlvtql mpygclldhv renrgrlgsq dllnwcmqia kgmsyledvr |
| 841 | lvhrdlaarn vlvkspnhvk itdfglarll dideteyhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedlgpaspl dstfyrslle dddmgdlvda |
| 1021 | eeylvpqqgf fcpdpapgag gmvhhrhrss strnm |

Receptor-tyrosine-protein kinase erbB-2, isoform e NP_001276867.1 (SEQ ID NO:
238)

| | |
|---|---|
| 1 | mklrlpaspe thldmlrhly qgcqvvqgnl eltylptnas lsflqdiqev qgyvliahnq |
| 61 | vrqvplqrlr ivrgtqlfed nyalavldng dpinnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctlvcplhnq evtaedgtqr |
| 301 | cekcskpcar vcyglgmehl revravtsan iqefagckki fgslaflpes fdgdpasnta |
| 361 | plqpeqlqvf etleeitgyl yisawpdslp dlsvfqnlqv irgrilhnga ysltlqglgi |
| 421 | swlglrslre lgsglalihh nthlcfvhtv pwdqlfrnph qallhtanrp edecvgegla |
| 481 | chqlcarghc wgpgptqcvn csqflrgqec veecrvlqgl preyvnarhc lpchpecqpq |
| 541 | ngsvtcfgpe adqcvacahy kdppfcvarc psgvkpdlsy mpiwkfpdee gacqpcpinc |
| 601 | ths |

Receptor tyrosine-protein kinase erbB-4, isoform JM-a/CVT-1 precursor
NP_005226.1 (SEQ ID NO: 239)

| | |
|---|---|
| 1 | mkpatglwvw vsllvaagtv qpsdsqsvca gtenklssls dleqqyralr kyyencevvm |
| 61 | gnleitsieh nrdlsflrsv revtgyvlva lnqfrylple nlriirgtkl yedryalaif |
| 121 | lnyrkdgnfg lqelglknit eilnggvyvd qnkflcyadt ihwqdivrnp wpsnltlvst |
| 181 | ngssgcgrch ksctgrcwgp tenhcqtltr tvcaeqcdgr cygpyvsdcc hrecaggcsg |
| 241 | pkdtdcfacm nfndsgacvt qcpqtfvynp ttfqlehnfn akytygafcv kkcphnfvvd |
| 301 | ssscvracps skmeveengi kmckpctdic pkacdgigtg slmsaqtvds snidkfinct |
| 361 | kingnliflv tgihgdpyna ieaidpekln vfrtvreitg flniqswppn mtdfsvfsnl |
| 421 | vtiggrvlys glsllilkqq gitslqfqsl keisagniyi tdnsnlcyyh tinwttlfst |
| 481 | inqrivirdn rkaenctaeg mvcnhlcssd gcwgpgpdqc lscrrfsrgr iciescnlyd |
| 541 | gefrefengs icvecdpqce kmedglltch gpgpdnctkc shfkdgpncv ekcpdglqga |
| 601 | nsfifkyadp drechpchpn ctqgcngpts hdqyypwtg hstlpqhart pliaagvigg |
| 661 | lfilvivglt favyvrrksi kkkralrrfl etelvepltp sgtapnqaql rilketelkr |
| 721 | vkvlgsgafg tvykgiwvpe getvkipvai kilnettgpk anvefmdeal imasmdhphl |
| 781 | vrllgvolsp tiqlvtqlmp hgclleyvhe hkdnigsqll lnwcvqiakg mmyleerrlv |
| 841 | hrdlaarnvl vkspnhvkit dfglarlleg dekeynadgg kmpikwmale cihyrkfthq |
| 901 | sdvwsygvti welmtfggkp ydgiptreip dllekgerlp qppictidvy mvmvkcwmid |
| 961 | adsrpkfkel aaefsrmard pqrylviqgd drmklpspnd skffqnllde edledmmdae |
| 1021 | eylvpqafni pppiytsrar idsnrseigh spppaytpms gnqfvyrdgg faaeqgvsvp |
| 1081 | yraptstipe apvaqgatae ifddsccngt lrkpvaphvq edsstqrysa dptvfapers |
| 1141 | prgeldeegy mtpmrdkpkq eylnpveenp fvsrrkngdl qaldnpeyhn asngppkaed |
| 1201 | eyvneplyln tfantlgkae ylknnilsmp ekakkafdnp dywnhslppr stlqhpdylq |
| 1261 | eystkyfykq ngrirpivae npeylsefsl kpgtvlpppp yrhrntvv |

Receptor tyrosine-protein kinase erbB-4, isoform 3M-a/CVT 2 precursor
NP_001036064.1 (SEQ ID NO: 240)

| | |
|---|---|
| 1 | mkpatglwvw vsllvaagtv qpsdsqsvca gtenklssls dleqqyralr kyyencevvm |
| 61 | gnleitsieh nrdlsflrsv revtgyvlva lnqfrylple nlriirgtkl yedryalaif |
| 121 | lnyrkdgnfg lqelglknlt eilnggvyvd qnkflcyadt ihwqdivrnp wpsnltlvst |
| 181 | ngssgcgrch ksctgrcwgp tenhcqtltr tvcaeqcdgr cygpyvsdcc hrecaggcsg |
| 241 | pkdtdcfacm nfndsgacvt qcpqtfvynp ttfqlehnfn akytygafcv kkcphnfvvd |
| 301 | ssscvracps skmeveengi kmckpctdic pkacdgigtg slmsaqtvds snidkfinct |
| 361 | kingnliflv tgihgdpyna ieaidpekln vfrtvreitg flniqswppn mtdfsvfsnl |
| 421 | vtiggrvlys glsllilkqq gitslqfqsl keisagniyi tdnsnlcyyh tinwttlfst |
| 481 | inqrivirdn rkaenctaeg mvcnhlcssd gcwgpgpdqc lscrrfsrgr iciescnlyd |
| 541 | gefrefengs icvecdpqce kmedglltch gpgpdnctkc shfkdgpncv ekcpdglqga |
| 601 | nsfifkyadp drechpchpn ctqgcngpts hdciyypwtg hstlpqhart pliaagvigg |
| 661 | lfilvivglt favyvrrksi kkkralrrfl etelvepltp sgtapnqaql rilketelkr |
| 721 | vkvlgsgafg tvykgiwvpe getvkipvai kilnettgpk anvefmdeal imasmdhphl |
| 781 | vrllgvclsp tiqlvtqlmp hgclleyvhe hkdnigsqll lnwcvqiakg mmyleerrlv |
| 841 | hrdlaarnvl vkspnhvkit dfglarlleg dekeynadgg kmpikwmale cihyrkfthq |
| 901 | sdvwsygvti welmtfggkp ydgiptreip dllekgerlp qppictidvy mvmvkcwmid |
| 961 | adsrpkfkel aaefsrmard pqrylviqgd drmklpspnd skffqnllde edledmmdae |
| 1021 | eylvpqafni pppiytsrar idsnrnqfvy rdggfaaeqg vsvpyrapts tipeapvaqg |
| 1081 | ataeifddsc cngtlrkpva phvqedsstq rysadptvfa persprgeld eegymtpmrd |
| 1141 | kpkqeylnpv eenpfvsrrk ngdlqaldnp eyhnasngpp kaedeyvnep lylntfantl |
| 1201 | gkaeylknni lsmpekakka fdnpdywnhs lpprstlqhp dylqeystky fykqngrirp |
| 1261 | ivaenpeyls efslkpgtvl ppppyrhrnt vv |

Prolyl endopeptidase FAP, isoform 1 NP_004451.2 (SEQ ID NO: 241)

| | |
|---|---|
| 1 | mktwvkivfg vatsavlall vmcivlrpsr vhnseentmr altlkdilng tfsyktffpn |
| 61 | wisgqeylhq sadnnivlyn ietgqsytil snrtmksvna snyglspdrq fvylesdysk |
| 121 | lwrysytaty yiydlsngef vrgnelprpi qylcwspvgs klayvyqnni ylkqrpgdpp |
| 181 | fqitfngren kifngipdwv yeeemlatky alwwspngkf layaefndtd ipviaysyyg |
| 241 | deqyprtini pypkagaknp vvrifiidtt ypayvgpqev pvpamiassd yyfswltwvt |
| 301 | dervclqwlk rvqnvsvlsi cdfredwqtw dcpktqehie esrtgwaggf fvstpvfsyd |
| 361 | aisyykifsd kdgykhihyi kdtvenaiqi tsgkweaini frvtqdslfy ssnefeeypg |
| 421 | rrniyrisig syppskkcvt chlrkercqy ytasfsdyak yyalvcygpg ipistlhdgr |
| 481 | tdqeikilee nkelenalkn iqlpkeeikk levdeitlwy kmilppqfdr skkyplliqv |
| 541 | yggpcsqsvr svfavnwisy laskegmvia lvdgrgtafq gdkllyavyr klgvyevedq |
| 601 | itavrkfiem gfidekriai wgwsyggyvs slalasgtgl fkcgiavapv ssweyyasvy |
| 661 | terfmglptk ddnlehykns tvmaraeyfr nvdyllihgt addnvhfqns aqiakalvna |
| 721 | qvdfqamwys dqnhglsgls tnhlythmth flkqcfslsd |

Prolyl endopeptidase FAP, isoform 2 NP_001278736.1 (SEQ ID NO: 242)

| | |
|---|---|
| 1 | mktwvkivfg vatsavlall vmcivlrpsr vhnseentmr altlkdilng tfsyktffpn |
| 61 | wisgqeylhq sadnnivlyn ietgqsytil snrtmlwrys ytatyyiydl sngefvrgne |
| 121 | lprpiqylcw spvgsklayv yqnniylkqr pgdppfqitf ngrenkifng ipdwvyeeem |
| 181 | latkyalwws pngkflayae fndtdipvia ysyygdeqyp rtinipypka gaknpvvrif |
| 241 | iidttypayv gpqevpvpam iassdyyfsw ltwvtdervc lqwlkrvqnv svlsicdfre |
| 301 | dwqtwdcpkt qehieesrtg waggffvstp vfsydaisyy kifsdkdgyk hihyikdtve |
| 361 | naiqitsgkw eainifrvtq dslfyssnef eeypgrrniy risigsypps kkcvtchlrk |
| 421 | ercqyytasf sdyakyyalv cygpgipist lhdgrtdqei kileenkele nalkniqlpk |
| 481 | eeikklevde itlwykmilp pqfdrskkyp lliqvyggpc sgsvrsvfav nwisylaske |
| 541 | gmvialvdgr gtafqgdkll yavyrklgvy evedqitavr kfiemgfide kriaiwgwsy |
| 601 | ggyvsslala sgtglfkcgi avapvsswey yasvyterfm glptkddnle hyknstvmar |
| 661 | aeyfrnvdyl lihgtaddnv hfqnsaqiak alvnaqvdfq amwysdqnhg lsglstnhly |
| 721 | thmthflkqc fslsd |

Glutamate carboxypeptidase 2, isoform 1 NP_004467.1 (SEQ ID NO: 243)

| | |
|---|---|
| 1 | mwnllhetds avatarrprw lcagalvlag gffllgflfg wfikssneat nitpkhnmka |
| 61 | fldelkaeni kkflynftqi phlagteqnf qlakqiqsqw kefgldsvel ahydvllsyp |
| 121 | nkthpnyisi inedgneifn tslfeppppg yenvsdivpp fsafspqgmp egdlvyvnya |
| 181 | rtedffkler dmkincsgki viarygkvfr gnkvknaqla gakgvilysd padyfapgvk |
| 241 | sypdgwnlpg ggvqrgniln lngagdpltp gypaneyayr rgiaeavglp sipvhpigyy |
| 301 | daqkllekmg gsappdsswr gslkvpynvg pgftgnfstq kvkmhihstn evtriynvig |
| 361 | tlrgavepdr yvilgghrds wvfggidpqs gaavvheivr sfgtlkkegw rprrtilfas |
| 421 | wdaeefgllg stewaeensr llqergvayi nadssiegny tlrvdctplm yslvhnltke |
| 481 | lkspdegfeg kslyeswtkk spspefsgmp risklgsgnd fevffqrlgi asgrarytkn |
| 541 | wetnkfsgyp lyhsvyetye lvekfydpmf kyhltvaqvr ggmvfelans ivlpfdcrdy |
| 601 | avvlrkyadk iysismkhpq emktysysfd slfsavknft eiaskfserl qdfdksnpiv |
| 661 | lrmmndqlmf lerafidplg lpdrpfyrhv iyapsshnky agesfpgiyd alfdieskvd |
| 721 | pskawgevkr qiyvaaftvq aaaetlseva |

Glutamate carboxypeptidase 2, isoform 2 NP_001014986.1 (SEQ ID NO: 244)

| | |
|---|---|
| 1 | mwnllhetds avatarrprw lcagalvlag gffllgflfg wfikssneat nitpkhnmka |
| 61 | fldelkaeni kkflynftqi phlagteqnf qlakqiqsqw kefgldsvel ahydvllsyp |
| 121 | nkthpnyisi inedgneifn tslfeppppg yenvsdivpp fsafspqgmp egdlvyvnya |
| 181 | rtedffkler dmkincsgki viarygkvfr gnkvknaqla gakgvilysd padyfapgvk |
| 241 | sypdgwnlpg ggvqrgniln lngagdpltp gypaneyayr rgiaeavglp sipvhpigyy |
| 301 | daqkllekmg gsappdsswr gslkvpynvg pgftgnfstq kvkmhihstn evtriynvig |
| 361 | tlrgavepdr yvilgghrds wvfggidpqs gaavvheivr sfgtlkkegw rprrtilfas |
| 421 | wdaeefgllg stewaeensr llgergvayi nadssiegny tlrvdctplm yslvhnitke |
| 481 | lkspdegfeg kslyeswtkk spspefsgmp risklgsgnd fevffqrlgi asgrarytkn |
| 541 | wetnkfsgyp lyhsvyetye lvekfydpmf kyhltvaqvr ggmvfelans ivlpfdcrdy |
| 601 | avvlrkyadk iysismkhpq emktysysfd slfsavknft eiaskfserl qdfdkskhvi |
| 661 | yapsshnkya gesfpgiyda lfdieskvdp skawgevkrq iyvaaftvqa aaetlseva |

Glutamate carboxypeptidase 2, isoform 3 NP_001180400.1 (SEQ ID NO: 245)

| | |
|---|---|
| 1 | mtagssyplf laayactgcl aerlgwfiks sneatnitpk hnmkafldel kaenikkfly |
| 61 | nftqiphlag teqnfqlakq iqsqwkefgl dsvelahydv llsypnkthp nyisiinedg |
| 121 | neifntslfe ppppgyenvs divppfsafs pqgmpegdlv yvnyartedf fklerdmkin |
| 181 | csgkiviary gkvfrgnkvk naqlagakgv ilysdpadyf apgvksypdg wnlpgggvqr |
| 241 | gnilnlngag dpltpgypan eyayrrgiae avglpsipvh pigyydaqkl lekmggsapp |
| 301 | dsswrgslkv pynvgpgftg nfstqkvkmh ihstnevtri ynvigtlrga vepdryvilg |
| 361 | ghrdswvfgg idpqsgaavv heivrsfgtl kkegwrprrt ilfaswdaee fgllgstewa |
| 421 | eensrllqer gvayinadss iegnytlrvd ctplmyslvh nitkelkspd egfegkslye |
| 481 | swtkkspspe fsgmpriskl gsgndfevff qrlgiasgra rytknwetnk fsgyplyhsv |
| 541 | yetyelvekf ydpmfkyhlt vaqvrggmvf elansivlpf dcrdyavvlr kyadkiysis |
| 601 | mkhpqemkty sysfdslfsa vknfteiask fserlqdfdk snpivlrmmn dqlmfleraf |
| 661 | idplglpdrp fyrhviyaps shnkyagesf pgiydalfdi eskvdpskaw gevkrqiyva |
| 721 | aftvqaaaet lseva |

Glutamate carboxypeptidase 2, isoform 4 NP_001180401.1 (SEQ ID NO: 246)

| | |
|---|---|
| 1 | mtagssyplf laayactgcl aerlgwfiks sneatnitpk hnmkafldel kaenikkfly |
| 61 | nftqiphlag teqnfqlakq iqsqwkefgl dsvelahydv llsypnkthp nyisiinedg |
| 121 | neifntslfe ppppgyenvs divppfsafs pqgmpegdlv yvnyartedf fklerdmkin |
| 181 | csgkiviary gkvfrgnkvk naqlagakgv ilysdpadyf apgvksypdg wnlpgggvqr |
| 241 | gnilnlngag dpltpgypan eyayrrgiae avglpsipvh pigyydaqkl lekmggsapp |
| 301 | dsswrgslkv pynvgpgftg nfstqkvkmh ihstnevtri ynvigtlrga vepdryvilg |
| 361 | ghrdswvfgg idpqsgaavv heivrsfgtl kkegwrprrt ilfaswdaee fgllgstewa |
| 421 | eensrllqer gvayinadss iegnytlrvd ctplmyslvh nitkelkspd egfegkslye |
| 481 | swtkkspspe fsgmpriskl gsgndfevff qrlgiasgra rytknwetnk fsgyplyhsv |
| 541 | yetyelvekf ydpmfkyhlt vaqvrggmvf elansivlpf dcrdyavvlr kyadkiysis |
| 601 | mkhpqemkty sysfdslfsa vknfteiask fserlqdfdk skhviyapss hnkyagesfp |
| 661 | giydalfdie skvdpskawg evkrqiyvaa ftvqaaaetl seva |

Glutamate carboxypeptidase 2, isoform 5 NP_001180402.1 (SEQ ID NO: 247)

| | |
|---|---|
| 1 | mggsappdss wrgslkvpyn vgpgftgnfs tqkvkmhihs tnevtriynv igtlrgavep |
| 61 | dryvilgghr dswvfggidp qsgaavvhei vrsfgtlkke gwrprrtilf aswdaeefgl |
| 121 | lgstewaeen srllqergva yinadssieg nytlrvdctp lmyslvhnit kelkspdegf |
| 181 | egkslyeswt kkspspefsg mprisklgsg ndfevffqrl giasgraryt knwetnkfsg |
| 241 | yplyhsvyet yelvekfydp mfkyhltvaq vrggmvfela nsivlpfdcr dyavvlrkya |
| 301 | dkiysismkh pqemktysvs fdslfsavkn fteiaskfse rlqdfdksnp ivlrmmndql |
| 361 | mflerafidp lglpdrpfyr hviyapsshn kyagesfpgi ydalfdiesk vdpskawgev |
| 421 | krqiyvaaft vqaaaetlse va |

Glutamate carboxypeptidase 2, isoform 6 NP_001338165.1 (SEQ ID NO: 248)

| | |
|---|---|
| 1 | mkafldelka enikkflynf tqiphlagte qnfqlakqiq sqwkefglds velahydvll |
| 61 | sypnkthpny isiinedgne ifntslfepp ppgyenvsdi vppfsafspq gmpegdlvyv |
| 121 | nyartedffk lerdmkincs gkiviarygk vfrgnkvkna qlagakgvil ysdpadyfap |
| 181 | gvksypdgwn lpgggvqrgn ilnlngagdp ltpgypaney ayrrgiaeav glpsipvhpi |
| 241 | gyydaqklle kmggsappds swrgslkvpy nvgpgftgnf stqkvkmhih stnevtriyn |
| 301 | vigtlrgave pdryvilggh rdswvfggid pqsgaavvhe ivrsfgtlkk egwrprrtil |
| 361 | faswdaeefg llgstewaee nsrllqergv ayinadssie gnytlrvdct plmyslvhnl |
| 421 | tkelkspdeg fegkslyesw tkkspspefs gmprisklgs gndfevffqr lgiasgrary |
| 481 | tknwetnkfs gyplyhsvye tyelvekfyd pmfkyhltva qvrggmvfel ansivlpfdc |
| 541 | rdyavvlrky adkiysismk hpqemktysv sfdslfsavk nfteiaskfs erlqdfdksk |
| 601 | hviyapsshn kyagesfpgi ydalfdiesk vdpskawgev krqiyvaaft vqaaaetlse |
| 661 | va |

Fos-related antigen 1, isoform 1 NP_005429.1 (SEQ ID NO: 249)

| | |
|---|---|
| 1 | mfrdfgepgp ssgngggygg paqppaaaqa aqqkfhlvps intmsgsqel qwmvqphflg |
| 61 | pssyprplty pqysppqprp gviralgppp gvrrrpceqi speeeerrrv rrernklaaa |
| 121 | kcrnrrkelt dflqaetdkl edeksglqre ieelqkqker lelvleahrp ickipegake |
| 181 | gdtgstsgts sppaperpvp cislspgpvl epealhtptl mttpsltpft pslvftypst |
| 241 | pepcasahrk sssssgdpss dplgsptlla l |

Fos-related antigen 1, isoform 2 NP_001287773.1 (SEQ ID NO: 250)

| | |
|---|---|
| 1 | mfrdfgepgp ssgngggygg paqppaaaqa aqqkfhlvps intmsgsqel qwmvqphflg |
| 61 | pssyprplty pqysppqprp gviralgppp gvrrrpceqe tdkledeksg lqreieelqk |
| 121 | qkerlelvle ahrpickipe gakegdtgst sgtssppapc rpvpcislsp gpvlepealh |
| 181 | tptlmttpsl tpftpslvft ypstpepcas ahrksssssg dpssdplgsp tllal |

Fos-related antigen 1, isoform 3 NP_001287784.1 (SEQ ID NO: 251)

| | |
|---|---|
| 1 | mfrdfgepgp ssgngggygg paqppaaaqa aqqkfhlvps intmsgsqel qwmvqphflg |
| 61 | pssyprplty pqysppqprp gviralgppp gvrrrpceqp ggrgappska raeqagcgqv |
| 121 | qepeegtdrl paggd |

Fos-related antigen 1, isoform 4 NP_001287785.1 (SEQ ID NO: 252)

| | |
|---|---|
| 1 | mfrdfgepgp ssgngggygg paqppaaaqa aqqispeeee rrrvrrernk laaakcrnrr |
| 61 | keltdflqae tdkledeksg lqreieelqk qkerlelvle ahrpickipe gakegdtgst |
| 121 | sgtssppapc rpvpcislsp gpvlepealh tptlmttpsl tpftpslvft ypstpepcas |
| 181 | ahrksssssg dpssdplgsp tllal |

Fos-related antigen 1, isoform 5 NP_001287786.1 (SEQ ID NO: 253)

| | |
|---|---|
| 1 | mfrdfgepgp ssgngggygg paqppaaaqa aqqetdkled eksglqreie elqkqkerle |
| 61 | lvleahrpic kipegakegd tgstsgtssp paperpvpci slspgpvlep ealhtptlmt |
| 121 | tpsltpftps lvftypstpe pcasahrkss sssgdpssdp lgsptllal |

G antigen 1 NP_001035753.1 (SEQ ID NO: 254)

| | |
|---|---|
| 1 | mswrgrstyy wprprryvqp pemigpmrpe qfsdevepat peegepatqr qdpaaaqege |
| 61 | degasagqgp kpeadsqeqg hpqtgceced gpdgqemdpp npeevktpee gegqsqc |

G antigen 121 NP_001465.1 (SEQ ID NO: 255)

| | |
|---|---|
| 1 | mswrgrstyy wprprryvqp pemigpmrpe qfsdevepat peegepatqr qdpaaaqege |
| 61 | degasagqgp kpeadsqeqg hpqtgceced gpdgqemdpp npeevktpee gekqsqc |

Galectin-1 NP_002296.1 (SEQ ID NO: 256)

| | |
|---|---|
| 1 | macglvasnl nlkpgeclrv rgevapdaks fvlnlgkdsn nlclhfnprf nahgdantiv |
| 61 | cnskdggawg teqreavfpf qpgsvaevci tfdqanltvk lpdgyefkfp nrinleainy |
| 121 | maadgdfkik cvafd |

Galectin-3 isoform 1 NP_002297.2 (SEQ ID NO: 257)

| | |
|---|---|
| 1 | madnfslhda lsgsgnpnpq gwpgawgnqp agaggypgas ypgaypgqap pgaypgqapp |
| 61 | gaypgapgay pgapapgvyp gppsgpgayp ssgqpsatga ypatgpygap agplivpynl |
| 121 | plpggvvprm litilgtvkp nanrialdfq rgndvafhfn prfnennrry ivcntkldnn |
| 181 | wgreerqsvf pfesgkpfki qvlvepdhfk vavndahllq ynhrvkklne isklgisgdi |
| 241 | dltsasytmi |

Galectin-3, isoform 3 NP_001344607.1 (SEQ ID NO: 258)

| | |
|---|---|
| 1 | mhsktpcgcf kpwkmadnfs lhdalsgsgn pnpqgwpgaw gnqpagaggy pgasypgayp |
| 61 | gqappgaypg qappgaypga pgaypgapap gvypgppsgp gaypssgqps atgaypatgp |
| 121 | ygapagpliv pynlplpggv vprmlitilg tvkpnanria ldfqrgndva fhfnprfnen |
| 181 | nrrvivcntk ldnnwgreer qsvfpfesgk pfkiqvlvep dhfkvavnda hllqynhrvk |
| 241 | klneisklgi sgdidltsas ytmi |

Galectin-9 short NP_002299.2 (SEQ ID NO: 259)

| | |
|---|---|
| 1 | mafsgsqapy lspavpfsgt iqgglqdglq itvngtvlss sgtrfavnfq tgfsgndiaf |
| 61 | hfnprfedgg yvvcntrqng swgpeerkth mpfqkgmpfd lcflvqssdf kvmvngilfv |
| 121 | qyfhrvpfhr vdtisvngsv qlsyisfqpp gvwpanpapi tqtvihtvqs apgqmfstpa |
| 181 | ippmmyphpa ypmpfittil gglypsksil lsgtvlpsaq rfhinlcsgn hiafhlnprf |
| 241 | denavvrntq idnswgseer slprkmpfvr gqsfsvwilc eahclkvavd gqhlfeyyhr |
| 301 | lrnlptinrl evggdiqlth vqt |

Galectin-9 long NP_033665.1 (SEQ ID NO: 260)

| | |
|---|---|
| 1 | mafsgsqapy lspavpfsgt iqgglqdglq itvngtvlss sgtrfavnfq tgfsgndiaf |
| 61 | hfnprfedgg yvvcntrqng swgpeerkth mpfqkgmpfd lcflvqssdf kvmvngilfv |
| 121 | qyfhrvpfhr vdtisvngsv qlsyisfqnp rtvpvqpafs tvpfsqpvcf pprprgrrqk |
| 181 | ppgvwpanpa pitqtvihtv qsapgqmfst paippmmyph paypmpfitt ilgglypsks |
| 241 | illsgtvlps aqrfhinlcs gnhiafhlnp rfdenavvrn tqidnswgse erslprkmpf |
| 301 | vrgqsfsvwi lceahclkva vdgqhlfeyy hrlrnlptin rlevggdiql thvqt |

Galectin-9 isoform 3 NP_001317092.1 (SEQ ID NO: 261)

| | |
|---|---|
| 1 | mafsgsqapy lspavpfsgt iqgglqdglq itvngtvlss sgtrfavnfq tgfsgndiaf |
| 61 | hfnprfedgg yvvcntrqng swgpeerkth mpfqkgmpfd lcflvqssdf kvmvngilfv |
| 121 | qyfhrvpfhr vdtisvngsv qlsyisfqpp gvwpanpapi tqtvihtvqs apgqmfstpa |
| 181 | ippmmyphpa ypmpfittil gglypsksil lsgtvlpsaq rcgscvklta srwpwmvstc |
| 241 | lnttia |

Premelanosome protein, isoform 1 preprotein NP_001186983.1 (SEQ ID NO: 262)

| | |
|---|---|
| 1 | mdlvlkrcll hlavigalla vgatkvprnq dwlgvsrqlr tkawnrqlyp ewteaqrldc |
| 61 | wrggqvslkv sndgptliga nasfsialnf pgsqkvlpdg qviwvnntii ngsqvwggqp |
| 121 | vypqetddac ifpdggpcps gswsqkrsfv yvwktwgqyw qvlggpvsgl sigtgramlg |
| 181 | thtmevtvyh rrgsrsyvpl ahsssaftit dqvpfsysys qlraldggnk hflrnqpltf |
| 241 | alqlhdpsgy laeadlsytw dfgdssgtli sralvvthty lepgpvtaqv vlqaaiplts |
| 301 | cgsspvpgtt dghrptaeap nttagqvptt evvgttpgqa ptaepsgtts vqvpttevis |
| 361 | tapvqmptae stgmtpekvp vsevmgttla emstpeatgm tpaevsivvl sgttaaqvtt |
| 421 | tewvettare lpipepegpd assimstesi tgslgplldg tatlrlvkrq vpldcvlyry |
| 481 | gsfsvtldiv qgiesaeilq avpsgegdaf eltvscqggl pkeacmeiss pgcqppaqrl |
| 541 | cqpvlpspac qlvlhqilkg gsgtyclnvs ladtnslavv stqlimpvpg illtgqeagl |
| 601 | gqvplivgil lvlmavvlas liyrrrlmkg dfsvpqlphs sshwlrlpri fcscpigens |
| 661 | pllsgqqv |

Premelanosome protein, isoform 2 precursor NP_001186982.1 (SEQ ID NO: 263)

| | |
|---|---|
| 1 | mdlvlkrcll hlavigalla vgatkgsqvw ggqpvypqet ddacifpdgg pcpsgswsqk |
| 61 | rsfvyvwktw gqywqvlggp vsglsigtgr amlgthtmev tvyhrrgsrs yvplahsssa |
| 121 | ftitdqvpfs vsysqlrald ggnkhflrnq pltfalqlhd psgylaeadl sytwdfgdss |
| 181 | gtlisralvv thtylepgpv taqvvlqaai pltscgsspv pgttdghrpt aeapnttagq |
| 241 | vpttevvgtt pgqaptaeps gttsvqvptt evistapvqm ptaestgmtp ekvpvsevmg |
| 301 | ttlaemstpe atgmtpaevs ivvlsgttaa qvtttewvet tarelpipep egpdassims |
| 361 | tesitgslgp lldgtatlrl vkrqvpldcv lyrygsfsvt ldivqgiesa eilqavpsge |
| 421 | gdafeltvsc qgglpkeacm eisspgcqpp aqrlcqpvlp spacqlvlhq ilkggsgtyc |
| 481 | lnvsladtns lavvstqlim pgqeaglgqv plivgillvl mavvlasliy rrrlmkqdfs |
| 541 | vpqlphsssh wlrlprifcs cpigenspll sgqqv |

Premelanosome protein, isoform 3 preprotein NP_008859.1 (SEQ ID NO: 264)

| | |
|---|---|
| 1 | mdlvlkrcll hlavigalla vgatkvprnq dwlgvsrqlr tkawnrqlyp ewteaqrldc |
| 61 | wrggqvslkv sndgptliga nasfsialnf pgsqkvlpdg qviwvnntii ngsqvwggqp |
| 121 | vypqetddac ifpdggpcps gswsqkrsfv yvwktwgqyw qvlggpvsgl sigtgramlg |
| 181 | thtmevtvyh rrgsrsyvpl ahsssaftit dqvpfsysvs qlraldggnk hflrnqpltf |
| 241 | alqlhdpsgy laeadlsytw dfgdssgtli sralvvthty lepgpvtaqv vlqaaiplts |
| 301 | cgsspvpgtt dghrptaeap nttagqvptt evvgttpgqa ptaepsgtts vqvpttevis |
| 361 | tapvqmptae stgmtpekvp vsevmgttla emstpeatgm tpaevsivvl sgttaaqvtt |
| 421 | tewvettare lpipepegpd assimstesi tgslgplldg tatlrlvkrq vpldcvlyry |
| 481 | gsfsvtldiv qgiesaeilq avpsgegdaf eltvscqggl pkeacmeiss pgcqppaqrl |
| 541 | cqpvlpspac qlvlhqilkg gsgtyclnvs ladtnslavv stqlimpgqe aglgqvpliv |
| 601 | gillvlmavv lasliyrrrl mkgdfsvpql phssshwlrl prifcscpig enspllsgqq |
| 661 | v |

Premelanosome protein, isoform 4 preprotein NP_001307050.1 (SEQ ID NO: 265)

| | |
|---|---|
| 1 | mdlvlkrcll hlavigalla vgatkvprnq dwlgvsrqlr tkawnrqlyp ewteaqrldc |
| 61 | wrggqvslkv sndgptliga nasfsialnf pgsqkvlpdg qviwvnntii ngsqvwggqp |
| 121 | vypqetddac ifpdggpcps gswsqkrsfv yvwktwgqyw qvlggpvsgl sigtgramlg |
| 181 | thtmevtvyh rrgsrsyvpl ahsssaftit dqvpfsvsvs qlraldggnk hflrnqpltf |
| 241 | alqlhdpsgy laeadlsytw dfgdssgtli sralvvthty lepgpvtaqv vlqaaiplts |
| 301 | cgsspvpgtt dghrptaeap nttagqvptt evvgttpgqa ptaepsgtts vqvpttevis |
| 361 | tapvqmptae staaqvttte wvettarelp ipepegpdas simstesitg slgplldgta |
| 421 | tlrlvkrqvp ldcvlyrygs fsvtldivqg iesaeilqav psgegdafel tvscqgglpk |
| 481 | eacmeisspg cqppaqrlcq pvlpspacql vlhqilkggs gtyclnvsla dtnslavvst |
| 541 | qlimpvpgil ltgqeaglgq vplivgillv lmavvlasli yrrrlmkqdf svpqlphsss |
| 601 | hwlrlprifc scpigenspl lsgqqv |

Premelanosome protein, isoform 5 preprotein NP_001307051.1 (SEQ ID NO: 266)

| | |
|---|---|
| 1 | mdlvlkrcll hlavigalla vgatkvprnq dwlgvsrqlr tkawnrqlyp ewteaqrldc |
| 61 | wrggqvslkv sndgptliga nasfsialnf pgsqkvlpdg qviwvnntii ngsqvwggqp |
| 121 | vypqetddac ifpdggpcps gswsqkrsfv yvwktwgqyw qvlggpvsgl sigtgramlg |
| 181 | thtmevtvyh rrgsrsyvpl ahsssaftit dqvpfsysys qlraldggnk hflrnqpltf |
| 241 | alqlhdpsgy laeadlsytw dfgdssgtli sralvvthty lepgpvtaqv vlqaaiplts |
| 301 | cgsspvpgtt dghrptaeap nttagqvptt evvgttpgqa ptaepsgtts vqvpttevis |
| 361 | tapvqmptae staaqvttte wvettarelp ipepegpdas simstesitg slgplldgta |
| 421 | tlrlvkrqvp ldcvlyrygs fsvtldivqg iesaeilqav psgegdafel tvscqgglpk |
| 481 | eacmeisspg cqppaqrlcq pvlpspacql vlhqilkggs gtyclnvsla dtnslavvst |
| 541 | qlimpgqeag lgqvplivgi llvlmavvla sliyrrrlmk qdfsvpqlph ssshwlrlpr |
| 601 | ifcscpigen spllsgqqv |

Glutamate receptor ionotropic, NMDA 2A, isoform 1 precursor NP_000824.1,
NP_001127879.1 (SEQ ID NO: 267)

| | |
|---|---|
| 1 | mgrvgywtll vlpallvwrg papsaaaekg ppalniavml ghshdvtere lrtlwgpeqa |
| 61 | aglpldvnvv allmnrtdpk slithvcdlm sgarihglvf gddtdqeava qmldfissht |
| 121 | fvpilgihgg asmimadkdp tstffqfgas iqqqatvmlk imqdydwhvf slvttifpgy |
| 181 | refisfvktt vdnsfvgwdm qnvitldtsf edaktqvglk kihssvilly cskdeavlil |
| 241 | searslgltg ydffwivpsl vsgntelipk efpsglisvs yddwdyslea rvrdgigilt |
| 301 | taassmlekf syipeakasc ygqmerpevp mhtlhpfmvn vtwdgkdlsf teegyqvhpr |
| 361 | lvvivlnkdr ewekvgkwen htlslrhavw pryksfsdce pddnhlsivt leeapfvive |
| 421 | didpltetcv rntvperkfv kinnstnegm nvkkcckgfc idilkklsrt vkftydlylv |
| 481 | tngkhgkkvn nvwngmigev vyqravmavg sltineerse vvdfsvpfve tgisvmvsrs |
| 541 | ngtvspsafl epfsasvwvm mfvmllivsa iavfvfeyfs pvgynrnlak gkaphgpsft |
| 601 | igkaiwllwg lvfnnsvpvq npkgttskim vsvwaffavi flasytanla afmiqeefvd |
| 661 | qvtglsdkkf qrphdysppf rfgtvpngst ernirnnypy mhqymtkfnq kgvedalvsl |
| 721 | ktgkldafiy daavlnykag rdegcklvti gsgyifattg ygialqkgsp wkrqidlall |
| 781 | qfvgdgemee letlwltgic hneknevmss qldidnmagv fymlaaamal slitfiwehl |
| 841 | fywklrfcft gvcsdrpgll fsisrgiysc ihgvhieekk kspdfnitgs qsnmlkllrs |
| 901 | aknissmsnm nssrmdspkr aadfiqrgsl imdmvsdkgn lmysdnrsfq gkesifgdnm |
| 961 | nelqtfvanr qkdnlnnyvf qgqhpltlne snpntvevav steskansrp rqlwkksvds |
| 1021 | irqdslsqnp vsqrdeatae nrthslkspr ylpeemahsd isetsnratc hrepdnsknh |
| 1081 | ktkdnfkrsv askypkdcse vertylktks ssprdkiyti dgekepgfhl dppqfvenvt |
| 1141 | lpenvdfpdp yqdpsenfrk gdstlpmnrn plhneeglsn ndqyklyskh ftlkdkgsph |
| 1201 | setseryrqn sthcrsclsn mptysghftm rspfkcdacl rmgnlydide dgmlqetgnp |
| 1261 | atgeqvyqqd waqnnalqlq knklrisrqh sydnivdkpr eldlsrpsrs islkdrerll |
| 1321 | egnfygslfs vpssklsgkk sslfpqgled skrsksllpd htsdnpflhs hrddqrlvig |
| 1381 | rcpsdpykhs lpsqavndsy lrsslrstas ycsrdsrghn dvyisehvmp yaanknnmys |
| 1441 | tprvlnscsn rrvykkmpsi esdv |

Glutamate receptor ionotropic, NMDA 2A, isoform 2 precursor NP_001127880.1
(SEQ ID NO: 268)

| | |
|---|---|
| 1 | mgrvgywtll vlpallvwrg papsaaaekg ppalniavml ghshdvtere lrtlwgpeqa |
| 61 | aglpldvnvv allmnrtdpk slithvcdlm sgarihglvf gddtdqeava qmldfissht |
| 121 | fvpilgihgg asmimadkdp tstffqfgas iqqqatvmlk imgdydwhvf slvttifpgy |
| 181 | refisfvktt vdnsfvgwdm qnvitldtsf edaktqvqlk kihssvilly cskdeavlil |
| 241 | searslgltg ydffwivpsl vsgntelipk efpsglisvs yddwdyslea rvrdgigilt |
| 301 | taassmlekf syipeakasc ygqmerpevp mhtlhpfmvn vtwdgkdlsf teegyqvhpr |
| 361 | lvvivlnkdr ewekvgkwen htlslrhavw pryksfsdce pddnhlsivt leeapfvive |
| 421 | didpltetcv rntvpcrkfv kinnstnegm nvkkcckgfc idilkklsrt vkftydlylv |
| 481 | tngkhgkkvn nvwngmigev vyqravmavg sltineerse vvdfsvpfve tgisvmvsrs |
| 541 | ngtvspsafl epfsasvwvm mfvmllivsa iavfvfeyfs pvgynrnlak gkaphgpsft |
| 601 | igkaiwllwg lvfnnsvpvq npkgttskim vsvwaffavi flasytanla afmiqeefvd |
| 661 | qvtglsdkkf qrphdysppf rfgtvpngst ernirnnypy mhqymtkfnq kgvedalvsl |
| 721 | ktgkldafiy daavinykag rdegcklvti gsgyifattg ygialqkgsp wkrqidlall |
| 781 | qfvgdgemee letlwltgic hneknevmss qldidnmagv fymlaaamal slitfiwehl |
| 841 | fywklrfcft gvcsdrpgll fsisrgiysc ihgvhieekk kspdfnltgs qsnmlkllrs |
| 901 | aknissmsnm nssrmdspkr aadfiqrgsl imdmvsdkgn lmysdnrsfq gkesifgdnm |
| 961 | nelqtfvanr qkdnlnnyvf qgqhpltlne snpntvevav steskansrp rqlwkksvds |
| 1021 | irqdslsqnp vsqrdeatae nrthslkspr ylpeemahsd isetsnratc hrepdnsknh |
| 1081 | ktkdnfkrsv askypkdcse vertylktks ssprdkiyti dgekepgfhl dppqfvenvt |
| 1141 | lpenvdfpdp yqdpsenfrk gdstlpmnrn plhneeglsn ndqyklyskh ftlkdkgsph |
| 1201 | setseryrqn sthcrsclsn mptysghftm rspfkcdacl rmgnlydide dqmlqetgmt |
| 1261 | nawllgdapr tltntrchpr r |

Metabotropic glutamate receptor 3 precursor NP_000831.2 (SEQ ID NO: 269)

| | |
|---|---|
| 1 | mkmltrlqvl tlalfskgfl lslgdhnflr reikiegdlv lgglfpinek gtgteecgri |
| 61 | nedrgiqrle amlfaidein kddyllpgvk lgvhildtcs rdtyaleqsl efvrasltkv |
| 121 | deaeymcpdg syaiqenipl liagviggsy ssysiqvanl lrlfqipqis yastsaklsd |
| 181 | ksrydyfart vppdfyqaka maeilrffnw tyvstvaseg dygetgieaf eqearlrnic |
| 241 | iataekvgrs nirksydsvi rellqkpnar vvvlfmrsdd sreliaaasr anasftwvas |
| 301 | dgwgaqesii kgsehvayga itlelasqpv rqfdryfqsl npynnhrnpw frdfweqkfq |
| 361 | cslqnkrnhr rvcdkhlaid ssnyeqeski mfvvnavyam ahalhkmqrt lcpnttklcd |
| 421 | amkildgkkl ykdyllkinf tapfnpnkda dsivkfdtfg dgmgrynvfn fqnvggkysy |
| 481 | lkvghwaetl sldvnsihws rnsvptsqcs dpcapnemkn mqpgdvccwi cipcepyeyl |
| 541 | adeftcmdcg sgqwptadlt gcydlpedyi rwedawaigp vtiaclgfmc tcmvvtvfik |
| 601 | hnntplvkas grelcyillf gvglsycmtf ffiakpspvi calrrlglgs sfaicysall |
| 661 | tktnciarif dgvkngaqrp kfispssqvf iclglilvqi vmvsvwlile apgtrrytla |
| 721 | ekretvilkc nvkdssmlis ltydvilvil ctvyafktrk cpenfneakf igftmyttci |
| 781 | iwlaflpify vtssdyrvqt ttmcisvsls gfvvlgclfa pkvhiilfqp qknvvthrlh |
| 841 | lnrfsysgtg ttysqssast yvptvcngre vldsttssl |

HPV E6 concoprotein, NP_041325.1 (SEQ ID NO: 270)

| | |
|---|---|
| 1 | mhqkrtamfq dpqerprklp qlctelqtti hdiilecvyc kqqllrrevy dfafrdlciv |
| 61 | yrdgnpyavc dkclkfyski seyrhycysl ygttleqqyn kplcdllirc incqkplcpe |
| 121 | ekqrhldkkq rfhnirgrwt grcmsccrss rtrretql |

HPV E7 Oncoprotein, NP_041326.1 (SEQ ID NO: 271)

| | |
|---|---|
| 1 | mhgdtptlhe ymldlqpett dlycyeqlnd sseeedeidg pagqaepdra hynivtfcck |
| 61 | cdstlrlcvq sthvdirtle dllmgtlgiv cpicsqkp |

GTPase HRas, isoform 1 NP_001123914.1, NP_005334.1 (SEQ ID NO: 272)

| | |
|---|---|
| 1 | mteyklvvvg aggvgksalt iqliqnhfvd eydptiedsy rkqvvidget clldildtag |
| 61 | qeeysamrdq ymrtgegflc vfainntksf edihqyreqi krvkdsddvp mvlvgnkcdl |
| 121 | aartvesrqa qdlarsygip yietsaktrq gvedafytlv reirqhklrk lnppdesgpg |
| 181 | cmsckcvls |

GTPase HRas, isoform 3 NP_001304983.1 (SEQ ID NO: 273)

| | |
|---|---|
| 1 | mtcpwcwwgt svtwlhalwn lgrlrtspea tasptsrprp rpgraaalal apapgpsgtp |
| 61 | rdpcdpaapr agvedafytl vreirqhklr klnppdesgp gcmsckcvls |

GTPase HRas, isoform 2 NP_789765.1 (SEQ ID NO: 274)

| | |
|---|---|
| 1 | mteyklvvvg aggvgksalt iqliqnhfvd eydptiedsy rkqvvidget clldildtag |
| 61 | qeeysamrdq ymrtgegflc vfainntksf edihqyreqi krvkdsddvp mvlvgnkcdl |
| 121 | aartvesrqa qdlarsygip yietsaktrq gsrsgsssss gtlwdppgpm |

Vascular endothelial growth factor receptor 2 precursor NP_002244.1 (SEQ ID
NO: 275)

| | |
|---|---|
| 1 | mqskvllava lwlcvetraa svglpsysld lprlsiqkdi ltikanttlq itcrgqrdld |
| 61 | wlwpnnqsgs eqrvevtecs dglfcktlti pkvigndtga ykcfyretdl asviyvyvqd |
| 121 | yrspfiasvs dqhgvvyite nknktvvipc lgsisnlnvs lcarypekrf vpdgnriswd |
| 181 | skkgftipsy misyagmvfc eakindesyq simyivvvvg yriydvvlsp shgielsvge |
| 241 | klvlnctart elnvgidfnw eypsskhqhk klvnrdlktq sgsemkkfls tltidgvtrs |
| 301 | dqglytcaas sglmtkknst fvrvhekpfv afgsgmeslv eatvgervri pakylgyppp |
| 361 | eikwykngip lesnhtikag hvltimevse rdtgnytvil tnpiskekqs hvvslvvyvp |
| 421 | pqigekslis pvdsyqygtt qtltctvyai ppphhihwyw qleeecanep sqaysvtnpy |
| 481 | pceewrsved fqggnkievn knqfaliegk nktvstiviq aanvsalykc eavnkvgrge |
| 541 | rvisfhvtrg peitlqpdmq pteqesyslw ctadrstfen ltwyklgpqp lpihvgelpt |
| 601 | pvcknldtlw klnatmfsns tndilimelk naslqdqgdy vclaqdrktk krhcvvrqlt |
| 661 | vlervaptit gnlenqttsi gesievscta sgnpppqimw fkdnetlved sgivlkdgnr |
| 721 | nltirrvrke deglytcqac svlgcakvea ffiiegaqek tnleiiilvg taviamffwl |
| 781 | llviilrtvk ranggelktg ylsivmdpde lpldehcerl pydaskwefp rdrlklgkpl |
| 841 | grgafgqvie adafgidkta tcrtvavkml kegathsehr almselkili highhlnvvn |
| 901 | llgactkpgg plmvivefck fgnlstylrs krnefvpykt kgarfrqgkd yvgaipvdlk |
| 961 | rrldsitssq ssassgfvee kslsdveeee apedlykdfl tlehlicysf qvakgmefla |
| 1021 | srkcihrdla arnillsekn vvkicdfgla rdiykdpdyv rkgdarlplk wmapetifdr |
| 1081 | vytiqsdvws fgvllweifs lgaspypgvk ideefcrrlk egtrmrapdy ttpemyqtml |
| 1141 | dcwhgepsqr ptfselvehl gnllqanaqq dgkdyivlpi setlsmeeds glslptspvs |
| 1201 | cmeeeevcdp kfhydntagi sqylqnskrk srpvsvktfe dipleepevk vipddnqtds |
| 1261 | gmvlaseelk tledrtklsp sfggmvpsks resvasegsn qtsgyqsgyh sddtdttvys |
| 1321 | seeaellkli eigvqtgsta qilqpdsgtt lssppv |

Mast/stem cell growth acor receptor KIT, isoform 1 precursor NP_000213.1 (SEQ
ID NO: 276)

| | |
|---|---|
| 1 | mrgargawdf lcvlllllrv qtgssqpsys pgepsppsih pgksdlivrv gdeirllctd |
| 61 | pgfvkwtfei ldetnenkqn ewitekaeat ntgkytctnk hglsnsiyvf vrdpaklflv |
| 121 | drslygkedn dtlvrcpltd pevtnyslkg cqgkplpkdl rfipdpkagi miksvkrayh |
| 181 | rlclhcsvdq egksvlsekf ilkvrpafka vpvvsyskas yllregeeft vtctikdvss |
| 241 | svystwkren sqtklqekyn swhhgdfnye rqatltissa rvndsgvfmc yanntfgsan |
| 301 | vtttlevvdk gfinifpmin ttvfvndgen vdliveyeaf pkpehqqwiy mnrtftdkwe |
| 361 | dypksenesn iryvselhlt rlkgteggty tflvsnsdvn aaiafnvyvn tkpeiltydr |
| 421 | lvngmlqcva agfpeptidw yfcpgteqrc sasvlpvdvq tlnssgppfg klvvqssids |
| 481 | safkhngtve ckayndvgkt sayfnfafkg nnkeqihpht lftplligfv ivagmmciiv |
| 541 | miltykylqk pmyevqwkvv eeingnnyvy idptqlpydh kwefprnrls fgktlgagaf |
| 601 | gkvveatayg liksdaamtv avkmlkpsah lterealmse lkvlsylgnh mnivnllgac |
| 661 | tiggptlvit eyccygdlln flrrkrdsfi cskqedhaea alyknllhsk esscsdstne |
| 721 | ymdmkpgvsy vvptkadkrr svrigsyier dvtpaimedd elaldledll sfsyqvakgm |
| 781 | aflaskncih rdlaarnill thgritkicd fglardiknd snyvvkgnar lpvkwmapes |
| 841 | ifncvytfes dvwsygiflw elfslgsspy pgmpvdskfy kmikegfrml spehapaemy |
| 901 | dimktcwdad plkrptfkqi vqliekqise stnhiysnla ncspnrqkpv vdhsvrinsv |
| 961 | gstasssqpl lvhddv |

Mast/stem cell growth acor receptor KIT, isoform 2 precursor NP_001087241.1
(SEQ ID NO: 277)

| | |
|---|---|
| 1 | mrgargawdf lcvlllllrv qtgssqpsvs pgepsppsih pgksdlivrv gdeirllctd |
| 61 | pgfvkwtfei ldetnenkqn ewitekaeat ntgkytctnk hglsnsiyvf vrdpaklflv |
| 121 | drslygkedn dtlvrcpltd pevtnyslkg cqgkplpkdl rfipdpkagi miksvkrayh |
| 181 | r1c1hcsvdq egksvlsekf ilkvrpafka vpvvsyskas yllregeeft vtctikdvss |
| 241 | svystwkren sqtklqekyn swhhgdfnye rqatltissa rvndsgvfmc yanntfgsan |
| 301 | vtttlevvdk gfinifpmin ttvfvndgen vdliveyeaf pkpehqqwiy mnrtftdkwe |
| 361 | dypksenesn iryvselhlt rlkgteggty tflvsnsdvn aaiafnvyvn tkpeiltydr |
| 421 | lvngmlqcva agfpeptidw yfcpgteqrc sasvlpvdvq tlnssgppfg klvvqssids |
| 481 | safkhngtve ckayndvgkt sayfnfafke qihphtlftp lligfvivag mmciivmilt |
| 541 | ykylqkpmye vqwkvveein gnnyvyidpt qlpydhkwef prnrlsfgkt lgagafgkvv |
| 601 | eataygliks daamtvavkm lkpsahlter ealmselkvl sylgnhmniv nllgactigg |
| 661 | ptiviteycc ygdllnflrr krdsficskq edhaeaalyk nllhskessc sdstneymdm |
| 721 | kpgvsyvvpt kadkrrsvri gsyierdvtp aimeddelal dledllsfsy qvakgmafla |
| 781 | skncihrdla arnillthgr itkicdfgla rdikndsnyv vkgnarlpvk wmapesifnc |
| 841 | vytfesdvws ygiflwelfs lgsspypgmp vdskfykmik egfrmlspeh apaemydimk |
| 901 | tcwdadplkr ptfkqivqli ekqisestnh iysnlancsp nrqkpvvdhs vrinsvgsta |
| 961 | sssqpllvhd dv |

Plasma kallikrein isoform 1 preprotein NP_001639.1 (SEQ ID NO: 278)

| | |
|---|---|
| 1 | mwvpvvfltl svtwigaapl ilsrivggwe cekhsqpwqv lvasrgravc ggvlvhpqwv |
| 61 | ltaahcirnk svillgrhsl fhpedtgqvf qvshsfphpl ydmsllknrf lrpgddsshd |
| 121 | lmllrlsepa eltdavkvmd lptqepalgt tcyasgwgsi epeefltpkk lqcvdlhvis |
| 181 | ndvcaqvhpq kvtkfmlcag rwtggkstcs gdsggplvcn gvlqgitswg sepcalperp |
| 241 | slytkvvhyr kwikdtivan p |

Plasma kallikrein isoform 3 preprotein NP_001025218.1 (SEQ ID NO: 279)

| | |
|---|---|
| 1 | mwvpvvfltl svtwigaapl ilsrivggwe cekhsqpwqv lvasrgravc ggvlvhpqwv |
| 61 | ltaahcirnk svillgrhsl fhpedtgqvf qvshsfphpl ydmsllknrf lrpgddsshd |
| 121 | lmllrlsepa eltdavkvmd lptqepalgt tcyasgwgsi epeefltpkk lqcvdlhvis |
| 181 | ndvcaqvhpq kvtkfmlcag rwtggkstcs wviliteltm palpmvlhgs lvpwrggv |

Plasma kallikrein isoform 4 preprotein NP_001025219.1 (SEQ ID NO: 280)

| | |
|---|---|
| 1 | mwvpvvfltl svtwigaapl ilsrivggwe cekhsqpwqv lvasrgravc ggvlvhpqwv |
| 61 | ltaahcirkp gddsshdlml lrlsepaelt davkvmdlpt qepalgttcy asgwgsiepe |
| 121 | efltpkklqc vdlhvisndv caqvhpqkvt kfmlcagrwt ggkstcsgds ggplvcngvl |
| 181 | qgitswgsep calperpsly tkvvhyrkwi kdtivanp |

Tyrosine-protein kinase LCK, isoform a NP_001036236.1, NP_005347.3 (SEQ ID
NO: 281)

| | |
|---|---|
| 1 | mgcgcsshpe ddwmenidvc enchypivpl dgkgtllirn gsevrdplvt yegsnppasp |
| 61 | lqdnlvialh syepshdgdl gfekgeqlri leqsgewwka qslttgqegf ipfnfvakan |
| 121 | slepepwffk nlsrkdaerq llapgnthgs fliresesta gsfslsvrdf dqnqgevvkh |
| 181 | ykirnldngg fyispritfp glhelvrhyt nasdglctrl srpcqtqkpq kpwwedewev |
| 241 | pretlklver lgagqfgevw mgyynghtkv avkslkqgsm spdaflaean lmkqlqhqrl |
| 301 | vrlyavvtqe piyiiteyme ngslvdflkt psgikltink lldmaaqiae gmafieerny |
| 361 | ihrdlraani lvsdtlscki adfglarlie dneytarega kfpikwtape ainygtftik |
| 421 | sdvwsfgill teivthgrip ypgmtnpevi qnlergyrmv rpdncpeely qlmrlcwker |
| 481 | pedrptfdyl rsvledffta tegqyqpqp |

Tyrosine-protein kinase LCK, isoform b NP_001317397.1 (SEQ ID NO: 282)

| | |
|---|---|
| 1 | mgcgcsshpe ddwmenidvc enchypivpl dgkgtllirn gsevrdplvt yegsnppasp |
| 61 | lqdnlvialh syepshdgdl gfekgeqlri leqsgewwka qslttgqegf ipfnfvakan |
| 121 | slepepwffk nlsrkdaerq llapgnthgs fliresesta gsfslsvrdf dqnqgevvkh |
| 181 | ykirnldngg fyispritfp glhelvrhyt ryynghtkva vkslkqgsms pdaflaeanl |
| 241 | mkqlqhqrlv rlyavvtqep iyiiteymen gslvdflktp sgikltinkl ldmaaqiaeg |
| 301 | mafieernyi hrdlraanil vsdtlsckia dfglarlied neytaregak fpikwtapea |
| 361 | inygtftiks dvwsfgillt eivthgripy pgmtnpeviq nlergyrmvr pdncpeelyq |
| 421 | lmrlcwkerp edrptfdylr svledfftat egqyqpqp |

Legumain preprotein NP_001008530.1, NP_005597.3 (SEQ ID NO: 283)

| | |
|---|---|
| 1 | mvwkvavfls valgigavpi ddpedggkhw vvivagsngw ynyrhqadac hayqiihrng |
| 61 | ipdeqivvmm yddiaysedn ptpgivinrp ngtdvyqgvp kdytgedvtp qnflavlrgd |
| 121 | aeavkgigsg kvlksgpqdh vfiyftdhgs tgilvfpned lhvkdlneti hymykhkmyr |
| 181 | kmvfyieace sgsmmnhlpd ninvyattaa npressyacy ydekrstylg dwysvnwmed |
| 241 | sdvedltket lhkqyhlvks htntshvmqy gnktistmkv mqfqgmkrka sspvplppvt |
| 301 | hldltpspdv pltimkrklm ntndleesrq lteeiqrhld arhlieksvr kivsllaase |
| 361 | aeveqllser apltghscyp eallhfrthc fnwhsptyey alrhlyvlvn lcekpyplhr |
| 421 | iklsmdhvcl ghy |

Macrophage migration inhibitory factor NP_002406.1 (SEQ ID NO: 284)

| | |
|---|---|
| 1 | mpmfivntnv prasvpdgfl seltqqlaqa tgkppqyiav hvvpdqlmaf ggssepcalc |
| 61 | slhsigkigg aqnrsyskll cgllaerlri spdrvyinyy dmnaanvgwn nstfa |

MAGE family member A1 NP_004979.3 (SEQ ID NO: 285)

| | |
|---|---|
| 1 | msleqrslhc kpeealeaqq ealglvcvqa atssssplvl gtleevptag stdppqspqg |
| 61 | asafpttinf trqrqpsegs ssreeegpst scileslfra vitkkvadlv gflllkyrar |
| 121 | epvtkaemle sviknykhcf peifgkases lqlvfgidvk eadptghsyv lvtclglsyd |
| 181 | gllgdnqimp ktgfliivlv miamegghap eeeiweelsv mevydgrehs aygeprkllt |
| 241 | qdlvqekyle yrqvpdsdpa ryeflwgpra laetsyvkvl eyvikvsary rfffpslrea |
| 301 | alreeeegv |

Melanoma-associated antigen 10 NP_001011543.2, NP_001238757.1, NP_066386.2
(SEQ ID NO: 286)

| | |
|---|---|
| 1 | mprapkrqrc mpeedlqsqs etqglegaqa plaveedass ststsssfps sfpsssssss |
| 61 | sscyplipst peevsaddet pnppqsaqia csspsvvasl pldqsdegss sqkeespstl |
| 121 | qvlpdseslp rseidekvtd lvqfllfkyq mkepitkaei lesvirnyed hfpllfseas |
| 181 | ecmllvfgid vkevdptghs fvlvtslglt ydgmlsdvqs mpktgilili lsiifiegyc |
| 241 | tpeeviweal nmmglydgme hliygeprkl ltqdwvqeny leyrqvpgsd paryeflwgp |
| 301 | rahaeirkms llkflakvng sdprsfplwy eealkdeeer aqdriattdd ttamasasss |
| 361 | atgsfsype |

Melanoma-associated antigen 12 NP_001159858.1, NP_001159859.1, NP_005358.2
(SEQ ID NO: 287)

| | |
|---|---|
| 1 | mpleqrsqhc kpeegleaqg ealglvgaqa pateeqetas ssstlvevtl revpaaesps |
| 61 | pphspqgast lpttinytlw sqsdegssne eqegpstfpd letsfqvals rkmaelvhfl |
| 121 | llkyrarepf tkaemlgsvi rnfqdffpvi fskaseylql vfgievvevv righlyilvt |
| 181 | clglsydgll gdnqivpktg lliivlaiia kegdcapeek iweelsvlea sdgredsvfa |
| 241 | hprklltqdl vqenyleyrq vpgsdpacye flwgpralve tsyvkvlhhl lkisggphis |
| 301 | ypplhewafr egee |

Melanoma-associated antigen 2 NP_001269430.1, NP_001269431.1, NP_001269433.1,
NP_001269434.1, NP_005352.1, NP_786884.1, NP_786885.1 (SEQ ID NO: 288)

| | |
|---|---|
| 1 | mpleqrsqhc kpeeglearg ealglvgaqa pateeqqtas ssstlvevtl gevpaadsps |
| 61 | pphspqgass fsttinytlw rqsdegssnq eeegprmfpd lesefqaais rkmvelvhfl |
| 121 | llkyrarepv tkaemlesvl rncqdffpvi fskaseylql vfgievvevv pishlyilvt |
| 181 | clglsydgll gdnqvmpktg lliivlaiia iegdcapeek iweelsmlev fegredsvfa |
| 241 | hprkllmqdl vqenyleyrq vpgsdpacye flwgpralie tsyvkvlhht lkiggephis |
| 301 | ypplheralr egee |

MAGE family member A3 NP_005353.1 (SEQ ID NO: 289)

| | |
|---|---|
| 1 | mpleqrsqhc kpeeglearg ealglvgaqa pateeqeaas ssstlvevtl gevpaaespd |
| 61 | ppqspqgass lpttmnyplw sqsyedssnq eeegpstfpd lesefqaals rkvaelvhfl |
| 121 | llkyrarepv tkaemlgsvv gnwqyffpvi fskassslql vfgielmevd pighlyifat |
| 181 | clglsydgll gdnqimpkag lliivlaiia regdcapeek iweelsvlev fegredsilg |
| 241 | dpkklltqhf vqenyleyrq vpgsdpacye flwgpralve tsyvkvlhhm vkisggphis |
| 301 | ypplhewvlr egee |

Melanoma-associated antigen 4 NP_001011548.1, NP_001011549.1, NP_001011550.1,
NP_002353.3 (SEQ ID NO: 290)

| | |
|---|---|
| 1 | msseqksqhc kpeegveaqe ealglvgaqa ptteeqeaav ssssplvpgt leevpaaesa |
| 61 | gppqspqgas alpttisftc wrqpnegsss qeeegpstsp daeslfreal snkvdelahf |
| 121 | llrkyrakel vtkaemlerv iknykrcfpv ifgkaseslk mifgidvkev dpasntytlv |
| 181 | tclglsydgl lgnnqifpkt glliivlgti amegdsasee eiweelgvmg vydgrehtvy |
| 241 | geprklltqd wvqenyleyr qvpgsnpary eflwgprala etsyvkvleh vvrvnarvri |
| 301 | aypslreaal leeeegv |

Melanoma-associated antigen 6 NP_005354.1, NP_787064.1 (SEQ ID NO: 291)

| | |
|---|---|
| 1 | mpleqrsqhc kpeeglearg ealglvgaqa pateeqeaas ssstivevtl gevpaaespd |
| 61 | ppqspqgass lpttmnyplw sqsyedssnq eeegpstfpd lesefqaals rkvaklvhfl |
| 121 | llkyrarepv tkaemlgsvv gnwqyffpvi fskasdslql vfgielmevd pighvyifat |
| 181 | clglsydgll gdnqimpktg fliiilaiia kegdcapeek iweelsvlev fegredsifg |
| 241 | dpkklltqyf vqenyleyrq vpgsdpacye flwgpralie tsyvkvlhhm vkisggpris |
| 301 | ypllhewalr egee |

Melanoma-associated antigen 9 NP_005356.1 (SEQ ID NO: 292)

| | |
|---|---|
| 1 | msleqrsphc kpdedleaqg edlglmgaqe ptgeeeetts ssdskeeevs aagsssppqs |
| 61 | pqggasssis vyytlwsqfd egsssqeeee psssvdpaql efmfqealkl kvaelvhfll |
| 121 | hkyrvkepvt kaemlesvik nykryfpvif gkasefmqvi fgtdvkevdp aghsyilvta |
| 181 | lglscdsmlg dghsmpkaal liivlgvilt kdncapeevi wealsvmgvy vgkehmfyge |
| 241 | prklltqdwv qenyleyrqv pgsdpahyef lwgskahaet syekvinylv mlnarepicy |
| 301 | pslyeevlge eqegv |

Melanoma-associated antigen C2 NP_057333.1 (SEQ ID NO: 293)

| | |
|---|---|
| 1 | mppvpgvpfr nvdndsptsv eledwvdaqh ptdeeeeeas sasstlylvf spssfstsss |
| 61 | lilggpeeee vpsgvipnlt esipssppqg ppqgpsqspl ssccssfsws sfseesssqk |
| 121 | gedtgtcqgl pdsessftyt ldekvaelve flllkyeaee pvteaemlmi vikykdyfpv |
| 181 | ilkrarefme llfglaliev gpdhfcvfan tvgltdegsd degmpensll iiilsvifik |
| 241 | gncaseeviw evlnavgvya grehfvygep relltkvwvq ghyleyrevp hssppyyefl |
| 301 | wgprahsesi kkkvleflak lnntvpssfp swykdalkdv eervqatidt addatvmase |
| 361 | slsvmssnvs fse |

Melanoma-associated antigen D1, isoform a NP_001005333.1 (SEQ ID NO: 294)

| | |
|---|---|
| 1 | maqkmdcgag llgfqnpdac ravchplpqp pastlplsaf pticdppysq lrdppavlsc |
| 61 | yctplgaspa paeasvedsa llmqtlmeai qiseapptnq ataaaspqss qpptanemad |
| 121 | iqvsaaaarp ksafkvqnat tkgpngvydf sqahnakdvp ntqpkaafks qnatpkgpna |
| 181 | aydfsqaatt gelaanksem afkaqnattk vgpnatynfs qslnandlan srpktpfkaw |
| 241 | ndttkaptad tqtqnvnqak matsqadiet dpgisepdga taqtsadgsq aqnlesrtii |
| 301 | rgkrtrkinn lnveenssgd qrraplaagt wrsapvpvtt qnppgappnv lwqtplawqn |
| 361 | psgwqnqtar qtpparqspp arqtppawqn pvawqnpviw pnpviwqnpv iwpnpivwpg |
| 421 | pvvwpnplaw qnppgwqtpp gwqtppgwqg ppdwqgppdw plppdwplpp dwplptdwpl |
| 481 | ppdwipadwp ippdwqnlrp spnlrpspns rasqnpgaaq prdvallqer anklvkylml |
| 541 | kdytkvpikr semlrdiire ytdvypeiie racfvlekkf giqlkeidke ehlyilistp |
| 601 | eslagilgtt kdtpklglll vilgvifmng nraseavlwe alrkmglrpg vrhpllgdlr |
| 661 | klltyefvkq kyldyrrvpn snppeyeflw glrsyhetsk mkvlrfiaev qkrdprdwta |
| 721 | qfmeaadeal daldaaaaea earaeartrm gigdeaysgp wswddiefel ltwdeegdfg |
| 781 | dpwsripftf waryhqnars rfpqtfagpi igpggtasan faanfgaigf fwve |

Melanoma-associated antigen D1, isoform b NP_001005332.1, NP_008917.3 (SEQ ID
NO: 295)

| | |
|---|---|
| 1 | maqkmdcgag llgfqaeasv edsallmqtl meaiqiseap ptnqataaas pqssqpptan |
| 61 | emadiqvsaa aarpksafkv qnattkgpng vydfsqahna kdvpntqpka afksqnatpk |
| 121 | gpnaaydfsq aattgelaan ksemafkaqn attkvgpnat ynfsqslnan dlansrpktp |
| 181 | fkawndttka ptadtqtqnv nqakmatsqa dietdpgise pdgataqtsa dgsqaqnles |
| 241 | rtiirgkrtr kinnlnveen ssgdqrrapl aagtwrsapv pvttqnppga ppnvlwqtpl |
| 301 | awqnpsgwqn qtarqtppar qspparqtpp awqnpvawqn pviwpnpviw qnpviwpnpi |
| 361 | vwpgpvvwpn plawqnppgw qtppgwqtpp gwqgppdwqg ppdwplppdw plppdwplpt |
| 421 | dwplppdwip adwpippdwq nlrpspnlrp spnsrasqnp gaaqprdval lqeranklvk |
| 481 | ylmlkdytkv pikrsemlrd iireytdvyp eiieracfvl ekkfgiqlke idkeehlyil |
| 541 | istpeslagi lgttkdtpkl glllvilgvi fmngnrasea vlwealrkmg lrpgvrhpll |
| 601 | gdlrklltye fvkqkyldyr rvpnsnppey eflwglrsyh etskmkvlrf iaevqkrdpr |
| 661 | dwtaqfmeaa dealdaldaa aaeaearaea rtrmgigdea vsgpwswddi efelltwdee |
| 721 | gdfgdpwsri pftfwaryhq narsrfpqtf agpiigpggt asanfaanfg aigffwve |

Mitogen-activated protein kinase kinase kinase 5 NP_005914.1 (SEQ ID NO: 296)

| | |
|---|---|
| 1 | msteadegit fsvppfapsg fctipeggic rrggaaavge geehqlpppp pgsfwnvesa |
| 61 | aapgigcpaa tssssatrgr gssvgggsrr ttvayvinea sqgqlvvaes ealqslreac |
| 121 | etvgatletl hfgkldfget tvldrfynad iavvemsdaf rqpslfyhlg vresfsmann |
| 181 | iilycdtnsd slqslkeiic qkntmctgny tfvpymitph nkvyccdssf mkgltelmqp |
| 241 | nfelllgpic lplvdrfiql lkvagasssq yfresilndi rkarnlytgk elaaelarir |
| 301 | qrvdnievlt adivinllls yrdiqdydsi vklvetlekl ptfdlashhh vkfhyafaln |
| 361 | rrnlpgdrak aldimipmvq segqvasdmy clvgriykdm fldsnftdte srdhgaswfk |
| 421 | kafeseptlq sginyavlll aaghqfessf elrkvgvkls sllgkkgnle klqsywevgf |
| 481 | flgasvland hmrviqasek lfklktpawy lksivetili ykhfvkltte qpvakqelvd |
| 541 | fwmdflveat ktdvtvvrfp vlileptkiy qpsylsinne veektisiwh vlpddkkgih |
| 601 | ewnfsassvr gvsiskfeer ccflyvlhns ddfqiyfcte lhckkffemv ntiteekgrs |
| 661 | teegdcesdl leydyeyden gdrvvlgkgt ygivyagrdl snqvriaike iperdsrysq |
| 721 | plheeialhk hlkhknivqy lgsfsengfi kifmeqvpgg slsallrskw gplkdneqti |
| 781 | gfytkqileg lkylhdnqiv hrdikgdnvl intysgvlki sdfgtskrla ginpctetft |
| 841 | gtlqymapei idkgprgygk aadiwslgct iiematgkpp fyelgepqaa mfkvgmfkvh |
| 901 | peipesmsae akafilkcfe pdpdkracan dllvdeflkv sskkkktqpk lsalsagsne |
| 961 | ylrsislpvp vlvedtssss eygsyspdte lkvdpfsfkt rakscgerdv kgirtlflgi |
| 1021 | pdenfedhsa ppspeekdsg ffmlrkdser ratlhrilte dqdkivrnlm eslaqgaeep |
| 1081 | klkwehittl iaslrefvrs tdrkiiattl sklkleldfd shgisqvqvv lfgfqdavnk |
| 1141 | vlrnhnikph wmfaldsiir kavqtaitil vpelrphfsl asesdtadqe dldveddhee |
| 1201 | qpsnqtvrrp qaviedavat sgvstlsstv shdsqsahrs lnvqlgrmki etnrlleelv |
| 1261 | rkekelqall hraieekdqe ikhlklksqp ieipelpvfh lnssgtnted seltdwlrvn |
| 1321 | gadedtisrf laedytlldv lyyvtrddlk clrlrggmlc tlwkaiidfr nkqt |

Mitogen-activated protein kinase kinase kinase 9, isoform 1 NP_149132.2 (SEQ
ID NO: 297)

| | |
|---|---|
| 1 | mepsrallgc lasaaaaapp gedgagagae eeeeeeeeaa aavgpgelgc daplpywtav |
| 61 | feyeaagede ltlrlgdvve vlskdsqvsg degwwtgqln qrvgifpsny vtprsafssr |
| 121 | cqpggedpsc yppiqlleid faeltleeii giggfgkvyr afwigdevav kaarhdpded |
| 181 | isqtienvrq eaklfamlkh pniialrgvc lkepnlclvm efarggpinr vlsgkrippd |
| 241 | ilvnwavqia rgmnylhdea ivpiihrdlk ssnililqkv engdlsnkil kitdfglare |
| 301 | whrttkmsaa gtyawmapev irasmfskgs dvwsygvllw elltgevpfr gidglavayg |
| 361 | vamnklalpi pstcpepfak lmedcwnpdp hsrpsftnil dqlttieesg ffempkdsfh |
| 421 | clqdnwkhei qemfdqlrak ekelrtweee ltraalqqkn qeellrrreq elaereidil |
| 481 | erelniiihq lcqekprvkk rkgkfrksrl klkdgnrisl psdfqhkftv gasptmdkrk |
| 541 | slinsrsspp asptiiprlr aiqltpgess ktwgrssvvp keegeeeekr apkkkgrtwg |
| 601 | pgtlgqkela sgdegspqrr ekanglstps esphfhlglk slvdgykqws ssapnlvkgp |
| 661 | rsspalpgft slmemallaa swvvpidiee dedsegpgsg esrlqhspsq sylcipfprg |
| 721 | edqdqpssdq iheeptpvns atstpqltpt nslkrggahh rrcevallgc gavlaatglg |
| 781 | fdlleagkcq llpleepepp areekkrreg lfqrssrprr stsppsrklf kkeepmlllg |
| 841 | dpsasltlls lssisecnst rsllrsdsde ivvyempvsp veapplspct hnplvnvrve |
| 901 | rfkrdpnqsl tpthvtlttp sqpsshrrtp sdgalkpetl lasrspssng lspspgagml |
| 961 | ktpspsrdpg efprlpdpnv vfpptprrwn tqqdstlerp ktleflprpr psanrqrldp |
| 1021 | wwfvspshar stspanssst etpsnldscf asssstveer pglpallpfq agplpptert |
| 1081 | lldldaegqs qdstvplcra elnthrpapy eiqqefws |

Mitogen-activated protein kinase kinase kinase 9, isoform 2 NP_001271159.1
(SEQ ID NO: 298)

| | |
|---|---|
| 1 | mepsrallgc lasaaaaapp gedgagagae eeeeeeeeaa aavgpgelgc daplpywtav |
| 61 | feyeaagede ltlrlgdvve vlskdsqvsg degwwtgqln qrvgifpsny vtprsafssr |
| 121 | cqpggedpsc yppiqlleid faeltleeii giggfgkvyr afwigdevav kaarhdpded |
| 181 | isqtienvrq eaklfamlkh pniialrgvc lkepnlclvm efarggpinr vlsgkrippd |
| 241 | ilvnwavqia rgmnylhdea ivpiihrdlk ssnililqkv engdlsnkil kitdfglare |
| 301 | whrttkmsaa gtyawmapev irasmfskgs dvwsygvllw elltgevpfr gidglavayg |
| 361 | vamnklalpi pstcpepfak lmedcwnpdp hsrpsftnil dqlttieesg ffempkdsfh |
| 421 | clqdnwkhei qemfdqlrak ekelrtweee ltraalqqkn qeellrrreq elaereidil |
| 481 | erelniiihq lcqekprvkk rkgkfrksrl klkdgnrisl psdfqhkftv qasptmdkrk |
| 541 | slinsrsspp asptiiprlr aiqltpgess ktwgrssvvp keegeeeekr apkkkgrtwg |
| 601 | pgtlgqkela sgdegspqrr ekanglstps esphfhlglk slvdgykqws ssapnlvkgp |
| 661 | rsspalpgft slmemededs egpgsgesrl qhspsqsylc ipfprgedgd gpssdgihee |
| 721 | ptpvnsatst pqltptnslk rggahhrrce vallgcgavl aatglgfdll eagkcqllpl |
| 781 | eepepparee kkrreglfqr ssrprrstsp psrklfkkee pmlllgdpsa sltllslssi |
| 841 | secnstrsll rsdsdeivvy empvspveap plspcthnpl vnvrverfkr dpnqsltpth |
| 901 | vtlttpsqps shrrtpsdga lkpetllasr spssnglsps pgagmlktps psrdpgefpr |
| 961 | lpdpnvvfpp tprrwntqqd stlerpktle flprprpsan rqrldpwwfv spsharstsp |
| 1021 | anssstetps nldscfasss stveerpglp allpfqagpl pptertlldl daegqsqdst |
| 1081 | vplcraelnt hrpapyeiqq efws |

Mitogen-activated protein kinase kinase kinase 9, isoform 3 NP_001271160.1
(SEQ ID NO: 299)

| | |
|---|---|
| 1 | meltgleval vlilqkveng dlsnkilkit dfglarewhr ttkmsaagty awmapevira |
| 61 | smfskgsdvw sygvllwell tgevpfrgid glavaygvam nklalpipst cpepfaklme |
| 121 | dcwnpdphsr psftnildql ttieesgffe mpkdsfhclq dnwkheiqem fdqlrakeke |
| 181 | lrtweeeltr aalqqknqee llrrreqela ereidilere lniiihqlcq ekprvkkrkg |
| 241 | kfrksrlklk dgnrislpsd fqhkftvqas ptmdkrksli nsrssppasp tiiprlraiq |
| 301 | cetvsqiswg qntqghlspa lsshrlvqac sihnfchlss tmciymhilt pgessktwgr |
| 361 | ssvvpkeege eeekrapkkk grtwgpgtlg qkelasgdeg lkslvdgykq wsssapnlvk |
| 421 | gprsspalpg ftslmemall aaswvvpidi eededsegpg sgesrlqhsp sqsylcipfp |
| 481 | rgedgdgpss dgiheeptpv nsatstpqlt ptnslkrgga hhrrcevall gcgavlaatg |
| 541 | lgfdlleagk cqllpleepe ppareekkrr eglfqrssrp rrstsppsrk lfkkeepmll |
| 601 | lgdpsasltl lslssisecn strsllrsds deivvyempv spveapplsp cthnplvnvr |
| 661 | verfkrdpnq sltpthvtlt tpsqpsshrr tpsdgalkpe tllasrspss nglspspgag |
| 721 | mlktpspsrd pgefprlpdp nvvfpptprr wntqqdstle rpktleflpr prpsanrqrl |
| 781 | dpwwfvspsh arstspanss stetpsnlds cfasssstve erpglpallp fqagplppte |
| 841 | rtlldldaeg qsqdstvplc raelnthrpa pyeiqqefws |

Mitogen-activated protein kinase kinase kinase 9, isoform 4 NP_001271161.1
(SEQ ID NO: 300)

| | |
|---|---|
| 1 | msaagtyawm apevirasmf skgsdvwsyg vllwelltge vpfrgidgla vaygvamnkl |
| 61 | alpipstcpe pfaklmedcw npdphsrpsf tnildqltti eesgffempk dsfhclqdnw |
| 121 | kheiqemfdq lrakekelrt weeeltraal qqknqeellr rreqelaere idilerelni |
| 181 | iihqlcqekp rvkkrkgkfr ksrlklkdgn rislpsdfqh kftvqasptm dkrkslinsr |
| 241 | ssppasptii prlraiqcet vsgiswgqnt qghlspalss hrlvqacsih nfchlsstmc |
| 301 | iymhiltpge ssktwgrssv vpkeegeeee krapkkkgrt wgpgtlgqke lasgdeglks |
| 361 | lvdgykqwss sapnlvkgpr sspalpgfts lmemallaas wvvpidieed edsegpgsge |
| 421 | srlqhspsqs ylcipfprge dgdgpssdgi heeptpvnsa tstpqltptn slkrggahhr |
| 481 | rcevallgcg avlaatglgf dlleagkcql lpleepeppa reekkrregl fqrssrprrs |
| 541 | tsppsrklfk keepmlllgd psasltllsl ssisecnstr sllrsdsdei vvyempvspv |
| 601 | eapplspcth nplvnvrver fkrdpnqslt pthvtlttps qpsshrrtps dgalkpetll |
| 661 | asrspssngl spspgagmlk tpspsrdpge fprlpdpnvv fpptprrwnt qqdstlerpk |
| 721 | tleflprprp sanrqrldpw wfvspshars tspanssste tpsnldscfa sssstveerp |
| 781 | glpallpfqa gplpptertl ldldaeggsq dstvplcrae lnthrpapye iqqefws |

Mitogen-activated protin kinase 1 NP_002736.3, NP_620407.1 (SEQ ID NO: 301)

| | |
|---|---|
| 1 | maaaaaagag pemvrgqvfd vgprytnlsy igegaygmvc saydnvnkvr vaikkispfe |
| 61 | hqtycqrtlr eikillrfrh eniigindii raptieqmkd vyivqdlmet dlykllktqh |
| 121 | lsndhicyfl yqilrglkyi hsanvlhrdl kpsnlllntt cdlkicdfgl arvadpdhdh |
| 181 | tgflteyvat rwyrapeiml nskgytksid iwsvgcilae mlsnrpifpg khyldqlnhi |
| 241 | lgilgspsqe dlnciinlka rnyllslphk nkvpwnrlfp nadskaldll dkmltfnphk |
| 301 | rieveqalah pyleqyydps depiaeapfk fdmelddlpk eklkelifee tarfqpgyrs |

Melan-A NP_005502.1 (SEQ ID NO: 302)

| | |
|---|---|
| 1 | mpredahfiy gypkkghghs yttaeeaagi giltvilgvl lligcwycrr rngyralmdk |
| 61 | slhvgtqcal trrcpqegfd hrdskvslqe kncepvvpna ppayeklsae qspppysp |

Melanotransferrin, isoform 1 preprotein NP_005920.2 (SEQ ID NO: 303)

| | |
|---|---|
| 1 | mrgpsgalwl llalrtvlgg mevrwcatsd peqhkcgnms eafreagiqp sllcvrgtsa |
| 61 | dhcvqliaaq eadaitldgg aiyeagkehg lkpvvgevyd qevgtsyyav avvrrsshvt |
| 121 | idtlkgvksc htginrtvgw nvpvgylves grlsvmgcdv lkaysdyfgg scvpgagets |
| 181 | yseslcrlcr gdssgegvcd kspleryydy sgafrclaeg agdvafvkhs tvlentdgkt |
| 241 | lpswgqalls qdfellcrdg sradvtewrq chlarvpaha vvvradtdgg lifrllnegq |
| 301 | rlfshegssf qmfsseaygq kdllfkdsts elvpiatqty eawlgheylh amkgllcdpn |
| 361 | rlppylrwcv lstpeiqkcg dmavafrrqr lkpeiqcvsa kspqhcmeri qaeqvdavtl |
| 421 | sgediytagk tyglvpaage hyapedssns yyvvavvrrd sshaftldel rgkrschagf |
| 481 | gspagwdvpv galiqrgfir pkdcdvltav seffnascvp vnnpknypss lcalcvgdeq |
| 541 | grnkcvgnsq eryygyrgaf rclvenagdv afvrhttvfd ntnghnsepw aaelrsedye |
| 601 | llcpngarae vsqfaacnla qipphavmvr pdtniftvyg lldkaqdlfg ddhnkngfkm |
| 661 | fdssnyhgqd llfkdatvra vpvgekttyr gwlgldyvaa legmssqqcs gaaapapgap |
| 721 | llplllpala arllppal |

Melanotransferrin, isoform 2 precursor NP_201573.1 (SEQ ID NO: 304)

| | |
|---|---|
| 1 | mrgpsgalwl llalrtvlgg mevrwcatsd peqhkcgnms eafreagiqp sllcvrgtsa |
| 61 | dhcvqliaaq eadaitldgg aiyeagkehg lkpvvgevyd qevgtsyyav avvrrsshvt |
| 121 | idtlkgvksc htginrtvgw nvpvgylves grlsvmgcdv lkaysdyfgg scvpgagets |
| 181 | yseslcrlcr gdssgegvcd kspleryydy sgafrclaeg agdvafvkhs tvlentdesp |
| 241 | srrqtwtrse eeegecpahe earrtmrssa gqawkwapvh rpqdesdkge fgkraksrdm |
| 301 | lg |

Baculoviral IAP repeat containing 7, isoform alpha NP_647478.1 (SEQ ID NO:
305)

| | |
|---|---|
| 1 | mgpkdsakcl hrgpqpshwa agdgptqerc gprslgspvl gldtcrawdh vdgqilgqlr |
| 61 | plteeeeeeg agatlsrgpa fpgmgseelr lasfydwplt aevppellaa agffhtghqd |
| 121 | kvrcffcygg lqswkrgddp wtehakwfps cqfllrskgr dfvhsvqeth sqllgswdpw |
| 181 | eepedaapva psvpasgype lptprrevqs esagepggvs paeaqrawwv leppgardve |
| 241 | aqlrrlqeer tckvcldrav sivfvpcghl vcaecapglq lcpicrapvr srvrtfls |

Baculoviral IAP repeat containing 7, isoform beta NP_071444.1 (SEQ ID NO:
306)

| | |
|---|---|
| 1 | mgpkdsakcl hrgpqpshwa agdgptqerc gprslgspvl gldtcrawdh vdgqilgqlr |
| 61 | plteeeeeeg agatlsrgpa fpgmgseelr lasfydwplt aevppellaa agffhtghqd |
| 121 | kvrcffcygg lqswkrgddp wtehakwfps cqfllrskgr dfvhsvqeth sqllgswdpw |
| 181 | eepedaapva psvpasgype lptprrevqs esaqepgard veaqlrrlqe ertckvcldr |
| 241 | aysivfvpcg hlvcaecapg lqlcpicrap vrsrvrtfls |

Neutrophil collagenase, isoform 1 preprotein NP_002415.1 (SEQ ID NO: 307)

| | |
|---|---|
| 1 | mfslktlpfl lllhvqiska fpvsskeknt ktvqdylekf yqlpsnqyqs trkngtnviv |
| 61 | eklkemqrff glnvtgkpne etldmmkkpr cgvpdsggfm ltpgnpkwer tnltyrirny |
| 121 | tpqlseaeve raikdafelw svaspliftr isqgeadini afyqrdhgdn spfdgpngil |
| 181 | ahafqpgqgi ggdahfdaee twtntsanyn lflvaahefg hslglahssd pgalmypnya |
| 241 | fretsnyslp qddidgiqai yglssnpiqp tgpstpkpcd psltfdaitt lrgeilffkd |
| 301 | ryfwrrhpql qrvemnfisl fwpslptgiq aayedfdrdl iflfkgnqyw alsgydilqg |
| 361 | ypkdisnygf pssvqaidaa vfyrsktyff vndqfwrydn qrqfmepgyp ksisgafpgi |
| 421 | eskvdavfqq ehffhvfsgp ryyafdliaq rvtrvargnk wlncryg |

Neutrophil collagenase, isoform 2 NP_001291370.1, NP_001291371.1 (SEQ ID NO:
308)

| | |
|---|---|
| 1 | mgqipqeksi ndylekfyql psnqyqstrk ngtnvivekl kemqrffgln vtgkpneetl |
| 61 | dmmkkprcgv pdsggfmltp gnpkwertnl tyrirnytpq lseaeverai kdafelwsva |
| 121 | spliftrisq geadiniafy qrdhgdnspf dgpngilaha fqpgqgiggd ahfdaeetwt |
| 181 | ntsanynlfl vaahefghsl glahssdpga lmypnyafre tsnyslpqdd idgiqaiygl |
| 241 | ssnpiqptgp stpkpcdpsl tfdaittlrg eilffkdryf wrrhpqlqrv emnfislfwp |
| 301 | slptgiqaay edfdrdlifl fkgnqywals gydilqgypk disnygfpss vqaidaavfy |
| 361 | rsktyffvnd qfwrydnqrq fmepgypksi sgafpgiesk vdavfqqehf fhvfsgpryy |
| 421 | afdliaqrvt rvargnkwln cryg |

Mesothelin, isoform 1 preprotein NP_001170826.1, NP_005814.2 (SEQ ID NO: 309)

| | |
|---|---|
| 1 | malptarpll gscgtpalgs llfllfslgw vqpsrtlage tgqeaapldg vlanppniss |
| 61 | lsprqllgfp caevsglste rvrelavala qknvklsteq lrclahrlse ppedldalpl |
| 121 | dlllflnpda fsgpqactrf fsritkanvd llprgaperq rllpaalacw gvrgsllsea |
| 181 | dvralgglac dlpgrfvaes aevllprlvs cpgpldqdqq eaaraalqgg gppygppstw |
| 241 | systmdalrg llpvlgqpii rsipqgivaa wrqrssrdps wrqpertilr prfrrevekt |
| 301 | acpsgkkare ideslifykk weleacvdaa llatqmdrvn aipftyeqld vlkhkldely |
| 361 | pggypesviq hlgylflkms pedirkwnvt sletlkalle vnkghemspq vatlidrfvk |
| 421 | grgqldkdtl dtltafypgy lcslspeels svppssiwav rpqdldtcdp rqldvlypka |
| 481 | rlafqnmngs eyfvkiqsfl ggaptedlka lsqqnvsmdl atfmklrtda vlpltvaevq |
| 541 | kllgphvegl kaeerhrpvr dwilrqrqdd ldtlglglqg gipngylvld lsmqealsgt |
| 601 | pcllgpgpvl tvlalllast la |

Mesothelin, isoform 2 preprotein NP_037536.2 (SEQ ID NO: 310)

| | |
|---|---|
| 1 | malptarpll gscgtpalgs llfllfslgw vqpsrtlage tgqeaapldg vlanppniss |
| 61 | lsprqllgfp caevsglste rvrelavala qknvklsteq lrclahrlse ppedldalpl |
| 121 | dlllflnpda fsgpqactrf fsritkanvd llprgaperq rllpaalacw gvrgsllsea |
| 181 | dvralgglac dlpgrfvaes aevllprlvs cpgpldqdqq eaaraalqgg gppygppstw |
| 241 | systmdalrg llpvlgqpii rsipqgivaa wrqrssrdps wrqpertilr prfrrevekt |
| 301 | acpsgkkare ideslifykk weleacvdaa llatqmdrvn aipftyeqld vlkhkldely |
| 361 | pggypesviq hlgylflkms pedirkwnvt sletlkalle vnkghemspq aprrplpqva |
| 421 | tlidrfvkgr gqldkdtldt ltafypgylc slspeelssv ppssiwavrp qdldtcdprq |
| 481 | ldvlypkarl afqnmngsey fvkiqsflgg aptedlkals qqnvsmdlat fmklrtdavl |
| 541 | pltvaevqkl lgphveglka eerhrpvrdw ilrqrqddld tlglglqggi pngylvldls |
| 601 | mqealsgtpc llgpgpvltv lalllastla |

Mucin-1, isoform 1 precursor NP_002447.4 (SEQ ID NO: 311)

| | |
|---|---|
| 1 | mtpgtqspff llllltvltv vtgsghasst pggeketsat qrssvpsste knalstgvsf |
| 61 | fflsfhisnl qfnssledps tdyyqelqrd isemflqiyk qggflglsni kfrpgsvvvq |
| 121 | ltlafregti nvhdvetqfn qykteaasry nitisdvsys dvpfpfsaqs gagvpgwgia |
| 181 | llvlvcvlva laivyliala vcqcrrknyg qldifpardt yhpmseypty hthgryvpps |
| 241 | stdrspyekv sagnggssls ytnpavaats anl |

Mucin-1, isoform 2 precursor NP_001018016.1 (SEQ ID NO: 312)

| | |
|---|---|
| 1 | mtpgtqspff llllltvlta ttapkpatvv tgsghasstp ggeketsatq rssvpsstek |
| 61 | nafnssledp stdyyqelqr disemflqiy kqggflglsn ikfrpgsvvv qltlafregt |
| 121 | invhdvetqf nqykteaasr ynltisdvsv sdvpfpfsaq sgagvpgwgi allvlvcvlv |
| 181 | alaivylial avcqcrrkny gqldifpard tyhpmseypt yhthgryvpp sstdrspyek |
| 241 | vsagnggssl sytnpavaat sanl |

Mucin-1, isoform 3 precursor NP_001018017.1 (SEQ ID NO: 313)

| | |
|---|---|
| 1 | mtpgtqspff llllltvltv vtgsghasst pggeketsat qrssvpsste knafnssled |
| 61 | pstdyyqelq rdisemflqi ykqggflgls nikfrpgsvv vqltlafreg tinvhdvetq |
| 121 | fnqykteaas rynitisdvs vsdvpfpfsa qsgagvpgwg iallvlvcvl valaivylia |
| 181 | lavcqcrrkn ygqldifpar dtyhpmseyp tyhthgryvp psstdrspye kvsagnggss |
| 241 | lsytnpavaa tsanl |

Mucin-1, isoform 5 precursor NP_001037855.1 (SEQ ID NO: 314)

| | |
|---|---|
| 1 | mtpgtqspff llllltvltv vtgsghasst pggeketsat qrssvpsste knaipapttt |
| 61 | kscretflkc fcrfinkgvf waspilssvs dvpfpfsaqs gagvpgwgia llvlvcvlva |
| 121 | laivyliala vcqcrrknyg qldifpardt yhpmseypty hthgryvpps stdrspyekv |
| 181 | sagnggssls ytnpavaats anl |

Mucin-1, isoform 6 precursor NP_001037856.1 (SEQ ID NO: 315)

| | |
|---|---|
| 1 | mtpgtqspff llllltvltv vtgsghasst pggeketsat qrssvpsste knafnssled |
| 61 | pstdyyqelq rdisemavcq crrknygqld ifpardtyhp mseyptyhth gryvppsstd |
| 121 | rspyekvsag nggsslsytn pavaatsanl |

Mucin-1, isoform 7 precursor NP_001037857.1 (SEQ ID NO: 316)

| | |
|---|---|
| 1 | mtpgtqspff llllltvlta ttapkpatvv tgsghasstp ggeketsatq rssvpsstek |
| 61 | nafnssledp stdyyqelqr disemavcqc rrknygqldi fpardtyhpm seyptyhthg |
| 121 | ryvppsstdr spyekvsagn ggsslsytnp avaatsanl |

Mucin-1, isoform 8 precursor NP_001037858.1 (SEQ ID NO: 317)

| | |
|---|---|
| 1 | mtpgtqspff llllltvltv vtgsghasst pggeketsat qrssvpsste knaipapttt |
| 61 | kscretflkc fcrfinkgvf waspilssvw gwgarlghra agaglcsgca ghclshclgc |
| 121 | lsvppkelra aghlsspgyl psyervphlp hpwalcap |

Mucin-1, isoform 9 precursor NP_001191214.1 (SEQ ID NO: 318)

| | |
|---|---|
| 1 | mtpgtqspff llllltvltv vtgsghasst pggeketsat qrssvpsste knavsmtssv |
| 61 | lsshspgsgs sttqgqdvtl apatepasgs aatwgqdvts vpvtrpalgs ttppahdvts |
| 121 | apdnkpapgs tappahgvts apdtrpapgs tappahgvts apdnrpalgs tappvhnvts |
| 181 | asgsasgsas tlvhngtsar atttpaskst pfsipshhsd tpttlashst ktdassthhs |
| 241 | tvppltssnh stspqlstgv sffflsfhis nlqfnssled pstdyyqelq rdisemflqi |
| 301 | ykqggflgls nikfrpgsvv vqltlafreg tinvhdvetq fnqykteaas rynltisdvs |
| 361 | vsdvpfpfsa qsgagvpgwg iallvlvcvl valaivylia lavcqcrrkn ygqldifpar |
| 421 | dtyhpmseyp tyhthgryvp psstdrspye kvsagnggss lsytnpavaa tsanl |

Mucin-1, isoform 10 precursor NP_001191215.1 (SEQ ID NO: 319)

| | |
|---|---|
| 1 | mtpgtqspff llllltvlta ttapkpatvv tgsghasstp ggeketsatq rssvpsstek |
| 61 | naysmtssvl sshspgsgss ttqgqdvtla patepasgsa atwgqdvtsv pvtrpalgst |
| 121 | tppandvtsa pdnkpapgst appahgvtsa pdtrpapgst appahgvtsa pdnrpalgst |
| 181 | appvhnvtsa sgsasgsast lvhngtsara tttpaskstp fsipshhsdt pttlashstk |
| 241 | tdassthhst vppltssnhs tspqlstgvs ffflsfhisn lqfnssledp stdyygelqr |
| 301 | disemflqiy kqggflglsn ikfrpgsvvv qltlafregt invhdvetqf nqykteaasr |
| 361 | ynitisdvsv sdvpfpfsaq sgagvpgwgi allvlvcvlv alaivylial avcqcrrkny |
| 421 | gqldifpard tyhpmseypt yhthgryvpp sstdrspyek vsagnggssl sytnpavaat |
| 481 | sanl |

Mucin-1, isoform 11 precursor NP_001191216.1 (SEQ ID NO: 320)

| | |
|---|---|
| 1 | mtpgtqspff llllltvlta ttapkpatvv tgsghasstp ggeketsatq rssvpsstek |
| 61 | nalstgvsff flsfhisnlq fnssledpst dyyqelqrdi semflqiykq ggflglsnik |
| 121 | frpgsvvvql tlafregtin vhdvetqfnq ykteaasryn ltisdvsysd vpfpfsaqsg |
| 181 | agvpgwgial lvlvcvlval aivylialav cqcrrknygq ldifpardty hpmseyptyh |
| 241 | thgryvppss tdrspyekvs agnggsslsy tnpavaatsa nl |

Mucin-1, isoform 12 precursor NP_001191217.1 (SEQ ID NO: 321)

| | |
|---|---|
| 1 | mtpgtqspff llllltvlta ttapkpatvv tgsghasstp ggeketsatq rssvpsstek |
| 61 | nafnssledp stdyyqelqr disemflqiy kqggflglsn ikfrpgsvvv qltlafregt |
| 121 | invhdvetqf nqykteaasr ynltisdvsv wgwgarlghr aagaglcsgc aghclshclg |
| 181 | clsvppkelr aaghlsspgy lpsyervphl phpwalcap |

Mucin-1, isoform 13 precursor NP_001191218.1 (SEQ ID NO: 322)

| | |
|---|---|
| 1 | mtpgtqspff llllltvlta ttapkpatvv tgsghasstp ggeketsatq rssvpsstek |
| 61 | naiykqggfl glsnikfrpg svvvqltlaf regtinvhdv etqfnqykte aasrynitis |
| 121 | dvsysdvpfp fsaqsgagvp gwgiallvlv cvlvalaivy lialavcqcr rknygqldif |
| 181 | pardtyhpms eyptyhthgr yvppsstdrs pyekvsagng gsslsytnpa vaatsanl |

Mucin-1, isoform 14 precursor NP_001191219.1 (SEQ ID NO: 323)

| | |
|---|---|
| 1 | mtpgtqspff llllltvltg geketsatqr ssvpsstekn aiykqggflg lsnikfrpgs |
| 61 | vvvqltlafr egtinvhdve tqfnqyktea asrynitisd vsysdvpfpf saqsgagvpg |
| 121 | wgiallvlvc vlvalaivyl ialavcqcrr knygqldifp ardtyhpmse yptyhthgry |
| 181 | vppsstdrsp yekvsagngg sslsytnpav aatsanl |

Mucin-1, isoform 15 precursor NP_001191220.1 (SEQ ID NO: 324)

| | |
|---|---|
| 1 | mtpgtqspff llllltvlta ttapkpatvv tgsghasstp ggeketsatq rssvpsstek |
| 61 | naflqiykqg gflglsnikf rpgsvvvqlt lafregtinv hdvetqfnqy kteaasrynl |
| 121 | tisdvsysdv pfpfsaqsga gvpgwgiall vlvcvlvala ivylialavc qcrrknygql |
| 181 | difpardtyh pmseyptyht hgryvppsst drspyekvsa gnggsslsyt npavaatsan |
| 241 | l |

Mucin-1, isoform 16 precursor NP_001191221.1 (SEQ ID NO: 325)

| | |
|---|---|
| 1 | mtpgtqspff llllltvlta ttapkpatvv tgsghasstp ggeketsatq rssvpsstek |
| 61 | naipaptttk scretflkwp gsvvvqltla fregtinvhd vetqfnqykt eaasryniti |
| 121 | sdvsysdvpf pfsaqsgagv pgwgiallvl vcvlvalaiv ylialavcqc rrknygqldi |
| 181 | fpardtyhpm seyptyhthg ryvppsstdr spyekvsagn ggsslsytnp avaatsanl |

Mucin-1, isoform 17 precursor NP_001191222.1 (SEQ ID NO: 326)

| | |
|---|---|
| 1 | mtpgtqspff llllltvltv vtgsghasst pggeketsat qrssvpsste knalstgvsf |
| 61 | fflsfhisnl qfnssledps tdyyqelqrd isemflqiyk qggflglsni kfrpgsvvvq |
| 121 | ltlafregti nvhdvetqfn qykteaasry nitisdvsgc lsvppkelra aghlsspgyl |
| 181 | psyervphlp hpwalcap |

Mucin-1, isoform 18 precursor NP_001191223.1 (SEQ ID NO: 327)

| | |
|---|---|
| 1 | mtpgtqspff llllltvltv vtgsghasst pggeketsat qrssvpsste knaipapttt |
| 61 | kscretflkw pgsvvvqltl afregtinvh dvetqfnqyk teaasrynit isdvsysdvp |
| 121 | fpfsaqsgag vpgwgiallv lvcvlvalai vylialavcq crrknygqld ifpardtyhp |
| 181 | mseyptyhth gryvppsstd rspyekvsag nggsslsytn pavaatsanl |

Mucin-1, isoform 19 precursor NP_001191224.1 (SEQ ID NO: 328)

| | |
|---|---|
| 1 | mtpgtqspff llllltvlta ttapkpatvv tgsghasstp ggeketsatq rssvpsstek |
| 61 | nafnssledp stdyyqelqr disemsgagv pgwgiallvl vcvlvalaiv ylialavcqc |
| 121 | rrknygqldi fpardtyhpm seyptyhthg ryvppsstdr spyekvsagn ggsslsytnp |
| 181 | avaatsanl |

Mucin-1, isoform 20 precursor NP_001191225.1 (SEQ ID NO: 329)

| | |
|---|---|
| 1 | mtpgtqspff llllltvlta ttapkpatvv tgsghasstp ggeketsatq rssvpsstek |
| 61 | naipaptttk scretflkcf crfinkgvfw aspilssysd vpfpfsaqsg agvpgwgial |
| 121 | lvlvcvlval aivylialav cqcrrknygq ldifpardty hpmseyptyh thgryvppss |
| 181 | tdrspyekvs agnggsslsy tnpavaatsa nl |

Mucin-1, isoform 21 precursor NP_001191226.1 (SEQ ID NO: 330)

| | |
|---|---|
| 1 | mtpgtqspff llllltvlta ttapkpatvv tgsghasstp ggeketsatq rssvpsstek |
| 61 | nalstgvsff flsfhisnlq fnssledpst dyyqelqrdi semavcqcrr knygqldifp |
| 121 | ardtyhpmse yptyhthgry vppsstdrsp yekvsagngg sslsytnpav aatsanl |

N-myc proto-oncogene protein, isoform 1 NP_001280157.1, NP_005369.2 (SEQ ID
NO: 331)

| | |
|---|---|
| 1 | mpscststmp gmicknpdle fdslqpcfyp deddfyfggp dstppgediw kkfellptpp |
| 61 | lspsrgfaeh sseppswvte mllenelwgs paeedafglg glggltpnpv ilqdcmwsgf |
| 121 | sareklerav seklqhgrgp ptagstaqsp gagaaspagr ghggaagagr agaalpaela |
| 181 | hpaaecvdpa vvfpfpvnkr epapvpaapa sapaagpava sgagiaapag apgvapprpg |
| 241 | grqtsggdhk alstsgedtl sdsddeddee edeeeeidvv tvekrrsssn tkavttftit |
| 301 | vrpknaalgp graqsselil krclpihqqh nyaapspyve sedappqkki kseasprplk |
| 361 | svippkaksl sprnsdseds errrnhnile rqrrndlrss fltlrdhvpe lvknekaakv |
| 421 | vilkkateyv hslqaeehql llekeklqar qqqllkkieh artc |

N-myc proto-oncogene protein, isoform 2 NP_001280160.1 (SEQ ID NO: 332)

| | |
|---|---|
| 1 | mrgapgncvg aeqalarrkr aqtvairghp rppgppgdtr aesppdplqs agddeddeee |
| 61 | deeeeidvvt vekrrsssnt kavttftitv rpknaalgpg raqsselilk rclpihqqhn |
| 121 | yaapspyves edappqkkik seasprplks vippkaksls prnsdsedse rrrnhniler |
| 181 | qrrndlrssf ltlrdhvpel vknekaakvv ilkkateyvh slqaeehqll lekeklqarq |
| 241 | qqllkkieha rtc |

N-myc proto-oncogene protein, isoform 3 NP_001280162.1 (SEQ ID NO: 333)

| | |
|---|---|
| 1 | mrgapgncvg aeqalarrkr aqtvairghp rppgppgdtr aesppdplqs agvlevgagp |
| 61 | rlprppregs tpgiktngae rspqspagrr adaellhvhh aghdlqeprp rv |

Cancer/testis antigen 1B NP_001318.1 (SEQ ID NO: 334)

| | |
|---|---|
| 1 | mqaegrgtgg stgdadgpgg pgipdgpggn aggpgeagat ggrgprgaga arasgpggga |
| 61 | prgphggaas glngccrcga rgpesrllef ylampfatpm eaelarrsla qdapplpvpg |
| 121 | vllkeftvsg niltirltaa dhrqlqlsis sclqqlsllm witqcflpvf laqppsgqrr |

Opioid growth factor receptor NP_031372.2 (SEQ ID NO: 335)

| | |
|---|---|
| 1 | mddpdcdstw eedeedaeda ededcedgea agardadagd edeeseepra arpssfqsrm |
| 61 | tgsrnwratr dmcryrhnyp dlverdcngd tpnlsfyrne irflpngcfi edilqnwtdn |
| 121 | ydllednhsy iqwlfplrep gvnwhakplt lrevevfkss qeiqerlvra yelmlgfygi |
| 181 | rledrgtgtv graqnyqkrf qnlnwrshnn lritrilksl gelglehfqa plvrffleet |
| 241 | lvrrelpgvr qsaldyfmfa vrcrhqrrql vhfawehfrp rckfvwgpqd klrrfkpssl |
| 301 | phplegsrkv eeegspgdpd heastqgrtc gpehskgggr vdegpqprsv epqdagpler |
| 361 | sqgdeagghg edrpeplspk eskkrklels rreqpptepg pqsaseveki alnlegcals |
| 421 | qgslrtgtqe vggqdpgeav qpcrqplgar vadkvrkrrk vdegagdsaa vasggaqtla |
| 481 | lagspapsgh pkaghsengv eedtegrtgp kegtpgspse tpgpspagpa gdepaespse |
| 541 | tpgprpagpa gdepaespse tpgprpagpa gdepaespse tpgpspagpt rdepaespse |
| 601 | tpgprpagpa gdepaespse tpgprpagpa gdepaespse tpgpspagpt rdepakagea |
| 661 | aelqdaeves saksgkp |

P antigen family member 4 NP_001305806.1, NP_008934.1 (SEQ ID NO: 336)

| | |
|---|---|
| 1 | msarvrsrsr grgdgqeapd vvafvapges qqeepptdnq diepgqereg tppieerkve |
| 61 | gdcqemdlek trsergdgsd vkektppnpk haktkeagdg qp |

Paired box protein Pax-3, isoform PAX3a NP_000429.2 (SEQ ID NO: 337)

| | |
|---|---|
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilsergkrwr lgrrtcwvtw rasas |

Paired box protein Pax-3, isoform PAX3i NP_001120838.1 (SEQ ID NO: 338)

| | |
|---|---|
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkvtt pdvekkieey |
| 121 | krenpgmfsw eirdkllkda vcdrntvpsv ssisrilrsk fgkgeeeead lerkeaeese |
| 181 | kkakhsidgi lserasapqs degsdidsep dlplkrkqrr srttftaeql eelerafert |
| 241 | hypdiytree laqrakltea rvqvwfsnrr arwrkqagan qlmafnhlip ggfpptampt |
| 301 | lptyqlsets yqptsipqav sdpsstvhrp qplppstvhq stipsnpdss sayclpstrh |
| 361 | gfssytdsfv ppsgpsnpmn ptignglspq vmglltnhgg vphqpqtdya lspltgglep |
| 421 | tttvsascsq rldhmkslds lptsqsycpp tysttgysmd pvtgyqygqy gqsafhylkp |
| 481 | dia |

Paired box protein Pax-3, isoform PAX3b NP_039230.1 (SEQ ID NO: 339)

| | |
|---|---|
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilsergkalv sgvssh |

Paired box protein Pax-3, isoform PAX3 NP_852122.1 (SEQ ID NO: 340)

| | |
|---|---|
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilserasapq sdegsdidse pdlplkrkqr rsrttftaeq leelerafer |
| 241 | thypdiytre elaqraklte arvqvwfsnr rarwrkqaga nqlmafnhli pggfpptamp |
| 301 | tlptyqlset syqptsipqa vsdpsstvhr pqplppstvh qstipsnpds ssayclpstr |
| 361 | hgfssytdsf vppsgpsnpm nptignglsp qvmglltnhg gvphqpqtdy alspltggle |
| 421 | ptttvsascs qrldhmksld slptsqsycp ptysttgysm dpvtgyqygq ygqskpwtf |

Paired box protein Pax-3, isoform PAX3d NP_852123.1 (SEQ ID NO: 341)

| | |
|---|---|
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilserasapq sdegsdidse pdlplkrkqr rsrttftaeq leelerafer |
| 241 | thypdiytre elaqraklte arvqvwfsnr rarwrkqaga nqlmafnhli pggfpptamp |
| 301 | tlptyqlset syqptsipqa vsdpsstvhr pqplppstvh qstipsnpds ssayclpstr |
| 361 | hgfssytdsf vppsgpsnpm nptignglsp qvmglltnhg gvphqpqtdy alspltggle |
| 421 | ptttvsascs qrldhmksld slptsqsycp ptysttgysm dpvtgyqygq ygqsafhylk |
| 481 | pdia |

Paired box protein Pax-3, isoform PAX3e NP_852124.1 (SEQ ID NO: 342)

| | |
|---|---|
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilserasapq sdegsdidse pdlplkrkqr rsrttftaeq leelerafer |
| 241 | thypdiytre elaqraklte arvqvwfsnr rarwrkqaga nqlmafnhli pggfpptamp |
| 301 | tlptyqlset syqptsipqa vsdpsstvhr pqplppstvh qstipsnpds ssayclpstr |
| 361 | hgfssytdsf vppsgpsnpm nptignglsp qvmglltnhg gvphqpqtdy alspltggle |
| 421 | ptttvsascs qrldhmksld slptsqsycp ptysttgysm dpvtgyqygq ygqsafhylk |
| 481 | pdiawfqill ntfdkssgee edleq |

Paired box protein Pax-3, isoform PAX3h NP_852125.1 (SEQ ID NO: 343)

| | |
|---|---|
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilserasapq sdegsdidse pdlplkrkqr rsrttftaeq leelerafer |
| 241 | thypdiytre elaqraklte arvqvwfsnr rarwrkqaga nqlmafnhli pggfpptamp |
| 301 | tlptyqlset syqptsipqa vsdpsstvhr pqplppstvh qstipsnpds ssayclpstr |
| 361 | hgfssytdsf vppsgpsnpm nptignglsp qvpfiissqi slgfksf |

Paired box protein Pax-3, isoform PAX3g NP_852126.1 (SEQ ID NO: 344)

| | |
|---|---|
| 1 | mttlagavpr mmrpgpgqny prsgfplevs tplgqgrvnq lggvfingrp lpnhirhkiv |
| 61 | emahhgirpc visrqlrvsh gcvskilcry qetgsirpga iggskpkqvt tpdvekkiee |
| 121 | ykrenpgmfs weirdkllkd avcdrntvps vssisrilrs kfgkgeeeea dlerkeaees |
| 181 | ekkakhsidg ilserasapq sdegsdidse pdlplkrkqr rsrttftaeq leelerafer |
| 241 | thypdiytre elaqraklte arvqvwfsnr rarwrkqaga nqlmafnhli pggfpptamp |
| 301 | tlptyqlset syqptsipqa vsdpsstvhr pqplppstvh qstipsnpds ssayclpstr |
| 361 | hgfssytdsf vppsgpsnpm nptignglsp qvpfiissqi srk |

Paired box protein Pax-5, isoform 1 NP_057953.1 (SEQ ID NO: 345)

| | |
|---|---|
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp syssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq tteysamasl agglddmkan lasptpadig ssvpgpqsyp |
| 301 | ivtgrdlast tlpgypphvp pagqgsysap tltgmvpgse fsgspyshpq yssyndswrf |
| 361 | pnpgllgspy yysaaargaa ppaaataydr h |

Paired box protein Pax-5, isoform 2 NP_001267476.1 (SEQ ID NO: 346)

| | |
|---|---|
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp syssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq tteysamasl agglddmkan lasptpadig ssvpgpqsyp |
| 301 | ivtgsefsgs pyshpqyssy ndswrfpnpg llgspyyysa aargaappaa ataydrh |

Paired box protein Pax-5, isoform 3 NP_001267477.1 (SEQ ID NO: 347)

| | |
|---|---|
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp syssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq tteysamasl agglddmkan lasptpadig ssvpgpqsyp |
| 301 | ivtgrdlast tlpgypphvp pagqgsysap tltgmvpgsp yyysaaarga appaaatayd |
| 361 | rh |

Paired box protein Pax-5, isoform 4 NP_001267478.1 (SEQ ID NO: 348)

| | |
|---|---|
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp syssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq gvsfpgvpta tlsiprtttp ggsptrgcla pptiialppe |
| 301 | epphlqpplp mtvtdpwsqa gtkh |

Paired box protein Pax-5, isoform 5 NP_001267479.1 (SEQ ID NO: 349)

| | |
|---|---|
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp syssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq apptiialpp eepphlqppl pmtvtdpwsq agtkh |

Paired box protein Pax-5, isoform 6 NP_001267480.1 (SEQ ID NO: 350)

| | |
|---|---|
| 1 | mfaweirdrl laervcdndt vpsyssinri irtkvqqppn qpvpasshsi vstgsvtqvs |
| 61 | systdsagss ysisgilgit spsadtnkrk rdegiqespv pnghslpgrd flrkqmrgdl |
| 121 | ftqqqlevld rvferqhysd iftttepikp eqtteysama slagglddmk anlasptpad |
| 181 | igssvpgpqs ypivtgspyy ysaaargaap paaataydrh |

Paired box protein Pax-5, isoform 7 NP_001267481.1 (SEQ ID NO: 351)

| | |
|---|---|
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp syssinriir tkvqqppnqp vpasshsivs tgsvtqvssv stdsagssys |
| 181 | isgilgitsp sadtnkrkrd egiqespvpn ghslpgrdfl rkqmrgdlft qqqlevldrv |
| 241 | ferqhysdif tttepikpeq tteysamasl agglddmkan lasptpadig ssvpgpqsyp |
| 301 | ivtgspyyys aaargaappa aataydrh |

Paired box protein Pax-5, isoform 8 NP_001267482.1 (SEQ ID NO: 352)

| | |
|---|---|
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp syssinriir tkvqqppnqp vpasshsigi qespvpnghs lpgrdflrkq |
| 181 | mrgdlftqqq levldrvfer qhysdifttt epikpeqtte ysamaslagg lddmkanlas |
| 241 | ptpadigssv pgpqsypivt grdlasttlp gypphvppag qgsysaptlt gmvpgspyyy |
| 301 | saaargaapp aaataydrh |

Paired box protein Pax-5, isoform 9 NP_001267483.1 (SEQ ID NO: 353)

| | |
|---|---|
| 1 | mdleknyptp rtsrtghggv nqlggvfvng rplpdvvrqr ivelahqgvr pcdisrqlrv |
| 61 | shgcvskilg ryyetgsikp gviggskpkv atpkvvekia eykrqnptmf aweirdrlla |
| 121 | ervcdndtvp syssinriir tkvqqppnqp vpasshsigi qespvpnghs lpgrdflrkq |
| 181 | mrgdlftqqq levldrvfer qhysdifttt epikpeqtte ysamaslagg lddmkanlas |
| 241 | ptpadigssv pgpqsypivt grdlasttlp gypphvppag qgsysaptlt gmvpgsefsg |
| 301 | spyshpqyss yndswrfpnp gllgspyyys aaargaappa aataydrh |

Paired box protein Pax-5, isoform 10 NP_001267484.1 (SEQ ID NO: 354)

| | |
|---|---|
| 1 | mdleknyptp rtsrtghggv nqlggvfvng rplpdvvrqr ivelahqgvr pcdisrqlrv |
| 61 | shgcvskilg riirtkvqqp pnqpvpassh sivstgsvtq vssystdsag ssysisgilg |
| 121 | itspsadtnk rkrdegiqes pvpnghslpg rdflrkqmrg dlftqqqlev ldrvferqhy |
| 181 | sdiftttepi kpeqtteysa maslaggldd mkanlasptp adigssvpgp qsypivtgse |
| 241 | fsgspyshpq yssyndswrf pnpgllgspy yysaaargaa ppaaataydr h |

Paired box protein Pax-5, isoform 11 NP_001267485.1 (SEQ ID NO: 355)

| | |
|---|---|
| 1 | mfaweirdrl laervcdndt vpsyssinri irtkvqqppn qpvpasshsi vstgsvtqvs |
| 61 | systdsagss ysisgilgit spsadtnkrk rdegiqespv pnghslpgrd flrkqmrgdl |
| 121 | ftqqqlevld rvferqhysd iftttepikp eqtteysama slagglddmk anlasptpad |
| 181 | igssvpgpqs ypivtgrdla sttlpgypph vppagqgsys aptltgmvpg sefsgspysh |
| 241 | pqyssyndsw rfpnpgllgs pyyysaaarg aappaaatay drh |

Platelet-derived growth factor receptor beta, isoform 1 NP_002600.1 (SEQ ID
NO: 356)

| | |
|---|---|
| 1 | mrlpgampal alkgelllls lllllepqis qglvvtppgp elvlnvsstf vltcsgsapv |
| 61 | vwermsqepp qemakaqdgt fssvltltnl tgldtgeyfc thndsrglet derkrlyifv |
| 121 | pdptvgflpn daeelfiflt eiteitipcr vtdpqlvvtl hekkgdvalp vpydhqrgfs |
| 181 | gifedrsyic kttigdrevd sdayyvyrlq vssinvsvna vqtvvrqgen itlmcivign |
| 241 | evvnfewtyp rkesgrlvep vtdflldmpy hirsilhips aeledsgtyt cnvtesvndh |
| 301 | qdekainitv vesgyvrllg evgtlqfael hrsrtlqvvf eayppptvlw fkdnrtlgds |
| 361 | sageialstr nvsetryvse ltivrvkvae aghytmrafh edaevqlsfq lqinvpvrvl |
| 421 | elseshpdsg eqtvrcrgrg mpqpniiwsa crdlkrcpre lpptllgnss eeesqletnv |
| 481 | tyweeeqefe vvstlrlqhv drplsvrctl rnavgqdtge vivvphslpf kvvvisaila |
| 541 | lvvltiisli ilimlwqkkp ryeirwkvie syssdgheyi yvdpmqlpyd stwelprdql |
| 601 | vlgrtlgsga fgqvveatah glshsqatmk vavkmlksta rssekqalms elkimshlgp |
| 661 | hlnvvnllga ctkggpiyii teycrygdlv dylhrnkhtf lqhhsdkrrp psaelysnal |
| 721 | pvglplpshv sltgesdggy mdmskdesvd yvpmldmkgd vkyadiessn ymapydnyvp |
| 781 | sapertcrat linespvlsy mdlvgfsyqv angmeflask ncvhrdlaar nvlicegklv |
| 841 | kicdfglard imrdsnyisk gstflplkwm apesifnsly ttlsdvwsfg illweiftlg |
| 901 | gtpypelpmn eqfynaikrg yrmaqpahas deiyeimqkc weekfeirpp fsqlvlller |
| 961 | llgegykkky qqvdeeflrs dhpailrsqa rlpgfhglrs pldtssvlyt avqpnegdnd |
| 1021 | yiiplpdpkp evadegpleg spslasstln evntsstisc dsplepqdep epepqlelqv |
| 1081 | epepeleqlp dsgcpaprae aedsfl |

Platelet-derived growth factor receptor beta, isoform 2 NP_001341945.1 (SEQ
ID NO: 357)

| | |
|---|---|
| 1 | msgeppqema kaqdgtfssv ltltnltgld tgeyfcthnd srgletderk rlyifvpdpt |
| 61 | vgflpndaee lfiflteite itipcrvtdp qlvvtlhekk gdvalpvpyd hqrgfsgife |
| 121 | drsyicktti gdrevdsday yvyrlqvssi nvsvnavqtv vrqgenitlm civignevvn |
| 181 | fewtyprkes grlvepvtdf lldmpyhirs ilhipsaele dsgtytcnvt esvndhqdek |
| 241 | ainitvvesg yvrllgevgt lqfaelhrsr tlqvvfeayp pptvlwfkdn rtlgdssage |
| 301 | ialstrnvse tryvseltiv rvkvaeaghy tmrafhedae vqlsfqlqin vpvrvlelse |
| 361 | shpdsgeqtv rcrgrgmpqp niiwsacrdl krcprelppt llgnsseees qletnvtywe |
| 421 | eeqefevvst lrlqhvdrpl svrctlrnav gqdtqevivv phslpfkvvv isailalvvl |
| 481 | tiisliilim lwqkkpryei rwkviesvss dgheyiyvdp mqlpydstwe lprdqlvlgr |
| 541 | tlgsgafgqv veatahglsh sqatmkvavk mlkstarsse kqalmselki mshlgphlnv |
| 601 | vnllgactkg gpiyiiteyc rygdlvdylh rnkhtflqhh sdkrrppsae lysnalpvgl |
| 661 | plpshvsltg esdggymdms kdesvdyvpm ldmkgdvkya diessnymap ydnyvpsape |
| 721 | rtcratline spvlsymdlv gfsyqvangm eflaskncvh rdlaarnvli cegklvkicd |
| 781 | fglardimrd snyiskgstf lplkwmapes ifnslyttls dvwsfgillw eiftlggtpy |
| 841 | pelpmneqfy naikrgyrma qpahasdeiy eimqkcweek feirppfsql vlllerllge |
| 901 | gykkkyqqvd eeflrsdhpa ilrsqarlpg fhglrspldt ssvlytavqp negdndyiip |
| 961 | lpdpkpevad egplegspsl asstlnevnt sstiscdspl epqdepepep qlelqvepep |
| 1021 | eleqlpdsgc papraeaeds fl |

Platelet-derived growth factor receptor beta, isoform 3 NP_001341946.1 (SEQ
ID NO: 358)

| | |
|---|---|
| 1 | mitnvaflvs lrteatsakp plgtgrwilm ptmstdsrvs plsglmlsrv ssinvsvnav |
| 61 | qtvvrqgeni tlmcivigne vvnfewtypr kesgrlvepv tdflldmpyh irsilhipsa |
| 121 | eledsgtytc nvtesvndhq dekainitvv esgyvrllge vgtlqfaelh rsrtlqvvfe |
| 181 | ayppptvlwf kdnrtlgdss ageialstrn vsetryvsel tlvrvkvaea ghytmrafhe |
| 241 | daevqlsfql qinvpvrvle lseshpdsge qtvrcrgrgm pqpniiwsac rdlkrcprel |
| 301 | pptllgnsse eesqletnvt yweeeqefev vstlrlqhvd rplsvrctlr navgqdtqev |
| 361 | ivvphslpfk vvvisailal vvltiislii limlwqkkpr yeirwkvies vssdgheyiy |
| 421 | vdpmqlpyds twelprdqlv lgrtlgsgaf gqvveatahg lshsqatmkv avkmlkstar |
| 481 | ssekqalmse lkimshlgph lnvvnllgac tkggpiyiit eycrygdlvd ylhrnkhtfl |
| 541 | qhhsdkrrpp saelysnalp vglplpshvs ltgesdggym dmskdesvdy vpmldmkgdv |
| 601 | kyadiessny mapydnyvps apertcratl inespvlsym dlvgfsyqva ngmeflaskn |
| 661 | cvhrdlaarn vlicegklvk icdfglardi mrdsnyiskg stflplkwma pesifnslyt |
| 721 | tlsdvwsfgi llweiftlgg tpypelpmne qfynaikrgy rmaqpahasd eiyeimqkcw |
| 781 | eekfeirppf sqlvlllerl lgegykkkyq qvdeeflrsd hpailrsqar lpgfhglrsp |
| 841 | ldtssvlyta vqpnegdndy iiplpdpkpe vadegplegs pslasstlne vntsstiscd |
| 901 | splepqdepe pepqlelqve pepeleqlpd sgcpapraea edsfl |

Placenta-specific protein 1 precursor NP_001303816.1, NP_001303817.1,
NP_001303818.1, NP_068568.1 (SEQ ID NO: 359)

| | |
|---|---|
| 1 | mkvfkfiglm illtsafsag sgqspmtvlc sidwfmvtvh pfmlnndvcv hfhelhlglg |
| 61 | cppnhvqpha yqftyrvtec girakaysqd mviysteihy sskgtpskfv ipvscaapqk |
| 121 | spwltkpcsm rvasksrata qkdekcyevf slsqssqrpn cdcppcvfse eehtqvpchq |
| 181 | agaqeaqplq pshfldised wslhtddmig sm |

Melanoma antigen preferentially expressed in tumors, isoform a
NP_001278644.1, NP_001278645.1, NP_006106.1, NP_996836.1, NP_996837.1,
NP_996838.1, NP_996839.1 (SEQ ID NO: 360)

| | |
|---|---|
| 1 | merrrlwgsi qsryismsvw tsprrlvela gqsllkdeal aiaalellpr elfpplfmaa |
| 61 | fdgrhsqtlk amvqawpftc lplgvlmkgq hlhletfkav ldgldvllaq evrprrwklq |
| 121 | vldlrknshq dfwtvwsgnr aslysfpepe aaqpmtkkrk vdglsteaeq pfipvevlvd |
| 181 | lflkegacde lfsyliekvk rkknvlrlcc kklkifampm qdikmilkmv qldsiedlev |
| 241 | tctwklptla kfspylgqmi nlrrlllshi hassyispek eeqyiaqfts qflslqclqa |
| 301 | lyvdslfflr grldqllrhv mnpletlsit ncrlsegdvm hlsqspsysq lsvlslsgvm |
| 361 | ltdvspeplq allerasatl qdlvfdecgi tddqllallp slshcsqltt lsfygnsisi |
| 421 | salqsllqhl iglsnlthvl ypvplesyed ihgtlhlerl aylharlrel lcelgrpsmv |
| 481 | wlsanpcphc gdrtfydpep ilcpcfmpn |

Melanoma antigen preferentially expressed in tumors, isoform b
NP_001278646.1, NP_001278648.1, NP_001305055.1, NP_001305056.1 (SEQ ID NO:
361)

| | |
|---|---|
| 1 | msvwtsprrl velagqsllk dealaiaale llprelfppl fmaafdgrhs qtlkamvqaw |
| 61 | pftclplgvl mkgqhlhlet fkavldgldv llaqevrprr wklqvldlrk nshqdfwtvw |
| 121 | sgnraslysf pepeaaqpmt kkrkvdglst eaeqpfipve vlvdlflkeg acdelfsyli |
| 181 | ekvkrkknvl rlcckklkif ampmqdikmi lkmvqldsie dlevtctwkl ptlakfspyl |
| 241 | gqminlrrll lshihassyi spekeeqyia qftsqflslq clqalyvdsl fflrgrldql |
| 301 | lrhvmnplet lsitncrlse gdvmhlsqsp sysqlsvlsl sgvmltdvsp eplqallera |
| 361 | satlqdlvfd ecgitddqll allpslshcs qlttlsfygn sisisalqsl lqhliglsnl |
| 421 | thvlypvple syedihgtlh lerlaylhar lrellcelgr psmvwlsanp cphcgdrtfy |
| 481 | dpepilcpcf mpn |

Phosphatidylinositol 3,4,5-triphosphate-dependent Rac exchanger 2 protein,
isoform a NP_079146.2 (SEQ ID NO: 362)

| | |
|---|---|
| 1 | msedsrgdsr aesakdlekq lrlrvcvlse lqkterdyvg tleflvsafl hrmnqcaask |
| 61 | vdknvteetv kmlfsniedi lavhkeflkv veeclhpepn aqqevgtcfl hfkdkfriyd |
| 121 | eycsnhekaq klllelnkir tirtfllncm llggrkntdv plegylvtpi qrickyplil |
| 181 | kellkrtprk hsdyaavmea lqamkavcsn ineakrqmek levleewqsh iegwegsnit |
| 241 | dtctemlmcg vllkissgni qervfflfdn llvyckrkhr rlknskastd ghrylfrgri |
| 301 | ntevmevenv ddgtadfhss ghivvngwki hntaknkwfv cmaktpeekh ewfeailker |
| 361 | errkglklgm eqdtwvmise qgeklykmmc rqgnlikdrk rklttfpkcf lgsefvswll |
| 421 | eigeihrpee gvhlgqalle ngiihhvtdk hqfkpeqmly rfryddgtfy prnemqdvis |
| 481 | kgvrlycrlh slftpvirdk dyhlrtyksv vmanklidwl iaqgdcrtre eamifgvglc |
| 541 | dngfmhhvle ksefkdepll frffsdeeme gsnmkhrlmk hdlkvvenvi akslliksne |
| 601 | gsygfgledk nkvpiiklve kgsnaemagm evgkkifain gdlvfmrpfn evdcflkscl |
| 661 | nsrkplrvlv stkpretvki pdsadglgfq irgfgpsvvh avgrgtvaaa aglhpgqcii |
| 721 | kvnginvske thasviahvt acrkyrrptk qdsigwvyns iesaqedlqk shskppgdea |
| 781 | gdafdckvee vidkfntmai idgkkehvsl tvdnvhleyg vvyeydstag ikcnvvekmi |
| 841 | epkgffslta kilealaksd ehfvqnctsl nslneviptd lqskfsalcs eriehlcqri |
| 901 | ssykkfsrvl knrawptfkq akskisplhs sdfcptnchv nvmevsypkt stslgsafgv |
| 961 | qldsrkhnsh dkenksseqg klspmvyiqh tittmaapsg lslgqqdghg lryllkeedl |
| 1021 | etqdiyqkll gklqtalkev emcvcqiddl lssityspkl erktsegiip tdsdnekger |
| 1081 | nskrvcfnva gdeqedsghd tisnrdsysd cnsnrnsias ftsicssqcs syfhsdemds |
| 1141 | gdelplsvri shdkqdkihs clehlfsqvd sitnllkgqa vvrafdqtky ltpgrglqef |
| 1201 | qqemepklsc pkrlrlhikq dpwnlpssvr tlaqnirkfv eevkcrllla lleysdsetq |
| 1261 | lrrdmvfcqt lvatvcafse qlmaalnqmf dnskenemet weasrrwldq ianagvlfhf |
| 1321 | qsllspnltd eqamledtlv alfdlekvsf yfkpseeepl vanvpltyqa egsrqalkvy |
| 1381 | fyidsyhfeq lpqrlknggg fkihpvlfaq alesmegyyy rdnvsveefq aqinaaslek |
| 1441 | vkqynqklra fyldksnspp nstskaayvd klmrplnald elyrlvasfi rskrtaacan |
| 1501 | tacsasgvgl lsysselcnr lgachiimcs sgvhrctlsv tleqaiilar shglppryim |
| 1561 | qatdvmrkqg arvqntaknl gvrdrtpqsa prlyklcepp ppagee |

Phosphatidylinositol 3,4,5-triphosphate-dependent Rac exchanger 2 protein,
isoform b NP_079446.3 (SEQ ID NO: 363)

| | |
|---|---|
| 1 | msedsrgdsr aesakdlekq lrlrvcvlse lqkterdyvg tleflvsafl hrmnqcaask |
| 61 | vdknvteetv kmlfsniedi lavhkeflkv veeclhpepn aqqevgtcfl hfkdkfriyd |
| 121 | eycsnhekaq klllelnkir tirtfllncm llggrkntdv plegylvtpi qrickyplil |
| 181 | kellkrtprk hsdyaavmea lqamkavcsn ineakrqmek levleewqsh iegwegsnit |
| 241 | dtctemlmcg vllkissgni qervfflfdn llvyckrkhr rlknskastd ghrylfrgri |
| 301 | ntevmevenv ddgtadfhss ghivvngwki hntaknkwfv cmaktpeekh ewfeailker |
| 361 | errkglklgm eqdtwvmise qgeklykmmc rqgnlikdrk rklttfpkcf lgsefvswll |
| 421 | eigeihrpee gvhlgqalle ngiihhvtdk hqfkpeqmly rfryddgtfy prnemqdvis |
| 481 | kgvrlycrlh slftpvirdk dyhlrtyksv vmanklidwl iaqgdcrtre eamifgvglc |
| 541 | dngfmhhvle ksefkdepll frffsdeeme gsnmkhrlmk hdlkvvenvi akslliksne |
| 601 | gsygfgledk nkvpiiklve kgsnaemagm evgkkifain gdlvfmrpfn evdcflkscl |
| 661 | nsrkplrvlv stkpretvki pdsadglgfq irgfgpsvvh avgrgtvaaa aglhpgqcii |
| 721 | kvnginvske thasviahvt acrkyrrptk qdsigwvyns iesaqedlqk shskppgdea |
| 781 | gdafdckvee vidkfntmai idgkkehvsl tvdnvhleyg vvyeydstag ikcnvvekmi |
| 841 | epkgffslta kilealaksd ehfvqnctsl nslneviptd lqskfsalcs eriehlcqri |
| 901 | ssykkvqase rfynftarha vwehsfdlhs vsstfpvpvt meflllpppl lgisqdgrqh |
| 961 | cipedlpsqe mllaerapv |

Protamine-2, isoform 1 NP_002753.2 (SEQ ID NO: 364)

| | |
|---|---|
| 1 | mvryrvrsls ershevyrqq lhgqeqghhg qeeqglspeh vevyerthgq shyrrrhcsr |
| 61 | rrlhrihrrq hrscrrrkrr scrhrrrhrr gcrtrkrtcr rh |

Protamine-2, isoform 2 NP_001273285.1 (SEQ ID NO: 365)

| | |
|---|---|
| 1 | mvryrvrsls ershevyrqq lhgqeqghhg qeeqglspeh vevyerthgq shyrrrhcsr |
| 61 | rrlhrihrrq hrscrrrkrr scrhrrrhrr eslgdplnqn flsqkaaepg rehaegtklp |
| 121 | gpltpswklr ksrpkhqvrp |

Protamine-2, isoform 3 NP_001273286.1 (SEQ ID NO: 366)

| | |
|---|---|
| 1 | mvryrvrsls ershevyrqq lhgqeqghhg qeeqglspeh vevyerthgq shyrrrhcsr |
| 61 | rrlhrihrrq hrscrrh |

Protamine-2, isoform 4 NP_001273287.1 (SEQ ID NO: 367)

| | |
|---|---|
| 1 | mvryrvrsls ershevyrqq lhgqeqghhg qeeqglspeh vevyerthgq shyrrrhcsr |
| 61 | rrlhrihrrq hrscrrrkrr scrhrrrhrr epgrehaegt klpgpltpsw klrksrpkhq |
| 121 | vrp |

Protamine-2, isoform 5 NP_001273288.1 (SEQ ID NO: 368)

| | |
|---|---|
| 1 | mvryrvrsls ershevyrqq lhgqeqghhg qeeqglspeh vevyerthgq shyrrrhcsr |
| 61 | rrlhrihrrq hrscrrrkrr scrhrrrhrr glpapppcpa cp |

Progranulin NP_002078.1 (SEQ ID NO: 369)

| | |
|---|---|
| 1 | mwtlvswval taglvagtrc pdgqfcpvac cldpggasys ccrplldkwp ttlsrhlggp |
| 61 | cqvdahcsag hsciftvsgt ssccpfpeav acgdghhccp rgfhcsadgr scfqrsgnns |
| 121 | vgaiqcpdsq fecpdfstcc vmvdgswgcc pmpqascced rvhccphgaf cdlvhtrcit |
| 181 | ptgthplakk lpaqrtnrav alsssvmcpd arsrcpdgst ccelpsgkyg ccpmpnatcc |
| 241 | sdhlhccpqd tvcdliqskc lskenattdl ltklpahtvg dvkcdmevsc pdgytccrlq |
| 301 | sgawgccpft qavccedhih ccpagftcdt qkgtceqgph qvpwmekapa hlslpdpqal |
| 361 | krdvpcdnvs scpssdtccq ltsgewgccp ipeavccsdh qhccpqgytc vaegqcqrgs |
| 421 | eivaglekmp arraslshpr digcdqhtsc pvgqtccpsl ggswaccqlp havccedrqh |
| 481 | ccpagytcnv karscekevv saqpatflar sphvgvkdve cgeghfchdn qtccrdnrqg |
| 541 | waccpyrqgv ccadrrhccp agfrcaargt kclrreaprw daplrdpalr qll |

Myeloblastin precursor NP_002768.3 (SEQ ID NO: 370)

| | |
|---|---|
| 1 | mahrppspal asvllallls gaaraaeivg gheaqphsrp ymaslqmrgn pgshfcggtl |
| 61 | ihpsfvltaa hclrdipqrl vnvvlgahnv rtqeptqqhf svaqvflnny daenklndvl |
| 121 | liqlsspanl sasvatvqlp qqdqpvphgt qclamgwgrv gahdppaqvl qelnvtvvtf |
| 181 | fcrphnictf vprrkagicf gdsggplicd giiqgidsfv iwgcatrlfp dfftrvalyv |
| 241 | dwirstlrrv eakgrp |

Prostate stem cell antigen preportein NP_005663.2 (SEQ ID NO: 371)

| | |
|---|---|
| 1 | maglalqpgt allcysckaq vsnedclqve nctqlgeqcw tariravgll tviskgcsln |
| 61 | cvddsqdyyv gkknitccdt dlcnasgaha lqpaaailal lpalglllwg pgql |

Ras-related C3 botulinum toxin substrate 1 isoform Rac1b NP_061485.1 (SEQ ID
NO: 372)

| | |
|---|---|
| 1 | mqaikcvvvg dgavgktcll isyttnafpg eyiptvfdny sanvmvdgkp vnlglwdtag |
| 61 | qedydrlrpl sypqtvgety gkditsrgkd kpiadvflic fslvspasfe nvrakwypev |
| 121 | rhhcpntpii lvgtkldlrd dkdtieklke kkltpitypq glamakeiga vkylecsalt |
| 181 | qrglktvfde airavlcppp vkkrkrkcll l |

Regenerating islet-derived protein 3-alpha precursor NP_002571.1,
NP_620354.1, NP_620355.1 (SEQ ID NO: 373)

| | |
|---|---|
| 1 | mlppmalpsv swmllsclml lsqvqgeepq relpsarirc pkgskaygsh cyalflspks |
| 61 | wtdadlacqk rpsgnlvsvl sgaegsfvss lvksignsys yvwiglhdpt qgtepngegw |
| 121 | ewsssdvmny fawernpsti sspghcasls rstaflrwkd yncnvrlpyv ckftd |

Regulator of G-protein signaling 5, isoform 1 NP_003608.1 (SEQ ID NO: 374)

| | |
|---|---|
| 1 | mckglaalph sclerakeik iklgillqkp dsvgdlvipy nekpekpakt qktsldealq |
| 61 | wrdsldkllq nnyglasfks flksefseen lefwiacedy kkikspakma ekakqiyeef |
| 121 | iqteapkevn idhftkditm knlvepslss fdmaqkriha lmekdslprf vrsefyqeli |
| 181 | k |

Regulator of G-protein signaling 5, isoform 2 NP_001182232.1, NP_001241677.1
(SEQ ID NO: 375)

| | |
|---|---|
| 1 | maekakqiye efiqteapke vnidhftkdi tmknlvepsl ssfdmaqkri halmekdslp |
| 61 | rfvrsefyqe lik |

Regulator of G-protein signaling 5, isoform 3 NP_001241678.1 (SEQ ID NO: 376)

| | |
|---|---|
| 1 | mckglaalph sclerakeik iklgillqkp dsvgdlvipy nekpekpakt qktsldealq |
| 61 | wrdsldkllq nnyglasfks flksefseen lefwiacedy kkikspakma ekakqiyeef |
| 121 | iqteapkevg lwvnidhftk ditmknlvep slssfdmaqk rihalmekds lprfvrsefy |
| 181 | qelik |

Rho-related GTP-binding protein RhoC precursor NP_ 001036143.1,
NP_001036144.1, NP_786886.1 (SEQ ID NO: 377)

| | |
|---|---|
| 1 | maairkklvi vgdgacgktc llivfskdqf pevyvptvfe nyiadievdg kqvelalwdt |
| 61 | agqedydrlr plsypdtdvi lmcfsidspd slenipekwt pevkhfcpnv piilvgnkkd |
| 121 | lrqdehtrre lakmkqepvr seegrdmanr isafgylecs aktkegvrev fematraglq |
| 181 | vrknkrrrgc pil |

Sarcoma antigen 1 NP_061136.2 (SEQ ID NO: 378)

| | |
|---|---|
| 1 | mqasplqtsq ptppeelhaa ayvftndgqq mrsdevnlva tghqskkkhs rkskrhsssk |
| 61 | rrksmsswld kqedaavths iceerinngq pvadnvlsta ppwpdatiah nireermeng |
| 121 | qsrtdkvlst appqlvhmaa agipsmstrd lhstvthnir eermengqpq pdnvlstgpt |
| 181 | glinmaatpi pamsardlya tvthnvceqk menvqpapdn vlltlrprri nmtdtgispm |
| 241 | strdpyatit ynvpeekmek gqpqpdnils tastglinva gagtpaistn glystvphnv |
| 301 | ceekmendqp qpnnvlstvq pviiyltatg ipgmntrdqy atithnvcee rvvnnqplps |
| 361 | nalstvlpgl aylatadmpa mstrdqhati ihnlreekkd nsqptpdnvl savtpelinl |
| 421 | agagippmst rdqyatvnhh vhearmengq rkqdnvlsnv lsglinmaga sipamssrdl |
| 481 | yatithsvre ekmesgkpqt dkvisndapq lghmaaggip smstkdlyat vtqnvheerm |
| 541 | ennqpqpsyd lstvlpglty ltvagipams trdqyatvth nvheekikng qaasdnvfst |
| 601 | vppafinmaa tgvssmstrd qyaavthnir eekinnsqpa pgnilstapp wlrhmaaagi |
| 661 | sstitrdlyv tathsvheek mtngqqapdn slstvppgci nlsgagiscr strdlyatvi |
| 721 | hdiqeeemen dqtppdgfls nsdspelinm tghcmppnal dsfshdftsl skdellykpd |
| 781 | snefavgtkn ysysagdppv tvmslvetvp ntpqispama kkinddikyq lmkevrrfgq |
| 841 | nyerifille evqgsmkvkr qfveftikea arfkkvvliq qlekalkeid shchlrkvkh |
| 901 | mrkr |

Squamous cell carcinoma antigen recognized by T-cells 3 NP_055521.1 (SEQ ID
NO: 379)

| | |
|---|---|
| 1 | mataaetsas epeaeskagp kadgeedevk aartrrkvls ravaaatykt mgpawdqqee |
| 61 | gvsesdgdey amassaessp geyeweydee eeknqleier leeqlsinvy dynchvdlir |
| 121 | llrlegeltk vrmarqkmse ifplteelwl ewlhdeisma qdgldrehvy dlfekavkdy |
| 181 | icpniwleyg qysvggigqk gglekvrsvf eralssvglh mtkglalwea yrefesaive |
| 241 | aarlekvhsl frrqlaiply dmeatfaeye ewsedpipes viqnynkalq qlekykpyee |
| 301 | allqaeaprl aeyqayidfe mkigdpariq liferalven clvpdlwiry sqyldrqlkv |
| 361 | kdlvlsvhnr airncpwtva lwsryllame rhgvdhqvis vtfekalnag fiqatdyvei |
| 421 | wqayldylrr rvdfkqdssk eleelraaft raleylkqev eerfnesgdp scvimqnwar |
| 481 | iearlcnnmq karelwdsim trgnakyanm wleyynlera hgdtqhcrka lhravqctsd |
| 541 | ypehvcevll tmertegsle dwdiavqkte trlarvneqr mkaaekeaal vqqeeekaeq |
| 601 | rkraraekka lkkkkkirgp ekrgadedde kewgddeeeq pskrrrvens ipaagetqnv |
| 661 | evaagpagkc aavdveppsk qkekaaslkr dmpkvlhdss kdsitvfvsn lpysmqepdt |
| 721 | klrplfeacg evvqirpifs nrgdfrgycy vefkeeksal qalemdrksv egrpmfvspc |
| 781 | vdksknpdfk vfrystslek hklfisglpf sctkeeleei ckahgtvkdl rlvtnragkp |
| 841 | kglayveyen esqasqavmk mdgmtikeni ikvaisnppq rkvpekpetr kapggpmllp |
| 901 | qtygargkgr tqlsllpral qrpsaaapqa engpaaapav aapaateapk msnadfaklf |
| 961 | lrk |

Secretory leukocyte protein inhibitor NP_003055.1 (SEQ ID NO: 380)

| | |
|---|---|
| 1 | mkssglfpfl vllalgtlap wavegsgksf kagvcppkks aqclrykkpe cqsdwqcpgk |
| 61 | krccpdtcgi kcldpvdtpn ptrrkpgkcp vtygqclmln ppnfcemdgq ckrdlkccmg |
| 121 | mcgkscvspv ka |

Transcription factor SOX-10 NP_008872.1 (SEQ ID NO: 381)

| | |
|---|---|
| 1 | maeeqdlsev elspvgseep rclspgsaps lgpdgggggs glraspgpge lgkvkkeqqd |
| 61 | geadddkfpv cireaysqvl sgydwtivpm pvrvngasks kphvkrpmna fmvwaqaarr |
| 121 | kladqyphlh naelsktlgk lwrllnesdk rpfieeaerl rmqhkkdhpd ykyqprrrkn |
| 181 | gkaaqgeaec pggeaeqggt aaiqahyksa hldhrhpgeg spmsdgnpeh psgqshgppt |
| 241 | ppttpktelq sgkadpkrdg rsmgeggkph idfgnvdige ishevmsnme tfdvaeldqy |
| 301 | lppnghpghv ssysaagygl gsalavasgh sawiskppgv alptvsppgv dakaqvktet |
| 361 | agpqgpphyt dqpstsqiay tslslphygs afpsisrpqf dysdhqpsgp yyghsgqasg |
| 421 | lysafsymgp sqrplytais dpspsgpqsh spthweqpvy ttlsrp |

Sperm surface protein Sp17 NP_059121.1 (SEQ ID NO: 382)

| | |
|---|---|
| 1 | msipfsnthy ripqgfgnll egltreilre qpdnipafaa ayfesllekr ektnfdpaew |
| 61 | gskvedrfyn nhafeeqepp eksdpkqees qisgkeeets vtildsseed kekeevaavk |
| 121 | iqaafrghia reeakkmktn slqneekeen k |

Protein SSX2, isoform a NP_003138.3 (SEQ ID NO: 383)

| | |
|---|---|
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgnrea qekeerrgta hrwssqnthn |
| 181 | igrfslstsm gavhgtpkti thnrdpkggn mpgptdcvre nsw |

Protein SSX2, isoform b NP_783629.1 (SEQ ID NO: 384)

| | |
|---|---|
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgpkrg ehawthrlre rkqlviyeei |
| 181 | sdpeedde |

Protein SSX2, isoform c NP_001265626.1 (SEQ ID NO: 385)

| | |
|---|---|
| 1 | mngddafarr ptvgaqipek iqkafddiak yfskeewekm kasekifyvy mkrkyeamtk |
| 61 | lgfkatlppf mcnkraedfq gndldndpnr gnqverpqmt fgrlqgispk impkkpaeeg |
| 121 | ndseevpeas gpqndgkelc ppgkpttsek ihersgnrea qekeerrgta hrwssqnthn |
| 181 | igpkrgehaw thrlrerkql viyeeisdpe edde |

Lactosylceramide alpha-2,3-sialyltransferase, isoform 1 NP_003887.3 (SEQ ID
NO: 386)

| | |
|---|---|
| 1 | mrtkaagcae rrplqprtea aaapagramp seytyvklrs dcsrpslqwy traqskmrrp |
| 61 | slllkdilkc tllvfgvwil yilklnytte ecdmkkmhyv dpdhvkraqk yaqqvlqkec |
| 121 | rpkfaktsma llfehrysvd llpfvqkapk dseaeskydp pfgfrkfssk vqtllellpe |
| 181 | hdlpehlkak tcrrcvvigs ggilhglelg htlnqfdvvi rlnsapvegy sehvgnktti |
| 241 | rmtypegapl sdleyysndl fvavlfksvd fnwlqamvkk etlpfwvrlf fwkqvaekip |
| 301 | lqpkhfriln pviiketafd ilqysepqsr fwgrdknvpt igviavvlat hlcdevslag |
| 361 | fgydlnqprt plhyfdsqcm aamnfqtmhn vttetkfllk lvkegvvkdl sggidref |

Lactosylceramide alpha-2,3-sialyltransferase, isoform 2 NP_001035902.1 (SEQ
ID NO: 387)

| | |
|---|---|
| 1 | masvpmpsey tyvklrsdcs rpslqwytra qskmrrpsll lkdilkctll vfgvwilyil |
| 61 | klnytteecd mkkmhyvdpd hvkraqkyaq qvlqkecrpk faktsmallf ehrysvdllp |
| 121 | fvqkapkdse aeskydppfg frkfsskvqt llellpehdl pehlkaktcr rcvvigsggi |
| 181 | lhglelghtl nqfdvvirln sapvegyseh vgnkttirmt ypegaplsdl eyysndlfva |
| 241 | vlfksvdfnw lqamvkketl pfwvrlffwk qvaekiplqp khfrilnpvi iketafdilq |
| 301 | ysepqsrfwg rdknvptigv iavvlathlc devslagfgy dlnqprtplh yfdsqcmaam |
| 361 | nfqtmhnvtt etkfllklvk egvvkdlsgg idref |

Lactosylceramide alpha-2,3-sialyltransferase, isoform 3 NP_001341152.1,
NP_001341153.1, NP_001341155.1, NP_001341162.1, NP_001341163.1,
NP_001341177.1 (SEQ ID NO: 388)

| | |
|---|---|
| 1 | mallfehrys vdllpfvqka pkdseaesky dppfgfrkfs skvqtllell pehdlpehlk |
| 61 | aktcrrcvvi gsggilhgle lghtlnqfdv virlnsapve gysehvgnkt tirmtypega |
| 121 | plsdleyysn dlfvavlfks vdfnwlqamv kketlpfwvr lffwkqvaek iplqpkhfri |
| 181 | lnpviiketa fdilqysepq srfwgrdknv ptigviavvl athlcdevsl agfgydlnqp |
| 241 | rtplhyfdsq cmaamnfqtm hnvttetkfl lklvkegvvk dlsggidref |

Lactosylceramide alpha-2,3-sialyltransferase, isoform 4 NP_001341156.1,
NP_001341158.1, NP_001341167.1 (SEQ ID NO: 389)

| | |
|---|---|
| 1 | mpseytyvkl rsdcsrpslq wytraqskmr rpslllkdil kctllvfgvw ilyilklnyt |
| 61 | teecdmkkmh yvdpdhvkra qkyaqqvlqk ecrpkfakts mallfehrys vdllpfvqka |
| 121 | pkdseaesky dppfgfrkfs skvqtllell pehdlpehlk aktcrrcvvi gsggilhgle |
| 181 | lghtlnqfdv virinsapve gysehvgnkt tirmtypega plsdleyysn dlfvavlfks |
| 241 | vdfnwlqamv kketlpfwvr lffwkqvaek iplqpkhfri lnpviiketa fdilqysepq |
| 301 | srfwgrdknv ptigviavvl athlcdevsl agfgydlnqp rtplhyfdsq cmaamnfqtm |
| 361 | hnvttetkfl lklvkegvvk dlsggidref |

Lactosylceramide alpha-2,3-sialyltransferase, isoform 5 NP_001341176.1 (SEQ
ID NO: 390)

| | |
|---|---|
| 1 | mtypegapls dleyysndlf vavlfksvdf nwlqamvkke tlpfwvrlff wkqvaekipl |
| 61 | qpkhfrilnp viiketafdi lqysepqsrf wgrdknvpti gviavvlath lcdevslagf |
| 121 | gydlnqprtp lhyfdsqcma amnfqtmhnv ttetkfllkl vkegvvkdls ggidref |

Alpha-N-acetylneuraminide alpha-2,8-sialyltransferase, isoform 1 NP_003025.1
(SEQ ID NO: 391)

| | |
|---|---|
| 1 | mspcgrarrq tsrgamavla wkfprtrlpm gasalcvvvl cwlyifpvyr lpnekeivqg |
| 61 | vlqqgtawrr nqtaarafrk qmedccdpah lfamtkmnsp mgksmwydge flysftidns |
| 121 | tyslfpqatp fqlplkkcav vgnggilkks gcgrqidean fvmrcnlppl sseytkdvgs |
| 181 | ksqlvtanps iirqrfqnll wsrktfvdnm kiynhsyiym pafsmktgte pslrvyytls |
| 241 | dvganqtvlf anpnflrsig kfwksrgiha krlstglflv saalglceev aiygfwpfsv |
| 301 | nmheqpishh yydnvlpfsg fhampeeflq lwylhkigal rmqldpcedt slqpts |

Alpha-N-acetylneuraminide alpha-2,8-sialyltransferase, isoform 2
NP_001291379.1 (SEQ ID NO: 392)

| | |
|---|---|
| 1 | mtgsfythsp ltiqltlssh rcnlpplsse ytkdvgsksq lvtanpsiir qrfqnllwsr |
| 61 | ktfvdnmkiy nhsyiympaf smktgtepsl rvyytlsdvg anqtvlfanp nflrsigkfw |
| 121 | ksrgihakrl stglflvsaa lglceevaiy gfwpfsvnmh eqpishhyyd nvlpfsgfha |
| 181 | mpeeflqlwy lhkigalrmq ldpcedtslq pts |

Survivin, isoform 1 NP_001159.2 (SEQ ID NO: 393)

| | |
|---|---|
| 1 | mgaptlppaw qpflkdhris tfknwpfleg cactpermae agfihcpten epdlaqcffc |
| 61 | fkelegwepd ddpieehkkh ssgcaflsvk kqfeeltlge flkldrerak nkiaketnnk |
| 121 | kkefeetaek vrraieqlaa md |

Survivin, isoform 2 NP_001012270.1 (SEQ ID NO: 394)

| | |
|---|---|
| 1 | mgaptlppaw qpflkdhris tfknwpfleg cactpermae agfihcpten epdlaqcffc |
| 61 | fkelegwepd ddpmqrkpti rrknlrklrr kcavpssswl pwieasgrsc lvpewlhhfq |
| 121 | glfpgatslp vgplams |

Survivin, isoform 3 NP_001012271.1 (SEQ ID NO: 395)

| | |
|---|---|
| 1 | mgaptlppaw qpflkdhris tfknwpfleg cactpermae agfihcpten epdlaqcffc |
| 61 | fkelegwepd ddpigpgtva yacntstlgg rggritreeh kkhssgcafl svkkqfeelt |
| 121 | lgeflkldre raknkiaket nnkkkefeet aekvrraieq laamd |

T-box 4, isoform 1 NP_001308049.1 (SEQ ID NO: 396)

| | |
|---|---|
| 1 | mlqdkglses eeafrapgpa lgeasaanap epalaapgls gaalgsppgp gadvvaaaaa |
| 61 | eqtienikvg lhekelwkkf heagtemiit kagrrmfpsy kvkvtgmnpk tkyillidiv |
| 121 | paddhrykfc dnkwmvagka epampgrlyv hpdspatgah wmrqlvsfqk lkltnnhldp |
| 181 | fghiilnsmh kyqprlhivk adennafgsk ntafcthvfp etsfisvtsy qnhkitqlki |
| 241 | ennpfakgfr gsddsdlrva rlqskeypvi sksimrqrli spqlsatpdv gpllgthqal |
| 301 | qhyqhengah sqlaepqdlp lstfptqrds slfyhclkrr adgtrhldlp ckrsyleaps |
| 361 | svgedhyfrs pppydqqmls psycsevtpr eacmysgsgp eiagvsgvdd lpppplscnm |
| 421 | wtsyspytsy svqtmetvpy qpfpthftat tmmprlptls aqssqppgna hfsvynqlsq |
| 481 | sqvrergpsa sfprerglpq gcerkppsph lnaaneflys qtfslsress lqyhsgmgtv |
| 541 | enwtdg |

T-box 4, isoform 2 NP_ 060958.2 (SEQ ID NO: 397)

| | |
|---|---|
| 1 | mlqdkglses eeafrapgpa lgeasaanap epalaapgls gaalgsppgp gadvvaaaaa |
| 61 | eqtienikvg lhekelwkkf heagtemiit kagrrmfpsy kvkvtgmnpk tkyillidiv |
| 121 | paddhrykfc dnkwmvagka epampgrlyv hpdspatgah wmrqlvsfqk lkltnnhldp |
| 181 | fghiilnsmh kyqprlhivk adennafgsk ntafcthvfp etsfisvtsy qnhkitqlki |
| 241 | ennpfakgfr gsddsdlrva rlqskeypvi sksimrqrli spqlsatpdv gpllgthqal |
| 301 | qhyqhengah sqlaepqdlp lstfptqrds slfyhclkrr dgtrhldlpc krsyleapss |
| 361 | vgedhyfrsp ppydqqmlsp sycsevtpre acmysgsgpe iagvsgvddl pppplscnmw |
| 421 | tsyspytsys vqtmetvpyq pfpthftatt mmprlptlsa qssqppgnah fsvynqlsqs |
| 481 | qvrergpsas fprerglpqg cerkppsphl naaneflysq tfslsressl qyhsgmgtve |
| 541 | nwtdg |

Angiopoietin-1 receptor, isoform 1 NP_000450.2 (SEQ ID NO: 398)

| | |
|---|---|
| 1 | mdslaslvlc gvslllsgtv egamdlilin slplvsdaet sltciasgwr phepitigrd |
| 61 | fealmnqhqd plevtqdvtr ewakkvvwkr ekaskingay fcegrvrgea irirtmkmrq |
| 121 | qasflpatlt mtvdkgdnvn isfkkvlike edaviykngs fihsvprhev pdilevhlph |
| 181 | aqpqdagvys aryiggnlft saftrlivrr ceaqkwgpec nhlctacmnn gvchedtgec |
| 241 | icppgfmgrt cekacelhtf grtckercsg qegcksyvfc lpdpygcsca tgwkglqcne |
| 301 | achpgfygpd cklrcscnng emcdrfqgcl cspgwqglqc eregiprmtp kivdlpdhie |
| 361 | vnsgkfnpic kasgwplptn eemtlvkpdg tvlhpkdfnh tdhfsvaift ihrilppdsg |
| 421 | vwvcsvntva gmvekpfnis vkvlpkpina pnvidtghnf avinissepy fgdgpikskk |
| 481 | llykpvnhye awqhiqvtne ivtlnylepr teyelcvqlv rrgeggeghp gpvrrfttas |
| 541 | iglppprgln llpksqttln ltwqpifpss eddfyvever rsvqksdqqn ikvpgnitsv |
| 601 | llnnlhpreq yvvrarvntk aqgewsedlt awtlsdilpp qpenikisni thssaviswt |
| 661 | ildgysissi tirykvqgkn edqhvdvkik natitqyqlk glepetayqv difaennigs |
| 721 | snpafshelv tlpesqapad lgggkmllia ilgsagmtcl tvllafliil qlkranvqrr |
| 781 | maqafqnvre epavqfnsgt lalnrkvknn pdptiypvld wndikfqdvi gegnfgqvlk |
| 841 | arikkdglrm daaikrmkey askddhrdfa gelevlcklg hhpniinllg acehrgylyl |
| 901 | aieyaphgnl ldflrksrvl etdpafaian stastlssqq llhfaadvar gmdylsqkqf |
| 961 | ihrdlaarni lvgenyvaki adfglsrgqe vyvkktmgrl pvrwmaiesl nysvyttnsd |
| 1021 | vwsygvllwe ivslggtpyc gmtcaelyek lpqgyrlekp lncddevydl mrqcwrekpy |
| 1081 | erpsfaqilv slnrmleerk tyvnttlyek ftyagidcsa eeaa |

Angiopoietin-1 receptor, isoform 2 NP_001277006.1 (SEQ ID NO: 399)

| | |
|---|---|
| 1 | mdslaslvlc gvslllsgtv egamdlilin slplvsdaet sltciasgwr phepitigrd |
| 61 | fealmnqhqd plevtqdvtr ewakkvvwkr ekaskingay fcegrvrgea irirtmkmrq |
| 121 | qasflpatlt mtvdkgdnvn isfkkvlike edaviykngs fihsvprhev pdilevhlph |
| 181 | aqpqdagvys aryiggnlft saftrlivrr ceaqkwgpec nhlctacmnn gvchedtgec |
| 241 | icppgfmgrt cekacelhtf grtckercsg qegcksyvfc lpdpygcsca tgwkglqcne |
| 301 | giprmtpkiv dlpdhievns gkfnpickas gwplptneem tlvkpdgtvl hpkdfnhtdh |
| 361 | fsvaiftihr ilppdsgvwv csvntvagmv ekpfnisvkv lpkpinapnv idtghnfavi |
| 421 | nissepyfgd gpikskklly kpvnhyeawq hiqvtneivt lnyleprtey elcvqlvrrg |
| 481 | eggeghpgpv rrfttasigl ppprglnllp ksqttlnltw qpifpssedd fyveverrsv |
| 541 | qksdqqnikv pgnitsvlln nlhpreqyvv rarvntkaqg ewsedltawt lsdilppqpe |
| 601 | nikisniths saviswtild gysissitir ykvqgknedq hvdvkiknat itqyqlkgle |
| 661 | petayqvdif aennigssnp afshelvtlp esqapadlgg gkmlliailg sagmtcltvl |
| 721 | lafliilqlk ranvqrrmaq afqnvreepa vqfnsgtlal nrkvknnpdp tiypvldwnd |
| 781 | ikfqdvigeg nfgqvlkari kkdglrmdaa ikrmkeyask ddhrdfagel evlcklghhp |
| 841 | niinllgace hrgylylaie yaphgnlldf lrksrvletd pafaiansta stlssqqllh |
| 901 | faadvargmd ylsqkqfihr dlaarnilvg enyvakiadf glsrgqevyv kktmgrlpvr |
| 961 | wmaieslnys vyttnsdvws ygvllweivs lggtpycgmt caelyeklpq gyrlekplnc |
| 1021 | ddevydlmrq cwrekpyerp sfaqilvsln rmleerktyv nttlyekfty agidcsaeea |
| 1081 | a |

Angiopoietin-1 receptor, isoform 3 NP_001277007.1 (SEQ ID NO: 400)

| | |
|---|---|
| 1 | mdslaslvlc gvslllsasf lpatltmtvd kgdnvnisfk kvlikeedav iykngsfihs |
| 61 | vprhevpdil evhlphaqpq dagvysaryi ggnlftsaft rlivrrceaq kwgpecnhlc |
| 121 | tacmnngvch edtgecicpp gfmgrtceka celhtfgrtc kercsgqegc ksyvfclpdp |
| 181 | ygcscatgwk glqcnegipr mtpkivdlpd hievnsgkfn pickasgwpl ptneemtivk |
| 241 | pdgtvlhpkd fnhtdhfsva iftihrilpp dsgvwvcsvn tvagmvekpf nisvkvlpkp |
| 301 | lnapnvidtg hnfaviniss epyfgdgpik skkllykpvn hyeawqhiqv tneivtlnyl |
| 361 | eprteyelcv qlvrrgegge ghpgpvrrft tasiglpppr glnllpksqt tlnitwqpif |
| 421 | psseddfyve verrsvqksd qgnikvpgnl tsvllnnlhp reqyvvrarv ntkaqgewse |
| 481 | dltawtlsdi lppqpeniki snithssavi swtildgysi ssitirykvq gknedqhvdv |
| 541 | kiknatitqy qlkglepeta yqvdifaenn igssnpafsh elvtlpesqa padlgggkml |
| 601 | liailgsagm tcltvllafl iilqlkranv qrrmaqafqn reepavqfns gtlalnrkvk |
| 661 | nnpdptiypv ldwndikfqd vigegnfgqv lkarikkdgl rmdaaikrmk eyaskddhrd |
| 721 | fagelevlck lghhpniinl lgacehrgyl ylaieyaphg nlldflrksr vletdpafai |
| 781 | anstastlss qqllhfaadv argmdylsqk qfihrdlaar nilvgenyva kiadfglsrg |
| 841 | qevyvkktmg rlpvrwmaie slnysvyttn sdvwsygvll weivslggtp ycgmtcaely |
| 901 | eklpqgyrle kplncddevy dlmrqcwrek pyerpsfaqi lvslnrmlee rktyvnttly |
| 961 | ekftyagidc saeeaa |

Telomerase reverse transcriptase, isoform 1 NP_937983.2 (SEQ ID NO: 401)

| | |
|---|---|
| 1 | mpraprcrav rsllrshyre vlplatfvrr lgpqgwrlvq rgdpaafral vaqclvcvpw |
| 61 | darpppaaps frqvsclkel varvlqrlce rgaknvlafg falldgargg ppeafttsvr |
| 121 | sylpntvtda lrgsgawgll lrrvgddvlv hllarcalfv lvapscayqv cgpplyqlga |
| 181 | atqarpppha sgprrrlgce rawnhsvrea gvplglpapg arrrggsasr slplpkrprr |
| 241 | gaapepertp vgqgswahpg rtrgpsdrgf cvvsparpae eatslegals gtrhshpsvg |
| 301 | rqhhagppst srpprpwdtp cppvyaetkh flyssgdkeq lrpsfllssl rpsltgarrl |
| 361 | vetiflgsrp wmpgtprrlp rlpqrywqmr plflellgnh aqcpygvllk thcplraavt |
| 421 | paagvcarek pqgsvaapee edtdprrlvq llrqhsspwq vygfvraclr rlvppglwgs |
| 481 | rhnerrflrn tkkfislgkh aklslqeltw kmsvrdcawl rrspgvgcvp aaehrlreei |
| 541 | lakflhwlms vyvvellrsf fyvtettfqk nrlffyrksv wsklqsigir qhlkrvqlre |
| 601 | lseaevrqhr earpalltsr lrfipkpdgl rpivnmdyvv gartfrrekr aerltsrvka |
| 661 | lfsvinyera rrpgllgasv lglddihraw rtfvlrvraq dpppelyfvk vdvtgaydti |
| 721 | pqdrltevia siikpqntyc vrryavvqka ahghvrkafk shvstltdlq pymrqfvahl |
| 781 | qetsplrdav vieqssslne assglfdvfl rfmchhavri rgksyvqcqg ipqgsilstl |
| 841 | lcslcygdme nklfagirrd glllrlvddf llvtphltha ktflrtivrg vpeygcvvnl |
| 901 | rktvvnfpve dealggtafv qmpahglfpw cgllldtrtl evqsdyssya rtsirasltf |
| 961 | nrgfkagrnm rrklfgvlrl kchslfldlq vnslqtvctn iykilllqay rfhacvlqlp |
| 1021 | fhqqvwknpt fflrvisdta slcysilkak nagmslgakg aagplpseav qwlchqafll |
| 1081 | kltrhrvtyv pllgslrtaq tqlsrklpgt tltaleaaan palpsdfkti ld |

Telomerase reverse transcriptase, isoform 2 NP_001180305.1 (SEQ ID NO: 402)

| | |
|---|---|
| 1 | mpraprcrav rsllrshyre vlplatfvrr lgpqgwrlvq rgdpaafral vaqclvcvpw |
| 61 | darpppaaps frqvsclkel varvlqrlce rgaknvlafg falldgargg ppeafttsvr |
| 121 | sylpntvtda lrgsgawgll lrrvgddvlv hllarcalfv lvapscayqv cgpplyqlga |
| 181 | atqarpppha sgprrrlgce rawnhsvrea gvplglpapg arrrggsasr slplpkrprr |
| 241 | gaapepertp vgqgswahpg rtrgpsdrgf cvvsparpae eatslegals gtrhshpsvg |
| 301 | rqhhagppst srpprpwdtp cppvyaetkh flyssgdkeq lrpsfllssl rpsltgarrl |
| 361 | vetiflgsrp wmpgtprrlp rlpqrywqmr plflellgnh aqcpygvllk thcplraavt |
| 421 | paagvcarek pqgsvaapee edtdprrlvq llrghsspwq vygfvraclr rlvppglwgs |
| 481 | rhnerrflrn tkkfislgkh aklslqeltw kmsvrdcawl rrspgvgcvp aaehrlreei |
| 541 | lakflhwlms vyvvellrsf fyvtettfqk nrlffyrksv wsklqsigir qhlkrvqlre |
| 601 | lseaevrqhr earpalltsr lrfipkpdgl rpivnmdyvv gartfrrekr aerltsrvka |
| 661 | lfsvinyera rrpgllgasv lglddihraw rtfvlrvraq dpppelyfvk vdvtgaydti |
| 721 | pqdrltevia siikpqntyc vrryavvqka ahghvrkafk shvstltdlq pymrqfvahl |
| 781 | qetsplrdav vieqssslne assglfdvfl rfmchhavri rgksyvqcqg ipqgsilstl |
| 841 | lcslcygdme nklfagirrd glllrlvddf llvtphltha ktflsyarts irasltfnrg |
| 901 | fkagrnmrrk lfgvlrlkch slfldlqvns lqtvctniyk illlqayrfh acvlqlpfhq |
| 961 | qvwknptffl rvisdtaslc ysilkaknag mslgakgaag plpseavqwl chqafllklt |
| 1021 | rhrvtyvpll gslrtaqtql srklpgttlt aleaaanpal psdfktild |

Cellular tumor antigen p53, isoform a NP_000537.3, NP_001119584.1 (SEQ ID NO:
403)

| | |
|---|---|
| 1 | meepqsdpsv epplsqetfs dlwkllpenn vlsplpsqam ddlmlspddi eqwftedpgp |
| 61 | deaprmpeaa ppvapapaap tpaapapaps wplsssvpsq ktyqgsygfr lgflhsgtak |
| 121 | svtctyspal nkmfcqlakt cpvqlwvdst pppgtrvram aiykqsqhmt evvrrcphhe |
| 181 | rcsdsdglap pqhlirvegn lrveylddrn tfrhsvvvpy eppevgsdct tihynymcns |
| 241 | scmggmnrrp iltiitleds sgnllgrnsf evrvcacpgr drrteeenlr kkgephhelp |
| 301 | pgstkralpn ntssspqpkk kpldgeyftl qirgrerfem frelnealel kdaqagkepg |
| 361 | gsrahsshlk skkgqstsrh kklmfktegp dsd |

Cellular tumor antigen p53, isoform b NP_001119586.1 (SEQ ID NO: 404)

| | |
|---|---|
| 1 | meepqsdpsv epplsqetfs dlwkllpenn vlsplpsqam ddlmlspddi eqwftedpgp |
| 61 | deaprmpeaa ppvapapaap tpaapapaps wplsssvpsq ktyqgsygfr lgflhsgtak |
| 121 | svtctyspal nkmfcqlakt cpvqlwvdst pppgtrvram aiykqsqhmt evvrrcphhe |
| 181 | rcsdsdglap pqhlirvegn lrveylddrn tfrhsvvvpy eppevgsdct tihynymcns |
| 241 | scmggmnrrp iltiitleds sgnllgrnsf evrvcacpgr drrteeenlr kkgephhelp |
| 301 | pgstkralpn ntssspqpkk kpldgeyftl qdqtsfqken c |

Cellular tumor antigen p53, isoform c NP_001119585.1 (SEQ ID NO: 405)

| | |
|---|---|
| 1 | meepqsdpsv epplsqetfs dlwkllpenn vlsplpsqam ddlmlspddi eqwftedpgp |
| 61 | deaprmpeaa ppvapapaap tpaapapaps wplsssvpsq ktyqgsygfr lgflhsgtak |
| 121 | svtctyspal nkmfcqlakt cpvqlwvdst pppgtrvram aiykqsqhmt evvrrcphhe |
| 181 | rcsdsdglap pqhlirvegn lrveylddrn tfrhsvvvpy eppevgsdct tihynymcns |
| 241 | scmggmnrrp iltiitleds sgnllgrnsf evrvcacpgr drrteeenlr kkgephhelp |
| 301 | pgstkralpn ntssspqpkk kpldgeyftl qmlldkrwcy flinss |

Cellular tumor antigen p53, isoform d NP_001119587.1 (SEQ ID NO: 406)

| | |
|---|---|
| 1 | mfcqlaktcp vqlwvdstpp pgtrvramai ykqsqhmtev vrrcphherc sdsdglappq |
| 61 | hlirvegnlr veylddrntf rhsvvvpyep pevgsdctti hynymcnssc mggmnrrpil |
| 121 | tiitledssg nllgrnsfev rvcacpgrdr rteeenlrkk gephhelppg stkralpnnt |
| 181 | ssspqpkkkp ldgeyftlqi rgrerfemfr elnealelkd aqagkepggs rahsshlksk |
| 241 | kgqstsrhkk lmfktegpds d |

Cellular tumor antigen p53, isoform e NP_001119588.1 (SEQ ID NO: 407)

| | |
|---|---|
| 1 | mfcqlaktcp vqlwvdstpp pgtrvramai ykqsqhmtev vrrcphherc sdsdglappq |
| 61 | hlirvegnlr veylddrntf rhsvvvpyep pevgsdctti hynymcnssc mggmnrrpil |
| 121 | tiitledssg nllgrnsfev rvcacpgrdr rteeenlrkk gephhelppg stkralpnnt |
| 181 | ssspqpkkkp ldgeyftlqd qtsfqkenc |

Cellular tumor antigen p53, isoform f NP_001119589.1 (SEQ ID NO: 408)

| | |
|---|---|
| 1 | mfcqlaktcp vqlwvdstpp pgtrvramai ykqsqhmtev vrrcphherc sdsdglappq |
| 61 | hlirvegnlr veylddrntf rhsvvvpyep pevgsdctti hynymcnssc mggmnrrpil |
| 121 | tiitledssg nllgrnsfev rvcacpgrdr rteeenlrkk gephhelppg stkralpnnt |
| 181 | ssspqpkkkp ldgeyftlqm lldlrwcyfl inss |

Cellular tumor antigen p53, isoform g NP_001119590.1, NP_001263689.1,
NP_001263690.1 (SEQ ID NO: 409)

| | |
|---|---|
| 1 | mddlmlspdd ieqwftedpg pdeaprmpea appvapapaa ptpaapapap swplsssvps |
| 61 | qktyqgsygf rlgflhsgta ksvtctyspa lnkmfcqlak tcpvqlwvds tpppgtrvra |
| 121 | maiykqsqhm tevvrrcphh ercsdsdgla ppqhlirveg nlrveylddr ntfrhsvvvp |
| 181 | yeppevgsdc ttihynymcn sscmggmnrr piltiitled ssgnllgrns fevrvcacpg |
| 241 | rdrrteeenl rkkgephhel ppgstkralp nntssspqpk kkpldgeyft lqirgrerfe |
| 301 | mfrelneale lkdaqagkep ggsrahsshl kskkgqstsr hkklmfkteg pdsd |

Cellular tumor antigen p53, isoform h NP_001263624.1 (SEQ ID NO: 410)

| | |
|---|---|
| 1 | mddlmlspdd ieqwftedpg pdeaprmpea appvapapaa ptpaapapap swplsssvps |
| 61 | qktyggsygf rlgflhsgta ksvtctyspa lnkmfcqlak tcpvqlwvds tpppgtrvra |
| 121 | maiykqsqhm tevvrrcphh ercsdsdgla ppqhlirveg nlrveylddr ntfrhsvvvp |
| 181 | yeppevgsdc ttihynymcn sscmggmnrr piltiitled ssgnllgrns fevrvcacpg |
| 241 | rdrrteeenl rkkgephhel ppgstkralp nntssspqpk kkpldgeyft lqmlldlrwc |
| 301 | yflinss |

Cellular tumor antigen p53, isoform i NP_001263625.1 (SEQ ID NO: 411)

| | |
|---|---|
| 1 | mddlmlspdd ieqwftedpg pdeaprmpea appvapapaa ptpaapapap swplsssvps |
| 61 | qktyqgsygf rlgflhsgta ksvtctyspa lnkmfcqlak tcpvqlwvds tpppgtrvra |
| 121 | maiykqsqhm tevvrrcphh ercsdsdgla ppqhlirveg nlrveylddr ntfrhsvvvp |
| 181 | yeppevgsdc ttihynymcn sscmggmnrr piltiitled ssgnllgrns fevrvcacpg |
| 241 | rdrrteeenl rkkgephhel ppgstkralp nntssspqpk kkpldgeyft lqdqtsfqke |
| 301 | nc |

Cellular tumor antigen p53, isoform j NP_001263626.1 (SEQ ID NO: 412)

| | |
|---|---|
| 1 | maiykqsqhm tevvrrcphh ercsdsdgla ppqhlirveg nlrveylddr ntfrhsvvvp |
| 61 | yeppevgsdc ttihynymcn sscmggmnrr piltiitled ssgnllgrns fevrvcacpg |
| 121 | rdrrteeenl rkkgephhel ppgstkralp nntssspqpk kkpldgeyft lqirgrerfe |
| 181 | mfrelneale lkdaqagkep ggsrahsshl kskkgqstsr hkklmfkteg pdsd |

Cellular tumor antigen p53, isoform k NP_001263627.1 (SEQ ID NO: 413)

| | |
|---|---|
| 1 | maiykqsqhm tevvrrcphh ercsdsdgla ppqhlirveg nlrveylddr ntfrhsvvvp |
| 61 | yeppevgsdc ttihynymcn sscmggmnrr piltiitled ssgnllgrns fevrvcacpg |
| 121 | rdrrteeenl rkkgephhel ppgstkralp nntssspqpk kkpldgeyft lqdqtsfqke |
| 181 | nc |

Cellular tumor antigen p53, isoform 1 NP_001263628.1 (SEQ ID NO: 414)

| | |
|---|---|
| 1 | maiykqsqhm tevvrrcphh ercsdsdgla ppqhlirveg nlrveylddr ntfrhsvvvp |
| 61 | yeppevgsdc ttihynymcn sscmggmnrr piltiitled ssgnllgrns fevrvcacpg |
| 121 | rdrrteeenl rkkgephhel ppgstkralp nntssspqpk kkpldgeyft lqmlldlrwc |
| 181 | yflinss |

Dopachrome tautomerase, isoform 1 NP_001913.2 (SEQ ID NO: 415)

| | |
|---|---|
| 1 | msplwwgfll sclgckilpg aqgqfprvcm tvdslvnkec cprlgaesan vcgsqqgrgq |
| 61 | ctevradtrp wsgpyilrnq ddrelwprkf fhrtckctgn fagyncgdck fgwtgpncer |
| 121 | kkppvirqni hslspqereq flgaldlakk rvhpdyvitt qhwlgllgpn gtqpqfancs |
| 181 | vydffvwlhy ysvrdtllgp grpyraidfs hqgpafvtwh ryhllclerd lqrlignesf |
| 241 | alpywnfatg rnecdvctdq lfgaarpddp tlisrnsrfs swetvcdsld dynhlvtlcn |
| 301 | gtyegllrrn qmgrnsmklp tlkdirdcls lqkfdnppff qnstfsfrna legfdkadgt |
| 361 | ldsqvmslhn lvhsflngtn alphsaandp ifvvlhsftd aifdewmkrf nppadawpqe |
| 421 | lapighnrmy nmvpffppvt neelfltsdq lgysyaidlp vsveetpgwp ttllvvmgtl |
| 481 | valvglfvll aflqyrrlrk gytplmethl sskryteea |

Dopachrome tautomerase, isoform 2 NP_001123361.1 (SEQ ID NO: 416)

| | |
|---|---|
| 1 | msplwwgfll sclgckilpg aqgqfprvcm tvdslvnkec cprlgaesan vcgsqqgrgq |
| 61 | ctevradtrp wsgpyilrnq ddrelwprkf fhrtckctgn fagyncgdck fgwtgpncer |
| 121 | kkppvirqni hslspqereq flgaldlakk rvhpdyvitt qhwlgllgpn gtqpqfancs |
| 181 | vydffvwlhy ysvrdtllgp grpyraidfs hqgpafvtwh ryhllclerd lqrlignesf |
| 241 | alpywnfatg rnecdvctdq lfgaarpddp tlisrnsrfs swetvcdsld dynhlvticn |
| 301 | gtyegllrrn qmgrnsmklp tlkdirdcls lqkfdnppff qnstfsfrna legfdkadgt |
| 361 | ldsqvmslhn lvhsflngtn alphsaandp ifvvisnrll ynattnileh vrkekatkel |
| 421 | pslhvlvlhs ftdaifdewm krfnppadaw pqelapighn rmynmvpffp pvtneelflt |
| 481 | sdqlgysyai dlpvsveetp gwpttllvvm gtlvalvglf vllaflqyrr lrkgytplme |
| 541 | thlsskryte ea |

Dopachrome tautomerase, isoform 3 NP_001309lll.1, NP_001309112.1,
NP_001309113.1, NP_ 001309114.1 (SEQ ID NO: 417)

| | |
|---|---|
| 1 | mgrnsmklpt lkdirdclsl qkfdnppffq nstfsfrnal egfdkadgtl dsqvmslhnl |
| 61 | vhsflngtna lphsaandpi fvvlhsftda ifdewmkrfn ppadawpqel apighnrmyn |
| 121 | mvpffppvtn eelfltsdql gysyaidlpv sveetpgwpt tllvvmgtlv alvglfvlla |
| 181 | flqyrrlrkg ytplmethls skryteea |

Dopachrome tautomerase, isoform 4, NP_001309115.1 (SEQ ID NO: 418)

| | |
|---|---|
| 1 | mllgiqrqmk crlrsdvtkr leedehvnth spmrrgnfag yncgdckfgw tgpncerkkp |
| 61 | pvirqnihsl spqereqflg aldlakkrvh pdyvittqhw lgllgpngtq pqfancsvyd |
| 121 | ffvwlhyysv rdtllgpgrp yraidfshqg pafvtwhryh llclerdlqr lignesfalp |
| 181 | ywnfatgrne cdvctdqlfg aarpddptli srnsrfsswe tvcdslddyn hlvtlcngty |
| 241 | egllrrnqmg rnsmklptlk dirdclslqk fdnppffqns tfsfrnaleg fdkadgtlds |
| 301 | qvmslhnlvh sflngtnalp hsaandpifv vlhsftdaif dewmkrfnpp adawpqelap |
| 361 | ighnrmynmv pffppvtnee lfltsdqlgy syaidlpvsv eetpgwpttl lvvmgtlval |
| 421 | vglfvllafl qyrrlrkgyt plmethlssk ryteea |

Transformation/transcription domain associated protein, isoform 1
NP_001231509.1 (SEQ ID NO: 419)

| | |
|---|---|
| 1 | mafvatqgat vvdqttlmkk ylqfvaaltd vntpdetklk mmqevsenfe nvtsspqyst |
| 61 | flehiiprfl tflqdgevqf lqekpaqqlr klvleiihri ptnehlrpht knvlsvmfrf |
| 121 | leteneenvl iclriiielh kqfrppitqe ihhfldfvkq iykelpkvvn ryfenpqvip |
| 181 | entvpppemv gmittiavkv nperedsetr thsiiprgsl slkvlaelpi ivvlmyqlyk |
| 241 | lnihnvvaef vplimntiai qvsaqarqhk lynkelyadf iaaqiktlsf layiiriyqe |
| 301 | lvtkysqqmv kgmlqllsnc paetahlrke lliaakhilt telrnqfipc mdklfdesil |
| 361 | igsgytaret lrplaystla dlvhhvrqhl plsdlslavq lfakniddes lpssiqtmsc |
| 421 | klllnlvdci rskseqesgn grdvlmrmle vfvlkfhtia ryqlsaifkk ckpqselgav |
| 481 | eaalpgvpta paapgpapsp apvpappppp pppppatpvt papvppfekq gekdkedkqt |
| 541 | fqvtdcrslv ktlvcgvkti twgitsckap geaqfipnkq lqpketqiyi klvkyamqal |
| 601 | diyqvqiagn gqtyirvanc qtvrmkeeke vlehfagvft mmnpltfkei fqttvpymve |
| 661 | risknyalqi vansflanpt tsalfatilv eylldrlpem gsnvelsnly lklfklvfgs |
| 721 | vslfaaeneq mlkphlhkiv nssmelaqta kepynyflll ralfrsiggg shdllyqefl |
| 781 | pllpnllqgl nmlqsglhkq hmkdlfvelc ltvpvrlssl lpylpmlmdp lvsalngsqt |
| 841 | lvsqglrtle lcvdnlqpdf lydhiqpvra elmqalwrtl rnpadsishv ayrvlgkfgg |
| 901 | snrkmlkesq klhyvvtevq gpsitvefsd ckaslqlpme kaietaldcl ksantepyyr |
| 961 | rqawevikof lvammsledn khalyqllah pnftektipn viishrykaq dtparktfeq |
| 1021 | altgafmsav ikdlrpsalp fvaslirhyt mvavaqqcgp fllpcyqvgs qpstamfhse |
| 1081 | engskgmdpl vlidaiaicm ayeekelcki gevalavifd vasiilgske racqlplfsy |
| 1141 | iverlcaccy eqawyaklgg vvsikflmer lpltwvlqnq qtflkallfv mmdltgevsn |
| 1201 | gavamakttl eqllmrcatp lkdeeraeei vaaqeksfhh vthdlvrevt spnstvrkqa |
| 1261 | mhslqvlaqv tgksvtvime phkevlqdmv ppkkhllrhq panaqiglme gntfcttlqp |
| 1321 | rlftmdlnvv ehkvfytell nlceaedsal tklpcykslp slvplriaal nalaacnylp |
| 1381 | qsrekiiaal fkalnstnse lqeageacmr kflegatiev dqihthmrpl lmmlgdyrsl |
| 1441 | tlnvvnrlts vtrlfpnsfn dkfcdqmmqh lrkwmevvvi thkggqrsdg nesisecgrc |
| 1501 | plspfcqfee mkicsaiinl fhlipaapqt lvkpllevvm kteramliea gspfreplik |
| 1561 | fltrhpsqtv elfmmeatln dpqwsrmfms flkhkdarpl rdvlaanpnr fitlllpgga |
| 1621 | qtavrpgsps tstmrldlqf qaikiisiiv knddswlasq hslvsqlrry wvsenfqerh |
| 1681 | rkenmaatnw kepkllaycl lnyckrnygd iellfqllra ftgrflcnmt flkeymeeei |
| 1741 | pknysiaqkr alffrfvdfn dpnfgdelka kvlqhilnpa flysfekgeg eqllgppnpe |
| 1801 | gdnpesitsv fitkvldpek qadmldslri yllqyatllv ehaphhihdn nknrnsklrr |
| 1861 | lmtfawpc11 skacvdpack ysghlllahi iakfaihkki vlqvfhsllk ahamearaiv |
| 1921 | rqamailtpa vparmedghq mlthwtrkii veeghtvpql vhilhlivqh fkvyypvrhh |
| 1981 | lvqhmvsamq rlgftpsvti eqrrlavdls evvikwelqr ikdqqpdsdm dpnssgegvn |
| 2041 | sysssikrgl svdsaqevkr frtatgaisa vfgrsqslpg adsllakpid kqhtdtvvnf |
| 2101 | lirvacqvnd ntntagspge vlsrrcvnll ktalrpdmwp kselklqwfd kllmtveqpn |
| 2161 | qvnygnictg levlsflltv lqspailssf kplqrgiaac mtcgntkvlr avhsllsrlm |
| 2221 | sifptepsts svaskyeele clyaavgkvi yegltnyeka tnanpsqlfg tlmilksacs |
| 2281 | nnpsyidrli svfmrslqkm vrehlnpqaa sgsteatsgt selvmlslel vktrlavmsm |
| 2341 | emrknfiqai ltsliekspd akilravvki veewvknnsp maanqtptlr eksillvkmm |
| 2401 | tyiekrfped lelnaqfldl vnyvyrdetl sgseltakle paflsglrca qplirakffe |
| 2461 | vfdnsmkrrv yerllyvtcs qnweamgnhf wikqcielll avcekstpig tscqgamlps |
| 2521 | itnvinlads hdraafamvt hvkqeprere nseskeedve idielapgdq tstpktkels |
| 2581 | ekdignqlhm ltnrhdkfld tlrevktgal lsafvqlchi sttlaektwv qlfprlwkil |
| 2641 | sdrqqhalag eispflcsgs hqvqrdcqps alncfveams qcvppipirp cvlkylgkth |
| 2701 | nlwfrstlml ehqafekgls lqikpkqtte fyeqesitpp qqeildslae lysllqeedm |
| 2761 | waglwqkrck ysetataiay eqhgffeqaq esyekamdka kkehersnas paifpeyqlw |
| 2821 | edhwircske lnqwealtey gqskghinpy lvlecawrvs nwtamkealv qvevscpkem |
| 2881 | awkvnmyrgy laichpeeqq lsfierlvem asslairewr rlphvvshvh tpllqaaqqi |
| 2941 | ielqeaaqin aglqptnlgr nnslhdmktv vktwrnrlpi vsddlshwss ifmwrqhhyq |
| 3001 | gkptwsgmhs ssivtayens sqhdpssnna mlgvhasasa iiqygkiark qglvnvaldi |
| 3061 | lsrihtiptv pivdcfqkir qqvkcylqla gvmgknecmq gleviestnl kyftkemtae |
| 3121 | fyalkgmfla qinkseeank afsaavqmhd vlvkawamwg dylenifvke rqlhlgvsai |
| 3181 | tcylhacrhq nesksrkyla kvlwllsfdd dkntladavd kycigvppiq wlawipqllt |
| 3241 | clvgsegkll lnlisqvgrv ypqavyfpir tlyltlkieq reryksdpgp iratapmwrc |
| 3301 | srimhmqrel hptllssleg ivdqmvwfre nwheevlrql qqglakcysv afeksgaysd |
| 3361 | akitphtlnf vkklvstfgv glenvsnvst mfssaasesl arraqataqd pvfqklkgqf |
| 3421 | ttdfdfsvpg smklhnlisk lkkwikilea ktkqlpkffl ieekcrflsn fsaqtaevei |
| 3481 | pgeflmpkpt hyyikiarfm prveivqkhn taarrlyirg hngkiypylv mndacltesr |
| 3541 | reervlqllr llnpclekrk ettkrhlfft vprvvayspq mrlvednpss lslveiykqr |
| 3601 | cakkgiehdn pisryydrla tvqargtqas hqvlrdilke vqsnmvprsm lkewalhtfp |
| 3661 | natdywtfrk mftiqlalig faefv1h1nr lnpemlqiaq dtgklnvayf rfdindatgd |
| 3721 | ldanrpvpfr ltpniseflt tigvsgplta smiavarcfa qpnfkvdgil ktvlrdeiia |
| 3781 | whkktqedts splsaagqpe nmdsqqlvsl vqkavtaimt rlhnlaqfeg geskvntiva |
| 3841 | aansldnlcr mdpawhpwl |

Transformation/transcription domain associated protein, isoform 2 NP_003487.1
(SEQ ID NO: 420)

| | |
|---|---|
| 1 | mafvatqgat vvdqttlmkk ylqfvaaltd vntpdetklk mmqevsenfe nvtsspqyst |
| 61 | flehiiprfl tflqdgevqf lqekpaqqlr klvleiihri ptnehlrpht knvlsvmfrf |
| 121 | leteneenvl iclriiielh kqfrppitqe ihhfldfvkq iykelpkvvn ryfenpqvip |
| 181 | entvpppemv gmittiavkv nperedsetr thsiiprgsl slkvlaelpi ivvlmyqlyk |
| 241 | lnihnvvaef vplimntiai qvsaqarqhk lynkelyadf iaaqiktlsf layiiriyqe |
| 301 | lvtkysqqmv kgmlqllsnc paetahlrke lliaakhilt telrnqfipc mdklfdesil |
| 361 | igsgytaret lrplaystla dlvhhvrqhl plsdlslavq lfakniddes lpssiqtmsc |
| 421 | klllnlvdci rskseqesgn grdvlmrmle vfvlkfhtia ryqlsaifkk ckpqselgav |
| 481 | eaalpgvpta paapgpapsp apvpappppp pppppatpvt papvppfekq gekdkedkqt |
| 541 | fqvtdcrslv ktlvcgvkti twgitsckap geaqfipnkq lqpketqiyi klvkyamqal |
| 601 | diyqvqiagn gqtyirvanc qtvrmkeeke vlehfagvft mmnpltfkei fqttvpymve |
| 661 | risknyalqi vansflanpt tsalfatilv eylldrlpem gsnvelsnly lklfklvfgs |
| 721 | vslfaaeneq mlkphlhkiv nssmelaqta kepynyflll ralfrsiggg shdllyqefl |
| 781 | pllpnllqgl nmlqsglhkq hmkdlfvelc ltvpvrlssl lpylpmlmdp lvsalngsqt |
| 841 | lvsqglrtle lcvdnlqpdf lydhiqpvra elmqalwrtl rnpadsishv ayrvlgkfgg |
| 901 | snrkmlkesq klhyvvtevq gpsitvefsd ckaslqlpme kaietaldcl ksantepyyr |
| 961 | rqawevikcf lvammsledn khalyqllah pnftektipn viishrykaq dtparktfeq |
| 1021 | altgafmsav ikdlrpsalp fvaslirhyt mvavaqqcgp fllpcyqvgs qpstamfhse |
| 1081 | engskgmdpl vlidaiaicm ayeekelcki gevalavifd vasiilgske racqlplfsy |
| 1141 | iverlcaccy eqawyaklgg vvsikflmer lpltwvlqnq qtflkallfv mmdltgevsn |
| 1201 | gavamakttl eqllmrcatp lkdeeraeei vaaqeksfhh vthdlvrevt spnstvrkqa |
| 1261 | mhslqvlaqv tgksvtvime phkevlqdmv ppkkhllrhq panaqiglme gntfcttlqp |
| 1321 | rlftmdlnvv ehkvfytell nlceaedsal tklpcykslp slvplriaal nalaacnylp |
| 1381 | qsrekiiaal fkalnstnse lqeageacmr kflegatiev dqihthmrpl lmmlgdyrsl |
| 1441 | tlnvvnrlts vtrlfpnsfn dkfcqgmmqh lrkwmevvvi thkggqrsdg nemkicsaii |
| 1501 | nlfhlipaap qtlvkpllev vmkteramli eagspfrepl ikfltrhpsq tvelfmmeat |
| 1561 | lndpqwsrmf msflkhkdar plrdvlaanp nrfitlllpg gaqtavrpgs pststmrldl |
| 1621 | qfqaikiisi ivknddswla sqhslvsqlr rvwvsenfqe rhrkenmaat nwkepkllay |
| 1681 | cllnyckrny gdiellfqll raftgrflcn mtflkeymee eipknysiaq kralffrfvd |
| 1741 | fndpnfgdel kakvlqhiln paflysfekg egegllgppn pegdnpesit svfitkvldp |
| 1801 | ekqadmldsl riyllqyatl lvehaphhih dnnknrnskl rrlmtfawpc llskacvdpa |
| 1861 | ckysghllla hiiakfaihk kivlqvfhsl lkahameara ivrqamailt pavparmedg |
| 1921 | hqmlthwtrk iiveeghtvp qlvhilhliv qhfkvyypvr hhlvqhmvsa mqrlgftpsv |
| 1981 | tieqrrlavd lsevvikwel grikdqqpds dmdpnssgeg vnsysssikr glsvdsaqev |
| 2041 | krfrtatgai savfgrsqsl pgadsllakp idkqhtdtvv nflirvacqv ndntntagsp |
| 2101 | gevlsrrcvn llktalrpdm wpkselklqw fdkllmtveq pnqvnygnic tglevlsfll |
| 2161 | tvlqspails sfkplqrgia acmtcgntkv lravhsllsr lmsifpteps tssvaskyee |
| 2221 | leclyaavgk viyegltnye katnanpsql fgtlmilksa csnnpsyidr lisvfmrslq |
| 2281 | kmvrehlnpq aasgsteats gtselvmlsl elvktrlavm smemrknfiq ailtslieks |
| 2341 | pdakilravv kiveewvknn spmaanqtpt lreksillvk mmtyiekrfp edlelnaqfl |
| 2401 | dlvnyvyrde tlsgseltak lepaflsglr caqplirakf fevfdnsmkr rvyerllyvt |
| 2461 | csqnweamgn hfwikqciel llavcekstp igtscqgaml psitnvinla dshdraafam |
| 2521 | vthvkqepre renseskeed veidielapg dqtstpktke lsekdignql hmltnrhdkf |
| 2581 | ldtlrevktg allsafvqlc histtlaekt wvqlfprlwk ilsdrqqhal ageispflcs |
| 2641 | gshqvqrdcq psalncfvea msqcvppipi rpcvlkylgk thnlwfrstl mlehqafekg |
| 2701 | lslqikpkqt tefyeqesit ppqqeildsl aelysllqee dmwaglwqkr ckysetatai |
| 2761 | ayeqhgffeq aqesyekamd kakkehersn aspaifpeyq lwedhwircs kelnqwealt |
| 2821 | eygqskghin pylvlecawr vsnwtamkea lvqvevscpk emawkvnmyr gylaichpee |
| 2881 | qqlsfierlv emasslaire wrrlphvvsh vhtpllqaaq qiielqeaaq inaglqptnl |
| 2941 | grnnslhdmk tvvktwrnrl pivsddlshw ssifmwrqhh yqaivtayen ssqhdpssnn |
| 3001 | amlgvhasas aiiqygkiar kqglvnvald ilsrihtipt vpivdcfqki rqqvkcylql |
| 3061 | agvmgknecm qgleviestn lkyftkemta efyalkgmfl aqinkseean kafsaavqmh |
| 3121 | dvlvkawamw gdylenifvk erqlhlgvsa itcylhacrh qnesksrkyl akvlwllsfd |
| 3181 | ddkntladav dkyvigvppi qwlawipqll tclvgsegkl llnlisqvgr vypqavyfpi |
| 3241 | rtlyltlkie qreryksdpg piratapmwr csrimhmqre lhptllssle givdqmvwfr |
| 3301 | enwheevlrq lqqglakcys vafeksgavs dakitphtln fvkklvstfg vglenvsnvs |
| 3361 | tmfssaases larraqataq dpvfqklkgq fttdfdfsvp gsmklhnlis klkkwikile |
| 3421 | aktkqlpkff lieekcrfls nfsaqtaeve ipgeflmpkp thyyikiarf mprveivqkh |
| 3481 | ntaarrlyir ghngkiypyl vmndacltes rreervlqll rllnpclekr kettkrhlff |
| 3541 | tvprvvaysp qmrlvednps slslveiykg rcakkgiehd npisryydrl atvqargtqa |
| 3601 | shqvlrdilk evqsnmvprs mlkewalhtf pnatdywtfr kmftiqlali gfaefvlhln |
| 3661 | rlnpemlqia qdtgklnvay frfdindatg dldanrpvpf rltpnisefl ttigvsgplt |
| 3721 | asmiavarcf aqpnfkvdgi lktvlrdeii awhkktqedt ssplsaagqp enmdsqqlvs |
| 3781 | lvqkavtaim trlhnlaqfe ggeskvntlv aaansldnlc rmdpawhpwl |

Tyrosinase precursor NP_000363.1 (SEQ ID NO: 421)

| | |
|---|---|
| 1 | mllavlycll wsfqtsaghf pracvssknl mekeccppws gdrspcgqls grgscqnill |
| 61 | snaplgpqfp ftgvddresw psvfynrtcq csgnfmgfnc gnckfgfwgp ncterrllvr |
| 121 | rnifdlsape kdkffayltl akhtissdyv ipigtygqmk ngstpmfndi niydlfvwmh |
| 181 | yyvsmdallg gseiwrdidf aheapaflpw hrlfllrweq eiqkltgden ftipywdwrd |
| 241 | aekcdictde ymggqhptnp nllspasffs swqivcsrle eynshqslcn gtpegplrrn |
| 301 | pgnhdksrtp rlpssadvef clsltqyesg smdkaanfsf rntlegfasp ltgiadasqs |
| 361 | smhnalhiym ngtmsqvqgs andpifllhh afvdsifeqw lrrhrplqev ypeanapigh |
| 421 | nresymvpfi plyrngdffi sskdlgydys ylqdsdpdsf qdyiksyleq asriwswllg |
| 481 | aamvgavlta llaglvsllc rhkrkqlpee kqpllmeked yhslyqshl |

Vascular endothelial growth factor A, isoform a NP_001020537.2 (SEQ ID NO:
422)

| | |
|---|---|
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kksvrgkgkg qkrkrkksry kswsvyvgar cclmpwslpg |
| 361 | phpcgpcser rkhlfvqdpq tckcsckntd srckarqlel nertcrcdkp rr |

Vascular endothelial growth factor A, isoform b NP_003367.4 (SEQ ID NO: 423)

| | |
|---|---|
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kksvrgkgkg qkrkrkksry kswsvpcgpc serrkhlfvq |
| 361 | dpqtckcsck ntdsrckarq lelnertcrc dkprr |

Vascular endothelial growth factor A, isoform c NP_001020538.2 (SEQ ID NO:
424)

| | |
|---|---|
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kksvrgkgkg qkrkrkksrp cgpcserrkh lfvqdpqtck |
| 361 | csckntdsrc karqlelner tcrcdkprr |

Vascular endothelial growth factor A, isoform d NP_001020539.2 (SEQ ID NO:
425)

| | |
|---|---|
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe npcgpcserr khlfvqdpqt ckcsckntds rckarqleln |
| 361 | ertcrcdkpr r |

Vascular endothelial growth factor A, isoform e NP_001020540.2 (SEQ ID NO:
426)

| | |
|---|---|
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe npcgpcserr khlfvgdpqt ckcsckntds rckm |

Vascular endothelial growth factor A, isoform f NP_001020541.2 (SEQ ID NO:
427)

| | |
|---|---|
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kcdkprr |

Vascular endothelial growth factor A, isoform g NP_001028928.1 (SEQ ID NO:
428)

| | |
|---|---|
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe npcgpcserr khlfvgdpqt ckcsckntds rckarqleln |
| 361 | ertcrsltrk d |

Vascular endothelial growth factor A, isoform h NP_001165093.1 (SEQ ID NO:
429)

| | |
|---|---|
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rcdkprr |

Vascular endothelial growth factor A, isoform i NP_001165094.1 (SEQ ID NO:
430)

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kksvrgkgkg qkrkrkksry kswsvyvgar cclmpwslpg |
| 181 | phpcgpcser rkhlfvqdpq tckcsckntd srckarqlel nertcrcdkp rr |

Vascular endothelial growth factor A, isoform j NP_001165095.1 (SEQ ID NO:
431)

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kksvrgkgkg qkrkrkksry kswsvpcgpc serrkhlfvq |
| 181 | dpqtckcsck ntdsrckarq lelnertcrc dkprr |

Vascular endothelial growth factor A, isoform k NP_001165096.1 (SEQ ID NO:
432)

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kksvrgkgkg qkrkrkksrp cgpcserrkh lfvqdpqtck |
| 181 | csckntdsrc karqlelner tcrcdkprr |

Vascular endothelial growth factor A, isoform 1 NP_001165097.1 (SEQ ID NO:
433)

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe npcgpcserr khlfvqdpqt ckcsckntds rckarqleln |
| 181 | ertcrcdkpr r |

Vascular endothelial growth factor A, isoform m NP_001165098.1 (SEQ ID NO:
434)

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe npcgpcserr khlfvqdpqt ckcsckntds rckm |

Vascular endothelial growth factor A, isoform n NP_001165099.1 (SEQ ID NO:
435)

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kcdkprr |

Vascular endothelial growth factor A, isoform o NP_001165100.1 (SEQ ID NO:
436)

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe npcgpcserr khlfvqdpqt ckcsckntds rckarqleln |
| 181 | ertcrsltrk d |

Vascular endothelial growth factor A, isoform p NP_001165101.1 (SEQ ID NO:
437)

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rcdkprr |

Vascular endothelial growth factor A, isoform q NP_001191313.1 (SEQ ID NO:
438)

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kksvrgkgkg qkrkrkksry kswsvcdkpr r |

Vascular endothelial growth factor A, isoform r NP_001191314.1 (SEQ ID NO:
439)

| | |
|---|---|
| 1 | mtdrqtdtap spsyhllpgr rrtvdaaasr gqgpepapgg gvegvgargv alklfvqllg |
| 61 | csrfggavvr ageaepsgaa rsassgreep qpeegeeeee keeergpqwr lgarkpgswt |
| 121 | geaavcadsa paarapqala rasgrggrva rrgaeesgpp hspsrrgsas ragpgraset |
| 181 | mnfllswvhw slalllylhh akwsqaapma egggqnhhev vkfmdvyqrs ychpietlvd |
| 241 | ifqeypdeie yifkpscvpl mrcggccnde glecvptees nitmqimrik phqgqhigem |
| 301 | sflqhnkcec rpkkdrarqe kksvrgkgkg qkrkrkksry kswsvcdkpr r |

Vascular endothelial growth factor A, isoform s NP_001273973.1 (SEQ ID NO:
440)

| | |
|---|---|
| 1 | maegggqnhh evvkfmdvyq rsychpietl vdifqeypde ieyifkpscv plmrcggccn |
| 61 | deglecvpte esnitmqimr ikphqgqhig emsflqhnkc ecrpkkdrar qenpcgpcse |
| 121 | rrkhlfvqdp qtckcscknt dsrckarqle lnertcrcdk prr |

Vascular endothelial growth factor A, isoform VEGF-Ax precursor
NP_001303939.1 (SEQ ID NO: 441)

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe npcgpcserr khlfvqdpqt ckcsckntds rckarqleln |
| 181 | ertcrcdkpr rsagqeegas lrvsgtrslt rkd |

WD repeat-containing protein 46, isoform 1 NP_005443.3 (SEQ ID NO: 442)

| | |
|---|---|
| 1 | metapkpgkd vppkkdklqt krkkprrywe eetvpttaga spgpprnkkn relrpqrpkn |
| 61 | ayilkksris kkpqvpkkpr ewknpesqrg lsgtqdpfpg papvpvevvq kfcridksrk |
| 121 | lphskaktrs rlevaeaeee etsikaarse lllaeepgfl egedgedtak icqadiveav |
| 181 | diasaakhfd lnlrqfgpyr lnysrtgrhl afggrrghva aldwvtkklm ceinvmeavr |
| 241 | dirflhseal lavaqnrwlh iydnqgielh cirrcdrvtr leflpfhfll atasetgflt |
| 301 | yldvsvgkiv aalnaragrl dvmsqnpyna vihlghsngt vslwspamke plakilchrg |
| 361 | gvravavdst gtymatsgld hqlkifdlrg tyqplstrtl phgaghlafs qrgllvagmg |
| 421 | dvvniwagqg kasppsleqp ylthrlsgpv hglqfcpfed vlgvghtggi tsmlvpgage |
| 481 | pnfdglesnp yrsrkqrqew evkallekvp aelicldpra laevdvisle qgkkeqierl |
| 541 | gydpqakapf qpkpkqkgrs staslvkrkr kvmdeehrdk vrqslqqqhh keakakptga |
| 601 | rpsaldrfvr |

WD repeat-containing protein 46, isoform 2 NP_001157739.1 (SEQ ID NO: 443)

| | |
|---|---|
| 1 | metapkpgkd vppkkdklqt krkkprewkn pesqrglsgt qdpfpgpapv pvevvqkfcr |
| 61 | idksrklphs kaktrsrlev aeaeeeetsi kaarsellla eepgfleged gedtakicqa |
| 121 | diveavdias aakhfdlnlr qfgpyrlnys rtgrhlafgg rrghvaaldw vtkklmcein |
| 181 | vmeavrdirf lhseallava qnrwlhiydn qgielhcirr cdrvtrlefl pfhfllatas |
| 241 | etgfltyldv svgkivaaln aragrldvms qnpynavihl ghsngtvslw spamkeplak |
| 301 | ilchrggvra vavdstgtym atsgldhqlk ifdlrgtyqp lstrtlphga ghlafsqrgl |
| 361 | lvagmgdvvn iwagqgkasp psleqpylth rlsgpvhglq fcpfedvlgv ghtggitsml |
| 421 | vpgagepnfd glesnpyrsr kqrqewevka llekvpaeli cldpralaev dvisleqgkk |
| 481 | eqierlgydp qakapfqpkp kqkgrsstas lvkrkrkvmd eehrdkvrqs lqqqhhkeak |
| 541 | akptgarpsa ldrfvr |

Wilms tumor protein, isoform A NP_000369.4 (SEQ ID NO: 444)

| | |
|---|---|
| 1 | mdflllqdpa stcvpepasq htlrsgpgcl qqpeqqgvrd pggiwaklga aeasaerlqg |
| 61 | rrsrgasgse pqqmgsdvrd lnallpavps lgggggcalp vsgaaqwapv ldfappgasa |
| 121 | ygslggpapp papppppppp phsfikqeps wggaepheeq clsaftvhfs gqftgtagac |
| 181 | rygpfgpppp sqassgqarm fpnapylpsc lesqpairnq gystvtfdgt psyghtpshh |
| 241 | aaqfpnhsfk hedpmgqqgs lgeqqysvpp pvygchtptd sctgsqalll rtpyssdnly |
| 301 | qmtsqlecmt wnqmnlgatl kghstgyesd nhttpilcga qyrihthgvf rgiqdvrrvp |
| 361 | gvaptlvrsa setsekrpfm caypgcnkry fklshlqmhs rkhtgekpyq cdfkdcerrf |
| 421 | srsdqlkrhq rrhtgvkpfq cktcqrkfsr sdhlkthtrt htgekpfscr wpscqkkfar |
| 481 | sdelvrhhnm hqrnmtklql al |

Wilms tumor protein, isoform B NP_077742.3 (SEQ ID NO: 445)

| | |
|---|---|
| 1 | mdflllqdpa stcvpepasq htlrsgpgcl qqpeqqgvrd pggiwaklga aeasaerlqg |
| 61 | rrsrgasgse pqqmgsdvrd lnallpavps lgggggcalp vsgaaqwapv ldfappgasa |
| 121 | ygslggpapp papppppppp phsfikqeps wggaepheeq clsaftvhfs gqftgtagac |
| 181 | rygpfgpppp sqassgqarm fpnapylpsc lesqpairnq gystvtfdgt psyghtpshh |
| 241 | aaqfpnhsfk hedpmgqqgs lgeqqysvpp pvygchtptd sctgsqalll rtpyssdnly |
| 301 | qmtsqlecmt wnqmnlgatl kgvaagssss vkwtegqsnh stgyesdnht tpilcgaqyr |
| 361 | ihthgvfrgi qdvrrvpgva ptlvrsaset sekrpfmcay pgcnkryfkl shlqmhsrkh |
| 421 | tgekpyqcdf kdcerrfsrs dqlkrhqrrh tgvkpfqckt cqrkfsrsdh lkthtrthtg |
| 481 | ekpfscrwps cqkkfarsde lvrhhnmhqr nmtklqlal |

Wilms tumor protein, isoform D NP_077744.4 (SEQ ID NO: 446)

| | |
|---|---|
| 1 | mdflllqdpa stcvpepasq htlrsgpgcl qqpeqqgvrd pggiwaklga aeasaerlqg |
| 61 | rrsrgasgse pqqmgsdvrd lnallpavps lgggggcalp vsgaaqwapv ldfappgasa |
| 121 | ygslggpapp papppppppp phsfikqeps wggaepheeq clsaftvhfs gqftgtagac |
| 181 | rygpfgpppp sgassgqarm fpnapylpsc lesqpairnq gystvtfdgt psyghtpshh |
| 241 | aaqfpnhsfk hedpmgqqgs lgeqqysvpp pvygchtptd sctgsqalll rtpyssdnly |
| 301 | qmtsqlecmt wnqmnlgatl kgvaagssss vkwtegqsnh stgyesdnht tpilcgaqyr |
| 361 | ihthgvfrgi qdvrrvpgva ptlvrsaset sekrpfmcay pgcnkryfkl shlqmhsrkh |
| 421 | tgekpyqcdf kdcerrfsrs dqlkrhqrrh tgvkpfqckt cqrkfsrsdh lkthtrthtg |
| 481 | ktsekpfscr wpscqkkfar sdelvrhhnm hqrnmtklql al |

Wilms tumor protein, isoform E NP_001185480.1 (SEQ ID NO: 447)

| | |
|---|---|
| 1 | mekgystvtf dgtpsyghtp shhaaqfpnh sfkhedpmgq qgslgeqqys vpppvygcht |
| 61 | ptdsctgsqa lllrtpyssd nlyqmtsqle cmtwnqmnlg atlkgvaags sssvkwtegq |
| 121 | snhstgyesd nhttpilcga qyrihthgvf rgiqdvrrvp gvaptlvrsa setsekrpfm |
| 181 | caypgcnkry fklshlqmhs rkhtgekpyq cdfkdcerrf srsdqlkrhq rrhtgvkpfq |
| 241 | cktcqrkfsr sdhlkthtrt htgekpfscr wpscqkkfar sdelvrhhnm hqrnmtklql |
| 301 | al |

Wilms tumor protein, isoform F NP_001185481.1 (SEQ ID NO: 448)

| | |
|---|---|
| 1 | mekgystvtf dgtpsyghtp shhaaqfpnh sfkhedpmgq qqslgeqqys vpppvygcht |
| 61 | ptdsctgsqa lllrtpyssd nlyqmtsqle cmtwnqmnlg atlkghstgy esdnhttpil |
| 121 | cgaqyrihth gvfrgiqdvr rvpgvaptlv rsasetsekr pfmcaypgcn kryfklshlq |
| 181 | mhsrkhtgek pyqcdfkdce rrfsrsdqlk rhqrrhtgvk pfqcktcqrk fsrsdhlkth |
| 241 | trthtgktse kpfscrwpsc qkkfarsdel vrhhnmhqrn mtklqlal |

X antigen family member 1, isoform a NP_001091063.2 (SEQ ID NO: 449)

| | |
|---|---|
| 1 | mespkkknqq lkvgilhlgs rqkkiriqlr sqcatwkvic kscisqtpgi nldlgsgvkv |
| 61 | kiipkeehck mpeageeqpq v |

X antigen family member 1, isoform d NP_001091065.1 (SEQ ID NO: 450)

| | |
|---|---|
| 1 | mespkkknqq lkvgilhlgs rqkkiriqlr sqvlgremrd megdlqelhq sntgdksgfg |
| 61 | frrqgednt |

X-linked inhibitor of apoptosis NP_001158.2, NP_001191330.1 (SEQ ID NO: 451)

| | |
|---|---|
| 1 | mtfnsfegsk tcvpadinke eefveefnrl ktfanfpsgs pvsastlara gflytgegdt |
| 61 | vrcfschaav drwqygdsav grhrkvspnc rfingfylen satqstnsgi qngqykveny |
| 121 | lgsrdhfald rpsethadyl lrtgqvvdis dtiyprnpam yseearlksf qnwpdyahlt |
| 181 | prelasagly ytgigdqvqc fccggklknw epcdrawseh rrhfpncffv lgrnlnirse |
| 241 | sdayssdrnf pnstnlprnp smadyearif tfgtwiysvn keqlaragfy algegdkvkc |
| 301 | fhcgggltdw kpsedpweqh akwypgckyl leqkgqeyin nihlthslee clvrttektp |
| 361 | sltrriddti fqnpmvqeai rmgfsfkdik kimeekiqis gsnykslevl vadlvnaqkd |
| 421 | smgdessqts lqkeisteeq lrrlqeeklc kicmdrniai vfvpcghlvt ckqcaeavdk |
| 481 | cpmcytvitf kqkifms |

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims:

We claim:

1. A method of obtaining a plurality of lymphocytes selectively stimulated by one or more stimulatory antigens, the method comprising:
obtaining a sample of PBMCs from a subject having a tumor or a cancer;
isolating from the sample of PBMCs a population of lymphocytes (e.g., T cells) and a population of monocytes;
differentiating the monocytes into dendritic cells;
selecting one or more stimulatory antigens by:
a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject;
b) contacting the bacterial cells or beads with antigen presenting cells (APCs) (e.g., a first batch of the dendritic cells) from the subject, wherein the APCs internalize the bacterial cells or beads;
c) contacting the APCs with a first batch of the population of lymphocytes from the subject, under conditions suitable for activation of lymphocytes by a polypeptide presented by one or more APCs;
d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer;
co-culturing a second batch of the dendritic cells with (i) a second batch of the population of lymphocytes (e.g., T cells), and (ii) a plurality of overlapping peptides, wherein the overlapping peptides comprise all or part of the amino acid sequence of the one or more stimulatory antigens; and
selecting or enriching from the culture a plurality of lymphocytes (e.g., T cells) selectively stimulated by the one or more stimulatory antigens.

2. The method of claim 1, wherein the population of monocytes comprises CD14⁺ monocytes.

3. The method of claim 1 or 2, wherein the population of lymphocytes (e.g., T cells) comprises CD4⁺ and/or CD8⁺ T cells.

4. The method of any one of claims 1-3, further comprising expanding and/or restimulating the plurality of lymphocytes (e.g., T cells).

5. The method of claim 4, wherein expanding and/or restimulating comprises contacting the plurality of lymphocytes (e.g., T cells) with the plurality of overlapping peptides.

6. The method of claim 4 or 5, further comprising selecting or enriching, from the plurality of lymphocytes (e.g., T cells), lymphocytes (e.g., T cells) selectively expanded and.or stimulated by the one or more stimulatory antigens.

7. The method of any one of claims 4-6, further comprising non-selectively expanding and/or stimulating the selected or enriched lymphocytes (e.g., T cells).

8. The method of claim 7, wherein non-selectively expanding and/or stimulating comprises contacting the plurality of lymphocytes (e.g., T cells) with an anti-CD3 antibody, an anti-CD28 antibody, and/or an anti-CD2 antibody.

9. The method of claim 8, further comprising formulating the plurality of lymphocytes (e.g., T cells) as a composition.

10. The method of claim 9, wherein the composition comprises a diluent and human serum albumin.

11. The method of claim 10, wherein the composition is cryo-preserved.

12. A method of manufacturing a pharmaceutical composition, the method comprising:
obtaining a sample of PBMCs from a subject having a tumor or a cancer;
isolating from the sample of PBMCs a population of lymphocytes (e.g., T cells) and a population of monocytes;
differentiating the monocytes into dendritic cells;
selecting one or more stimulatory antigens by:
a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject;
b) contacting the bacterial cells or beads with antigen presenting cells (APCs) (e.g., a first batch of the dendritic cells), wherein the APCs internalize the bacterial cells or beads;
c) contacting the APCs with a first batch of the population of lymphocytes, under conditions suitable for activation of lymphocytes by a polypeptide presented by one or more APCs;
d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer;
co-culturing a second batch of the dendritic cells with (i) a second batch of the population of lymphocytes (e.g., T cells), and (ii) a plurality of overlapping peptides, wherein the overlapping peptides comprise all or part of the amino acid sequence of one or more stimulatory antigens;
selecting or enriching from the culture a plurality of lymphocytes (e.g., T cells) selectively stimulated by the one or more stimulatory antigens;
expanding the plurality of selectively stimulated lymphocytes (e.g., T cells); and
formulating the expanded, selectively stimulated lymphocytes (e.g., T cells) as a pharmaceutical composition.

13. A method of conferring an immune response to a subject having a tumor or a cancer, the method comprising:
obtaining a sample of PBMCs from the subject;
isolating from the sample of PBMCs a population of lymphocytes (e.g., T cells) and a population of monocytes;
differentiating the monocytes into dendritic cells;
selecting one or more stimulatory antigens by:
a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject;
b) contacting the bacterial cells or beads with antigen presenting cells (APCs) (e.g., a first batch of the dendritic cells), wherein the APCs internalize the bacterial cells or beads;
c) contacting the APCs with a first batch of the population of lymphocytes, under conditions suitable for activation of lymphocytes by a polypeptide presented by one or more APCs;
d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer;
co-culturing a second batch of the dendritic cells with (i) a second batch of the population of lymphocytes (e.g., T cells), and (ii) a plurality of overlapping peptides, wherein the overlapping peptides comprise all or part of the amino acid sequence of one or more stimulatory antigens;
selecting or enriching from the culture a plurality of lymphocytes (e.g., T cells) selectively stimulated by the one or more stimulatory antigens;
expanding the plurality of selectively stimulated lymphocytes (e.g., T cells); and
administering the expanded, selectively stimulated lymphocytes (e.g., T cells) to the subject, thereby conferring an immune response to the tumor or cancer.

14. A method of treating a subject having a tumor or a cancer, the method comprising:
obtaining a sample of PBMCs from the subject;
isolating from the sample of PBMCs a population of lymphocytes (e.g., T cells) and a population of monocytes;
differentiating the monocytes into dendritic cells;
selecting one or more stimulatory antigens by:
a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject;
b) contacting the bacterial cells or beads with antigen presenting cells (APCs) (e.g., a first batch of the dendritic cells), wherein the APCs internalize the bacterial cells or beads;
c) contacting the APCs with a first batch of the population of lymphocytes, under conditions suitable for activation of lymphocytes by a polypeptide presented by one or more APCs;
d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer;
co-culturing a second batch of the dendritic cells with (i) a second batch of the population of lymphocytes (e.g., T cells), and (ii) a plurality of overlapping peptides, wherein the overlapping peptides comprise all or part of the amino acid sequence of one or more stimulatory antigens;
selecting or enriching from the culture a plurality of lymphocytes (e.g., T cells) selectively stimulated by the one or more stimulatory antigens;
expanding the plurality of selectively stimulated lymphocytes (e.g., T cells); and
administering the expanded, selectively stimulated lymphocytes (e.g., T cells) to the subject, thereby treating the tumor or cancer.

15. The method of any one of claims 1-14, wherein the library comprises bacterial cells or beads comprising at least 1, 3, 5, 10, 15, 20, 25, 30, 50, 100, 150, 250, 500, 750, 1000 or more different heterologous polypeptides, or portions thereof.

16. The method of any one of claims 1-15, wherein determining whether one or more lymphocytes are activated by, or not responsive to, one or more polypeptides comprises measuring a level of one or more immune mediators.

17. The method of any one of claims 1-16, wherein the one or more immune mediators are selected from the group consisting of cytokines, soluble mediators, and cell surface markers expressed by the lymphocytes.

18. The method of any one of claims 1-17, wherein the one or more immune mediators are cytokines.

19. The method of claim 18, wherein the one or more cytokines are selected from the group consisting of TRAIL, IFN-gamma, IL-12p70, IL-2, TNF-alpha, MIP1-alpha, MIP1-beta, CXCL9, CXCL10, MCP1, RANTES, IL-1 beta, IL-4, IL-6, IL-8, IL-9, IL-10, IL-13, IL-15, CXCL11, IL-3, IL-5, IL-17, IL-18, IL-21, IL-22, IL-23A, IL-24, IL-27, IL-31, IL-32, TGF-beta, CSF, GM-CSF, TRANCE (also known as RANK L), MIP3-alpha, and fractalkine.

20. The method of any one of claims 1-17, wherein the one or more immune mediators are soluble mediators.

21. The method of claim 20, wherein the one or more soluble mediators are selected from the group consisting of granzyme A, granzyme B, granzyme K, sFas, sFasL, perforin, and granulysin.

22. The method of any one of claims 1-17, wherein the one or more immune mediators are cell surface markers.

23. The method of claim 22, wherein the one or more cell surface markers are selected from the group consisting of CD107a, CD107b, CD25, CD69, CD45RA, CD45RO, CD137 (4-1BB), CD44, CD62L, CD27, CCR7, CD154 (CD40L), KLRG-1, CD71, HLA-DR, CD122 (IL-2RB), CD28, IL7Ra (CD127), CD38, CD26, CD134 (OX-40), CTLA-4 (CD152), LAG-3, TIM-3 (CD366), CD39, PD1 (CD279), FoxP3, TIGIT, CD160, BTLA, 2B4 (CD244), CCR2, CCR5, CX3CR1, NKG2D, CD39, KLRD1, LGALS1 (encoding Galectin-1), and KLRG1.

24. The method of any one of claims 1-23, wherein lymphocyte activation is determined by assessing a level of one or more expressed or secreted immune mediators that is at least 20%, 40%, 60%, 80%, 100%, 120%, 140%, 160%, 180%, or 200% higher or lower than a control level.

25. The method of any one of claims 1-23, wherein lymphocyte activation is determined by assessing a level of one or more expressed or secreted immune mediators that is at least one, two, or three standard deviations greater or lower than the mean of a control level.

26. The method of any one of claims 1-23, wherein lymphocyte activation is determined by assessing a level of one or more expressed or secreted immune mediators that is at least 1, 2, 3, 4 or 5 median absolute deviations (MADs) greater or lower than a median response level to a control.

27. The method of any one of claims 1-23, wherein lymphocyte non-responsiveness is determined by assessing a level of one or more expressed or secreted immune mediators that is within 5%, 10%, 15%, or 20% of a control level.

28. The method of any one of claims 1-23, wherein lymphocyte non-responsiveness is determined by assessing a level of one or more expressed or secreted immune mediators that is less than one or two standard deviation higher or lower than the mean of a control level.

29. The method of any one of claims 1-23, wherein lymphocyte non-responsiveness is determined by assessing a level of one or more expressed or secreted immune mediators that is less than one or two median absolute deviation (MAD) higher or lower than a median response level to a control.

30. The method of any one of claims 1-29, wherein the one or more stimulatory antigens comprise (i) a tumor antigen described herein (e.g., comprising an amino acid sequence described herein), (ii) a polypeptide having an amino acid sequence at least 90%, 91%, 92%, 93%, 94%, 95%, 96%, 97%, 98%, 99% identical to the amino acid sequence of a tumor antigen described herein, and/or (iii) a polypeptide comprising the amino acid sequence of a tumor antigen described herein having at least one mutation, deletion, insertion, and/or translocation.

31. The method of any one of claims 1-30, further comprising producing the plurality of overlapping peptides.

32. The method of any one of claims 8-31, further comprising administering the composition comprising the selectively stimulated lymphocytes to the subject.

33. The method of claim 32, wherein the composition is administered to the subject by intravenous infusion.

34. The method of claim 32, wherein the subject suffers from refractory disease.

35. The method of claim 34, wherein the subject suffers from advanced refractory disease.

36. The method of claim 32, wherein the subject suffers from a solid tumor.

37. The method of claim 32, wherein the subject suffers from melanoma, malignant melanoma (MM), Merkel cell carcinoma (MCC), cutaneous squamous cell carcinoma (CSCC), non-small cell lung cancer (NSCLC), small cell lung cancer (SCLC), large cell lung cancer (LCLC), tracheobronchial cancer, pleomorphic carcinoma, squamous cell lung carcinoma (SqCLC), squamous cell carcinoma of the head and neck (SCCHN), nasopharyngeal carcinoma (NPC), urothelial carcinoma (bladder, ureter, urethra, or renal pelvis), renal cell carcinoma (RCC), or anal squamous cell carcinoma (ASCC).

38. The method of claim 32, further comprising administering to the subject a cancer therapy or combination of cancer therapies, e.g., a therapeutic cancer vaccine, a chemotherapeutic agent, an immune stimulator, or an immune checkpoint therapy.

39. A method of manufacturing a pharmaceutical composition, the method comprising:
obtaining a sample of PBMCs from a subject having a tumor or a cancer;
isolating from the sample of PBMCs a population of lymphocytes (e.g., T cells) and a population of CD14+ monocytes;
separating the sample of lymphocytes into a first batch of lymphocytes and a second batch of lymphocytes;
separating the sample of monocytes into a first batch of monocytes and a second batch of monocytes;
cryopreserving the first and second batches of monocytes and the first and second batches of lymphocytes and storing each cryopreserved batch for a specified period of time;
thawing the first batch of lymphocytes and/or the first batch of monocytes;
differentiating the first batch of monocytes into a first batch of dendritic cells;
selecting one or more stimulatory antigens by:
a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject;
b) contacting the bacterial cells or beads with the first batch of dendritic cells, wherein the dendritic cells internalize the bacterial cells or beads;
c) contacting the first batch of dendritic cells with the first batch of lymphocytes, under conditions suitable for activation of lymphocytes by a polypeptide presented by one or more of the dendritic cells;
d) determining whether one or more lymphocytes are activated by, or not responsive to, one or more polypeptides presented by one or more of the dendritic cells, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer;
synthesizing a plurality of overlapping peptides, wherein the overlapping peptides comprise all or part of the amino acid sequence of one or more stimulatory antigens;
thawing the second batch of lymphocytes and the second batch of monocytes;
differentiating the second batch of monocytes into a second batch of dendritic cells;
co-culturing the second batch of dendritic cells with (i) the second batch of lymphocytes (e.g., T cells), and (ii) the plurality of overlapping peptides, thereby selectively stimulating the second batch of lymphocytes;
selecting or enriching from the co-culture a plurality of lymphocytes (e.g., T cells) selectively stimulated by the one or more stimulatory antigens and selectively expanding the lymphocytes in the presence of one or more cytokines;
restimulating the selectively expanded, selectively stimulated lymphocytes with the plurality of overlapping peptides and sorting the lymphocytes (e.g., T cells) that express CD137+, CD154+, or CD154+ and CD137+ cell surface markers;
further expanding the plurality of selectively stimulated lymphocytes (e.g., T cells) by culturing the sorted lymphocytes in the presence of one or more cytokines and anti-CD3, anti-CD28, and/or anti-CD2 antibodies;
formulating the further expanded selectively stimulated lymphocytes (e.g., T cells) as a pharmaceutical composition; and
cryopreserving the pharmaceutical composition.

40. A method of manufacturing a pharmaceutical composition, the method comprising:
obtaining a sample of PBMCs from a subject having a tumor or a cancer;
isolating from the sample of PBMCs a population of lymphocytes (e.g., T cells) and a population of CD14+ monocytes;
separating the sample of lymphocytes into a first batch of lymphocytes and a second batch of lymphocytes;
separating the sample of monocytes into a first batch of monocytes and a second batch of monocytes;
differentiating the first batch of monocytes into a first batch of dendritic cells;
selecting one or more stimulatory antigens by:
a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject;
b) contacting the bacterial cells or beads with the first batch of dendritic cells, wherein the dendritic cells internalize the bacterial cells or beads;
c) contacting the first batch of dendritic cells with the first batch of lymphocytes, under conditions suitable for activation of lymphocytes by a polypeptide presented by one or more of the dendritic cells;
d) determining whether one or more lymphocytes are activated by, or not responsive to, one or more polypeptides presented by one or more of the dendritic cells, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer;
synthesizing a plurality of overlapping peptides, wherein the overlapping peptides comprise all or part of the amino acid sequence of one or more stimulatory antigens;
differentiating the second batch of monocytes into a second batch of dendritic cells;
co-culturing the second batch of dendritic cells with (i) the second batch of lymphocytes (e.g., T cells), and (ii) the plurality of overlapping peptides, thereby selectively stimulating the second batch of lymphocytes;
selecting or enriching from the co-culture a plurality of lymphocytes (e.g., T cells) selectively stimulated by the one or more stimulatory antigens and selectively expanding the lymphocytes in the presence of one or more cytokines;
restimulating the selectively expanded, selectively stimulated lymphocytes with the plurality of overlapping peptides and sorting the lymphocytes (e.g., T cells) that express CD137+, CD154+, or CD137+ and CD154+ cell surface markers;
further expanding the plurality of selectively stimulated lymphocytes (e.g., T cells) by culturing the sorted lymphocytes in the presence of one or more cytokines and anti-CD3, anti-CD28, and/or anti-CD2 antibodies; and
formulating the further expanded, selectively stimulated lymphocytes (e.g., T cells) as a pharmaceutical composition.

41. The method of any one of claims 39-40, wherein the one or more cytokines comprises one or more cytokines are selected from the group consisting of: IL-2, IL-7, IL-15, and IL-21.

42. A pharmaceutical composition comprising a plurality of selectively stimulated lymphocytes, wherein the selectively stimulated lymphocytes are obtained by a method comprising:
obtaining a sample of PBMCs from a subject having a tumor or a cancer;
isolating from the sample of PBMCs a population of lymphocytes (e.g., T cells) and a population of monocytes;
differentiating the monocytes into dendritic cells;
selecting one or more stimulatory antigens by:
a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject;
b) contacting the bacterial cells or beads with antigen presenting cells (APCs) (e.g., a first batch of the dendritic cells) from the subject, wherein the APCs internalize the bacterial cells or beads;
c) contacting the APCs with a first batch of the population of lymphocytes from the subject, under conditions suitable for activation of lymphocytes by a polypeptide presented by one or more APCs;
d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer;
co-culturing a second batch of the dendritic cells with (i) a second batch of the population of lymphocytes (e.g., T cells), and (ii) a plurality of overlapping peptides, wherein the overlapping peptides comprise all or part of the amino acid sequence of the one or more stimulatory antigens; and
selecting or enriching from the culture a plurality of lymphocytes (e.g., T cells).

43. A pharmaceutical composition comprising expanded, selectively stimulated lymphocytes, wherein the expanded, selectively stimulated lymphocytes are obtained by a method comprising:
obtaining a sample of PBMCs from a subject having a tumor or a cancer;
isolating from the sample of PBMCs a population of lymphocytes (e.g., T cells) and a population of monocytes;
differentiating the monocytes into dendritic cells;
selecting one or more stimulatory antigens by:
a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject;
b) contacting the bacterial cells or beads with antigen presenting cells (APCs) (e.g., a first batch of the dendritic cells), wherein the APCs internalize the bacterial cells or beads;
c) contacting the APCs with a first batch of the population of lymphocytes, under conditions suitable for activation of lymphocytes by a polypeptide presented by one or more APCs;
(d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer;
co-culturing a second batch of the dendritic cells with (i) a second batch of the population of lymphocytes (e.g., T cells), and (ii) a plurality of overlapping peptides, wherein the overlapping peptides comprise all or part of the amino acid sequence of one or more stimulatory antigens;
selecting or enriching from the culture a plurality of lymphocytes (e.g., T cells) selectively stimulated by the one or more stimulatory antigens; and
expanding the plurality of selectively stimulated lymphocytes (e.g., T cells).

44. A pharmaceutical composition comprising expanded, selectively stimulated T cells, wherein the expanded, selectively stimulated T cells are obtained by a method comprising:
obtaining a sample of PBMCs from a subject having a tumor or a cancer;
isolating from the sample of PBMCs a population of lymphocytes (e.g., T cells) and a population of CD14+ monocytes;
separating the sample of lymphocytes into a first batch of lymphocytes and a second batch of lymphocytes;
separating the sample of monocytes into a first batch of monocytes and a second batch of monocytes;
cryopreserving the first and second batches of monocytes and the first and second batches of lymphocytes and storing each cryopreserved batch for a specified period of time;
thawing the first batch of lymphocytes and/or the first batch of monocytes;
differentiating the first batch of monocytes into a first batch of dendritic cells; selecting one or more stimulatory antigens by:
a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject;
b) contacting the bacterial cells or beads with the first batch of dendritic cells, wherein the dendritic cells internalize the bacterial cells or beads;
c) contacting the first batch of dendritic cells with the first batch of lymphocytes, under conditions suitable for activation of lymphocytes by a polypeptide presented by one or more of the dendritic cells;
d) determining whether one or more lymphocytes are activated by, or not responsive to, one or more polypeptides presented by one or more of the dendritic cells, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer;
synthesizing a plurality of overlapping peptides, wherein the overlapping peptides comprise all or part of the amino acid sequence of one or more stimulatory antigens;
thawing the second batch of lymphocytes and the second batch of monocytes;
differentiating the second batch of monocytes into a second batch of dendritic cells;
co-culturing the second batch of dendritic cells with (i) the second batch of lymphocytes (e.g., T cells), and (ii) the plurality of overlapping peptides, thereby selectively stimulating the second batch of lymphocytes;
selecting or enriching from the co-culture a plurality of lymphocytes (e.g., T cells) selectively stimulated by the one or more stimulatory antigens and selectively expanding the lymphocytes in the presence of one or more cytokines;
restimulating the selectively expanded, selectively stimulated lymphocytes with the plurality of overlapping peptides and sorting the lymphocytes (e.g., T cells) that express CD137+, CD154+, or CD137+ and CD154+ cell surface markers; and
further expanding the plurality of selectively stimulated lymphocytes (e.g., T cells) by culturing the sorted lymphocytes in the presence of one or more cytokines and anti-CD3, anti-CD28, and/or anti-CD2 antibodies.

45. A pharmaceutical composition comprising expanded, selectively stimulated T cells, wherein the expanded, selectively stimulated T cells are obtained by a method comprising:
obtaining a sample of PBMCs from a subject having a tumor or a cancer;
isolating from the sample of PBMCs a population of lymphocytes (e.g., T cells) and a population of CD14+ monocytes;
separating the sample of lymphocytes into a first batch of lymphocytes and a second batch of lymphocytes;
separating the sample of monocytes into a first batch of monocytes and a second batch of monocytes;
differentiating the first batch of monocytes into a first batch of dendritic cells;
selecting one or more stimulatory antigens by:
a) obtaining, providing, or generating a library comprising bacterial cells or beads, wherein each bacterial cell or bead of the library comprises a different heterologous polypeptide comprising one or more mutations, viral sequences, splice variants, gene fusions, peptide fusions, or translocations expressed in a cancer or tumor cell of the subject;
b) contacting the bacterial cells or beads with the first batch of dendritic cells, wherein the dendritic cells internalize the bacterial cells or beads;
c) contacting the first batch of dendritic cells with the first batch of lymphocytes, under conditions suitable for activation of lymphocytes by a polypeptide presented by one or more of the dendritic cells;
d) determining whether one or more lymphocytes are activated by, or not responsive to, one or more polypeptides presented by one or more of the dendritic cells, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer;
synthesizing a plurality of overlapping peptides, wherein the overlapping peptides comprise all or part of the amino acid sequence of one or more stimulatory antigens;
differentiating the second batch of monocytes into a second batch of dendritic cells;
co-culturing the second batch of dendritic cells with (i) the second batch of lymphocytes (e.g., T cells), and (ii) the plurality of overlapping peptides, thereby selectively stimulating the second batch of lymphocytes;
selecting or enriching from the co-culture a plurality of lymphocytes (e.g., T cells) selectively stimulated by the one or more stimulatory antigens and selectively expanding the lymphocytes in the presence of one or more cytokines;
restimulating the selectively expanded, selectively stimulated lymphocytes with the plurality of overlapping peptides and sorting the lymphocytes (e.g., T cells) that express CD137+, CD154+, or CD137+ and CD154+ cell surface markers; and
further expanding the plurality of selectively stimulated lymphocytes (e.g., T cells) by culturing the sorted lymphocytes in the presence of one or more cytokines and anti-CD3, anti-CD28, and/or anti-CD2 antibodies.
